(19) United States
(12) Patent Application Publication
(10) Pub. No.: US 2026/0327243 A1
(43) Pub. Date: Oct. 22, 2026

(54) SPIRO-HETEROCYCLIC INHIBITORS OF KRAS G12C MUTANT PROTEINS AND USES THEREOF

(71) Applicant: AMGEN INC., THOUSAND OAKS, CA (US)

(72) Inventors: BRIAN ALAN LANMAN, WOODLAND HILLS, CA (US); ABHISEK BANERJEE, BANGALORE, KARNATAKA (IN); JOHN R. BUTLER, THOUSAND OAKS, CA (US); EMIL GLIBSTRUP, COPENHAGEN (DK); DAVID HUANG, THOUSAND OAKS, CA (US); BIRGITTE W. HUSEMOEN, VALBY (DK); MATTHEW R. KALLER, VENTURA, CA (US); TODD J. KOHN, THOUSAND OAKS, CA (US); SEBASTIAN LETH-PETERSEN, COPENHAGEN (DK); JOSE M. MEDINA, THOUSAND OAKS, CA (US); ALEXANDER J. PICKRELL, WESTLAKE VILLAGE, CA (US); SLAVKO RAST, VALBY (DK); NURIA A. TAMAYO, NEWBURY PARK, CA (US); HUI-LING WANG, THOUSAND OAKS, CA (US); WENHAN ZHANG, THOUSAND OAKS, CA (US)

(73) Assignee: AMGEN INC., THOUSAND OAKS, CA (US)

(21) Appl. No.: 19/480,913

(22) PCT Filed: May 3, 2024
(86) PCT No.: PCT/US2024/027587

§ 371 Date: Nov. 3, 2025

Related U.S. Application Data
(60) Provisional application No. 63/464,102, filed on May 4, 2023.
(60) Provisional application No. 63/632,098, filed on Apr. 10, 2024.

(51) Int. Cl. C07D 513/20; A61K 31/496; A61K 31/506; A61K 31/5386; A61K 45/06; A61P 35/00; C07D 221/20; C07D 401/14; C07D 471/10; C07D 491/107; C07D 491/113; C07D 491/20; C07D 491/22; C07D 495/20; C07D 498/10; C07D 498/20

(52) U.S. Cl. CPC C07D 513/20; A61K 31/496; A61K 31/506; A61K 31/5386; A61K 45/06; A61P 35/00; C07D 221/20; C07D 401/14; C07D 471/10; C07D 491/107; C07D 491/113; C07D 491/20; C07D 491/22; C07D 495/20; C07D 498/10; C07D 498/20

(57) ABSTRACT

The present disclosure provides compounds having activity as inhibitors of the G12C mutant KRAS protein, pharmaceutical compositions comprising the compounds, and methods of treating certain disorders, such as cancer, including but not limited to lung cancer, pancreatic cancer, colorectal cancer, and solid tumors. In particular, the disclosure provides compounds of Formula (II):, and pharmaceutically acceptable salts thereof, wherein the substituents are as described.

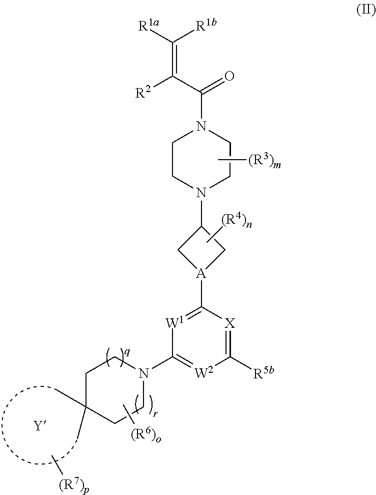

### CROSS-REFERENCE TO RELATED APPLICATIONS

This application claims the benefit of priority to U.S. Provisional Patent Application Nos. 63/464,102, filed on May 4, 2023, and 63/632,098, filed Apr. 10, 2024, each of which is hereby incorporated by reference in its entirety for all purposes, as if fully set forth herein.

### FIELD

The present disclosure relates generally to compounds having activity as inhibitors of the G12C-mutant KRAS protein, pharmaceutical compositions comprising the compounds, and uses and methods of treating disorders such as cancer, including but not limited to lung, pancreatic and colorectal cancer.

### BACKGROUND

The KRAS oncoprotein is a G-protein that couples extracellular mitogenic signaling to intracellular, pro-proliferative responses, KRAS functions as a molecular “on/off” switch, alternating between an inactive GDP-bound state and an active GTP-bound state. Transition between these states is facilitated by guanine nucleotide-exchange factors. Mitogen stimulation can induce GTP binding, which results in a conformational change that enables KRAS to interact with downstream effector proteins, leading to cellular proliferation. In normal cells, the pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation. See Simanshu et al., Cell 2017, 170, 17-33.

Attempts to develop inhibitors of mutated KRAS proteins have historically been thwarted by the picomolar affinity with which KRAS binds to GDP and GTP, as well as the absence of druggable pockets on the surface of the protein. See Cox et al., Nat. Rev. Drug Discov. 2014, 13, 828-851. Covalent inhibitors of the G12C mutant of KRAS (“KRAS^{G12C}”) have been identified. These inhibitors can bind to a previously unrecognized allosteric pocket on GDP-KRAS^{G12C}, preventing its subsequent activation. See O'Bryan, J. P. Pharmacol. Res. 2019, 139, 503-511 and Ostrem et al., Nature 2013, 503, 548-551. This discovery brought about significant new efforts in KRAS inhibitor research, recently culminating in the entry of KRAS inhibitors into human clinical trials.

### SUMMARY

One aspect of the disclosure provides compounds of Formula (II):

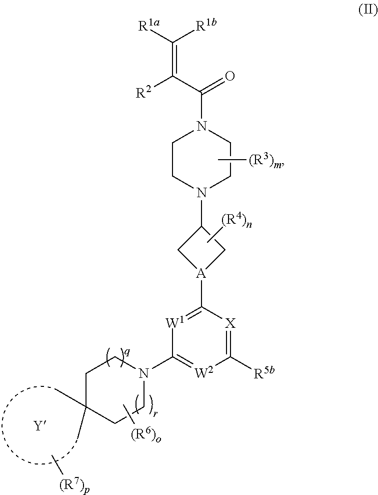

or pharmaceutically acceptable salts thereof,
wherein:
m is 0, 1, 2, 3, or 4;
n is 0, 1, or 2;
is 0, 1, 2, 3, or 4;
p is 0, 1, 2, 3, 4, or 5;
q is 0, 1, or 2;
r is 0 or 1;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃haloalkyl, C—C₀₋₃alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkyleneOH, or C₀₋₃alkyleneC₁₋₄alkoxy;
X is N or C—R^{5a};
Y′ is C₃₋₇cycloalkyl, C₄₋₇cycloalkenyl, heterocycloalkyl having 3 to 7 total ring atoms and 1-3 heteroatoms independently selected from N, O, and S, or heterocycloalkenyl having 5 to 7 total ring atoms and 1-3 heteroatoms independently selected from N, O, and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form

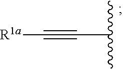

each R³ independently is C₁₋₃alkyl, C₁₋₃haloalkyl,

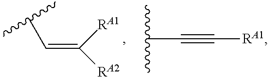

C₀₋₃alkyleneCN, C₀₋₃alkyleneOH, C₀₋₃alkylene-C₁₋₃alkoxy, oxo, spiro-C₃₋₇cycloalkyl, spiro-C₄₋₇cycloalkenyl, spiro-heterocycloalkyl having 3-7 total ring atoms and 1 or 2 heteroatoms independently selected from N, O, and S, or spiro-heterocycloalkenyl having 4-7 total ring atoms and 1 or 2 heteroatoms independently selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms independently selected from N, O, and S, or fused-heterocycloalkenyl having 4-7 total ring atoms and 1 or 2 heteroatoms independently selected from N, O, and S;
each of R^{A1} and R^{A2} independently is H, C₁₋₃alkyl, C₁₋₃haloalkyl, or C₃₋₅cycloalkyl;
each R⁴ independently is C₁₋₃alkyl, C₁₋₃haloalkyl, C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 beteroatoms independently selected from N, O and S;
R^{5a} is H, CN, halo, C₁₋₃alkyl, C₁₋₃haloalkyl, C₂₋₆alkenyl, C₂₋₆alkynyl, C₀₋₃alkyleneOH, C₀₋₃alkylene-C₁₋₃alkoxy, or C₃₋₅cycloalkyl;
R^{5b} is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms independently selected from N, O, and S, heterocycloalkenyl having 5-7 total ring atoms and 1-3 heteroatoms independently selected from N, O, and S, or R^{5a} and R^{5b}, together with the atoms to which they are attached, form C₃₋₇cycloalkyl;
wherein each of the foregoing C₁₋₆alkyl, C₂₋₄alkenyl, C₂₋₄alkynyl, C₃₋₇cycloalkyl, C₅₋₇cycloalkenyl, heterocycloalkyl, and heterocycloalkenyl independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl;
each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇-cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal Re, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

Yet another aspect of the disclosure provides a compound listed in Table A, Table B, Table C, or Table D, or a pharmaceutically acceptable salt of any of the foregoing.

Another aspect of the disclosure provides a pharmaceutical composition comprising a compound disclosed herein (such as a compound of Formula (I), Formula (IA), Formula (IB), Formula (II), Formula (IIA), Formula (IIB), Formula (IIC), a compound listed in Table A, Table B, Table C, or Table D, or a pharmaceutically acceptable salt of any of the foregoing), and a pharmaceutically acceptable excipient.

Yet another aspect of the disclosure provides a method of treating cancer in a subject in need of treatment, the method comprising administering to the subject a therapeutically effective amount of a compound described herein (such as a compound of Formula (I), Formula (IA), Formula (IB), Formula (II), Formula (IIA), Formula (IIB), Formula (IIC), a compound listed in Table A, Table B, Table C, or Table D, or a pharmaceutically acceptable salt of any of the foregoing), or a pharmaceutical composition described herein.

Still another aspect of the disclosure provides a compound described herein (such as a compound of Formula (I), Formula (IA), Formula (IB), Formula (II), Formula (IIA), Formula (IIB), Formula (IIC), a compound listed in Table A, Table B, Table C, or Table D, or a pharmaceutically acceptable salt of any of the foregoing), or a pharmaceutical composition described herein, for use as a medicament.

Another aspect of the disclosure provides a compound disclosed herein (such as a compound of Formula (I), Formula (IA), Formula (IB), Formula (II), Formula (IIA), Formula (IIB), Formula (IIC), a compound listed in Table A, Table B, Table C, or Table D. or a pharmaceutically acceptable salt of any of the foregoing), or the pharmaceutical composition disclosed herein, for use in the treatment of cancer. Yet another aspect of the disclosure provides a compound disclosed herein (such as a compound of Formula (I), Formula (IA), Formula (IB), Formula (II), Formula (IIA), Formula (IIB), Formula (IIC), a compound listed in Table A, Table B, Table C, or Table D, or a pharmaceutically acceptable salt of any of the foregoing), or the pharmaceutical composition disclosed herein, for the manufacture of a medicament for the treatment of cancer. In some cases, the cancer of any of the foregoing is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Still another aspect of the disclosure provides a compound of Formula (B):

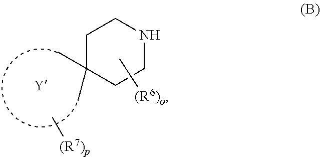

or a nitrogen-protected analog, or a pharmaceutically acceptable salt of any of the foregoing, wherein:
o is 0, 1, 2, 3, or 4;
p is 0, 1, 2, 3, 4, or 5;
each R⁶ s halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
   each R^{N1} independently is H or C₁₋₄alkyl.

Yet another aspect of the disclosure provides a method of preparing the compound or salt of the disclosure (such as a compound of Formula (I), Formula (IA), Formula (IB), Formula (II), Formula (IIA), Formula (IIB), Formula (IIC), a compound listed in Table A, Table B, Table C, or Table D, or a pharmaceutically acceptable salt of any of the foregoing), comprising admixing a compound of Formula (B):

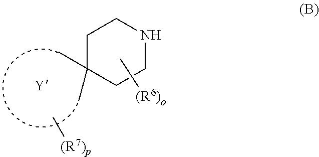

with a compound:

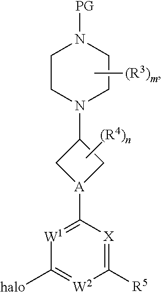

under carbon-nitrogen bond-forming conditions to form:

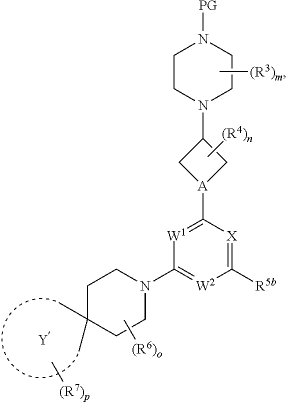

wherein PG is a protecting group

Still another aspect of the disclosure provides a method of preparing the compound or salt of the disclosure (such as a compound of Formula (I), Formula (IA), Formula (IB), Formula (II), Formula (IIA), Formula (IIB), Formula (IIC), a compound listed in Table A, Table B, Table C, or Table D. or a pharmaceutically acceptable salt of any of the foregoing), comprising converting a compound listed in Table E, Table F, Table G, a compound selected from selected from the group consisting of 3-001.1, 3-001.2, 3-002.1, 3-002.2, 3-003.1, 3-003.2, 3-020.1, 3-020.2, 3-021.1, 3-021.2, 3-046.1, 3.046.2, 3-050.1, 3-050.2, 3-051.1, 3-051.2, 3-051.3, 3-051.4, 3-051.5, 3-052.1, 3-052.2, 3-052.3, 3-052.4, 3-052.5, 3-053.1, 3-053.2, 3-053.3, 3-053.4, 3-053.5, 3-054-1, 3-054-2, 3-055.1, 3-055.2, 3-055.3, 3-055.4, 3-055.5, 3-057.1, 3-057.2, 3-057.3, 3-058.1, 3-058.2-1, 3-058.2.2, 3-058.3, 3-059.1, 3-060.1, 3-060.2, 3-060.3, 3-060.4, 3-060.5, 3-060.6, 3-060.7, 3-060.8, 3-060.9, 3-061.1, 3-061.2, 3-061.3, 3-061.4, 3-061.5, 3-062.1-1, 3-062.1-2, 3-062.1-1, 3-062.2, 3-063.1, 3-063.2, 3-065.1, 3-065.2, 3-065.3, 3-065.4, 3-065.5, 3-067-1, 3-067-2, 3-069.1, 3-069.2, 3-073.1, 3-073.2, 3-073.3, 3-073.4, 3-073.5, 3-074.1, 3-074.2, 3-074.3, 3-074.4, 3-075.1, 3-075.2, 3-075.3, 3-076.1, and 3-076.2, a protected analog of any of the foregoing, or a pharmaceutically acceptable salt of any of the foregoing, into a compound of the disclosure.

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein.

### DETAILED DESCRIPTION

Disclosed herein are compounds having activity as inhibitors of the G12C-mutant KRAS protein, pharmaceutical compositions comprising the compounds, and uses and methods of treating disorders, such as cancer, with the compounds and pharmaceutical composition described herein.

### Compounds of Formula (II)

Provided herein are compounds of Formula (II):

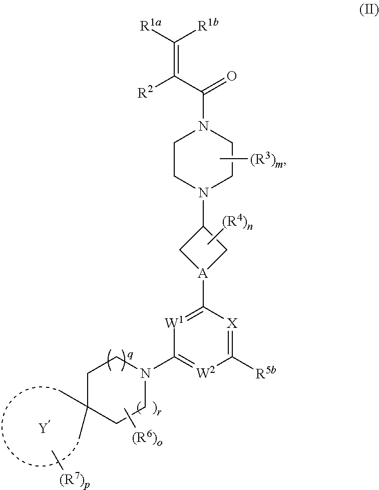

or pharmaceutically acceptable salts thereof,
wherein:
m is 0, 1, 2, 3, or 4;
n is 0, 1, or 2;
o is 0, 1, 2, 3, or 4;
p is 0, 1, 2, 3, 4, or 5;
q is 0, 1, or 2;
r is 0 or 1;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃ haloalkyl, C—C₀₋₃alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkylencOH, or C₀₋₃alkyleneC₁₋₄alkoxy;
X is N or C—R^{5a},
Y′ is C₃₋₇cycloalkyl, C₄₋₇cycloalkenyl, heterocycloalkyl having 3 to 7 total ring atoms and 1-3 heteroatoms independently selected from N, O, and S, or heterocycloalkenyl having 5 to 7 total ring atoms and 1-3 heteroatoms independently selected from N, O, and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form

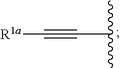

each R³ independently is C₁₋₃alkyl, C₁₋₃haloalkyl,

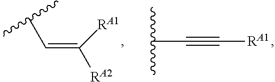

C₀₋₃alkyleneCN, C₀₋₃alkyleneOH, C₀₋₃alkylene-C₁₋₃alkoxy, oxo, spiro-C₃₋₇cycloalkyl, spiro-C₄₋₇cycloalkenyl, spiro-heterocycloalkyl having 3-7 total ring atoms and 1 or 2 heteroatoms independently selected from N, O, and S, or spiro-heterocycloalkenyl having 4-7 total ring atoms and 1 or 2 heteroatoms independently selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms independently selected from N, O, and S, or fused-heterocycloalkenyl having 4-7 total ring atoms and 1 or 2 heteroatoms independently selected from N, O, and S;
each of R^{A1} and R^{A2} independently is H, C₁₋₃alkyl, C₁₋₃haloalkyl, or C₃₋₅cycloalkyl;
each R⁴ independently is C₁₋₃alkyl, C₁₋₃haloalkyl, C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 heteroatoms independently selected from N, O and S;
R^{5a} is H, CN, halo, C₁₋₃alkyl, C₁₋₃haloalkyl, C₂₋₆alkenyl, C₂₋₆alkynyl, C₀₋₃alkyleneOH, C₀₋₃alkylene-C₁₋₃alkoxy, or C₃₋₅cycloalkyl;
R^{5b} is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms independently selected from N, O, and S, heterocycloalkenyl having 5-7 total ring atoms and 1-3 heteroatoms independently selected from N, O, and S, or R^{5a} and R^{5b}, together with the atoms to which they are attached, form C₃₋₇cycloalkyl;
wherein each of the foregoing C₁₋₆alkyl, C₂₋₆alkenyl, C₂₋₄alkynyl, C₃₋₇cycloalkyl, C₅₋₇cycloalkenyl, heterocycloalkyl, and heterocycloalkenyl independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl;
each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N. O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring Rejoin together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

In some cases, the compound of Formula (II) is a free base. In some cases, the compound of Formula (II) is a pharmaceutically acceptable salt.

In some cases, R^{1a} is H or D. In some cases, R^{1a} is H. In some cases. R^{1a} is D. In some cases, R^{1b} is H or D. In some cases, R^{1b} is H. In some cases, R^{1b} is D. In some cases, R² is H or D. In some cases, R² is H. In some cases, R² is D. In some cases, at least one of R^{1a}, R^{1b}, and R² is H or D. In some cases, at least one of R^{1a}, R^{1b}, and R² is H. In some cases, at least one of R^{1a}, R^{1b}, and R² is D. In some cases, at least two of R^{1a}, R^{1b}, and R² are each independently H or D. In some cases, at least two of R^{1a}, R^{1b}, and R² are H. In some cases, at least two of R^{1a}, R^{1b}, and R² are D. In some cases, each of R^{1a}, R^{1b}, and R² independently is H or D. In some cases, two of R^{1a}, R^{1b}, and R² are H and one of R^{1a}, R^{1b}, and R² is halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, or C₁₋₂alkylene-heterocycloalkyl having 3-6 total ring atoms and 1 or 2 heteroatoms independently selected from N, O, and S. In some cases, each of R^{1a}, R^{1b}, and R² is H. In some cases, each of R^{1a}, R^{1b}, and R² is D. In some cases, at least one of R^{1a}, R^{1b}, and R² is halo. In some cases, one of R^{1a}, R^{1b}, and R² is halo. In some cases. R^{1a} is halo and each of R^{1b} and R² is H. In some cases, at least one of R^{1a}, R^{1b}, and R² is Br, Cl, or F. In some cases, one of R^{1a}, R^{1b}, and R² is Br, Cl, or F. In some cases, R^{1a} is Br, Cl, or F and each of R^{1b} and R² is H. In some cases, at least one of R^{1a}, R^{1b}, and R² is Br or Cl. In some cases, one of R^{1a}, R^{1b}, and R² is Br or Cl. In some cases. R^{1a} is Br or Cl and each of R^{1b} and R² is H. In some cases, at least one of R^{1a}, R^{1b}, and R² is C₁₋₄alkyl or C₁₋₄haloalkyl. In some cases, one of R^{1a}, R^{1b}, and R² is C₁₋₄alkyl or C₁₋₄haloalkyl. In some cases, at least one of R^{1a}, R^{1b}, and R² is CH₃, CH₂CH₃, CH₂CH₂CH₃, CH(CH₃)₂, CH₂CH₂CH₂CH₃, CH₂F, CHF₂, or CF₃. In some cases, one of R^{1a}, R^{1b}, and R² is CH₃, CH₂CH₃, CH₂CH₂CH₃, CH(CH₃)₂, CH₂CH₂CH₂CH₃, CH₂F, CHF₂, or CF₃. In some cases, at least one of R^{1a}, R^{1b}, and R² is CH₃, CH₂F, CHF₂, or CF₃. In some cases, one of R^{1a}, R^{1b}, and R² is CH₃, CH₂F, CHF₂, or CF₃. In some cases, at least one of R^{1a}, R^{1b}, and R² is C₁₋₂alkylene-OH, C₀₋₂alkylene-C₁₋₄alkoxy, C₀₋₂alkylene-C₁₋₄haloalkoxy, C₀₋₂alkylene-CN, or C₀₋₂alkylene-N(R^{N1}), and each R^{N1} independently is H or C₁₋₄alkyl. In some cases, each R^{N1} independently is H or CH₃. In some cases, each R^{N1} independently is H. In some cases, at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, at least one of R^{1a}, R^{1b}, and R² is C₁₋₂alkylene-heterocycloalkyl wherein theterocycloalkyl contains 3-6 total ring atoms and 1 or 2 heteroatoms independently selected from N, O, and S. In some cases, theterocycloalkyl is aziridinyl, oxiranyl, azetidinyl, oxetanyl, pyrrolidinyl, tetrahydrofuranyl, imidazolidinyl, pyrazolidinyl, oxathiolidinyl, isoxthiodinyl, oxazolidinyl, isoxazolidinyl, thiazolidinyl, isothiazolidinyl, piperidinyl, diazinyl, or morpholinyl. In some cases, theterocycloalkyl is aziridinyl, azetidinyl, pyrrolidinyl, piperidinyl, or morpholinyl. In some cases, at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₂, CH₂N(CH₃)₂, aziridin-1-yl-methyl, azetidin-1-yl-methyl, pyrrolidine-1-yl-methyl, piperidin-1-yl-methyl, or morpholin-1-yl-methyl. In some cases, R^{1b} and R², together with the carbon atoms to which they are attached, form

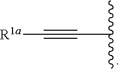

In some cases, R^{1a} is H. In some cases, R^{1b} and R² together with the carbon atoms to which they are attached form

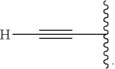 [SMILES: *C#C]

In some cases,

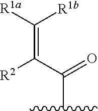

is

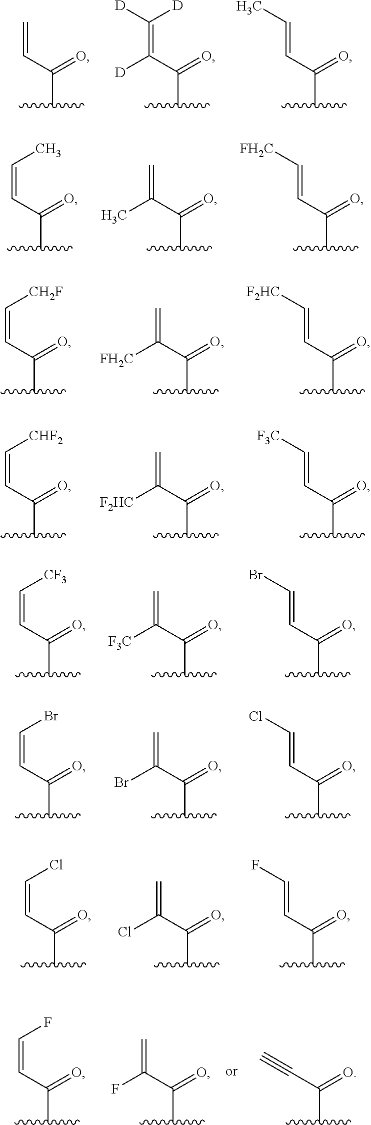

In some cases

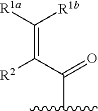

is

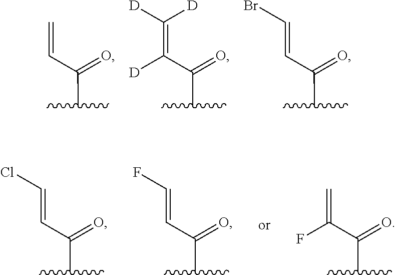

In some cases,

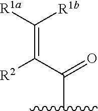

is

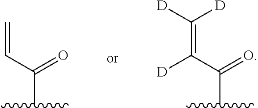

In some cases,

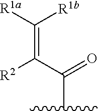

is

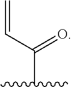 [SMILES: *C(=O)C=C]

In some cases, m is 0, and

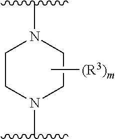

is

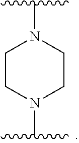 [SMILES: *N1CCN(*)CC1]

In some cases, m is 1. In some cases, m is 2. In some cases, m is 3. In some cases, m is 4. In some cases,

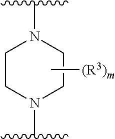

is deuterated. In some cases,

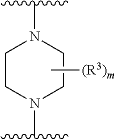

is fully deuterated and has a structure:

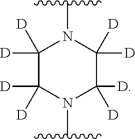

In some cases, at least one R³ is C₁₋₃alkyl or C₁₋₃haloalkyl. In some cases, at least one R³ is CH₃, CH₂CH₃, CH₂CH₂CH₃, CH(CH₃)₂, CF₃, CHF₂, or CH₂F. In some cases, at least one R³ is CH₃, CH₂CH₃, CF₃, CHF₂, or CH₂F. In some cases, at least one R³ is CH₃. In some cases, m is 1 or 2 and each R³ is CH₃. In some cases, m is 1 and R³ is CF₃, CHF₂, or CH₂F. In some cases, at least one R³ is

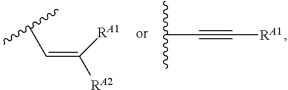

and each of R^{A1} and R^{A2} independently is H, C₁₋₃alkyl, C₁₋₃haloalkyl, or C₃₋₅cycloalkyl. In some cases, m is 1 and R³ is

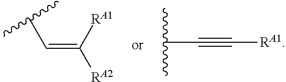

In some cases, each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl. In some cases,

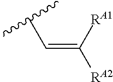

is

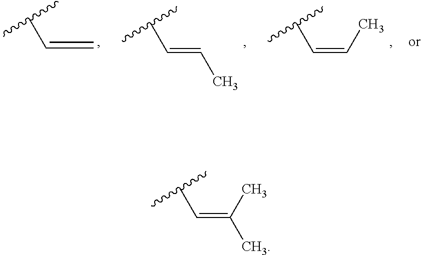

In some cases,

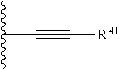

is

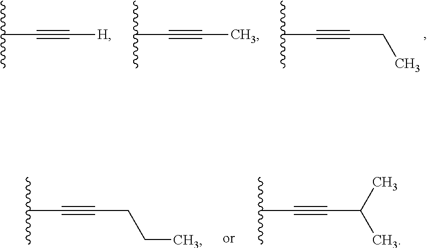

In some cases,

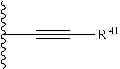

is

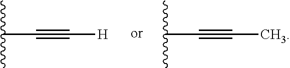

In some cases, at least one R³ is

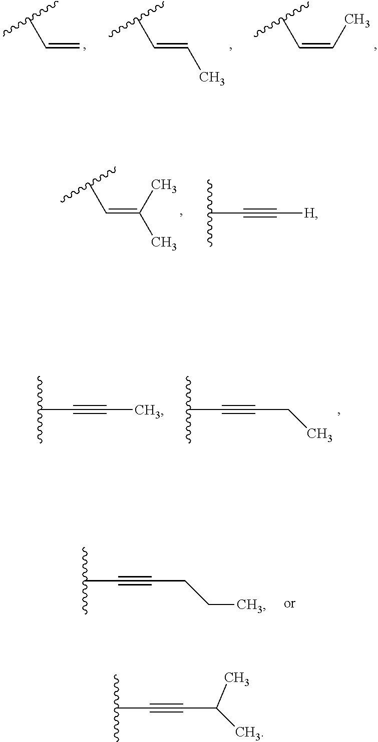

In some cases, at least one R³ is

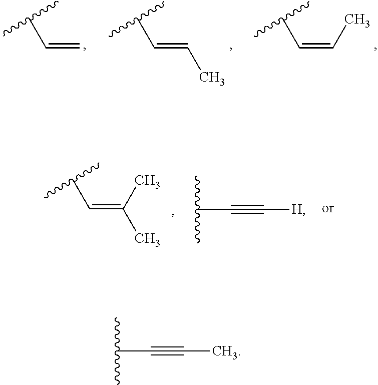

In some cases, at least one R³ is C₀₋₃alkylencCN. In some cases, at least one R³ is CN, CH₂CN, or CH₂CH₂CN. In some cases, at least one R³ is CN or CH₂CN. In some cases, m is 1 and R³ is CN or CH₂CN. In some cases, at least one R³ is C₀₋₃alkyleneOH or C₀₋₃alkylene-C₁₋₃alkoxy. In some cases, at least one R³ is OH, CH₂OH, CH₂CH₂OH, OCH₃, CH₂OCH₃, or CH₂CH₂OCH₃. In some cases, m is 1 and R³ is OH, CH₂OH, CH₂CH₂OH, OCH₃, CH₂OCH₃, or CH₂CH₂OCH₃. In some cases, at least one R³ is oxo (═O). In some cases, at least one R³ is spiro-C₃₋₇cycloalkyl or spiro-heterocycloalkyl having 3-7 total ring atoms and 1 or 2 heteroatoms independently selected from N, O and S. In some cases, at least one R³ is spiro-cyclopropyl, spiro-cyclobutyl, or spiro-cyclopentyl. In some cases, at least one R³ is spiro-azetidinyl, spiro-oxetanyl, spiro-pyrrolidinyl, spiro-imidazolidinyl, spiro-pyrazolidinyl, or spiro-tetrahydrofuranyl. In some cases, at least one R³ is spiro-cyclopropyl, spiro-cyclobutyl, spiro-cyclopentyl, spiro-azetidinyl, spiro-oxetanyl, spiro-pyrrolidinyl, spiro-imidazolidinyl, spiro-pyrazolidinyl, or spiro-tetrahydrofuranyl. In some cases, at least one R³ is spiro-cyclopropyl, spiro-cyclobutyl, spiro-oxetanyl, or spiro-tetrahydrofuranyl. In some cases, at least one R³ is spiro-C₄₋₇cycloalkenyl or spiro-heterocycloalkenyl having 4-7 total ring atoms and 1 or 2 heteroatoms independently selected from N, O and S. In some cases, two vicinal R³, together with the atoms to which they are attached, form fused-C₃₋₇cycloalkyl or fused-heterocycloalkyl having 3-7 total ring atoms and 1 or 2 heteroatoms independently selected from N, O and S. In some cases, two vicinal R³, together with the atoms to which they are attached, form fused-cyclopropyl, fused-cyclobutyl, fused-cyclopentyl, or fused-cyclohexyl. In some cases, two vicinal R³, together with the atoms to which they are attached, form fused-cyclopropyl or fused-cyclobutyl. In some cases, two vicinal R³, together with the atoms to which they are attached, form fused-C₄₋₇cycloalkenyl or fused-heterocycloalkenyl having 4-7 total ring atoms and 1 or 2 heteroatoms independently selected from N, O and S. In some cases, each R³ independently is CH₃, CH₂CH₃, CF₃, CHF₂, CH₂F,

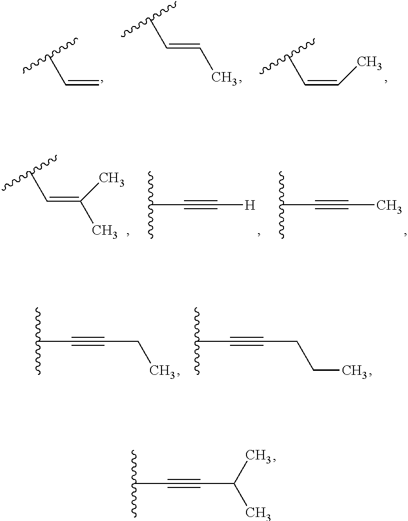

CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl. In some cases, each R³ independently is CH₃, CH₂CH₃, CH₂F, CHF₂, CF₃, CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl. In some cases, m is 0; or m is 1 and R³ is CH₃, CH₂F, CHF₂, CF₃, CN, CH₂CN, CH₂OH, CH₂OCH₃, or spiro-oxetanyl. In some cases, m is 0; or m is 1 and R³ is CH₃. In some cases,

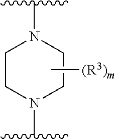

is

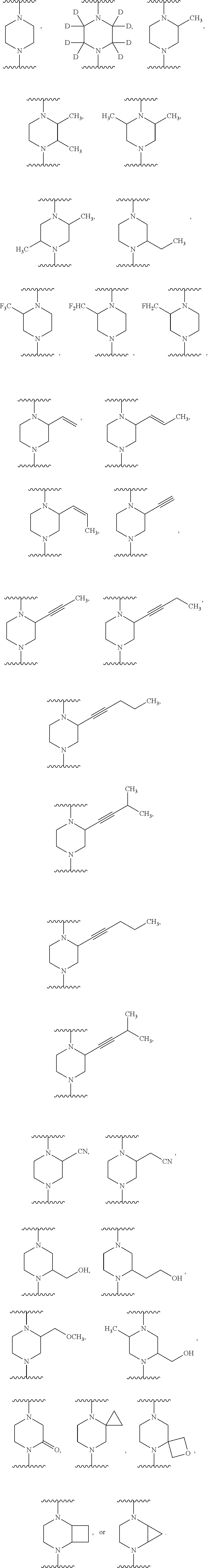

In some cases,

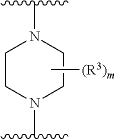

is

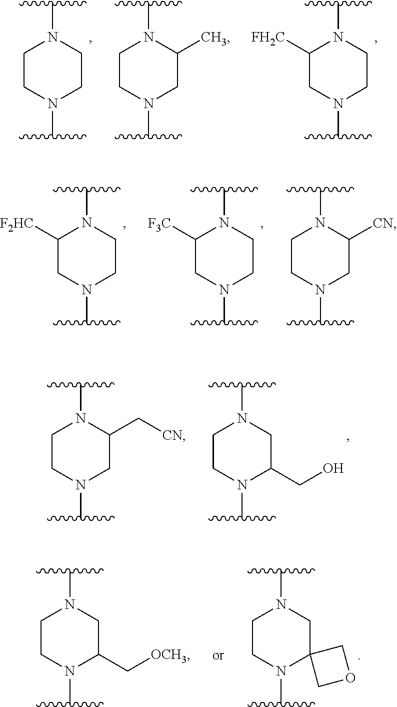

In some cases,

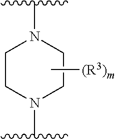

is

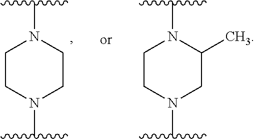 [SMILES: *N1CCN(*)CC1 ; *N1CCN(*)C(C)C1]

In some cases,

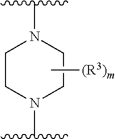

is

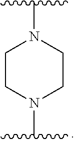 [SMILES: *N1CCN(*)CC1]

In some cases,

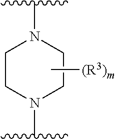

is

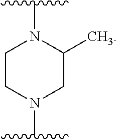 [SMILES: *N1CCN(*)C(C)C1]

In some cases, A is N. In some cases, A is CH, C-halo, C—CN, C—C₁₋₃alkyl, C—C₁₋₃haloalkyl, C—C₀₋₃alkyleneOH, or C—C₀₋₃alkylene-C₁₋₄alkoxy. In some cases, A is CH. In some cases, A is C—F, C—Cl, or C—CN. In some cases, A is C-halo or C—CN. In some cases. A is C—F or C—Cl. In some cases, A is C—F. In some cases, A is C—CN. In some cases, A is C—C₁₋₃alkyl. In some cases, A is C—CH₃, C—CH₂CH₃, C—CH—CH₂CH₃, or C—CH(CH₃)₂. In some cases, A is C—CH₃. In some cases, A is C—C₁₋₃haloalkyl. In some cases, A is C—CF₃, C—CHF₂, or C—CH₂F. In some cases, A is C—CH₃, C—CH₂CH₃, C—CH₂CH₂CH₃, C—CH(CH₃)₂, C—CF₃, C—CHF₂, or C—CH₂F. In some cases, A is C—CH₃, C—CH₂F, C—CHF₂, or C—CF₃. In some cases, A is C—C₀₋₃alkyleneOH or C—C₀₋₃alkylene-C₁₋₄alkoxy. In some cases, A is C—OH, C—CH₂OH, C—CH₂CH₂OH, C—OCH₃, C—CH₂OCH₃, or C—CH₂CH₂OCH₃. In some cases, A is C—OH, C—CH₂OH, C—OCH₃, or C—CH₂OCH₃. In some cases, A is CH, C—F, C—Cl, C—CN, C—CH₃, C—CH₂F, C—CHF₂, C—CF₃, C—OH, C—CH₂OH, C—OCH₃, or C—CH₂OCH₃. In some cases, A is N, CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, A is N, CH, C—F, C—Cl, C—CN, C—CH₃, C—CF₃, C—CHF₂, C—CH₂F, C—OH, C—CH₂OH, C—OCH₃, or C—CH₂OCH₃. In some cases, A is N, CH, or C—CH₃. In some cases, n is 0. In some cases, n is 1. In some cases, n is 2. In some cases, at least one R⁴ is C₁₋₃alkyl or C₁₋₃haloalkyl. In some cases, at least one R⁴ is CH₃, CH₂CH₃, CH₂CH₂CH₃, CH(CH₃)₂, CF₃, CHF₂, or CH₂F. In some cases, at least one R⁴ is CH₃. In some cases, one R⁴ is CH₃, CH₂CH₃, CHCH₂CH₃, CH(CH₃)₂, CF₃, CHF₂, or CH₂F. In some cases, n is 2 and each R⁴ independently is CH₃, CH₂CH₃, CH₂CH₂CH₃, CH(CH₃)₂, CF₃, CHF₂, or CH₂F. In some cases, n is 1 and R⁴ is CH₃, CH₂CH₃, CH₂CH₂CH₃, CH(CH₃)₂, CF₃, CHF₂, or CH₂F. In some cases, n is 1 and R⁴ is CH₃. In some cases, at least one R⁴ is C₀₋₃alkyleneCN. In some cases, at least one R⁴ is CN or CH₂CN. In some cases, n is 1 and R⁴ is CN or CH₂CN. In some cases, at least one R⁴ is C₁₋₃alkyleneOH or C₁₋₃alkylene-C₁₋₃alkoxy. In some cases, at least one R⁴ is CH₂OH, CH₂CH₂OH, OCH₃, CH₂OCH₃, or CH₂CH₂OCH₃. In some cases, n is 1 and R⁴ is CH₂OH, CH₂CH₂OH, CH₂OCH₃, or CH₂CH₂OCH₃. In some cases, at least one R⁴ is oxo. In some cases, at least one R⁴ is C₃₋₇spiro-cycloalkyl. In some cases, the spiro-cycloalkyl is spiro-cyclopropyl, spiro-cyclobutyl, or spiro-cyclopentyl. In some cases, the spiro-cycloalkyl is spiro-cyclopropyl or spiro-cyclobutyl. In some cases, at least one R⁴ is spiro-heterocycloalkyl having 4-7 total ring atoms and 1 or 2 heteroatoms independently selected from N, O and S. In some cases, the spiro-heterocycloalkyl is spiro-oxetanyl or spiro-tetrahydrofuranyl. In some cases, the spiro-heterocycloalkyl is spiro-oxetanyl. In some cases, at least one R⁴ is spiro-cyclopropyl, spiro-cyclobutyl, or spiro-oxetanyl. In some cases, each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl. In some cases, each R⁴ independently is CH₃, CH₂CH₃, CH₂CH₂CH₃, CH₂F, CN, CH₂CN, CH₂OH, CH₂CH₂OH, CH₂OCH₃, or spiro-cyclopropyl. In some cases,

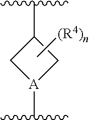

is

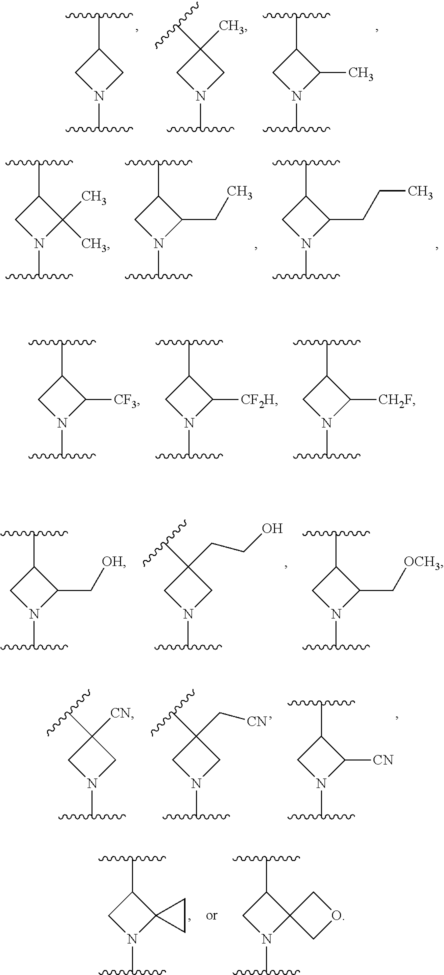

In some cases,

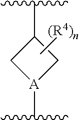

is

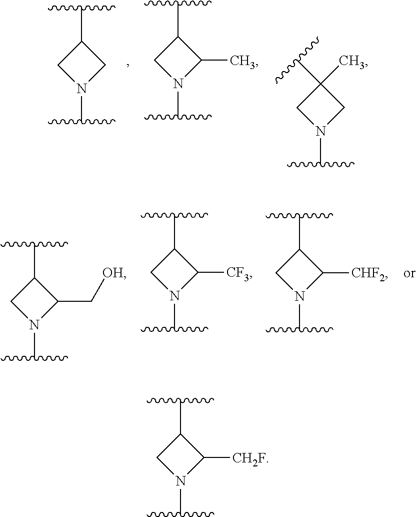

In some cases,

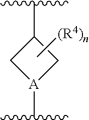

is

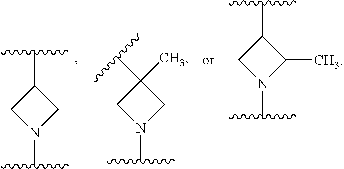 [SMILES: *C1CN(*)C1 ; *N1CC(*)(C)C1 ; *C1CN(*)C1C]

In some cases,

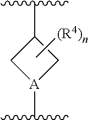

is

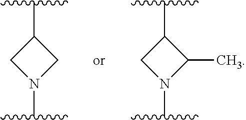 [SMILES: *C1CN(*)C1 ; *C1CN(*)C1C]

In some cases,

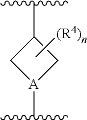

is

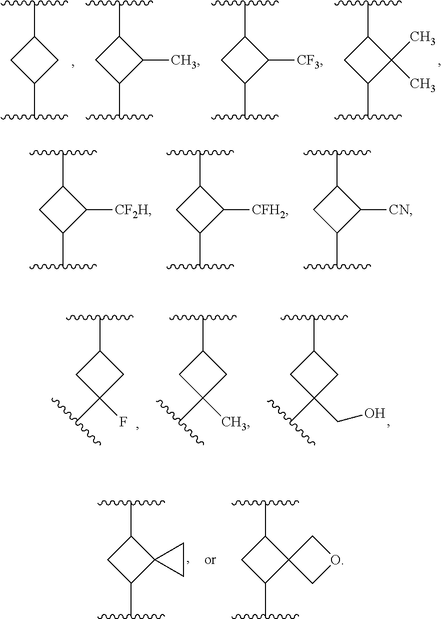

In some cases, W¹ is N. In some cases, W¹ is CH. In some cases, W¹ is C-halo or C—CN. In some cases. W¹ is C—F, C—Cl, or C—Br. In some cases, W¹ is C—F, C—Cl, or C—CN. In some cases, W¹ is C—C₁₋₃alkyl or C—C₁₋₃haloalkyl. In some cases, W¹ is C—CH₃, C—CH—CH₃, C₂CH₂CH₂CH₃, C—CH(CH₃)₂, C—CF₃, C—CHF₂, or C—CH₂F. In some cases. W¹ is C—CH₃, C—CH₂CH₃, C—CH₂F, C—CHF₂, or C—CF₃. In some cases, W¹ is C—CH; or C—CH₂CH₃. In some cases, W¹ is C—C₂₋₃alkenyl or C—C₂₋₃alkynyl, and each of the alkenyl and alkynyl is unsubstituted or substituted with 1 or more substituents. In some cases, each of the alkenyl and alkynyl is unsubstituted. In some cases, each of the alkenyl and alkynyl is substituted with 1-3 substituents (e.g., 1-3 substituents, 1-2 substituents, 1 substituent, 2 substituents, or 3 substituents). In some cases, each of the alkenyl and alkynyl is substituted with 1 substituent. In some cases, each of the alkenyl and alkynyl is substituted with 2 substituents. In some cases, each of the alkenyl and alkynyl is substituted with 3 substituents. In some cases, each substituent of the alkenyl and alkynyl independently is halo, C₁₋₃haloalkyl, C₀₋₃alkyleneOH, or C₀₋₃alkyleneC₁₋₄alkoxy. In some cases, W¹ is C—CH═CH₂, C—C(OH)═CH₂, C—CH═CH(OH), or C—CCH. In some cases, W¹ is C—C₀₋₃alkyleneOH or C—C₀₋₃alkylene-C₁₋₄alkoxy. In some cases, W¹ is C—OH, C—CH₂OH, C—CH₂CH₂OH, C—OCH₃, C—CH—OCH₃, or C—CH₂CH₂OCH₃. In some cases, W¹ is C—OH, C—CH₂OH, C—OCH₃, or C—CH₂OCH₃. In some cases, W¹ is CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, W¹ is CH, C—F, C—Cl, C—CN, C—CH₃, C—CH₂CH₃, C—OH, C—CH₂OH, C—OCH₃, or C—CH₂OCH₃. In some cases, W¹ is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃. In some cases, W² is N. In some cases, W² is CH. In some cases, W² is C-halo or C—CN. In some cases, W² is C—F, C—Cl, or C—Br. In some cases, W² is C—F, C—Cl, or C—CN. In some cases, W² is C—C₁₋₃alkyl or C—C₁₋₃haloalkyl. In some cases, W² is C—CH₃, C—CH₂CH₃, C—CH₂CH₂CH₃, C—CH(CH₃)₂, C—CF₃, C—CHF₂, or C—CH₂F. In some cases, W² is C—CH₃, C—CH₂CH₃, C—CH₂F, C—CHF₂, or C—CF₃. In some cases, W² is C—CH₃ or C—CH₂CH₃. In some cases, W² is C—C₂₋₃alkenyl or C—C₂₋₃alkynyl, and each of the alkenyl and alkynyl is unsubstituted or substituted with 1 or more substituents. In some cases, each of the alkenyl and alkynyl is unsubstituted. In some cases, each of the alkenyl and alkynyl is substituted with 1-3 substituents (e.g., 1-3 substituents, 1-2 substituents, 1 substituent, 2 substituents, or 3 substituents). In some cases, each of the alkenyl and alkynyl is substituted with 1 substituent. In some cases, each of the alkenyl and alkynyl is substituted with 2 substituents. In some cases, each of the alkenyl and alkynyl is substituted with 3 substituents. In some cases, each substituent independently is halo, C₁₋₃haloalkyl, C₀₋₃alkyleneOH, or C₀₋₃alkyleneC₁₋₄alkoxy. In some cases, W² is C—CH═CH₂, C—C(OH)═CH₂, C—CH═CH(OH), or C—CCH. In some cases. W² is C—C₀₋₃alkyleneOH or C—C₀₋₃alkylene-C₁₋₄alkoxy. In some cases, W² is C—OH, C—CH₂OH, C—CH₂CH₂OH, C—OCH₃, C—CH₂OCH₃, or C—CH₂CH₂OCH₃. In some cases, W² is C—OH, C—CH₂OH, C—OCH₃, or C—CH₂OCH₃. In some cases, W² is CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, W² is CH, C—F, C—Cl, C—CN, C—CH₃, C—CH₂CH₃, C—OH, C—CH₂OH, C—OCH₃, or C—CH₂OCH₃. In some cases, W² is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃. In some cases, each of W¹ and W² independently is N, CH, or C—CH₃. In some cases, W¹ is CH and W² is N, CH, or C—CH₃. In some cases, W² is N and W is N, CH, or C—CH₃. In some cases, W¹ is CH and W² is N. In some cases, X is N. In some cases, W¹ is CH and X is N. In some cases, W² is N and X is N. In some cases, W¹ is CH, W² is N, and X is N. In some cases, X is C—R^{5a}. In some cases, W¹ is CH and X is C—R^{5a}. In some cases, W² is N and X is C—R^{5a}. In some cases, W¹ is CH, W² is N, and X is C—R^{5a}. In some cases, W¹ is CH, C-halo, C—CN, C—C₁₋₃alkyl, C—C₁₋₃haloalkyl, C—C₀₋₃alkyleneOH, or C—C₀₋₃alkylene-C₁₋₄alkoxy; W² is N; and X is N or C—R^{5a}. In some cases, R^{5a} is H. In some cases, R^{5a} is CN. In some cases. R^{5a} is Br, Cl, or F. In some cases, R^{5a} is C₁₋₃alkyl or C₁₋₃haloalkyl. In some cases, R^{5a} is CH₃, CH₂CH₃, CF₃, CHF₂, or CH₂F. In some cases, R^{5a} is CH₃. In some cases, R^{5a} is C₂₋₆alkenyl or C₂₋₆alkynyl. In some cases, R^{5a} is C₂₋₃alkenyl or C₂₋₃alkynyl. In some cases. R^{5a} is

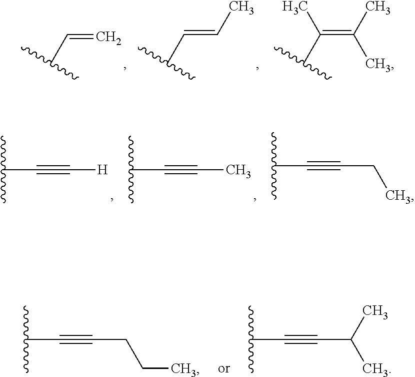

In some cases, R^{5a} is

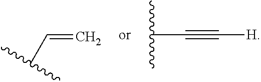

In some cases, R^{5a} is C₀₋₃alkyleneOH or C₀₋₃alkylene-C₁₋₃alkoxy. In some cases, R^{5a} is OH, CH₂OH, OCH₃, or CH₂OCH₃. In some cases, R^{5a} is C₃₋₅cycloalkyl. In some cases, R^{5a} is OH, CH₂OH, OCH₃, CH₂OCH₃, or

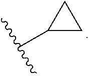 [SMILES: *C1CC1]

In some cases, R^{5a} is

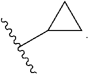 [SMILES: *C1CC1]

In some cases, R^{5a} is H, CN, Br, Cl, F, CH₃, CH₂CH₃, CF₃, CHF₂, CH₂F,

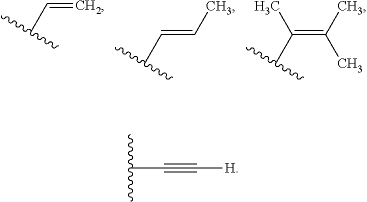

OH, CH₂OH, OCH₃, CH₂OCH₃, or

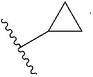 [SMILES: *C1CC1]

In some cases, R^{5a} is H, CN, or CH₃. In some cases, R^{5a} is CN or CH₃. In some cases, X is N, C—CH₃, or C—CN. In some cases, W¹ is CH; W² is N; and X is N, C—CH₃, or C—CN.

In some cases, R^{5b} is halo. In some cases, R^{5b} is Br, Cl, or F. In some cases, R^{5b} is C₁₋₃haloalkyl. In some cases, R^{5b} is CF₃, CF₂H, CFH₂, or CF₂CH₃. In some cases. R^{5b} is CF₃ or CF₂H. In some cases, R^{5b} is CF₃. In some cases. R^{5b} is CF₂H. In some cases, R^{5b} is CHF₂. In some cases, R^{5b} is C₁₋₃alkoxy or C₁₋₃thioalkyl. In some cases, R^{5b} is OCH₃, OCH₂CH₃, SCH₃, or SCH₂CH₃. In some cases, R^{5b} is OCH₃ or SCH₃. In some cases, R^{5b} is C₁₋₆alkyl, C₂₋₄alkenyl, or C₂₋₄alkynyl, each of which is unsubstituted or substituted with 1 or more substituents. In some cases, the C₁₋₆alkyl is CH₃, CH₂CH₃, CH₂CH₂CH₃, or CH(CH₃)₂, wherein each of the foregoing is unsubstituted or substituted with 1 or more substituents. In some cases, the C₂₋₄alkenyl is CH═CH₂ or CH═CHCH₃, wherein each of the foregoing is unsubstituted or substituted with 1 or more substituents. In some cases, the C₂₋₄ alkynyl is

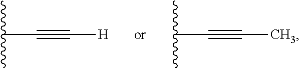

wherein each of the foregoing is unsubstituted or substituted with 1 or more substituents. In some cases, the C₁₋₃alkyl, C₂₋₄alkenyl, and C₂₋₄alkynyl is unsubstituted. In some cases. R^{5b} is CH₃, CH₂CH₃, CH₂CH₂CH₃, or CH(CH₃)₂. In some cases, the C₁₋₆alkyl, C₂₋₄alkenyl, and C₂₋₄alkynyl is substituted with 1-3 substituents (e.g., 1-3 substituents, 1-2 substituents, 1 substituent, 2 substituents, or 3 substituents). In some cases, each of the C₁₋₆alkyl, C₂₋₄alkenyl, and C₂₋₄alkynyl is substituted with 1 substituent. In some cases, each of the C₁₋₆alkyl, C₂₋₄alkenyl, and C₂₋₄alkynyl is substituted with 2 substituents. In some cases, each of the C₁₋₆alkyl, C₂₋₄alkenyl, and C₂₋₄alkynyl is substituted with 3 substituents. In some cases, each substituent of the alkyl, alkenyl, and alkynyl independently is C₁₋₃haloalkyl, C₀₋₆alkylene(OH), C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 3-7 total ring atoms and 1-3 heteroatoms independently selected from N, O, and S, or phenyl. In some cases, each of the substituents independently is CH₃, CF₃, CF₂H, CFH₂, OH, OCH₃, OCF₃, CH₂OH, CH₂OCH₃, cyclopropyl, cyclobutyl, or phenyl. In some cases, R^{5b} is CH₃, CH₂CH₃, CH₂CH₂CH₃, CH(CH₃)₂,

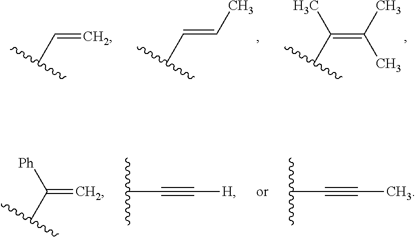

In some cases, R^{5b} is C₃₋₇cycloalkyl, C₅₋₇cycloalkenyl, heterocycloalkyl having 3-7 total ring atoms and 1-3 heteroatoms independently selected from N, O, and S, or heterocycloalkenyl having 5-7 total ring atoms and 1-3 heteroatoms independently selected from N, O, and S, wherein each of the foregoing is unsubstituted or substituted with 1 or more substituents. In some cases, the cycloalkyl, cycloalkenyl, heterocycloalkyl, and heterocycloalkenyl are each substituted with 1-3 substituents (e.g., 1-3 substituents, 1-2 substituents, 1 substituent, 2 substituents, or 3 substituents). In some cases, each substituent of the cycloalkyl, cycloalkenyl, heterocycloalkyl, and heterocycloalkenyl independently is halo, C₁₋₃alkyl, C₁₋₃haloalkyl, C₀₋₆alkylene (OH), or C₀₋₃alkylene-C₁₋₃alkoxy. In some cases, the C₃₋₇cycloalkyl is cyclopropyl, cyclobutyl, cyclopentyl, or cyclohexyl, wherein each of the foregoing is unsubstituted or substituted with 1-3 substituents (e.g., 1-3 substituents, 1-2 substituents, 1 substituent, 2 substituents, or 3 substituents). In some cases, the C₅₋₇cycloalkenyl is cyclopentenyl or cyclohexenyl, wherein each of the foregoing is unsubstituted or substituted with 1-3 substituents (e.g., 1-3 substituents. 1-2 substituents, 1 substituent, 2 substituents, or 3 substituents). In some cases, theterocycloalkyl having 3-7 total ring atoms and 1-3 heteroatoms independently selected from N, O, and S is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothipyranyl, dithianyl, morpholinyl, or thiomorpholinyl, wherein each of the foregoing is unsubstituted or substituted with 1-3 substituents (e.g., 1-3 substituents, 1-2 substituents, 1 substituent, 2 substituents, or 3 substituents). In some cases, theterocycloalkenyl having 5-7 total ring atoms and 1-3 heteroatoms independently selected from N, O, and S is dihydropyrrolyl, dihydrofuranyl, dihydrothiopheneyl, dihydroisoxazolyl, tetrahydropyridinyl, dihydropyranyl, or dihydrothipyranyl, wherein each of the foregoing is unsubstituted or substituted with 1-3 substituents (e.g., 1-3 substituents, 1-2 substituents, 1 substituent, 2 substituents, or 3 substituents). In some cases, R^{5b} is cyclopropyl, cyclobutyl, cyclopentenyl, oxetanyl, or tetrahydrofuranyl. In some cases, R^{5b} CH₃, CF₃, CF₂H, CFH₂, CH₂CH₃, CH₂CH₂CH₃, CH(CH₃)₂,

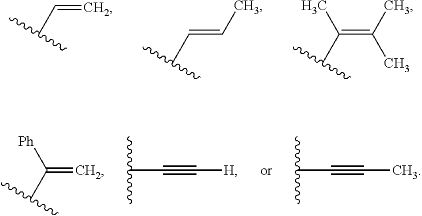

In some cases, R^{5b} is Br, Cl, F, OCH₃, SCH₃, CH₃, CH₂CH₃, CH₂CH₂CH₃, CH(CH₃)₂,

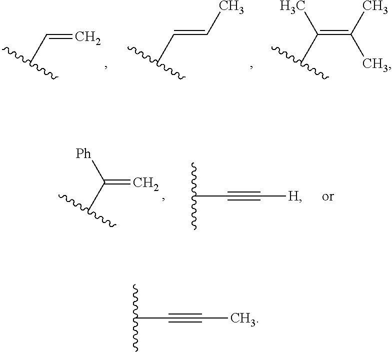

In some cases,

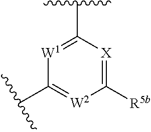

is

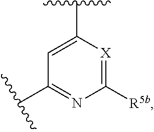

X is N, C—CH₃, or C—CN; and R^{b} is CF₃ or CF₂H. In some cases,

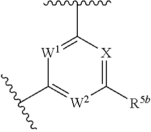

is

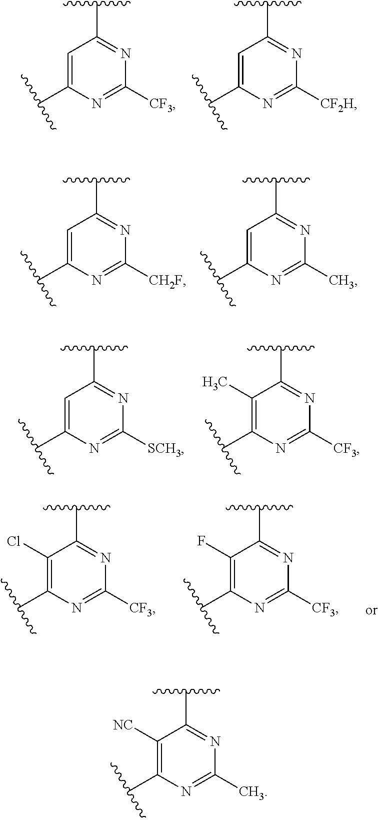

In some cases,

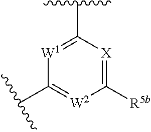

is

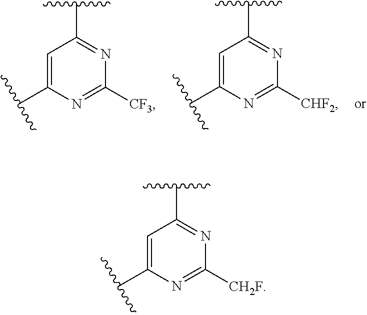

In some cases,

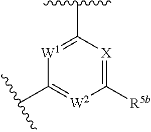

is

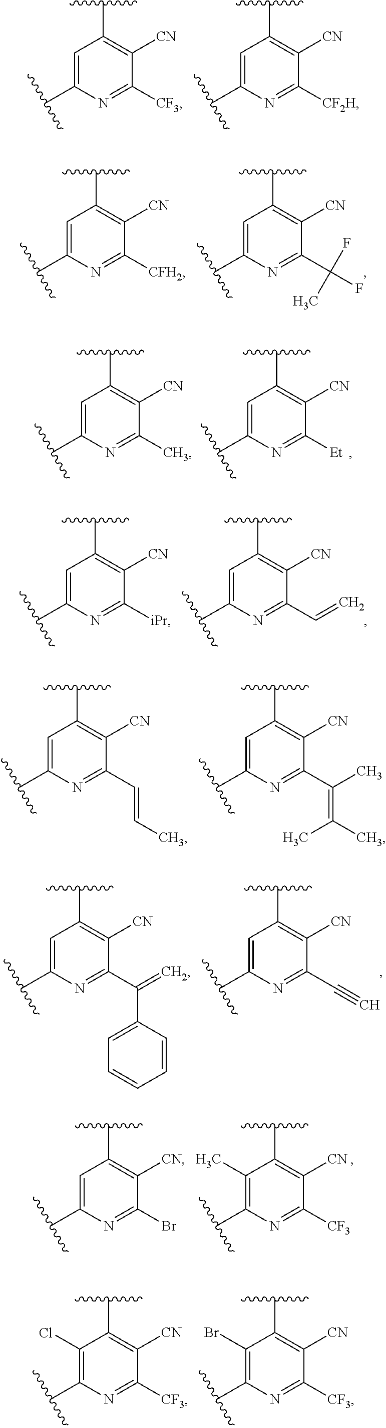

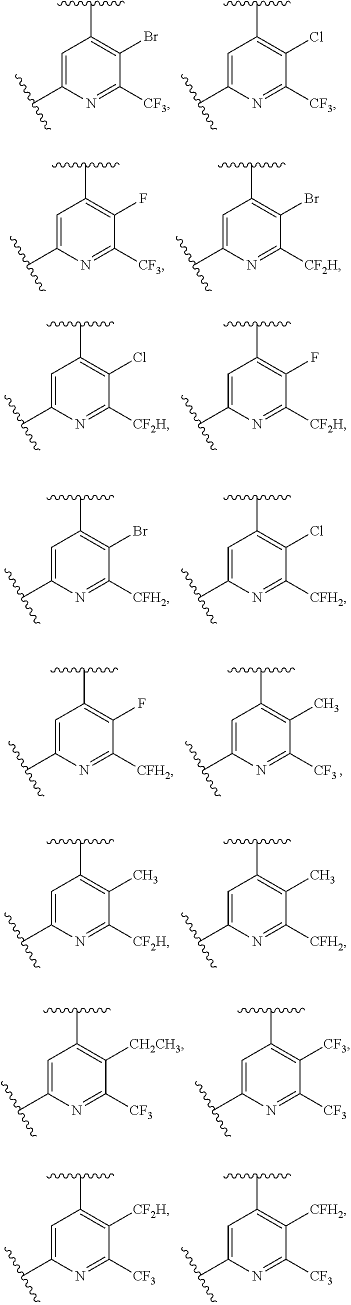

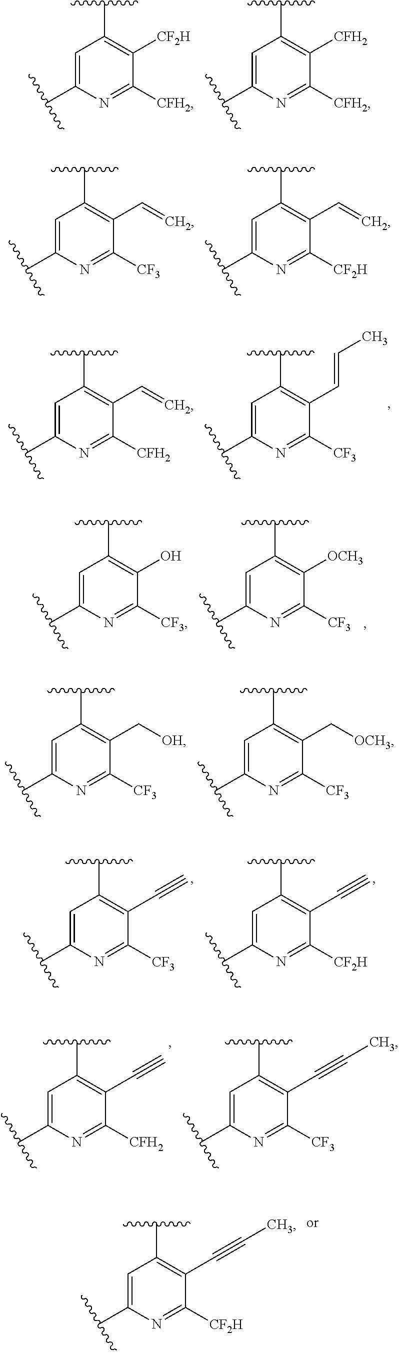

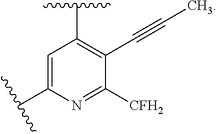

In some cases,

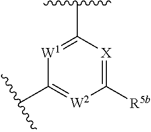

is

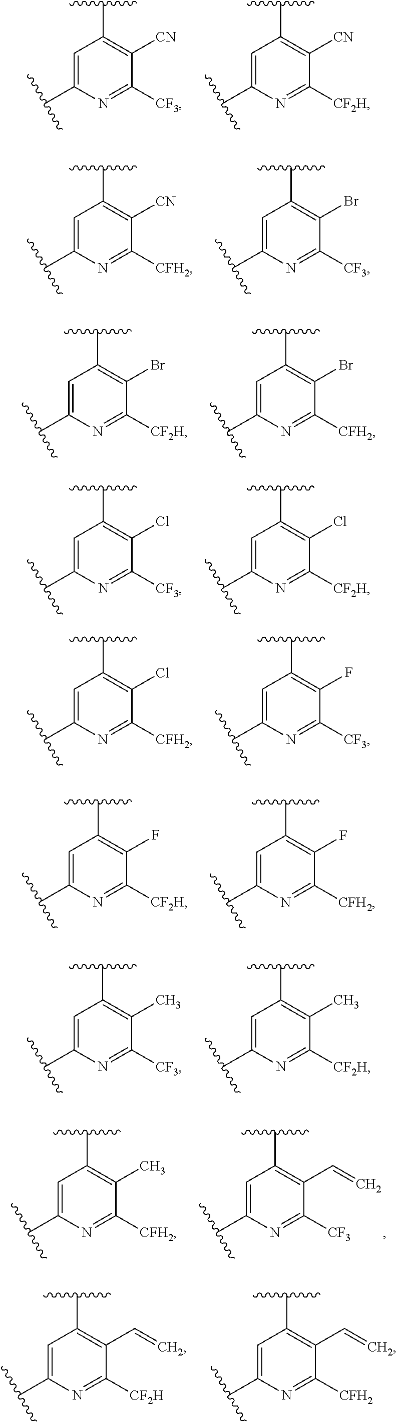

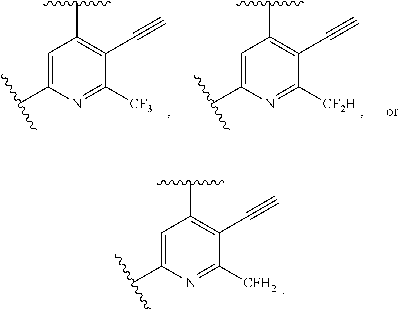

In some cases,

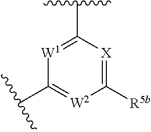

is

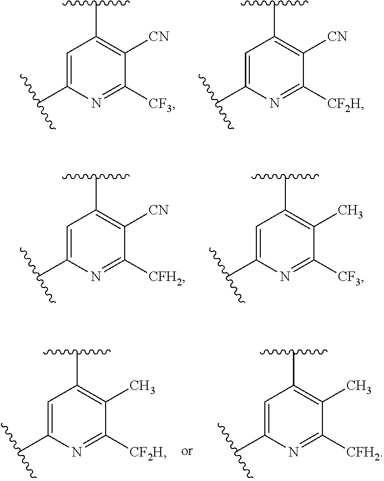

In some cases,

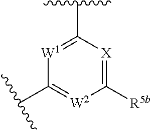

is

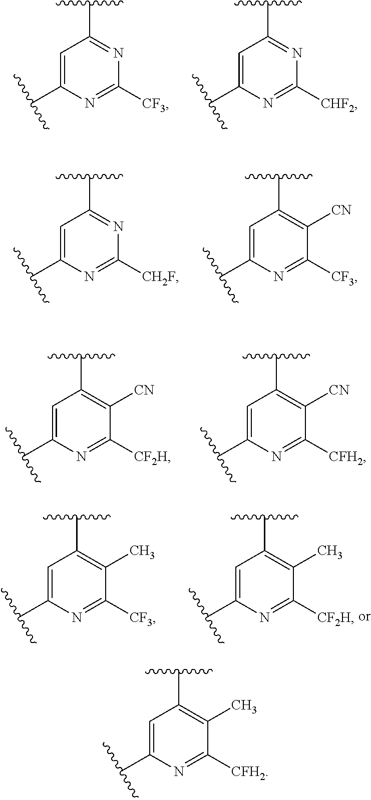

In some cases, q is 0. In some cases, q is 1. In some cases, q is 2. In some cases, r is 0. In some cases, r is 1. In some cases, each of q and r is 0. In some cases, q is 0 and r is 1. In some cases, q is 1 and r is 0. In some cases, q is 1 and r is 1. In some cases, q is 2 and r is 0. In some cases, q is 2 and r is 1. In some cases,

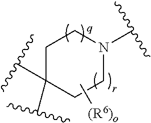

is

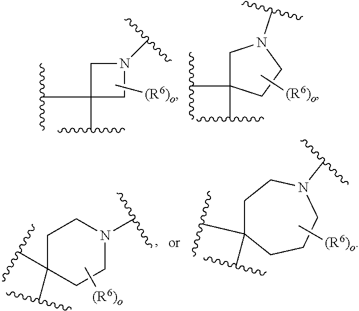

In some cases,

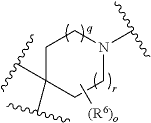

is

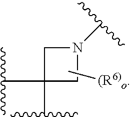

In some cases,

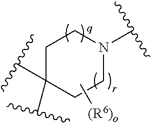

is

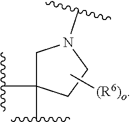

In some cases,

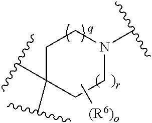

is

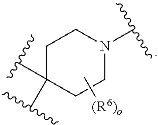

In some cases,

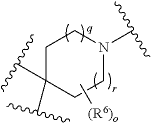

is

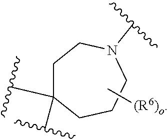

In some cases, the compound of Formula (II) has a structure of Formula (IIA):

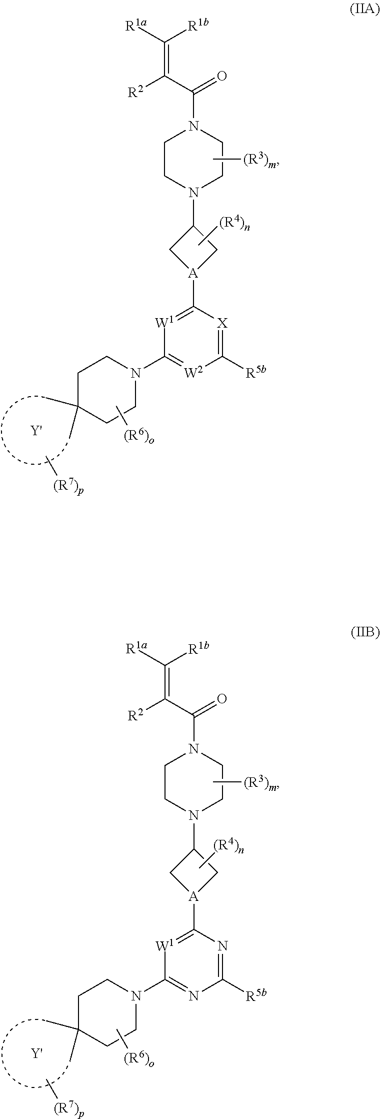

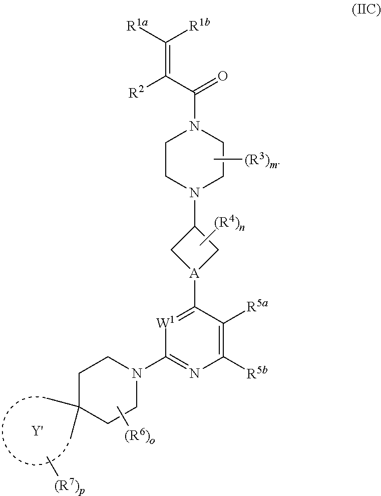

In some cases, the compound is a compound of Formula (IIA), or a pharmaceutically acceptable salt thereof. In some cases, the compound is a compound of Formula (IIA), wherein W¹ is CH, C-halo, C—CN, C—C₁₋₃alkyl, C—C₁₋₃haloalkyl, C—C₀₋₃alkyleneOH, or C—C₀₋₃alkylene-C₁₋₄alkoxy; W² is N; and X is N or C—R^{5a}, or a pharmaceutically acceptable salt thereof. In some cases, the compound is a compound of Formula (IIB), or a pharmaceutically acceptable salt thereof. In some cases, the compound is a compound of Formula (IIC), or a pharmaceutically acceptable salt thereof.

In some cases, o is 0. In some cases, o is 1. In some cases, o is 2. In some cases, o is 3. In some cases, o is 4. In some cases, at least one R⁶ is halo or CN. In some cases, at least one R⁶ is Br, Cl, F, or CN. In some cases, at least one R⁶ is F. In some cases, o is 1 or 2 and each Re independently is F. In some cases, at least one R⁶ is C₁₋₃alkyl or C₁₋₃haloalkyl. In some cases, at least one R⁶ is CH₃, CH₂F, CHF₂ or CF₃. In some cases, o is 1 or 2 and each Re independently is CH₃. In some cases, at least one R⁶ is C₀₋₃alkyleneOH, C₀₋₃alkylene-C₁₋₃alkoxy, deuterated C₀₋₃alky lene-C₁₋₃alkoxy, or C₁₋₄alkylene-N(R^{N1})₂, and each R^{N1} independently is H or CH₃. In some cases, each R^{N1} independently is H. In some cases, at least one R⁶ is OH, CH₂OH, CH₂CH₂OH, OCH₃, OCD₃, CH₂OCH₃, or CH₂CH₂OCH₃. In some cases, at least one R⁶ is CH₂N(CH₃)₂, CH₂NH(CH₃), or CH₂NH₂. In some cases, at least one R⁶ is OH, CH₂OH, OCH₃, OCD₃, CH₂OCH₃, or CH₂N(CH₃)₂. In some cases, o is 1 and R⁶ is OH, CH₂OH, OCH₃, or CH₂OCH₃. In some cases, at least one R⁶ is oxo or ═CH₂. In some cases, at least one R⁶ is C₃₋₇cycloalkyl, C₄₋₇cycloalkenyl, heterocycloalkyl having 4-7 total ring atoms and 1 or 2 heteroatoms independently selected from N. O and S, heterocycloalkenyl having 4-7 total ring atoms and 1 or 2 heteroatoms independently selected from N, O and S; wherein each of the foregoing is unsubstituted or substituted with 1 or more substituents. In some cases, at least one R⁶ is cyclopropyl, cyclobutyl, oxetanyl, or tetrahydrofuranyl; wherein each of the foregoing is unsubstituted or substituted with 1 or more substituents. In some cases, at least one R⁶ is cyclopropyl. In some cases, at least one R⁶ is spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S, wherein each of the foregoing is unsubstituted or substituted with 1 or more substituents. In some cases, at least one R⁶ is spiro-C₃₋₇cycloalkyl or spiro-heterocycloalkyl having 4-7 total ring atoms and 1 or 2 heteroatoms independently selected from N, O and S, wherein each of the foregoing is unsubstituted or substituted with 1 or more substituents. In some cases, at least one R⁶ is spiro-cyclopropyl, spiro-cyclobutyl, spiro-oxetanyl, or spiro-tetrahydrofuranyl, wherein each of the foregoing is unsubstituted or substituted with 1 or more substituents. In some cases, at least one R⁶ is spiro-cyclopropyl that is unsubstituted or substituted with 1 or more substituents. In some cases, at least one R⁶ is spiro-cyclopropyl. In some cases, two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; wherein each of the foregoing is unsubstituted or substituted with 1 or more substituents. In some cases, the fused-C₃₋₇cycloalkyl is fused-cyclopropyl, fused-cyclobutyl, or fused-cyclopentyl, wherein each of the foregoing is unsubstituted or substituted with 1 or more substituents. In some cases, two vicinal Re, together with the atoms to which they are attached, form fused-cyclopropyl, fused-cyclobutyl, fused-cyclopentyl, fused-oxetanyl, or fused-tetrahydrofuranyl, wherein each of the foregoing is unsubstituted or substituted with 1 or more substituents. In some cases, two vicinal R⁶, together with the atoms to which they are attached, form fused-cyclopropyl, fused-cyclobutyl, or fused-cyclopentyl, wherein each of the foregoing is unsubstituted or substituted with 1 or more substituents. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is unsubstituted. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1 or more substituents. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1-4 substituents (e.g., 1-4 substituents, 1-3 substituents, 1-2 substituents, 1 substituent, 2 substituents, 3 substituents, or 4 substituents). In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1 or 2 substituents. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1 substituent. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 2 substituents. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 3 substituents. In some cases, the cycloalkyl, cycloalkenyl, heterocycloalkyl, heterocycloalkenyl, spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 4 substituents. In some cases, each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN. In some cases, each substituent independently is halo, OH, C₁₋₃alkoxy, or CN. In some cases, each substituent independently is F, Cl, OH, OCH₃, OCH₂CH₃, or CN. In some cases, two non-neighboring R⁶ join together to form a C₁₋₃alkylene bridge, a C₂₋₃alkenylene bridge, a C₁₋₃ether bridge, or a C₁₋₃thioether bridge. In some cases, two non-neighboring R⁶ join together to form a C₁₋₃alkylene bridge, a C₂₋₃alkenylene bridge, or a C₁₋₃ether bridge. In some cases, two non-neighboring R⁶ join together to form a C₁₋₃alkylene bridge or a C₂₋₃alkenylene bridge. In some cases, two non-neighboring R⁶ join together to form a C₁₋₃ether bridge or a C₁₋₃thioether bridge. In some cases, two non-neighboring R⁶ join together to form a C₁alkylene bridge

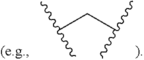 [SMILES: *C*]

In some cases, two non-neighboring R⁶ join together to form a C₂alkylene bridge

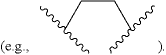 [SMILES: *CC*]

In some cases, two non-neighboring R⁶ join together to form a C₃alkylene bridge

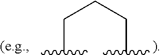 [SMILES: *CCC*]

In some cases, two non-neighboring R⁶ join together to form a C₂alkenylene bridge

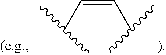 [SMILES: */C=C\*]

In some cases, two non-neighboring R⁶ join together to form a C₃alkenylene bridge

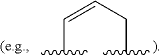 [SMILES: */C=C\C*]

In some cases, two non-neighboring R⁶ join together to form a C₁₋₃ether bridge

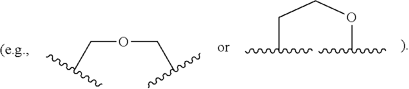 [SMILES: *COC* ; *CCO*]

In some cases, two non-neighboring R⁶ join together to form a C₁₋₃thioether bridge

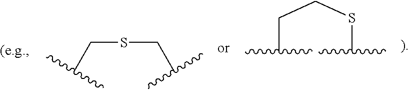 [SMILES: *CSC* ; *CCS*]

In some cases, two non-neighboring R⁶ join together to form —CH₂—, —CH₂CH₂—, —CH₂CH₂CH₂—, —CH₂—CH═CH— or —CH₂OCH₂—. In some cases, at least one R⁶ is Br, Cl, F, CN, CH₃, CH₂F, CHF₂, CF₃, OH, CH₂OH, OCH₃, OCD₃, CH₂OCH₃, CH₂N(CH₃)₂, oxo, ═CH₂, or cyclopropyl, or two vicinal R⁶, together with the atoms to which they are attached, form fused-cyclopropyl, fused-cyclobutyl, or fused-cyclopentyl, or two non-neighboring R⁶ join together to form —CH₂—, —CH₂CH₂—, —CH₂CH₂CH₂—, —CH₂—CH═CH— or —CH₂OCH₂—; wherein each of the foregoing cycloalkyl groups is unsubstituted or substituted with 1-4 substituents, and each substituent independently is F, Cl, OH, OCH₃, OCH₂CH₃, or CN. In some cases, o is 0; or o is 1 and R⁶ is H, CH₃, or cyclopropyl. In some cases, o is 0; or o is 1 and R⁶ is CH₃. In some cases,

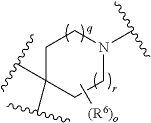

is

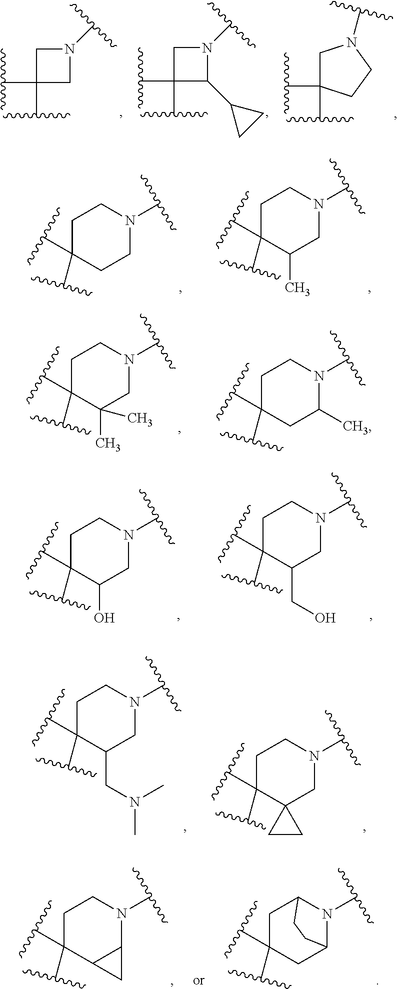

In some cases,

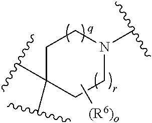

is

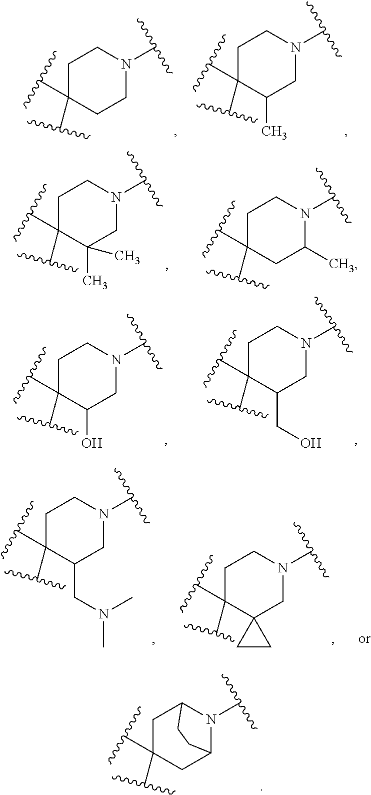

In some cases,

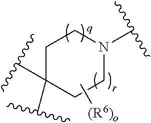

is

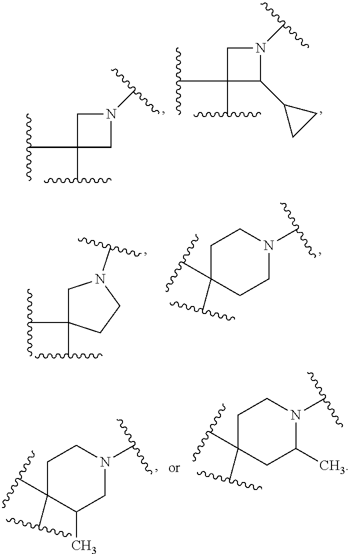

In some cases, is

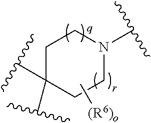

is

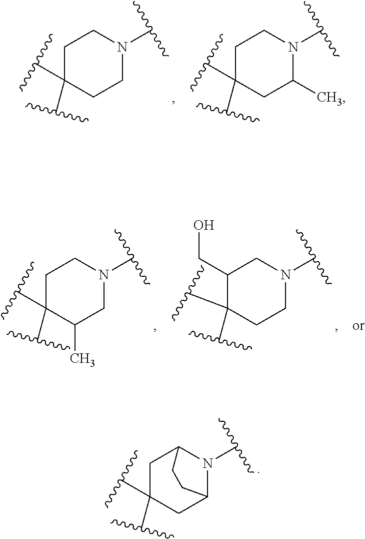

In some cases,

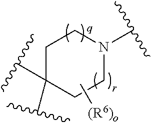

is

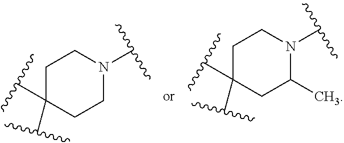 [SMILES: *N1CCC(*)(*)CC1 ; *N1CCC(*)(*)CC1C]

In some cases,

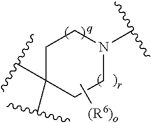

is

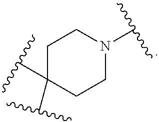 [SMILES: *N1CCC(*)(*)CC1]

In some cases,

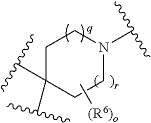

is

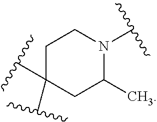 [SMILES: *N1CCC(*)(*)CC1C]

In some cases, Y′ is C₃₋₇cycloalkyl or C₄₋₇cycloalkenyl. In some cases, Y′ is cyclopropyl, cyclobutyl, cyclopentyl, cyclohexyl, cyclopentenyl, or cyclohexenyl. In some cases, Y′ is cyclopropyl, cyclobutyl, cyclopentyl, or cyclopentenyl. In some cases, Y′ is cyclopropyl or cyclobutyl. In some cases, Y′ is heterocycloalkyl having 3 to 7 total ring atoms and 1-3 heteroatoms independently selected from N, O, and S. In some cases, Y′ is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, dioxolanyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothiopyranyl, ditbianyl, morpholinyl, or thiomorpholinyl. In some cases, Y′ is oxetanyl, thietanyl, tetrahydrofuranyl, dioxolanyl, oxazolidinyl, tetrahydropyranyl, tetrahydrothiopyranyl, or morpholinyl. In some cases, Y′ is oxetanyl, pyrrolidinyl, tetrahydrofuranyl, dioxolanyl, oxazolidinyl, isoxazolidinyl, or tetrahydropyranyl. In some cases, Y′ is tetrahydrofuranyl, tetrahydropyranyl, or morpholinyl. In some cases, Y′ is tetrahydrofuranyl or tetrahydropyranyl. In some cases, Y′ is heterocycloalkenyl having 5 to 7 total ring atoms and 1-3 heteroatoms independently selected from N, O, and S. In some cases, Y′ is dihydropyrrolyl, dihydrofuranyl, dihydrothiopheneyl, dihydroisoxazolyl, tetrahydropyridinyl, dihydropyranyl, or dihydrothipyranyl. In some cases, Y′ is dibydropyrrolyl, dihydrofuranyl or dihydropyranyl. In some cases, Y′ is dihydrofuranyl or dihydropyranyl. In some cases, Y′ is cyclopropyl, cyclobutyl, cyclopentyl, cyclohexyl, cyclopentenyl, cyclohexenyl, aziridinyl, oxiranyl, thiranyl, azetidinyl, oxetanyl, thietanyl, pyrrolidinyl, imidazolidinyl, pyrazolidinyl, tetrahydrofuranyl, tetrahydrothiopheneyl, dioxolanyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothiopyranyl, dithianyl, morpholinyl, thiomorpholinyl, dihydropyrrolyl, dihydrofuranyl, dihydrothiophencyl, dihydroisoxazolyl, tetrahydropyridinyl, dihydropyranyl, or dihydrothipyranyl. In some cases, Y′ is oxetanyl, thietanyl, tetrahydrofuranyl, dioxolanyl, oxazolidinyl, tetrahydropyranyl, tetrahydrothiopyranyl, morpholinyl, dihydrofuranyl or dihydropyranyl. In some cases, Y′ is tetrahydrofuranyl, tetrahydropyranyl, morpholinyl, dihydrofuranyl or dihydropyranyl. In some cases, Y′ is cyclopropyl, cyclobutyl, cyclopentyl, cyclopentenyl, oxetanyl, pyrrolidinyl, tetrahydrofuranyl, dioxolanyl, oxazolidinyl, isoxazolidinyl, tetrahydropyranyl, dihydropyrrolyl, dihydrofuranyl or dihydropyranyl. In some cases, p is 0. In some cases, p is 1. In some cases, p is 2. In some cases, p is 3. In some cases, p is 4. In some cases, p is 5. In some cases, at least one R⁷ is F, Cl, Br, CN, or CH₂CN. In some cases, at least one R⁷ is F, Cl, or Br. In some cases, at least one R⁷ is CN or CH₂CN. In some cases, at least one R⁷ is F or CH₂CN, In some cases, at least one R⁷ is C₁₋₃alkyl, C₁₋₃haloalkyl, or C₂₋₃alkenyl. In some cases, at least one R⁷ is CH₃, CH₂CH₃, CH₂F, CHF₂, or CF₃, In some cases, at least one R⁷ is CH₃, CH₂F, or CHF₂. In some cases, at least one R⁷ is C₀₋₃alkyleneOH or C₀₋₃alkylene-C₁₋₃alkoxy. In some cases, at least one R⁷ is OH, OCH₃, OCH₂CH₃, CH₂OH, CH₂OCH₃, or CH₂CH₂OCH₃. In some cases, at least one R⁷ is OH or CH₂OCH₃. In some cases, at least one R⁷ is C(═O)OC₁₋₃alkyl. In some cases, at least one R⁷ is C(═O)OCH₃. In some cases, at least one R⁷ is oxo or ═CH₂. In some cases, at least one R⁷ is C₀₋₄alkylene-C₃₋₇cycloalkyl; C₀₋₄alkylene-heterocycloalkyl having 3-7 total ring atoms and 1-3 heteroatoms independently selected from N, O, and S; C₀₋₄alkylene-C₆₋₁₀aryl; or C₀₋₄alkylene-heteroaryl having 4-7 total ring atoms and 1-3 heteroatoms independently selected from N, O, and S; wherein each of the foregoing is unsubstituted or substituted with 1 or more substituents. In some cases, at least one R⁷ is C₀₋₄alkylene-cyclopropyl, C₀₋₄alkylene-cyclobutyl, C₀₋₄alkylene-cyclopentyl, C₀₋₁alkylene-oxetanyl, C₀₋₄alkylene-pyrrolidinyl, C₀₋₄alkylene-phenyl, or C₀₋₄alkylene-oxadiazolyl; wherein each of the foregoing is unsubstituted or substituted with 1 or more substituents. In some cases, at least one R⁷ is cyclopropyl. In some cases, at least one R₇ is spiro-C₃₋₇cycloalkyl or a spiro-heterocycloalkyl ring having 4-7 total ring atoms and 1 or 2 heteroatoms independently selected from N, O and S; wherein each of the foregoing is unsubstituted or substituted with 1 or more substituents. In some cases, at least one R′ is spiro-cyclopropyl, spiro-cyclobutyl, spiro-oxetanyl, spiro-tetrahydrofuranyl, or spiro-tetrahydropyranyl, wherein each of the foregoing is unsubstituted or substituted with 1 or more substituents. In some cases, at least one R′ is spiro-cyclopropyl, spiro-cyclobutyl, spiro-oxetanyl, spiro-tetrahydrofuranyl, or spiro-tetrahydropyranyl, wherein each of the foregoing is unsubstituted or substituted with 1 or more substituents. In some cases, two vicinal R⁷, together with the atoms to which they are attached, form fused-C₃₋₇cycloalkyl or fused-heterocycloalkyl having 3-7 total ring atoms and 1-3 heteroatoms independently selected from N, O, and S; wherein each of the foregoing is unsubstituted or substituted with 1 or more substituents. In some cases, two vicinal R′, together with the atoms to which they are attached, form fused-cyclopropyl, fused-cyclobutyl, fused-oxetanyl, or fused-tetrahydrofuranyl. In some cases, two vicinal R⁷, together with the atoms to which they are attached, form fused-cyclopropyl or fused-cyclobutyl. In some cases, two vicinal R⁷, together with the atoms to which they are attached, form fused-tetrahydrofuranyl. In some cases, two vicinal R⁷, together with the atoms to which they are attached, form fused-C₆₋₁₀aryl or fused-heteroaryl having 5-7 total ring atoms and 1-3 heteroatoms independently selected from N, O, and S; wherein each of the foregoing is unsubstituted or substituted with 1 or more substituents. In some cases, two vicinal R⁷, together with the atoms to which they are attached, form fused-phenyl, fused-thiophenyl, fused-pyrazolyl, fused-thiazolyl, fused-pyridyl, fused-pyridazinyl, fused-pyrazinyl, or fused pyrimidinyl; wherein each of the foregoing is unsubstituted or substituted with 1 or more substituents. In some cases, two vicinal R⁷, together with the atoms to which they are attached, form fused-phenyl, fused-thiophenyl, fused-pyrazolyl, fused-thiazolyl, fused-pyridyl, fused-pyridazinyl, or fused-pyrazinyl; wherein each of the foregoing is unsubstituted or substituted with 1 or more substituents. In some cases, two vicinal R⁷, together with the atoms to which they are attached, form fused-pyrazolyl, fused-pyridyl, fused-pyridazinyl, or fused-pyrimidinyl; wherein each of the foregoing is unsubstituted or substituted with 1 or more substituents. In some cases, each of the foregoing cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl is unsubstituted or substituted with 1-4 substituents. In some cases, each of the foregoing cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl is unsubstituted. In some cases, the cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl of any of the foregoing is substituted with 1-4 substituents (e.g., 1-4 substituents, 1-3 substituents, 1-2 substituents, 1 substituent, 2 substituents, 3 substituents, or 4 substituents). In some cases, the cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl of any of the foregoing is substituted with 1 or 2 substituents. In some cases, the cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl of any of the foregoing is substituted with 1 substituent. In some cases, the cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl of any of the foregoing is substituted with 2 substituents. In some cases, the cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl of any of the foregoing is substituted with 3 substituents. In some cases, the cycloalkyl, heterocycloalkyl, aryl, heteroaryl, spiro-cycloalkyl, spiro-heterocycloalkyl, fused-cycloalkyl, fused-heterocycloalkyl, fused-aryl, and fused-heteroaryl of any of the foregoing is substituted with 4 substituents. In some cases, each substituent of the foregoing independently is halo, CN, oxo, or C₁₋₃alkyl. In some cases, each substituent independently is F, Cl, Br, CN, oxo, or CH₃. In some cases, each R⁷ independently is F, Cl, Br, CN, CH₂CN, CH₃, CH₂CH₃, CH₂F, CHF₂, CF₃, OH, OCH₃, OCH₂CH₃, CH₂OH, CH₂OCH₃, CH₂CH₂OCH₃, C(═O)OCH₃, oxo, ═CH₂, cyclopropyl, spiro-cyclopropyl, spiro-cyclobutyl, spiro-oxetanyl, spiro-tetrahydrofuranyl, spiro-tetrahydropyranyl; or two R⁷, together with the atoms to which they are attached, form fused-cyclopropyl, fused-cyclobutyl, fused-oxetanyl, fused-tetrahydrofuranyl, fused-phenyl, fused-thiophenyl, fused-pyrazolyl, fused-thiazolyl, fused-pyridyl, fused-pyridazinyl, fused-pyrazinyl, or fused-pyrimidinyl; wherein each of the pyrazolyl, pyridyl, pyridazinyl, and pyrimidinyl is unsubstituted or substituted with CH₃. In some cases, each substituent independently is F, Cl, Br, CN, oxo, or CH₃. In some cases, each R′ independently is F, Cl, Br, CN, CH₂CN, CH₃, CH₂CH₃, CH₂F, CHF₂, CF₃, OH, OCH₃, OCH₂CH₃, CH₂OH, CH₂OCH₃, CH₂CH₂OCH₃, C(═O)OCH₃, oxo, ═CH₂, cyclopropyl, spiro-cyclopropyl, spiro-cyclobutyl, spiro-oxetanyl, or spiro-tetrahydrofuranyl; or two R⁷, together with the atoms to which they are attached, form fused-cyclopropyl, fused-cyclobutyl, fused-oxetanyl, fused-tetrahydrofuranyl, fused-phenyl, fused-thiophenyl, fused-pyrazolyl, fused-thiazolyl, fused-pyridyl, fused-pyridazinyl, or fused-pyrazinyl; wherein each of the pyrazolyl, pyridyl, and pyridazinyl is substituted with CH₃. In some cases, Y′ is substituted with F, CH₂CN, CH₃, CH₂F, CHF₂, OH, CH₂OCH₃, oxo,

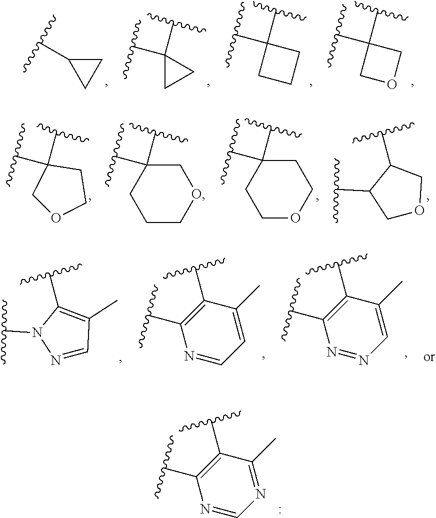

or a combination of any of the foregoing. In some cases. Y′ is substituted with F, CH₂CN, CH₃, CH₂F, CHF₂, OH, CH₂OCH₃, oxo,

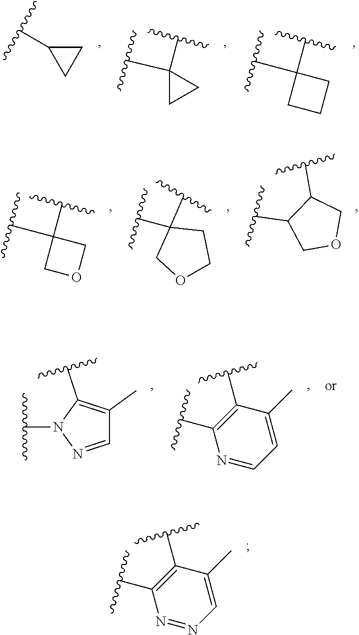

or a combination of any of the foregoing. In some cases,

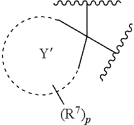

is

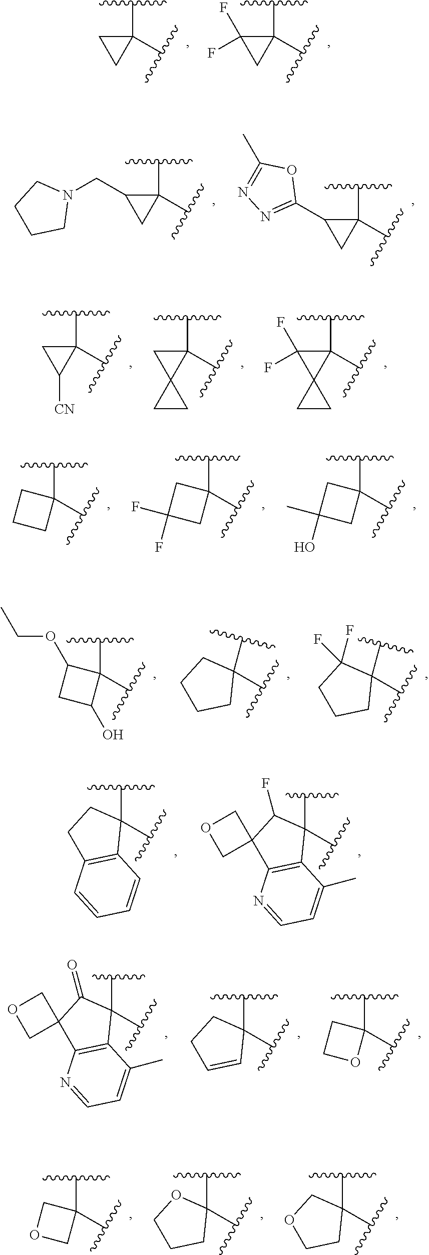

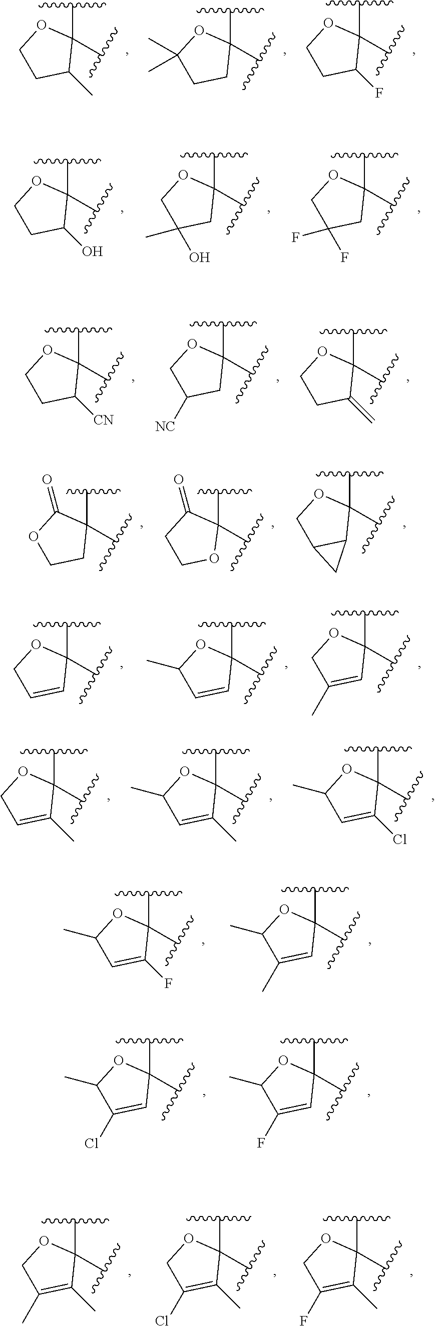

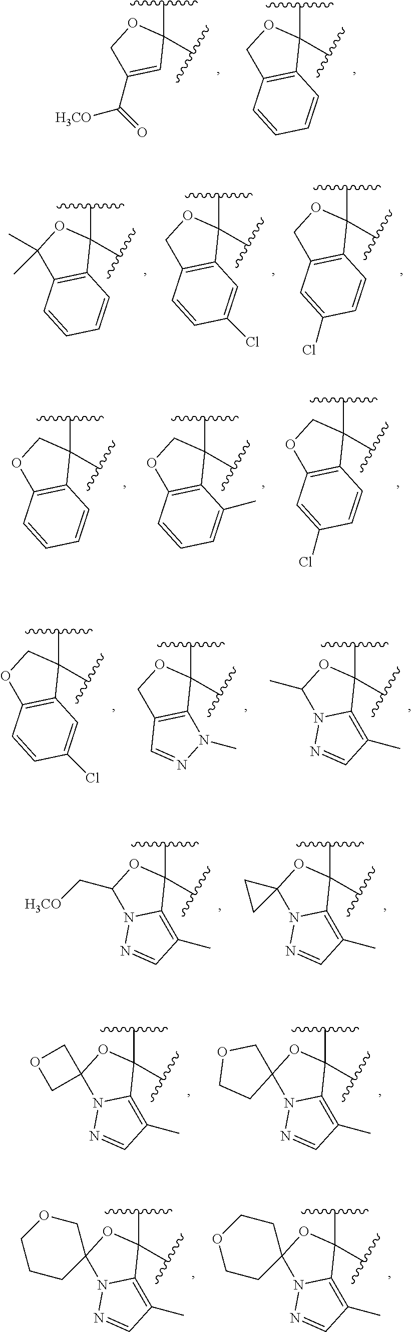

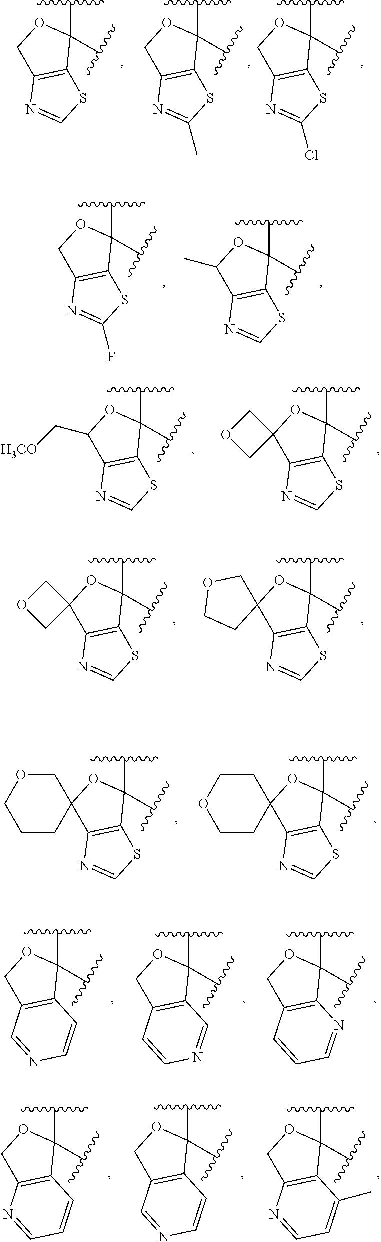

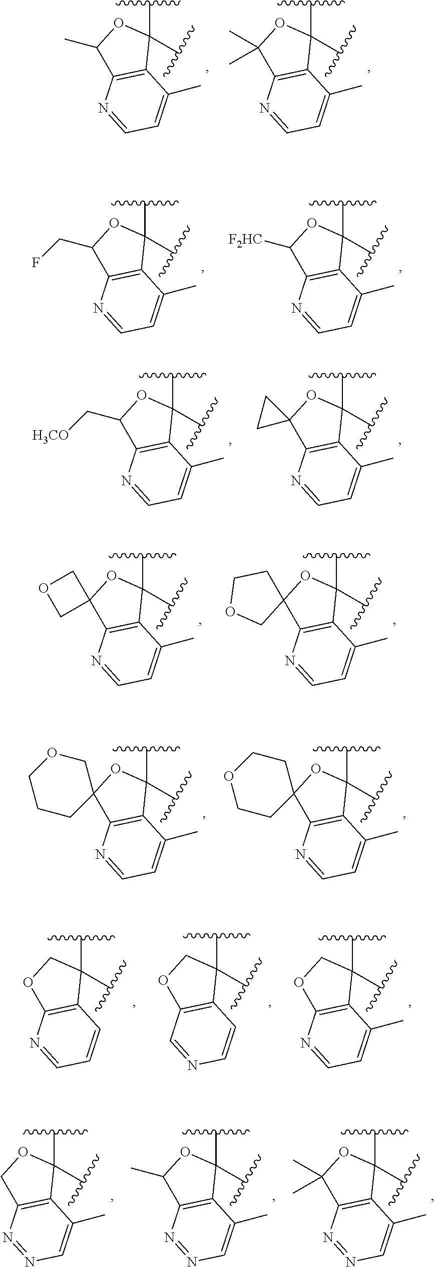

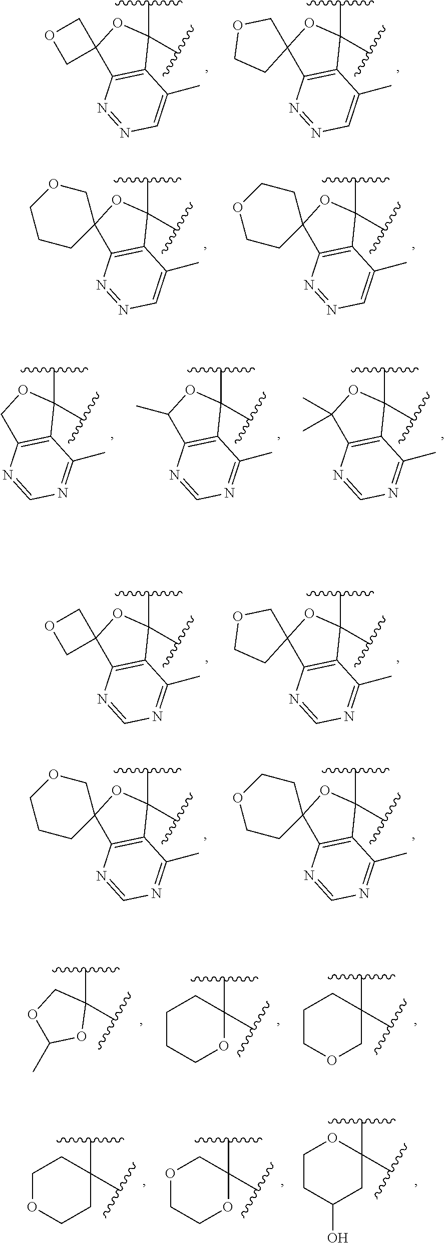

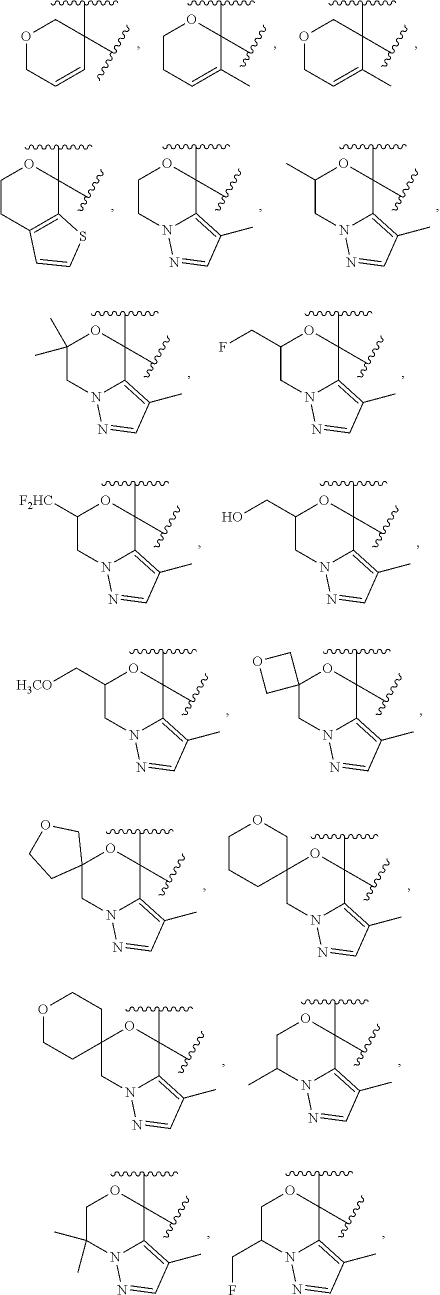

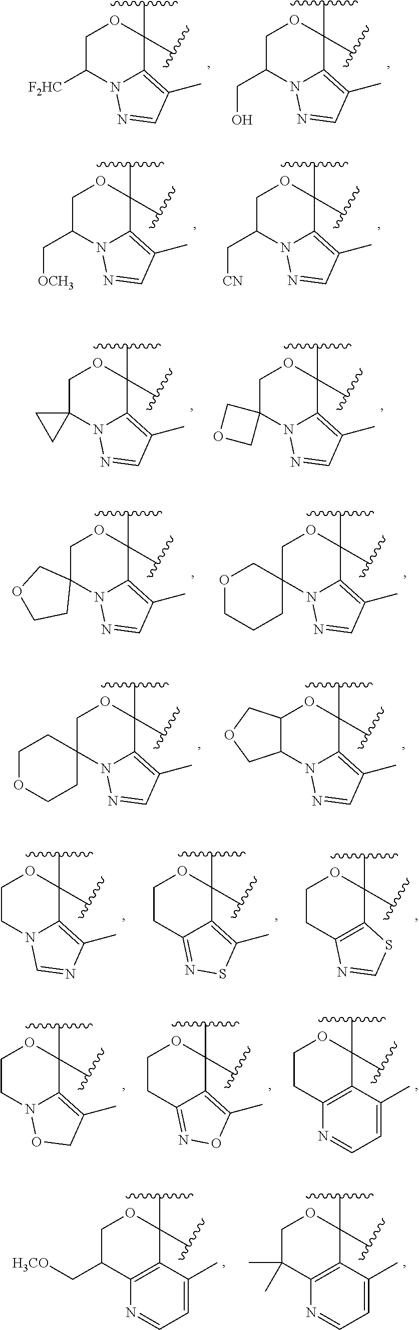

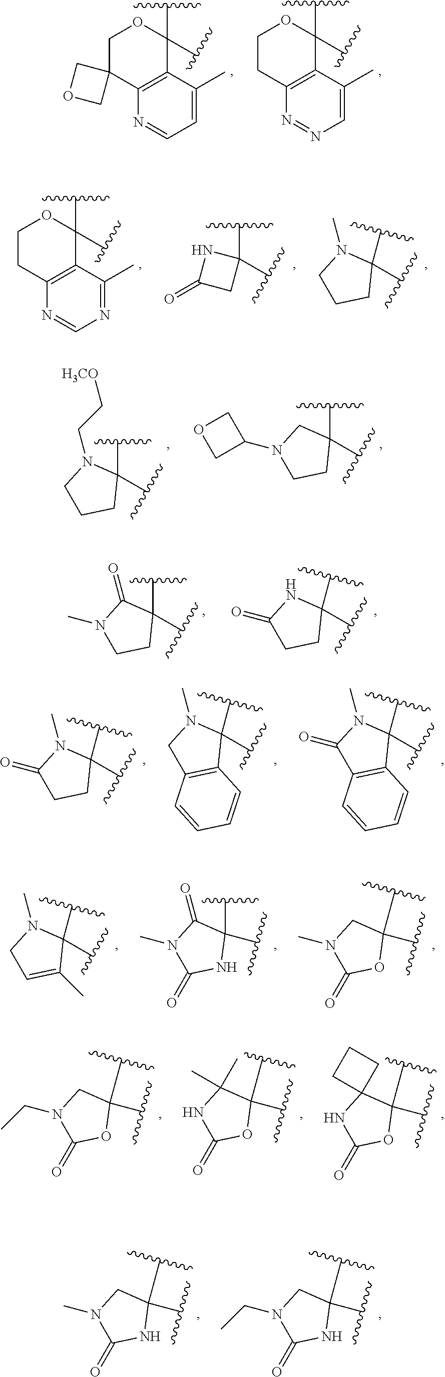

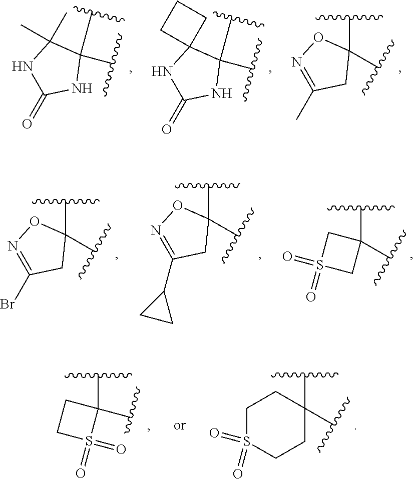

In some cases,

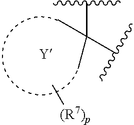

is

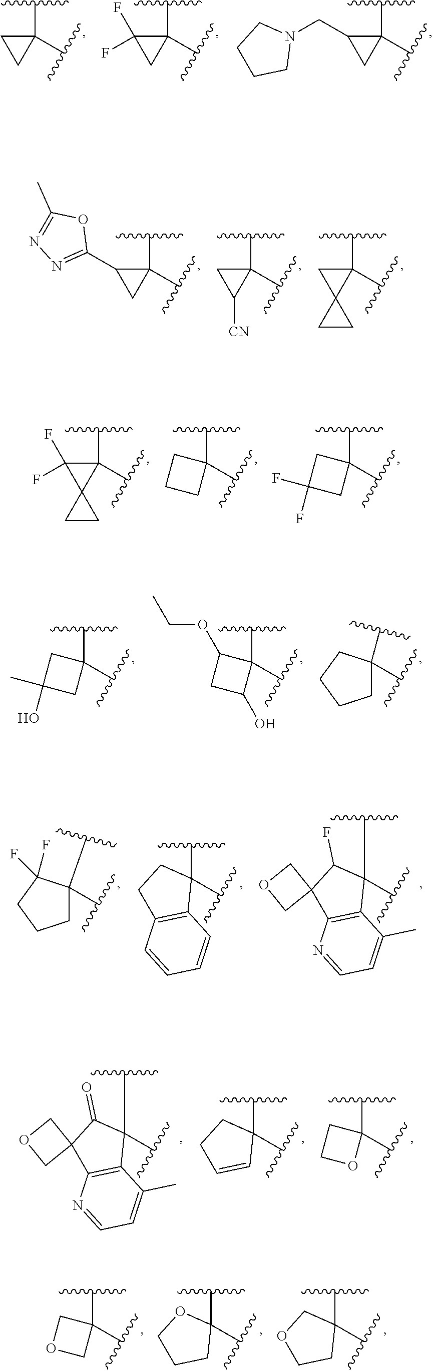

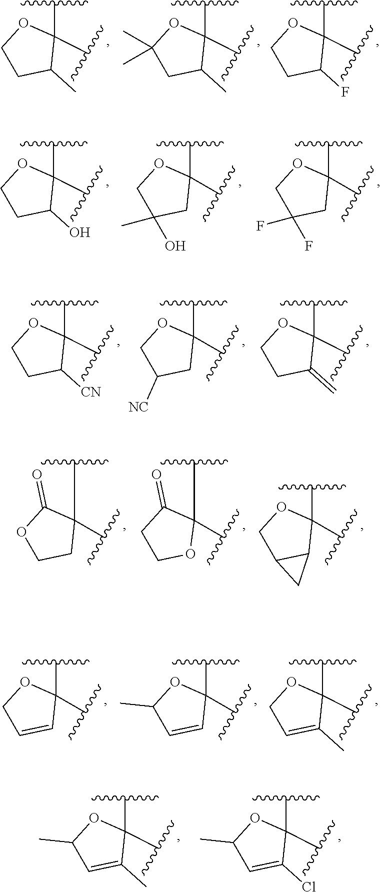

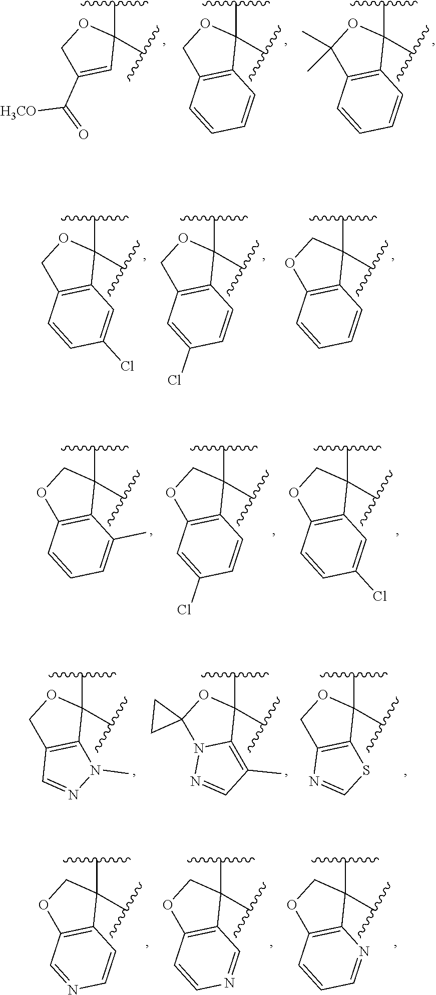

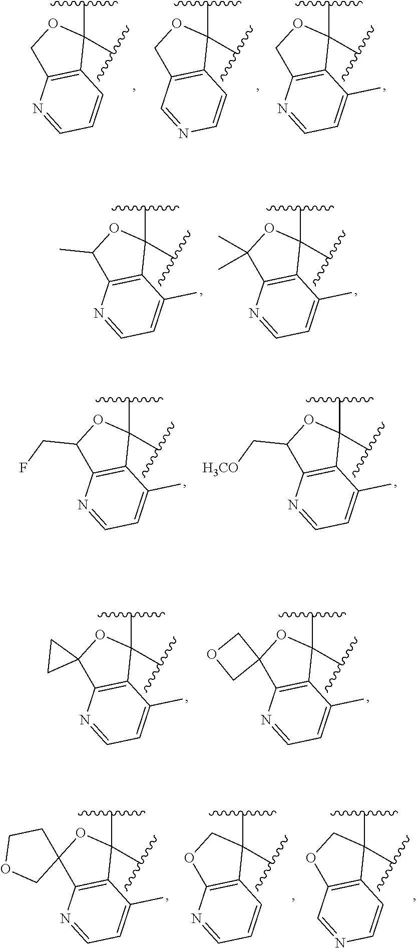

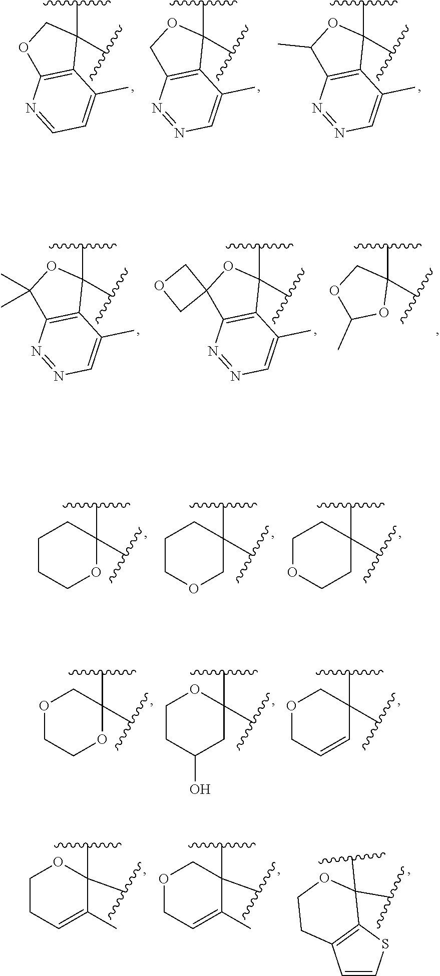

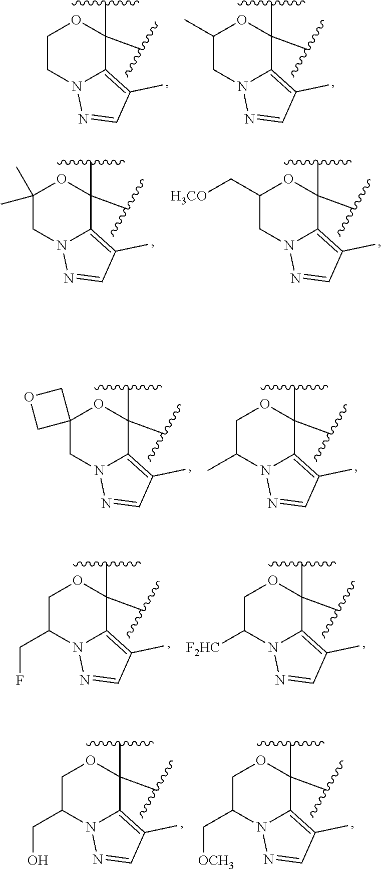

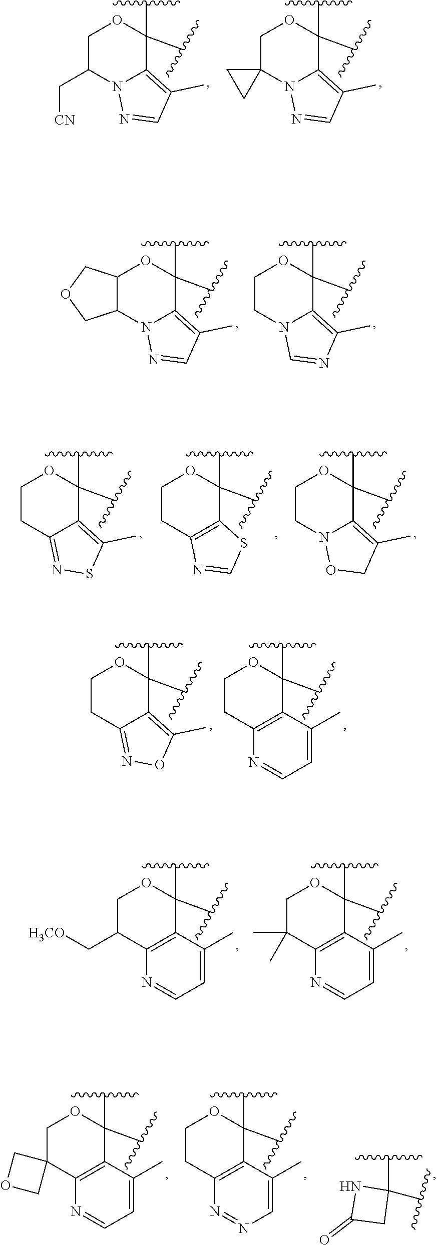

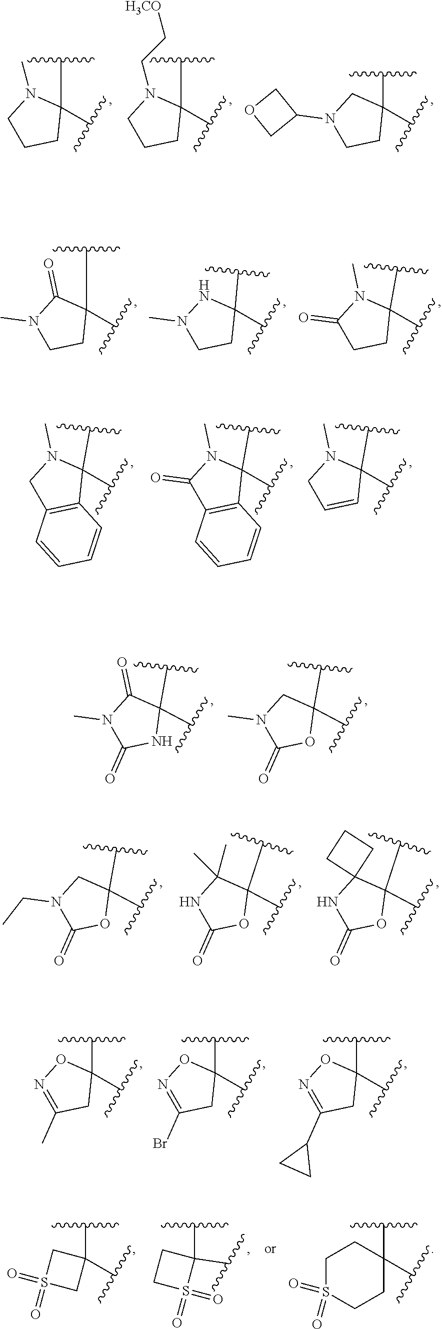

In some cases,

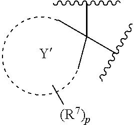

is

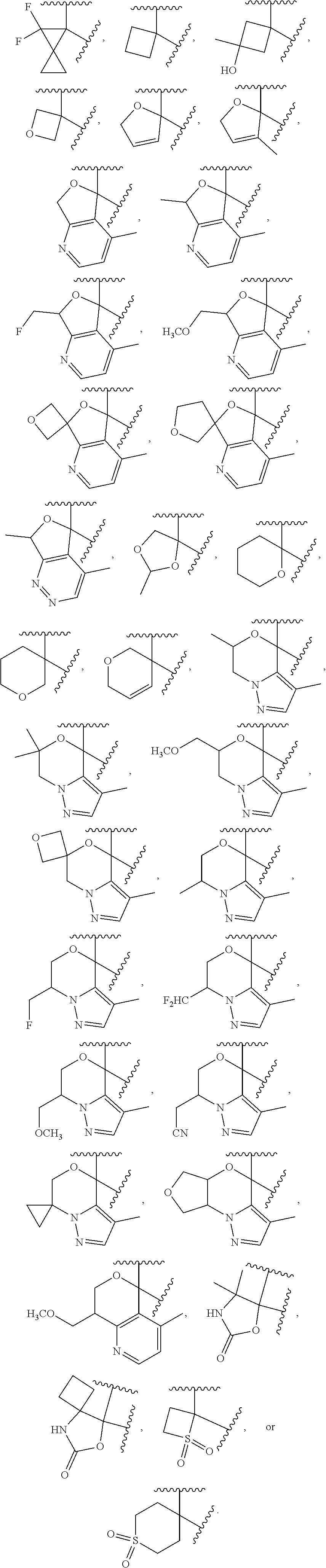

In some cases,

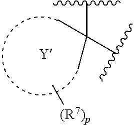

is

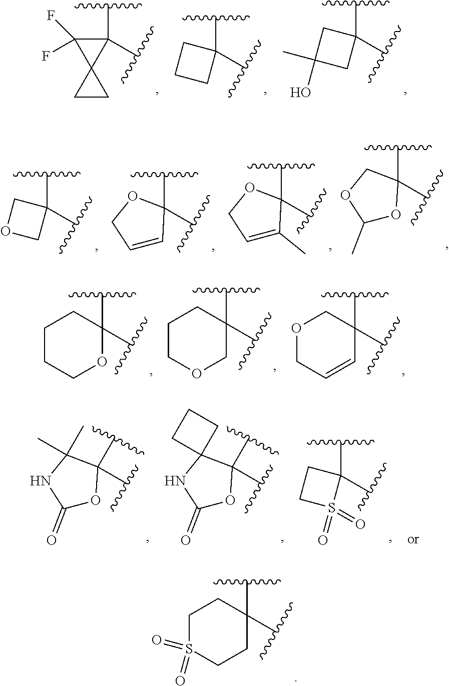

In some cases,

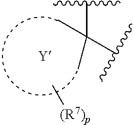

is

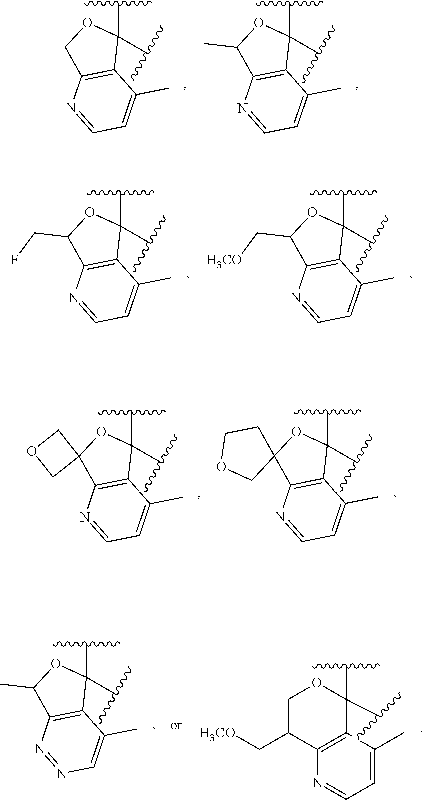

In some cases,

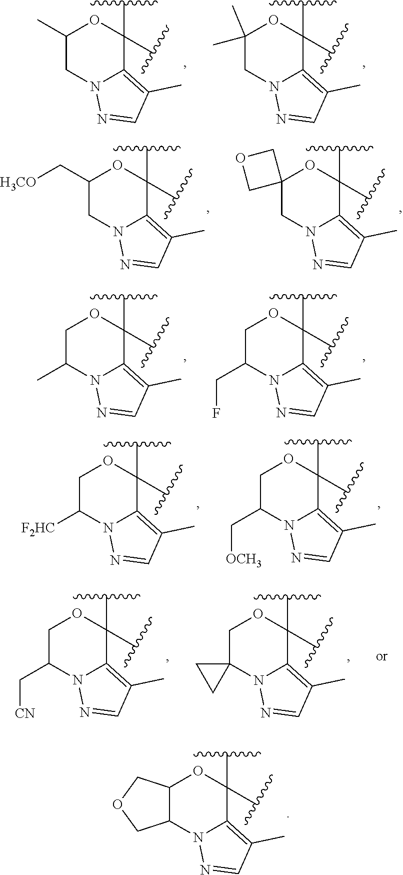

In some cases,

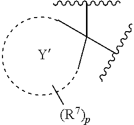

is

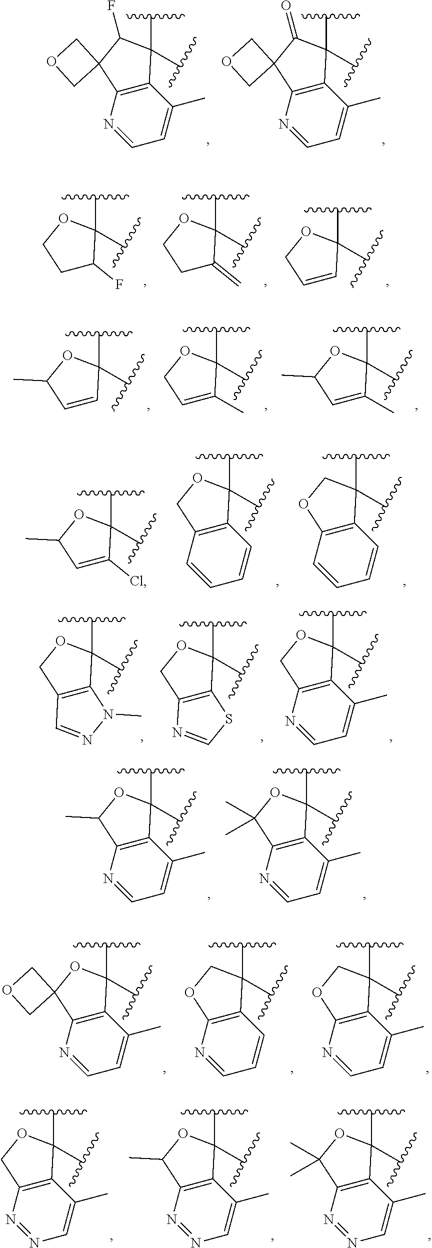

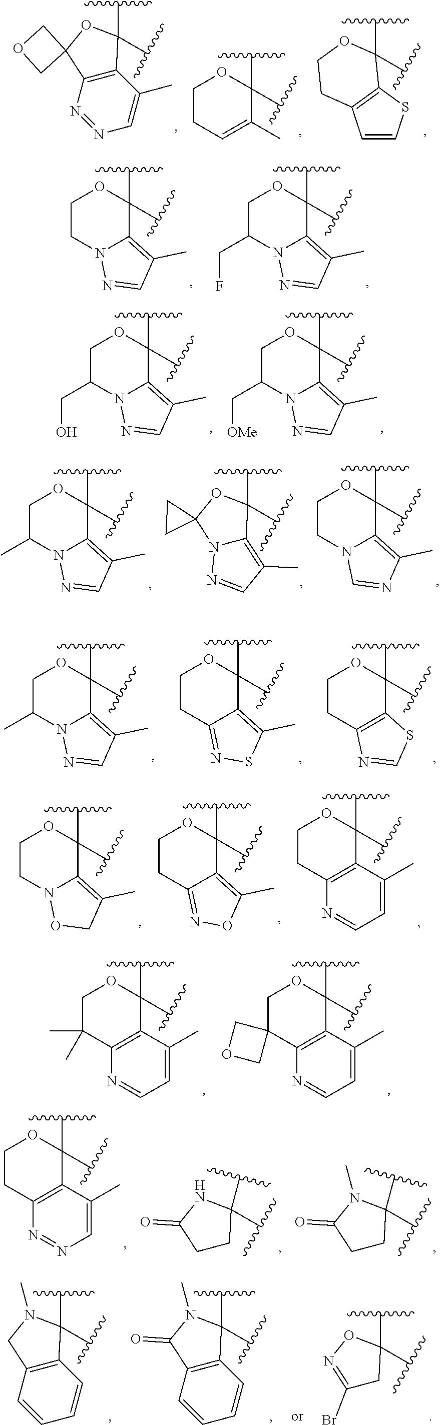

In some cases,

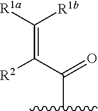

is

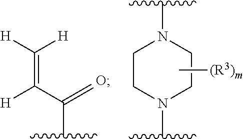

is

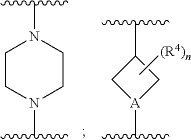

is

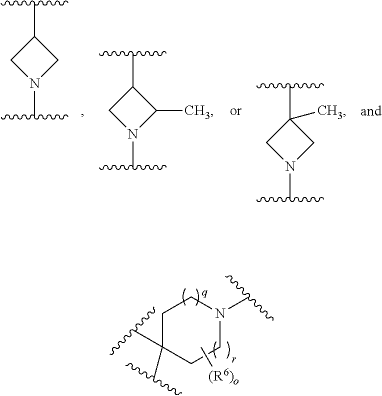

is

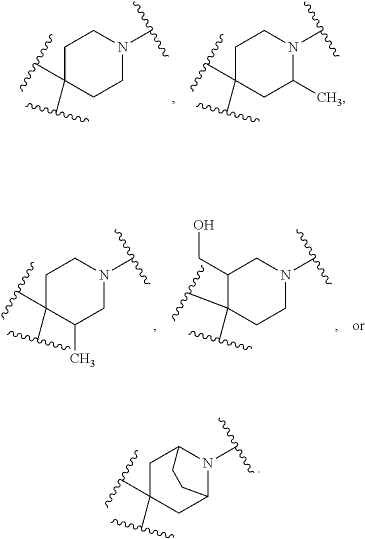

In some cases,

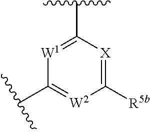

is

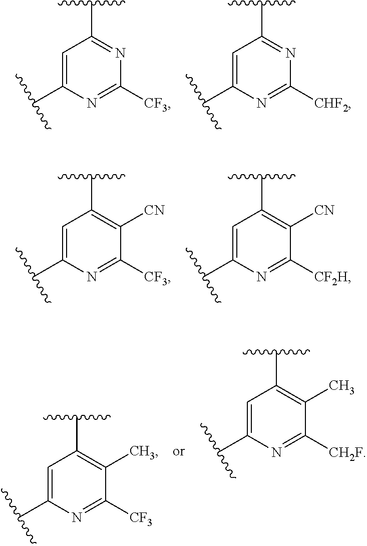

In some cases,

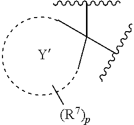

is

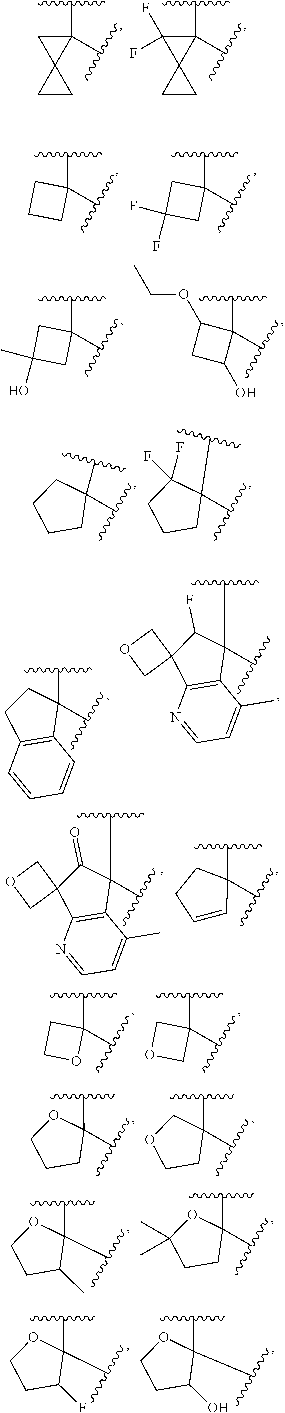

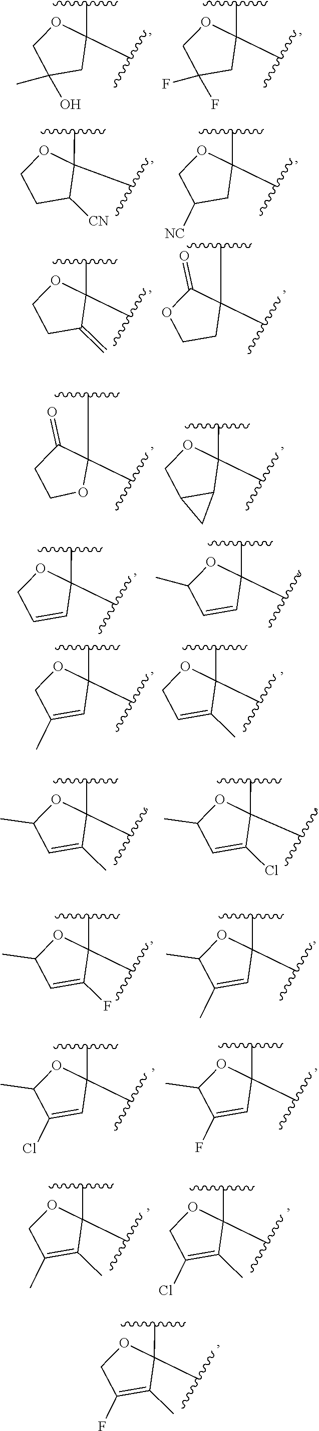

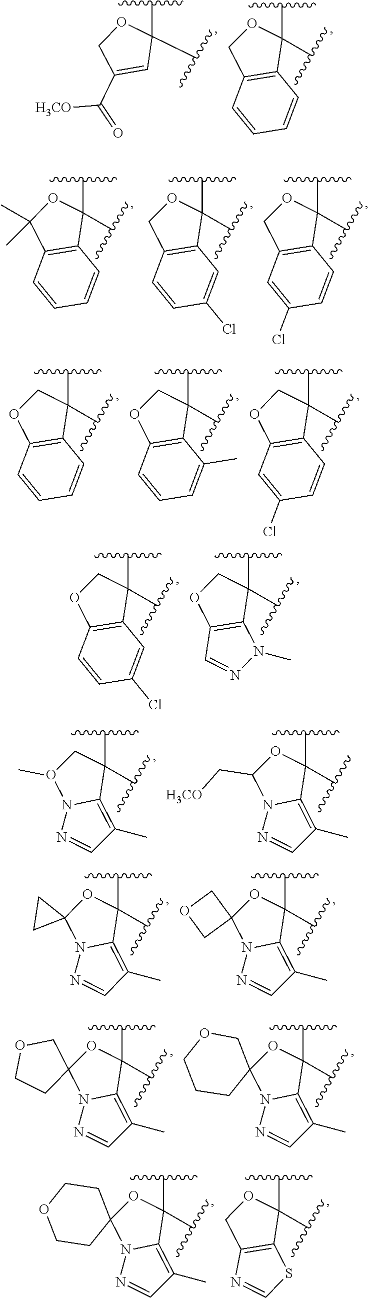

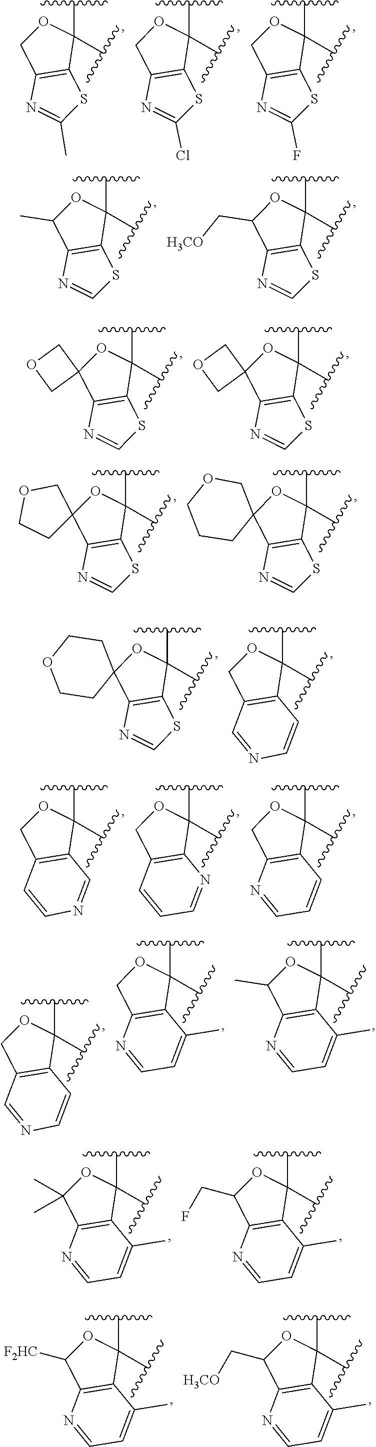

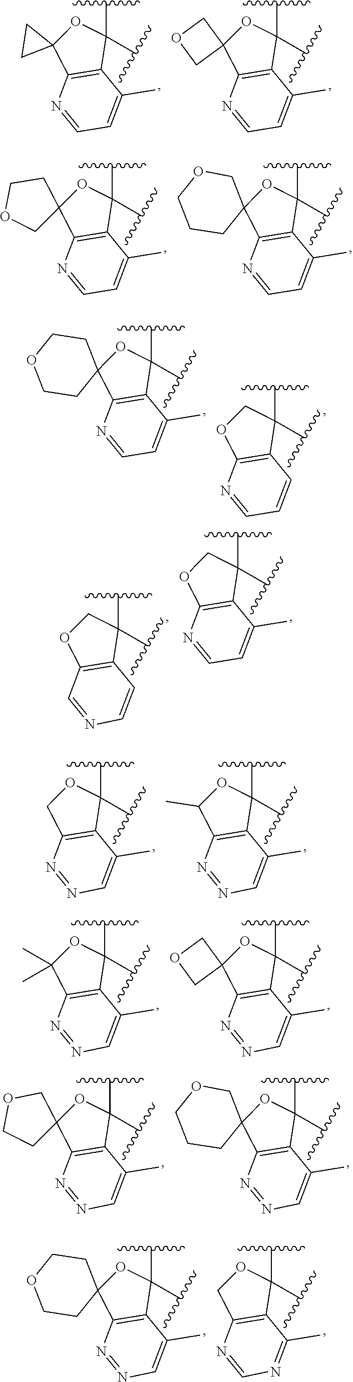

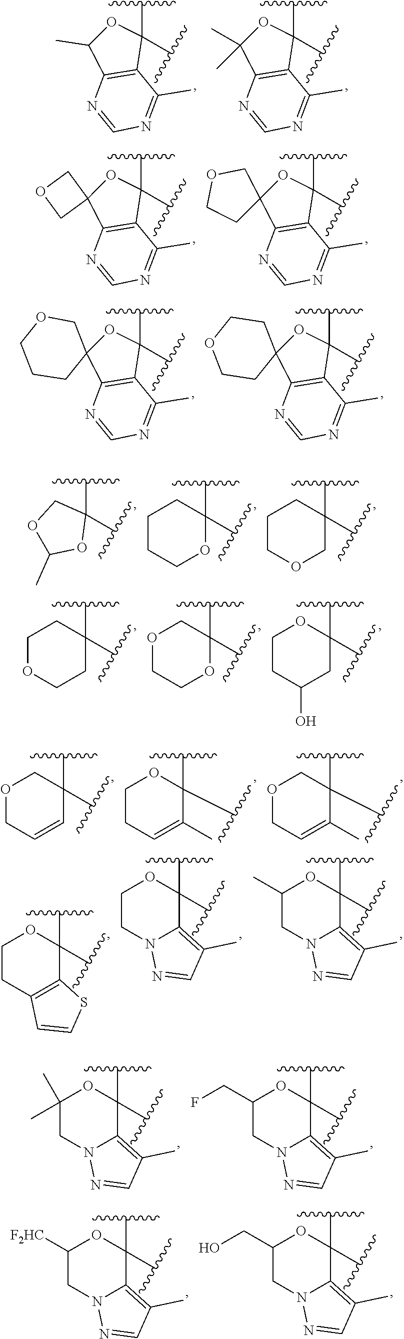

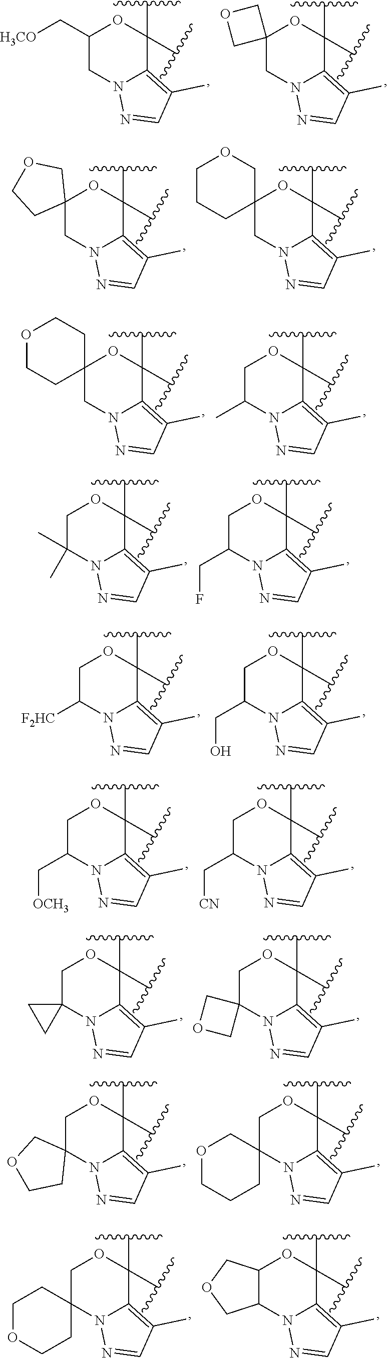

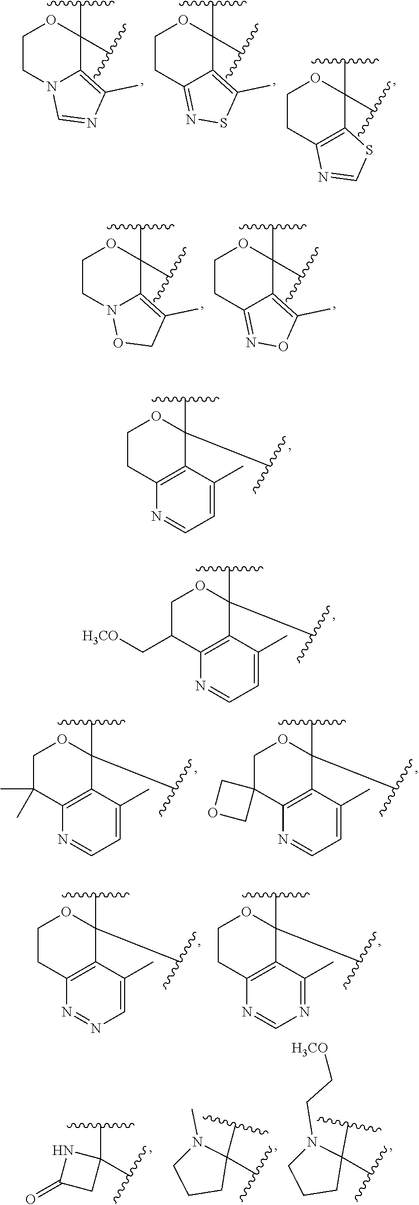

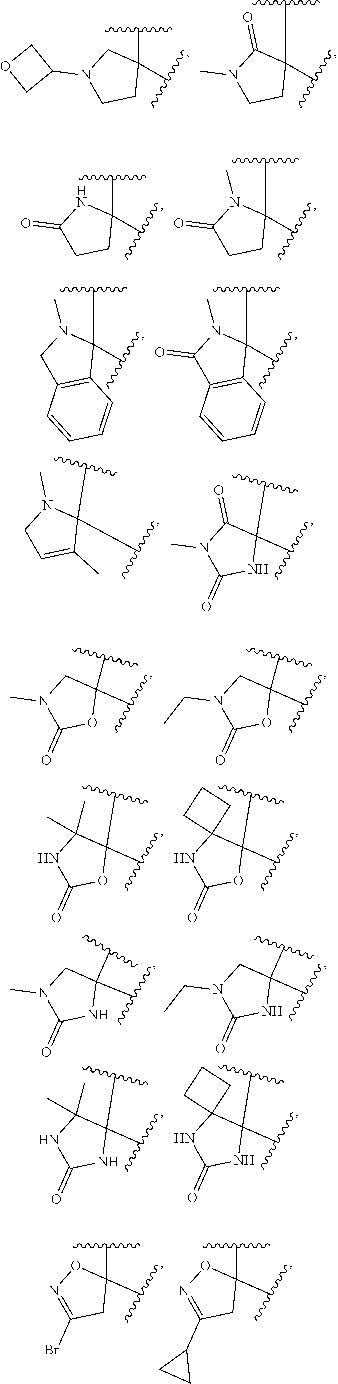

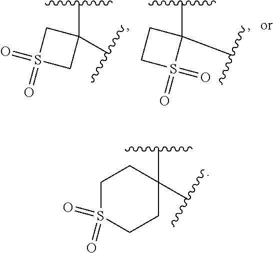

In some cases,

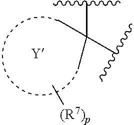

is

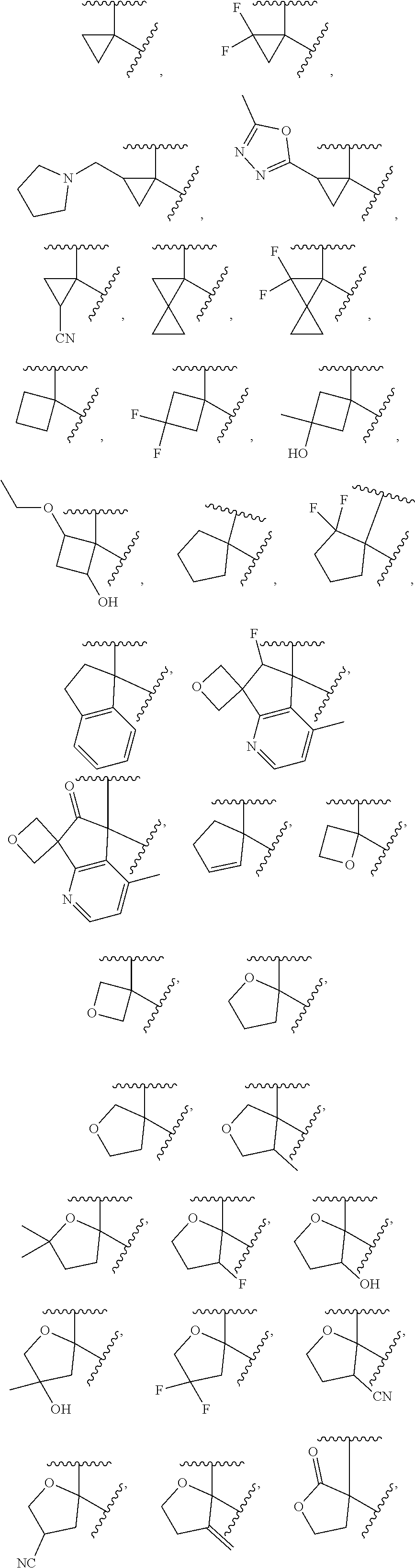

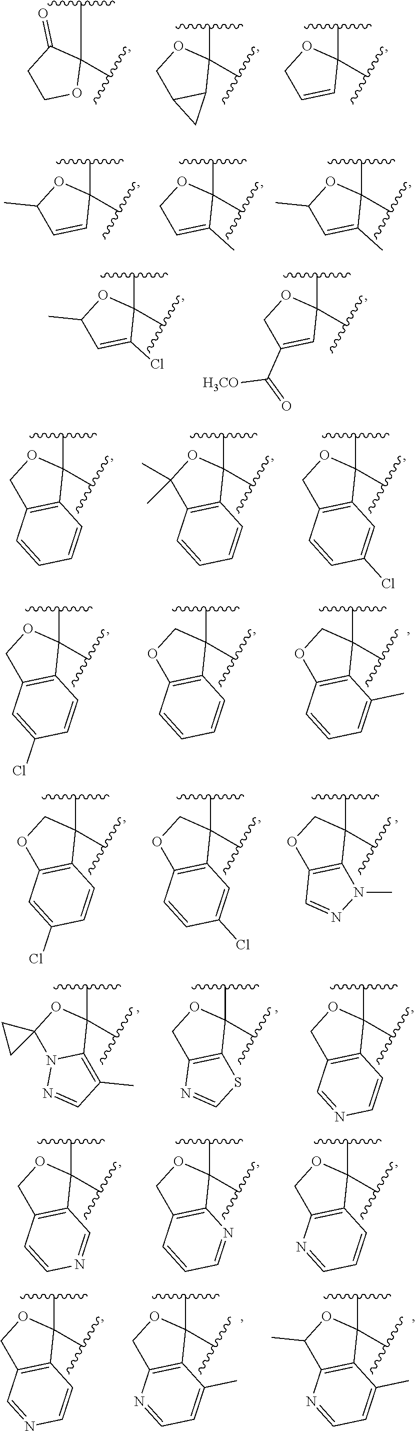

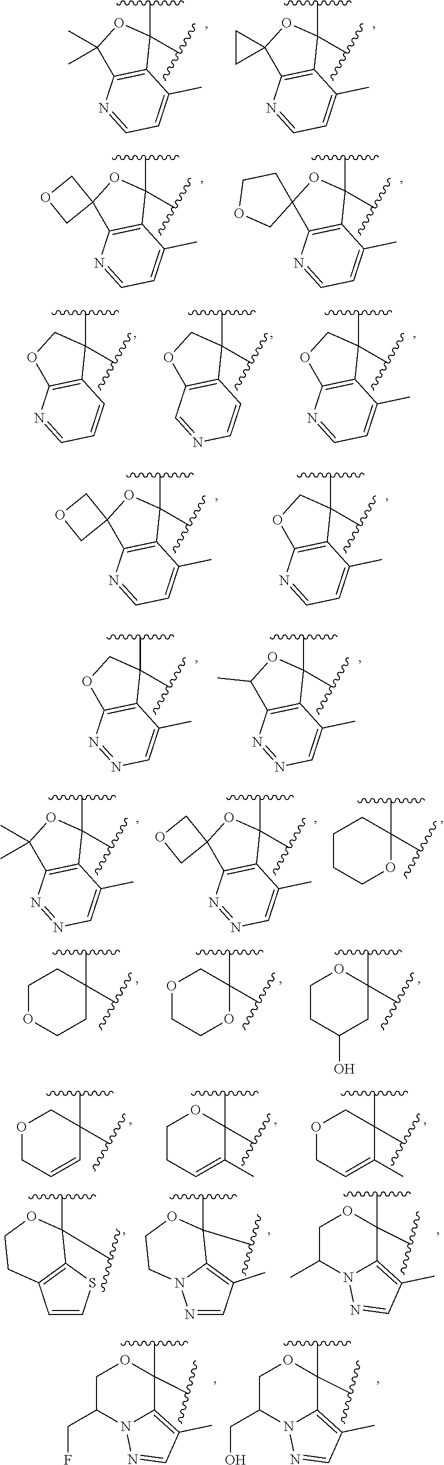

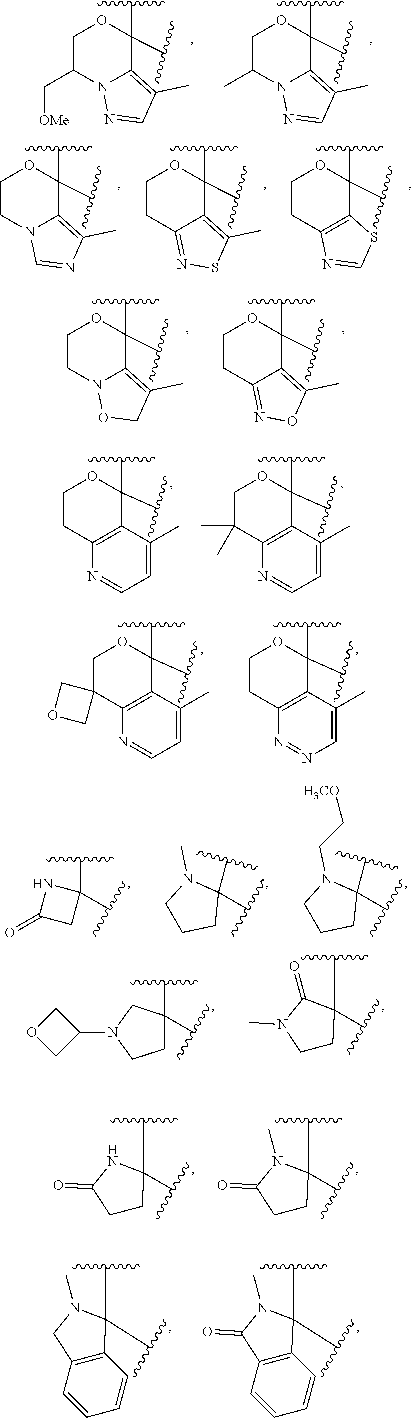

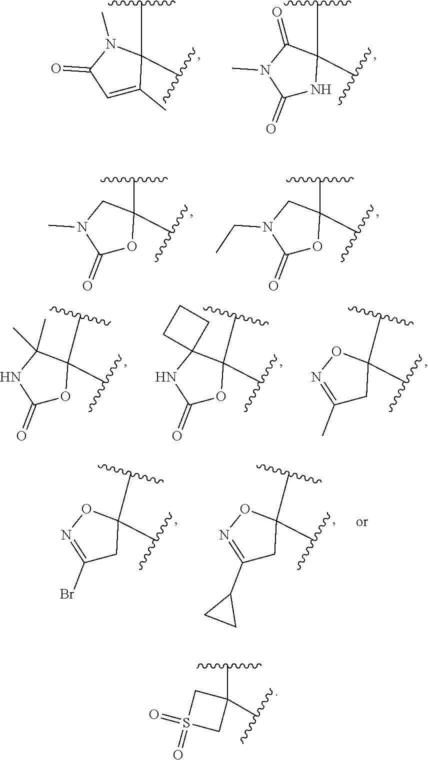

In some cases,

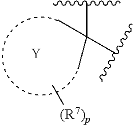

is

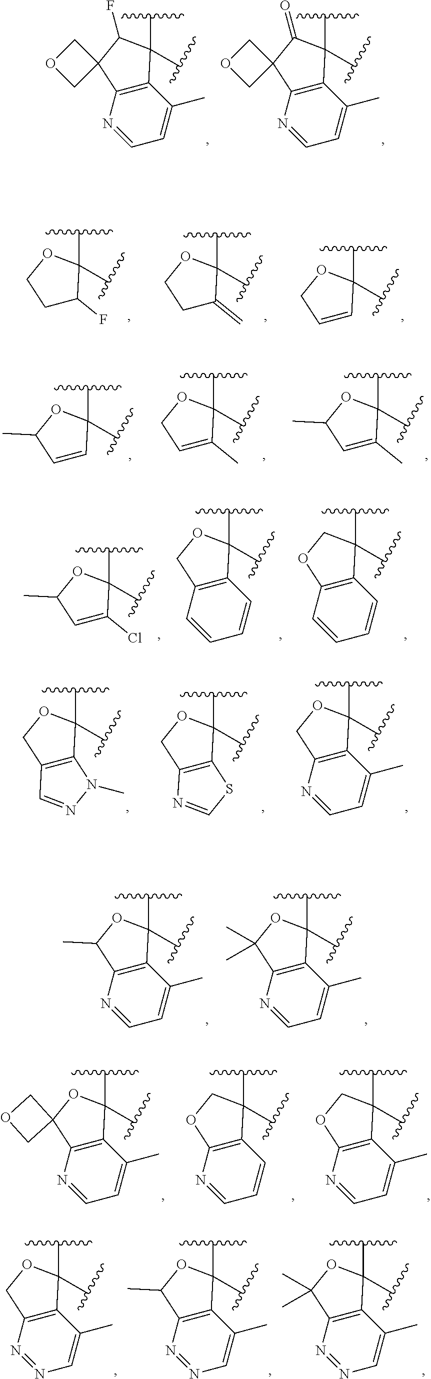

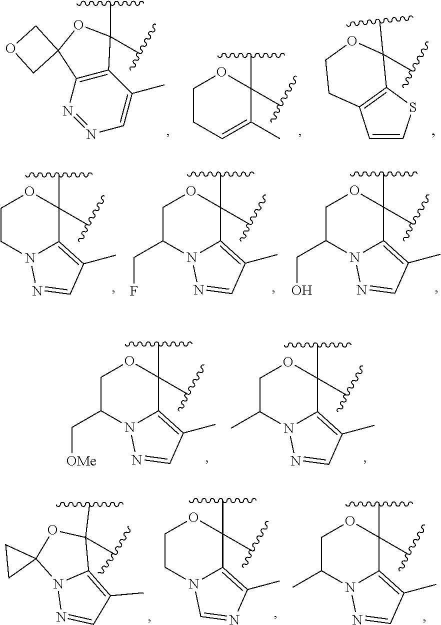

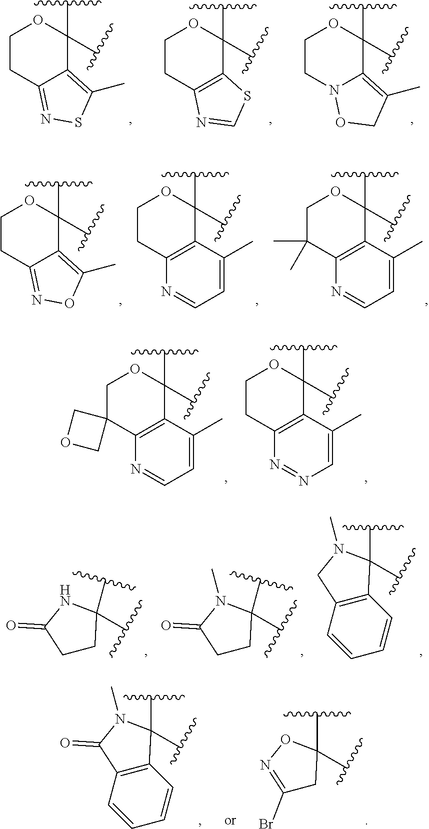

In some cases,

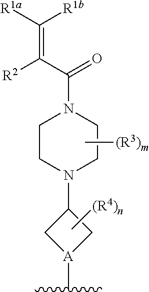

### of Formula (II) is

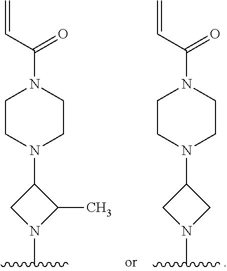 [SMILES: *N1CC(N2CCN(C(=O)C=C)CC2)C1C ; *N1CC(N2CCN(C(=O)C=C)CC2)C1]

In some cases,

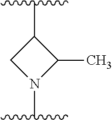 [SMILES: *C1CN(*)C1C]

exhibits the following stereochemical configuration:

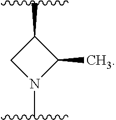 [SMILES: *[C@@H]1CN(*)[C@@H]1C]

In some cases,

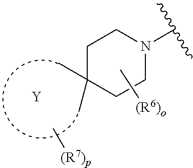

is

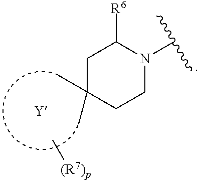

In some cases,

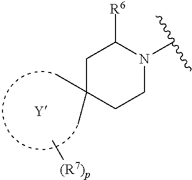

exhibits the following stereochemical configuration:

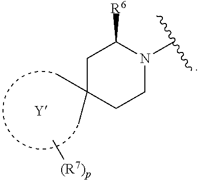

In some cases, R⁶ is CH₃.

It is understood that selections of values of each variable are those that result in the formation of stable or chemically feasible compounds.

### Compounds of Formula (I)

Further provided herein are compounds of Formula (I):

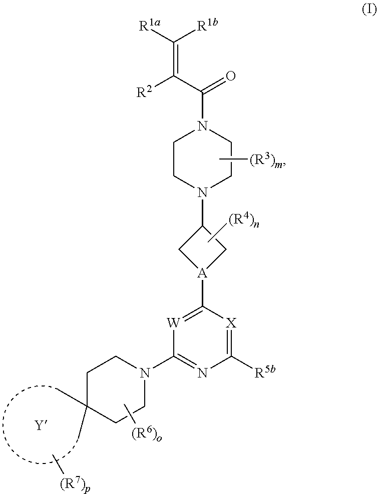

and pharmaceutically acceptable salts thereof, wherein:
m is 0, 1, 2, 3, or 4;
n is 0, 1, or 2;
is 0, 1, 2, 3, or 4;
p is 0, 1, 2, 3, 4, or 5;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
W is CH, C-halo, C—CN, C—C₁₋₃alkyl, C—C₁₋₃haloalkyl, C—C₁₋₃alkyleneOH, or C—C₀₋₃alkylene-C₁₋₄alkoxy;
X is N or C—R^{5a},
Y′ is cycloalkyl having 3 to 7 total ring atoms, cycloalkenyl having 4 to 7 total ring atoms, heterocycloalkyl having 3 to 7 total ring atoms and 1, 2, or 3 heteroatoms selected from N, O, and S, or heterocycloalkenyl having 5 to 7 total ring atoms and 1, 2, or 3 heteroatoms selected from N, O, and S;
each of R^{1a}, R^{1b}, and R² independently is H, D, halo, C₁₋₄alkyl, C₁₋₄ haloalkyl, C₁₋₂alkylene-OH, C₀₋₂alkylene-C₁₋₄alkoxy, C₀₋₂alkylene-C₁₋₆haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R² together with the carbon atoms to which they are attached from a

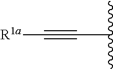

group;
each R³ independently is C₁₋₃alkyl, C₁₋₃haloalkyl,

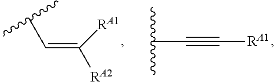

C₀₋₃alkyleneCN, C₀₋₃alkyleneOH, or C₀₋₃alkylene-C₁₋₃alkoxy; two geminal R³ form an oxo group; two geminal R³ together with the atom to which they are attached form a spiro-cycloalkyl ring having 3-7 total ring atoms or a spiro-heterocycloalkyl ring having 3-7 total ring atoms and 1 or 2 heteroatoms selected from N, O and S; or two adjacent R³ together with the atoms to which they are attached form a fused cycloalkyl ring having 3-7 total ring atoms;
each R⁴ independently is C₁₋₃alkyl, C₁₋₃haloalkyl, C₀₋₃alkyleneCN, C₁₋₃alkyleneOH, or C₁₋₃alkylene-C₁₋₃alkoxy; two geminal R³ form an oxo group; or two geminal R⁴ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S;
R^{5a} is H, CN, halo, C₁₋₃alkyl, C₁₋₃haloalkyl, C₂₋₆alkenyl, C₂₋₆alkynyl, C₀₋₃alkyleneOH, C₀₋₃alkylene-C₁₋₃alkoxy, or cycloalkyl having 3-5 total ring atoms;
R^{5b} is C₁₋₄alkyl, C₂₋₃alkenyl, C₂₋₃alkynyl, C₁₋₃haloalkyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N. O, and S, or heterocycloalkenyl having 5-7 total ring atoms and 1-3 heteroatoms selected from N, O, and S, wherein each of the alkyl, alkenyl, and alkynyl groups independently is unsubstituted or substituted with 1 or more substituents, or R^{5a} and R^{5b} together with the atoms to which they are attached form a cycloalkyl ring having 3-7 total ring atoms;
each R⁶ independently is halo, CN, C₁₋₃alkyl, C₁₋₃haloalkyl, C₀₋₃alkyleneOH, C₀₋₃alky lene-C₁₋₃alkoxy, deuterated C₀₋₃alkylene-C₁₋₃alkoxy, or C₁₋₄alkylene-N(R^{N1})₂; two geminal R⁶ form an oxo group; two geminal R⁶ together with the atom to which they are attached from a spiro-cycloalkyl ring having 3-7 total ring atoms or a spiro-heterocycloalkyl ring having 4-7 total ring atoms and 1 or 2 heteroatoms selected from N, O and S; or two adjacent R⁶ together with the atoms to which they are attached form a fused cycloalkyl ring having 3-7 total ring atoms; wherein the cycloalkyl or heterocycloalkyl ring of any of the foregoing is unsubstituted or substituted with 1 or more substituents; or two non-adjacent Rejoin together to form a C₁₋₃alkylene bridge or a C₁₋₃ether bridge;
each R⁷ independently is halo, CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, C₀₋₄alkylene-cycloalkyl having 3-7 total ring atoms, C₀₋₄alkylene-heterocycloalkyl having 3-7 total ring atoms and 1, 2, or 3 heteroatoms selected from N, O, and S, C₀₋₄alkylene-aryl having 6-10 total ring atoms, C₀₋₄alkylene-heteroaryl having 5-7 total ring atoms and 1, 2, or 3 heteroatoms selected from N, O, and S; two geminal R⁷ form CH₂ or an oxo group; two geminal R⁷ together with the atom to which they are attached from a spiro-cycloalkyl ring having 3-7 total ring atoms or a spiro-heterocycloalkyl ring having 4-7 total ring atoms and 1 or 2 heteroatoms selected from N, O and S; or two adjacent R⁷ together with the atoms to which they are attached form a fused cycloalkyl ring having 3-7 total ring atoms, a fused heterocycloalkyl ring having 3-7 total ring atoms and 1, 2, or 3 heteroatoms selected from N, O and S, a fused aryl ring having 6 total ring atoms, or a fused heteroaryl ring having 5-7 total ring atoms and 1, 2, or 3 heteroatoms selected from N, O, and S; wherein the cycloalkyl, heterocycloalkyl, aryl, or heteroaryl ring of any of the foregoing is unsubstituted or substituted with 1 or more substituents;
each of R^{A1} and R^{A2} independently is H, C₁₋₃alkyl, C₁₋₃haloalkyl, or cycloalkyl having 3-5 total ring atoms; and
each R^{N1} independently is H or C₁₋₄alkyl.

In some cases, the compound of Formula (I) is a free base. In some cases, the compound of Formula (I) is a pharmaceutically acceptable salt.

In some cases, R^{1a} is H or D. In some cases, R^{1a} is H. In some cases, R^{1a} is D. In some cases, R^{1b} is H or D. In some cases, R^{1b} is H. In some cases, R^{1b} is D. In some cases, R² is H or D. In some cases, R² is H. In some cases. R² is D. In some cases, at least one of R^{1a}, R¹², and R² is H or D. In some cases, at least one of R^{1a}, R^{1b}, and R² is H. In some cases, at least one of R^{1a}, R^{1b}, and R² is D. In some cases, at least two of R^{1a}, R^{1b}, and R² are each independently H or D. In some cases, at least two of R^{1a}, R^{1b}, and R² are H. In some cases, at least two of R^{1a}, R^{1b}, and R² are D. In some cases, each of R^{1a}, R^{1b}, and R² independently is H or D. In some cases, each of R^{1a}, R^{1b}, and R² independently is H. In some cases, each of R^{1a}, R^{1b}, and R² independently is D. In some cases, at least one of R^{1a}, R^{1b}, and R² is halo (e.g., Br, Cl, or F). In some cases, one of R^{1a}, R^{1b}, and R² is halo. In some cases, R^{1a} is halo and each of R^{1b} and R² is H. In some cases, at least one of R^{1a}, R^{1b}, and R² is Br, Cl, or F. In some cases, one of R^{1a}, R^{1b}, and R² is Br, Cl, or F. In some cases, R^{1a} is Br, Cl, or F and each of R^{1b} and R² is H. In some cases, at least one of R^{1a}, R^{1b}, and R² is Br or Cl. In some cases, one of R^{1a}, R^{1b}, and R² is Br or Cl. In some cases, R^{1a} is Br or Cl and each of R^{1b} and R² is H. In some cases, at least one of R^{1a}, R^{1b}, and R² is C₁₋₄alkyl or C₁₋₄haloalkyl. In some cases, one of R^{1a}, R^{1b}, and R² is C₁₋₄alkyl or C₁₋₄haloalkyl. In some cases, at least one of R^{1a}, R^{1b}, and R² is CH₃, CH₂CH₃, CH₂CH₂CH₃, CH(CH₃)₂, CH₂CH₂CH₂CH₃, CH₂F, CHF₂, or CF₃. In some cases, one of R^{1a}, R^{1b}, and R² is CH₃, CH₂CH₃, CH₂CH₂CH₃, CH(CH₃)₂, CH—CH₂CH₂CH₃, CH₂F, CHF₂, or CF₃. In some cases, at least one of R^{1a}, R^{1b}, and R² is CH₃, CH₂F, CHF₂, or CF₃. In some cases, one of R^{1a}, R^{1b}, and R² is CH₃, CH₂F, CHF₂, or CF₃. In some cases, at least one of R^{1a}, R^{1b}, and R² is C₁₋₂alkylene-OH, C₀₋₂alkylene-C₁₋₄alkoxy, C₀₋₂alkylene-C₁₋₄haloalkoxy, C₀₋₃alkylene-CN, or C₀₋₂alkylene-N(R^{N1})₂, and each R^{N1} independently is H or C₁₋₄alkyl. In some cases, each R^{N1} independently is H or CH₃. In some cases, each R^{N1} independently is H. In some cases, at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, at least one of R^{1a}, R^{1b}, and R² is C₁₋₂alkylene-heterocycloalkyl wherein the heterocycloalkyl group contains 3-6 total ring atoms and 1 or 2 heteroatoms selected from N, O, and S. In some cases, the heterocycloalkyl is aziridinyl, oxiranyl, azetidinyl, oxetanyl, pyrrolidinyl, tetrabydrofuranyl, imidazolidinyl, pyrazolidinyl, oxathiolidinyl, isoxthiodinyl, oxazolidinyl, isoxazolidinyl, thiazolidinyl, isothiazolidinyl, piperidinyl, diazinyl, or morpholinyl. In some cases, the heterocycloalkyl is aziridinyl, azetidinyl, pyrrolidinyl, piperidinyl, or morpholinyl. In some cases, at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, R^{1b} and R² together with the carbon atoms to which they are attached from

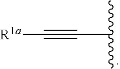

In some cases, R^{1a} is H. In some cases, R^{1b} and R² together with the carbon atoms to which they are attached from

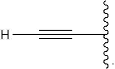 [SMILES: *C#C]

In some cases,

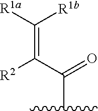

is

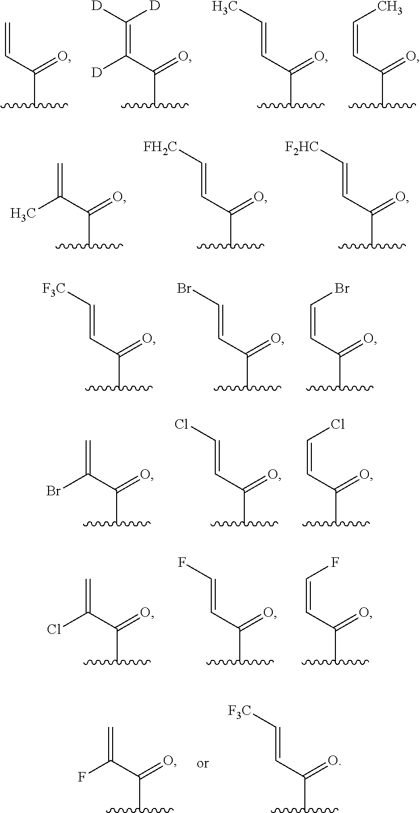

In some cases,

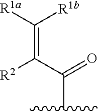

is

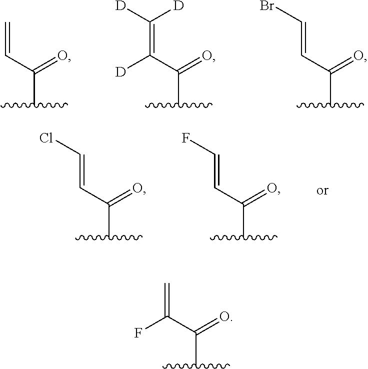

In some cases,

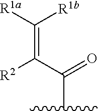

is

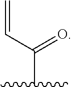 [SMILES: *C(=O)C=C]

In some cases, m is 0, and

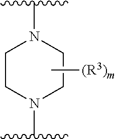

is

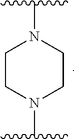 [SMILES: *N1CCN(*)CC1]

In some cases, m is 1. In some cases, m is 2. In some cases, m is 3. In some cases, m is 4. In some cases,

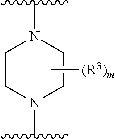

is deuterated. In some cases,

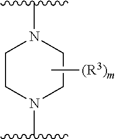

is fully deuterated. In some cases,

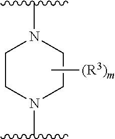

is

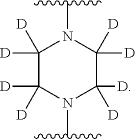

In some cases, at least one R³ is C₁₋₃alkyl or C₁₋₃haloalkyl. In some cases, at least one R³ is CH₃, CH₂CH₃, CH₂CH₂CH₃, CH(CH₃)₂, CF₃, CHF₂, or CH₂F. In some cases, at least one R³ is CH₃, CH₂CH₃, CF₃, CHF₂, or CH₂F. In some cases, m is 1 or 2 and each R³ is CH₃. In some cases, m is 1 and R³ is CF₃, CHF₂, or CH₂F. In some cases, at least one R³ is

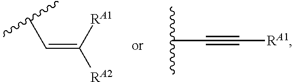

and each of R^{A1} and R^{A2} independently is H, C₁₋₃alkyl, C₁₋₃haloalkyl, or cycloalkyl having 3-5 total ring atoms. In some cases, m is 1 and R³ is

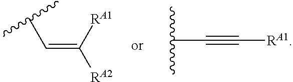

In some cases, each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl. In some cases,

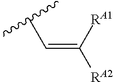

is

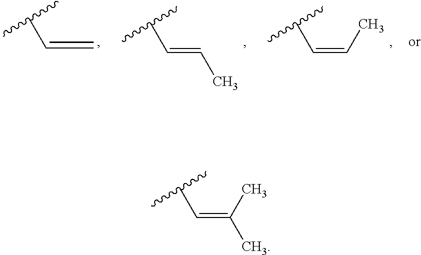

In some cases,

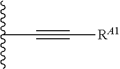

is

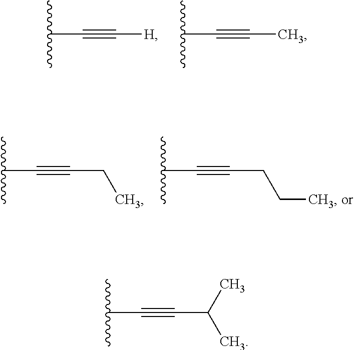

In some cases,

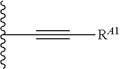

is

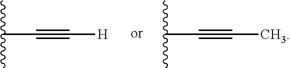

In some cases, at least one R³ is

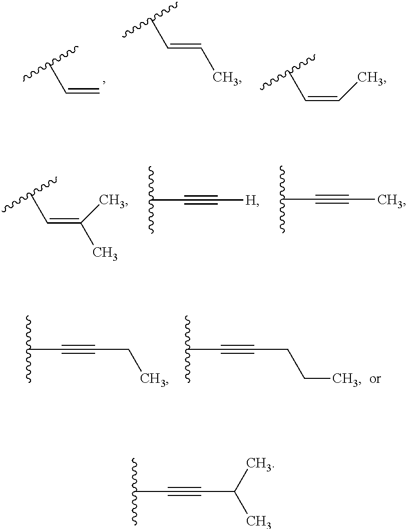

In some cases, at least one R³ is

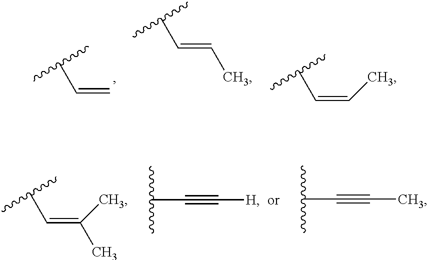

In some cases, at least one R³ is C₀₋₃alkyleneCN. In some cases, at least one R³ is CN, CH₂CN, or CH₂CH₂CN. In some cases, at least one R³ is CN or CH₂CN. In some cases, m is 1 and R³ is CN or CH₂CN. In some cases, at least one R³ is C₀₋₃alkyleneOH or C₀₋₃alkylene-C₁₋₃alkoxy. In some cases, at least one R³ is OH, CH₂OH, CH₂CH₂OH, OCH₃, CH₂OCH₃, or CH₂CH₂OCH₃. In some cases, m is 1 and R³ is OH, CH₂OH, CH₂CH₂OH, OCH₃, CH₂OCH₃, or CH₂CH₂OCH₃. In some cases, two geminal R³ form an oxo group (═O). In some cases, two geminal R³ together with the atom to which they are attached form a spiro-cycloalkyl ring having 3-7 total ring atoms or a spiro-heterocycloalkyl ring having 3-7 total ring atoms and 1 or 2 heteroatoms selected from N, O and S. In some cases, the spiro-cycloalkyl ring is spiro-cyclopropyl, spiro-cyclobutyl, or spiro-cyclopentyl. In some cases, the spiro-heterocycloalkyl ring is spiro-azetidinyl, spiro-oxetanyl, spiro-pyrrolidinyl, spiro-imidazolidinyl, spiro-pyrazolidinyl, or spiro-tetrahydrofuranyl. In some cases, two geminal R³ together with the atom to which they are attached form spiro-cyclopropyl, spiro-cyclobutyl, spiro-cyclopentyl, spiro-azetidinyl, spiro-oxetanyl, spiro-pyrrolidinyl, spiro-imidazolidinyl, spiro-pyrazolidinyl, or spiro-tetrahydrofuranyl. In some cases, two adjacent R³, together with the atoms to which they are attached, form a fused cycloalkyl ring having 3-7 total ring atoms. In some cases, two adjacent R³, together with the atoms to which they are attached, form a fused-cyclopropyl ring, a fused-cyclobutyl ring, a fused-cyclopentyl ring, or a fused-cyclohexyl ring. In some cases, two adjacent R³, together with the atoms to which they are attached, form a fused-cyclopropyl ring or a fused-cyclobutyl ring. In some cases, each R³ independently is CH₃, CH₂CH₃, CF₃, CHF₂, CH₂F,

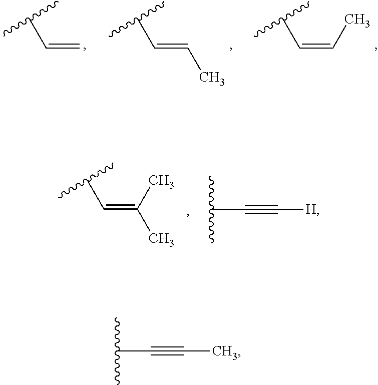

CN, CH₂CN, OH, CH₂OH, CH₂CH₂OH, OCH₃, CH₂OCH₃, or CH₂CH₂OCH₃; two geminal R³ form an oxo group (═O); two geminal R³ together with the atom to which they are attached form spiro-cyclopropyl, spiro-cyclobutyl, spiro-oxetanyl, or spiro-tetrahydrofuranyl group; or two adjacent R³ together with the atoms to which they are attached form a fused cyclopropyl ring or a fused cyclobutyl ring. In some cases, m is 1 and R³ is CH₃, CF₃, CHF₂, CH₂F, CN, CH₂CN, CH₂OH, CH₂OCH₃, or spiro-oxetanyl. In some cases,

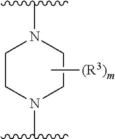

is

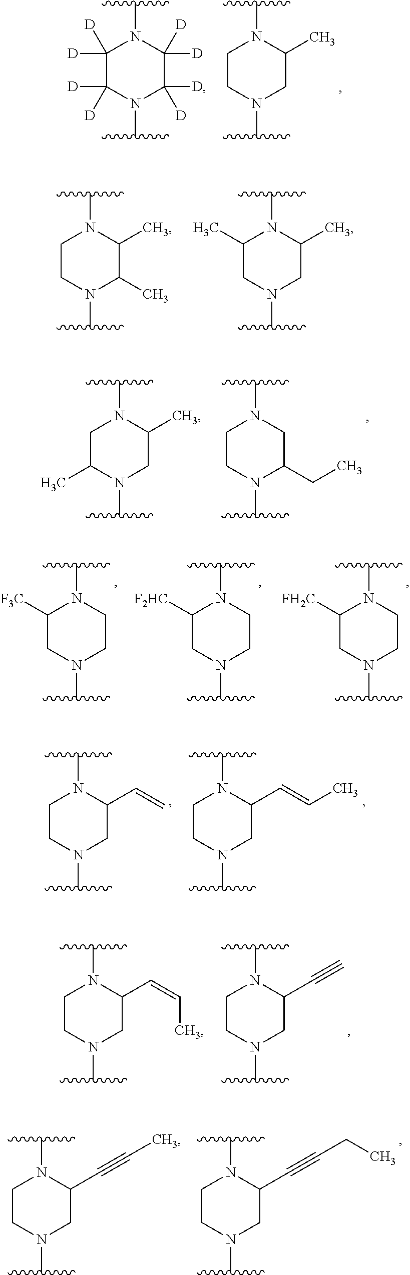

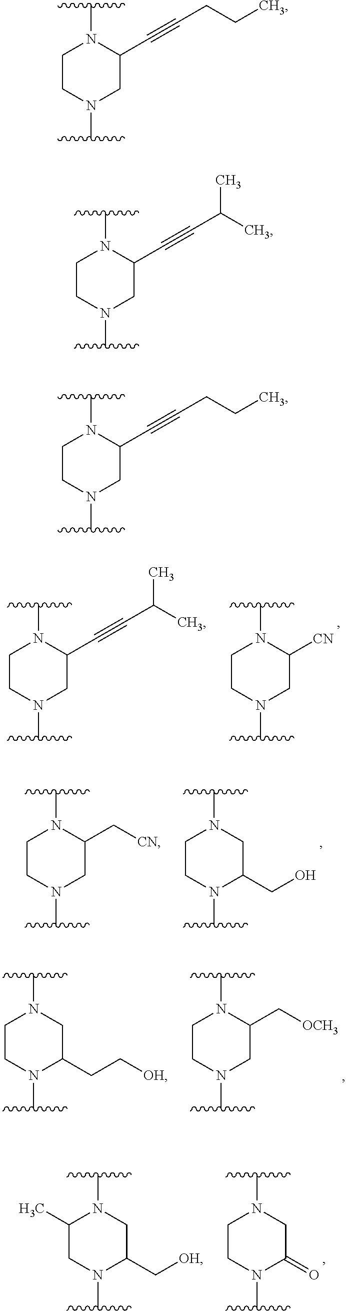

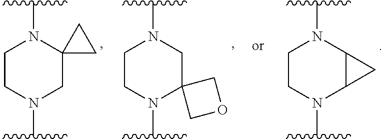

In some cases,

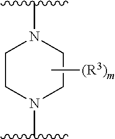

is

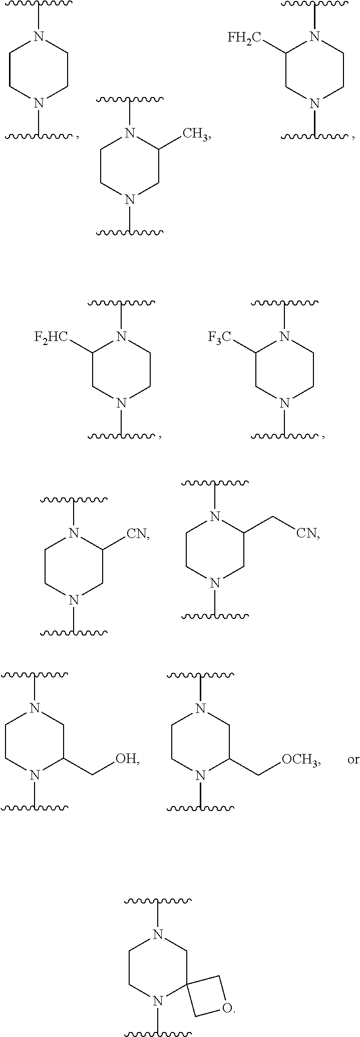

In some cases,

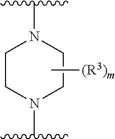

is

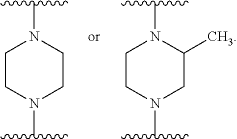 [SMILES: *N1CCN(*)CC1 ; *N1CCN(*)C(C)C1]

In some cases, A is N. In some cases, A is CH, C-halo, C—CN, C—C₁₋₃alkyl, C—C₁₋₃haloalkyl, C—C₀₋₃alkyleneOH, or C—C₀₋₃alkylene-C₁₋₄alkoxy. In some cases, A is CH. In some cases, A is C-halo or C—CN. In some cases, A is C—F or C—Cl. In some cases, A is C—F. In some cases, A is C—CN. In some cases, A is C—C₁₋₃alkyl or C—C₁₋₃haloalkyl. In some cases, A is C—CH₃, C—CH₂CH₃, C—CH₂CH₂CH₃, C—CH(CH₃)₂, C—CF₃, C—CHF₂, or C—CH₂F. In some cases, A is C—CH₃, C—CH₂F, C—CHF₂, or C—CF₃. In some cases. A is C—C₀₋₃alkyleneOH or C—C₀₋₃alkylene-C₁₋₄alkoxy. In some cases, A is C—OH, C—CH₂OH, C—CH₂CH₂OH, C—OCH₃, C—CH₂OCH₃, or C—CH₂CH₂OCH₃. In some cases, A is C—OH, C—CH₂OH, C—OCH₃, or C—CH₂OCH₃, In some cases, A is N, CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, A is N, CH, C—F, C—Cl, C—CN, C—CH₃, C—CF₃, C—CHF₂, C—CH₂F, C—OH, C—CH₂OH, C—OCH₃, or C—CH₂OCH₃. In some cases, n is 0. In some cases, n is 1. In some cases, n is 2. In some cases, at least one R⁴ is C₁₋₃alkyl or C₁₋₃haloalkyl. In some cases, at least one R⁴ is CH₃, CH₂CH₃, CH₂CH₂CH₃, CH(CH₃)₂, CF₃, CHF₂, or CH₂F. In some cases, one R⁴ is CH₃, CH₂CH₃, CH₂CH₂CH₃, CH(CH₃)₂, CF₃, CHF₂, or CH₂F. In some cases, n is 2 and each R⁴ independently is CH₃, CH₂CH₃, CH₂CH₂CH₃, CH(CH₃)₂, CF₃, CHF₂, or CH₂F. In some cases, n is 1 and R⁴ is CH₃, CH₂CH₃, CH₂CH₂CH₃, CH(CH₃)₂, CF₃, CHF₂, or CH₂F. In some cases, n is 1 and R⁴ is CH₃. In some cases, at least one R⁴ is C₀₋₃alkyleneCN. In some cases, at least one R⁴ is CN or CH₂CN. In some cases, n is 1 and R⁴ is CN or CH₂CN. In some cases, at least one R⁴ is C₁₋₃alkyleneOH or C₁₋₃alkylene-C₁₋₃alkoxy. In some cases, at least one R⁴ is CH₂OH. CH₂CH₂OH, OCH₃, CH₂OCH₃, or CH₂CH₂OCH₃. In some cases, n is 1 and R⁴ is CH₂OH, CH₂CH₂OH, OCH₃, CH₂OCH₃, or CH₂CH₂OCH₃. In some cases, two geminal R⁴ form an oxo group (═O)). In some cases, two geminal R⁴ together with the atom to which they are attached from a spiro-cycloalkyl ring having 3-7 total ring atoms. In some cases, the spiro-cycloalkyl ring is spiro-cyclopropyl, spiro-cyclobutyl, or spiro-cyclopentyl. In some cases, the spiro-cycloalkyl ring is spiro-cyclopropyl or spiro-cyclobutyl. In some cases, two geminal R⁴ together with the atom to which they are attached form a spiro-heterocycloalkyl ring having 4-7 total ring atoms and 1 or 2 heteroatoms selected from N, O and S. In some cases, the spiro-heterocycloalkyl ring is spiro-oxetanyl or spiro-tetrahydrofuranyl. In some cases, the spiro-heterocycloalkyl ring is spiro-oxetanyl. In some cases, two geminal R⁴ together with the atom to which they are attached form spiro-cyclopropyl, spiro-cyclobutyl, or spiro-oxetanyl. In some cases, each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, OCH₃, CH₂OCH₃, CH₂CH₂OCH₃; or two geminal R⁴ together with the atom to which they are attached from an oxo group, spiro-cyclopropyl, spiro-cyclobutyl, or spiro-oxetanyl. In some cases, each R⁴ independently is CH₃, CH₂CH₃, CH₂CH₂CH₃, CH₂F, CN, CH₂CN, CH₂OH, CH₂CH₂OH, CH₂OCH₃, or two geminal R⁴ together with the atom to which they are attached from spiro-cyclopropyl. In some cases,

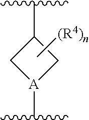

is

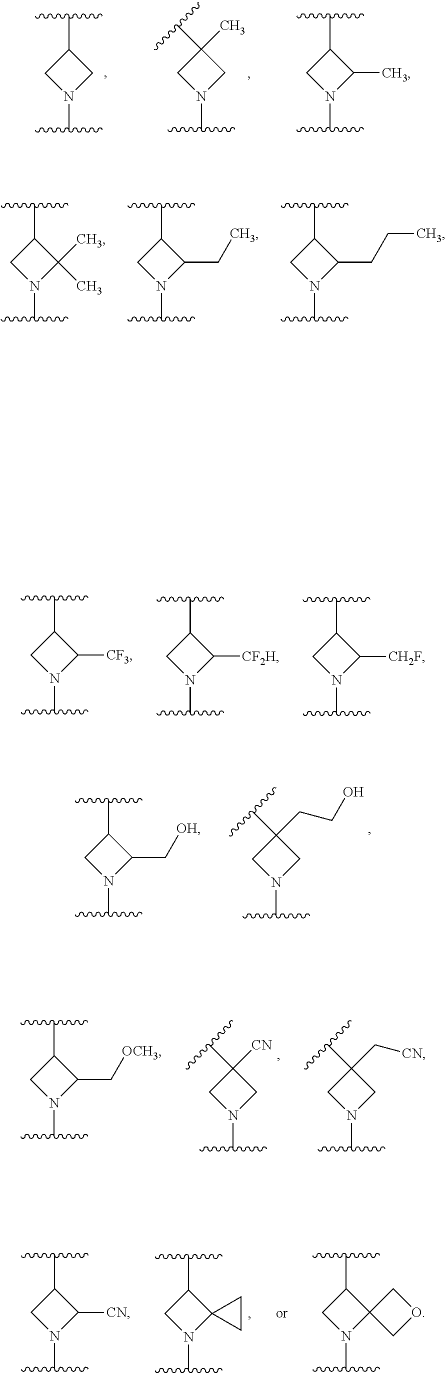

In some cases,

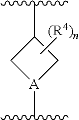

is

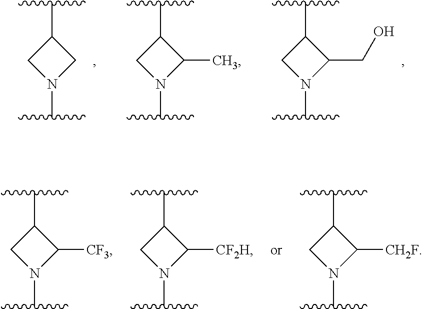

In some cases,

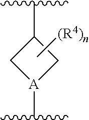

is

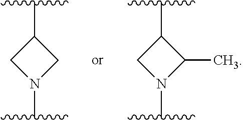 [SMILES: *C1CN(*)C1 ; *C1CN(*)C1C]

In some cases,

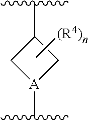

is

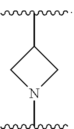 [SMILES: *C1CN(*)C1]

In some cases,

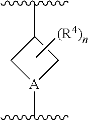

is

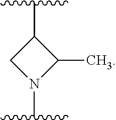 [SMILES: *C1CN(*)C1C]

In some cases,

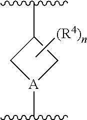

is

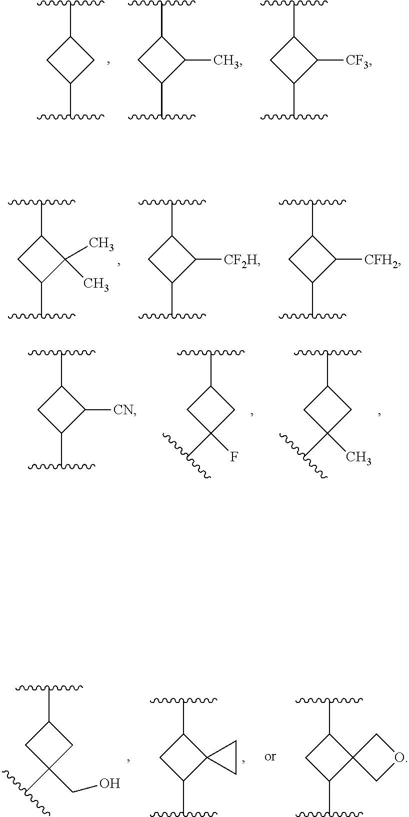

In some cases,

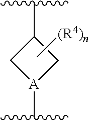

is

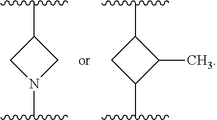 [SMILES: *C1CN(*)C1 ; *C1CC(*)C1C]

In some cases, W is CH. In some cases, W is C-halo (e.g., C—F, C—Cl, or C—Br) or C—CN. In some cases, W is C—F, C—Cl, or C—CN. In some cases, W is C—C₁₋₃alkyl or C—C₁₋₂haloalkyl. In some cases, W is C—CH₃, C—CH₂CH₃, C—CH₂CH₂CH₃, C—CH(CH₃)₂, C—CF₃, C—CHF₂, or C—CH₂F. In some cases, W is C—CH, or C—CH₂CH₃. In some cases, W is C—C₀₋₃alkyleneOH or C—C₀₋₃alkylene-C₁₋₄alkoxy. In some cases, W is C—OH, C—CH₂OH, C—CH₂CH₂OH, C—OCH₃, C—CH₂OCH₃, or C—CH₂CH₂OCH₃, In some cases, W is C—OH, C—CH₂OH, C—OCH₃, or C—CH₂OCH₃. In some cases. W is CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, W is CH, C—F, C—Cl, C—CN, C—CH₃, C—CH₂CH₃, C—OH, C—CH₂OH, C—OCH₃, or C—CH₂OCH₃. In some cases, X is N. In some cases, W is CH and X is N. In some cases, X is C—R^{5a}. In some cases, W is CH and X is C—R^{5a}. In some cases, R^{5a} is H. In some cases, R^{5a} is CN. In some cases, R^{5a} is Br, Cl, or F. In some cases, R^{5a} is C₁₋₃alkyl or C₁₋₃haloalkyl. In some cases, R^{5a} is CH₃, CH₂CH₃, CF₃, CHF₂, or CH₂F. In some cases, R^{5a} is CH₃. In some cases, R^{5a} is C₂₋₆alkenyl or C₂₋₆alkynyl. In some cases, R^{5a} is C₂₋₃alkenyl or C₂₋₃alkynyl. In some cases, R^{5a} is

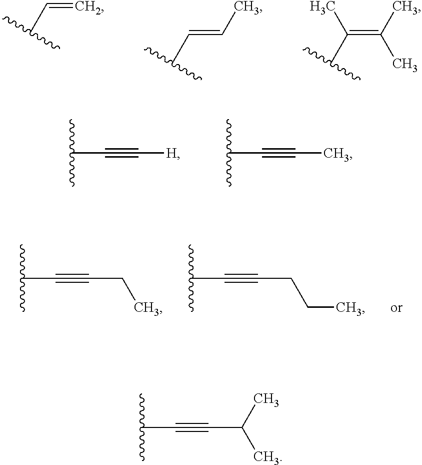

In some cases, R^{5a} is

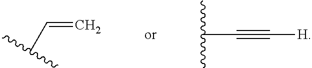

In some cases, R^{5a} is C₀₋₃alkyleneOH or C₀₋₃alkylene-C₁₋₃alkoxy. In some cases, R^{5a} is OH, CH₂OH, OCH₃, or CH₂OCH₃. In some cases, R^{5a} is cycloalkyl having 3-5 total ring atoms. In some cases, R^{5a} is

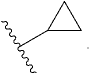 [SMILES: *C1CC1]

In some cases, R^{5a} is H, CN, Br, Cl, F, CH₃, CH₂CH₃, CF₃, CHF₂, CH₂F,

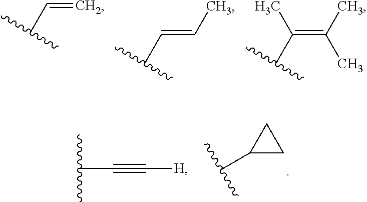

OH, CH₂OH, OCH₃, CH₂OCH₃, or

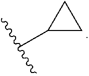 [SMILES: *C1CC1]

In some cases, R^{5a} is H, CN, or CH₃.

In some cases, R^{5b} is C₁₋₃haloalkyl. In some cases, R^{5b} is CF₃, CF₂H, CFH₂, or CF₂CH₃. In some cases, R^{5b} is CF₃. In some cases, R^{5b} is CF₂H. In some cases, R^{5b} is CHF₂. In some cases, R^{5b} is halo. In some cases, R^{5b} is Br, Cl, or F. In some cases, R^{5b} is C₁₋₃alkoxy or C₁₋₃thioalkoxy. In some cases, R^{5b} is OCH₃, OCH₂CH₃, SCH₃, or SCH₂CH₃. In some cases, R^{5b} is OCH₃, or SCH₃. In some cases, R^{5b} is C₁₋₆alkyl (e.g., CH₃, CH₂CH₃, CH₂CH₂CH₃, CH(CH₃)₂), C₂₋₄alkenyl (e.g., CH═CH₂, CH═CHCH₃), or C₂₋₄alkynyl (e.g., CCH, CCH₃), each of which is either unsubstituted or substituted with 1 or more substituents. In some cases, the C₁₋₆alkyl, C₂₋₄alkenyl, or C₂₋₄alkynyl is unsubstituted. In some cases, R^{5b} is CH₃, CH₂CH₃, CH₂CH₂CH₃, or CH(CH₃)₂. In some cases, the C₁₋₆alkyl, C₂₋₄alkenyl, or C₂₋₄alkynyl is substituted with 1, 2, or 3 substituents. In some cases, each of the 1, 2, or 3 substituents independently is C₁₋₃haloalkyl, C₀₋₆alkylene (OH), C₀₋₆alkylene-C₁₋₃alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 3-7 total ring atoms and 1-3 heteroatoms selected from N, O, and S, or phenyl. In some cases, each of the 1, 2, or 3 substituents independently is CH₃, CF₃, CF₂H, CFH₂, OH, OCH₃, OCF₃, CH₂OH, CH₂OCH₃, cyclopropyl, cyclobutyl, or phenyl. In some cases, R^{5b} is cycloalkyl having 3-7 total ring atoms (e.g., cyclopropyl, cyclobutyl, cyclopentyl, or cyclohexyl), cycloalkenyl having 5-7 total ring atoms (e.g., cyclopentenyl or cyclohexenyl), heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is optionally substituted with 1, 2, or 3 substituents independently selected from halo, C₁₋₃alkyl, C₁₋₃haloalkyl, C₀₋₆alkylene(OH), or C₀₋₆alkylene-C₁₋₃alkoxy. In some cases, the heterocycloalkyl group is aziridinyl, oxiranyl, thiranyl, azetidinyl, oxetanyl, thietanyl, pyrrolidinyl, imidazolidinyl, pyrazolidinyl, tetrahydrofuranyl, tetrahydrothiopheneyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothipyranyl, dithianyl, morpholinyl, or thiomorpholinyl. In some cases, the heterocycloalkenyl group is dihydropyrrolyl, dihydrofuranyl, dihydrothiopheneyl, dihydroisoxazolyl, tetrahydropyridinyl, dihydropyranyl, or dihydrothipyranyl. In some cases, R^{5b} is cyclopropyl, cyclobutyl, cyclopentenyl, oxetanyl, or tetrahydrofuranyl. In some cases, R^{5b} is CF₃, CF₂H, CFH₂, CH₃, CH₂CH₃, CH₂CH₂CH₃, CH(CH₃)₂,

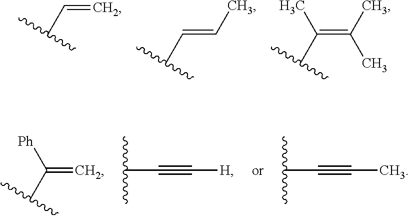

In some cases, R^{5a} and R^{5b}, together with the atoms to which they are attached, form a fused cycloalkyl ring having 3-7 total ring atoms. In some cases, R^{5a} and R^{5b}, together with the atoms to which they are attached, form fused-cyclopropyl, fused-cyclobutyl, or fused-cyclopentyl. In some cases, W is CH; X is N; and R^{5b} is CF₃ or CF₂H. In some cases,

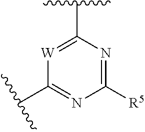

is

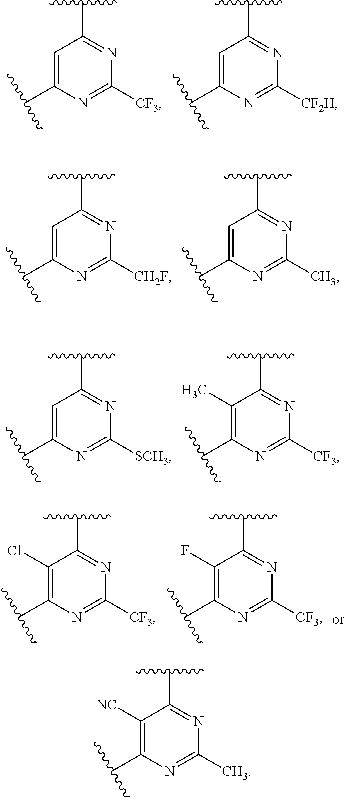

In some cases,

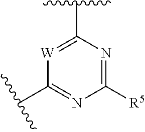

is

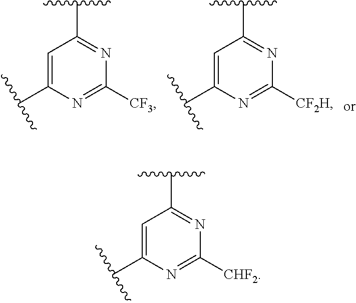 [SMILES: *c1cc(*)nc(C(F)(F)F)n1 ; *c1cc(*)nc(C(F)F)n1 ; *c1cc(*)nc(C(F)F)n1]

In some cases, W is CH; X is C—R^{5a}; R^{5a} is CN, Br, Cl, F, or CH₃; and R^{5b} is CF₃, CF₂H, or CFH₂. In some cases,

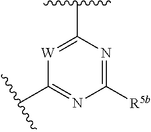

is

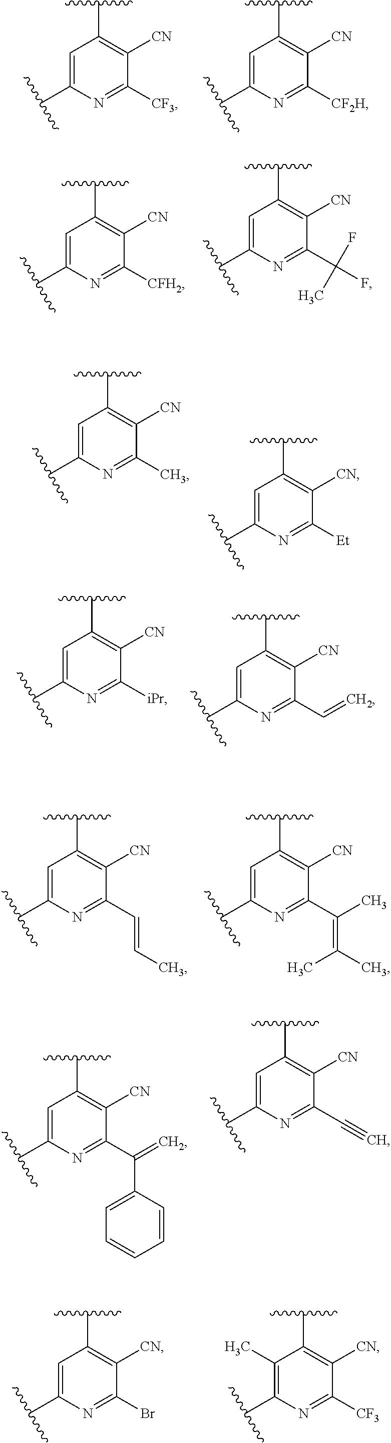

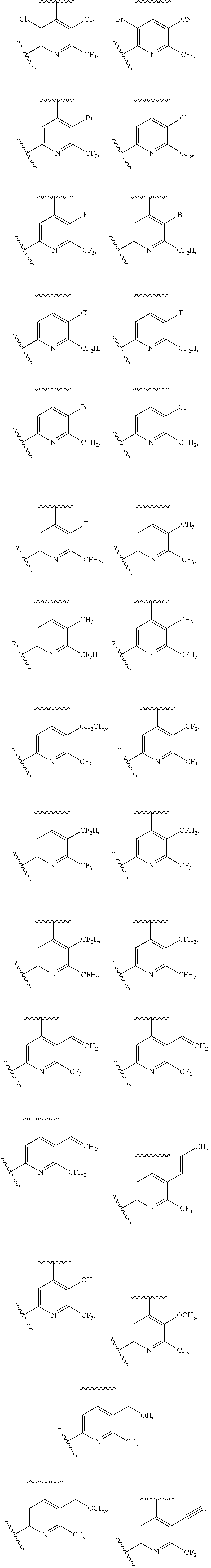

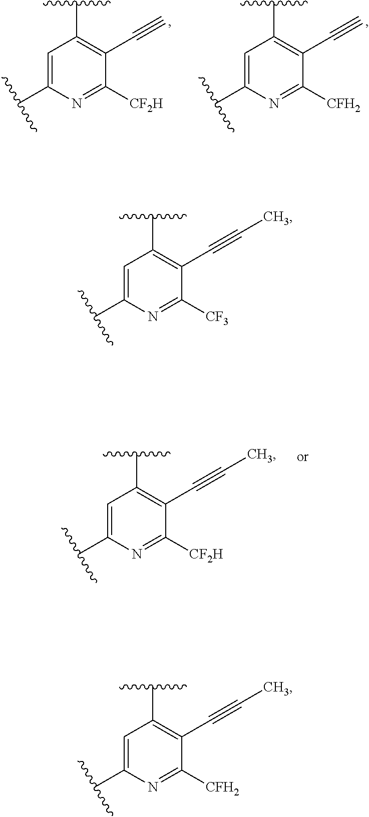

In some cases,

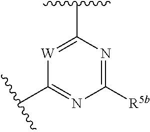

is

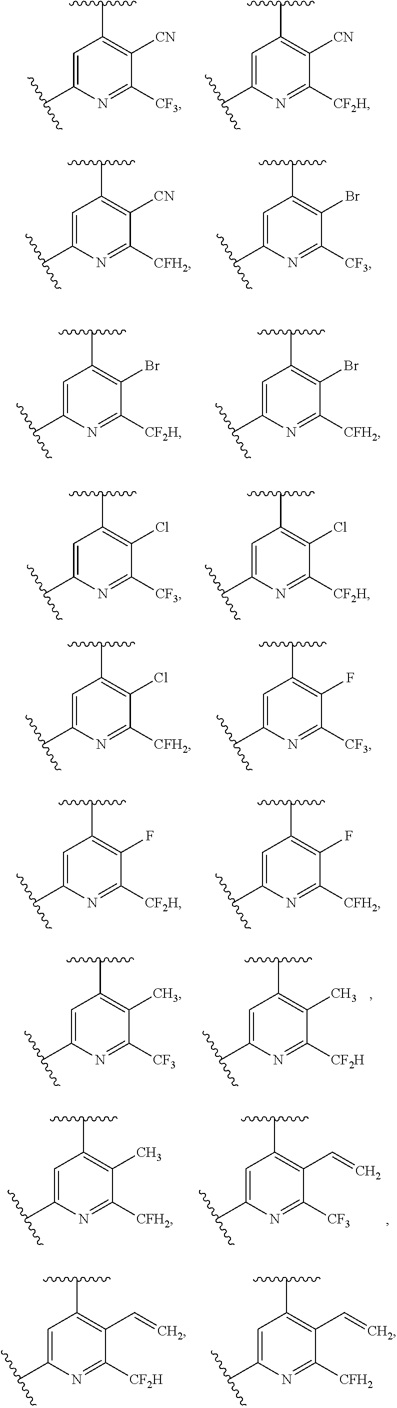

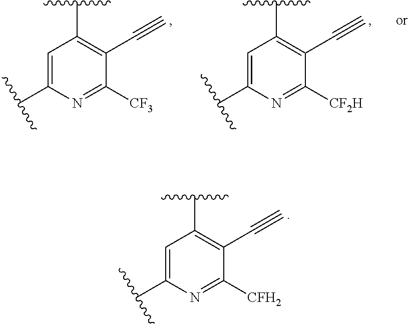

In some cases,

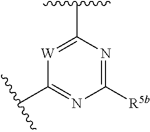

is

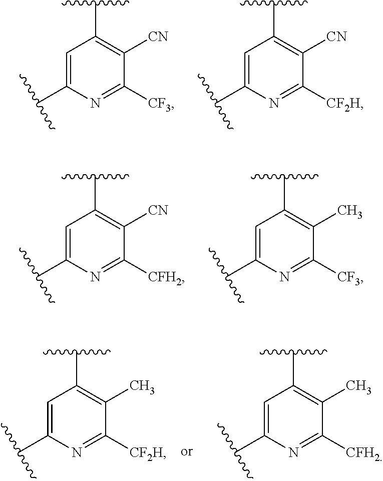

In some cases, o is 0. In some cases, o is 1. In some cases, o is 2. In some cases, o is 3. In some cases, o is 4. In some cases, at least one R⁶ is halo or CN. In some cases, at least one R⁶ is Br, Cl, F, or CN. In some cases, at least one R⁶ is F. In some cases, o is 1 or 2 and each R⁶ independently is F. In some cases, at least one R⁶ is C₁₋₃alkyl or C₁₋₃haloalkyl. In some cases, at least one R⁶ is CH₃, CH₂F, CHF₂, or CF₃. In some cases, o is 1 or 2 and each R⁶ independently is CH₃. In some cases, at least one R⁶ is C₀₋₃alkyleneOH, C₀₋₃alkylene-C₁₋₃alkoxy, deuterated C₀₋₃alkylene-C₁₋₃alkoxy, or C₁₋₄alkylene-N(R^{N1})₂, and each R^{N1} independently is H or CH₃. In some cases, each R^{N1} independently is H. In some cases, at least one R⁶ is OH, CH₂OH, CH₂CH₂OH, OCH₃, OCD₃, CH₂OCH₃, or CH₂CH₂OCH₃. In some cases, at least one R⁶ is CH₂N(CH₃)₂, CH₂NH(CH₃), or CH₂NH₂. In some cases, at least one R⁶ is OH, CH₂OH, OCH₃, OCD₃, CH₂OCH₃, or CH₂N(CH₃)₂. In some cases, o is 1 and R⁶ is OH, CH₂OH, OCH₃, or CH₂OCH₃. In some cases, two geminal R⁶ form an oxo group (═O). In some cases, two geminal R⁶ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S. In some cases, two geminal R⁶ together with the atom to which they are attached form spiro-cyclopropyl, spiro-cyclobutyl, spiro-oxetanyl, or spiro-tetrahydrofuranyl. In some cases, two geminal R⁶ together with the atom to which they are attached form spiro-cyclopropyl. In some cases, two adjacent R⁶, together with the atoms to which they are attached, form a fused cycloalkyl ring having 3-7 total ring atoms. In some cases, two adjacent R⁶, together with the atoms to which they are attached, form a fused-cyclopropyl ring, a fused-cyclobutyl ring, or a fused-cyclopentyl ring. In some cases, the spiro-cycloalkyl ring, the spiro-heterocycloalkyl ring, and the fused-cycloalkyl ring is unsubstituted. In some cases, the spiro-cycloalkyl ring, the spiro-heterocycloalkyl ring, and the fused-cycloalkyl ring is substituted with 1 or more substituents. In some cases, the spiro-cycloalkyl ring, the spiro-heterocycloalkyl ring, and the fused-cycloalkyl ring is substituted with 1, 2, 3, or 4 substituents. In some cases, the spiro-cycloalkyl ring, the spiro-heterocycloalkyl ring, and the fused-cycloalkyl ring is substituted with 1 or 2 substituents. In some cases, the one or more (e.g., 1, 2, or 3 substituents or 1 or 2 substituents) are each independently halo, OH, C₁₋₃alkoxy (e.g., OCH₃, OCH₂CH₃), or CN. In some cases, two non-adjacent R⁶ join together to form a C₁₋₃alkylene bridge or a C₁₋₃ether bridge. In some cases, two non-adjacent R⁶ join together to form a C₁alkylene bridge

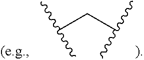 [SMILES: *C*]

In some cases, two non-adjacent R⁶ join together to form a C₂alkylene bridge

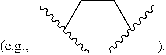 [SMILES: *CC*]

In some cases, two non-adjacent R⁶ join together to form a C₃alkylene bridge

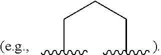 [SMILES: *CCC*]

In some cases, two non-adjacent R⁶ join together to form a C₁₋₃ ether bridge

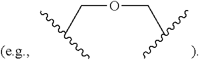 [SMILES: *COC*]

In some cases,

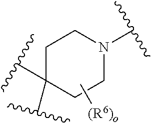

is

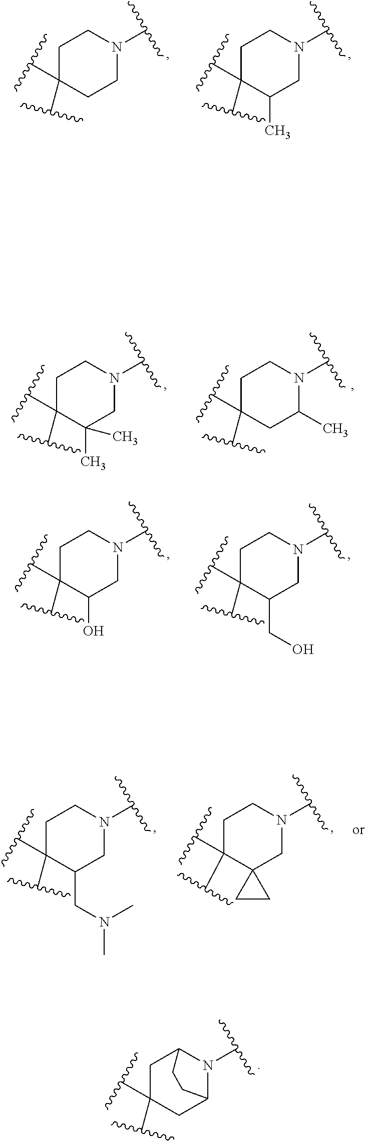

In some cases,

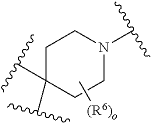

is

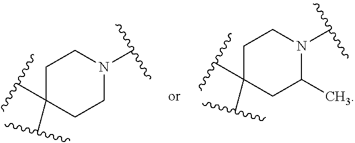 [SMILES: *N1CCC(*)(*)CC1 ; *N1CCC(*)(*)CC1C]

In some cases, Y′ is cycloalkyl having 3 to 7 total ring atoms or cycloalkenyl having 4 to 7 total ring atoms. In some cases, Y′ is cyclopropyl, cyclobutyl, cyclopentyl, cyclohexyl, cyclopentenyl, or cyclohexenyl. In some cases, Y′ is heterocycloalkyl having 3 to 7 total ring atoms and 1, 2, 3 heteroatoms selected from N, O, and S; or heterocycloalkenyl having 5 to 7 total ring atoms and 1, 2, or 3 heteroatoms selected from N, O, and S. In some cases, Y′ is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothipyranyl, dithianyl, morpholinyl, thiomorpholinyl, dihydropyrrolyl, dihydrofuranyl, dihydrothiopheneyl, dihydroisoxazolyl, tetrahydropyridinyl, dihydropyranyl, or dihydrothipyranyl. In some cases, Y′ is azetidinyl, oxetanyl, thietanyl, pyrrolidinyl, imidazolidinyl, tetrahydrofuranyl, oxazolidinyl, isoxazolidinyl, tetrahydropyranyl, dioxanyl, morpholinyl, dihydropyrrolyl, dihydrofuranyl, dihydroisoxazolyl, or dihydropyranyl. In some cases, Y′ is oxetanyl, pyrrolidinyl, tetrahydrofuranyl, dioxolanyl, oxazolidinyl, isoxazolidinyl, tetrahydropyranyl, or morpholinyl. In some cases, Y′ is pyrrolidinyl, tetrahydrofuranyl, tetrahydropyranyl, morpholinyl, dihydrofuranyl, dihydroisoxazolyl, or dihydropyranyl. In some cases, Y′ is pyrrolidinyl, tetrahydrofuranyl, tetrahydropyranyl, or dihydrofuranyl. In some cases, Y′ is pyrrolidinyl. In some cases, Y′ is tetrahydrofuranyl. In some cases, Y′ is tetrahydropyranyl. In some cases, Y′ is morpholinyl. In some cases, Y′ is dihydrofuranyl. In some cases, Y′ is dihydroisoxazolyl. In some cases, Y′ is dihydropyranyl. In some cases, p is 0. In some cases, p is 1. In some cases, p is 2. In some cases, p is 3. In some cases, p is 4. In some cases, p is 5. In some cases, at least one R⁷ is F, Cl, Br, or CN. In some cases, at least one R⁷ is F, Cl, Br, or CN. In some cases, at least one R⁷ is C₁₋₃alkyl, C₁₋₃haloalkyl, or C₂₋₃alkenyl. In some cases, at least one R⁷ is CH₃, CH₂CH₃, CH₂CH₂CH₃, CH(CH₃), CH₂F, CHF₂, or CF₃. In some cases, at least one R⁷ is CH₃, CH₂CH₃, CH₂F, CHF₂, or CF₃. In some cases, at least one R⁷ is CH₃. In some cases, at least one R⁷ is CF₃, CHF₂, or CH₂F. In some cases, at least one R⁷ is C₀₋₃alkyleneOH or C₀₋₃alkylene-C₁₋₃alkoxy. In some cases, at least one R⁷ is OH, OCH₃, OCH₂CH₃, CH₂OH, CH₂OCH₃, or CH₂CH₂OCH₃. In some cases, at least one R⁷ is C(═O)OC₁₋₃alkyl. In some cases, at least one R⁷ is C(═O)OCH₃. In some cases, at least one R⁷ is C₁₋₃alkylene-cycloalkyl having 3-7 total ring atoms. In some cases, the cycloalkyl is cyclopropyl, cyclobutyl, or cyclopentyl. In some cases, at least one R⁷ is cyclopropyl, cyclobutyl, cyclopentyl, CH₂-cyclopropyl, CH₂-cyclobutyl, or CH₂-cyclopentyl. In some cases, at least one R⁷ is C₀₋₄alkylene-heterocycloalkyl having 3-7 total ring atoms and 1, 2, or 3 heteroatoms selected from N, O, and S. In some cases, the heterocycloalkyl is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothipyranyl, dithianyl, morpholinyl, or thiomorpholinyl. In some cases, the heterocycloalkyl is azetidinyl, oxetanyl, pyrrolidinyl, pyrazolidinyl, tetrahydrofuranyl, tetrahydropyranyl, or piperidinyl. In some cases, at least one R⁷ is oxetanyl, pyrrolidinyl, or CH₂-pyrrolidinyl. In some cases, at least one R⁷ is C₀₋₄alkylene-aryl having 6-10 total ring atoms. In some cases, the aryl is phenyl. In some cases, at least one R⁷ is phenyl or CH₂-phenyl. In some cases, at least one R⁷ is C₀₋₄alkylene-heteroaryl 1 having 5-7 total ring atoms and 1, 2, or 3 heteroatoms selected from N, O, and S. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, the heteroaryl is pyrrolyl, furanyl, thiophenyl, pyrazolyl, imidazolyl, thiazolyl, isothiazolyl, oxazolyl, isoxazolyl, or pyridyl. In some cases, the heteroaryl is thiophenyl. In some cases, the heteroaryl is pyrazolyl. In some cases, the heteroaryl is thiazolyl. In some cases, the heteroaryl is pyridyl. In some cases, at least one R⁷ is C₀₋₃alkylene-cyclopropyl, C₀₋₄alkylene-cyclobutyl, C₀₋₄alkylene-cyclopentyl, C₀₋₄alkylene-oxetanyl, C₀₋₄alkylene-pyrrolidinyl, C₀₋₄alkylene-phenyl, or C₀₋₄alkylene-oxadiazolyl. In some cases, two geminal R⁷ form CH₂ or an oxo group (═O). In some cases, two geminal R⁷ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S. In some cases, the spiro-cycloalkyl group is spiro-cyclopropyl, spiro-cyclobutyl, or spiro-cyclopentyl. In some cases, the spiro-heterocycloalkyl is spiro-azetidinyl, spiro-oxetanyl, spiro-thietanyl, or spiro-tetrahydrofuranyl. In some cases, two geminal R⁷ together with the atom to which they are attached from spiro-cyclopropyl, spiro-cyclobutyl, spiro-oxetanyl, or spiro-tetrahydrofuranyl. In some cases, two adjacent R⁷ together with the atoms to which they are attached form a fused cycloalkyl ring having 3-7 total ring atoms or a fused heterocycloalkyl ring having 3-7 total ring atoms and 1, 2, or 3 heteroatoms selected from N, O, and S. In some cases, the fused cycloalkyl ring is fused-cyclopropyl, fused-cyclobutyl, or fused-cyclopentyl. In some cases, two adjacent R⁷ together with the atoms to which they are attached from fused-cyclopropyl or fused-cyclobutyl. In some cases, the fused heterocycloalkyl ring is fused-azetidinyl, fused-oxetanyl, fused-thietanyl, fused-pyrrolidinyl, fused-imidazolidinyl, fused-pyrazolidinyl, fused-tetrahydrofuranyl, or fused-tetrahydrothiophenyl. In some cases, two adjacent R⁷ together with the atoms to which they are attached from the fused-oxetanyl. In some cases, two adjacent R⁷ together with the atoms to which they are attached form a fused aryl ring having 6 total ring atoms. In some cases, two adjacent R⁷ together with the atoms to which they are attached from a fused heteroaryl ring having 5-7 total ring atoms and 1, 2, or 3 heteroatoms selected from N. O, and S. In some cases, the fused heteroaryl is fused-pyrrolyl, fused-furanyl, fused-thiophenyl, fused-pyrazolyl, fused-imidazolyl, fused-thiazolyl, fused-isothiazolyl, fused-oxazolyl, fused-isoxazolyl, fused-triazolyl, fused-thiadiazolyl, fused-oxadiazolyl, fused-pyridyl, fused-pyridazinyl, fused-pyrimidinyl, fused-pyrazinyl, or fused-triazinyl. In some cases, the heteroaryl is fused-pyrrolyl, fused-furanyl, fused-thiophenyl, fused-pyrazolyl, fused-imidazolyl, fused-thiazolyl, fused-isothiazolyl, fused-oxazolyl, fused-isoxazolyl, or fused-pyridyl. In some cases, the fused-heteroaryl is fused-thiazolyl, fused-pyrazolyl, fused-pyridyl, or fused-pyridazinyl. In some cases, the fused-heteroaryl is fused-thiazolyl. In some cases, the fused-heteroaryl is fused-pyrazolyl. In some cases, the fused-heteroaryl is fused-pyridyl. In some cases, the fused-heteroaryl is fused-pyridazinyl. In some cases, two adjacent R⁷ together with the atoms to which they are attached form fused-phenyl, fused-thiophenyl, fused-pyrazolyl, fused-thiazolyl, fused-pyridyl, or fused-pyridazinyl. In some cases, any of the foregoing cycloalkyl, heterocycloalkyl, aryl, or heteroaryl rings is unsubstituted. In some cases, any of the forgoing cycloalkyl, heterocycloalkyl, aryl, or heteroaryl rings is substituted with 1 or more substituents. In some cases, any of the forgoing cycloalkyl, heterocycloalkyl, aryl, or heteroaryl rings is substituted with 1, 2, or 3 substituents. In some cases, each substituent independently is halo, CN, OH, C₁₋₃alkoxy, oxo, C₁₋₃alkyl, or C₁₋₃haloalkyl. In some cases, each substituent independently is halo, CN, oxo, or C₁₋₃alkyl, wherein each substituent independently is F, Cl, Br, CN, oxo, or CH₃.

In some cases,

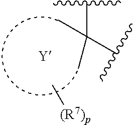

is

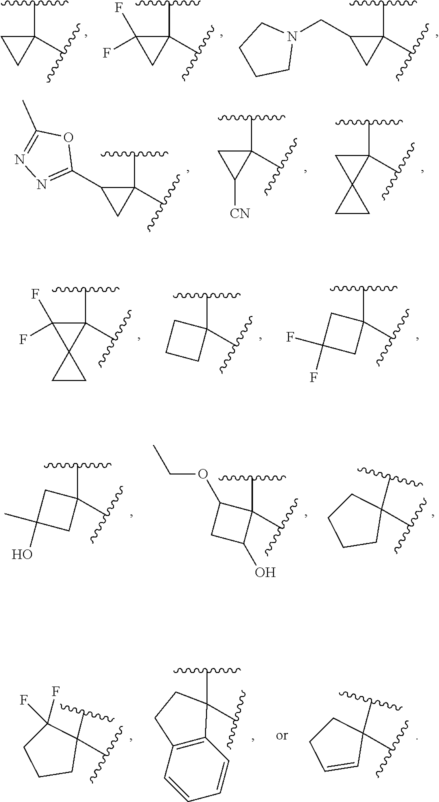

In some cases,

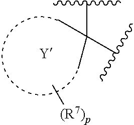

is

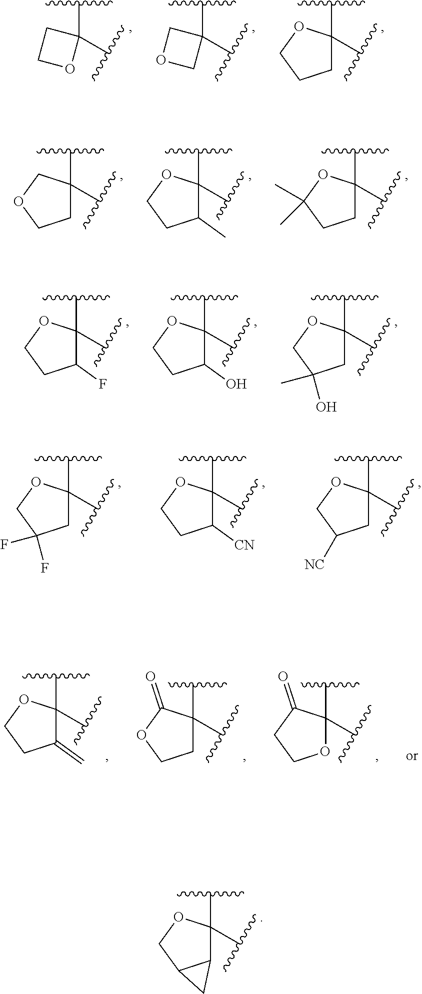

In some cases,

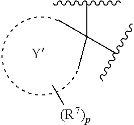

is

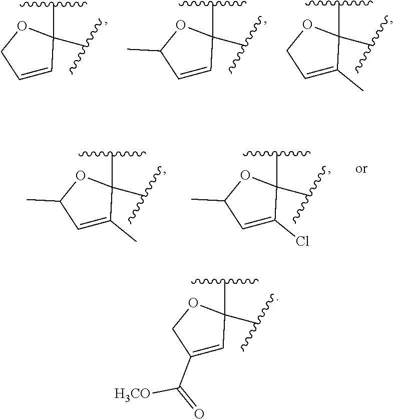

In some cases,

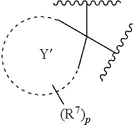

is

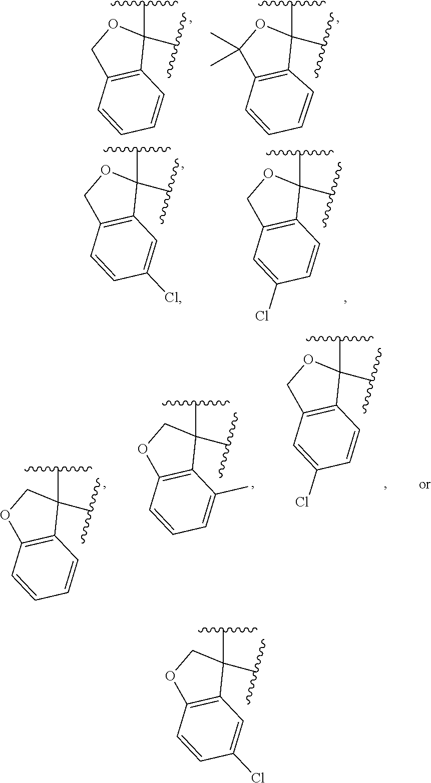

In some cases,

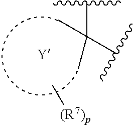

is

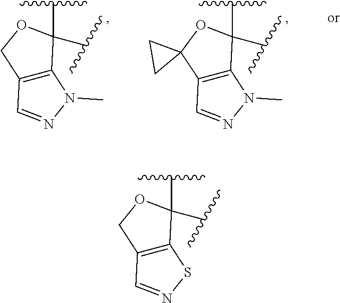 [SMILES: *C1(*)OCc2cnn(C)c21 ; *C1(*)OC2(CC2)c2cnn(C)c21 ; *C1(*)OCc2cnsc21]

In some cases,

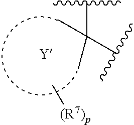

is

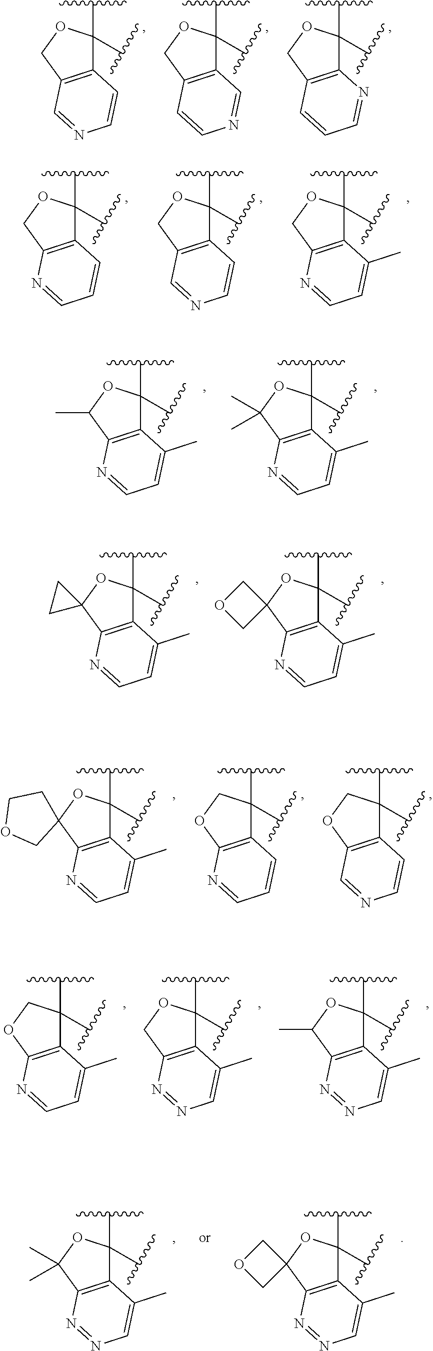

In some cases,

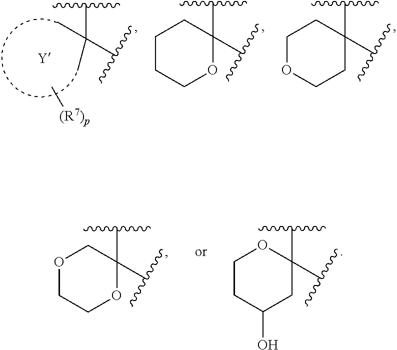

In some cases,

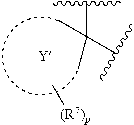

is

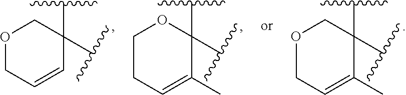

In some cases,

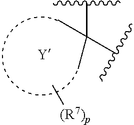

is

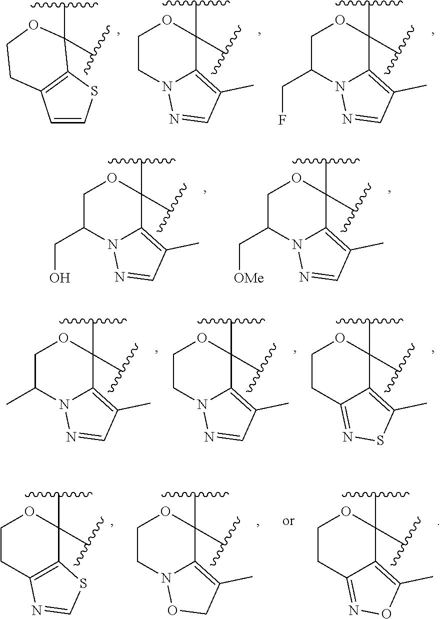

In some cases,

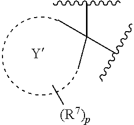

is

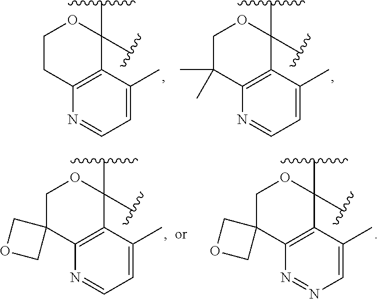

In some cases,

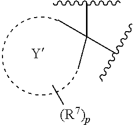

is

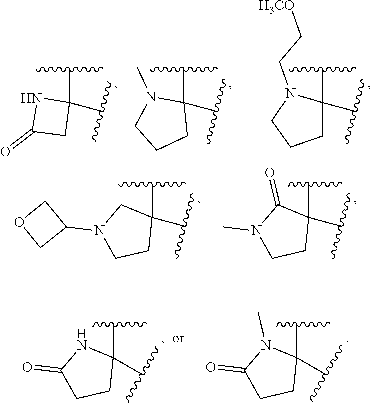

In some cases,

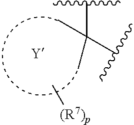

is

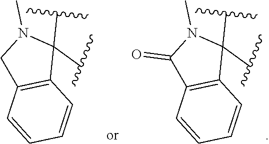 [SMILES: *C1(*)c2ccccc2CN1C ; *C1(*)c2ccccc2C(=O)N1C]

In some cases,

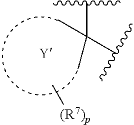

is In some cases,

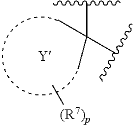

is

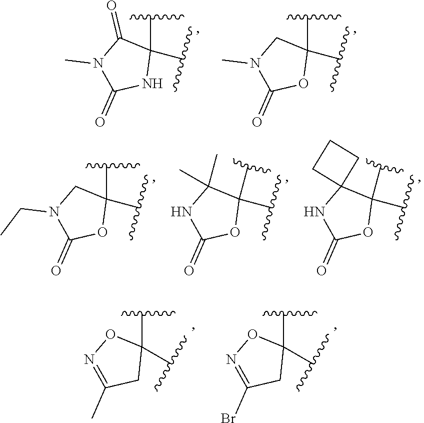

In some cases,

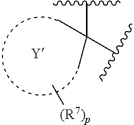

is

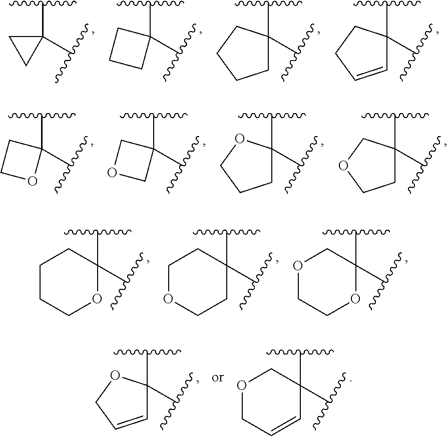

In some cases,

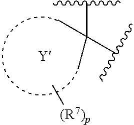

is

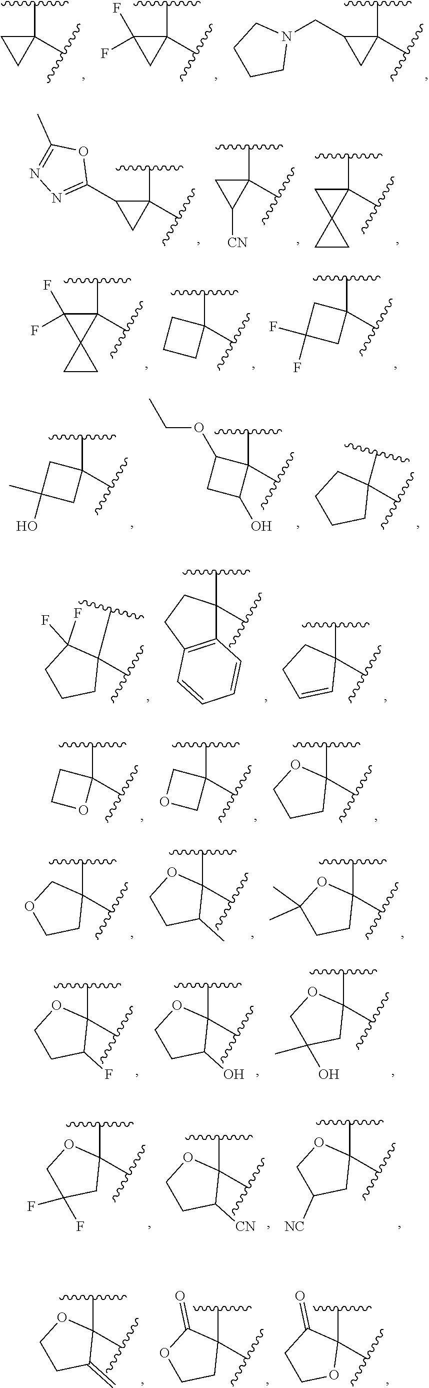

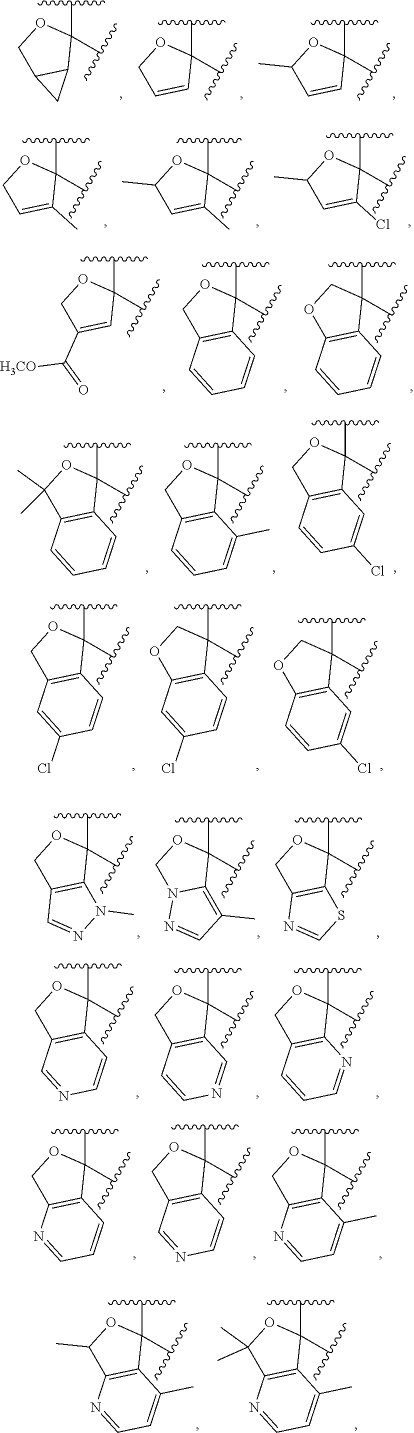

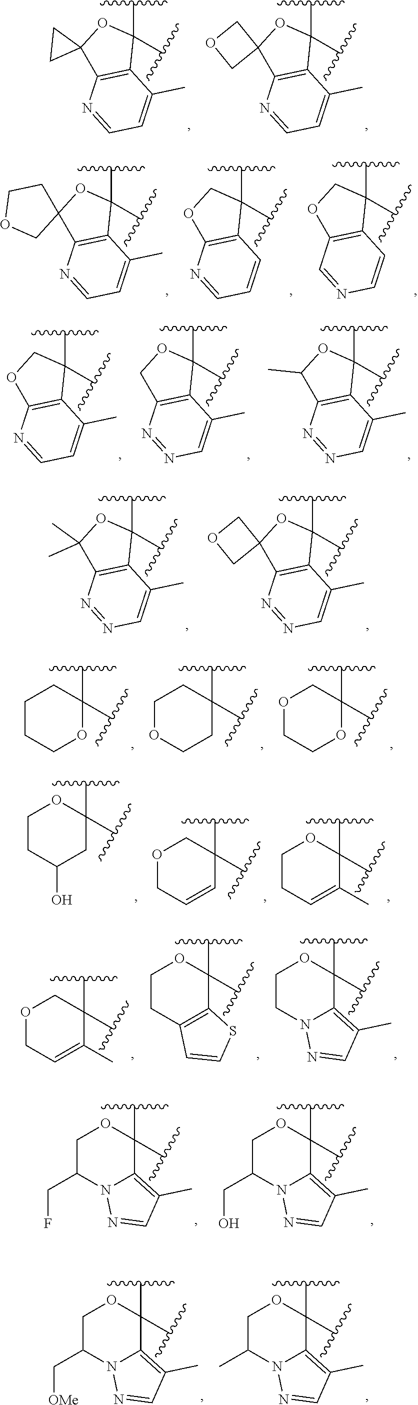

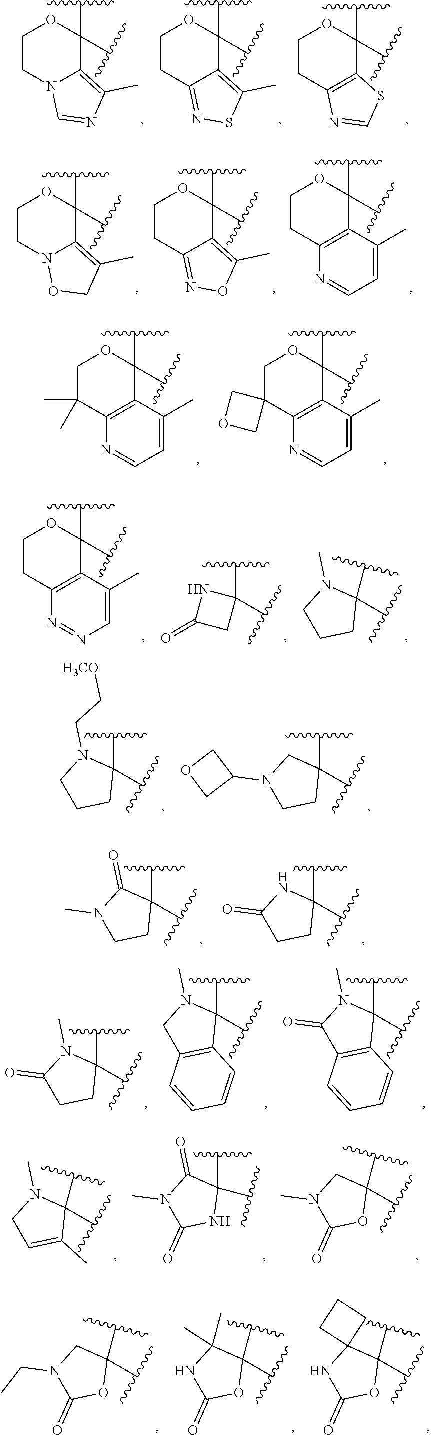

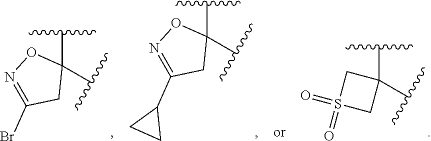 [SMILES: *C1(*)CC(Br)=NO1 ; *C1(*)CC(C2CC2)=NO1 ; *C1(*)CS(=O)(=O)C1]

In some cases,

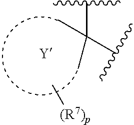

is

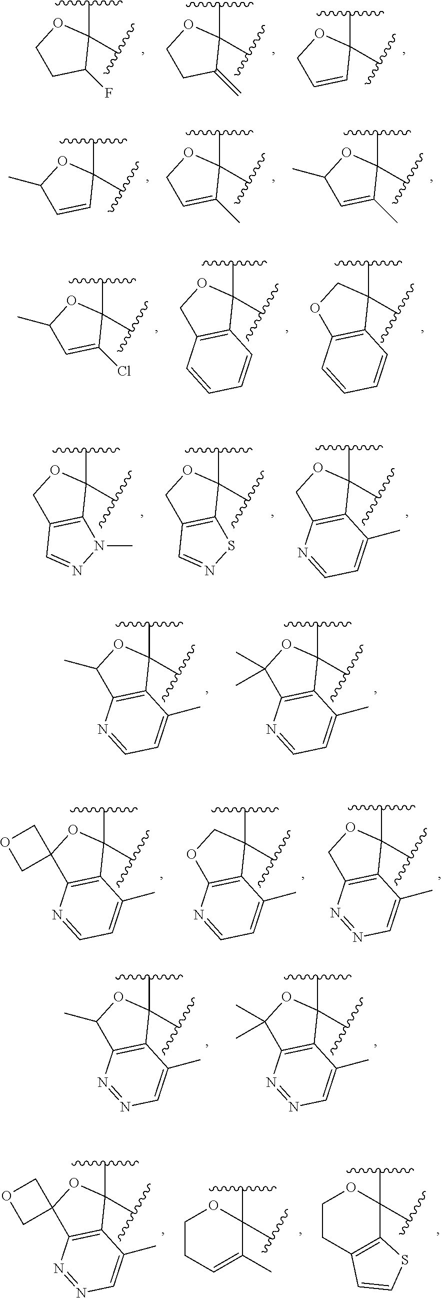

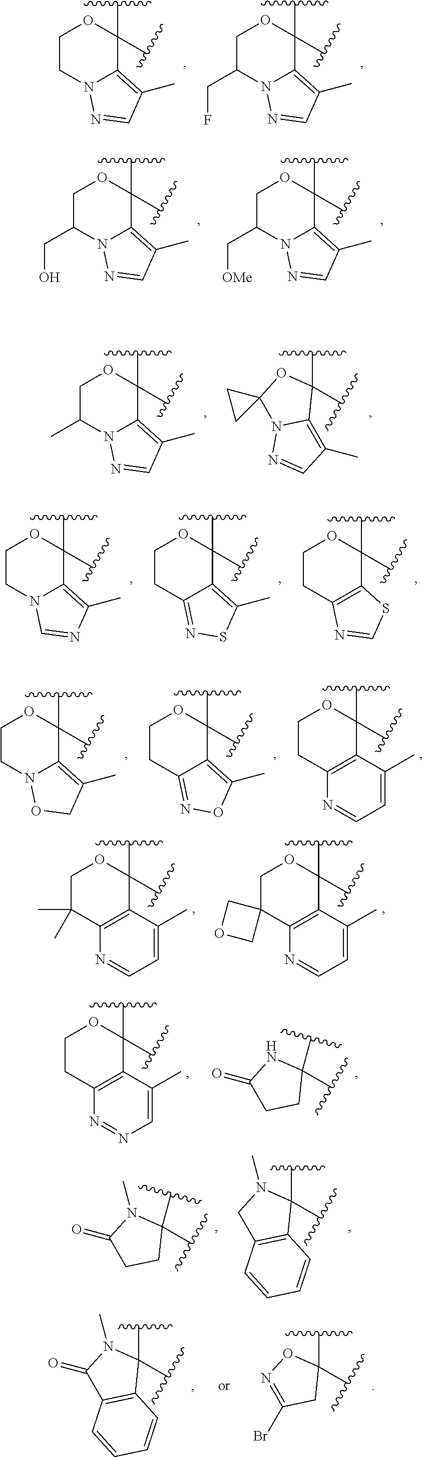

In some cases,

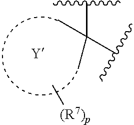

is

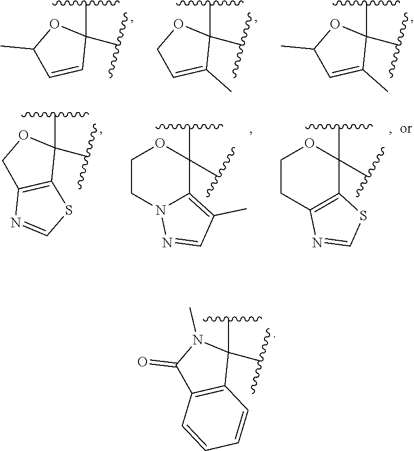

In some cases,

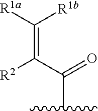

### of Formula (I) is

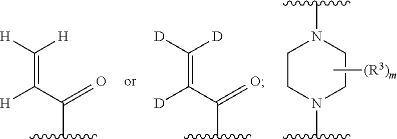

is

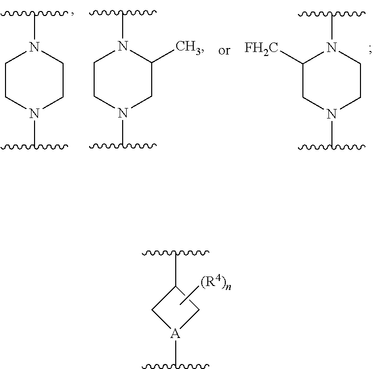

is

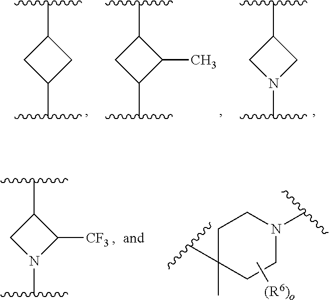

is

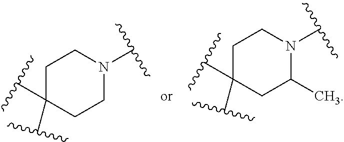 [SMILES: *N1CCC(*)(*)CC1 ; *N1CCC(*)(*)CC1C]

In some cases,

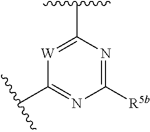

### of Formula (I) is

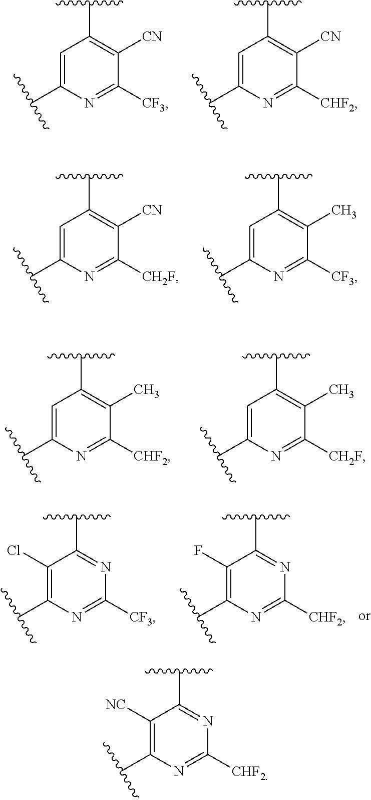

In some cases,

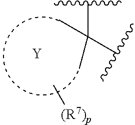

### of Formula (I) is

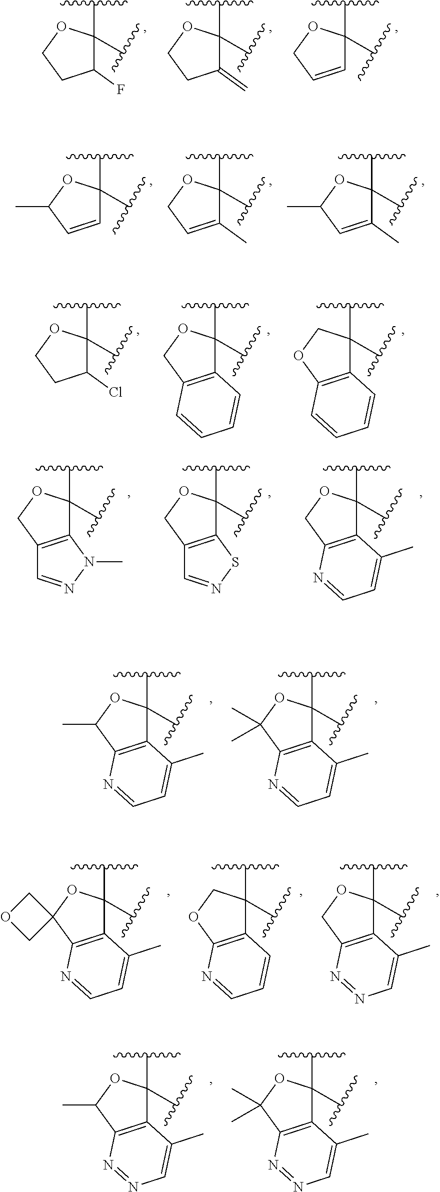

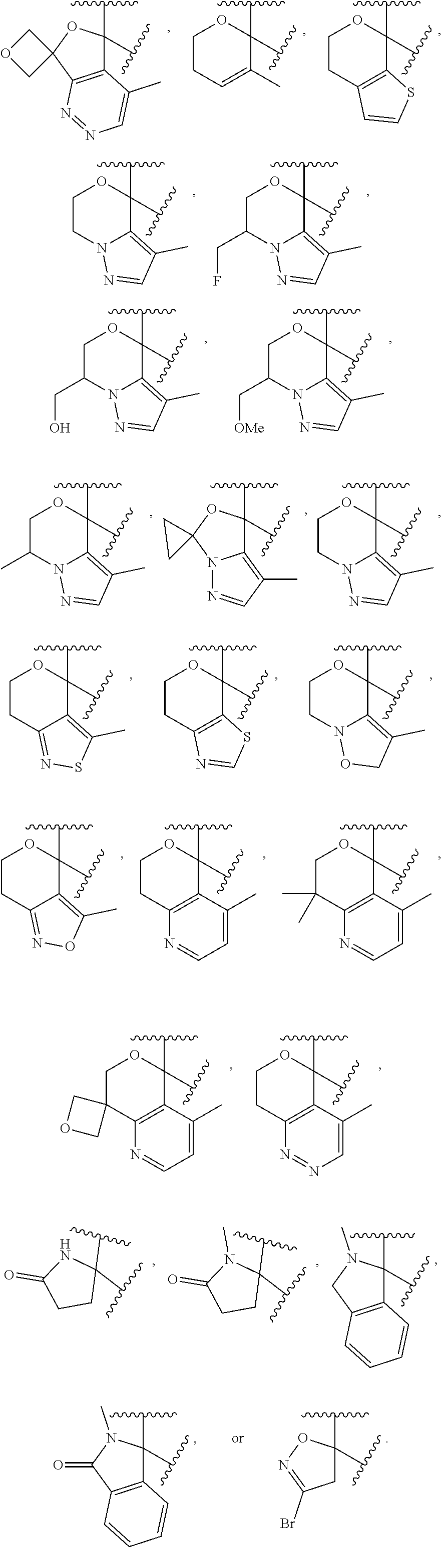

In some cases, the compound of Formula (I) has a structure of Formula (IA) or a pharmaceutically acceptable salt thereof. In some cases, the compound of Formula (I) has a structure of Formula (IB) or a pharmaceutically acceptable salt thereof.

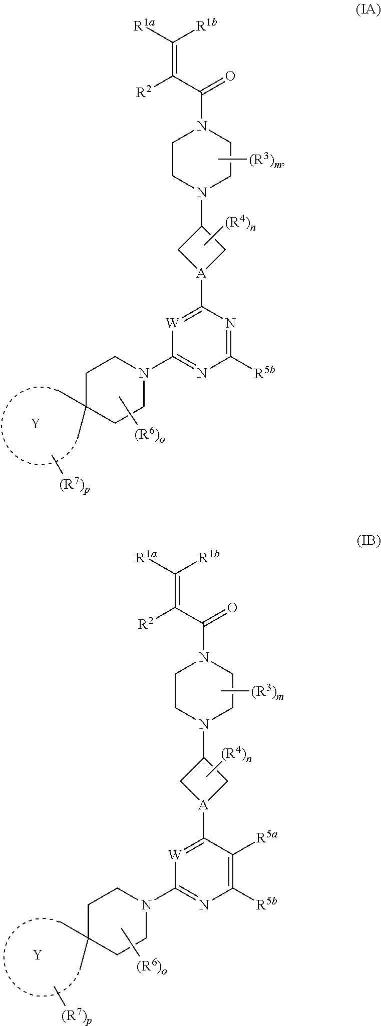

In some cases,

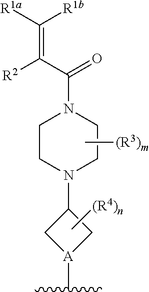

### of Formula (I) is

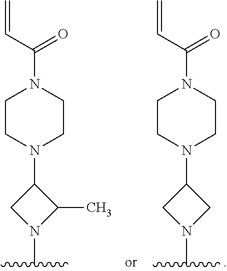 [SMILES: *N1CC(N2CCN(C(=O)C=C)CC2)C1C ; *N1CC(N2CCN(C(=O)C=C)CC2)C1]

In some cases,

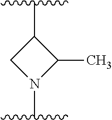 [SMILES: *C1CN(*)C1C]

exhibits the following stereochemical configuration:

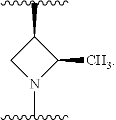 [SMILES: *[C@@H]1CN(*)[C@@H]1C]

In some cases,

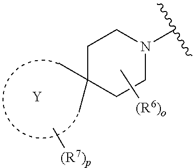

is

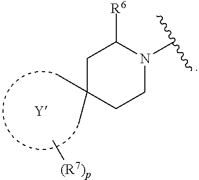

In some cases,

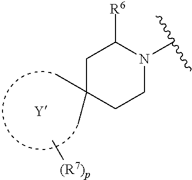

exhibits the following stereochemical configuration:

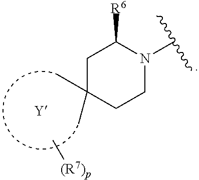

In some cases, R⁶ is CH₃.

It is understood that selections of values of each variable are those that result in the formation of stable or chemically feasible compounds.

### Contemplated Compounds of Formulae (I) and (II)

Specific contemplated compounds of Formulae (I) and (II) include, but are not limited to, compounds in the following Tables, and pharmaceutically acceptable salts thereof.

In some cases, the compound of Formula (I) or Formula (II) is a compound as listed in Table A, or a pharmaceutically acceptable salt thereof:

TABLE A

| Chemical Structure | Name |
|---|---|
| 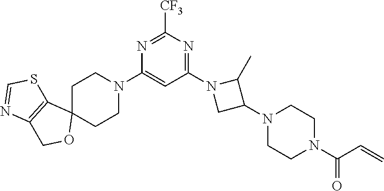 [SMILES: C=CC(=O)N1CCN(C2CN(c3cc(N4CCC5(CC4)OCc4ncsc45)nc(C(F)(F)F)n3)C2C)CC1] | 1-(4-(1-(6-(4H-spiro[furo[3,4-d]thiazole-6,4′- piperidin]-1′-yl)-2-(trifluoromethyl)pyrimidin-4- yl)-2-methylazetidin-3-yl)piperazin-1-yl)prop-2- en-1-one |
| 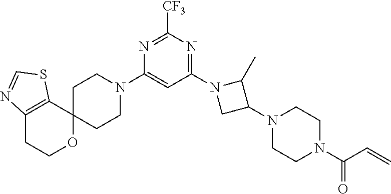 [SMILES: C=CC(=O)N1CCN(C2CN(c3cc(N4CCC5(CC4)OCCc4ncsc45)nc(C(F)(F)F)n3)C2C)CC1] | 1-(4-(1-(6-(6′,7′-dihydrospiro\| piperidine-4,4′- pyrano[4,3-d]thiazol]-1-yl)-2- (trifluoromethyl)pyrimidin-4-yl)-2-methylazetidin- 3-yl)piperazin-1-yl)prop-2-en-1-one |
| 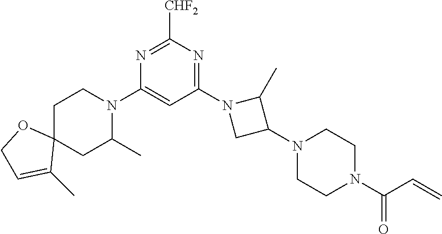 [SMILES: C=CC(=O)N1CCN(C2CN(c3cc(N4CCC5(CC4C)OCC=C5C)nc(C(F)F)n3)C2C)CC1] | 1-(4-(1-(2-(difluoromethyl)-6-(4,7-dimethyl-1-oxa- 8-azaspiro[4.5]dec-3-en-8-y])pyrimidin-4-yl)-2- methylazetidin-3-yl)piperazin-1-yl)prop-2-en-1- one |
| 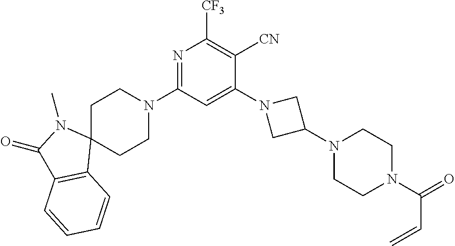 [SMILES: C=CC(=O)N1CCN(C2CN(c3cc(N4CCC5(CC4)c4ccccc4C(=O)N5C)nc(C(F)(F)F)c3C#N)C2)CC1] | 4-(3-(4-acryloylpiperazin-1-yl)azetidin-1-yl)-6-(2- methyl-3-oxospirofisoindoline-1,4′-piperidin]-1′- yl)-2-(trifluoromethyl)nicotinonitrile |
| 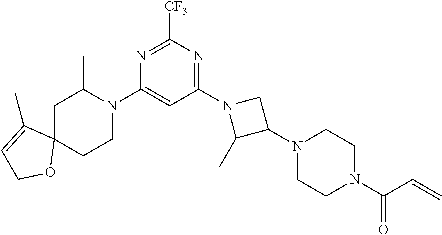 [SMILES: C=CC(=O)N1CCN(C2CN(c3cc(N4CCC5(CC4C)OCC=C5C)nc(C(F)(F)F)n3)C2C)CC1] | 1-(4-(1-(6-(4,7-dimethyl-1-oxa-8-azaspiro[4.5]dec- 3-en-8-yl)-2-(trifluoromethyl)pyrimidin-4-yl)-2- methylazetidin3-yl)piperazin-1-yl)prop-2-en-1-one |
| 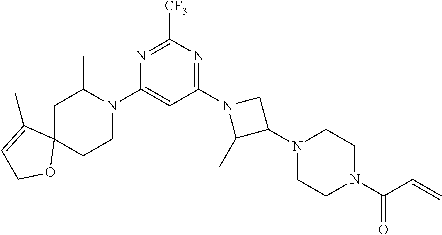 [SMILES: C=CC(=O)N1CCN(C2CN(c3cc(N4CCC5(CC4C)OCC=C5C)nc(C(F)(F)F)n3)C2C)CC1] | 1-(4-(1-(6-(4.7-dimethyl-1-oxa-8-azaspiro[4.5]dec- 3-en-8-yl)-2-(trifluoromethyl)pyrimidin-4-yl)-2- methylazetidin-3-yl)piperazin-1-yl)prop-2-en-1- one |
| 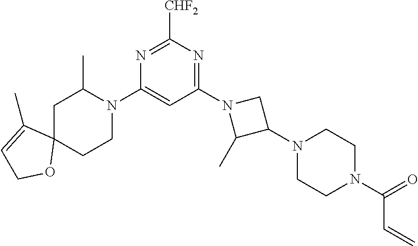 [SMILES: C=CC(=O)N1CCN(C2CN(c3cc(N4CCC5(CC4C)OCC=C5C)nc(C(F)F)n3)C2C)CC1] | 1-(4-(1-(2-(difluoromethyl)-6-(4,7-dimethyl-1-oxa- 8-azaspiro[4.5]dec-3-en-8-yl)pyrimidin-4-yl-2- methylazetidin-3-yl)piperazin-1-yl)prop-2-en-1- one |
| 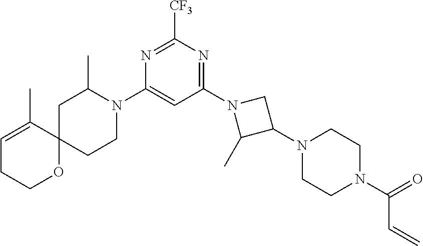 [SMILES: C=CC(=O)N1CCN(C2CN(c3cc(N4CCC5(CC4C)OCCC=C5C)nc(C(F)(F)F)n3)C2C)CC1] | 1-(4-(2-methyl-1-(6-(5-methyl-1-oxa-9- azaspiro[5.5]undec-4-en-9-yl)-2- (trifluoromethyl)pyrimidin-4-yl)azetidin-3- yl)piperazin-1-yl)prop-2-en-1-one |
| 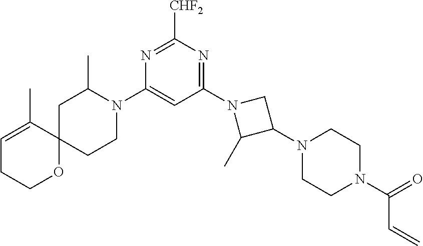 [SMILES: C=CC(=O)N1CCN(C2CN(c3cc(N4CCC5(CC4C)OCCC=C5C)nc(C(F)F)n3)C2C)CC1] | 1-(4-(1-(2-(difluoromethyl)-6-(5-methyl-1-oxa-9- azaspiro[5.5]undec-4-en-9-yl)pyrimidin-4-yl)-2- methylazetidin-3-yl)piperazin-1-yl)prop-2-en-1- one |
| 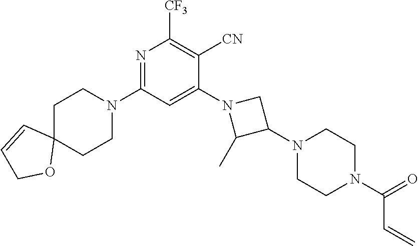 [SMILES: C=CC(=O)N1CCN(C2CN(c3cc(N4CCC5(C=CCO5)CC4)nc(C(F)(F)F)c3C#N)C2C)CC1] | 4-(3-(4-acryloylpiperazin-1-yl)-2-methylazetidin- 1-yl)-6-(1-oxa-8-azaspiro[4.5]dec-3-en-8-yl)-2- (trifluoromethyl)nicotinonitrile |
| 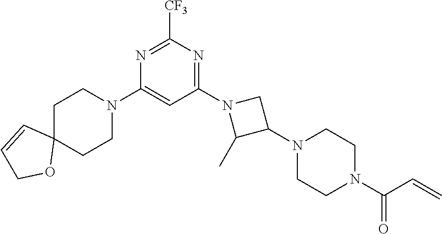 [SMILES: C=CC(=O)N1CCN(C2CN(c3cc(N4CCC5(C=CCO5)CC4)nc(C(F)(F)F)n3)C2C)CC1] | 1-(4-(1-(6-(1-oxa-8-azaspiro[4.5]dec-3-en-8-yl)-2- (trifluoromethyl)pyrimidin-4-yl)-2-methylazetidin- 3-yl)piperazin-1-yl)prop-2-en-1-one |
| 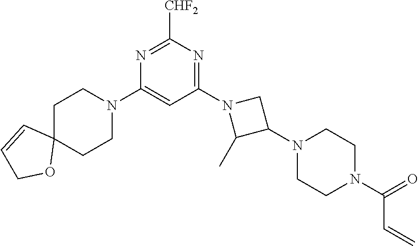 [SMILES: C=CC(=O)N1CCN(C2CN(c3cc(N4CCC5(C=CCO5)CC4)nc(C(F)F)n3)C2C)CC1] | 1-(4-(1-(2-(difluoromethyl)-6-(1-oxa-8- azaspiro[4.5]dec-3-en-8-yl)pyrimidin-4-yl)-2- methylazetidin-3-yl)piperazin-1-yl)prop-2-en-1- one |
| 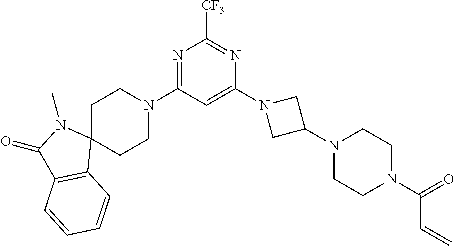 [SMILES: C=CC(=O)N1CCN(C2CN(c3cc(N4CCC5(CC4)c4ccccc4C(=O)N5C)nc(C(F)(F)F)n3)C2)CC1] | 1′-(6-(3-(4-acryloylpiperazin-1-yl)azetidin-1-yl)-2- (trifluoromethyl)pyrimidin-4-yl)-2- methylspiro[isoindoline-1,4′-piperidin]-3-one |
| 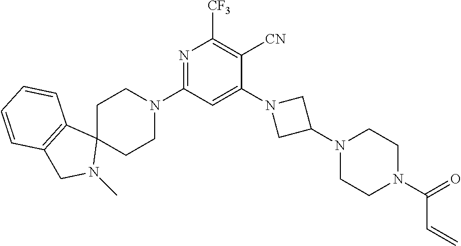 [SMILES: C=CC(=O)N1CCN(C2CN(c3cc(N4CCC5(CC4)c4ccccc4CN5C)nc(C(F)(F)F)c3C#N)C2)CC1] | 4-(3-(4-acryloylpiperazin-1-yl)azetidin-1-yl)-6-(2- methylspirofisoindoline-1,4′-piperidin]-1′-yl)-2- (trifluoromethyl)nicotinonitrile |
| 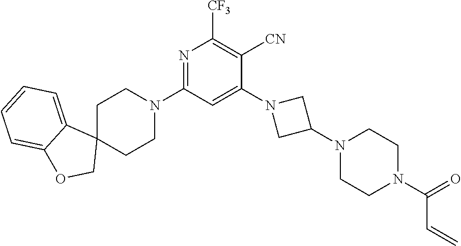 [SMILES: C=CC(=O)N1CCN(C2CN(c3cc(N4CCC5(CC4)COc4ccccc45)nc(C(F)(F)F)c3C#N)C2)CC1] | 4-(3-(4-acryloylpiperazin-1-yl)azetidin-1-yl)-6- (2H-spiro[benzofuran-3,4′-piperidin]-1′-yl)-2- (trifluoromethyl)nicotinonitrile |
| 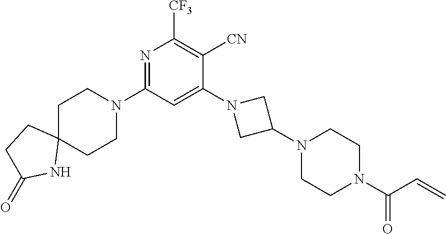 [SMILES: C=CC(=O)N1CCN(C2CN(c3cc(N4CCC5(CCC(=O)N5)CC4)nc(C(F)(F)F)c3C#N)C2)CC1] | 4-(3-(4-acryloylpiperazin-1-yl)azetidin-1-yl)-6-(2- oxo-1,8-diazaspiro[4.5]decan-8-yl)-2- (trifluoromethyl)nicotinonitrile |
| 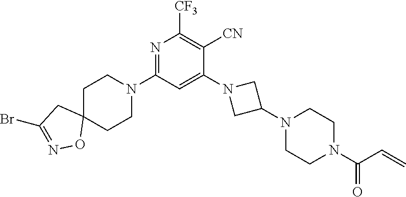 [SMILES: C=CC(=O)N1CCN(C2CN(c3cc(N4CCC5(CC4)CC(Br)=NO5)nc(C(F)(F)F)c3C#N)C2)CC1] | 4-(3-(4-acryloylpiperazin-1-yl)azetidin-1-yl)-6-(3- bromo-1-oxa-2,8-diazaspiro[4.5]dec-2-en-8-yl)-2- (trifluoromethyl)nicotinonitrile |
| 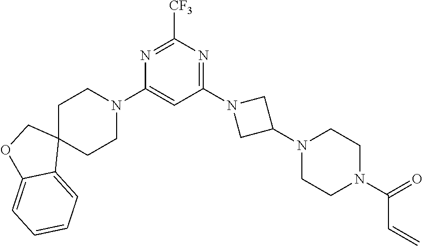 [SMILES: C=CC(=O)N1CCN(C2CN(c3cc(N4CCC5(CC4)COc4ccccc45)nc(C(F)(F)F)n3)C2)CC1] | 1-(4-(1-(6-(2H-spiro[benzofuran-3.4′-piperidin]-1′- yl)-2-(trifluoromethyl)pyrimidin-4-yl)azetidin-3- yl)piperazin-1-yl)prop-2-en-1-one |
| 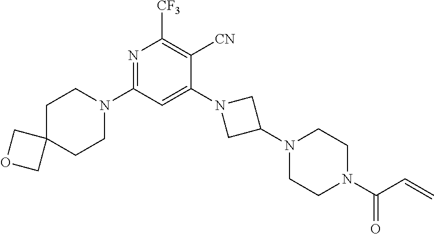 [SMILES: C=CC(=O)N1CCN(C2CN(c3cc(N4CCC5(CC4)COC5)nc(C(F)(F)F)c3C#N)C2)CC1] | 4-(3-(4-acryloylpiperazin-1-yl)azetidin-1-yl)-6-(2- oxa-7-azaspiro[3.5]nonan-7-yl)-2- (trifluoromethyl)nicotinonitrile |
| 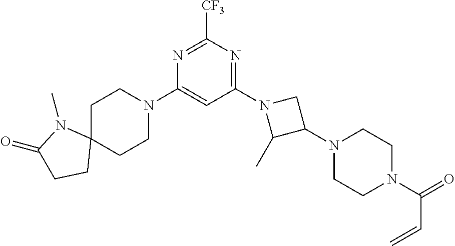 [SMILES: C=CC(=O)N1CCN(C2CN(c3cc(N4CCC5(CCC(=O)N5C)CC4)nc(C(F)(F)F)n3)C2C)CC1] | 8-(6-(3-(4-Acryloylpiperazin-1-yl)-2-methylazeti- din-1-yl)-2-(trifluoromethyl)pyrimidin-4-yl)-1- methyl-1,8-diazaspiro[4.5]decan-2-one |
| 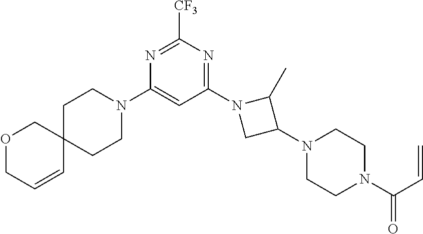 [SMILES: C=CC(=O)N1CCN(C2CN(c3cc(N4CCC5(C=CCOC5)CC4)nc(C(F)(F)F)n3)C2C)CC1] | 1-(4-(1-(6-(2-xxa-9-azaspiro[5.5]undec-4-en-9-yl)- 2-(trifluoromethyl)pyrimidin-4-yl)-2-methyl- azetidin-3-yl)piperazin-1-yl)prop-2-en-1-one |
| 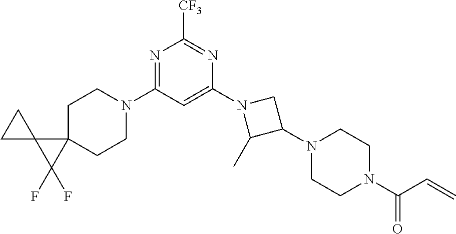 [SMILES: C=CC(=O)N1CCN(C2CN(c3cc(N4CCC5(CC4)C(F)(F)C54CC4)nc(C(F)(F)F)n3)C2C)CC1] | 1-(4-(1-(6-(10.10-difluoro-7- azadispiro[2.0.5⁴.1³]decan-7-yl)-2- (trifluoromethyl)pyrimidin-4-yl)-2-methylazetidin- 3-yl)piperazin-1-yl)prop-2-en-1-one |
| 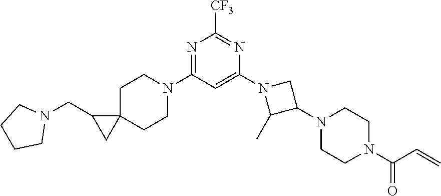 [SMILES: C=CC(=O)N1CCN(C2CN(c3cc(N4CCC5(CC4)CC5CN4CCCC4)nc(C(F)(F)F)n3)C2C)CC1] | 1-(4-(2-methyl-1-(6-(1-(pyrrolidin-1-ylmethyl)-6- azaspiro[2.5]octan-6-yl)-2- (trifluoromethyl)pyrimidin-4-yl)azetidin-3- yl)piperazin-1-yl)prop-2-en-1-one |
| 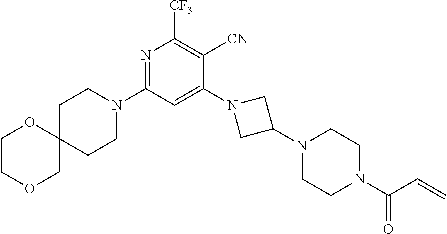 [SMILES: C=CC(=O)N1CCN(C2CN(c3cc(N4CCC5(CC4)COCCO5)nc(C(F)(F)F)c3C#N)C2)CC1] | 4-(3-(4-acryloylpiperazin-1-yl)azetidin-1-yl)-6- (1,4-dioxa-9-azaspiro[5.5]undecan-9-yl)-2- (trifluoromethyl)nicotinonitrile |
| 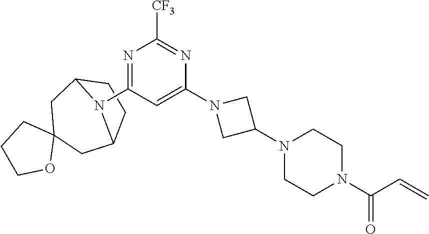 [SMILES: C=CC(=O)N1CCN(C2CN(c3cc(N4C5CCC4CC4(CCCO4)C5)nc(C(F)(F)F)n3)C2)CC1] | 1-(4-(1-(6-(dihydro-3′H-8- azaspiro[bicyclo[3.2.1]octane-3.2′-furan]-8-yl)-2- (trifluoromethyl)pyrimidin-4-yl)azetidin-3- yl)piperazin-1-yl)prop-2-en-1-one |
| 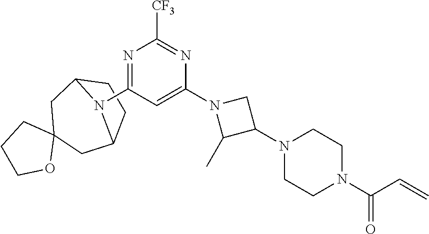 [SMILES: C=CC(=O)N1CCN(C2CN(c3cc(N4C5CCC4CC4(CCCO4)C5)nc(C(F)(F)F)n3)C2C)CC1] | 1-(4-(1-(6-(dihydro-3′H-8- azaspiro[bicyclo[3.2.1]octane-3.2′-furan]-8-yl)-2- (trifluoromethyl)pyrimidin-4-yl)-2-methylazetidin- 3-yl)piperazin-1-yl)prop-2-en-1-one |
| 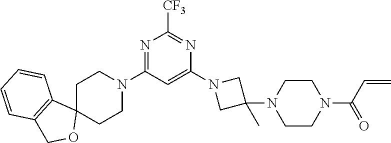 [SMILES: C=CC(=O)N1CCN(C2(C)CN(c3cc(N4CCC5(CC4)OCc4ccccc45)nc(C(F)(F)F)n3)C2)CC1] | 1-(4-(1-(6-(3H-spiro[isobenzofuran-1,4′-piperidin]- 1′-yl)-2-(trifluoromethyl)pyrimidin-4-yl)-3- methylazetidin-3-yl)piperazin-1-yl)prop-2-en-1- one |
| 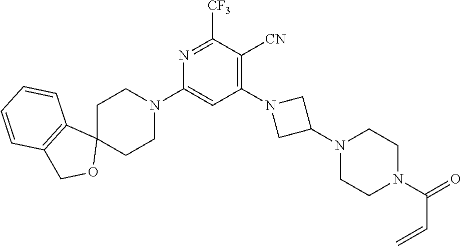 [SMILES: C=CC(=O)N1CCN(C2CN(c3cc(N4CCC5(CC4)OCc4ccccc45)nc(C(F)(F)F)c3C#N)C2)CC1] | 4-(3-(4-acryloylpiperazin-1-yl)azetidin-1-yl)-6- (3H-spiro[isobenzofuran-1,4′-piperidin]-1′-yl)-2- (trifluoromethyl)nicotinonitrile |
| 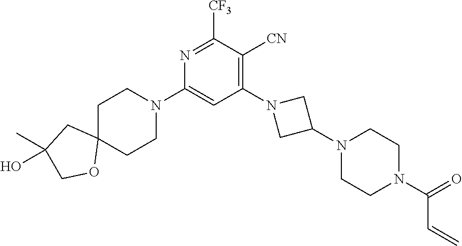 [SMILES: C=CC(=O)N1CCN(C2CN(c3cc(N4CCC5(CC4)CC(C)(O)CO5)nc(C(F)(F)F)c3C#N)C2)CC1] | 4-(3-(4-acryloylpiperazin-1-yl)azetidin-1-yl)-6-(3- hydroxy-3-methyl-1-oxa-8-azaspiro[4.5]decan-8- yl)-2-(trifluoromethyl)nicotinonitrile |
| 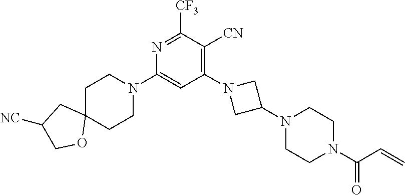 [SMILES: C=CC(=O)N1CCN(C2CN(c3cc(N4CCC5(CC4)CC(C#N)CO5)nc(C(F)(F)F)c3C#N)C2)CC1] | 8-(4-(3-(4-acryloylpiperazin-1-yl)azetidin-1-yl)-5- cyano-6-(trifluoromethyl)pyridin-2-yl)-1-oxa-8- azaspiro[4.5]decane-3-carbonitrile |
| 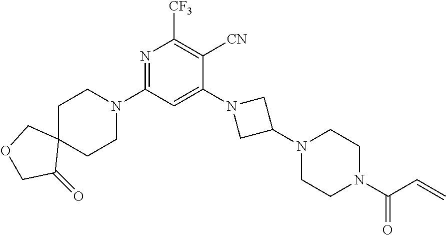 [SMILES: C=CC(=O)N1CCN(C2CN(c3cc(N4CCC5(CC4)COCC5=O)nc(C(F)(F)F)c3C#N)C2)CC1] | 4-(3-(4-acryloylpiperazin-1-yl)azetidin-1-yl)-6-(4- oxo-1-oxa-8-azaspiro[4.5]decan-8-yl)-2- (trifluoromethyl)nicotinonitrile |
| 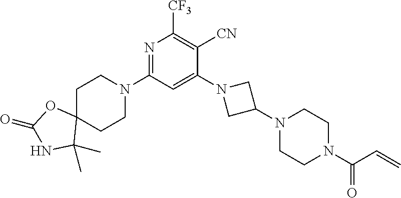 [SMILES: C=CC(=O)N1CCN(C2CN(c3cc(N4CCC5(CC4)OC(=O)NC5(C)C)nc(C(F)(F)F)c3C#N)C2)CC1] | 4-(3-(4-acryloylpiperazin-1-yl)azctidin-1-yl)-6- (4,4-dimethy1-2-oxo-1-oxa-3.8-diazaspiro[4.5]de- can-8-yl)-2-(trifluoromethyl)nicotinonitrile |
| 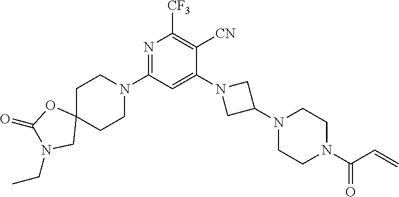 [SMILES: C=CC(=O)N1CCN(C2CN(c3cc(N4CCC5(CC4)CN(CC)C(=O)O5)nc(C(F)(F)F)c3C#N)C2)CC1] | 4-(3-(4-acryloylpiperazin-1-yl)azetidin-1-yl)-6-(3- ethyl-2-oxo-1-oxa-3.8-diazaspiro[4.5]decan-8-yl)- 2-(trifluoromethyl)nicotinonitrile |
| 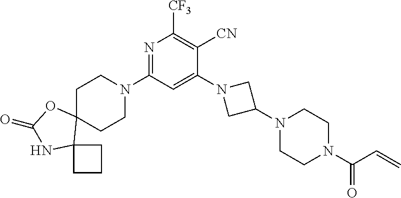 [SMILES: C=CC(=O)N1CCN(C2CN(c3cc(N4CCC5(CC4)OC(=O)NC54CCC4)nc(C(F)(F)F)c3C#N)C2)CC1] | 4-(3-(4-acryloylpiperazin-1-yl)azetidin-1-yl)-6- (12-oxo-11-oxa-8,13-diazadispiro[3.0.55.34]tri- decan-8-yl)-2-(trifluoromethyl)nicotinonitrile |
| 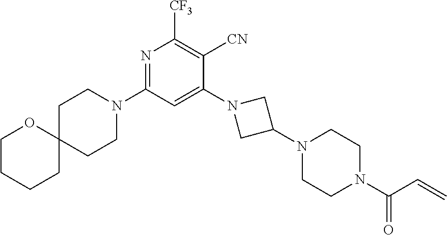 [SMILES: C=CC(=O)N1CCN(C2CN(c3cc(N4CCC5(CCCCO5)CC4)nc(C(F)(F)F)c3C#N)C2)CC1] | 4-(3-(4-acryloylpiperazin-1-yl)azetidin-1-yl)-6- (1-oxa-9-azaspiro[5.5]undecan-9-yl)-2- (trifluoromethyl)nicotinonitrile |
| 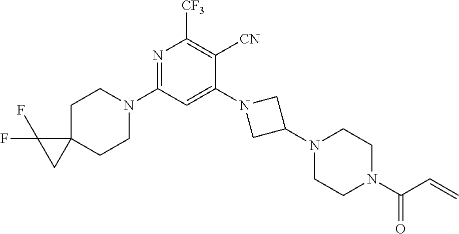 [SMILES: C=CC(=O)N1CCN(C2CN(c3cc(N4CCC5(CC4)CC5(F)F)nc(C(F)(F)F)c3C#N)C2)CC1] | 4-(3-(4-acryloylpiperazin-1-yl)azetidin-1-yl)-6- (1,1-difluoro-6-azaspiro[2.5]octan-6-yl)-2- (trifluoromethyl)nicotinonitrile |
| 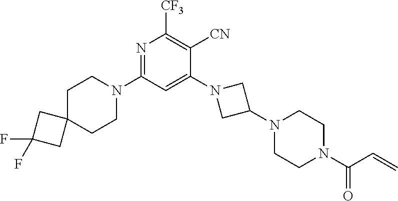 [SMILES: C=CC(=O)N1CCN(C2CN(c3cc(N4CCC5(CC4)CC(F)(F)C5)nc(C(F)(F)F)c3C#N)C2)CC1] | 4-(3-(4-acryloylpiperazin-1-yl)azetidin-1-yl)-6- (2,2-difluoro-7-azaspiro[3.5]nonan-7-yl)-2- (trifluoromethyl)nicotinonitrile |
| 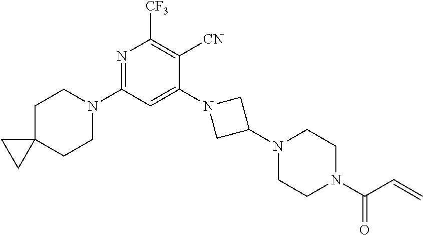 [SMILES: C=CC(=O)N1CCN(C2CN(c3cc(N4CCC5(CC4)CC5)nc(C(F)(F)F)c3C#N)C2)CC1] | 4-(3-(4-acryloylpiperazin-1-yl)azetidin-1-yl)-6-(6- azaspiro[2.5]octan-6-yl)-2- (trifluoromethyl)nicotinonitrile |
| 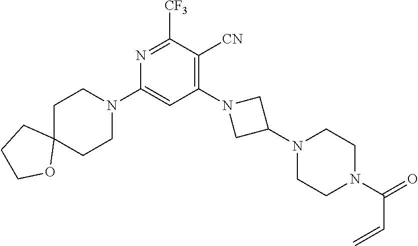 [SMILES: C=CC(=O)N1CCN(C2CN(c3cc(N4CCC5(CCCO5)CC4)nc(C(F)(F)F)c3C#N)C2)CC1] | 4-(3-(4-acryloylpiperazin-1-yl)azetidin-1-yl)-6-(1- oxa-8-azaspiro[4.5]decan-8-yl)-2- (trifluoromethyl)nicotinonitrile |
| 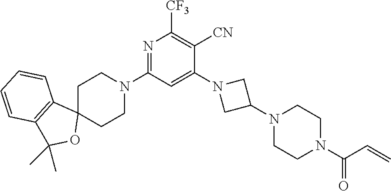 [SMILES: C=CC(=O)N1CCN(C2CN(c3cc(N4CCC5(CC4)OC(C)(C)c4ccccc45)nc(C(F)(F)F)c3C#N)C2)CC1] | 4-(3-(4-acryloylpiperazin-1-yl)azetidin-1-yl)-6- (3,3-dimethyl-3H-spiro[isobenzofuran-1,4′-piperi- din]-1′-yl)-2-(trifluoromethyl)nicotinonitrile |
| 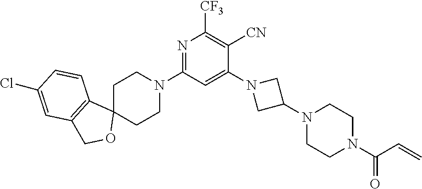 [SMILES: C=CC(=O)N1CCN(C2CN(c3cc(N4CCC5(CC4)OCc4cc(Cl)ccc45)nc(C(F)(F)F)c3C#N)C2)CC1] | 4-(3-(4-acryloylpiperazin-1-yl)azetidin-1-yl)-6-(5- chloro-3H-spirofisobenzofuran-1,4′-piperidin]-1′- yl)-2-(trifluoromethyl)nicotinonitrile |
| 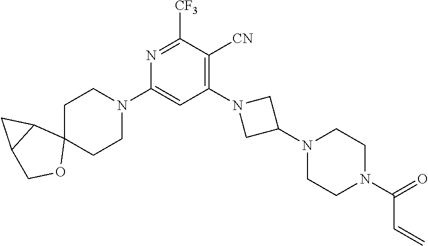 [SMILES: C=CC(=O)N1CCN(C2CN(c3cc(N4CCC5(CC4)OCC4CC45)nc(C(F)(F)F)c3C#N)C2)CC1] | 4-(3-(4-acryloylpiperazin-1-ylazetidin-1-yl)-6-(3- oxaspiro[bicyclo[3.1.0]hexane-2,4′-piperidin]-1′- yl)-2-(trifluoromethyl)nicotinonitrile |
| 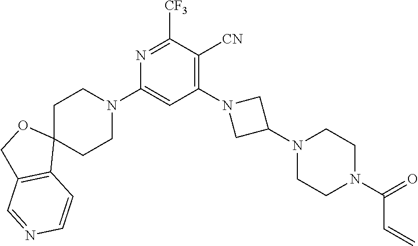 [SMILES: C=CC(=O)N1CCN(C2CN(c3cc(N4CCC5(CC4)OCc4cnccc45)nc(C(F)(F)F)c3C#N)C2)CC1] | 4-(3-(4-acryloylpiperazin-1-yl)azetidin-1-yl)-6- (3H-spiro[furo[3,4-c]pyridine-1,4′-piperidin]-1′- y])-2-(trifluoromethyl)nicotinonitrile |
| 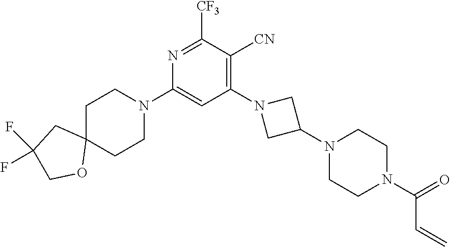 [SMILES: C=CC(=O)N1CCN(C2CN(c3cc(N4CCC5(CC4)CC(F)(F)CO5)nc(C(F)(F)F)c3C#N)C2)CC1] | 4-(3-(4-acryloylpiperazin-1-yl)azetidin-1-yl)-6- (3,3-difluoro-1-oxa-8-azaspiro[4.5]decan-8-yl)-2- (trifluoromethyl)nicotinonitrile |
| 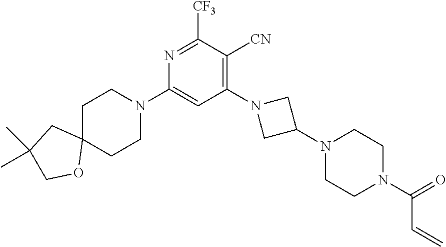 [SMILES: C=CC(=O)N1CCN(C2CN(c3cc(N4CCC5(CC4)CC(C)(C)CO5)nc(C(F)(F)F)c3C#N)C2)CC1] | 4-(3-(4-acryloylpiperazin-1-yl)azetidin-1-yl)-6- (3,3-dimethyl-1-oxa-8-azaspiro[4.5]decan-8-yl)-2- (trifluoromethyl)nicotinonitrile |
| 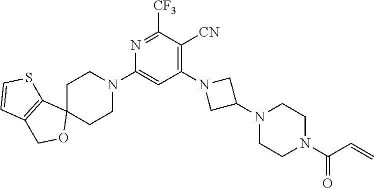 [SMILES: C=CC(=O)N1CCN(C2CN(c3cc(N4CCC5(CC4)OCc4ccsc45)nc(C(F)(F)F)c3C#N)C2)CC1] | 4-(3-(4-Acryloylpiperazin-1-yl)azetidin-1-yl)-6- (4H-spiro[furo[3,4-d]thiazole-6,4′-piperidin]-1′- yl)-2-(trifluoromethyl)nicotinonitrile |
| 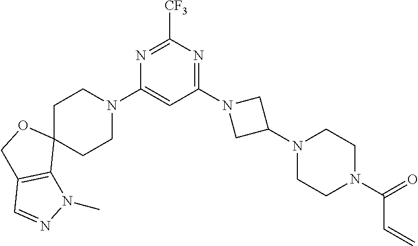 [SMILES: C=CC(=O)N1CCN(C2CN(c3cc(N4CCC5(CC4)OCc4cnn(C)c45)nc(C(F)(F)F)n3)C2)CC1] | 1-(4-(1-(6-(1-methyl-1,4-dihydrospiro[furo[3,4- c]pyrazole-6,4′-piperidin]-1′-yl)-2- (trifluoromethyl)pyrimidin-4-yl)azetidin-3- yl)piperazin-1-yl)prop-2-en-1-one |
| 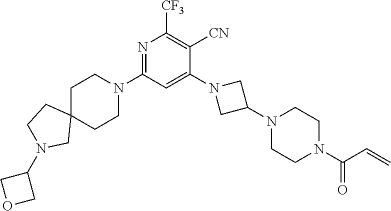 [SMILES: C=CC(=O)N1CCN(C2CN(c3cc(N4CCC5(CC4)CCN(C4COC4)C5)nc(C(F)(F)F)c3C#N)C2)CC1] | 4-(3-(4-acryloylpiperazin-1-yl)azetidin-1-yl)-6-(2- (oxetan-3-yl)-2,8-diazaspiro[4.5]decan-8-yl)-2- (trifluoromethyl)nicotinonitrile |
| 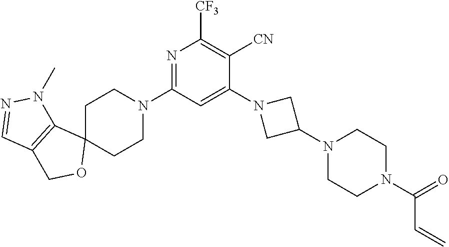 [SMILES: C=CC(=O)N1CCN(C2CN(c3cc(N4CCC5(CC4)OCc4cnn(C)c45)nc(C(F)(F)F)c3C#N)C2)CC1] | 4-(3-(4-acryloylpiperazin-1-yl)azetidin-1-yl)-6-(1- methyl-1,4-dihydrospiro[furo[3,4-c]pyrazole-6,4′- piperidin]-1′-yl)-2-(trifluoromethyl)nicotinonitrile |
| 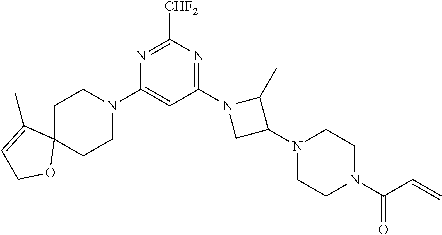 [SMILES: C=CC(=O)N1CCN(C2CN(c3cc(N4CCC5(CC4)OCC=C5C)nc(C(F)F)n3)C2C)CC1] | 1-(4-(1-(2-(difluoromethyl)-6-(4-methyl1-oxa-8- azaspiro[4.5]dec3-en-8-yl)pyrimidin-4-yl)-2- methylazetidin-3-yl)piperazin-1-yl)prop2-en-1- one |
| 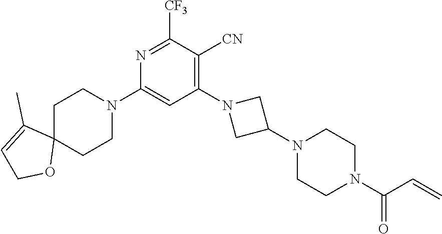 [SMILES: C=CC(=O)N1CCN(C2CN(c3cc(N4CCC5(CC4)OCC=C5C)nc(C(F)(F)F)c3C#N)C2)CC1] | 4-(3-(4-acryloylpiperazin-1-yl)azetidin-1-yl)-6-(4- methyl-1-oxa-8-azaspiro[4.5]dec-3-en-8-yl)-2- (trifluoromethyl)nicotinonitrile |
| 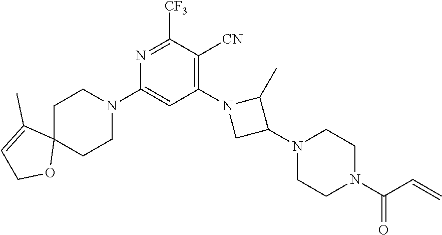 [SMILES: C=CC(=O)N1CCN(C2CN(c3cc(N4CCC5(CC4)OCC=C5C)nc(C(F)(F)F)c3C#N)C2C)CC1] | 4-(3-(4-acryloylpiperazin-1-yl)-2-methylazetidin- 1-yl)-6-(4-methyl-1-oxa-8-azaspiro[4.5]dec-3- en-8-yl)-2-(trifluoromethyl)nicotinonitrile |
| 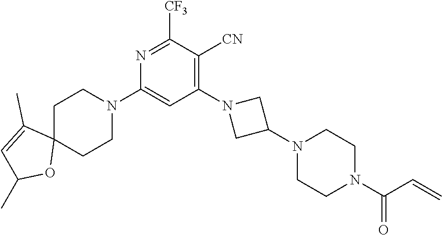 [SMILES: C=CC(=O)N1CCN(C2CN(c3cc(N4CCC5(CC4)OC(C)C=C5C)nc(C(F)(F)F)c3C#N)C2)CC1] | 4-(3-(4-acryloylpiperazin-1-yl)azetidin-1-yl)-6- (2,4-dimethyl-1-oxa-8-azaspiro[4.5]dec-3-en-8- yl)-2-(trifluoromethyl)nicotinonitrile |
| 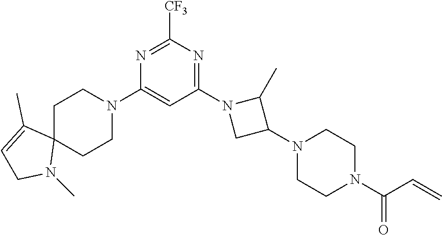 [SMILES: C=CC(=O)N1CCN(C2CN(c3cc(N4CCC5(CC4)C(C)=CCN5C)nc(C(F)(F)F)n3)C2C)CC1] | 1-(4-(1-(6-(1,4-dimethyl-1,8-diazaspiro[4.5]dec-3- en-8-yl)-2-(trifluoromethyl)pyrimidin-4-yl)-2- methylazetidin-3-yl)piperazin-1-yl)prop-2-enl-one |
| 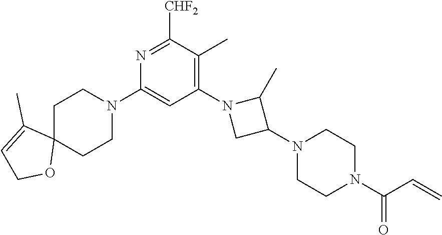 [SMILES: C=CC(=O)N1CCN(C2CN(c3cc(N4CCC5(CC4)OCC=C5C)nc(C(F)F)c3C)C2C)CC1] | 1-(4-(1-(2-(difluoromethyl)-3-methyl-6-(4-methyl- 1-oxa-8-azaspiro[4.5]dec-3-en-8-yl)pyridin-4-yl)- 2-methylazetidin-3-yl)piperazin-1-yl)prop-2- en-1-one |
| 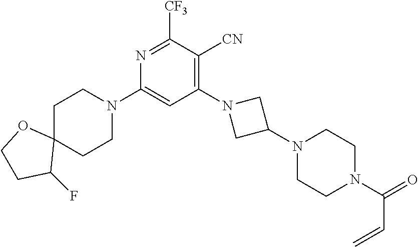 [SMILES: C=CC(=O)N1CCN(C2CN(c3cc(N4CCC5(CC4)OCCC5F)nc(C(F)(F)F)c3C#N)C2)CC1] | 4-(3-(4-acryloylpiperazin-1-yl)azetidin-1-yl)-6-(4- fluoro-1-oxa-8-azaspiro[4.5]decan-8-yl)-2- (trifluoromethyl)nicotinonitrile |
| 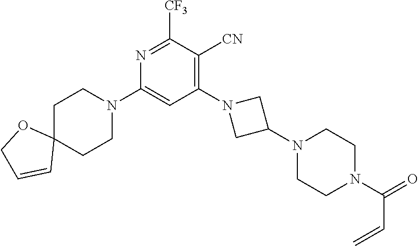 [SMILES: C=CC(=O)N1CCN(C2CN(c3cc(N4CCC5(C=CCO5)CC4)nc(C(F)(F)F)c3C#N)C2)CC1] | 4-(3-(4-acryloylpiperazin-1-y])azetidin-1-yl)-6-(1- oxa-8-azaspiro[4.5]dec-3-en-8-yl)-2- (trifluoromethyl)nicotinonitrile |
| 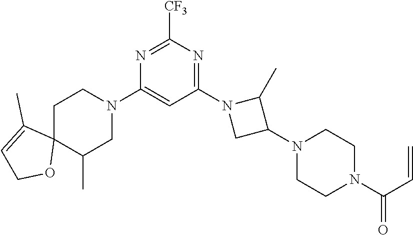 [SMILES: C=CC(=O)N1CCN(C2CN(c3cc(N4CCC5(OCC=C5C)C(C)C4)nc(C(F)(F)F)n3)C2C)CC1] | 1-(4-(1-(6-(4,6-dimethyl-1-oxa-8-azaspiro[4.5] dec-3-en-8-yl)-2-(trifluoromethyl)pyrimidin-4- yl)-2-methylazetidin-3-yl)piperazin-1-yl)prop-2- en-1-one |
| 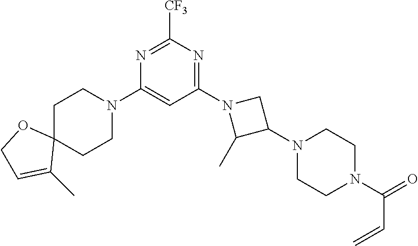 [SMILES: C=CC(=O)N1CCN(C2CN(c3cc(N4CCC5(CC4)OCC=C5C)nc(C(F)(F)F)n3)C2C)CC1] | 1-(4-(2-methy ]-1-(6-(4-methyl-1-oxa-8- azaspiro[4.5]dec-3-en-8-yl)-2- (trifluoromethyl)pyrimidin-4-yl)azetidin-3- yl)piperazin-1-yl)prop-2-en-1-one |
| 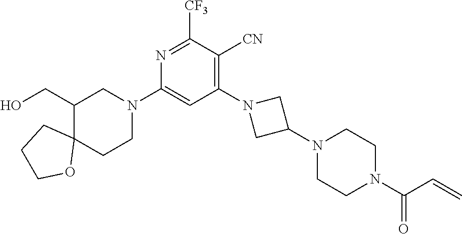 [SMILES: C=CC(=O)N1CCN(C2CN(c3cc(N4CCC5(CCCO5)C(CO)C4)nc(C(F)(F)F)c3C#N)C2)CC1] | 4-(3-(4-acryloylpiperazin-1-yl)azetidin-1-yl)-6-(6- (hydroxymethyl)-1-oxa-8-azaspiro[4.5]decan-8- yl)-2-(trifluoromethyl)nicotinonitrile |
| 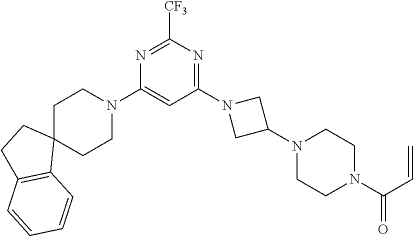 [SMILES: C=CC(=O)N1CCN(C2CN(c3cc(N4CCC5(CCc6ccccc65)CC4)nc(C(F)(F)F)n3)C2)CC1] | 1-(4-(1-(6-(2,3-dihydrospiro[indene-1,4′- piperidin]-1′-yl)-2-(trifluoromethyl)pyrimidin-4- yl)azetidin-3-yl)piperazin-1-yl)prop-2-en-1-one |
| 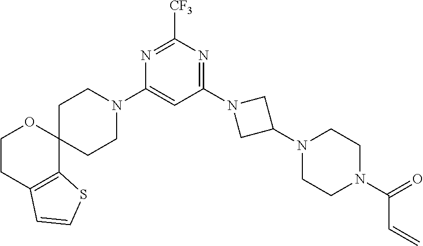 [SMILES: C=CC(=O)N1CCN(C2CN(c3cc(N4CCC5(CC4)OCCc4ccsc45)nc(C(F)(F)F)n3)C2)CC1] | 1-(4-(1-(6-(4′,5′-dihydrospiro[piperidine-4.7′- thieno[2,3-c]pyran]-1-yl)-2- (trifluoromethyl)pyrimidin-4-yl)azetidin-3- yl)piperazin-1-yl)prop-2-en-1-one |
| 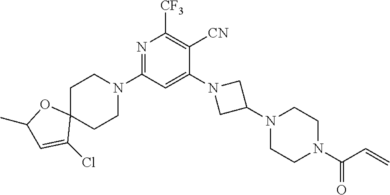 [SMILES: C=CC(=O)N1CCN(C2CN(c3cc(N4CCC5(CC4)OC(C)C=C5Cl)nc(C(F)(F)F)c3C#N)C2)CC1] | 4-(3-(4-acryloylpiperazin-1-yl)azetidin-1-yl)-6-(4- chloro-2-methyl-1-oxa-8-azaspiro[4.5]dec-3-en-8- yl)-2-(trifluoromethyl)nicotinonitrile |
| 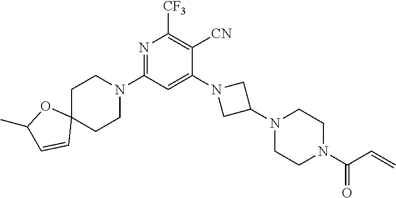 [SMILES: C=CC(=O)N1CCN(C2CN(c3cc(N4CCC5(C=CC(C)O5)CC4)nc(C(F)(F)F)c3C#N)C2)CC1] | 4-(3-(4-acryloylpiperazin-1-yl)azetidin-1-yl)-6-(2- methyl-1-oxa-8-azaspiro[4.5]dec-3-en-8-yl)-2- (trifluoromethyl)nicotinonitrile |
| 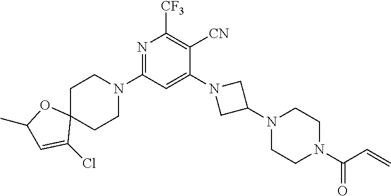 [SMILES: C=CC(=O)N1CCN(C2CN(c3cc(N4CCC5(CC4)OC(C)C=C5Cl)nc(C(F)(F)F)c3C#N)C2)CC1] | 4-(3-(4-acryloylpiperazin-1-yl)azetidin-1-yl)-6-(4- chloro-2-methyl-1-oxa-8-azaspiro[4.5]dec-3-en-8- yl)-2-(trifluoromethyl)nicotinonitrile |
| 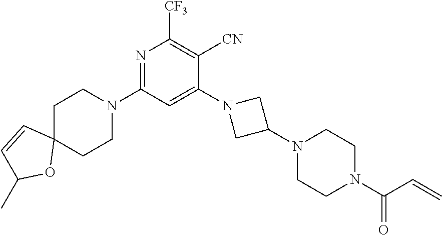 [SMILES: C=CC(=O)N1CCN(C2CN(c3cc(N4CCC5(C=CC(C)O5)CC4)nc(C(F)(F)F)c3C#N)C2)CC1] | 4-(3-(4-acryloylpiperazin-1-yl)azetidin-1-yl)-6-(2- methyl-1-oxa-8-azaspiro[4.5]dec-3-en-8-yl)-2- (trifluoromethyl)nicotinonitrile |
| 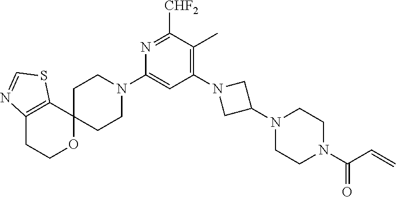 [SMILES: C=CC(=O)N1CCN(C2CN(c3cc(N4CCC5(CC4)OCCc4ncsc45)nc(C(F)F)c3C)C2)CC1] | 1-(4-(1-(2-(difluoromethyl)-6-(6′,7′- dihydrospiro[piperidine-4.4′-pyrano[4,3-d]thia- zol]-1-yl)-3-methylpyridin-4-yl)azetidin-3- yl)piperazin-1-yl)prop-2-en-1-one |
| 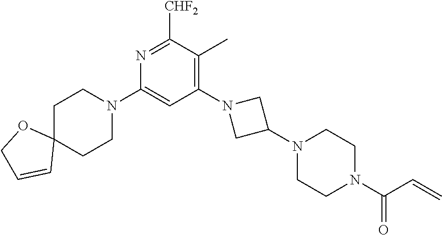 [SMILES: C=CC(=O)N1CCN(C2CN(c3cc(N4CCC5(C=CCO5)CC4)nc(C(F)F)c3C)C2)CC1] | 1-(4-(1-(2-(difluoromethyl)-3-methyl-6-(1-oxa-8- azaspiro[4.5]dec-3-en-8-yl)pyridin-4-yl)azetidin- 3-yl)piperazin-1-yl)prop-2-en-1-one |
| 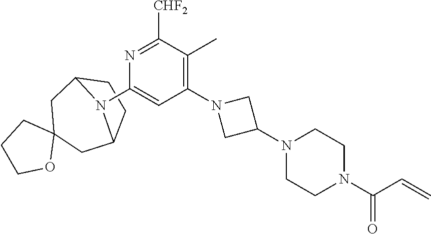 [SMILES: C=CC(=O)N1CCN(C2CN(c3cc(N4C5CCC4CC4(CCCO4)C5)nc(C(F)F)c3C)C2)CC1] | 1-(4-(1-(2-(difluoromethyl)-6-(dihydro-3′H-8- azaspiro[bicyclo[3.2.1]octane-3,2′-furan]-8-yl)-3- methylpyridin-4-yl)azetidin-3-yl)piperazin-1- yl)prop-2-en-1-one |
| 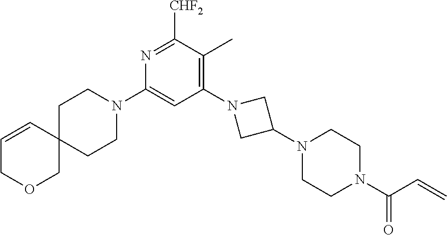 [SMILES: C=CC(=O)N1CCN(C2CN(c3cc(N4CCC5(C=CCOC5)CC4)nc(C(F)F)c3C)C2)CC1] | 1-(4-(1-(2-(difluoromethyl)-3-methyl-6-(2-oxa-9- azaspiro[5.5]undec-4-en-9-yl)pyridin-4- yl)azetidin-3-yl)piperazin-1-yl)prop-2-en-1-one |
| 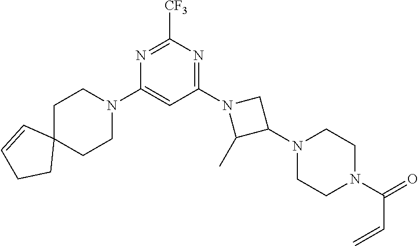 [SMILES: C=CC(=O)N1CCN(C2CN(c3cc(N4CCC5(C=CCC5)CC4)nc(C(F)(F)F)n3)C2C)CC1] | 1-(4-(1-(6-(8-azaspiro[4.5]dec-1-en-8-yl)-2- (trifluoromethyl)pyrimidin-4-yl)-2-methyl- azetidin-3-yl)piperazin-1-yl)prop-2-en-1-one |
| 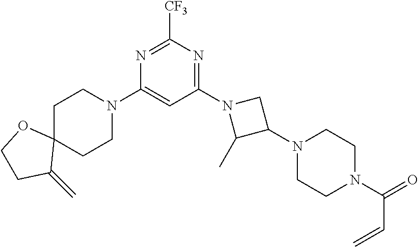 [SMILES: C=CC(=O)N1CCN(C2CN(c3cc(N4CCC5(CC4)OCCC5=C)nc(C(F)(F)F)n3)C2C)CC1] | 1-(4-((2-Methyl-1-(6-(4-methylene-1-oxa-8- azaspiro[4.5]decan8-yl)-2- (trifluoromethyl)pyrimidin-4-yl)azetidin-3- yl)piperazin-1-yl)prop-2-en-1-one |
| 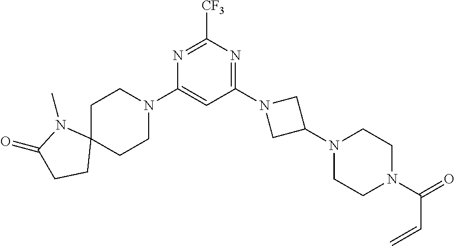 [SMILES: C=CC(=O)N1CCN(C2CN(c3cc(N4CCC5(CCC(=O)N5C)CC4)nc(C(F)(F)F)n3)C2)CC1] | 4-(3-(4-acryloylpiperazin-1-yl)azetidin-1-yl)-6- (1-methyl-2-oxo-1,8-diazaspiro[4.5]decan-8-yl)- 2-(trifluoromethyl)nicotinonitrile |
| 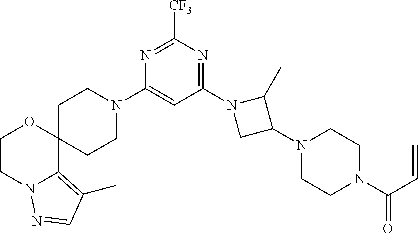 [SMILES: C=CC(=O)N1CCN(C2CN(c3cc(N4CCC5(CC4)OCCn4ncc(C)c45)nc(C(F)(F)F)n3)C2C)CC1] | 1-(4-(2-Methyl-1-(6-(3′-methyl-6′,7′- dihydrospiro [piperidine-4,4′-pyrazolo[5,1- c][1,4]oxazin]-1-yl)-2-(trifluoromethyl)pyrimidin- 4-yl)azetidin-3-yl)piperazin-1-yl)prop-2-en-1-one |
| 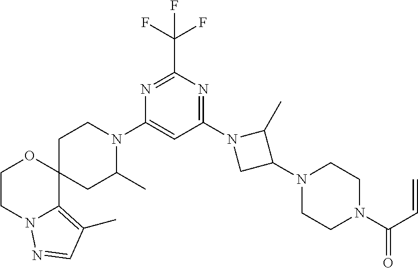 [SMILES: C=CC(=O)N1CCN(C2CN(c3cc(N4CCC5(CC4C)OCCn4ncc(C)c45)nc(C(F)(F)F)n3)C2C)CC1] | 1-(4-(1-(6-(2,3′-dimethyl-6′,7′-dihydro-1H- spiro[piperidine-4.4′-pyrazolo[5,1-c][1,4]oxazin]- 1-yl)-2-(trifluoromethyl)-4-pyrimidinyl)- 2-methyl-3-azetidinyl)-1-piperaziny1)-2- propen-1-one |
| 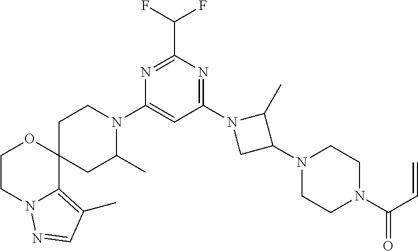 [SMILES: C=CC(=O)N1CCN(C2CN(c3cc(N4CCC5(CC4C)OCCn4ncc(C)c45)nc(C(F)F)n3)C2C)CC1] | 1-(4-(1-(2-(difluoromethyl)-6-(2,3′-dimethyl- 6′,7′-dihydro-1H-spiro[piperidine-4,4′-pyrazolo [5,1-c][1,4]oxazin]-1-yl)-4-pyrimidinyl)-2- methyl-3-azetidinyl)-1-piperazinyl)-2-propen- 1-one |
| 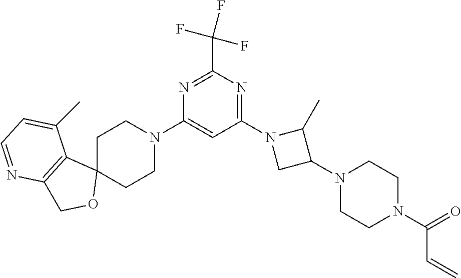 [SMILES: C=CC(=O)N1CCN(C2CN(c3cc(N4CCC5(CC4)OCc4nccc(C)c45)nc(C(F)(F)F)n3)C2C)CC1] | 1-(4-(2-methyl-1-(6-(4-methyl-1′H,7H- spiro[furo[3,4-b]pyridine-5,4′-piperidin]-1′-yl)-2- (trifluoromethyl)-4-pyrimidinyl)-3-azetidinyl)-1- piperazinyl)-2-propen-1-one |
| 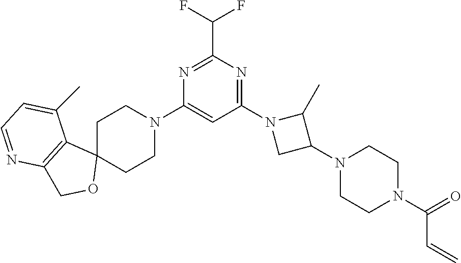 [SMILES: C=CC(=O)N1CCN(C2CN(c3cc(N4CCC5(CC4)OCc4nccc(C)c45)nc(C(F)F)n3)C2C)CC1] | 1-(4-(1-(2-(difluoromethyl)-6-(4-methyl-7H- spiro[furo[3.4-b]pyridine-5,4′-piperidin]-1′- yl)pyrimidin-4-yl)-2-methylazetidin-3- yl)piperazin-1-yl)prop-2-en-1-one |
| 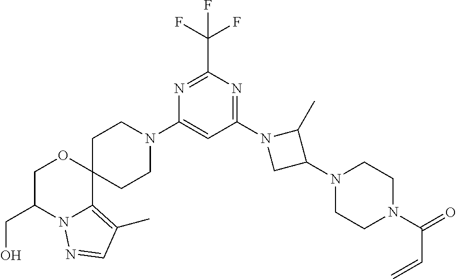 [SMILES: C=CC(=O)N1CCN(C2CN(c3cc(N4CCC5(CC4)OCC(CO)n4ncc(C)c45)nc(C(F)(F)F)n3)C2C)CC1] | 1-(4-(1-(6-(7′-(hydroxymethyl)-3′-methyl-6′,7′- dihydrospiro[piperidine-4,4′-pyrazolo[5,1-c] [1,4]oxazin]-1-yl)-2-(trifluoromethyl)pyrimidin- yl4-)-2-methylazetidin-3-yl)piperazin-1- yl)prop-2-en-1-one |
| 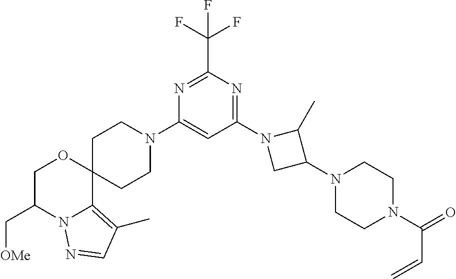 [SMILES: C=CC(=O)N1CCN(C2CN(c3cc(N4CCC5(CC4)OCC(COC)n4ncc(C)c45)nc(C(F)(F)F)n3)C2C)CC1] | 1-(4-(1-(6-(7′-(methoxymethyl)-3′-methyl-6′,7′- dihydrospiro[piperidine-4,4′-pyrazolo[5,1- c][1,4]oxazin]-1-yl)-2-(trifluoromethyl)pyrimi- din-4-yl)-2-methylazetidin-3-yl)piperazin-1- yl)prop-2-en-1-one |
| 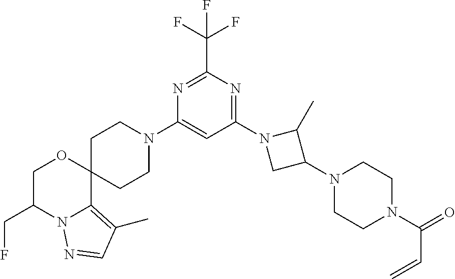 [SMILES: C=CC(=O)N1CCN(C2CN(c3cc(N4CCC5(CC4)OCC(CF)n4ncc(C)c45)nc(C(F)(F)F)n3)C2C)CC1] | 1-(4-(1-(6-(7′-(fluoromethyl)-3′-methyl-6′,7′- dihydrospiro[piperidine-4,4′-pyrazolo[5,1- c][1.4]oxazin]-1-yl)-2-(trifluoromethyl)pyrimi- din-4-yl)-2-methylazetidin-3-yl)piperazin- 11-yl)prop-2-en--one |
| 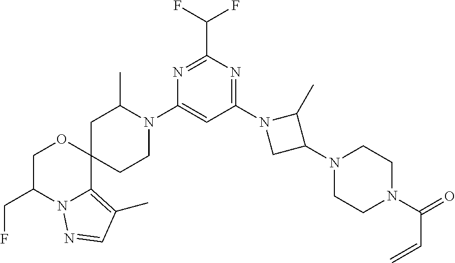 [SMILES: C=CC(=O)N1CCN(C2CN(c3cc(N4CCC5(CC4C)OCC(CF)n4ncc(C)c45)nc(C(F)F)n3)C2C)CC1] | 1-(4-(1-(2-(difluoromethyl)-6-(7-(fluoromethyl)- 2,3′-dimethyl-6′,7′-dihydrospiro[piperidine-4,4′- pyrazolo[5,1-c][1,4]oxazin]-1-yl)pyrimidin-4-yl)- 2-methylazetidin-3-yl)piperazin-1-yl)prop-2-en-1- one |
| 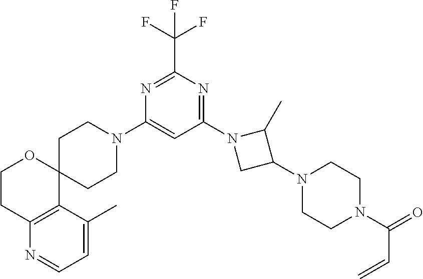 [SMILES: C=CC(=O)N1CCN(C2CN(c3cc(N4CCC5(CC4)OCCc4nccc(C)c45)nc(C(F)(F)F)n3)C2C)CC1] | 1-(4-(2-methyl-1-(6-(4′-methy1-7′,8′- dihydrospiro[piperidine-4.5′-pyrano[4,3- b]pyridin]-1-yl)-2-(trifluoromethyl)pyrimidin-4- yl)azetidin-3-yl)piperazin-1-yl)prop-2-en-1-one |
| 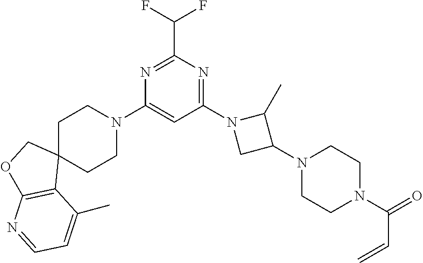 [SMILES: C=CC(=O)N1CCN(C2CN(c3cc(N4CCC5(CC4)COc4nccc(C)c45)nc(C(F)F)n3)C2C)CC1] | 1-(4-(1-(2-(difluoromethyl)-6-(4-methyl-2H- spiro[furo[2,3-b]pyridine-3,4′-piperidin]-1′- yl)pyrimidin-4-yl)-2-methylazetidin- 3-yl)piperazin-1-yl)prop-2-en-1-one |
| 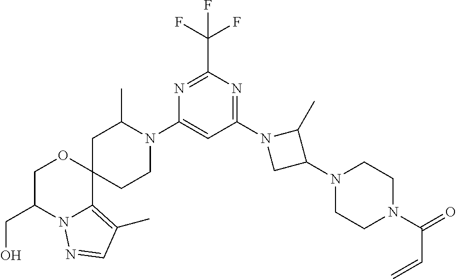 [SMILES: C=CC(=O)N1CCN(C2CN(c3cc(N4CCC5(CC4C)OCC(CO)n4ncc(C)c45)nc(C(F)(F)F)n3)C2C)CC1] | 1-(4-(1-(6-(7′-(hydroxymethyl)-2,3′-dimethyl- 6′,7′-dihydrospiro [piperidine-4,4′-pyrazolo[5,1- c][1,4]oxazin]-1-yl)-2-(trifluoromethyl)pyrimi- din-4-yl)-2-methylazetidin-3-yl)piperazin- 1-yl)prop-2-en-1-one |
| 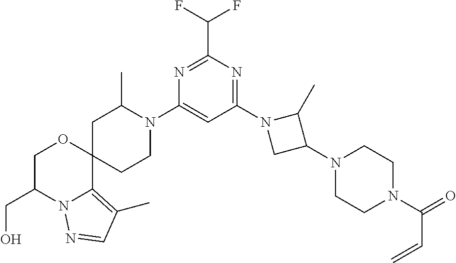 [SMILES: C=CC(=O)N1CCN(C2CN(c3cc(N4CCC5(CC4C)OCC(CO)n4ncc(C)c45)nc(C(F)F)n3)C2C)CC1] | 1-(4-(1-(2-(difluoromethy1)-6-(7′-(hydroxy- methyl)-2,3′-dimethyl-6′,7′-dihydrospiro[piperi- dine-4,4′-pyrazolo[5,1-c][1,4]oxazin]-1- yl)pyrimidin-4-yl)-2-methylazetidin-3- yl)piperazin-1-yl)prop-2-en-1-one |
| 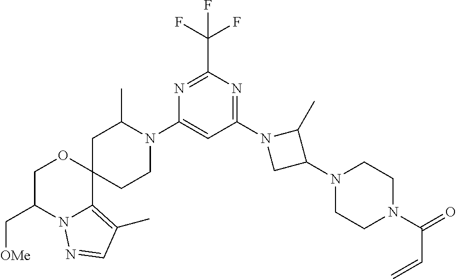 [SMILES: C=CC(=O)N1CCN(C2CN(c3cc(N4CCC5(CC4C)OCC(COC)n4ncc(C)c45)nc(C(F)(F)F)n3)C2C)CC1] | 1-(4-(1-(6-((7′-(methoxymethyl)-2,3′-dimethyl- 6′,7′-dihydrospiro[piperidine-4,4′-pyrazolo[5.1- c][1,4]oxazin]-1-yl)-2-(trifluoromethyl)pyrimi- din-4-yl)-2-methylazetidin-3-yl)piperazin- 1-yl)prop-2-en-1-one |
| 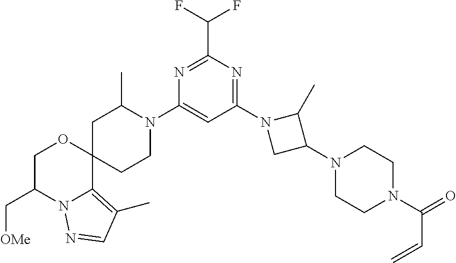 [SMILES: C=CC(=O)N1CCN(C2CN(c3cc(N4CCC5(CC4C)OCC(COC)n4ncc(C)c45)nc(C(F)F)n3)C2C)CC1] | 1-(4-(1-(2-(difluoromethyl)-6-(7′- (methoxymethyl)-2,3′-dimethyl- 6′,7′-dihydrospiro[piperidine-4,4′-py- razolo[5,1-c][1,4]oxazin]-1-yl)pyrimidin-4-yl)-2- methylazetidin-3-yl)piperazin-1-yl)prop-2-en-1- one |
| 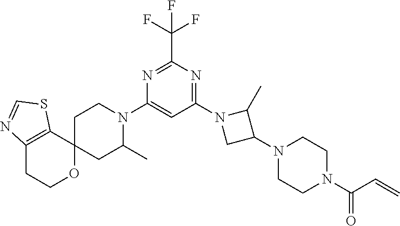 [SMILES: C=CC(=O)N1CCN(C2CN(c3cc(N4CCC5(CC4C)OCCc4ncsc45)nc(C(F)(F)F)n3)C2C)CC1] | 1-(4-(2-methy1-1-(6-(2-methyl-6′,7′- dihydrospiro[piperidine-4,4′-pyrano[4,3- d]thiazol]-1-yl)-2-(trifluoromethyl) pyrimidin-4-yl)azetidin-3- yl)piperazin-1-yl)prop-2-en-1-one |
| 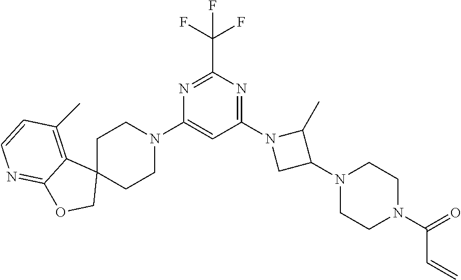 [SMILES: C=CC(=O)N1CCN(C2CN(c3cc(N4CCC5(CC4)COc4nccc(C)c45)nc(C(F)(F)F)n3)C2C)CC1] | 1-(4-(2-methyl-1-(6-(4-methyl-1′H-spiro[furo [2.3-b]pyridine-3,4′-piperidin]-1′-yl)- 2-(trifluoromethyl)-4-pyrimidinyl)-3- azetidinyl)-1-piperazinyl)-2-propen-1-one |
| 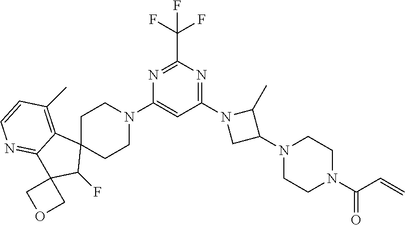 [SMILES: C=CC(=O)N1CCN(C2CN(c3cc(N4CCC5(CC4)c4c(C)ccnc4C4(COC4)C5F)nc(C(F)(F)F)n3)C2C)CC1] | 1-(4-(1-(6-(6′-fluoro-4′-methyl-1″H- dispirofoxetane-3.7′-cyclopenta[b]pyridine- 5′.4″-piperidin]-1″-yl)-2- (trifluoromethyl)-4-pyrimidinyl)-2-methyl- 3-azetidinyl)-1-piperazinyl)-2-propen-1-one |
| 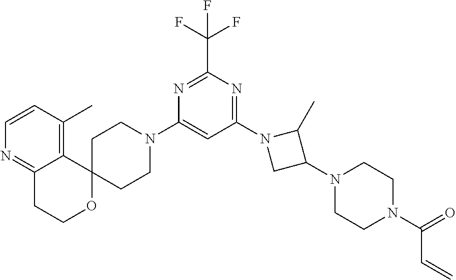 [SMILES: C=CC(=O)N1CCN(C2CN(c3cc(N4CCC5(CC4)OCCc4nccc(C)c45)nc(C(F)(F)F)n3)C2C)CC1] | 1-(4-(2-methyl-1-(6-(4′-methyl-7′,8′-dihydro-1H- spiro(piperidine-4,5′-pyrano[4,3-b]pyridin]-1-yl)- 2-(trifluoromethyl)-4-pyrimidinyl)-3-azetidinyl)- 1-piperazinyl)-2-propen-1-one |
| 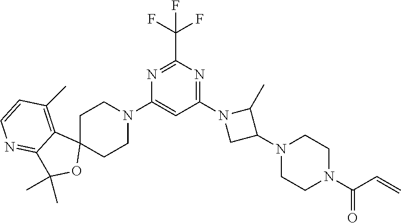 [SMILES: C=CC(=O)N1CCN(C2CN(c3cc(N4CCC5(CC4)OC(C)(C)c4nccc(C)c45)nc(C(F)(F)F)n3)C2C)CC1] | 1-(4-(2-methyl-1-(2-(trifluoromethyl)-6-(4,7,7- trimethyl-1′H,7H-spiro[furo[3,4-b]pyridine-5,4′- piperidin]-1′-yl)-4-pyrimidinyl)-3-azetidinyl)-1- piperazinyl)-2-propen-1-one |
| 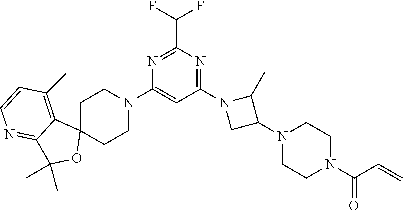 [SMILES: C=CC(=O)N1CCN(C2CN(c3cc(N4CCC5(CC4)OC(C)(C)c4nccc(C)c45)nc(C(F)F)n3)C2C)CC1] | 1-(4-(1-(2-(difluoromethyl)-6-(4,7,7-trimethyl- 1′H,7H-spiro[furo[3,4-b]pyridine-5,4′-piperidin]- 1′-yl)-4-pyrimidinyl)-2-methyl-3-azetidinyl)-1- piperazinyl)-2-propen-1-one |
| 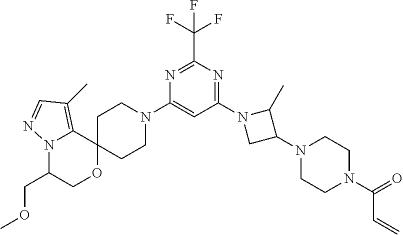 [SMILES: C=CC(=O)N1CCN(C2CN(c3cc(N4CCC5(CC4)OCC(COC)n4ncc(C)c45)nc(C(F)(F)F)n3)C2C)CC1] | 1-(4-(1-(6-(7′-(methoxymethyl)-3′-methyl-6′,7- dihydro-1H-spiro[piperidine-4,4′-pyrazolo[5,1- c][1,4]oxazin]-1-yl)-2-(trifluoromethyl)-4- pyrimidinyl)-2-methyl-3-azetidinyl)-1- piperazinyl)-2-propen-1-one |
| 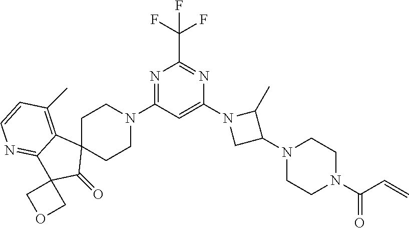 [SMILES: C=CC(=O)N1CCN(C2CN(c3cc(N4CCC5(CC4)C(=O)C4(COC4)c4nccc(C)c45)nc(C(F)(F)F)n3)C2C)CC1] | 4′-methyl-1″-(6-(2-methyl-3-(4-(2-propenoy])-1- piperazinyl)-1-azetidinyl)-2-(trifluoromethyl)-4- pyrimidinyl)dispiro[oxetane-3.7′- cyclopenta[b]pyridine-5′,4″-piperidin]-6′-one |
| 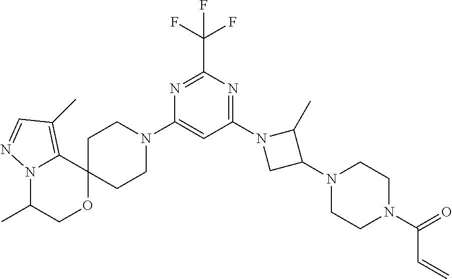 [SMILES: C=CC(=O)N1CCN(C2CN(c3cc(N4CCC5(CC4)OCC(C)n4ncc(C)c45)nc(C(F)(F)F)n3)C2C)CC1] | 1-(4-(1-(6-(3′,7′-dimethyl-6′,7′-dihydro-1H- spiro[piperidine-4,4′-pyrazolo[5,1-c]\|1,4]oxa- zin]-1-yl)-2-(trifluoromethyl)-4- pyrimidinyl)-2-methyl-3- azetidinyl)-1-piperazinyl)-2-propen-1-one |
| - 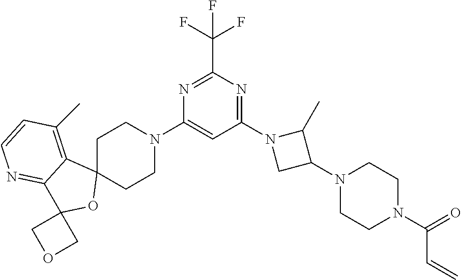 [SMILES: C=CC(=O)N1CCN(C2CN(c3cc(N4CCC5(CC4)OC4(COC4)c4nccc(C)c45)nc(C(F)(F)F)n3)C2C)CC1] | 1-(4-(2-methy1-1-(6-(4′-methyl-1″H- dispirofoxetane-3,7′-furo[3,4-b] pyridine-5′,4″-piperidin]-1″-yl)-2-(tri- fluoromethyl)-4-pyrimidinyl)-3-azetidinyl)-1- piperazinyl)-2-propen-1-one |
| 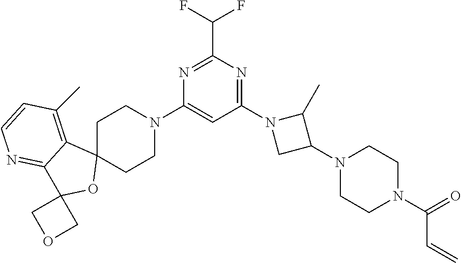 [SMILES: C=CC(=O)N1CCN(C2CN(c3cc(N4CCC5(CC4)OC4(COC4)c4nccc(C)c45)nc(C(F)F)n3)C2C)CC1] | 1-(4-(1-(2-(difluoromethy1)-6-(4′-methyl-1″H- dispiro[oxetane-3,7′-furo[3,4-b]pyridine-5′,4″- piperidin]-1″-yl)-4-pyrimidinyl)-2-methyl-3- azetidinyl)-1-piperazinyl)-2-propen-1-one |
| 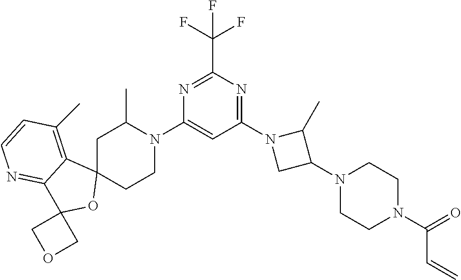 [SMILES: C=CC(=O)N1CCN(C2CN(c3cc(N4CCC5(CC4C)OC4(COC4)c4nccc(C)c45)nc(C(F)(F)F)n3)C2C)CC1] | 1-(4-(1-(6-(2″,4′-dimethyl-1″H-dispiro[oxetane- 3,7′-furo(3,4-b]pyridine-5′,4″-piperidin] -1″-yl)- 2-(trifluoromethyl)-4-pyrimidinyl)-2-methyl- 3-azetidinyl)-1-piperazinyl)-2-propen-1-one |

In some cases, the compound of Formula (I) or Formula (II) is selected from the list of compounds in Table A.

In some cases, the disclosure provides compounds of Formula (I) or Formula (II), or pharmaceutically acceptable salts of any of the foregoing, wherein Y′ is C₃₋₇cycloalkyl or C₄₋₇cycloalkenyl. Examples of such compounds include, but are not limited to:

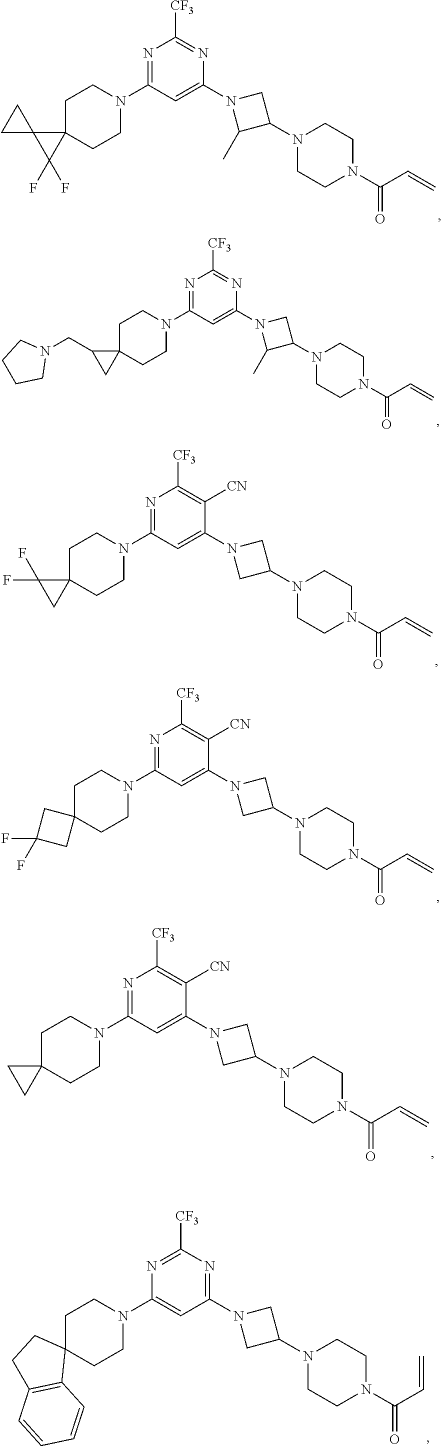 [SMILES: C=CC(=O)N1CCN(C2CN(c3cc(N4CCC5(CC4)C(F)(F)C54CC4)nc(C(F)(F)F)n3)C2C)CC1 ; C=CC(=O)N1CCN(C2CN(c3cc(N4CCC5(CC4)CC5CN4CCCC4)nc(C(F)(F)F)n3)C2C)CC1 ; C=CC(=O)N1CCN(C2CN(c3cc(N4CCC5(CC4)CC5(F)F)nc(C(F)(F)F)c3C#N)C2)CC1 ; C=CC(=O)N1CCN(C2CN(c3cc(N4CCC5(CC4)CC(F)(F)C5)nc(C(F)(F)F)c3C#N)C2)CC1 ; C=CC(=O)N1CCN(C2CN(c3cc(N4CCC5(CC4)CC5)nc(C(F)(F)F)c3C#N)C2)CC1 ; C=CC(=O)N1CCN(C2CN(c3cc(N4CCC5(CCc6ccccc65)CC4)nc(C(F)(F)F)n3)C2)CC1]

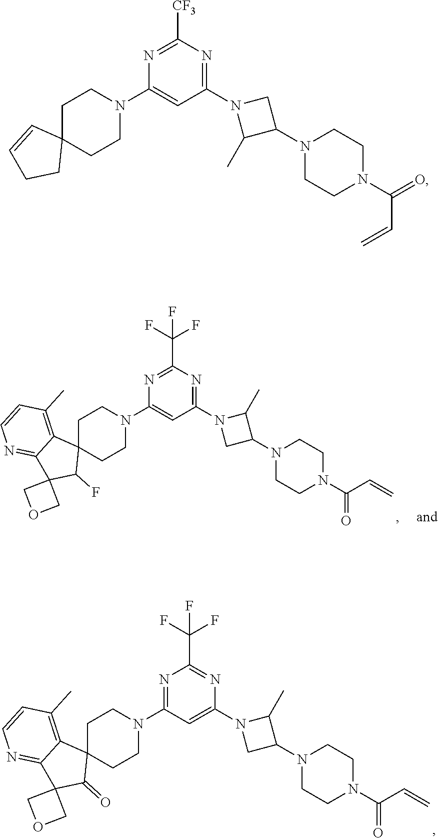 [SMILES: C=CC(=O)N1CCN(C2CN(c3cc(N4CCC5(C=CCC5)CC4)nc(C(F)(F)F)n3)C2C)CC1 ; C=CC(=O)N1CCN(C2CN(c3cc(N4CCC5(CC4)c4c(C)ccnc4C4(COC4)C5F)nc(C(F)(F)F)n3)C2C)CC1 ; C=CC(=O)N1CCN(C2CN(c3cc(N4CCC5(CC4)C(=O)C4(COC4)c4nccc(C)c45)nc(C(F)(F)F)n3)C2C)CC1]

and pharmaceutically acceptable salts thereof.

In some cases, the disclosure provides compounds of Formula (I) or (II), or pharmaceutically acceptable salts of any of the foregoing, wherein Y′ is oxetanyl or dioxanyl. Examples of such compounds include, but are not limited to:

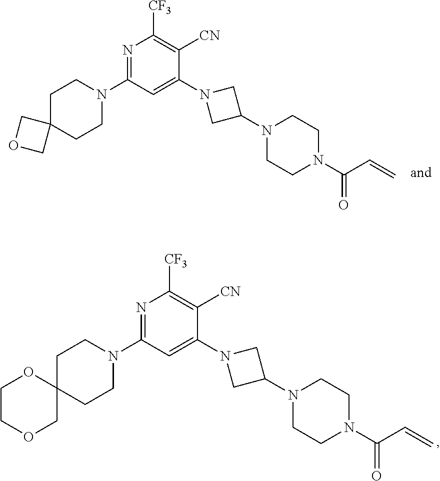 [SMILES: C=CC(=O)N1CCN(C2CN(c3cc(N4CCC5(CC4)COC5)nc(C(F)(F)F)c3C#N)C2)CC1 ; C=CC(=O)N1CCN(C2CN(c3cc(N4CCC5(CC4)COCCO5)nc(C(F)(F)F)c3C#N)C2)CC1]

and pharmaceutically acceptable salts thereof.

In some cases, the disclosure provides compounds of Formula (I) or Formula (II), or pharmaceutically acceptable salts of any of the foregoing, wherein Y′ is tetrahydrofuranyl and p is 0. Examples of such compounds include, but are not limited to:

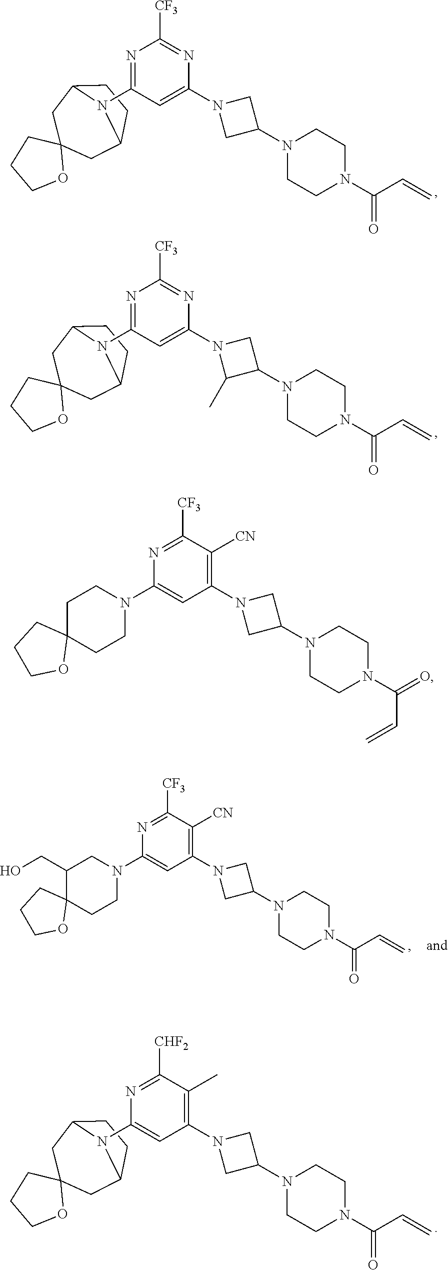 [SMILES: C=CC(=O)N1CCN(C2CN(c3cc(N4C5CCC4CC4(CCCO4)C5)nc(C(F)(F)F)n3)C2)CC1 ; C=CC(=O)N1CCN(C2CN(c3cc(N4C5CCC4CC4(CCCO4)C5)nc(C(F)(F)F)n3)C2C)CC1 ; C=CC(=O)N1CCN(C2CN(c3cc(N4CCC5(CCCO5)CC4)nc(C(F)(F)F)c3C#N)C2)CC1 ; C=CC(=O)N1CCN(C2CN(c3cc(N4CCC5(CCCO5)C(CO)C4)nc(C(F)(F)F)c3C#N)C2)CC1 ; C=CC(=O)N1CCN(C2CN(c3cc(N4C5CCC4CC4(CCCO4)C5)nc(C(F)F)c3C)C2)CC1]

and pharmaceutically acceptable salts of any of the foregoing.

In some cases, the disclosure provides compounds of Formula (I) or Formula (II), or pharmaceutically acceptable salts of any of the foregoing, wherein Y′ is tetrahydrofuranyl and each p is CH₂, oxo, halo, CN, C₁₋₃alkyl, C₁₋₃haloalkyl, C₂₋₃alkenyl, C₀₋₃alkyleneOH, C₁₋₃alkylene-C₁₋₃alkoxy, C(═O)OC₁₋₃alkyl, C₁₋₃alkylene-C₃₋₇cycloalkyl, C₀₋₄alkylene-heterocycloalkyl having 3-7 total ring atoms and 1, 2, or 3 heteroatoms selected from N, O, and S, C₀₋₄alkylene-C₆₋₁₀aryl, C₀₋₃alkylene-heteroaryl having 5-7 total ring atoms and 1, 2, or 3 heteroatoms selected from N, O, and S. Examples of such compounds include, but are not limited to:

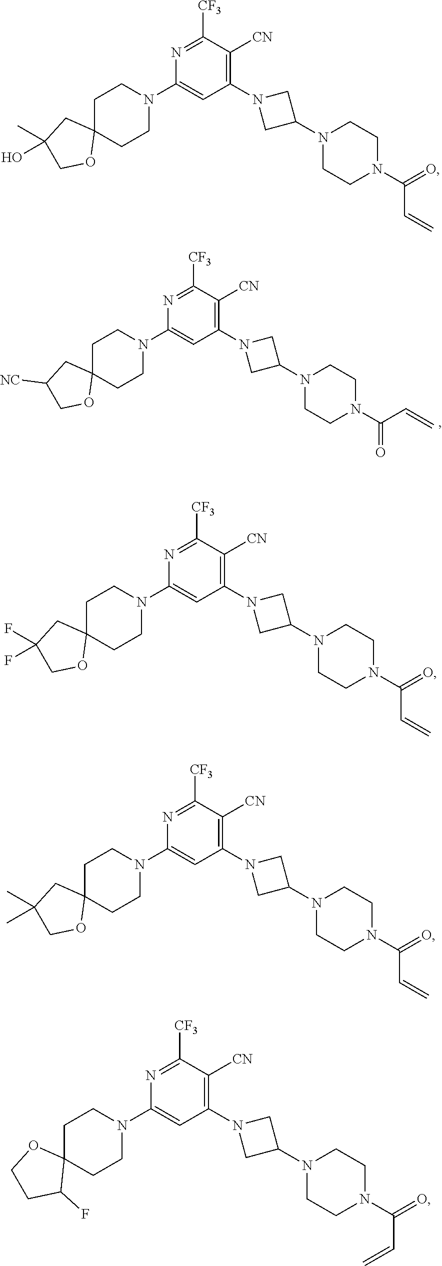

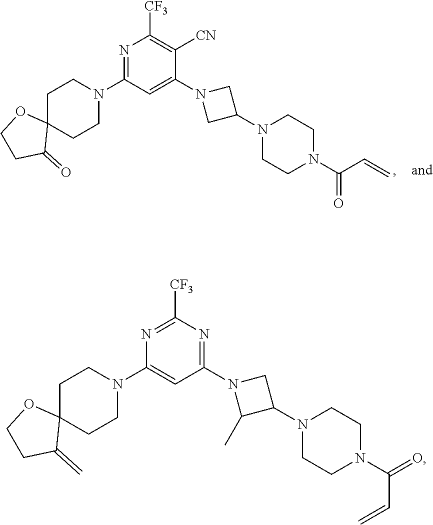 [SMILES: C=CC(=O)N1CCN(C2CN(c3cc(N4CCC5(CC4)OCCC5=O)nc(C(F)(F)F)c3C#N)C2)CC1 ; C=CC(=O)N1CCN(C2CN(c3cc(N4CCC5(CC4)OCCC5=C)nc(C(F)(F)F)n3)C2C)CC1]

and pharmaceutically acceptable salts of any of the foregoing.

In some cases, the disclosure provides compounds of Formula (I) or Formula (II), or pharmaceutically acceptable salts of any of the foregoing, wherein Y′ is tetrahydrofuranyl and two vicinal R⁷ together with the atoms to which they are attached form optionally substituted fused-C₃₋₇cycloalkyl or optionally substituted fused-C₆₋₁₀aryl. In some cases, the cycloalkyl and aryl is unsubstituted or substituted with 1-3 substituents, and each substituent independently is halo, CN, oxo, or C₁₋₃alkyl. Examples of such compounds include, but are not limited to:

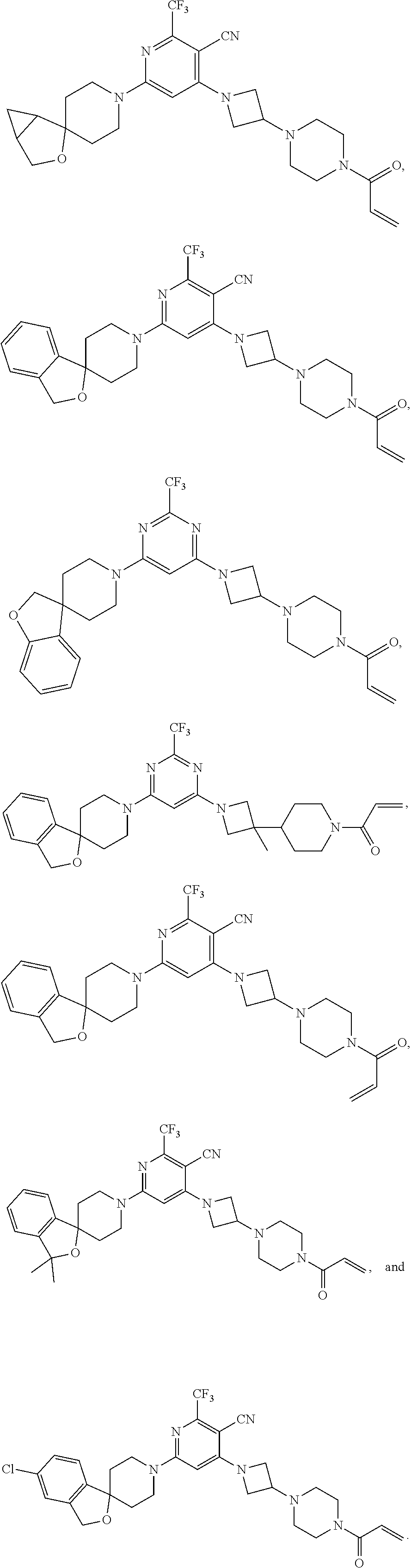

and pharmaceutically acceptable salts thereof.

In some cases, the disclosure provides compounds of Formula (I) or Formula (II), or pharmaceutically acceptable salts of any of the foregoing, wherein Y′ is tetrahydrofuranyl and two vicinal R⁷ together with the atoms to which they are attached form optionally substituted fused-heteroaryl having 5-7 total ring atoms and 1-3 heteroatoms independently selected from N, O, and S. In some cases, the heteroaryl is unsubstituted or substituted with 1-3 substituents, and each substituent independently is halo, CN, oxo, or C₁₋₃alkyl. Examples of such compounds include, but are not limited to:

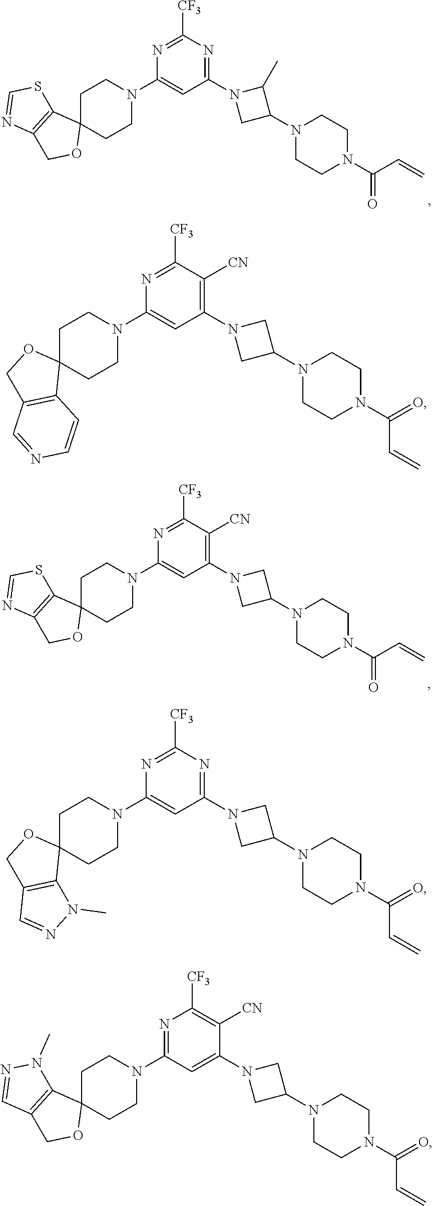 [SMILES: C=CC(=O)N1CCN(C2CN(c3cc(N4CCC5(CC4)OCc4ncsc45)nc(C(F)(F)F)n3)C2C)CC1 ; C=CC(=O)N1CCN(C2CN(c3cc(N4CCC5(CC4)OCc4cnccc45)nc(C(F)(F)F)c3C#N)C2)CC1 ; C=CC(=O)N1CCN(C2CN(c3cc(N4CCC5(CC4)OCc4ncsc45)nc(C(F)(F)F)c3C#N)C2)CC1 ; C=CC(=O)N1CCN(C2CN(c3cc(N4CCC5(CC4)OCc4cnn(C)c45)nc(C(F)(F)F)n3)C2)CC1 ; C=CC(=O)N1CCN(C2CN(c3cc(N4CCC5(CC4)OCc4cnn(C)c45)nc(C(F)(F)F)c3C#N)C2)CC1]

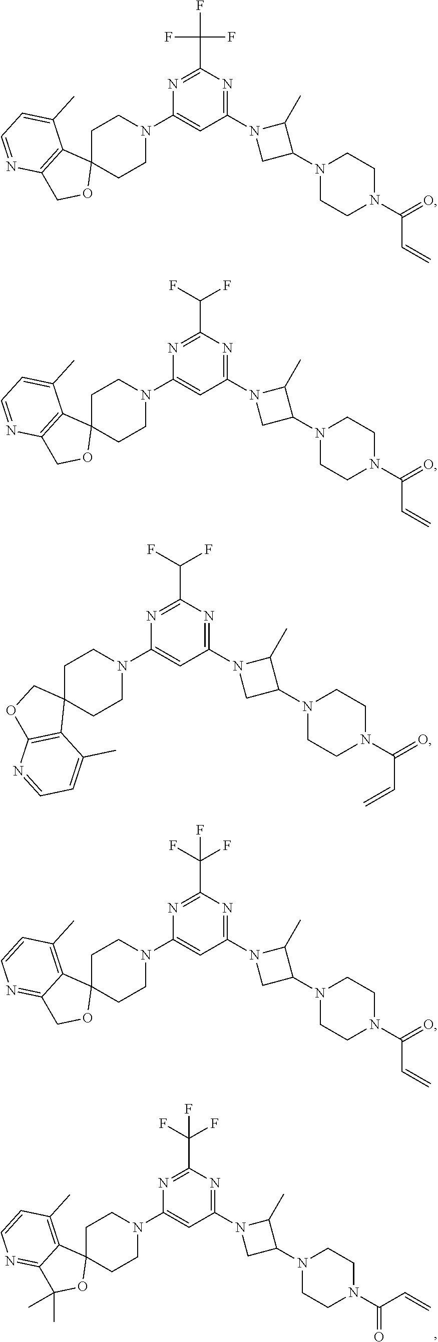 [SMILES: C=CC(=O)N1CCN(C2CN(c3cc(N4CCC5(CC4)OCc4nccc(C)c45)nc(C(F)(F)F)n3)C2C)CC1 ; C=CC(=O)N1CCN(C2CN(c3cc(N4CCC5(CC4)OCc4nccc(C)c45)nc(C(F)F)n3)C2C)CC1 ; C=CC(=O)N1CCN(C2CN(c3cc(N4CCC5(CC4)COc4nccc(C)c45)nc(C(F)F)n3)C2C)CC1 ; C=CC(=O)N1CCN(C2CN(c3cc(N4CCC5(CC4)OCc4nccc(C)c45)nc(C(F)(F)F)n3)C2C)CC1 ; C=CC(=O)N1CCN(C2CN(c3cc(N4CCC5(CC4)OC(C)(C)c4nccc(C)c45)nc(C(F)(F)F)n3)C2C)CC1]

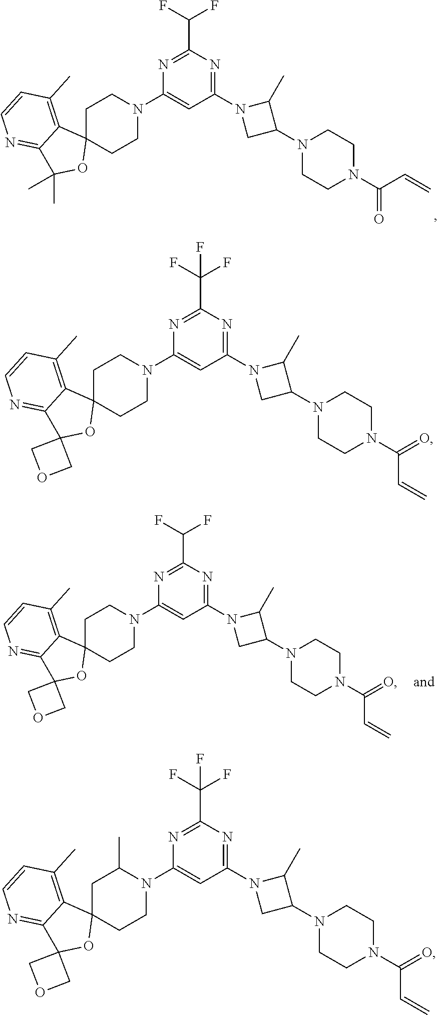 [SMILES: C=CC(=O)N1CCN(C2CN(c3cc(N4CCC5(CC4)OC(C)(C)c4nccc(C)c45)nc(C(F)F)n3)C2C)CC1 ; C=CC(=O)N1CCN(C2CN(c3cc(N4CCC5(CC4)OC4(COC4)c4nccc(C)c45)nc(C(F)(F)F)n3)C2C)CC1 ; C=CC(=O)N1CCN(C2CN(c3cc(N4CCC5(CC4)OC4(COC4)c4nccc(C)c45)nc(C(F)F)n3)C2C)CC1 ; C=CC(=O)N1CCN(C2CN(c3cc(N4CCC5(CC4C)OC4(COC4)c4nccc(C)c45)nc(C(F)(F)F)n3)C2C)CC1]

and pharmaceutically acceptable salts thereof.

In some cases, the disclosure provides compounds of Formula (I) or Formula (II), or pharmaceutically acceptable salts of any of the foregoing, wherein Y′ is tetrahydropyranyl and p is 0.

An example of such a compound is

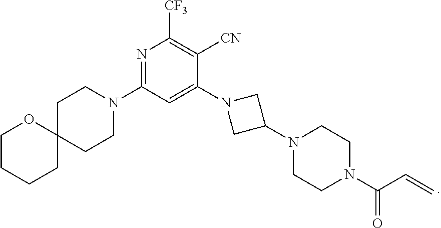 [SMILES: C=CC(=O)N1CCN(C2CN(c3cc(N4CCC5(CCCCO5)CC4)nc(C(F)(F)F)c3C#N)C2)CC1]

and pharmaceutically acceptable salts thereof.

In some cases, the disclosure provides compounds of Formula (I) or Formula (II), or pharmaceutically acceptable salts of any of the foregoing, wherein Y′ is tetrahydropyranyl and two vicinal R⁷, together with the atoms to which they are attached, form a fused thiazolyl ring, a fused thiophenyl ring, or a fused pyridyl ring. Examples of such compounds include, but are not limited to:

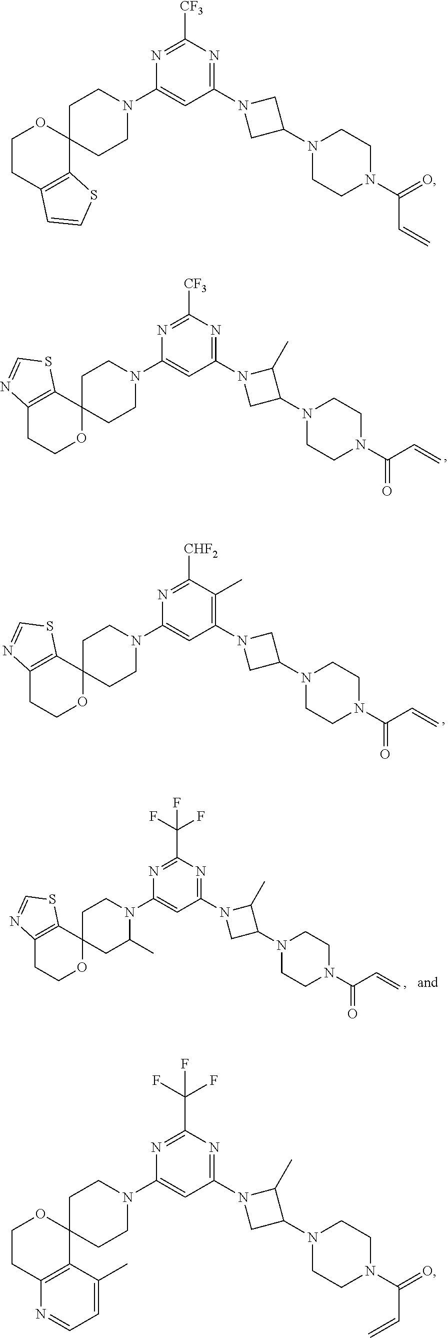 [SMILES: C=CC(=O)N1CCN(C2CN(c3cc(N4CCC5(CC4)OCCc4ccsc45)nc(C(F)(F)F)n3)C2)CC1 ; C=CC(=O)N1CCN(C2CN(c3cc(N4CCC5(CC4)OCCc4ncsc45)nc(C(F)(F)F)n3)C2C)CC1 ; C=CC(=O)N1CCN(C2CN(c3cc(N4CCC5(CC4)OCCc4ncsc45)nc(C(F)F)c3C)C2)CC1 ; C=CC(=O)N1CCN(C2CN(c3cc(N4CCC5(CC4C)OCCc4ncsc45)nc(C(F)(F)F)n3)C2C)CC1 ; C=CC(=O)N1CCN(C2CN(c3cc(N4CCC5(CC4)OCCc4nccc(C)c45)nc(C(F)(F)F)n3)C2C)CC1]

and pharmaceutically acceptable salts thereof.

In some cases, the disclosure provides compounds of Formula (I) or Formula (II), or pharmaceutically acceptable salts of any of the foregoing, wherein Y′ is morpholinyl; p is 2 or 3; and two vicinal R⁷ together with the atoms to which they are attached form optionally substituted fused-pyrazolyl. In some cases, the pyrazolyl is unsubstituted or substituted with 1-3 substituents, and each substituent independently is halo, CN, oxo, or Ct-alkyl. Examples of such compounds include, but are not limited to:

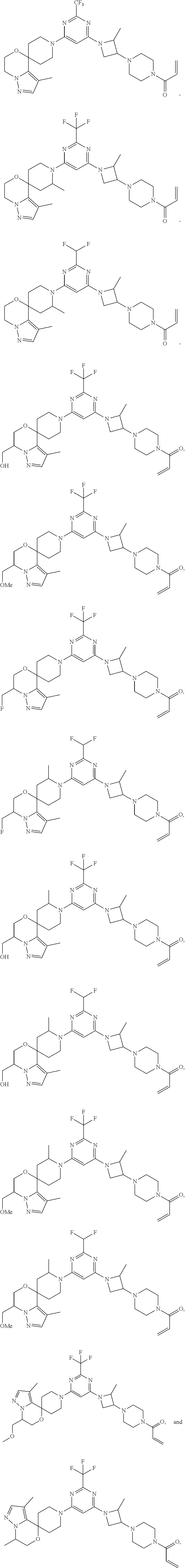

and pharmaceutically acceptable salts thereof.

In some cases, the disclosure provides compounds of Formula (I) or Formula (II) or pharmaceutically acceptable salts thereof, wherein Y′ is thiazolidinyl; p is 2, 3, or 4; and at least one R⁷ is oxo. Examples of such compounds include, but are not limited to:

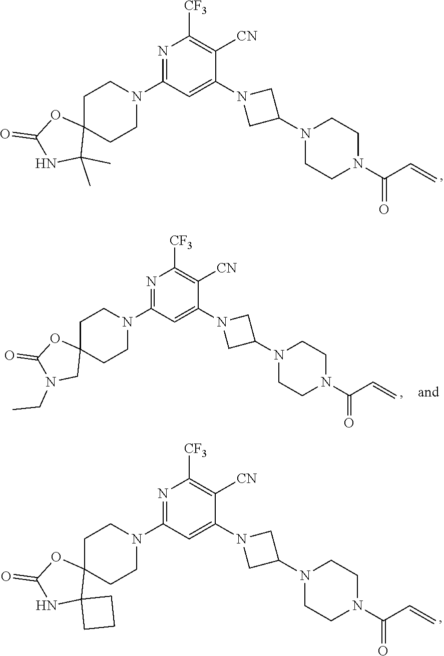 [SMILES: C=CC(=O)N1CCN(C2CN(c3cc(N4CCC5(CC4)OC(=O)NC5(C)C)nc(C(F)(F)F)c3C#N)C2)CC1 ; C=CC(=O)N1CCN(C2CN(c3cc(N4CCC5(CC4)CN(CC)C(=O)O5)nc(C(F)(F)F)c3C#N)C2)CC1 ; C=CC(=O)N1CCN(C2CN(c3cc(N4CCC5(CC4)OC(=O)NC54CCC4)nc(C(F)(F)F)c3C#N)C2)CC1]

and pharmaceutically acceptable salts of the foregoing.

In some cases, the disclosure provides compounds of Formula (I) or Formula (II), or pharmaceutically acceptable salts thereof, wherein Y′ is optionally substituted pyrrolidinyl (e.g., such as with an oxo group to form pyrrolidinone). Examples of such compounds include, but are not limited to:

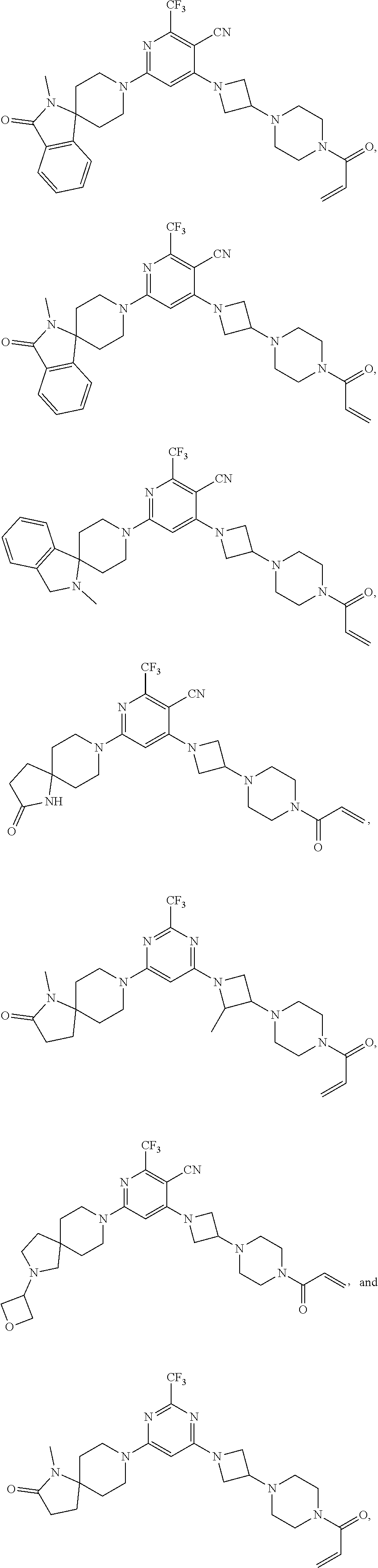

and pharmaceutically acceptable salts of the foregoing.

In some cases, the disclosure provides compounds of Formula (I) or Formula (II), and pharmaceutically acceptable salts thereof, wherein Y′ is optionally substituted dihyropyrrolyl, isoxazolyl, dihydrofuranyl or dihydropyranyl. Examples of such compounds include, but are not limited to:

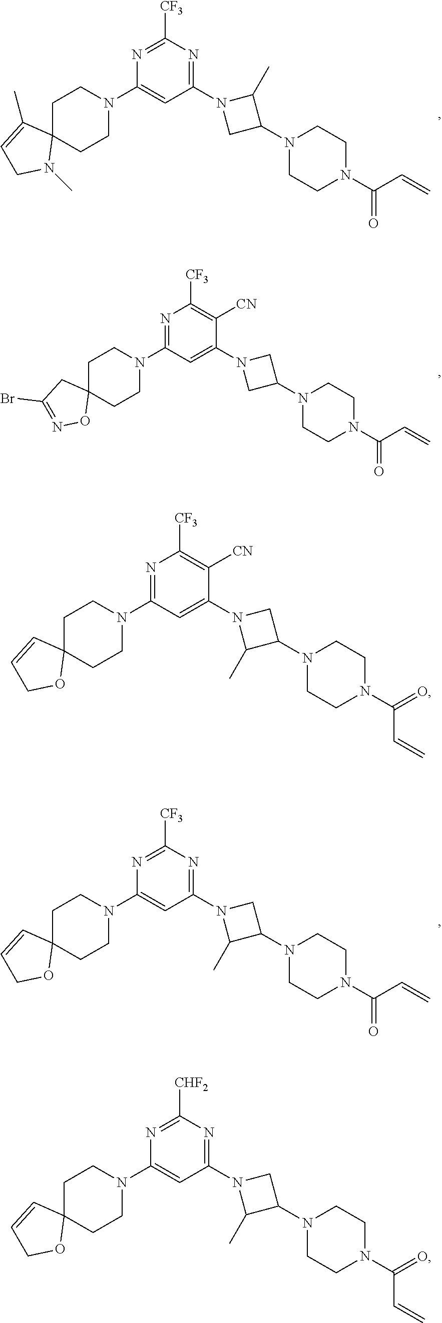 [SMILES: C=CC(=O)N1CCN(C2CN(c3cc(N4CCC5(CC4)C(C)=CCN5C)nc(C(F)(F)F)n3)C2C)CC1 ; C=CC(=O)N1CCN(C2CN(c3cc(N4CCC5(CC4)CC(Br)=NO5)nc(C(F)(F)F)c3C#N)C2)CC1 ; C=CC(=O)N1CCN(C2CN(c3cc(N4CCC5(C=CCO5)CC4)nc(C(F)(F)F)c3C#N)C2C)CC1 ; C=CC(=O)N1CCN(C2CN(c3cc(N4CCC5(C=CCO5)CC4)nc(C(F)(F)F)n3)C2C)CC1 ; C=CC(=O)N1CCN(C2CN(c3cc(N4CCC5(C=CCO5)CC4)nc(C(F)F)n3)C2C)CC1]

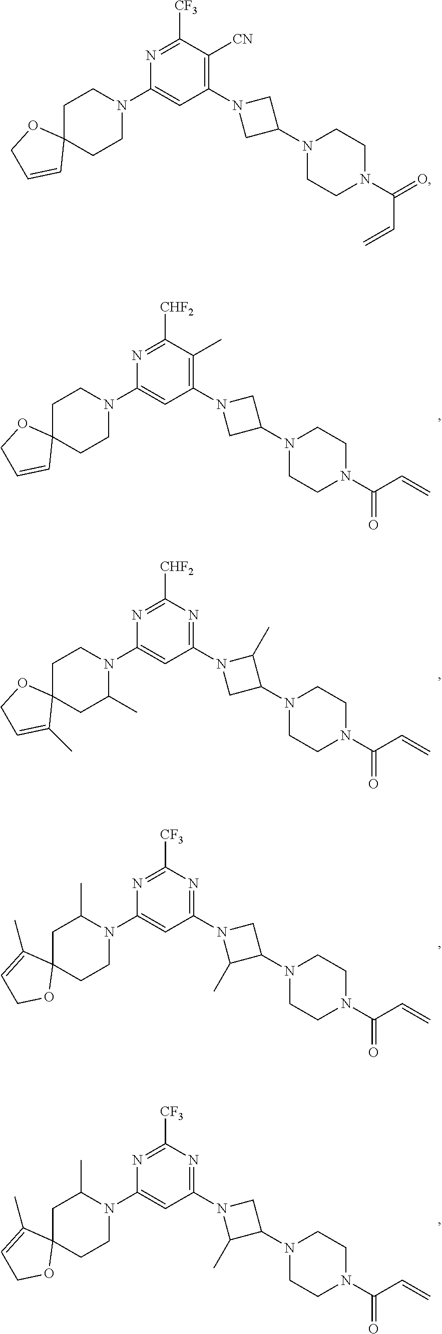 [SMILES: C=CC(=O)N1CCN(C2CN(c3cc(N4CCC5(C=CCO5)CC4)nc(C(F)(F)F)c3C#N)C2)CC1 ; C=CC(=O)N1CCN(C2CN(c3cc(N4CCC5(C=CCO5)CC4)nc(C(F)F)c3C)C2)CC1 ; C=CC(=O)N1CCN(C2CN(c3cc(N4CCC5(CC4C)OCC=C5C)nc(C(F)F)n3)C2C)CC1 ; C=CC(=O)N1CCN(C2CN(c3cc(N4CCC5(CC4C)OCC=C5C)nc(C(F)(F)F)n3)C2C)CC1 ; C=CC(=O)N1CCN(C2CN(c3cc(N4CCC5(CC4C)OCC=C5C)nc(C(F)(F)F)n3)C2C)CC1]

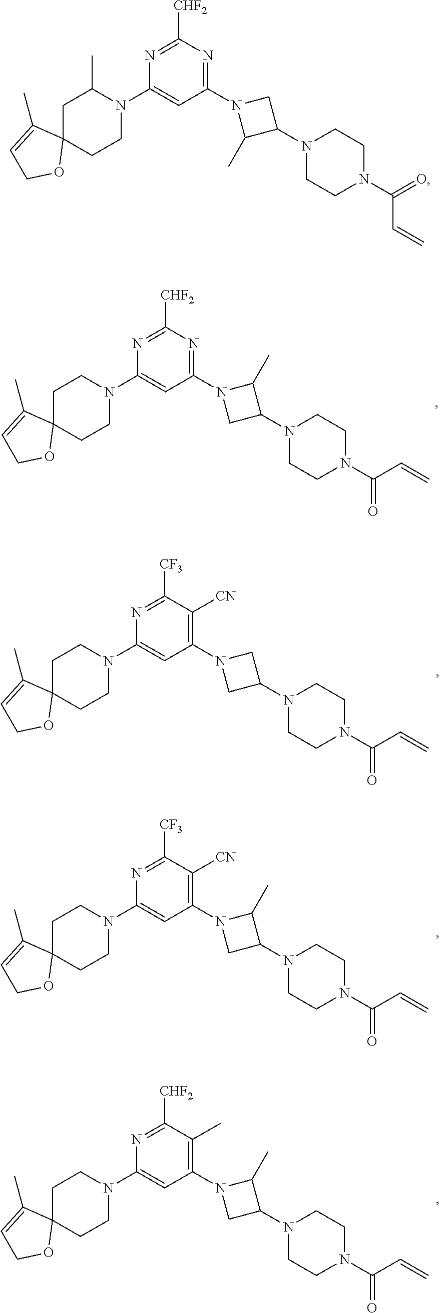 [SMILES: C=CC(=O)N1CCN(C2CN(c3cc(N4CCC5(CC4C)OCC=C5C)nc(C(F)F)n3)C2C)CC1 ; C=CC(=O)N1CCN(C2CN(c3cc(N4CCC5(CC4)OCC=C5C)nc(C(F)F)n3)C2C)CC1 ; C=CC(=O)N1CCN(C2CN(c3cc(N4CCC5(CC4)OCC=C5C)nc(C(F)(F)F)c3C#N)C2)CC1 ; C=CC(=O)N1CCN(C2CN(c3cc(N4CCC5(CC4)OCC=C5C)nc(C(F)(F)F)c3C#N)C2C)CC1 ; C=CC(=O)N1CCN(C2CN(c3cc(N4CCC5(CC4)OCC=C5C)nc(C(F)F)c3C)C2C)CC1]

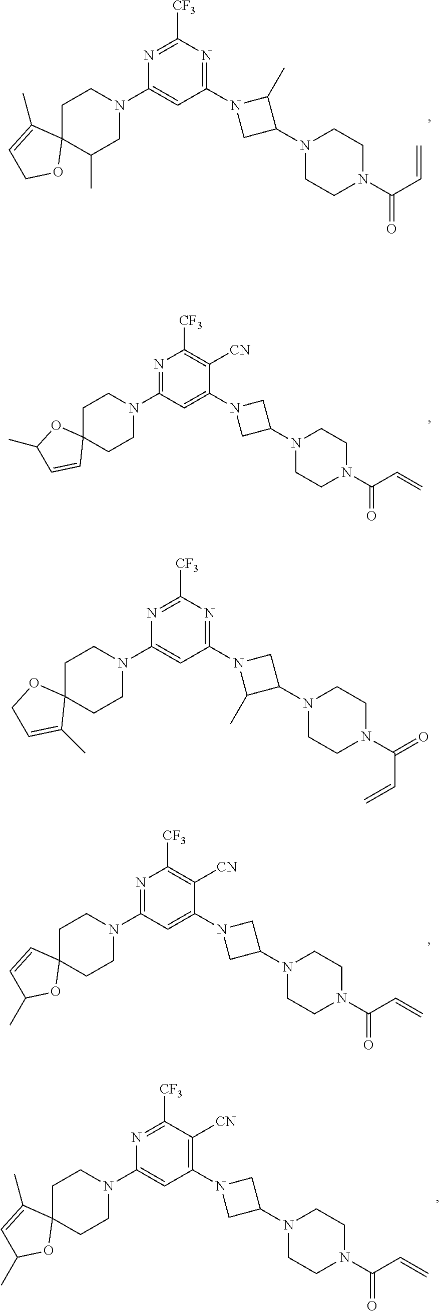

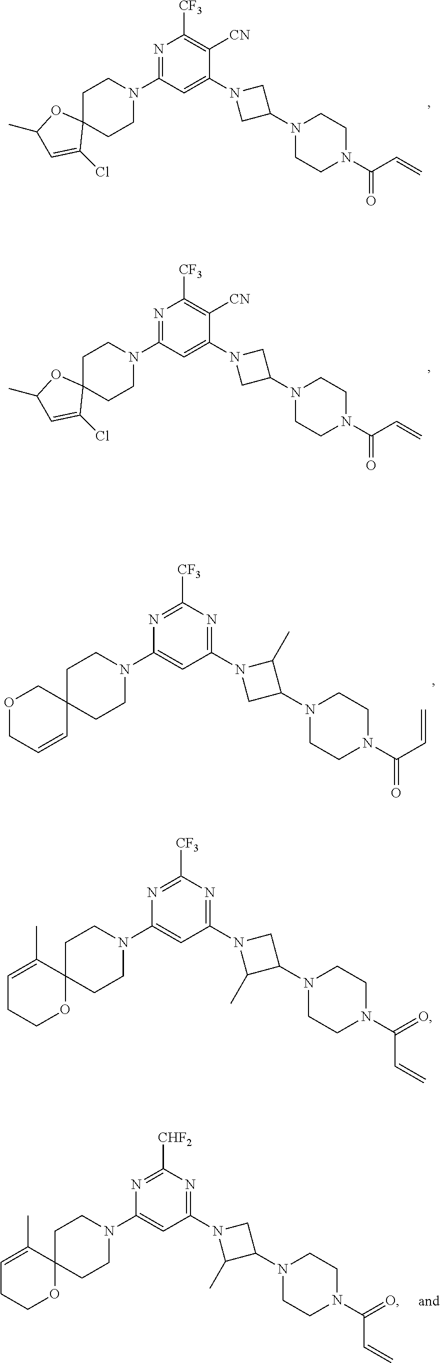

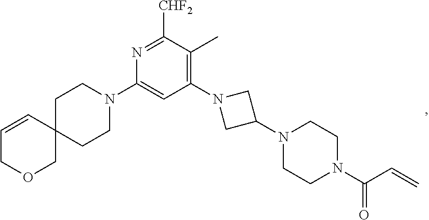 [SMILES: C=CC(=O)N1CCN(C2CN(c3cc(N4CCC5(C=CCOC5)CC4)nc(C(F)F)c3C)C2)CC1]

and pharmaceutically acceptable salts of the foregoing.

In some cases, the compound of Formula (I) or Formula (II) is a compound listed in Table B, or a pharmaceutically acceptable salt thereof.

TABLE B

| Chemical Structure | Name |
|---|---|
| 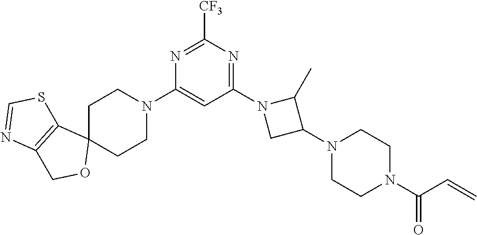 [SMILES: C=CC(=O)N1CCN(C2CN(c3cc(N4CCC5(CC4)OCc4ncsc45)nc(C(F)(F)F)n3)C2C)CC1] | 1-(4-(1-(6-(4H-spiro[furo[3,4-d]thiazole-6,4′- piperidin]-1′-yl)-2-(trifluoromethyl)pyrimidin-4- yl)-2-methylazetidin-3-yl)piperazin-1-yl)prop-2- en-1-one |
| 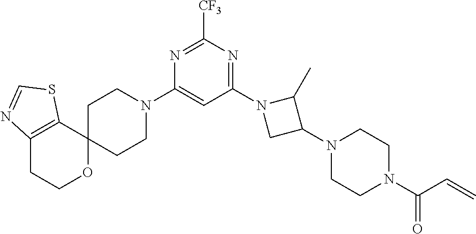 [SMILES: C=CC(=O)N1CCN(C2CN(c3cc(N4CCC5(CC4)OCCc4ncsc45)nc(C(F)(F)F)n3)C2C)CC1] | 1-(4-(1-(6-(6′,7′-dihydrospiro[piperidine-4,4′- pyrano[4,3-d]thiazol]-1-yl)-2- (trifluoromethyl)pyrimidin-4-yl)-2- methylazetidin-3-yl)piperazin-1-yl)prop-2-en-1- one |
| 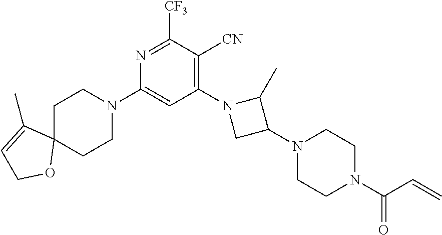 [SMILES: C=CC(=O)N1CCN(C2CN(c3cc(N4CCC5(CC4)OCC=C5C)nc(C(F)(F)F)c3C#N)C2C)CC1] | 4-(3-(4-acryloylpiperazin-1-yl)-2- methylazetidin-1-yl)-6-(4-methyl-1-oxa-8- azaspiro[4.5]dec-3-en-8-yl)-2- (trifluoromethyl)nicotinonitrile |
| 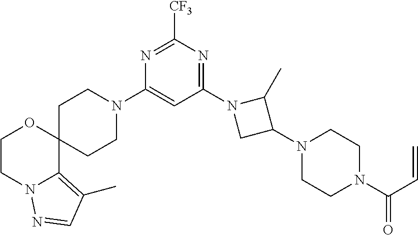 [SMILES: C=CC(=O)N1CCN(C2CN(c3cc(N4CCC5(CC4)OCCn4ncc(C)c45)nc(C(F)(F)F)n3)C2C)CC1] | 1-(4-(2-methyl-1-(6-(3′-methyl-6′,7′- dihydrospiro[piperidine-4 [symbol] 4′-pyrazolo[5,1- c][1,4]oxazin]-1-yl)-2- (trifluoromethyl)pyrimidin-4-yl)azetidin-3- yl)piperazin-1-yl)prop-2-en-1-one |
| 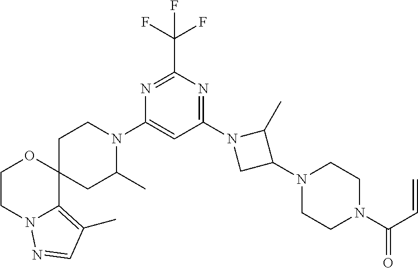 [SMILES: C=CC(=O)N1CCN(C2CN(c3cc(N4CCC5(CC4C)OCCn4ncc(C)c45)nc(C(F)(F)F)n3)C2C)CC1] | 1-(4-(1-(6-(2,3′-dimethyl-6′,7′-dihydro-1H- spiro[piperidine-4,4′-pyrazolo[5,1- c][1,4]oxazin]-1-yl)-2-(trifluoromethyl)-4- pyrimidinyl)-2-methyl-3-azetidinyl)-1- piperazinyl)-2-propen-1-one |
| 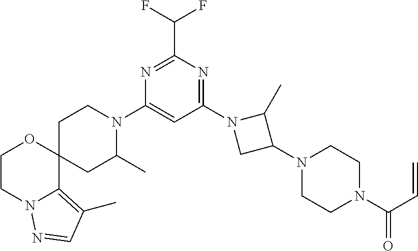 [SMILES: C=CC(=O)N1CCN(C2CN(c3cc(N4CCC5(CC4C)OCCn4ncc(C)c45)nc(C(F)F)n3)C2C)CC1] | 1-(4-(1-(2-(difluoromethyl)-6-(2,3′-dimethyl- 6′,7′-dihydro-1H-spiro[piperidine-4,4′- pyrazolo[5,1-c][1,4]oxazin]-1-yl)-4- pyrimidinyl)-2-methyl-3-azetidinyl)-1- piperazinyl)-2-propen-1-one |
| 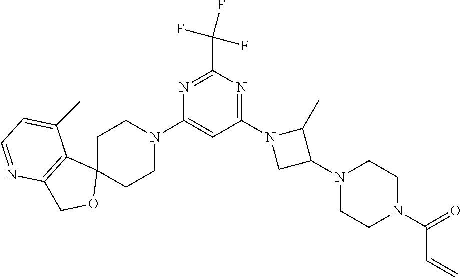 [SMILES: C=CC(=O)N1CCN(C2CN(c3cc(N4CCC5(CC4)OCc4nccc(C)c45)nc(C(F)(F)F)n3)C2C)CC1] | 1-(4-(2-methyl-1-(6-(4-methyl-1′H,7H- spiro[furo[3,4-b]pyridine-5,4′-piperidin]-1′-yl)- 2-(trifluoromethyl)-4-pyrimidinyl)-3-azetidinyl)- 1-piperazinyl)-2-propen-1-one |
| 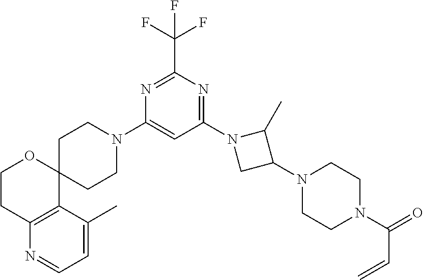 [SMILES: C=CC(=O)N1CCN(C2CN(c3cc(N4CCC5(CC4)OCCc4nccc(C)c45)nc(C(F)(F)F)n3)C2C)CC1] | 1-(4-methyl-1-(6-(4′-methyl-7′,8′- dihydrospiro[piperidine-4,5′-pyrano[4,3- b]pyridin]-1-yl)-2-(trifluoromethyl)pyrimidin- 4-yl)azetidin-3-yl)piperazin-1-yl)prop-2-en-1- one |
| 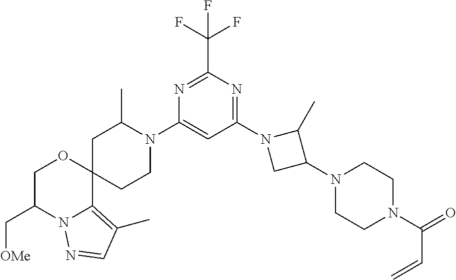 [SMILES: C=CC(=O)N1CCN(C2CN(c3cc(N4CCC5(CC4C)OCC(COC)n4ncc(C)c45)nc(C(F)(F)F)n3)C2C)CC1] | 1-(4-(1-(6-((7′-(methoxymethyl)-2,3′-dimethyl- 6′,7′-dihydrospiro[piperidine-4,4′-pyrazolo[5,1- c][1,4]oxazin]-1-yl)-2- (trifluoromethyl)pyrimidin-4-yl)-2- methylazetidin-3-yl)pipearzin-1-yl)prop-2-en-1- one |
| 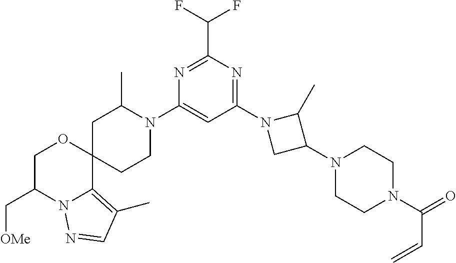 [SMILES: C=CC(=O)N1CCN(C2CN(c3cc(N4CCC5(CC4C)OCC(COC)n4ncc(C)c45)nc(C(F)F)n3)C2C)CC1] | 1-(4-(1-(2-(difluoromethyl)-6-(7′- (methoxymethyl)-2,3′-dimethyl-6′,7′- dihydrospiro[piperidine-4,4′-pyrazolo[5,1- c][1,4]oxazin]-1-yl)pyrimidin-4-yl)-2- methylazetidin-3-yl)piperazin-1-yl)prop-2-en- 1-one |
| 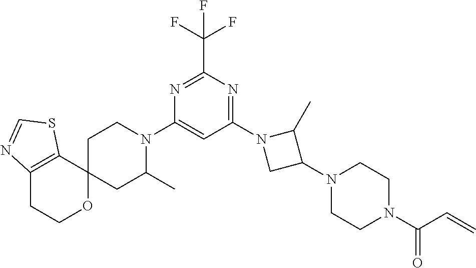 [SMILES: C=CC(=O)N1CCN(C2CN(c3cc(N4CCC5(CC4C)OCCc4ncsc45)nc(C(F)(F)F)n3)C2C)CC1] | 1-(4-(2-methyl-1-(6-(2-methyl-6′,7′- dihydrospiro[piperidine-4,4′-pyrano[4,3- d]thiazol]-1-yl)-2-(trifluoromethyl)pyrimidin-4- yl)azetidin-3-yl)piperazin-1-yl)prop-2-en-1-one |

[symbol]

indicates data missing or illegible when filed

In some cases, the disclosure provides a compound listed in Table C, below. If the stereochemistry of a structure or a portion of a structure in Table C is not explicitly shown (e.g., such as with dashed or bold lines), then the structure or portion of structure is either achiral or interpreted as being any of the possible stereoisomers of the structure or portion of the structure. In cases in which the stereochemistry of the structure or portion of the structure in Table C is explicitly shown, a single stereoisomer of the structure or portion of a structure is represented, with the understanding that the stereochemistry of the structure or portion of the structure shown in Table C has been arbitrarily assigned.

TABLE C

| Compound No. | Chemical Structure | Name |
|---|---|---|
| 3-001 | 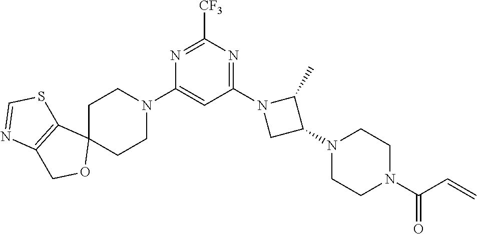 [SMILES: C=CC(=O)N1CCN([C@@H]2CN(c3cc(N4CCC5(CC4)OCc4ncsc45)nc(C(F)(F)F)n3)[C@@H]2C)CC1] | 1-(4-((2R,3R)-1-(6-(4H-spiro[furo[3,4- d]thiazole-6,4′-piperidin]-1′-yl)-2- (trifluoromethyl)pyrimidin-4-yl)-2- methylazetidin-3-yl)piperazin-1-yl) prop-2-en-1-one |
| 3-002 | 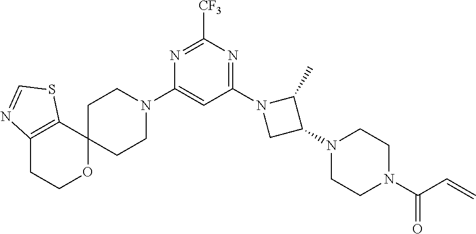 [SMILES: C=CC(=O)N1CCN([C@@H]2CN(c3cc(N4CCC5(CC4)OCCc4ncsc45)nc(C(F)(F)F)n3)[C@@H]2C)CC1] | 1-(4-((2R,3R)-1-(6-(6′,7′- dihydrospiro[piperidine-4,4′-pyrano[4,3- d]thiazol]-1-yl)-2-(trifluoromethyl)pyrimidin- 4-yl)-2-methylazetidin-3-yl)piperazin-1- yl)prop-2-en-1-one |
| 3-003 | 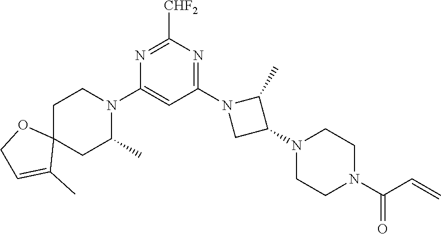 [SMILES: C=CC(=O)N1CCN([C@@H]2CN(c3cc(N4CCC5(C[C@H]4C)OCC=C5C)nc(C(F)F)n3)[C@@H]2C)CC1] | 1-(4-((2R,3R)-1-(2-(difluoromethyl)-6-((7R)- 4,7-dimethyl-1-oxa-8-azaspiro[4.5]dec-3-en- 8-yl)pyrimidin-4-yl)-2-methylazetidin-3- yl)piperazin-1-yl)prop-2-en-1-one |
| 3-004 | 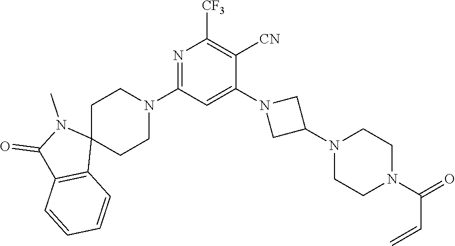 [SMILES: C=CC(=O)N1CCN(C2CN(c3cc(N4CCC5(CC4)c4ccccc4C(=O)N5C)nc(C(F)(F)F)c3C#N)C2)CC1] | 4-(3-(4-acryloylpiperazin-1-yl)azetidin-1-yl)- 6-(2-methyl-3-oxospiro[isoindoline-1,4′- piperidin]-1′-yl)-2- (trifluoromethyl)nicotinonitrile |
| 3-005 | 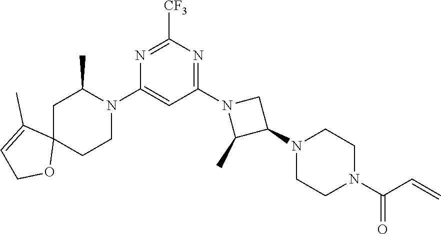 [SMILES: C=CC(=O)N1CCN([C@@H]2CN(c3cc(N4CCC5(C[C@H]4C)OCC=C5C)nc(C(F)(F)F)n3)[C@@H]2C)CC1] | 1-(4-((2R,3R)-1-(6-((7R)-4,7-dimethyl-1- oxa-8-azaspiro[4.5]dec-3-en-8-yl)-2- (trifluoromethyl)pyrimidin-4-yl)-2- methylazetidin3-yl)piperazin-1-yl)prop-2- en-1-one |
| 3-006 | 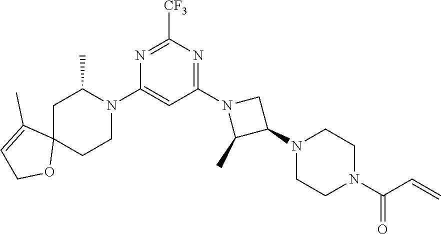 [SMILES: C=CC(=O)N1CCN([C@@H]2CN(c3cc(N4CCC5(C[C@@H]4C)OCC=C5C)nc(C(F)(F)F)n3)[C@@H]2C)CC1] | 1-(4-((2R,3R)-1-(6-((7S)-4,7-dimethyl-1- oxa-8-azaspiro[4.5]dec-3-en-8-yl)-2- (trifluoromethyl)pyrimidin-4-yl)-2- methylazetidin-3-yl)piperazin-1-yl)prop- 2-en-1-one |
| 3-007 | 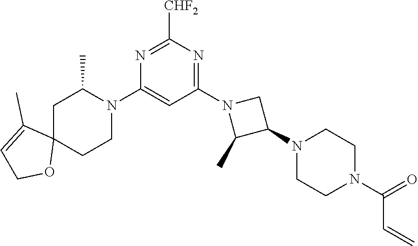 [SMILES: C=CC(=O)N1CCN([C@@H]2CN(c3cc(N4CCC5(C[C@@H]4C)OCC=C5C)nc(C(F)F)n3)[C@@H]2C)CC1] | 1-(4-((2R,3R)-1-(2-(difluoromethyl)-6-((7S)- 4,7-dimethyl-1-oxa-8-azaspiro[4.5]dec-3-en- 8-yl)pyrimidin-4-yl)-2-methylazetidin-3- yl)piperazin-1-yl)prop-2-en-1-one |
| 3-008 | 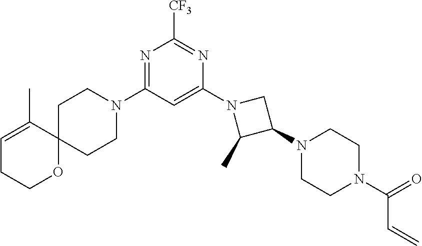 [SMILES: C=CC(=O)N1CCN([C@@H]2CN(c3cc(N4CCC5(CC4)OCCC=C5C)nc(C(F)(F)F)n3)[C@@H]2C)CC1] | 1-(4-((2R,3R)-2-methyl-1-(6-(5-methyl-1- oxa-9-azaspiro[5.5]undec-4-en-9-yl)-2- (trifluoromethyl)pyrimidin-4-yl)azetidin-3- yl)piperazin-1-yl)prop-2-en-1-one |
| 3-009 | 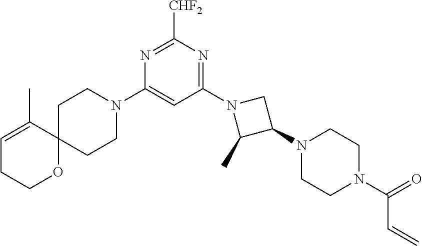 [SMILES: C=CC(=O)N1CCN([C@@H]2CN(c3cc(N4CCC5(CC4)OCCC=C5C)nc(C(F)F)n3)[C@@H]2C)CC1] | 1-(4-((2R,3R)-1-(2-(difluoromethyl)-6-(5- methyl-1-oxa-9-azaspiro[5.5]undec-4-en-9- yl)pyrimidin-4-yl)-2-methylazetidin-3- yl)piperazin-1-yl)prop-2-en-1-one |
| 3-010 | 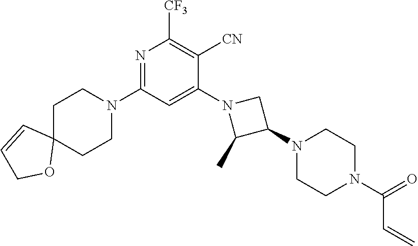 [SMILES: C=CC(=O)N1CCN([C@@H]2CN(c3cc(N4CCC5(C=CCO5)CC4)nc(C(F)(F)F)c3C#N)[C@@H]2C)CC1] | 4-((2R,3R)-3-(4-acryloylpiperazin-1-yl)-2- methylazetidin-1-yl)-6-(1-oxa-8- azaspiro[4.5]dec-3-en-8-yl)-2- (trifluoromethyl)nicotinonitrile |
| 3-011 | 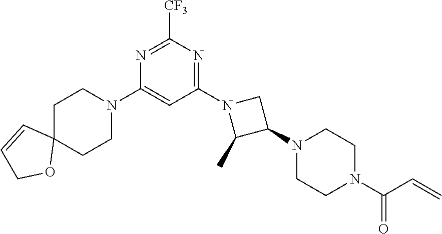 [SMILES: C=CC(=O)N1CCN([C@@H]2CN(c3cc(N4CCC5(C=CCO5)CC4)nc(C(F)(F)F)n3)[C@@H]2C)CC1] | 1-(4-((2R,3R)-1-(6-(1-oxa-8- azaspiro[4.5]dec-3-en-8-yl)-2- (trifluoromethyl)pyrimidin-4-yl)-2- methylazetidin-3-yl)piperazin-1-yl)prop-2- en-1-one |
| 3-012 | 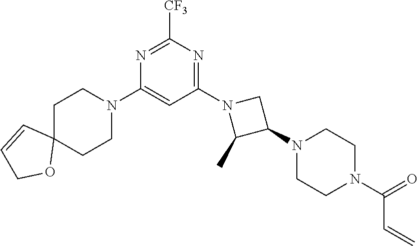 [SMILES: C=CC(=O)N1CCN([C@@H]2CN(c3cc(N4CCC5(C=CCO5)CC4)nc(C(F)(F)F)n3)[C@@H]2C)CC1] | 1-(4-((2R,3R)-1-(2-(difluoromethyl)-6-(1- oxa-8-azaspiro[4.5]dec-3-en-8-yl)pyrimidin- 4-yl)-2-methylazetidin-3-yl)piperazin-1- yl)prop-2-en-1-one |
| 3-013 | 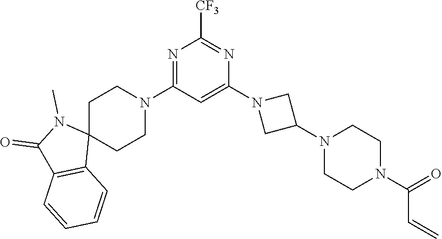 [SMILES: C=CC(=O)N1CCN(C2CN(c3cc(N4CCC5(CC4)c4ccccc4C(=O)N5C)nc(C(F)(F)F)n3)C2)CC1] | 1′-(6-(3-(4-acryloylpiperazin-1-yl)azetidin-1- yl)-2-(trifluoromethyl)pyrimidin-4-yl)-2- methylspiro[isoindoline-1,4′-piperidin]-3-one |
| 3-014 | 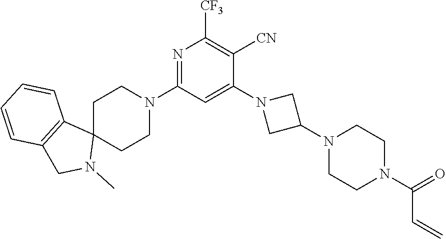 [SMILES: C=CC(=O)N1CCN(C2CN(c3cc(N4CCC5(CC4)c4ccccc4CN5C)nc(C(F)(F)F)c3C#N)C2)CC1] | 4-(3-(4-acryloylpiperazin-1-yl)azetidin-1-yl)- 6-(2-methylspiro[isoindoline-1,4′-piperidin]- 1′-yl)-2-(trifluoromethyl)nicotinonitrile |
| 3-015 | 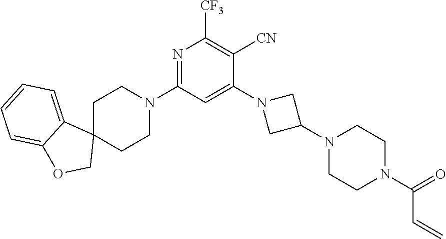 [SMILES: C=CC(=O)N1CCN(C2CN(c3cc(N4CCC5(CC4)COc4ccccc45)nc(C(F)(F)F)c3C#N)C2)CC1] | 4-(3-(4-acryloylpiperazin-1-yl)azetidin-1-yl)- 6-(2H-spiro[benzofuran-3,4′-piperidin]-1′-yl)- 2-(trifluoromethyl)nicotinonitrile |
| 3-016 | 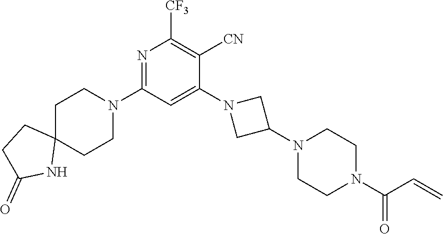 [SMILES: C=CC(=O)N1CCN(C2CN(c3cc(N4CCC5(CCC(=O)N5)CC4)nc(C(F)(F)F)c3C#N)C2)CC1] | 4-(3-(4-acryloylpiperazin-1-yl)azetidin-1-yl)- 6-(2-oxo-1,8-diazaspiro[4.5]decan-8-yl)-2- (trifluoromethyl)nicotinonitrile |
| 3-017 | 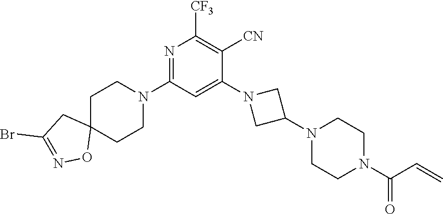 [SMILES: C=CC(=O)N1CCN(C2CN(c3cc(N4CCC5(CC4)CC(Br)=NO5)nc(C(F)(F)F)c3C#N)C2)CC1] | 4-(3-(4-acryloylpiperazin-1-yl)azetidin-1-yl)- 6-(3-bromo-1-oxa-2,8-diazaspiro[4.5]dec-2- en-8-yl)-2-(trifluoromethyl)nicotinonitrile |
| 3-018 | 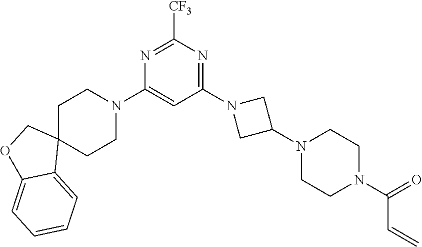 [SMILES: C=CC(=O)N1CCN(C2CN(c3cc(N4CCC5(CC4)COc4ccccc45)nc(C(F)(F)F)n3)C2)CC1] | 1-(4-(1-(6-(2H-spiro[benzofuran-3,4′- piperidin]-1′-yl)-2- (trifluoromethyl)pyrimidin-4-yl)azetidin-3- yl)piperazin-1-yl)prop-2-en-1-one |
| 3-019 | 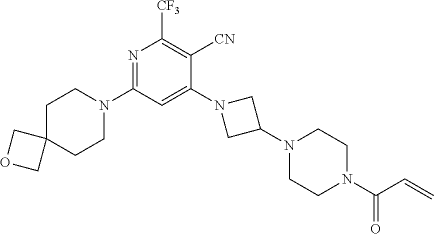 [SMILES: C=CC(=O)N1CCN(C2CN(c3cc(N4CCC5(CC4)COC5)nc(C(F)(F)F)c3C#N)C2)CC1] | 4-(3-(4-acryloylpiperazin-1-yl)azetidin-1-yl)- 6-(2-oxa-7-azaspiro[3.5]nonan-7-yl)-2- (trifluoromethyl)nicotinonitrile |
| 3-020 | 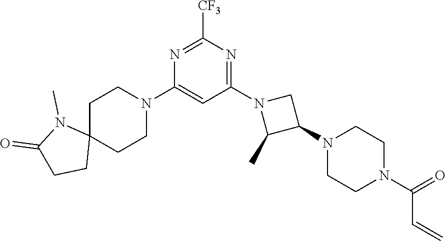 [SMILES: C=CC(=O)N1CCN([C@@H]2CN(c3cc(N4CCC5(CCC(=O)N5C)CC4)nc(C(F)(F)F)n3)[C@@H]2C)CC1] | 8-(6-((2R,3R)-3-(4-acryloylpiperazin-1-yl)-2- methylazetidin-1-yl)-2- (trifluoromethyl)pyrimidin-4-yl)-1-methyl- 1,8-diazaspiro[4.5]decan-2-one |
| 3-021 | 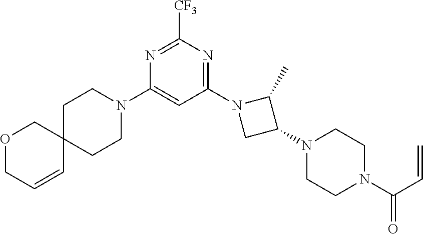 [SMILES: C=CC(=O)N1CCN([C@@H]2CN(c3cc(N4CCC5(C=CCOC5)CC4)nc(C(F)(F)F)n3)[C@@H]2C)CC1] | 1-(4-((2R,3R)-1-(6-(2-oxa-9- azaspiro[5.5]undec-4-en-9-yl)-2- (trifluoromethyl)pyrimidin-4-yl)-2- methylazetidin-3-yl)piperazin-1-yl)prop-2- en-1-one |
| 3-022 | 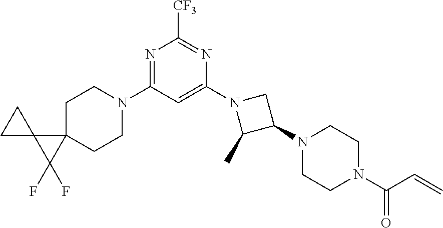 [SMILES: C=CC(=O)N1CCN([C@@H]2CN(c3cc(N4CCC5(CC4)C(F)(F)C54CC4)nc(C(F)(F)F)n3)[C@@H]2C)CC1] | 1-((2R,3R)-1-(6-(10,10-difluoro-7- azadispiro[2.0.5⁴.1³]decan-7-yl)-2- (trifluoromethyl)pyrimidin-4-yl)-2- methylazetidin-3-yl)piperazin-1-yl)prop-2- en-1-one |
| 3-023 | 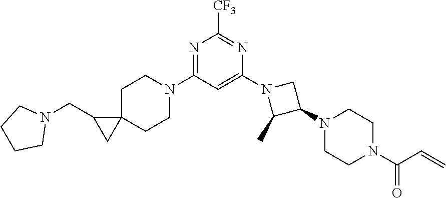 [SMILES: C=CC(=O)N1CCN([C@@H]2CN(c3cc(N4CCC5(CC4)CC5CN4CCCC4)nc(C(F)(F)F)n3)[C@@H]2C)CC1] | 1-(4-((2R,3R)-2-methyl-1-(6-(1-(pyrrolidin- 1-ylmethyl)-6-azaspiro[2.5]octan-6-yl)-2- (trifluoromethyl)pyrimidin-4-yl)azetidin-3- yl)piperazin-1-yl)prop-2-en-1-one |
| 3-024 | 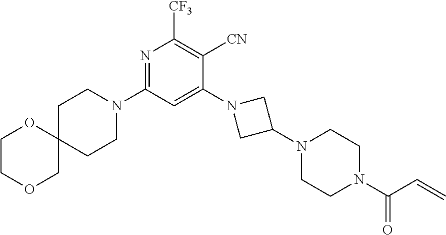 [SMILES: C=CC(=O)N1CCN(C2CN(c3cc(N4CCC5(CC4)COCCO5)nc(C(F)(F)F)c3C#N)C2)CC1] | 4-(3-(4-acryloylpiperazin-1-yl)azetidin-1-yl)- 6-(1,4-dioxa-9-azaspiro[5.5]undecan-9-yl)-2- (trifluoromethyl)nicotinonitrile |
| 3-025 | 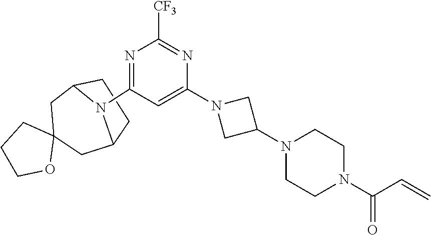 [SMILES: C=CC(=O)N1CCN(C2CN(c3cc(N4C5CCC4CC4(CCCO4)C5)nc(C(F)(F)F)n3)C2)CC1] | 1-(4-(1-(6-(dihydro-3′H-8- azaspiro[3.2.1]octane-3,2′-furan]-8- yl)-2-(trifluoromethyl)pyrimidin-4- yl)azetidin-3-yl)piperazin-1-yl)prop-2-en-1- one |
| 3-026 | 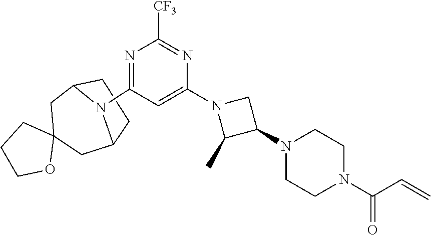 [SMILES: C=CC(=O)N1CCN([C@@H]2CN(c3cc(N4C5CCC4CC4(CCCO4)C5)nc(C(F)(F)F)n3)[C@@H]2C)CC1] | 1-(4-((2R,3R)-1-(6-(dihydro-3′H-8- azaspiro[bicyclo[3.2.1]octane-3,2′-furan]-8- yl)-2-(trifluoromethyl)pyrimidin-4-yl)-2- methylazetidin-3-yl)piperazin-1-yl)prop-2- en-1-one |
| 3-027 | 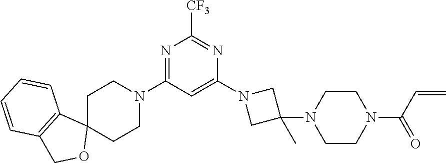 [SMILES: C=CC(=O)N1CCN(C2(C)CN(c3cc(N4CCC5(CC4)OCc4ccccc45)nc(C(F)(F)F)n3)C2)CC1] | 1-(4-(1-(6-(3H-spiro[isobenzofuran-1,4′- piperidin]-1′-yl)-2- (trifluoromethyl)pyrimidin-4-yl)-3- methylazetidin-3-yl)piperazin-1-yl)prop-2- en-1-one |
| 3-028 | 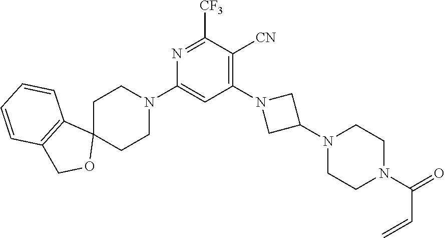 [SMILES: C=CC(=O)N1CCN(C2CN(c3cc(N4CCC5(CC4)OCc4ccccc45)nc(C(F)(F)F)c3C#N)C2)CC1] | 4-(3-(4-acryloylpiperazin-1-yl)azetidin-1-yl)- 6-(3H-spiro[isobenzofuran-1,4′-piperidin]-1′- yl)-2-(trifluoromethyl)nicotinonitrile |
| 3-029 | 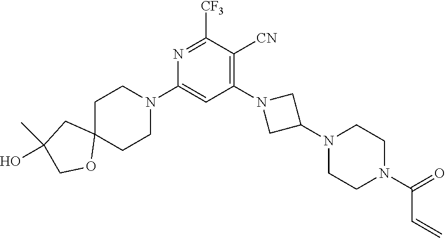 [SMILES: C=CC(=O)N1CCN(C2CN(c3cc(N4CCC5(CC4)CC(C)(O)CO5)nc(C(F)(F)F)c3C#N)C2)CC1] | 4-(3-(4-acryloylpiperazin-1-yl)azetidin-1-yl)- 6-(3-hydroxy-3-methyl-1-oxa-8- azaspiro[4.5]decan-8-yl)-2- (trifluoromethyl)nicotinonitrile |
| 3-030 | 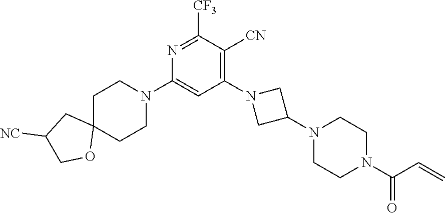 [SMILES: C=CC(=O)N1CCN(C2CN(c3cc(N4CCC5(CC4)CC(C#N)CO5)nc(C(F)(F)F)c3C#N)C2)CC1] | 8-(4-(3-(4-acryloylpiperazin-1-yl)azetidin-1- yl)-5-cyano-6-(trifluoromethyl)pyridin-2-yl)- 1-oxa-8-azaspiro[4.5]decane-3-carbonitrile |
| 3-031 | 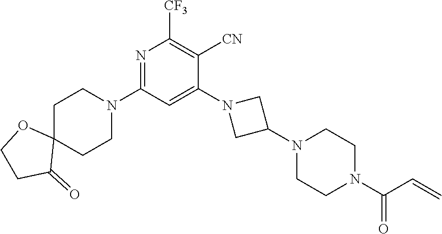 [SMILES: C=CC(=O)N1CCN(C2CN(c3cc(N4CCC5(CC4)OCCC5=O)nc(C(F)(F)F)c3C#N)C2)CC1] | 4-(3-(4-acryloylpiperazin-1-yl)azetidin-1-yl)- 6-(4-oxo-1-oxa-8-azaspiro[4.5]decan-8-yl)-2- (trifluoromethyl)nicotinonitrile |
| 3-032 | 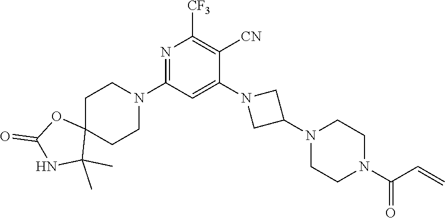 [SMILES: C=CC(=O)N1CCN(C2CN(c3cc(N4CCC5(CC4)OC(=O)NC5(C)C)nc(C(F)(F)F)c3C#N)C2)CC1] | 4-(3-(4-acryloylpiperazin-1-yl)azetidin-1-yl)- 6-(4 [symbol] 4-dimethyl-2-oxo-1-oxa-3,8- diazaspiro[4.5]decan-8-yl)-2- (trifluoromethyl)nicotinonitrile |
| 3-033 | 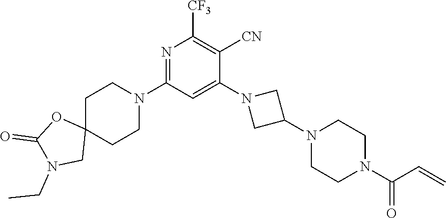 [SMILES: C=CC(=O)N1CCN(C2CN(c3cc(N4CCC5(CC4)CN(CC)C(=O)O5)nc(C(F)(F)F)c3C#N)C2)CC1] | 4-(3-(4-acryloylpiperazin-1-yl)azetidin-1-yl)- 6-(3-ethyl-2-oxo-1-oxa-3,8- diazaspiro[4.5]decan-8-yl)-2- (trifluoromethyl)nicotinonitrile |
| 3-034 | 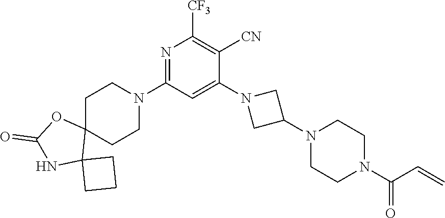 [SMILES: C=CC(=O)N1CCN(C2CN(c3cc(N4CCC5(CC4)OC(=O)NC54CCC4)nc(C(F)(F)F)c3C#N)C2)CC1] | 4-(3-(4-acryloylpiperazin-1-yl)azetidin-1-yl)- 6-(12-oxo-11-oxa-8,13- diazadispiro[3.0.55.34]tridecan-8-yl)-2- (trifluoromethyl)nicotinonitrile |
| 3-035 | 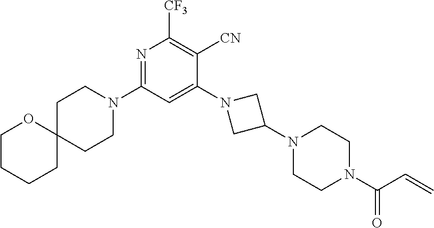 [SMILES: C=CC(=O)N1CCN(C2CN(c3cc(N4CCC5(CCCCO5)CC4)nc(C(F)(F)F)c3C#N)C2)CC1] | 4-(3-(4-acryloylpiperazin-1-yl)azetidin-1-yl)- 6-(1-oxa-9-azaspiro[5.5]undecan-9-yl)-2- (trifluoromethyl)nicotinonitrile |
| 3-036 | 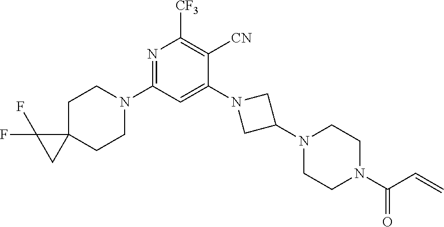 [SMILES: C=CC(=O)N1CCN(C2CN(c3cc(N4CCC5(CC4)CC5(F)F)nc(C(F)(F)F)c3C#N)C2)CC1] | 4-(3-(4-acryloylpiperazin-1-yl)azetidin-1-yl)- 6-(1,1-difluoro-6-azaspiro[2.5]octan-6-yl)-2- (trifluoromethyl)nicotinonitrile |
| 3-037 | 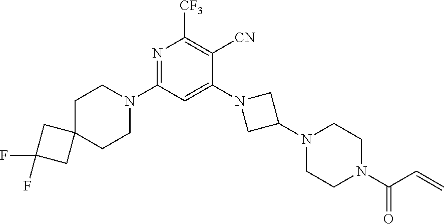 [SMILES: C=CC(=O)N1CCN(C2CN(c3cc(N4CCC5(CC4)CC(F)(F)C5)nc(C(F)(F)F)c3C#N)C2)CC1] | 4-(3-(4-acryloylpiperazin-1-yl)azetidin-1-yl)- 6-(2,2-difluoro-7-azaspiro[3.5]nonan-7-yl)-2- (trifluoromethyl)nicotinonitrile |
| 3-038 | 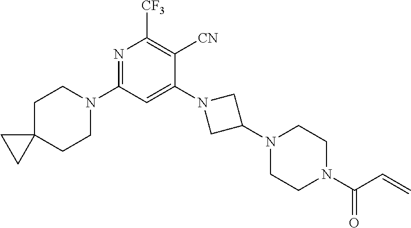 [SMILES: C=CC(=O)N1CCN(C2CN(c3cc(N4CCC5(CC4)CC5)nc(C(F)(F)F)c3C#N)C2)CC1] | 4-(3-(4-acryloylpiperazin-1-yl)azetidin-1-yl)- 6-(6-azaspiro[2.5]octan-6-yl)-2- (trifluoromethyl)nicotinonitrile |
| 3-039 | 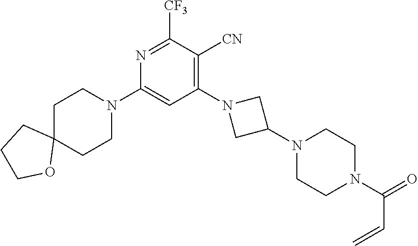 [SMILES: C=CC(=O)N1CCN(C2CN(c3cc(N4CCC5(CCCO5)CC4)nc(C(F)(F)F)c3C#N)C2)CC1] | 4-(3-(4-acryloylpiperazin-1-yl)azetidin-1-yl)- 6-(1-oxa-8-azaspiro[4.5]decan-8-yl)-2- (trifluoromethyl)nicotinonitrile |
| 3-040 | 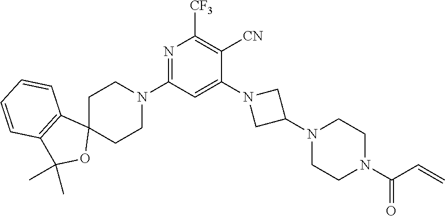 [SMILES: C=CC(=O)N1CCN(C2CN(c3cc(N4CCC5(CC4)OC(C)(C)c4ccccc45)nc(C(F)(F)F)c3C#N)C2)CC1] | 4-(3-(4-acryloylpiperazin-1-yl)azetidin-1-yl)- 6-(3,3-dimethyl-3H-spiro[isobenzofuran-1,4′- piperidin]-1′-yl)-2- (trifluoromethyl)nicotinonitrile |
| 3-041 | 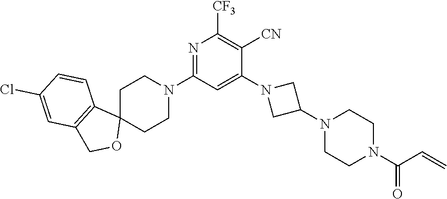 [SMILES: C=CC(=O)N1CCN(C2CN(c3cc(N4CCC5(CC4)OCc4cc(Cl)ccc45)nc(C(F)(F)F)c3C#N)C2)CC1] | 4-(3-(4-acryloylpiperazin-1-yl)azetidin-1-yl)- 6-(5-chloro-3H-spiro[isobenzofuran-1,4′- piperidin]-1′-yl)-2- (trifluoromethyl)nicotinonitrile |
| 3-042 | 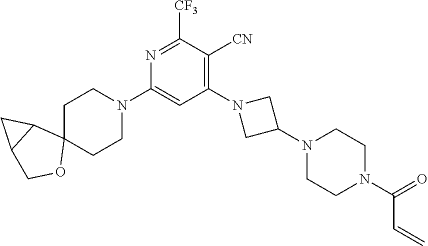 [SMILES: C=CC(=O)N1CCN(C2CN(c3cc(N4CCC5(CC4)OCC4CC45)nc(C(F)(F)F)c3C#N)C2)CC1] | 4-(3-(4-acryloylpiperazin-1-yl)azetidin-1-yl)- 6-(3-oxaspiro[bicyclo[3.1.0]hexane-2,4′- piperidin]-1′-yl)-2- (trifluoromethyl)nicotinonitrile |
| 3-043 | 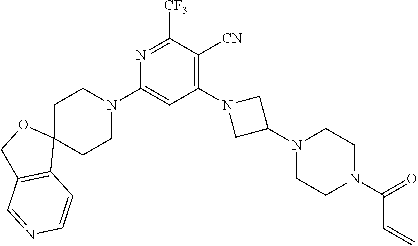 [SMILES: C=CC(=O)N1CCN(C2CN(c3cc(N4CCC5(CC4)OCc4cnccc45)nc(C(F)(F)F)c3C#N)C2)CC1] | 4-(3-(4-acryloylpiperazin-1-yl)azetidin-1-yl)- 6-(3H-spiro[furo[3,4-c]pyridine-1,4′- piperidin]-1′-yl)-2- (trifluoromethyl)nicotinonitrile |
| 3-044 | 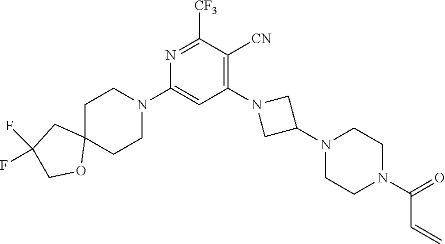 [SMILES: C=CC(=O)N1CCN(C2CN(c3cc(N4CCC5(CC4)CC(F)(F)CO5)nc(C(F)(F)F)c3C#N)C2)CC1] | 4-(3-(4-acryloylpiperazin-1-yl)azetidin-1-yl)- 6-(3,3-difluoro-1-oxa-8-azaspiro[4.5]decan- 8-yl)-2-(trifluoromethyl)nicotinonitrile |
| 3-045 | 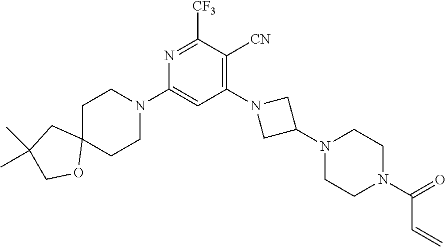 [SMILES: C=CC(=O)N1CCN(C2CN(c3cc(N4CCC5(CC4)CC(C)(C)CO5)nc(C(F)(F)F)c3C#N)C2)CC1] | 4-(3-(4-acryloylpiperazin-1-yl)azetidin-1-yl)- 6-(3,3-dimethyl-1-oxa-8-azaspiro[4.5]decan- 8-yl)-2-(trifluoromethyl)nicotinonitrile |
| 3-046 | 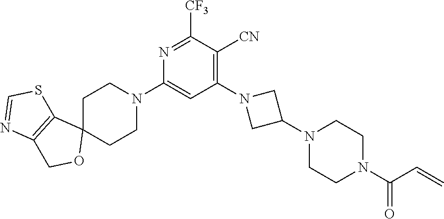 [SMILES: C=CC(=O)N1CCN(C2CN(c3cc(N4CCC5(CC4)OCc4ncsc45)nc(C(F)(F)F)c3C#N)C2)CC1] | 4-(3-(4-acryloylpiperazin-1-yl)azetidin-1-yl)- 6-(4H-spiro[furo[3,4-d]thiazole-6,4′- piperidin]-1′-yl)-2- (triflluoromethyl)nicotinonitrile |
| 3-047 | 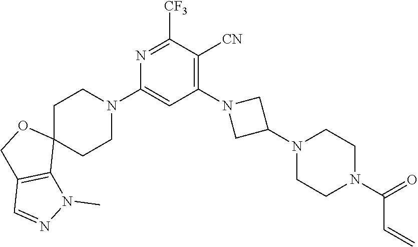 [SMILES: C=CC(=O)N1CCN(C2CN(c3cc(N4CCC5(CC4)OCc4cnn(C)c45)nc(C(F)(F)F)c3C#N)C2)CC1] | 1-(4-(1-(6-(1-methyl-1,4- dihydrospiro[furo[3,4-c]pyrazole-6,4′- piperidin]-1′-yl)-2- (trifluoromethyl)pyrimidin-4-yl)azetidin-3- yl)piperazin-1-yl)prop-2-en-1-one |
| 3-048 | 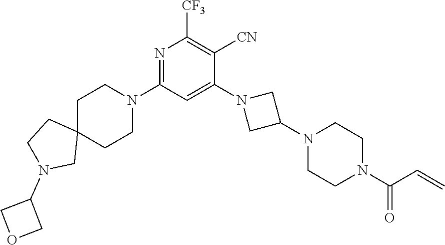 [SMILES: C=CC(=O)N1CCN(C2CN(c3cc(N4CCC5(CC4)CCN(C4COC4)C5)nc(C(F)(F)F)c3C#N)C2)CC1] | 4-(3-(4-acryloylpiperazin-1-yl)azetidin-1-yl)- 6-(2-(oxetan-3-yl)-2,8-diazaspiro[4.5]decan- 8-yl)-2-(trifluoromethyl)nicotinonitrile |
| 3-049 | 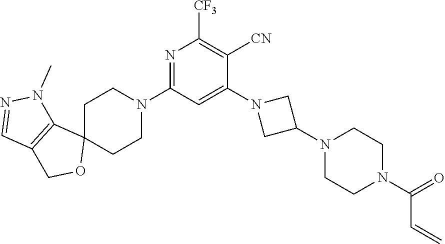 [SMILES: C=CC(=O)N1CCN(C2CN(c3cc(N4CCC5(CC4)OCc4cnn(C)c45)nc(C(F)(F)F)c3C#N)C2)CC1] | 4-(3-(4-acryloylpiperazin-1-yl)azetidin-1-yl)- 6-(1-methyl-1,4-dihydrospiro[furo[3,4- c]pyrazole-6,4′-piperidin]-1′-yl)-2- (trifluoromethyl)nicotinonitrile |
| 3-050 | 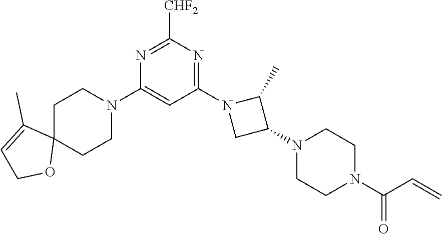 [SMILES: C=CC(=O)N1CCN([C@@H]2CN(c3cc(N4CCC5(CC4)OCC=C5C)nc(C(F)F)n3)[C@@H]2C)CC1] | 1-(4-((2R,3R)-1-(2-(Difluoromethyl)-6-(4- methyl1-oxa-8-azaspiro[4.5]dec3-en-8- yl)pyrimidin-4-yl)-2-methylazetidin-3- yl)piperazin-1-yl)prop2en-1-one |
| 3-051 | 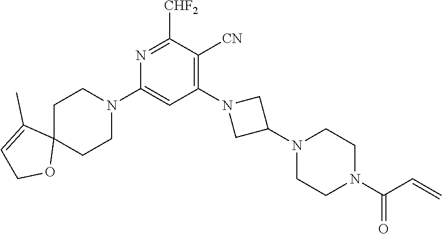 [SMILES: C=CC(=O)N1CCN(C2CN(c3cc(N4CCC5(CC4)OCC=C5C)nc(C(F)F)c3C#N)C2)CC1] | 4-(3-(4-acryloylpiperazin-1-yl)azetidin-1-yl)- 6-(4-methyl-1-oxa-8-azaspiro[4.5]dec-3-en- 8-yl)-2-(trifluoromethyl)nicotinonitrile |
| 3-052 | 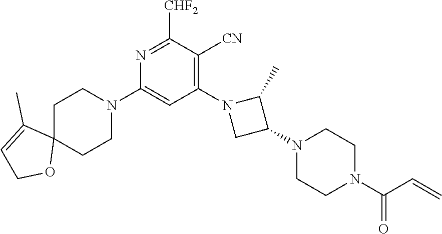 [SMILES: C=CC(=O)N1CCN([C@@H]2CN(c3cc(N4CCC5(CC4)OCC=C5C)nc(C(F)F)c3C#N)[C@@H]2C)CC1] | 4-((2R,3R)-3-(4-acryloylpiperazin-1-yl)-2- methylazetidin-1-yl)-6-(4-methyl-1-oxa-8- azaspiro[4.5]dec-3-en-8-yl)-2- (trifluoromethyl)nicotinonitrile |
| 3-053 | 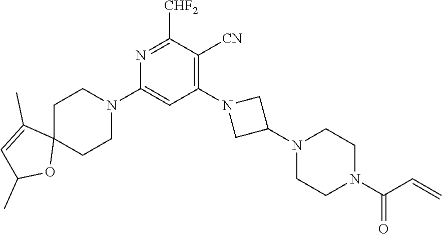 [SMILES: C=CC(=O)N1CCN(C2CN(c3cc(N4CCC5(CC4)OC(C)C=C5C)nc(C(F)F)c3C#N)C2)CC1] | 4-(3-(4-acryloylpiperazin-1-yl)azetidin-1-yl)- 6-(2,4-dimethyl-1-oxa-8-azaspiro[4.5]dec-3- en-8-yl)-2-(trifluoromethyl)nicotinonitrile |
| 3-054-1 | 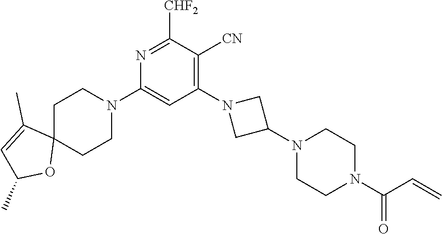 [SMILES: C=CC(=O)N1CCN(C2CN(c3cc(N4CCC5(CC4)O[C@H](C)C=C5C)nc(C(F)F)c3C#N)C2)CC1] | (R)-4-(3-(4-acryloylpiperazin-1-yl)azetidin-1- yl)-6-(2,4-dimethyl-1-oxa-8- azaspiro[4.5]dec-3-en-8-yl)-2- (trifluoromethyl)nicotinonitrile |
| 3-054-2 | 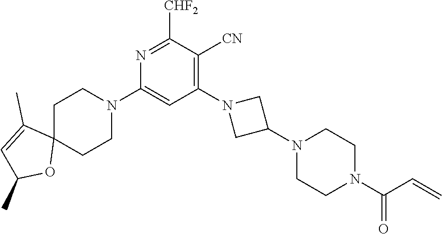 [SMILES: C=CC(=O)N1CCN(C2CN(c3cc(N4CCC5(CC4)O[C@@H](C)C=C5C)nc(C(F)F)c3C#N)C2)CC1] | (S)-4-(3-(4-acryloylpiperazin-1-yl)azetidin-1- yl)-6-(2,4-dimethyl-1-oxa-8- azaspiro[4.5]dec-3-en-8-yl)-2- (trifluoromethyl)nicotinonitrile |
| 3-055 | 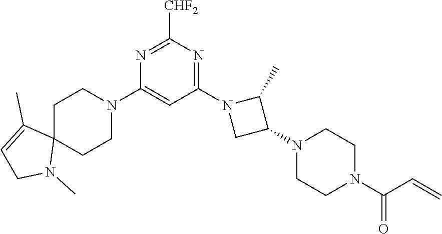 [SMILES: C=CC(=O)N1CCN([C@@H]2CN(c3cc(N4CCC5(CC4)C(C)=CCN5C)nc(C(F)F)n3)[C@@H]2C)CC1] | 1-(4-((2R,3R)-1-(6-(1,4-dimethyl-1,8- diazaspiro[4.5]dec-3-en-8-yl)-2- (trifluoromethyl)pyrimidin-4-yl)-2- methylazetidin-3-yl)piperazin-1-yl)prop-2- en1-one |
| 3-057 | 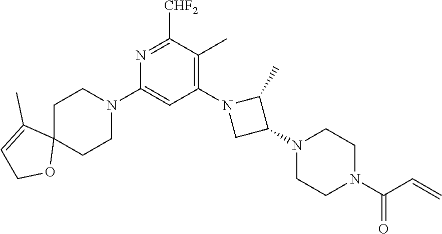 [SMILES: C=CC(=O)N1CCN([C@@H]2CN(c3cc(N4CCC5(CC4)OCC=C5C)nc(C(F)F)c3C)[C@@H]2C)CC1] | 1-(4-((2R,3R)-1-(2-(difluoromethyl)-3- methyl-6-(4-methyl-1-oxa-8- azaspiro[4.5]dec-3-en-8-yl)pyridin-4-yl)-2- methylazetidin-3-yl)piperazin-1-yl)prop-2- en-1-one |
| 3-058 | 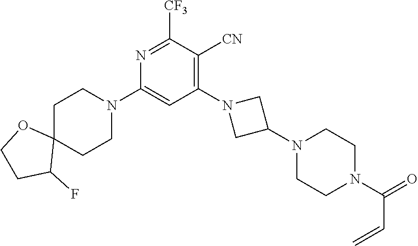 [SMILES: C=CC(=O)N1CCN(C2CN(c3cc(N4CCC5(CC4)OCCC5F)nc(C(F)(F)F)c3C#N)C2)CC1] | 4-(3-(4-acryloylpiperazin-1-yl)azetidin-1-yl)- 6-(4-fluoro-1-oxa-8-azaspiro[4.5]decan-8-yl)- 2-(trifluoromethyl)nicotinonitrile |
| 3-059 | 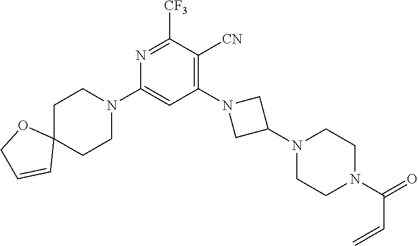 [SMILES: C=CC(=O)N1CCN(C2CN(c3cc(N4CCC5(C=CCO5)CC4)nc(C(F)(F)F)c3C#N)C2)CC1] | 4-(3-(4-acryloylpiperazin-1-yl)azetidin-1-yl)- 6-(1-oxa-8-azaspiro[4.5]dec-3-en-8-yl)-2- (trifluoromethyl)nicotinonitrile |
| 3-060 | 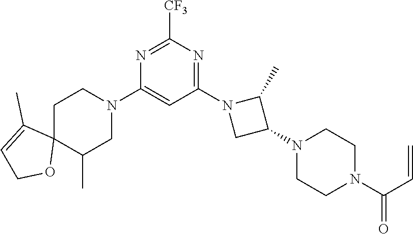 [SMILES: C=CC(=O)N1CCN([C@@H]2CN(c3cc(N4CCC5(OCC=C5C)C(C)C4)nc(C(F)(F)F)n3)[C@@H]2C)CC1] | 1-(4-((2R,3R)-1-(6-(4,6-dimethyl-1-oxa-8- azaspiro[4.5]dec-3-en-8-yl)-2- (trifluoromethyl)pyrimidin-4-yl)-2- methylazetidin-3-yl)piperazin-1-yl)prop-2- en-1-one |
| 3-061 | 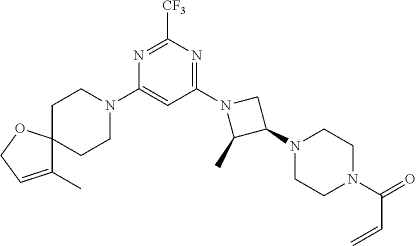 [SMILES: C=CC(=O)N1CCN([C@@H]2CN(c3cc(N4CCC5(CC4)OCC=C5C)nc(C(F)(F)F)n3)[C@@H]2C)CC1] | 1-(4-((2R,3R)-2-methyl-1-(6-(4-methyl-1- oxa-8-azaspiro[4.5]dec-3-en-8-yl)-2- (trifluoromethyl)pyrimidin-4-yl)azetidin-3- yl)piperazin-1-yl)prop-2-en-1-one |
| 3-062-1 | 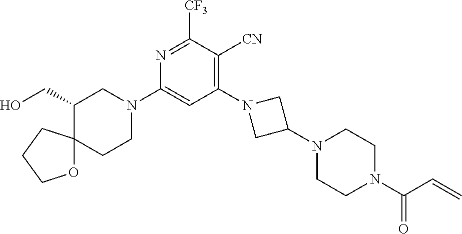 [SMILES: C=CC(=O)N1CCN(C2CN(c3cc(N4CCC5(CCCO5)[C@H](CO)C4)nc(C(F)(F)F)c3C#N)C2)CC1] | 4-(3-(4-acryloylpiperazin-1-yl)azetidin-1-yl)- 6-((6S)-6-(hydroxymethyl)-1-oxa-8- azaspiro[4.5]decan-8-yl)-2- (trifluoromethyl)nicotinonitrile |
| 3-060-2 | 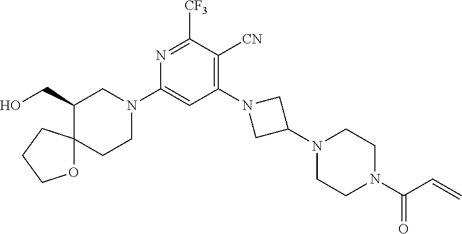 [SMILES: C=CC(=O)N1CCN(C2CN(c3cc(N4CCC5(CCCO5)[C@@H](CO)C4)nc(C(F)(F)F)c3C#N)C2)CC1] | 4-(3-(4-acryloylpiperazin-1-yl)azetidin-1-yl)- 6-((6R)-6-(hydroxymethyl)-1-oxa-8- azaspiro[4.5]decan-8-yl)-2- (trifluoromethyl)nicotinonitrile |
| 3-063 | 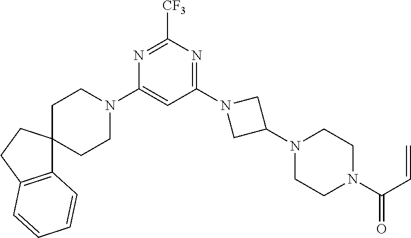 [SMILES: C=CC(=O)N1CCN(C2CN(c3cc(N4CCC5(CCc6ccccc65)CC4)nc(C(F)(F)F)n3)C2)CC1] | 1-(4-(1-(6-(2,3-dihydrospiro[indene-1,4′- piperidin]-1′-yl)-2- (trifluoromethyl)pyrimidin-4-yl)azetidin-3- yl)piperazin-1-yl)prop-2-en-1-one |
| 3-064 | 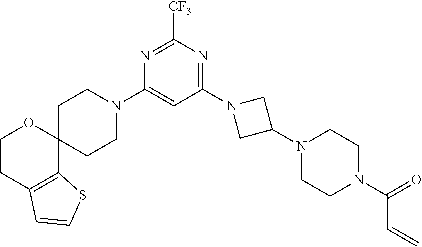 [SMILES: C=CC(=O)N1CCN(C2CN(c3cc(N4CCC5(CC4)OCCc4ccsc45)nc(C(F)(F)F)n3)C2)CC1] | 1-(4-(1-(6-(4′,5′-dihydrospiro[piperidine-4.7′- thieno[2,3-c]pyran]-1-yl)-2- (trifluoromethyl)pyrimidin-4-yl)azetidin-3- yl)piperazin-1-yl)prop-2-en-1-one |
| 3-065 | 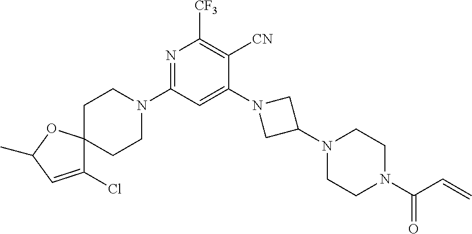 [SMILES: C=CC(=O)N1CCN(C2CN(c3cc(N4CCC5(CC4)OC(C)C=C5Cl)nc(C(F)(F)F)c3C#N)C2)CC1] | 4-(3-(4-acryloylpiperazin-1-yl)azetidin-1-yl)- 6-(4-chloro-2-methyl-1-oxa-8- azaspiro[4.5]dec-3-en-8-yl)-2- (trifluoromethyl)nicotinonitrile |
| 3-066 | 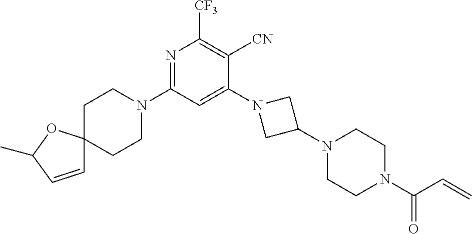 [SMILES: C=CC(=O)N1CCN(C2CN(c3cc(N4CCC5(C=CC(C)O5)CC4)nc(C(F)(F)F)c3C#N)C2)CC1] | 4-(3-(4-acryloylpiperazin-1-yl)azetidin-1-yl)- 6-(2-methyl-1-oxa-8-azaspiro[4.5]dec-3-en- 8-yl)-2-(trifluoromethyl)nicotinonitrile |
| 3-067-1 | 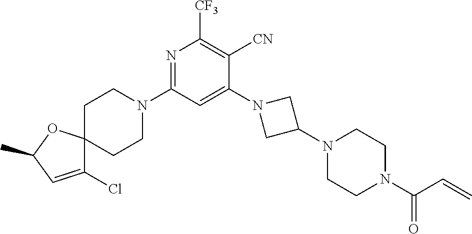 [SMILES: C=CC(=O)N1CCN(C2CN(c3cc(N4CCC5(CC4)O[C@H](C)C=C5Cl)nc(C(F)(F)F)c3C#N)C2)CC1] | (R)-4-(3-(4-acryloylpiperazin-1-yl)azetidin-1- yl)-6-(4-chloro-2-methyl-1-oxa-8- azaspiro[4.5]dec-3-en-8-yl)-2- (trifluoromethyl)nicotinonitrile |
| 3-067-2 | 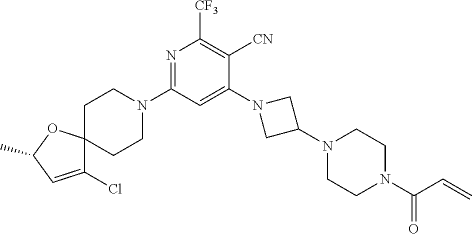 [SMILES: C=CC(=O)N1CCN(C2CN(c3cc(N4CCC5(CC4)O[C@@H](C)C=C5Cl)nc(C(F)(F)F)c3C#N)C2)CC1] | (S)-4-(3-(4-acryloylpiperazin-1-yl)azetidin-1- yl)-6-(4-chloro-2-methyl-1-oxa-8- azaspiro[4.5]dec-3-en-8-yl)-2- (trifluoromethyl)nicotinonitrile |
| 3-068-1 | 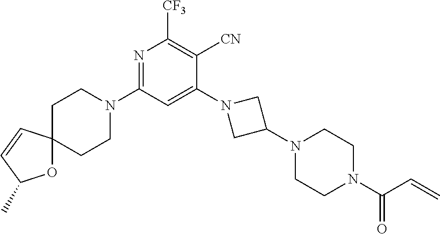 [SMILES: C=CC(=O)N1CCN(C2CN(c3cc(N4CCC5(C=C[C@@H](C)O5)CC4)nc(C(F)(F)F)c3C#N)C2)CC1] | (R)-4-(3-(4-acryloylpiperazin-1-yl)azetidin-1- yl)-6-(2-methyl-1-oxa-8-azaspiro[4.5]dec-3- en-8-yl)-2-(trifluoromethyl)nicotinonitrile |
| 3-068-2 | 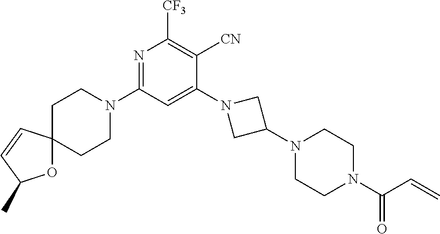 [SMILES: C=CC(=O)N1CCN(C2CN(c3cc(N4CCC5(C=C[C@H](C)O5)CC4)nc(C(F)(F)F)c3C#N)C2)CC1] | (S)-4-(3-(4-acryloylpiperazin-1-yl)azetidin-1- yl)-6-(2-methyl-1-oxa-8-azaspiro[4.5]dec-3- en-8-yl)-2-(trifluoromethyl)nicotinonitrile |
| 3-069 | 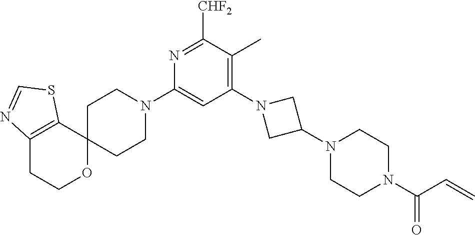 [SMILES: C=CC(=O)N1CCN(C2CN(c3cc(N4CCC5(CC4)OCCc4ncsc45)nc(C(F)F)c3C)C2)CC1] | 1-(4-(1-(2-(difluoromethyl)-6-(6′ [symbol] 7′- dihydrospiro[piperidine-4,4′-pyrano[4.3- d]thiazol]-1-yl)-3-methylpyridin-4- yl)azetidin-3-yl)piperazin-1-yl)prop-2-en-1- one |
| 3-070 | 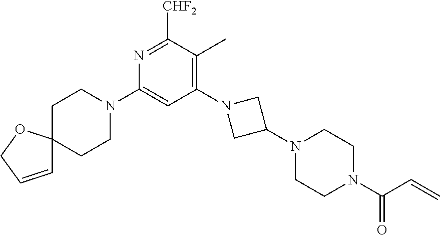 [SMILES: C=CC(=O)N1CCN(C2CN(c3cc(N4CCC5(C=CCO5)CC4)nc(C(F)F)c3C)C2)CC1] | 1-(4-(1-(2-(difluoromethyl)-3-methyl-6-(1- oxa-8-azaspiro[4.5]dec-3-en-8-yl)pyridin-4- yl)azetidin-3-yl)piperazin-1-yl)prop-2-en-1- one |
| 3-071 | 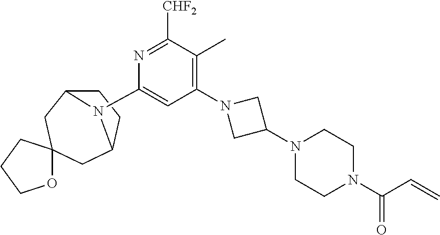 [SMILES: C=CC(=O)N1CCN(C2CN(c3cc(N4C5CCC4CC4(CCCO4)C5)nc(C(F)F)c3C)C2)CC1] | 1-(4-(1-(2-(difluoromethyl)-6-(dihydro-3′H- 8-azaspiro[bicyclo[3.2.1]octane-3 [symbol] 2′-furan]- 8-yl)-3-methylpyridin-4-yl)azetidin-3- yl)piperazin-1-yl)prop-2-en-1-one |
| 3-072 | 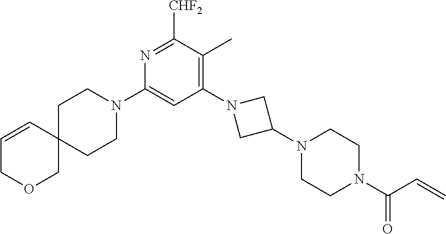 [SMILES: C=CC(=O)N1CCN(C2CN(c3cc(N4CCC5(C=CCOC5)CC4)nc(C(F)F)c3C)C2)CC1] | 1-(4-(1-(2-(difluoromethyl)-3-methyl-6-(2- oxa-9-azaspiro[5.5]undec-4-en-9-yl)pyridin- 4-yl)azetidin-3-yl)piperazin-1-yl)prop-2-en- 1-one |
| 3-073 | 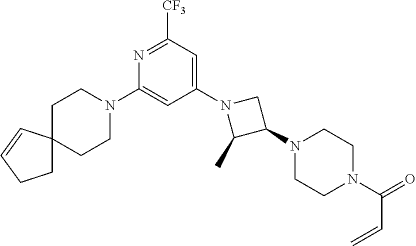 [SMILES: C=CC(=O)N1CCN([C@@H]2CN(c3cc(N4CCC5(C=CCC5)CC4)nc(C(F)(F)F)c3)[C@@H]2C)CC1] | 1-(4-((2R,3R)-1-(6-(8-azaspiro[4.5]dec-1-en- 8-yl)-2-(trifluoromethyl)pyrimidin-4-yl)-2- methylazetidin-3-yl)piperazin-1-yl)prop-2- en-1-one |
| 3-074 | 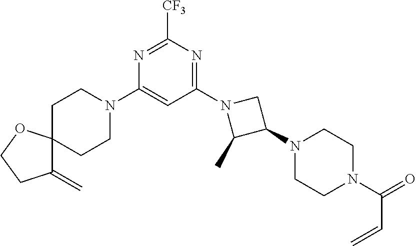 [SMILES: C=CC(=O)N1CCN([C@@H]2CN(c3cc(N4CCC5(CC4)OCCC5=C)nc(C(F)(F)F)n3)[C@@H]2C)CC1] | 1-(4-((2R,3R)-2-methyl-1-(6-(4-methylene-1- oxa-8-azaspiro[4.5]decan8-yl)-2- (trifluoromethyl)pyrimidin-4-yl)azetidin-3- yl)piperazin-1-yl)prop-2-en-1-one |
| 3-075 | 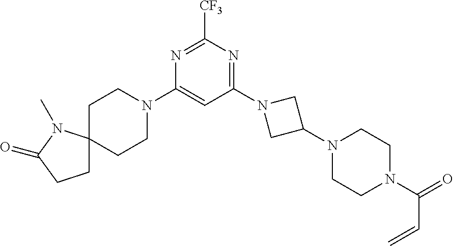 [SMILES: C=CC(=O)N1CCN(C2CN(c3cc(N4CCC5(CCC(=O)N5C)CC4)nc(C(F)(F)F)n3)C2)CC1] | 4-(3-(4-acryloylpiperazin-1-yl)azetidin-1-yl)- 6-(1-methyl-2-oxo-1,8-diazaspiro[4.5]decan- 8-yl)-2-(trifluoromethyl)nicotinonitrile |
| 3-076 | 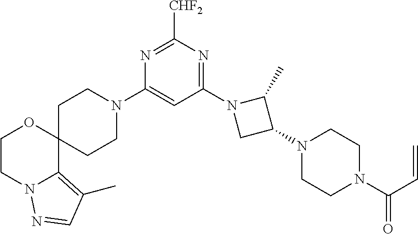 [SMILES: C=CC(=O)N1CCN([C@@H]2CN(c3cc(N4CCC5(CC4)OCCn4ncc(C)c45)nc(C(F)F)n3)[C@@H]2C)CC1] | 1-(4-((2R,3R)-2-methyl-1-(6-(3′-methyl-6′,7′- dihydrospiro[piperidine-4 [symbol] 4′-pyrazolo[5,1- c][1,4]oxazin]-1-yl)-2- (trifluoromethyl)pyrimidin-4-yl)azetidin-3- yl)piperazin-1-yl)prop-2-en-1-one |
| 3-077 | 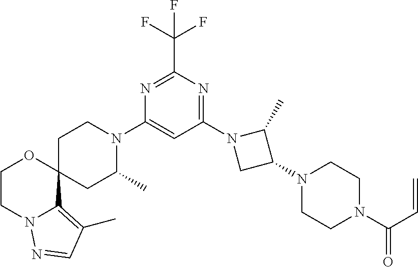 [SMILES: C=CC(=O)N1CCN([C@@H]2CN(c3cc(N4CC[C@]5(C[C@H]4C)OCCn4ncc(C)c45)nc(C(F)(F)F)n3)[C@@H]2C)CC1] | 1-(4-((2R,3R)-1-(6-((2R,4R)-2 [symbol] 3′-dimethyl- 6′,7′-dihydro-1H-spiro[piperidine-4,4′- pyrazolo[5 [symbol] 1-c][1,4]oxazin]-1-yl)-2- (trifluoromethyl)-4-pyrimidinyl)-2-methyl-3- azetidinyl)-1-piperazinyl)-2-propen-1-one |
| 3-078 | 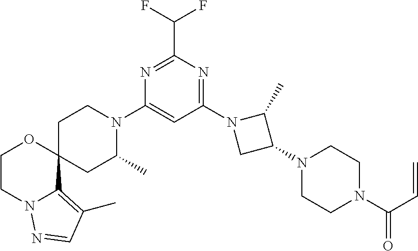 [SMILES: C=CC(=O)N1CCN([C@@H]2CN(c3cc(N4CC[C@]5(C[C@H]4C)OCCn4ncc(C)c45)nc(C(F)F)n3)[C@@H]2C)CC1] | 1-(4-((2R,3R)-1-(2-(difluoromethyl)-6- ((2R,4R)-2,3′-dimethyl-6′,7′-dihydro-1H- spiro[piperidine-4,4′-pyrazolo[5,1- c][1,4]oxazin]-1-yl)-4-pyrimidinyl)-2- methyl-3-azetidinyl)-1-piperazinyl)-2- propen-1-one |
| 3-079 | 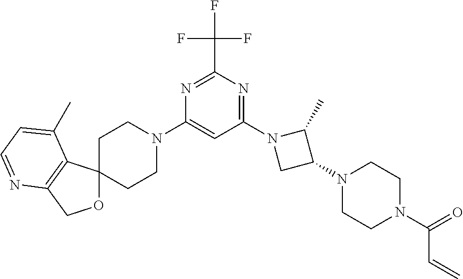 [SMILES: C=CC(=O)N1CCN([C@@H]2CN(c3cc(N4CCC5(CC4)OCc4nccc(C)c45)nc(C(F)(F)F)n3)[C@@H]2C)CC1] | 1-(4-((2R,3R)-2-methyl-1-(6-(4-methyl- 1′H,7H-spiro[furo[3,4-b]pyridine-5,4′- piperidin]-1′-yl)-2-(trifluoromethyl)-4- pyrimidinyl)-3-azetidinyl)-1-piperazinyl)-2- propen-1-one |
| 3-080 | 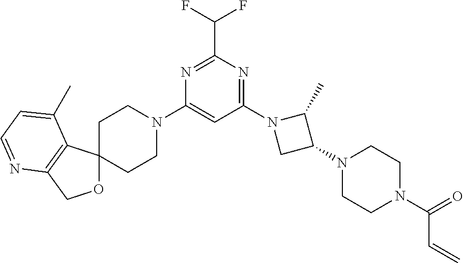 [SMILES: C=CC(=O)N1CCN([C@@H]2CN(c3cc(N4CCC5(CC4)OCc4nccc(C)c45)nc(C(F)F)n3)[C@@H]2C)CC1] | 1-(4-((2R,3R)-1-(2-(difluoromethyl)-6-(4- methyl-7H-spiro[furo[3,4-b]pyridine-5,4′- piperidin]-1′-yl)pyrimidin-4-yl)-2- methylazetidin-3-yl)piperazin-1-yl)prop-2- en-1-one |
| 3-081 | 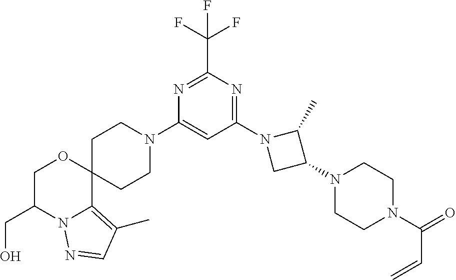 [SMILES: C=CC(=O)N1CCN([C@@H]2CN(c3cc(N4CCC5(CC4)OCC(CO)n4ncc(C)c45)nc(C(F)(F)F)n3)[C@@H]2C)CC1] | 1-(4-((2R,3R)-1-(6-(7′-(hydroxymethyl)-3′- methyl-6′,7′-dihydrospiro[piperidine-4,4′- pyrazolo[5,1-c][1,4]oxazin]-1-yl)-2- (trifluoromethyl)pyrimidin-4-yl)-2- methylazetidin-3-yl)piperazin-1-yl)prop-2- en-1-one |
| 3-082 | 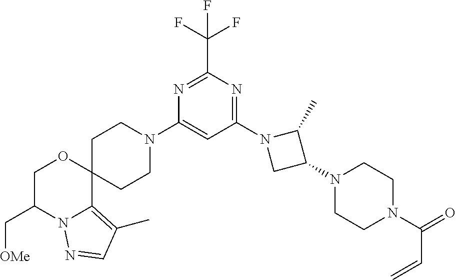 [SMILES: C=CC(=O)N1CCN([C@@H]2CN(c3cc(N4CCC5(CC4)OCC(COC)n4ncc(C)c45)nc(C(F)(F)F)n3)[C@@H]2C)CC1] | 1-(4-((2R,3R)-1-(6-(7′-(methoxymethyl)-3′- methyl-6′,7′-dihydrospiro[piperidine-4,4′- pyrazolo[5,1-c][1,4]oxazin]-1-yl)-2- (trifluoromethyl)pyrimidin-4-yl)-2- methylazetidin-3-yl)piperazin-1-yl)prop-2- en-1-one |
| 3-083 | 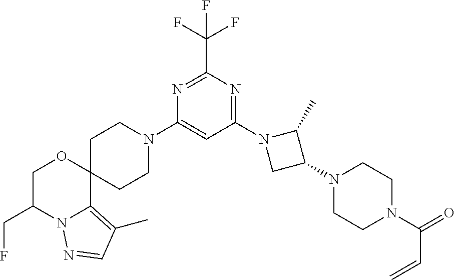 [SMILES: C=CC(=O)N1CCN([C@@H]2CN(c3cc(N4CCC5(CC4)OCC(CF)n4ncc(C)c45)nc(C(F)(F)F)n3)[C@@H]2C)CC1] | 1-(4-((2R,3R)-1-(6-(7′-(fluoromethyl)-3′- methyl-6′,7′-dihydrospiro[piperidine-4,4′- pyrazolo[5,1-c][1,4]oxazin]-1-yl)-2- (trifluoromethyl)pyrimidin-4-yl)-2- methylazetidin-3-yl)piperazin-1-yl)prop-2- en-1-one |
| 3-084 | 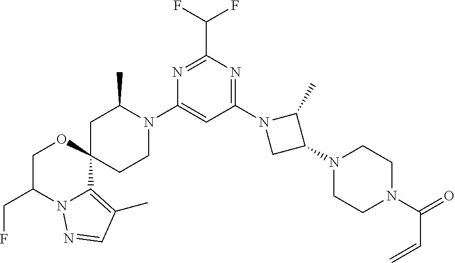 | 1-(4-((2R,3R)-1-(2-(difluoromethyl)-6- ((2R,4R)-7′-(fluoromethyl)-2,3′-dimethyl- 6′,7′-dihydrospiro[piperidine-4,4′- pyrazolo[5,1-c][1,4]oxazin]-1-yl)pyrimidin- 4-yl)-2-methylazetidin-3-yl)piperazin-1- yl)prop-2-en-1-one |
| 3-085 | 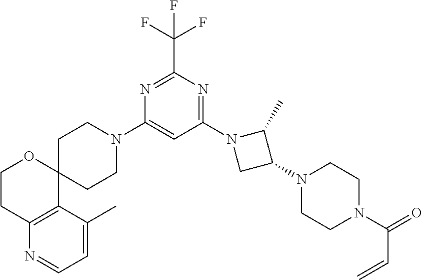 [SMILES: C=CC(=O)N1CCN([C@@H]2CN(c3cc(N4CCC5(CC4)OCCc4nccc(C)c45)nc(C(F)(F)F)n3)[C@@H]2C)CC1] | 1-(4-((2R,3R)-2-methyl-1-(6-(4′-methyl-7′,8′- dihydrospiro[piperidine-4,5′-pyrano[4,3- b]pyridin]-1-yl)-2- (trifluoromethyl)pyrimidin-4-yl)azetidin-3- yl)piperazin-1-yl)prop-2-en-1-one |
| 3-086 | 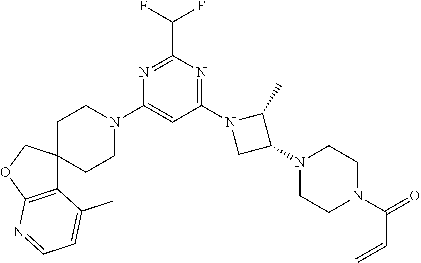 [SMILES: C=CC(=O)N1CCN([C@@H]2CN(c3cc(N4CCC5(CC4)COc4nccc(C)c45)nc(C(F)F)n3)[C@@H]2C)CC1] | 1-(4-((2R,3R)-1-(2-(difluoromethyl)-6-(4- methyl-2H-spiro[furo[2,3-b]pyridine-3,4′- piperidin]-1′-yl)pyrimidin-4-yl)-2- methylazetidin-3-yl)piperazin-1-yl)prop-2- en-1-one |
| 3-087-1 | 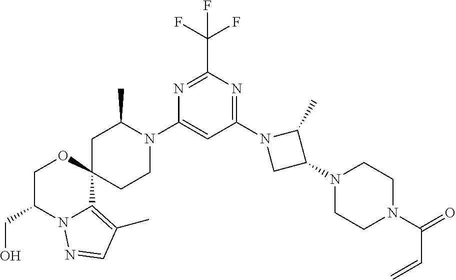 [SMILES: C=CC(=O)N1CCN([C@@H]2CN(c3cc(N4CC[C@]5(C[C@H]4C)OC[C@@H](CO)n4ncc(C)c45)nc(C(F)(F)F)n3)[C@@H]2C)CC1] | 1-(4-((2R,3R)-1-(6-((2R,4R,7′R)-7′- (hydroxymethyl)-2,3′-dimethyl-6′,7′- dihydrospiro[piperidine-4,4′-pyrazolo[5,1- c][1,4]oxazin]-1-yl)-2- (trifluoromethyl)pyrimidin-4-yl)-2- methylazetidin-3-yl)piperazin-1-yl)prop-2- en-1-one |
| 3-087-2 | 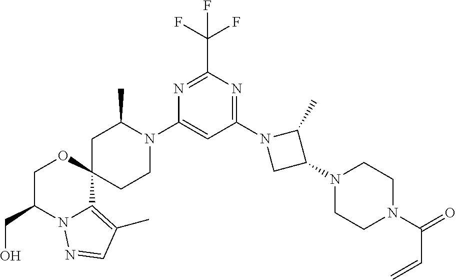 [SMILES: C=CC(=O)N1CCN([C@@H]2CN(c3cc(N4CC[C@]5(C[C@H]4C)OC[C@H](CO)n4ncc(C)c45)nc(C(F)(F)F)n3)[C@@H]2C)CC1] | 1-(4-((2R,3R)-1-(6-((2R,4R,7′S)-7′- (hydroxymethyl)-2,3′-dimethyl-6′,7′- dihydrospiro[piperidine-4,4′-pyrazolo[5,1- c][1,4]oxazin]-1-yl)-2- (trifluoromethyl)pyrimidin-4-yl)-2- methylazetidin-3-yl)piperazin-1-yl)prop-2- en-1-one |
| 3-088-1 | 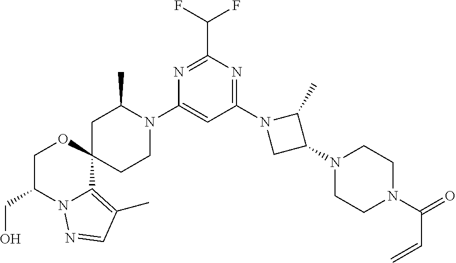 [SMILES: C=CC(=O)N1CCN([C@@H]2CN(c3cc(N4CC[C@]5(C[C@H]4C)OC[C@@H](CO)n4ncc(C)c45)nc(C(F)F)n3)[C@@H]2C)CC1] | 1-(4-((2R,3R)-1-(2-(difluoromethyl)-6- ((2R,4R,7′R)-7′-(hydroxymethyl)-2,3′- dimethyl-6′,7′-dihydrospiro[piperidine-4,4′- pyrazolo[5,1-c][1,4]oxazin]-1-yl)pyrimidin- 4-yl)-2-methylazetidin-3-yl)piperazin-1- yl)prop-2-en-1-one |
| 3-088-2 | 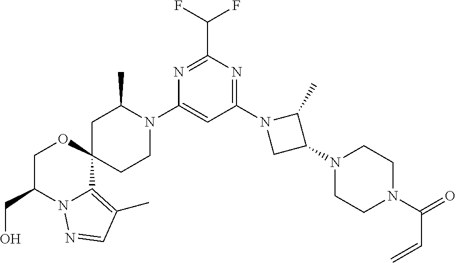 [SMILES: C=CC(=O)N1CCN([C@@H]2CN(c3cc(N4CC[C@]5(C[C@H]4C)OC[C@H](CO)n4ncc(C)c45)nc(C(F)F)n3)[C@@H]2C)CC1] | 1-(4-((2R,3R)-1-(2-(difluoromethyl)-6- ((2R,4R,7′S)-7′-(hydroxymethyl)-2,3′- dimethyl-6′,7′-dihydrospiro[piperidine-4,4′- pyrazolo[5,1-c][1,4]oxazin]-1-yl)pyrimidin- 4-yl)-2-methylazetidin-3-yl)piperazin-1- yl)prop-2-en-1-one |
| 3-089-1 | 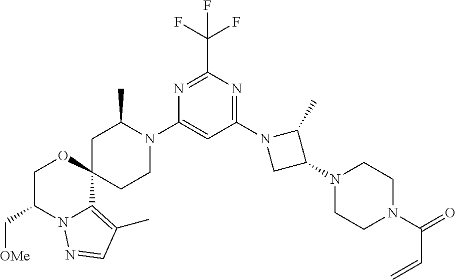 [SMILES: C=CC(=O)N1CCN([C@@H]2CN(c3cc(N4CC[C@]5(C[C@H]4C)OC[C@@H](COC)n4ncc(C)c45)nc(C(F)(F)F)n3)[C@@H]2C)CC1] | 1-(4-((2R,3R)-1-(6-((2R,4R,7′S)-7′- (methoxymethyl)-2,3′-dimethyl-6′,7′- dihydrospiro[piperidine-4,4′-pyrazolo[5,1- c][1,4]oxazin]-1-yl)-2- (trifluoromethyl)pyrimidin-4-yl)-2- methylazetidin-3-yl)piperazin-1-yl)prop-2- en-1-one |
| 3-090-1 | 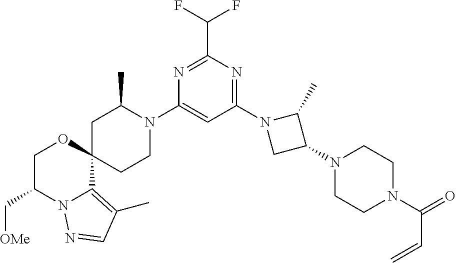 [SMILES: C=CC(=O)N1CCN([C@@H]2CN(c3cc(N4CC[C@]5(C[C@H]4C)OC[C@@H](COC)n4ncc(C)c45)nc(C(F)F)n3)[C@@H]2C)CC1] | 1-(4-((2R,3R)-1-(2-(difluoromethyl)-6- ((2R,4R,7′R)-7′-(methoxymethyl)-2,3′- dimethyl-6′,7′-dihydrospiro[piperidine-4,4′- pyrazolo[5,1-c][1,4]oxazin]-1-yl)pyrimidin- 4-yl)-2-methylazetidin-3-yl)piperazin-1- yl)prop-2-en-1-one |
| 3-091-1 | 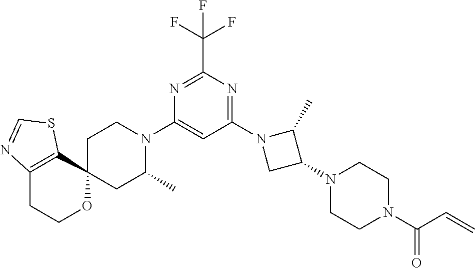 [SMILES: C=CC(=O)N1CCN([C@@H]2CN(c3cc(N4CC[C@]5(C[C@H]4C)OCCc4ncsc45)nc(C(F)(F)F)n3)[C@@H]2C)CC1] | 1-(4-((2R,3R)-2-methyl-1-(6-((2R,4R)-2- methyl-6′,7′-dihydrospiro[piperidine-4,4′- pyrano[4,3-d]thiazol]-1-yl)-2- (trifluoromethyl)pyrimidin-4-yl)azetidin-3- yl)piperazin-1-yl)prop-2-en-1-one |
| 3-091-2 | 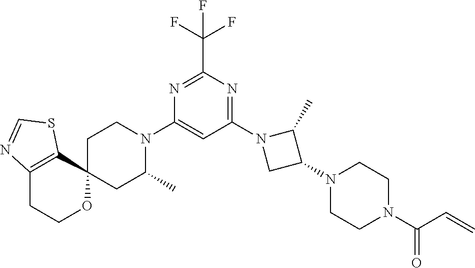 [SMILES: C=CC(=O)N1CCN([C@@H]2CN(c3cc(N4CC[C@]5(C[C@H]4C)OCCc4ncsc45)nc(C(F)(F)F)n3)[C@@H]2C)CC1] | 1-(4-((2R,3R)-2-methyl-1-(6-((2S,4S)-2- methyl-6′,7′-dihydrospiro[piperidine-4,4′- pyrano[4,3-d]thiazol]-1-yl)-2- (trifluoromethyl)pyrimidin-4-yl)azetidin-3- yl)piperazin-1-yl)prop-2-en-1-one |
| 3-092 | 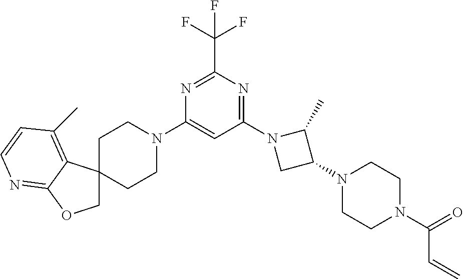 [SMILES: C=CC(=O)N1CCN([C@@H]2CN(c3cc(N4CCC5(CC4)COc4nccc(C)c45)nc(C(F)(F)F)n3)[C@@H]2C)CC1] | 1-(4-((2R,3R)-2-methyl-1-(6-(4-methyl-1′H- spiro[furo[2,3-b]pyridine-3,4′-piperidin]-1′- yl)-2-(trifluoromethyl)-4-pyrimidinyl)-3- azetidinyl)-1-piperazinyl)-2-propen-1-one |
| 3-093 | 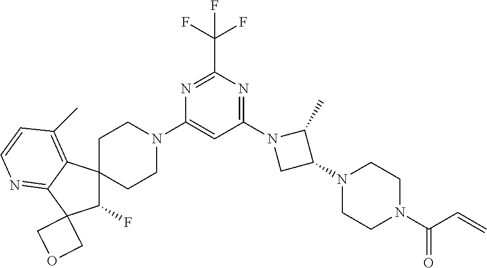 [SMILES: C=CC(=O)N1CCN([C@@H]2CN(c3cc(N4CCC5(CC4)c4c(C)ccnc4C4(COC4)[C@H]5F)nc(C(F)(F)F)n3)[C@@H]2C)CC1] | 1-(4-((2R,3R)-1-(6-((6′R)-6′-fluoro-4′-methyl- 1″H-dispiro[oxetane-3,7′- cyclopenta[b]pyridine-5′,4″-piperidin]-1″-yl)- 2-(trifluoromethyl)-4-pyrimidinyl)-2-methyl- 3-azetidinyl)-1-piperazinyl)-2-propen-1-one |
| 3-094 | 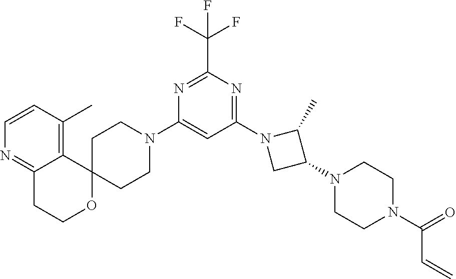 [SMILES: C=CC(=O)N1CCN([C@@H]2CN(c3cc(N4CCC5(CC4)OCCc4nccc(C)c45)nc(C(F)(F)F)n3)[C@@H]2C)CC1] | 1-(4-((2R,3R)-2-methyl-1-(6-(4′-methyl-7′,8′- dihydro-1H-spiro[piperidine-4,5′-pyrano[4,3- b]pyridin]-1-yl)-2-(trifluoromethyl)-4- pyrimidinyl)-3-azetidinyl)-1-piperazinyl)-2- propen-1-one |
| 3-095 | 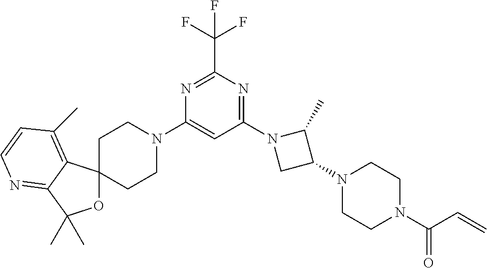 [SMILES: C=CC(=O)N1CCN([C@@H]2CN(c3cc(N4CCC5(CC4)OC(C)(C)c4nccc(C)c45)nc(C(F)(F)F)n3)[C@@H]2C)CC1] | 1-(4-((2R,3R)-2-methyl-1-(2- (trifluoromethyl)-6-(4,7,7-trimethyl-1′H-7H- spiro[3,4-b]pyridine-5,4′-piperidin]-1′- yl)-4-pyrimidinyl)-3-azetidinyl)-1- piperazinyl)-2-propen-1-one |
| 3-096 | 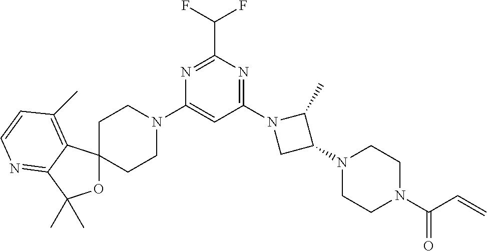 [SMILES: C=CC(=O)N1CCN([C@@H]2CN(c3cc(N4CCC5(CC4)OC(C)(C)c4nccc(C)c45)nc(C(F)F)n3)[C@@H]2C)CC1] | 1-(4-((2R,3R)-1-(2-(difluoromethyl)-6-(4,7,7- trimethyl-1′H,7H-spiro[furo[3,4-b]pyridine- 5,4′-piperidin]-1′-yl)-4-pyrimidinyl)-2- methyl-3-azetidinyl)-1-piperazinyl)-2- propen-1-one |
| 3-097 | 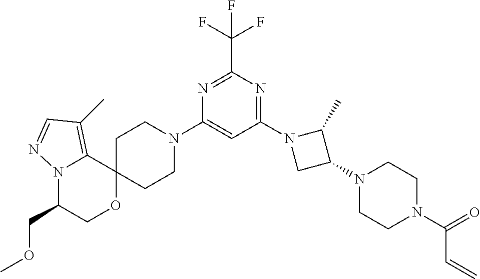 [SMILES: C=CC(=O)N1CCN([C@@H]2CN(c3cc(N4CCC5(CC4)OC[C@@H](COC)n4ncc(C)c45)nc(C(F)(F)F)n3)[C@@H]2C)CC1] | 1-(4-((2R,3R)-1-(6-((7′R)-7′- (methoxymethyl)-3′-methyl-6′,7′-dihydro-1H- spiro [symbol] piperidine-4,4′-pyrazolo[5,1- c][1,4]oxazin]-1-yl)-2-(trifluoromethyl)-4- pyrimidinyl)-2-methyl-3-azetidinyl)-1- piperazinyl)-2-propen-1-one |
| 3-098 | 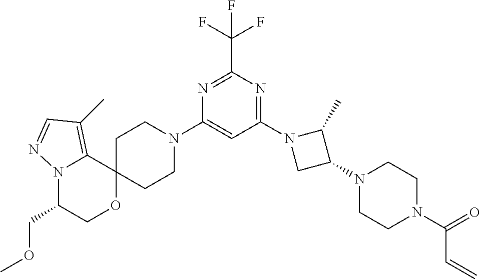 [SMILES: C=CC(=O)N1CCN([C@@H]2CN(c3cc(N4CCC5(CC4)OC[C@H](COC)n4ncc(C)c45)nc(C(F)(F)F)n3)[C@@H]2C)CC1] | 1-(4-((2R,3R)-1-(6-((7′S)-7′- (methoxymethyl)-3′-methyl-6′,7′-dihydro-1H- spiro[piperidine-4,4′-pyrazolo[5,1- c][1,4]oxazin]-1-yl)-2-(trifluoromethyl)-4- pyrimidinyl)-2-methyl-3-azetidinyl)-1- piperazinyl)-2-propen-1-one |
| 3-099 | 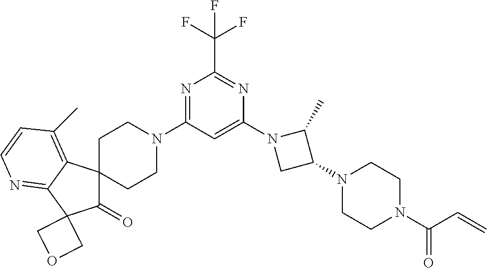 [SMILES: C=CC(=O)N1CCN([C@@H]2CN(c3cc(N4CCC5(CC4)C(=O)C4(COC4)c4nccc(C)c45)nc(C(F)(F)F)n3)[C@@H]2C)CC1] | 4′-methyl-1″-(6-((2R,3R)-2-methyl-3-(4-(2- propenyl)-1-piperazinyl)-1-azetidinyl)-2- (trifluoromethyl)-4- pyrimidinyl)dispiro[oxetane-3,7′- cyclopenta[b]pyridine-5′,4″-piperidin]-6′-one |
| 3-100 | 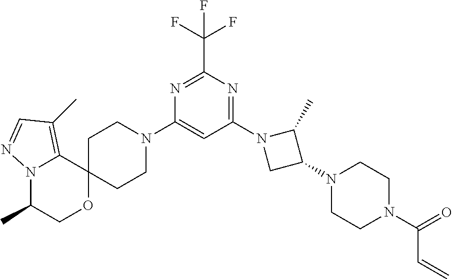 [SMILES: C=CC(=O)N1CCN([C@@H]2CN(c3cc(N4CCC5(CC4)OC[C@@H](C)n4ncc(C)c45)nc(C(F)(F)F)n3)[C@@H]2C)CC1] | 1-(4-((2R,3R)-1-(6-((7′R)-3′,7′-dimethyl-6′ [symbol] 7′- dihydro-1H-spiro[piperidine-4,4′- pyrazolo[5,1-c][1,4]oxazin]-1-yl)-2- (trifluoromethyl)-4-pyrimidinyl)-2-methyl-3- azetidinyl)-1-piperazinyl)-2-propen-1-one |
| 3-101 | 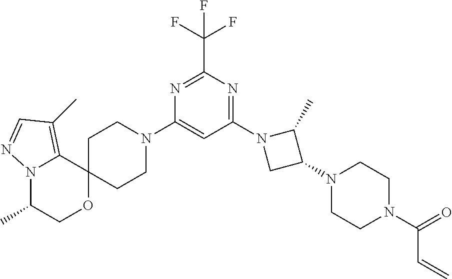 [SMILES: C=CC(=O)N1CCN([C@@H]2CN(c3cc(N4CCC5(CC4)OC[C@H](C)n4ncc(C)c45)nc(C(F)(F)F)n3)[C@@H]2C)CC1] | 1-(4-((2R,3R)-1-(6-((7′S)-3′,7′-dimethyl-6′ [symbol] 7′- dihydro-1H-spiro[piperidine-4,4′- pyrazolo[5,1-c][1,4]oxazin]-1-yl)-2- (trifluoromethyl)-4-pyrimidinyl)-2-methyl-3- azetidinyl)-1-piperazinyl)-2-propen-1-one |
| 3-102 | 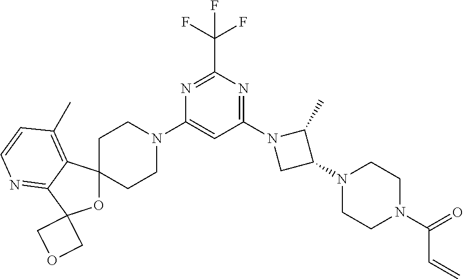 [SMILES: C=CC(=O)N1CCN([C@@H]2CN(c3cc(N4CCC5(CC4)OC4(COC4)c4nccc(C)c45)nc(C(F)(F)F)n3)[C@@H]2C)CC1] | 1-(4-((2R,3R)-2-methyl-1-(6-(4′-methyl-1″H- dispiro[oxetane-3,7′-furo[3,4-b]pyridine- 5′,4″-piperidin]-1″-yl)-2-(trifluoromethyl)-4- pyrimidinyl)-3-azetidinyl)-1-piperazinyl)-2- propen-1-one |
| 3-103 | 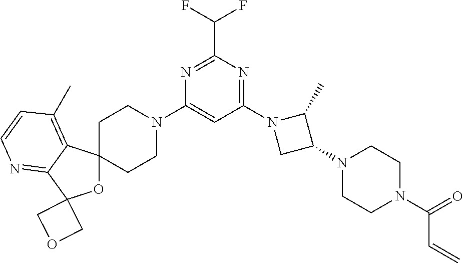 [SMILES: C=CC(=O)N1CCN([C@@H]2CN(c3cc(N4CCC5(CC4)OC4(COC4)c4nccc(C)c45)nc(C(F)F)n3)[C@@H]2C)CC1] | 1-(4-((2R,3R)-1-(2-(difluoromethyl)-6-(4′- methyl-1″H-dispiro[oxetane-3,7′-furo[3,4- b]pyridine-5′,4″-piperidin]-1″-yl)-4- pyrimidinyl)-2-methyl-3-azetidinyl)-1- piperazinyl)-2-propen-1-one |
| 3-104 | 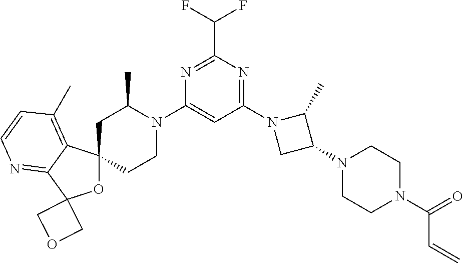 [SMILES: C=CC(=O)N1CCN([C@@H]2CN(c3cc(N4CC[C@]5(C[C@H]4C)OC4(COC4)c4nccc(C)c45)nc(C(F)F)n3)[C@@H]2C)CC1] | 1-(4-((2R,3R)-1-(6-((2″R,5′R)-2″,4′-dimethyl- 1″H-dispiro[oxetane-3,7′-furo[3,4-b]pyridine- 5′,4″-piperidin]-1″-yl)-2-(trifluoromethyl)-4- pyrimidinyl)-2-methyl-3-azetidinyl)-1- piperazinyl)-2-propen-1-one |

[symbol]

indicates data missing or illegible when filed

In some cases, the compound of Formula (I) or Formula (II) is selected from the list of compounds in Table C. In some cases, the compound of Formulae (I) and (II) is compound selected from compound 3-001 through compound 3-104 (as shown in Table C).

In some cases, the disclosure provides a compound of Formula (I) or Formula (II), or a pharmaceutically acceptable salts of any of the foregoing, wherein Y′ is C₃₋₇cycloalkyl or C₄₋₇cycloalkenyl. In some cases, the compound of Formula (I) or Formula (II) is Compound 3-022, 3-023, 3-036, 3-037, 3-038, 3-063, 3-073, 3-093, or 3-099, or a pharmaceutically acceptable salt of any of the foregoing.

In some cases, the disclosure provides a compound of Formula (I) or Formula (II), or a pharmaceutically acceptable salts of any of the foregoing, wherein Y′ is oxetanyl or dioxanyl. In some cases, the compound of Formulae (I) or (II) is Compound 3-019 or 3-024, or a pharmaceutically acceptable salt of any of the foregoing.

In some cases, the disclosure provides a compound of Formula (I) or Formula (II), or a pharmaceutically acceptable salts of any of the foregoing, wherein Y′ is tetrahydrofuranyl and p is 0. In some cases, the compound of Formulae (I) or (II) is Compound 3-025, 3-026, 3-039, 3-062-1, 3-062-2, or 3-071, or a pharmaceutically acceptable salt of any of the foregoing.

In some cases, the disclosure provides a compound of Formula (I) or Formula (II), or a pharmaceutically acceptable salts of any of the foregoing, wherein Y′ is tetrahydrofuranyl and each p is CH₂, oxo, halo, CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, C₀₋₄alkylene-C₃₋₇cycloalkyl, C₀₋₄alkylene-heterocycloalkyl having 3-7 total ring atoms and 1, 2, or 3 heteroatoms selected from N, O, and S, C₀₋₄alkylene-C₆₋₁₀aryl, C₀₋₄alkylene-heteroaryl having 5-7 total ring atoms and 1, 2, or 3 heteroatoms selected from N, O, and S. In some cases, the compound of Formula (I) or Formula (II) is Compound 3-029, 3-030, 3-031, 3-044, 3-045, 3-058, or 3-074, or a pharmaceutically acceptable salt of any of the foregoing.

In some cases, the disclosure provides a compound of Formula (I) or Formula (II), or a pharmaceutically acceptable salts of any of the foregoing, wherein Y′ is tetrahydrofuranyl and two vicinal R⁷ together with the atoms to which they are attached form optionally substituted fused-C₃₋₇cycloalkyl or optionally substituted fused-C₆₋₁₀aryl. In some cases, the cycloalkyl and aryl is unsubstituted or substituted with 1-3 substituents, and each substituent independently is halo, CN, oxo, or C₁₋₃alkyl. In some cases, the compound of Formula (I) or Formula (II) is Compound 3-015, 3-018, 3-027, 3-028, 3-040, 3-041, or 3-042, or a pharmaceutically acceptable salt of any of the foregoing.

In some cases, the disclosure provides a compound of Formula (I) or Formula (II), or a pharmaceutically acceptable salts of any of the foregoing, wherein Y′ is tetrahydrofuranyl and two vicinal R⁷ together with the atoms to which they are attached form optionally substituted fused-heteroaryl having 5-7 total ring atoms and 1-3 heteroatoms independently selected from N. O, and S. In some cases, the heteroaryl is unsubstituted or substituted with 1-3 substituents, and each substituent independently is halo, CN, oxo, or C₁₋₃alkyl. In some cases, the compound of Formula (I) or Formula (II) is Compound 3-001, 3-043, 3-046, 3-047, 3-049, 3-079, 3-080, 3-086, 3-092, 3-095, 3-096, 3-102, 3-103, or 3-104, or a pharmaceutically acceptable salt of any of the foregoing.

In some cases, the disclosure provides a compound of Formula (I) or Formula (II), or a pharmaceutically acceptable salts of any of the foregoing, wherein Y′ is tetrahydropyranyl and p is 0. In some cases, the compound of Formula (I) or Formula (II) is Compound 3-035, or a pharmaceutically acceptable salt thereof.

In some cases, the disclosure provides a compound of Formula (I) or Formula (II), or a pharmaceutically acceptable salts of any of the foregoing, wherein Y′ is tetrahydropyranyl and two vicinal R², together with the atoms to which they are attached, form a fused thiazolyl ring, a fused thiophenyl ring, or a fused pyridyl ring. In some cases, the compound of Formula (I) or Formula (II) is 3-002, 3-064, 3-069, 3-085, 3-091, 3-092, or 3-094, or a pharmaceutically acceptable salt of any of the foregoing.

In some cases, the disclosure provides a compound of Formula (I) or Formula (II), or a pharmaceutically acceptable salts of any of the foregoing, wherein Y′ is morpholinyl; p is 2 or 3; and two vicinal R⁷, together with the atoms to which they are attached, form optionally substituted fused-pyrazolyl. In some cases, the pyrazolyl is unsubstituted or substituted with 1-3 substituents, and each substituent independently is halo, CN, oxo, or C₁₋₃alkyl. In some cases, the compound of Formula (I) or Formula (II) is Compound 3-076, 3-077, 3-078, 3-081, 3-082, 3-083, 3-084, 3-087-1, 3-087-2, 3-088-1, 3-088-2, 3-089-1, 3-089-2, 3-090-1, 3-090-2, 3-097, 3-098, 3-100, or 3-101, or a pharmaceutically acceptable salt of any of the foregoing.

In some cases, the disclosure provides a compound of Formula (I) or Formula (II), or a pharmaceutically acceptable salts of any of the foregoing, wherein Y′ is thiazolidinyl; p is 2, 3 or 4; and at least one R⁷ is oxo. In some cases, the compound of Formula (I) or Formula (II) is Compound 3-032, 3-033, or 3-034, or a pharmaceutically acceptable salt of any of the foregoing.

In some cases, the disclosure provides a compound of Formula (I) or Formula (II), or a pharmaceutically acceptable salts of any of the foregoing, wherein Y′ is pyrrolidinyl. In some cases, the compound of Formula (I) or Formula (II) is Compound 3-004, 3-013, 3-014, 3-016, 3-020, 3-048, or 3-075, or a pharmaceutically acceptable salt of any of the foregoing

In some cases, the disclosure provides a compound of Formula (I) or Formula (II), or a pharmaceutically acceptable salts of any of the foregoing, wherein Y′ is optionally substituted dihyropyrrolyl, isoxazolyl, dihydrofuranyl or dihydropyranyl. In some cases, the compound of Formula (I) or Formula (II) is Compound 3-003, 3-005, 3-006, 3-007, 3-008, 3-009, 3-010, 3-011, 3-012, 3-017, 3-021, 3-050, 3-051, 3-052, 3-053, 3-054-1, 3-054-2, 3-055, 3-057, 3-059, 3-060, 3-061, 3-065, 3-066, 3-067-1, 3-067-2, 3-068-1, 3-068-2, 3-070, or 3-072, or a pharmaceutically acceptable salt of any of the foregoing.

In some cases, the compound of Formula (I) or Formula (II) is selected from the group consisting of Compound 3-001, 3-002, 3-003, 3-004, 3-005, 3-008, 3-009, 3-010, 3-011, 3-014, 3-015, 3-016, 3-017, 3-046, 3-049, 3-050, 3-051, 3-052, 3-053, 3-054-1, 3-054-2, 3-057, 3-058, 3-059, 3-060, 3-061, 3-065, 3-066, 3-067-1, 3-067-2, 3-069, 3-074, 3-075, 3-076, 3-077, 3-078, 3-079, 3-080, 3-081, 3-082, 3-083, 3-084, 3-085, 3-087-1, 3-087-2, 3-088-1, 3-088-2, 3-089-1, 3-089-2, 3-090-1, 3-091-1, or 3-091-2, or a pharmaceutically acceptable salt of any of the foregoing.

In some cases, the compound of Formula (I) or Formula (II) is selected from the group consisting of Compound 3-001, 3-002, 3-004, 3-005, 3-008, 3-014, 3-046, 3-051, 3-052, 3-053, 3-054-1, 3-054-2, 3-057, 3-059, 3-060, 3-061, 3-074, 3-076, 3-077, 3-078, 3-079, 3-080, 3-081, 3-082, 3-083, 3-084, 3-085, 3-087-1, 3-087-2, 3-088-1, 3-088-2, 3-089-1, 3-089-2, 3-090-1, or 3-091-1, or a pharmaceutically acceptable salt of any of the foregoing.

In some cases, the compound of Formula (I) or Formula (II) is selected from the group consisting of Compound 3-004, 3-005, 3-051, 3-052, 3-053, 3-054-1, 3-059, 3-061, 3-076, 3-077, 3-078, 3-079, 3-081, 3-082, 3-083, 3-084, 3-085, 3-087-1, 3-087-2, 3-088-1, 3-088-2, 3-089-1, 3-089-2, 3-090-1, or 3-091-1, or a pharmaceutically acceptable salt of any of the foregoing.

In some cases, the compound of Formula (I) or Formula (II) is selected from the group consisting of Compound 3-076, 3-077, 3-078, 3-079, 3-081, 3-082, 3-083, 3-085, 3-087-1, 3-087-2, 3-088-2, 3-089-1, 3-089-2, or 3-091-1, or a pharmaceutically acceptable salt of any of the foregoing.

In some cases, the compound of Formula (I) or Formula (II) is selected from the group consisting of Compound 3-092, 3-093, 3-094, 3-095, 3-096, 3-097, 3-098, 3-099, 3-100, 3-101, 3-102, 3-103, or 3-104, or a pharmaceutically acceptable salt of any of the foregoing.

In some cases, the compound of Formula (I) or Formula (II) is selected from the list of compounds in Table D, below. If the stereochemistry of a structure or a portion of a structure in Table D is not explicitly shown (e.g., such as with dashed or bold lines), then the structure or portion of structure is either achiral or interpreted as being any of the possible stereoisomers of the structure or portion of the structure. In cases in which the stereochemistry of the structure or portion of the structure in Table D is explicitly shown, a single stereoisomer of the structure or portion of a structure is represented, with the understanding that the stereochemistry of the structure or portion of the structure shown in Table D has been arbitrarily assigned.

TABLE D

| Compound | | |
|---|---|---|
| No. | Chemical Structure | Name |
| 3-001 | 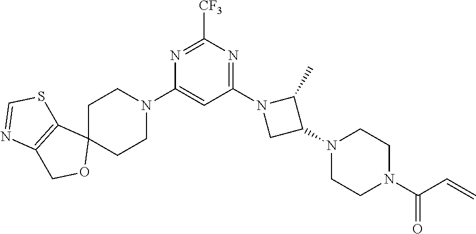 | 1-(4-((2R,3R)-1-(6-(4H-spiro[furo[3,4- d]thiazole-6,4′-piperidin]-1′-yl)-2- (trifluoromethyl)pyrimidin-4-yl)-2- methylazetidin-3-yl)piperazin-1- yl)prop-2-en-1-one |
| 3-002 | 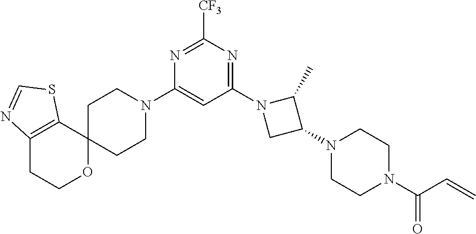 [SMILES: C=CC(=O)N1CCN([C@@H]2CN(c3cc(N4CCC5(CC4)OCCc4ncsc45)nc(C(F)(F)F)n3)[C@@H]2C)CC1] | 1-(4-((2R,3R)-1-(6-(6′,7′- dihydrospiro[piperidine-4,4′-pyrano[4.3- d]thiazol]-1-yl)-2- (trifluoromethyl)pyrimidin-4-yl)-2- methylazetidin-3-yl)piperazin-1- yl)prop-2-en-1-one |
| 3-052 | 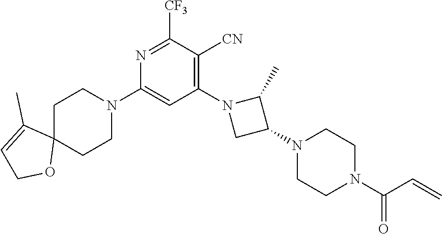 [SMILES: C=CC(=O)N1CCN([C@@H]2CN(c3cc(N4CCC5(CC4)OCC=C5C)nc(C(F)(F)F)c3C#N)[C@@H]2C)CC1] | 4-((2R,3R)-3-(4-acryloylpiperazin-1-yl)- 2-methylazetidin-1-yl)-6-(4-methyl-1- oxa-8-azaspiro[4.5]dec-3-en-8-yl)-2- (trifluoromethyl)nicotinonitrile |
| 3-076 | 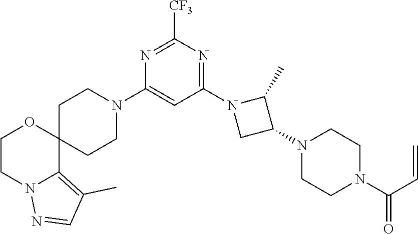 [SMILES: C=CC(=O)N1CCN([C@@H]2CN(c3cc(N4CCC5(CC4)OCCn4ncc(C)c45)nc(C(F)(F)F)n3)[C@@H]2C)CC1] | 1-(4-((2R,3R)-2-methyl-1-(6-(3′-methyl- 6′,7′-dihydrospiro[piperidine-4,4′- pyrazolo[5,1-c][1,4]oxazin]-1-yl)-2- (trifluoromethyl)pyrimidin-4- yl)azetidin-3-yl)piperazin-1-yl)prop-2- en-1-one |
| 3-077 | 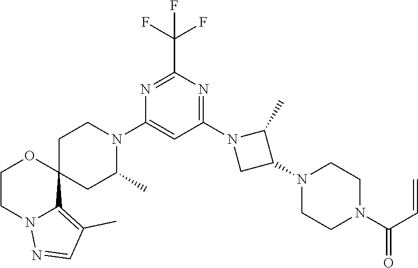 | 1-(4-((2R,3R)-1-(6-((2R.4R)-2,3′- dimethyl-6′,7′-dihydro-1H- spiro[piperidine-4,4′-pyrazolo[5,1- c][1.4]oxazin]-1-yl)-2-(trifluoromethyl)- 4-pyrimidinyl)-2-methyl-3-azetidinyl)- 1-piperazinyl)-2-propen-1-one |
| 3-078 | 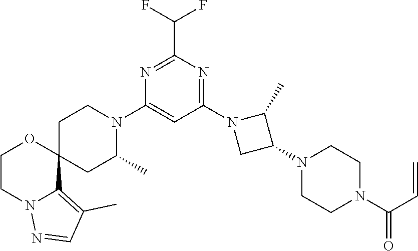 [SMILES: C=CC(=O)N1CCN([C@@H]2CN(c3cc(N4CC[C@]5(C[C@H]4C)OCCn4ncc(C)c45)nc(C(F)F)n3)[C@@H]2C)CC1] | 1-(4-((2R,3R)-1-(2-(difluoromethyl)-6- ((2R,4R)-2,3′-dimethyl-6′,7′-dihydro- 1H-spiro[piperidine-4,4′-pyrazolo[5,1- c][1,4]oxazin]-1-yl)-4-pyrimidinyl)-2- methyl-3-azetidinyl)-1-piperazinyl)-2- propen-1-one |
| 3-079 | 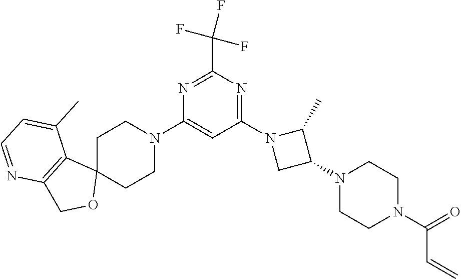 [SMILES: C=CC(=O)N1CCN([C@@H]2CN(c3cc(N4CCC5(CC4)OCc4nccc(C)c45)nc(C(F)(F)F)n3)[C@@H]2C)CC1] | 1-(4-((2R,3R)-2-methyl-1-(6-(4-methyl- 1′H,7H-spiro[furo[3,4-b]pyridine-5,4′- piperidin]-1′-yl)-2-(trifluoromethyl)-4- pyrimidinyl)-3-azetidinyl)-1- piperazinyl)-2-propen-1-one |
| 3-085 | 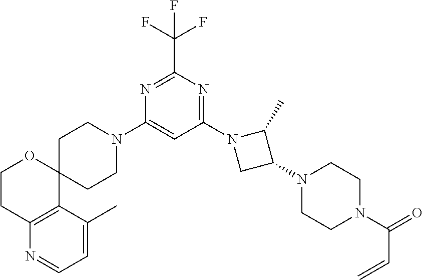 [SMILES: C=CC(=O)N1CCN([C@@H]2CN(c3cc(N4CCC5(CC4)OCCc4nccc(C)c45)nc(C(F)(F)F)n3)[C@@H]2C)CC1] | 1-(4-((2R,3R)-2-methyl-1-(6-(4′-methyl- 7′,8′-dihydrospiro[piperidine-4,5′- pyrano[4,3-b]pyridin]-1-yl)-2- (trifluoromethyl)pyrimidin-4- yl)azetidin-3-yl)piperazin-1-yl)prop-2- en-1-one |
| 3-089-1 | 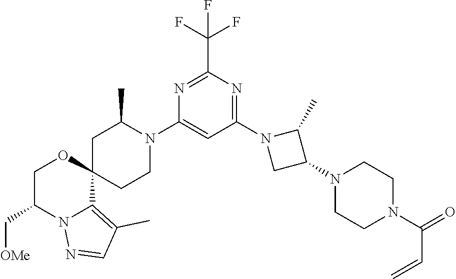 [SMILES: C=CC(=O)N1CCN([C@@H]2CN(c3cc(N4CC[C@]5(C[C@H]4C)OC[C@@H](COC)n4ncc(C)c45)nc(C(F)(F)F)n3)[C@@H]2C)CC1] | 1-(4-((2R,3R)-1-(6-((2R,4R,7′S)-7′- (methoxymethyl)-2,3′-dimethyl-6′,7′- dihydrospiro[piperidine-4,4′- pyrazolo[5,1-c][1,4]oxazin]-1-yl)-2- (trifluoromethyl)pyrimidin-4-yl)-2- methylazetidin-3-yl)piperazin-1- yl)prop-2-en-1-one |
| 3-090-1 | 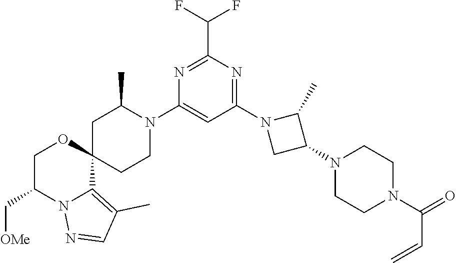 [SMILES: C=CC(=O)N1CCN([C@@H]2CN(c3cc(N4CC[C@]5(C[C@H]4C)OC[C@@H](COC)n4ncc(C)c45)nc(C(F)F)n3)[C@@H]2C)CC1] | 1-(4-((2R,3R)-1-(2-(difluoromethyl)-6- ((2R,4R,7′R)-7′-(methoxymethyl)-2,3′- dimethyl-6′,7′-dihydrospiro[piperidine- 4,4′-pyrazolo[5,1-c][1,4]oxazin]-1- yl)pyrimidin-4-yl)-2-methylazetidin-3- yl)piperazin-1-yl)prop-2-en-1-one |
| 3-091-1 | 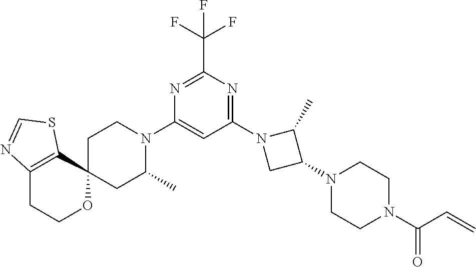 [SMILES: C=CC(=O)N1CCN([C@@H]2CN(c3cc(N4CC[C@]5(C[C@H]4C)OCCc4ncsc45)nc(C(F)(F)F)n3)[C@@H]2C)CC1] | 1-(4-((2R,3R)-2-methyl-1-(6-((2R,4R)- 2-methyl-6′,7′-dihydrospiro[piperidine- 4,4′-pyrano[4,3-d]thiazol]-1-yl)-2- (trifluoromethyl)pyrimidin-4- yl)azetidin-3-yl)piperazin-1-yl)prop-2- en-1-one |

In some cases, the compound of Formula (I) or Formula (II) is Compound 3-001, or a pharmaceutically acceptable salt thereof. In some cases, the compound of Formula (I) or Formula (II) is Compound 3-002, or a pharmaceutically acceptable salt thereof. In some cases, the compound of Formula (I) or Formula (II) is Compound 3-052, or a pharmaceutically acceptable salt thereof. In some cases, the compound of Formula (I) or Formula (II) is Compound 3-076, or a pharmaceutically acceptable salt thereof. In some cases, the compound of Formula (I) or Formula (II) is Compound 3-077, or a pharmaceutically acceptable salt thereof. In some cases, the compound Formula (I) or Formula (II) is Compound 3-078, or a pharmaceutically acceptable salt thereof. In some cases, the compound of Formula (I) or Formula (II) is Compound 3-079, or a pharmaceutically acceptable salt thereof. In some cases, the compound of Formula (I) or Formula (II) is Compound 3-085, or a pharmaceutically acceptable salt thereof. In some cases, the compound of Formula (I) or Formula (II) is Compound 3-089-1, or a pharmaceutically acceptable salt thereof. In some cases, the compound of Formula (I) or Formula (II) is Compound 3-090-1, or a pharmaceutically acceptable salt thereof. In some cases, the compound of Formula (I) or Formula (II) is Compound 3-091-1, or a pharmaceutically acceptable salt thereof.

Unless otherwise indicated, the depictions of partial structures do not represent any particular orientation of the partial structure. For example, compounds of Formulae (I) and (II) having

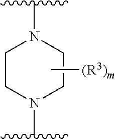

as

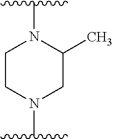 [SMILES: *N1CCN(*)C(C)C1]

include compounds of Formula (I) depicted as:

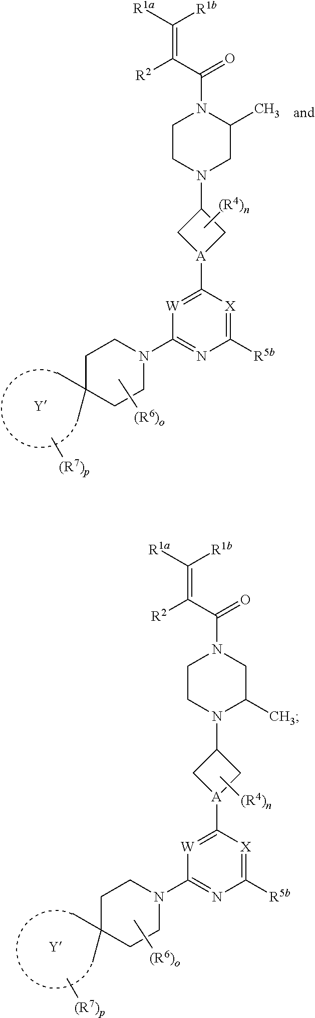

and compounds of Formula (II) depicted as:

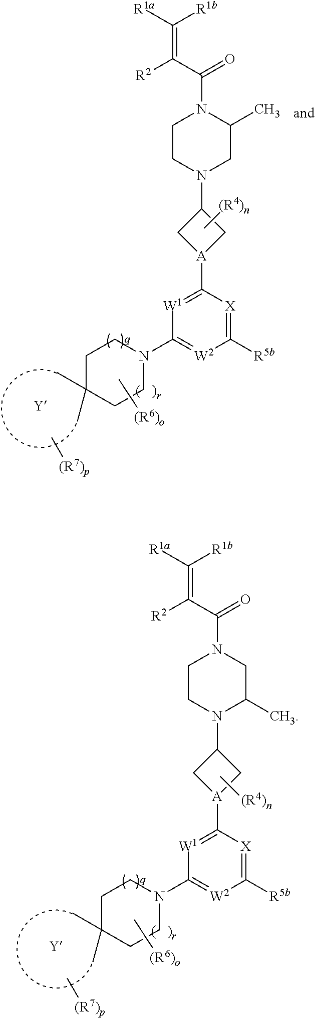

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the present disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated, unless otherwise noted. For example,

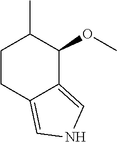

represents

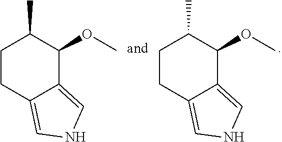

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole. In certain instances, a bond drawn with a wavy line may be used to indicate that both stereoisomers are encompassed. This is not to be confused with a wavy line drawn perpendicular to a bond which indicates the point of attachment of a group to the rest of the molecule.

The term “stereoisomer” or “stereoisomerically pure” compound, as used herein, refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example,

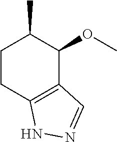

represents

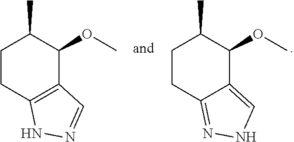

Similarly, for example, the chemical name (4R,5R)-4-methoxy-5-methyl-4,5,6,7-tetrahydro-1H-indazole represents (4R,5R)-4-methoxy-5-methyl-4,5,6,7-tetrahydro-1H-indazole and (4R,5R)-4-methoxy-5-methyl-4,5,6,7-tetrahydro-2H-indazole. Accordingly, the scope of the instant disclosure is to be understood to encompass all tautomeric forms of the compounds disclosed herein.

### Isotopically-Labeled Compounds

Further, the scope of the present disclosure includes all pharmaceutically acceptable isotopically-labelled compounds of the compounds disclosed herein, such as the compounds of Formula (I), Formula (IA), Formula (IB), Formula (II), Formula (IIA), Formula (IIB), and Formula (IIC), wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of Formula (I) and Formula (II), for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. As such, the term “deuterated” refers to the substitution of one or more hydrogen atoms with one or more deuterium atoms on a particular structure or functional group. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Procedures and Examples using an appropriate isotopically-labelled reagent in place of the non-labelled reagent previously employed.

### Biological Activity

In some cases, the compounds disclosed herein (e.g., compounds of Formula (I), Formula (IA), Formula (IB), Formula (II), Formula (IIA), Formula (IIB), Formula (IIC), compounds listed in Table A, compounds listed in Table B, compounds listed in Table C, and compounds listed in Table D, and pharmaceutically acceptable salts of any of the foregoing), have an IC₅₀ value of less than 5 μM, or less than 4 μM, or less than 3 μM, or less than 2 μM, or less than 1 μM, or less than 0.9 μM, or less than 0.8 μM, or less than 0.7 μM, or less than 0.6 μM, or less than 0.5 μM, or less than 0.4 μM, or less than 0.3 μM, or less than 0.2 μM, or less than 0.1 μM, or less than 0.09 μM, or less than 0.08 μM, or less than 0.07 μM, or less than 0.06 μM, or less than 0.05 μM, or less than 0.04 μM, or less than 0.03 μM, or less than 0.02 μM, or less than 0.01 μM in the coupled exchange assay, which is described in the “SECTION 3; Biochemical and Cellular Assays.”. In some cases, the compounds and salts disclosed herein have an IC₅₀ value of less than 1 μM. In some cases, the compounds and salts disclosed herein have an IC₅₀ value of less than 0.5 μM. In some cases, the compounds and salts disclosed herein have an IC₅₀ value of less than 0.3 μM. In some cases, the compounds and salts disclosed herein have an IC₅₀ value of less than 0.1 μM. Also provided herein are compounds of the disclosure having an IC₅₀ of less than 5 μM in the 2 h coupled exchange assay. Further provided herein are compounds of the disclosure having an IC₅₀ of less than 3 μM in the 2 h coupled exchange assay. Still further provided herein are compounds of the disclosure having an IC₅₀ of less than 1 μM in the 2 h coupled exchange assay. Still further provided herein are compounds of the disclosure, or pharmaceutically acceptable salts of the foregoing, having an IC₅₀ of less than 0.5 μM in the 2 h coupled exchange assay described herein. Also provided herein are compounds of the disclosure, or pharmaceutically acceptable salts of the foregoing, having an IC₅₀ of less than 0.1 μM in the 2 h coupled exchange assay described herein. Also provided herein are compounds of the disclosure, or pharmaceutically acceptable salts of the foregoing, having an IC₅₀ of less than 0.05 μM in the 2 h coupled exchange assay described herein. Also provided herein are compounds of the disclosure, or pharmaceutically acceptable salts of the foregoing, having an IC₅₀ of less than 0.04 μM in the 2 h coupled exchange assay described herein. Also provided herein are compounds of the disclosure, or pharmaceutically acceptable salts of the foregoing, having an IC₅₀ of less than 0.03 μM in the 2 h coupled exchange assay described herein. Also provided herein are compounds of the disclosure, or pharmaceutically acceptable salts of the foregoing, having an IC₅₀ of less than 0.02 μM in the 2 h coupled exchange assay described herein. Also provided herein are compounds of the disclosure, or pharmaceutically acceptable salts of the foregoing, having an IC₅₀ of less than 0.01 μM in the 2 h coupled exchange assay described herein.

The foregoing merely summarizes certain aspect of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, further provided herein is a pharmaceutical composition comprising a compound disclosed herein (e.g., compounds of Formula (I), Formula (IA), Formula (IB), Formula (II), Formula (IIA), Formula (IIB), Formula (IIC), compounds listed in Table A, compounds listed in Table B, compounds listed in Table C, and compounds listed in Table D, and pharmaceutically acceptable salts of any of the foregoing), in combination with one or more pharmaceutically acceptable excipients and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In some cases, the pharmaceutical composition described herein comprises a therapeutically effective amount of a compound disclosed herein, or a pharmaceutically acceptable salt thereof.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. In some cases, the pharmaceutical composition is made in the form of a dosage unit containing a particular amount of the active ingredient.

Thus, a further aspect of the disclosure is a pharmaceutical composition comprising one or more of the compounds disclosed herein, or a pharmaceutically acceptable salt thereof, and a pharmaceutically acceptable excipient. Further provided here is a compound of the disclosure, or a pharmaceutically acceptable salt thereof, or the pharmaceutical composition described herein for use as a medicament.

### Methods of Use

The compound described herein can covalently bind to cysteine-12 of the GDP-bound form of the G12C-mutant KRAS protein (“KRAS^{G12C}”). In some cases, the compounds described herein can act as potent inhibitors of KRAS^{G12C} by, for example, permanently inactivating the protein. Without intending to be bound by any particular theory, the compounds of the disclosure can, in some cases, inhibit phosphorylation of extracellular signal-regulated (“ERK”), which is a key down-stream effector of KRAS, leading to tumor regression. Besides being useful for human treatment, the compounds provided herein may be useful for veterinary treatment of companion animals, exotic animals, and farm animals, including mammals, rodents, and the like. For example, animals including horses, dogs and cats may be treated with compounds provided herein.

Another aspect of the disclosure provides methods of using the compounds disclosed herein, or pharmaceutically acceptable salts thereof, or the pharmaceutical compositions of the present disclosure to treat disease conditions, including but not limited to conditions implicated by KRAS G12C mutation (e.g., cancer). See, e.g., U.S. Pat. No. 10,519,146 B2, issued Dec. 31, 2019; specifically, the section from column 198, line 1, to column 201, line 36, which is herewith incorporated by reference.

Without wishing to be bound by any particular theory, the following is noted: sotorasib is a small molecule that—similarly to the compounds disclosed herein—specifically and irreversibly inhibits KRAS^{G12C} (see Hong et al., N. Engl. J. Med. 2020, 383, 1207, at 1208). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Section entitled “BIOLOGICAL EVALUATION” below, Canon et al., Nature 2019, 575(7781), 217; and Lanman et al., J. Med. Chem. 2020, 63, 52).

Sotorasib was evaluated in a Phase 1 dose escalation and expansion trial with 129 subjects having histologically confirmed, locally advanced or metastatic cancer with the KRAS G12C mutation identified by local molecular testing on tumor tissues, including 59 subjects with non-small cell lung cancer, 42 subjects with colorectal cancer, and 28 subjects with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma (Hong et al., 2020, at page 1212 (FIGURE A), and Supplementary Appendix (page 59 (FIGURE S5) and page 63 (FIGURE S6)).

KRAS G12C mutations occur with the alteration frequencies shown in the table below (Cerami et al., Cancer Discov. 2012, 2(5), 401; Gao et al., Science Signaling 2013, 6(269), p11). For example, the table shows that 11.6% of subjects with non-small cell lung cancer have a cancer, wherein one or more cells express KRAS G12C mutant protein. Accordingly, the compounds provided herein, which specifically and irreversibly bind to KRAS^{G12C} (see SECTION 3: Biochemical and Cellular Assays), are useful for treatment of subjects having a cancer, including, but not limited to the cancers listed in the table below.

| Cancer Type | Alteration Frequency |
|---|---|
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine Tumor | 0.4 |
| Bladder Cancer | 0.4 |
| Myelodysplastic/Myeloproliferative Neoplasms | 0.3 |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

Another aspect of the disclosure provides a compound disclosed herein (e.g., a compound of Formula (I), Formula (IA), Formula (IB), Formula (II), Formula (IIA), Formula (IIB), Formula (IIC), a compound listed in Table A, a compound listed in Table B, a compound listed in Table C, or a compound listed in Table D, or a pharmaceutically acceptable salt of any of the foregoing), or a pharmaceutically acceptable salt thereof, or a pharmaceutical composition disclosed herein, for use in treating cancer. Yet another aspect of the disclosure provides a compound disclosed herein, or a pharmaceutically acceptable salt thereof, or a pharmaceutical composition disclosed herein, for use in treating cancer, wherein one or more cells express KRAS G12C mutant protein.

Another aspect of the disclosure provides a compound disclosed herein, or a pharmaceutically acceptable salt thereof, or a pharmaceutical composition disclosed herein, in the preparation of a medicament for treating cancer. Yet another aspect of the disclosure provides a compound disclosed herein, or a pharmaceutically acceptable salt thereof, or a pharmaceutical composition described herein, in the preparation of a medicament for treating cancer, wherein one or more cells express KRAS G12C mutant protein.

A further aspect of the disclosure is a method of treating cancer in a subject in need thereof, the method comprising administering to the subject a therapeutically effective amount of a compound disclosed herein (e.g., a compound of Formula (I), Formula (IA), Formula (IB), Formula (II). Formula (IIA), Formula (IIB), Formula (IIC), a compound listed in Table A, a compound listed in Table B, a compound listed in Table C, or a compound listed in Table D, or a pharmaceutically acceptable salt of any of the foregoing), or a pharmaceutical composition described herein. Another aspect of the disclosure is a method of treating cancer in a subject in need thereof, the method comprising administering to the subject a therapeutically effective amount of a compound disclosed herein, or a pharmaceutically acceptable salt thereof, or a pharmaceutical composition of the disclosure, wherein one or more cells express KRAS G12C mutant protein. In some cases, the subject has a cancer that was determined to have one or more cells expressing the KRAS G12C mutant protein prior to administration of the compound or a pharmaceutically acceptable salt thereof.

In some cases, the cancer is metastatic. In some cases, the cancer is non-metastatic. In some cases, the cancer disclosed herein is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor. In some cases, the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor. In some cases, the cancer is non-small cell lung cancer. In some cases, the cancer is colorectal cancer. In some cases, the cancer is pancreatic cancer. In some cases, the cancer is solid tumor.

### Combination Therapy

The present disclosure also provides methods for combination therapies in which an agent known to modulate other pathways, or other components of the same pathway, or even overlapping sets of target enzymes are used in combination with a compound of the present disclosure (e.g., a compound of Formula (I), Formula (IA), Formula (IB), Formula (II), Formula (IIA), Formula (IIB), Formula (IIC), a compound listed in Table A, a compound listed in Table B, a compound listed in Table C, or a compound listed in Table D. or a pharmaceutically acceptable salt of any of the foregoing). In one aspect, such therapy includes but is not limited to the combination of one or more compounds of the disclosure with chemotherapeutic agents, therapeutic antibodies, and/or radiation treatment, to provide a synergistic or additive therapeutic effect. See, e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

The compounds of the disclosure can be administered simultaneously, separately, or sequentially with an effective amount of a second compound in any of the methods described herein. In some cases, the second compound is an ATR inhibitor, Aurora kinase A inhibitor. AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents. In some cases, the second compound is administered as a pharmaceutically acceptable salt. In some cases, the second compound is administered as a pharmaceutical composition comprising the second compound or a pharmaceutically acceptable salt thereof and a pharmaceutically acceptable excipient.

ATR inhibitors. In some cases, the compounds of the disclosure can be administered simultaneously, separately, or sequentially with an effective amount of an ATR inhibitor in any of the methods described herein. An ATR inhibitor is a compound that targets the ataxia telangiectasia mutated and Rad3-related kinase. Exemplary ATR inhibitors for use in the methods provided herein include, but are not limited to dactolisib, VE-821 (3-Amino-6-(4-(methylsulfonyl)phenyl)-N-phenylpyrazine-2-carboxamide, 3-Amino-6-[4-(methylsulfonyl)phenyl]-N-phenyl-2-pyrazinecarboxamide), Torin 2 (9-(6-amino-3-pyridinyl)-1-[3-(trifluoromethyl)phenyl]-benzo[h]-1,6-naphthyridin-2(1H)-one), ETP-46464 (α,α-dimethyl-4-[2-oxo-9-(3-quinolinyl)-2H-[1,3]oxazino[5,4132zetidinelin-1 (4H)-yl]-benzeneacetonitrile), CGK 733 (α-Phenyl-N-[2,2,2-trichloro-1-[[(4-fluoro-3-nitrophenyl)amino]thioxomethyl]amino]ethyl]benzeneacetamide), AZ20 (4-[4-[(3R)-3-Methyl-4-morpholinyl]-6-[1-(methylsulfonyl)cyclopropyl]-2-pyrimidinyl]-1H-indole), SKLB-197 ((R)-4-(2-(1H-indol-4-yl)-6-(1-methyl-1H-pyrazol-5-yl) quinazolin-4-yl)-3-methylmorpholine), elimusertib, gartisertib, elimusertib hydrochloride, ceralasertib, and schisandrin B.

Aurora Kinase A Inhibitors. In some cases, the compounds of the disclosure can be administered simultaneously, separately, or sequentially with an effective amount of an Aurora kinase A inhibitor in any of the methods described herein. Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03.7]tetradeca-1 (14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl) phthalazin-1-amine), MLN8054 (4-[[9-chloro-7-(2,6-difluorophenyl)-5H-pyrimido[5,4-d][2]benzazepin-2-yl]amino]benzoic acid). PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2 (7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino)pyrimidin-4-yl]-1,3-thiazol-2-amine), TAS-119, BI 811283, and TTP607.

AKT Inhibitors. In some cases, the compounds of the disclosure can be administered simultaneously, separately, or sequentially with an effective amount of an AKT inhibitor in any of the methods described herein. Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f][1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1 (9),5-dien-8-one), ARQ 751 (N-(3-aminopropyl)-N-[(1R)-1~ (3-anilino-7-chloro-4-oxoquinazolin-2-yl) but-3-ynyl]-3-chloro-2-fluorobenzamide), RX-0201, and LY2780301.

Arginase Inhibitors. In some cases, the compounds of the disclosure can be administered simultaneously, separately, or sequentially with an effective amount of an arginase inhibitor in any of the methods described herein. Exemplary arginase inhibitors for use in the methods provided herein include, but are not limited to, numidargistat and CB 280.

CDK 2 Inhibitors. In some cases, the compounds of the disclosure can be administered simultaneously, separately, or sequentially with an effective amount of a CDK 2 inhibitor in any of the methods described herein. The term “CDK 2” as used herein refers to cyclin dependent kinases (“CDK”) 2, which is a member of the mammalian serine/threonine protein kinases. The term “CDK 2 inhibitor” as used herein refers to a compound that is capable of negatively modulating or inhibiting all or a portion of the enzymatic activity of CDK 2. Exemplary CDK 2 inhibitors for use in the methods provided herein include, but are not limited to, flavopiridol, roscovitine, dinaciclib, milciclib, meriolin, variolin, AZDS438 (4-[2-Methyl-1-(1-methylethyl)-1H-imidazol-5-yl]-N-[4-(methylsulfonyl)phenyl]-2-pyrimidinamine), roniciclib, SNS-032 (N-[5-[[[5-(1,1-Dimethylethyl)-2-oxazolyl]methyl]thio]-2-thiazolyl]-4-piperidinecarboxamide).

CDK4/6 Inhibitors. In some cases, the compounds of the disclosure can be administered simultaneously, separately, or sequentially with an effective amount of a CDK4/6 inhibitor in any of the methods described herein. The term “CDK 4/6” as used herein refers to cyclin dependent kinases (“CDK”) 4 and 6, which are members of the mammalian serine/threonine protein kinases. The term “CDK 4/6 inhibitor” as used herein refers to a compound that is capable of negatively modulating or inhibiting all or a portion of the enzymatic activity of CDK 4 and/or 6. Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7(8H)-one, 6-(difluoromethyl)-8-[(1R,2R)-2-hydroxy-2-methylcyclopentyl-2-[[1-(methylsulfonyl)-4-piperidinyl]amino]). In some cases, the CDK4/6 inhibitor is palbociclib.

ErbB Family Inhibitors. In some cases, the compounds of the disclosure can be administered simultaneously, separately, or sequentially with an effective amount of an ErbB family inhibitor in any of the methods described herein. The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4). The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members. In some cases, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In some cases, the anti-EGFR antibody is cetuximab. In some cases, the anti-EGFR antibody is panitumumab. In some cases, the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine. In some cases, the ErbB family inhibitor is a HER3 inhibitor, e.g., an anti-HER3 antibody, such as HMBD-001 (Hummingbird Bioscience). In some cases, the ErbB family inhibitor is a combination of an anti-EGFR antibody and anti-HER2 antibody. In some cases, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino)but-2-enamide). In some cases, the irreversible ErbB family inhibitor is afatinib. In some cases, the irreversible ErbB family inhibitor is dacomitinib. In some cases, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-((3-chloro-4-(3-(trifluoromethyl) phenoxy)phenyl)amino)-5H-pyrrolo[3,2-d]pyrimidin-5-yl)ethyl)-3-hydroxy-3-methylbutanamide), AEE788 ((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl) methoxy]phenyl]-6-[2-[(2-methylsulfonylethylamino)methyl]-1,3-thiazol-4-yl] quinazolin-4-amine). In some cases, the reversible ErbB family inhibitor is sapitinib. In one embodiment, the reversible ErbB family inhibitor is tarloxotinib.

ERK Inhibitors. In some cases, the compounds of the disclosure can be administered simultaneously, separately, or sequentially with an effective amount of an ERK inhibitor in any of the methods described herein. Exemplary ERK inhibitors for use in the methods provided herein include, but are not limited to, ulixertinib, ravoxertinib, CC-90003 (N-[2-[[2-[(2-methoxy-5-methylpyridin-4-ylamino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl) thieno[2.3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187.

FAK Inhibitors. In some cases, the compounds of the disclosure can be administered simultaneously, separately, or sequentially with an effective amount of a FAK inhibitor in any of the methods described herein. Exemplary FAK inhibitors for use in the methods provided herein include, but are not limited to, GSK2256098 (2-[[5-chloro-2-[(S-methyl-2-propan-2-ylpyrazol-3-yl)amino]pyridin-4-yl]amino]-N-methoxybenzamide), PF-00562271 (N-methyl-N-[3-[[12-[(2-oxo-1,3-dihydroindol-5-yl)amino]-5-(trifluoromethyl)pyrimidin-4-yl]amino methyl]pyridin-2-yl|methanesulfonamide). VS-4718 (2-[[2-(2-methoxy-4-morpholin-4-ylanilino)-5-(trifluoromethyl)pyridin-4-yl]amino]-N-methylbenzamide), and APG-2449.

FGFR Inhibitors. In some cases, the compounds of the disclosure can be administered simultaneously, separately, or sequentially with an effective amount of an FGFR inhibitor in any of the methods described herein. Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), debio 1347 ([5-amino-1-(2-methyl-3H-benzimidazol-5-yl) pyrazol-4-yl]-(1H-indol-2-yl) methanone), INCB062079, H3B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

Glutaminase Inhibitors. In some cases, the compounds of the disclosure can be administered simultaneously, separately, or sequentially with an effective amount of a glutaminase inhibitor in any of the methods described herein. Exemplary glutaminase inhibitors for use in the methods provided herein include, but are not limited to, telaglenastat, IPN60090, and OP 330.

IGF-1R Inhibitors. In some cases, the compounds of the disclosure can be administered simultaneously, separately, or sequentially with an effective amount of an IGF-1R inhibitor in any of the methods described herein. Exemplary IGF-1R inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl) piperazin-1-yl]methyl]-2-[(E)-2-(1H-indazol-3-yl) ethenyl]phenyl]-3-methylthiophene-2-carboxamide), PL225B, AVE1642, and BIIB022.

KIF184 Inhibitors. In some cases, the compounds of the disclosure can be administered simultaneously, separately, or sequentially with an effective amount of a KIF18A inhibitor in any of the methods described herein. Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety. In some cases, the KIF18A inhibitor is sovilnesib (AMG 650).

MAT2A inhibitors. In some cases, the compounds of the disclosure can be administered simultaneously, separately, or sequentially with an effective amount of a MAT2A inhibitor in any of the methods described herein. An MAT2A inhibitor is a compound that inhibits methionine adenosyltransferase II alpha. An exemplary MAT2A inhibitor for use in the methods provided herein is AG 270 (3-(cyclohex-1-en-1-yl)-6-(4-methoxyphenyl)-2-phenyl-136zetidindin-2-ylamino)pyrazolo[1,5-a]pyrimidin-7 (4H)-one).

MCL-1 Inhibitors. In some cases, the compounds of the disclosure can be administered simultaneously, separately, or sequentially with an effective amount of a MCL-1 inhibitor in any of the methods described herein. Exemplary MCL-1 inhibitors for use in the methods provided herein include, but are not limited to, murizatoclax, tapotoclax, AZD 5991 ((3aR)-5-chloro-2,11, 12,24,27,29-hexahydro-2,3,24,33-tetramethyl-22H-9,4,8-(metheniminomethyno)-14,20:26,23-dimetheno-10H,20H-pyrazolo[4,3-1][2,15,22,18,19]benzoxadithiadiazacyclohexacosine-32-carboxylic acid). MIK 665 ((αR)-α-[[(5S)-5-[3-Chloro-2-methyl-4-[2-(4-methyl-1-piperazinyl) ethoxy]phenyl]-6-(4-fluorophenyl) thieno[2,3-d]pyrimidin-4-yl]oxy]-2-[[2-(2-methoxyphenyl)-4-pyrimidinyl]methoxy]benzenepropanoic acid), and ABBV-467. In some cases, the MCL-1 inhibitor is murizatoclax. In some cases, the MCL-1 inhibitor is tapotoclax.

MEK Inhibitors. In some cases, the compounds of the disclosure can be administered simultaneously, separately, or sequentially with an effective amount of a MEK inhibitor in any of the methods described herein. Exemplary MEK inhibitors for use in the methods provided herein include, but are not limited to, trametinib, cobimetinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy) imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one). PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554. In some cases, the MEK inhibitor is trametinib.

mTOR Inhibitors. In some cases, the compounds of the disclosure can be administered simultaneously, separately, or sequentially with an effective amount of a mTOR inhibitor in any of the methods described herein. Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib, Torin-1 (1~ (4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4~ (4-(3-methylmorpholino)-7-(oxetan-3-yl)-5,6,7,8-tetrahydropyrido[3,4-d]pyrimidin-2-yl)phenyl)urea), and VS-5584 (SB2343, (S-(8-methyl-2-morpholin-4-yl-9-propan-2-ylpurin-6-yl)pyrimidin-2-amine). In some cases, the mTOR inhibitor is everolimus.

PARP inhibitors. In some cases, the compounds of the disclosure can be administered simultaneously, separately, or sequentially with an effective amount of a PARP inhibitor in any of the methods described herein, A PARP inhibitor is a compound that targets poly(adenosine diphosphate)-ribose polymerase. The term PARP inhibitors encompass PARP1, PARP2, and PARP3 inhibitors. Exemplary PARP inhibitors for use in the methods provided herein include, but are not limited t137zetidinrib, rucaparib, rucaparib camsylate, niraparib, niraparib tosylate, talazoparib, AG-1461, A-966492, PJ34 HCl, niraparib, UPF 1069, ME0328, venadaparib, AZD5305, DR2313, BYK204165, pamiparib, NMS-P118, and NU 1025.

PD-1 Inhibitors. In some cases, the compounds of the disclosure can be administered simultaneously, separately, or sequentially with an effective amount of a PD-1 inhibitor in any of the methods described herein. Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference. In some cases, the PD-1 inhibitor is pembrolizumab. In some cases, the PD-1 inhibitor is the Anti-PD-1 Antibody A.

PD-L1 Inhibitors. In some cases, the compounds of the disclosure can be administered simultaneously, separately, or sequentially with an effective amount of a PD-L1 inhibitor in any of the methods described herein. Exemplary PD-L1 inhibitors for use in the methods provided herein include, but are not limited to, atezolizumab, avelumab, durvalumab, ZKAB001, TG-1501, SHR-1316, MSB2311, MDX-1105, KN035, IMC-001, HLX20, FAZ053, CS1001, CK-301, CBT-502. BGB-A333, BCD-135, and A167. In some cases, the PD-L1 inhibitor is atezolizumab.

PI3K Inhibitors. In some cases, the compounds of the disclosure can be administered simultaneously, separately, or sequentially with an effective amount of a PI3K inhibitor in any of the methods described herein. Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), ME-401 (N-[2-methyl-1-[2~ (1-methylpiperidin-4-yl)phenyl]propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl) ethynyl]-1-oxo-2-phenylisoquinolin-3-yl]ethyl]pyrazolo[1,5-a]pyrimidine-3-carboxamide), SF1126 ((28)-2-[(2S)-3-carboxy-2-[2-[(2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate). XL147 (N-[3-(2,1,3-benzothiadiazol-5-ylamino) quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

PRMT5 Inhibitors. In some cases, the compounds of the disclosure can be administered simultaneously, separately, or sequentially with an effective amount of a PRMT5 inhibitor in any of the methods described herein. A PRMT5 inhibitor in a compound that inhibits protein arginine methyltransferase 5. The term “PRMT5 inhibitor” includes MTA-cooperative PRMT5 inhibitors. Exemplary PRMT5 inhibitors for use in the methods provided herein include, but are not limited to, pemirametostat (6-[(1-acetylpiperidin-4-yl)amino[-N-[(2S)-3-(3,4-dihydro-1H-isoquinolin-2-yl)-2-hydroxypropyl]pyrimidine-4-carboxamide), GSK3203591 (2-(Cyclobutylamino)-N-[(2S)-3-(3,4-dihydro-2 (1H)-isoquinolinyl)-2-hydropropyl]-4-pyridinecarboxamide dihydrochloride)), LLY-283 ((R)-5′-phenyl-7-deazaadenosine; 6-amino-9-[(R)-5′-phenyl(ribofuranosyl)]-7-deazapurine, (2R,3R,4S,5R)-2-(4-Amino-7H-pyrrolo[2,3-d]pyrimidin-7-yl)-5-((R)-hydroxy(phenyl)methyl)tetrahydrofuran-3,4-diol). PRT 811, and MRTX1719 (2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile).

Raf Kinase Inhibitors. In some cases, the compounds of the disclosure can be administered simultaneously, separately, or sequentially with an effective amount of a Raf kinase inhibitor in any of the methods described herein. The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers. The term “Raf kinase inhibitor” as used herein refers to a compound that is capable of negatively modulating or inhibiting all or a portion of the enzymatic activity of one or more member of the Raf family kinases or is capable of disrupting Raf homodimer or heterodimer formation to inhibit activity. In some cases, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5,-morpholino-6′-((tetrahydro-2H-pyran-4-yl)oxy)-[3,3′-bipyridin]-5-yl)-3-(trifluoromethyl)benzamide), LXH254 (N-(3-(2-(2-hydroxyethoxy)-6-morpholinopyridin-4-yl)-4-methylphenyl)-2-(trifluoromethyl) isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-(6,7-dimethoxyquinazolin-4-yl)oxy)phenyl)-3-(5-(1,1,1-trifluoro-2-methylpropan-2-yl) isoxazol-3-yl) urea), CCT196969 (1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide), In some cases, the Raf kinase inhibitor is encorafenib. In some cases, the Raf kinase inhibitor is sorafenib. In some cases, the Raf kinase inhibitor is lifirafenib.

SHP2 Inhibitors. In some cases, the compounds of the disclosure can be administered simultaneously, separately, or sequentially with an effective amount of a SHP2 inhibitor in any of the methods described herein. Exemplary SHP2 inhibitors for use in the methods provided herein include, but are not limited to, SHP-099 (6-(4-amino-4-methylpiperidin-1-yl)-3-(2,3-dichlorophenyl) pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine; vociprotafib (RMC-4630; 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol). In some cases, the SHP inhibitor for use in the methods provided herein is RMC-4630 (vociprotafib, Revolution Medicine). In some cases, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 2172652-48-9). In some cases, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio]pyrazolo[1,5-a]pyrazin-4-yl]-3-methyl-2-oxa-8-azaspiro[4.5]decan-4-amine (CAS 2240982-57-2), 4-[(3S,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (3S,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6). In some cases, the SHP inhibitor for use in the methods provided herein is (1R)-8-[5-(2,3-dichlorophenyl)-6-methylimidazo[1,5-a]pyrazin-8-yl]-8-azaspiro[4.5]decan-4-amine (CAS 2240981-78-4). In some cases, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(1R)-1-amino-8-azaspiro[4.5]dec-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thio]-3-[(3S,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0). 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3). 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol. In some cases, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5). In some cases, the SHP2 inhibitor for use in the methods provided herein is an inhibitor disclosed in U.S. Pat. No. 10,590,090 B2, US 2020/017517 A1, US 2020/017511 A1, WO 2019/075265 A1, or WO 2021/142026, each of which is herewith incorporated by reference in its entirety.

SOS1 Inhibitors. In some cases, the compounds of the disclosure can be administered simultaneously, separately, or sequentially with an effective amount of a SOS1 inhibitor in any of the methods described herein. Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine), BI 1701963, AST-NS2102, MRTX-0902 ((R)-2-methyl-3-(1-((4-methyl-7-morpholinopyrido[3,4-d]pyridazin-1-yl)amino)ethyl)benzonitrile). ERAS-9, RMC-5845, HM-99462, and GH-52.

Src Kinase Inhibitors. In some cases, the compounds of the disclosure can be administered simultaneously, separately, or sequentially with an effective amount of a Src kinase inhibitor in any of the methods described herein. The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily. The term “Src kinase inhibitor” as used herein refers to a compound that is capable of negatively modulating or inhibiting all or a portion of the enzymatic activity of one or more member of the Sre kinases. Exemplary Sre kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl)acetamide). SU6656 ((Z)—N,N-dimethyl-2-oxo-3-((4,5,6,7-tetrahydro-1H-indol-2-yl)methylene)indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl)(2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl)acetamide). In some cases, the Src kinase inhibitor is dasatinib. In some cases, the Src kinase inhibitor is saracatinib. In some cases, the Src kinase inhibitor is ponatinib. In some cases, the Src kinase inhibitor is vandetanib. In some cases, the Src kinase inhibitor is KX-01.

Chemotherapeutic Agents. In some cases, the compounds of the disclosure can be administered simultaneously, separately, or sequentially with an effective amount of one or more chemotherapeutic agents in any of the methods described herein. Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions and General Terminology

The following definitions are provided to assist in understanding the scope of this disclosure. Unless defined otherwise, all technical and scientific terms used herein have the same meaning as is commonly understood by one of ordinary skill in the art to which this disclosure belongs.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence.

This section will define additional terms used to describe the scope of the compounds, compositions and uses disclosed herein.

For purposes of this disclosure, the chemical elements are identified in accordance with the Periodic Table of the Elements, CAS version, Handbook of Chemistry and Physics, 75th Ed. Additionally, general principles of organic chemistry are described in Organic Chemistry, Thomas Sorrell, University Science Books, Sausalito: 1999, and March's Advanced Organic Chemistry, 5th Ed., Ed.: Smith, M. B. and March, J., John Wiley & Sons, New York: 2001, the entire contents of which are hereby incorporated by reference.

As described herein, compounds described herein may optionally be substituted with one or more substituents, such as illustrated generally below, or as exemplified by particular classes, subclasses, and species described herein. It will be appreciated that the phrase “optionally substituted” is used interchangeably with the phrases “substituted or unsubstituted” and “unsubstituted or substituted.” In general, the term “substituted,” whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. In some cases, the each substituent independently is deuterium, halo, oxo, carboxyl, CHO, NH₂, amido, NO₂, ester, thioester, C₀₋₃alkyleneCN, C₁₋₃alkyl, C₁₋₆haloalkyl, C₀₋₆alkylene-OH, C₀₋₃alkylene-C₁₋₄alkoxy, C₀₋₃alkylene-C₁₋₄haloalkoxy, C₀₋₃alkylene-C₁₋₄thioalkoxy, C₀₋₃alkylene-C₁₋₃alkoxy, deuterated C₀₋₆alkylene-OC₁₋₃alkoxy, amido, C₀₋₂alkylene-cycloalkyl having 3-7 total ring atoms, C₀₋₂alkylene-cycloalkenyl having 5-7 total ring atoms, C₀₋₂alkylene-heterocycloalkyl having 3-7 total ring atoms and 1-3 heteroatoms selected from N, O, and S, C₀₋₂alkylene-heterocycloalkenyl having 3-7 total ring atoms and 1-3 heteroatoms selected from N, O, and S, and C₀₋₂alkylene-C₆₋₁₀aryl.

The compounds disclosed herein can be “deuterated,” wherein one or more hydrogen atoms bonded to a carbon atom (C—H) have been replaced with deuterium to form a carbon-deuterium bond (C-D). In some cases, the compounds disclosed herein are deuterated with one deuterium atom. In some cases, the compounds disclosed herein are deuterated with two or three deuterium atoms. In some cases, the compounds disclosed herein are deuterated with four or more deuterium atoms. In some cases, each hydrogen atom of a C—H bond in a particular functional group has been replaced with deuterium. In some cases, the entire compound is deuterated.

Selection of substituents and combinations of substituents contemplated herein are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some cases, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “halo” or “halogen” refers to fluoro (—F), chloro (—Cl), bromo (—Br), and/or iodo (—I).

The term “oxo” refers to a substituent oxygen atom connected to another atom by a double bond (e.g., ═O)). In some cases, a single R group can be an oxo group (e.g., wherein R⁷ is halo, C₁₋₃alkyl, or oxo). In some cases, two geminal R groups, together with the atom to which they are attached, can form an oxo group. Accordingly, the phrase “wherein two geminal R groups together with the atom to which they are attached form an oxo group” results in a ═O group attached to a single atom.

The term “carbonyl” refers to a divalent C═O radical, such as

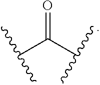 [SMILES: *C(*)=O]

The term “heteroatom,” unless otherwise stated herein, refers to an atom that is not carbon or hydrogen. Examples of heteroatoms include oxygen, sulfur, nitrogen, and phosphorus,

The term “ether” refers to an oxygen atom bonded to two alkyl or aryl groups (R—O—R). The term “ether bridge” refers to an ether group that forms a bridge on a ring, wherein the bridge has the indicated number of carbon atoms. For example, a C₁ ether bridge

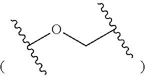 [SMILES: *CO*]

on a cyclohexylene ring cyclohexylene ring can be depicted as, for example,

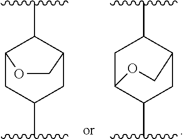 [SMILES: *C1CC2COC(C1)C2* ; *C1CC2OCC1CC2*]

The term “thioether” refers to a sulfur atom bonded to two alkyl or aryl groups (R—S—R). The term “thioether bridge” refers to a thioether group that forms a bridge on a ring, wherein the bridge has the indicated number of carbon atoms. For example, a C₁ thioether bridge

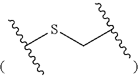 [SMILES: *CS*]

on a cyclohexylene ring cyclohexylene ring can be depicted as, for example,

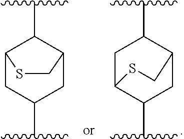 [SMILES: *C1CC2CSC(C1)C2* ; *C1CC2SCC1CC2*]

The term “geminal” refers to two atoms, functional groups, and/or substituents attached to the same atom. Geminal R groups on a chain and ring can be depicted as:

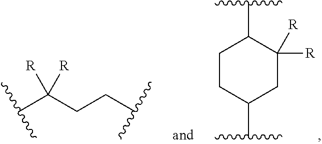

respectively.

The terms “vicinal” and “adjacent” are interchangeable and refer to two atoms, functional groups, and/or substituents that attached to atoms on a chain or a ring that are next to each other.

Vicinal or adjacent R groups on a chain and a ring can be depicted as

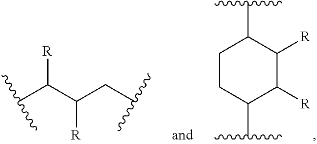

respectively.

The terms “non-neighboring” or “non-adjacent” are interchangeable and refer to two atoms, functional groups, and/or substituents that are attached to atoms on a chain or a ring that are not next to each other (e.g., not adjacent and not geminal). Non-neighboring or non-adjacent R groups on a chain and a ring can be depicted as

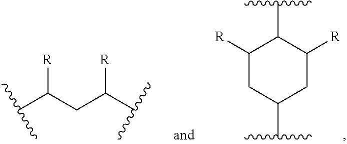

respectively.

The term “alkyl” refers to a saturated straight chain or saturated branched chain hydrocarbon containing the indicated number of carbon atoms. For example, C₃alkyl means the alkyl group has 3 carbon atoms. C₁₋₆alkyl refers to an alkyl group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms). Nonlimiting examples of alkyl groups include methyl, ethyl, n-propyl, isopropyl, n-butyl, sec-butyl, isobutyl, tert-butyl, pentyl, and hexyl.

The term “alkylene” refers to a bivalent saturated aliphatic radical containing the indicated number of carbon atoms. For example, C₃alkylene means the alkylene group has 3 carbon atoms. C₁₋₆alkylene refers to an alkylene group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms). When the number of carbon atoms in an alkylene group is indicated as “C₀,” then the alkylene group is not present and the recited substituent is directly attached to the rest of the compound. For example, the term C₀₋₆alkylene-OH indicates that the OH group can be directly attached to the compound or through a C₁₋₆alkylene linker. Examples of alkylene groups include methylene (—CH₂—), ethylene (—CH₂CH₂—), n-propylene (—CH₂CH₂CH₂—), isopropylene (—CH(CH₃)CH₂—), 1-butylene (—CH₂CH₂CH₂CH₂—), 1-methylbutylene (—CH(CH₃)CH₂CH₂—), 2-methylbutylene (—CH₂CH(CH₃)CH₂—), and 3-methylbutylene (—CH₂CH₂CH₂(CH₃)—).

The term “alkylene bridge” refers to an alkylene group that forms a bridge on a ring, wherein the bridge has the indicated number of carbon atoms. For example, a C₁alkylene bridge

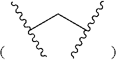 [SMILES: *C*]

on a cyclohexylene ring can be depicted as, for example,

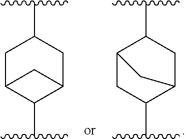 [SMILES: *C1CC2CC(C1)C2* ; *C1CC2CC1CC2*]

A C₂alkylene bridge

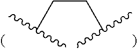 [SMILES: *CC*]

on a cyclohexylene ring can be depicted as, for example,

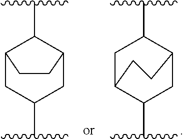 [SMILES: *C1CC2CCC(C1)C2* ; *C1CC2CCC1CC2*]

A C₃alkylene bridge

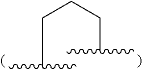 [SMILES: *CCC*]

on a cyclohexylene ring can be depicted as, for example,

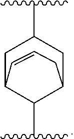 [SMILES: *C1CC2C=CCC(C1)C2*]

The term “haloalkyl” refers to an alkyl group, as previously defined herein, in which one or more of the hydrogen atoms is replaced by a halogen. The halogen is independently selected at each occurrence. The term includes, for example, monohaloalkyl (e.g., CH₂F, CH(CH₂F)CH₃) dihaloalkyl (e.g., CHF₂, CH(CHF₂)CH₃), trihaloalkyl (e.g., CF₃, CH(CF₃)CH₃), and polyhaloalkyl (e.g., CF(CF₃)CH₃). The term further includes perfluorinated alkyl groups, such as CF₃ and CF₂CF₃. For example, the term “C₁₋₄haloalkyl” refers to a C₁₋₄alkyl as defined herein, wherein one or more hydrogen atoms are substituted with a halogen. Representative examples of C₁₋₄haloalkyl include, but are not limited to, CH₂F, CHF₂, CF₃, CHFCl, CH₂CF₃, CFHCF₃, CF₂CF₃, CH(CF₃)₂, CF(CHF₂)₂, CH(CH₂F)(CF₃), CH₂Cl, CHC₂, CCl₃, CHFCl, CH₂CCl₃, CClHCCl₃, CCl₂CCl₃, CH(CCl₃)₂, CCl(CHCl₂), CH(CH₂Cl)CCl₃, and CH₂CF(CH₃)₂.

The term “alkenyl” refers to a straight or branched chain hydrocarbon containing the indicated number of carbon atoms and one or more double bonds. For example, C₃alkenyl means the alkenyl group has 3 carbon atoms. C₂₋₆alkenyl refers to an alkenyl group having a number of carbon atoms encompassing the entire range (e.g., 2, 3, 4, 5, or 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of alkenyl groups include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

The term “alkynyl” refers to a straight or branched chain hydrocarbon containing the indicated number of carbon atoms and one or more triple bonds. For example, C₃alkynyl means the alkynyl group has 3 carbon atoms. C₂₋₆alkynyl refers to an alkynyl group having a number of carbon atoms encompassing the entire range (e.g., 2, 3, 4, 5, and 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of alkynyl groups include ethynyl, 1-propynyl, 2-propynyl, butynyl (e.g., 1-butynyl, 2-butynyl, 3-butynyl), 1-pentynyl, 2-pentynyl, 3-pentynyl, 4-pentynyl, 1-hexynyl, 2-hexynyl, 3-hexynyl, 4-hexynyl, and 5-hexynyl.

The term “hydroxy” or “hydroxyl” refers to an alcohol group (—OH).

The term “alkoxy” or “alkoxyl” refers to an alkyl group having the indicated number of carbon atoms, as previously defined herein, attached to the molecule through an oxygen atom (e.g., (—O-alkyl). For example, a C₃alkoxy group means the alkoxy group has 3 carbon atoms (e.g., OCH₂CH₂CH₃). C₁₋₆alkoxy refers to an alkoxy group having a number of carbon atoms encompassing the entire range (e.g., 2, 3, 4, 5, or 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of alkoxy groups include methoxy, ethoxy, propoxy, iso-propoxy, and butoxy.

The terms “thioalkyl” and “thioalkoxy” are interchangeable and refer to an alkyl group, as previously defined herein, attached to the molecule through a sulfur atom (e.g., —S-alkyl). For example, a C₃thioalkyl group or a C₃thioalkoxy group means the thioalkyl or thioalkoxy group has 3 carbon atoms (e.g., SCH₂CH₂CH₃). C₁₋₆thioalkyl or C₁₋₆thioalkoxy refers to a thioalkyl or thioalkoxy group having a number of carbon atoms encompassing the entire range (e.g., 2, 3, 4, 5, or 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of thioalkyl/thioalkoxy groups include thiomethoxy, thioethoxy, thiopropoxy, thio-iso-propoxy, and butoxy. Additional examples of thioalkyl/thioalkoxy groups include methylthiyl, ethylthiyl, n-propylthiyl, isopropylthiyl, n-butylthiyl, isobutylthiyl, sec-butylthiyl, and tert-butylthiyl.

The terms “haloalkoxy” and “haloalkoxyl” are interchangeable and refer to an alkoxyl group, as previously defined herein, in which one or more of the hydrogen atoms is replaced by a halogen. The halogen is independently selected at each occurrence. The term includes monohaloalkoxy (e.g., OCH₂F, OCH(CH₂F)CH₃) dihaloalkoxy (e.g., OCHF₂, OCH(CHF₂)CH₃), trihaloalkoxy (e.g., OCF₃, OCH(CF₃)CH₃), and polyhaloalkoxy (e.g., OCF(CF₃)CH₃). The term includes perfluorinated alkyl groups, such as OCF₃ and OCF₂CF₃. Representative examples of C₁₋₄haloalkoxy include, but are not limited to, OCH₂F, OCHF₂, OCF₃, OCHFCl, OCH₂CF₃, OCFHCF₃, OCF₂CF₃, OCH(CF₃)₂, OCF(CHF₂), and OCH(CH₂F)(CF₃). Additional examples of C₁₋₄haloalkoxy includes OCH₂Cl, OCHCl₂, OCF₃, OCHFCl, OCH₂CCl₃, OCClHCCl₃, OCCl₂CCl₃, OCH(CCl₃)₂, OCCl(CHCl₂)₂, OCH(CH₂Cl)CCl₃, and OCH₂CF(CH₃)₂.

The term “carbocyclic” or “carbocycle” refers to a ring (saturated, unsaturated, or aromatic) that contains only carbon atoms in the ring. Nonlimiting examples of carbocyclyl rings include cyclopropyl, cyclobutyl, cyclopentyl, 2,3-dihydro-indene, bicyclo[2.2.2]octanyl, adamantyl, spiro[4.4]nonanyl, and naphthalenyl.

The term “cycloalkyl” refers to an aliphatic cyclic hydrocarbon group containing the indicated number of carbon atoms in its ring. For example, C₅cycloalkyl refers to a cycloalkyl group that has 5 carbon atoms in the ring. C₃₋₇cycloalkyl refers to cycloalkyl group having a number of carbon atoms encompassing the entire range (e.g., 3, 4, 5, 6, and 7 carbon atoms in the ring), as well as encompassing all subgroups (e.g., 3-4, 3-5, 3-6, 3-7, 4-5, 4-6, 4-7, 5-6, 5-7, and 6-7 carbon atoms in the ring). The terms “C_{x-y}cycloalkyl” and “a cycloalkyl group having X-Y total ring atoms” are interchangeable. Nonlimiting examples of cycloalkyl groups include cyclopropyl, cyclobutyl, cyclopentyl, and cyclohexyl. The term “spiro-cycloalkyl” refers to a cycloalkyl group as previously defined herein that is attached to the compound through one common atom. For example, a methylpiperidine ring that has a spiro-cyclopropyl group as a substituent can be depicted as:

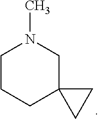

The terms “fused cycloalkyl ring” or “fused-cycloalkyl” can be used interchangeably and refer to a cycloalkyl group, as previously defined herein, that shares two adjacent atoms (i.e., one covalent bond) with the compound to which it is attached. For example, a methylpiperidine ring that has a fused cyclopropyl group as a substituent can be depicted as:

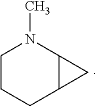

The term “cycloalkenyl” refers to a cyclic hydrocarbon group containing the indicated number of carbon atoms in its ring and one or more double bonds. For example, C₅cycloalkenyl refers to a cycloalkenyl group that has 5 carbon atoms in the ring. C₅₋₇cycloalkenyl refers to cycloalkenyl group having a number of carbon atoms encompassing the entire range (e.g., 5, 6, and 7 carbon atoms in the ring), as well as encompassing all subgroups (e.g., 5-6, 5-7, and 6-7 carbon atoms in the ring). The terms “C_{x-y}cycloalkenyl” and “a cycloalkenyl group having X-Y total ring atoms” are interchangeable. Nonlimiting examples of cycloalkyl groups include cyclopentenyl, and cyclohexenyl. The term “spiro-cycloalkenyl” refers to a cycloalkenyl group as previously defined herein that is attached to the compound through one common atom. For example, a methylpiperidine ring that has a spiro-cyclopentenyl group as a substituent can be depicted as:

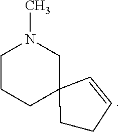

The terms “fused cycloalkenyl ring” or “fused-cycloalkenyl” can be used interchangeably and refers to a cycloalkenyl group, as previously defined herein, that shares two adjacent atoms (i.e., one covalent bond) with the compound to which it is attached. For example, a methylpiperidine ring that has a fused cyclopentenyl group as a substituent can be depicted as:

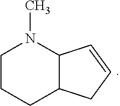

The term “heterocycloalkyl” refers to a saturated ring comprising carbon atoms and 1 or more heteroatoms (e.g., N, O, and/or S), and having the indicated number of total ring atoms (the sum of carbon atoms and heteroatoms in the ring). For example, a heterocycloalkyl group having 5 total atoms and 2 heteroatoms selected from N and S, refers to a ring having 3 carbon atoms and 2 heteroatoms, wherein each heteroatom of the ring independently is N or S. As another example, a heterocycloalkyl group having 5-7 total ring atoms and 1-3 heteroatoms selected from N, O, and S refers to a ring having a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom is independently selected from N, O, and S. Thus, heterocycloalkyl having 5-7 total ring atoms and 1-3 heteroatoms selected from N, O, and S encompasses rings containing, for example, 4 carbon atoms and 1 heteroatom, 3 carbon atoms and 2 heteroatoms, 2 carbon atoms and 3 heteroatoms. 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing is independently selected from N, O, and S. Nonlimiting examples of heterocycloalkyl groups include but are not limited to aziridinyl, azetidinyl, oxetanyl, pyrrolidinyl, pyrazolidinyl, imidazolidinyl, oxazolidinyl, isoxazolidinyl, thiazolidinyl, isothiazolidinyl, tetrahydrofuranyl, tetrahydrothiophenyl, piperidinyl, piperazinyl, tetrahydropyranyl, morpholinyl, azepanyl, diazepanyl, triazepanyl, oxazepanyl, azocanyl, diazocanyl, triazocanyl, oxazocanyl, thiazepanyl, and thiazocanyl. The term “spiro-heterocycloalkyl” refers to a heterocycloalkyl group as previously defined herein that is attached to the compound through one common atom. For example, a methylpiperidine ring that has a spiro-oxetanyl group as a substituent can be depicted as:

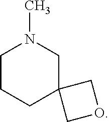

The term “fused-heterocycloalkyl” refers to a heterocycloalkyl group as previously defined herein that shares two adjacent atoms (i.e., one covalent bond) with the compound to which it is attached. For example, a methylpiperidine ring that has a fused-azetidinyl group as a substituent can be depicted as:

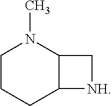

The term “heterocycloalkenyl” is defined similarly to “heterocycloalkyl” except that the ring contains one or more carbon-carbon double bonds. For example, a heterocycloalkenyl group having 5 total atoms and 2 heteroatoms selected from N and S, refers to a ring having at least one double bond, 3 carbon atoms and 2 heteroatoms, wherein each heteroatom of the ring independently is N or S. As another example, a heterocycloalkenyl group having 5-7 total ring atoms and 1-3 heteroatoms selected from N, O, and S refers to a ring having at least one double bond and a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom is independently selected from N, O, and S. Thus, heterocycloalkenyl having 5-7 total ring atoms and 1-3 heteroatoms selected from N, O, and S encompasses rings containing at least one double bone and, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing is independently selected from N, O, and S. Nonlimiting examples of heterocycloalkenyl groups include but are not limited to dihydropyrrolyl, dibydrofuranyl, dihydrothiophenyl, dihydroisoxazolyl, tetrahydropyridyl, dihydropyranyl, and dihydrothiopyranyl. The term “spiro-heterocycloalkeny!” refers to a heterocycloalkenyl group as previously defined herein that is attached to the compound through one common atom. For example, a methylpiperidine ring that has a spiro-dihydropyrrolyl group as a substituent can be depicted as:

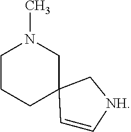

The term “fused-heterocycloalkenyl” refers to a heterocycloalkenyl group as previously defined herein that shares two adjacent atoms (i.e., one covalent bond) with the compound to which it is attached. For example, a methylpiperidine ring that has a fused-dihydrofuranyl group as a substituent can be depicted as:

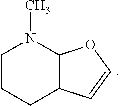

The term “aryl” refers to an aromatic, carbocylic ring having the indicated number of carbon ring atoms. For example, C₆aryl refers to an aryl group that has 6 carbon atoms in the ring (e.g., phenyl), and C₁₀aryl refers to an aryl group that has 10 carbon atoms in the ring (e.g., naphthyl). Aryl groups can be isolated (e.g., phenyl) or fused to another aryl group (e.g., naphthyl or anthracenyl). The term “fused-aryl” refers to an aryl group as previously defined herein that shares two adjacent atoms (i.e., one covalent bond) with the compound to which it is attached. For example, a methylpiperidine ring that has a fused-phenyl group as a substituent can be depicted as:

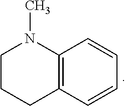

The term “heteroaryl” refers to an aromatic ring comprising carbon and one or more heteroatoms, and having the indicated number of total ring atoms (the sum of carbon atoms and heteroatoms in the ring). For example, a heteroaryl group having 5 total atoms and 2 heteroatoms selected from N and S, refers to an aromatic ring having 3 carbon atoms and 2 heteroatoms, wherein each heteroatom of the ring independently is N or S. As another example, a heteroaryl having 5-7 total ring atoms and 1-3 heteroatoms selected from N, O, and S refers to an aromatic ring having a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom is independently selected from N, O, and S. Thus, heteroaryl having 5-7 total ring atoms and 1-3 heteroatoms selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms. 6 carbon atoms and 1 heteroatom, 5 carbon atoms and 2 heteroatoms, and 4 carbon atoms and 3 heteroatoms, wherein each heteroatom of the foregoing is independently selected from N, O, and S. Nonlimiting examples of heteroaryl groups include but are not limited to furanyl, imidazolyl, isothiazolyl, isoxazolyl, oxadiazolyl, oxazolyl, pyrazolyl, pyrrolyl, thiadiazolyl, thiazolyl, thiophenyl, tetrazolyl, triazinyl, triazolyl, pyridyl, pyridazinyl, pyrazinyl, pyrimidinyl, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quiazolinyl, thiadiazolopyrimidyl, and thienopyridyl. The term “fused-heteroaryl” refers to a heteroaryl group as previously defined herein that shares two adjacent atoms (i.e., one covalent bond) with the compound to which it is attached. For example, a methylpiperidine ring that has a fused-thiazolyl group as a substituent can be depicted as:

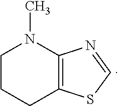

The term “bicyclic ring” refers a functional group that comprises two joined rings. Unless otherwise indicated, the bicyclic ring may be spirocyclic, in which the two rings share a single atom (e.g., a quaternary carbon atom), fused, in which the two rings share two adjacent atoms (i.e, one covalent bond), or bridged, in which to rings share three or more atoms and contain a bridge having at least one atom.

The terms “protecting group” and “protective group” as used herein, are interchangeable and refer to an agent used to temporarily block one or more desired functional groups in a compound with multiple reactive sites. In some cases, a protecting group has one or more, or specifically all, of the following characteristics: (a) is added selectively to a functional group in good yield to give a protected substrate that is (b) stable to reactions occurring at one or more of the other reactive sites; and (c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G, “Protective Groups in Organic Synthesis”, Third Edition, John Wiley & Sons, New York: 1999 (and other editions of the book, such as Wuts, P. G. M. and Greene, T. W. “Greene's Protective Groups in Organic Synthesis,” Fourth Edition, John Wiley & Sons, Hoboken: 2007), wherein the entire contents of each are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene. T. W., Wuts, P. G in “Protective Groups in Organic Synthesis”, Third Edition, John Wiley & Sons, New York: 1999 (and other editions of the book, such as Wuts, P. G. M. and Greene, T. W. “Greene's Protective Groups in Organic Synthesis,” Fourth Edition, John Wiley & Sons, Hoboken: 2007), wherein the entire contents of each are hereby incorporated by reference.

The terms “a bond” and “absent” are used interchangeably to indicate that a specified functional group is not present.

The term “pharmaceutically acceptable” as used herein refers to a composition or a component of a composition that is generally safe, non-toxic, and neither biologically nor otherwise undesirable.

The term “pharmaceutically acceptable salt” as used herein refers to a salt of a compound that is pharmaceutically acceptable and that possesses the desired pharmacological activity of the parent compound. Sach salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use. 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In some cases, the subject is human.

The term “therapeutically effective amount” as used herein refers to that amount of a compound disclosed herein that will elicit the biological or medical response of a tissue, a system, or subject that is being sought by a researcher, veterinarian, medical doctor or other clinician.

The term “metastatic” refers to a cancer that has spread from the place where it first formed to another part of the body. The term non-metastatic refers to a cancer that has not spread from the place where it first formed to another part of the body.

The term “coupled exchange assay” or “2 h coupled exchange assay” as used herein refers to the assay described SECTION 3: Biochemical and Cellular Assays.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula (I) and Formula (II) can be synthesized according to the following schemes. Variables used in the following schemes are the variables as defined for Formula (I) and Formula (II), unless otherwise noted. All starting materials are either commercially available, for example, from Merck Sigma-Aldrich Inc., Fluorochem Ltd., Enamine Ltd, Combi-Blocks, Astech, Oakwood, Thermal Fisher, LabNetwork, Pharm Block, BLD Pharm, AstatTech, Inc., Apollo Scientific, AmBeed, Inc., eNovation Chemicals, and Aurum Pharmatech, or known in the art and may be synthesized by employing known procedures using ordinary skill. Starting materials may also be synthesized via the procedures disclosed herein. Suitable reaction conditions, such as solvent, reaction temperature, and reagents, for the Schemes discussed in this section, may be found in the examples provided herein. The abbreviation PG refers to a protecting group, as defined herein in the DEFINITIONS AND GENERAL TERMINOLOGY section. In the scheme below, each PG can be the same as or different from another PG in the compound, so long as each protecting group can be selectively removed.

In general, the compounds of Formula (I) can be synthesized according to Scheme 1A:

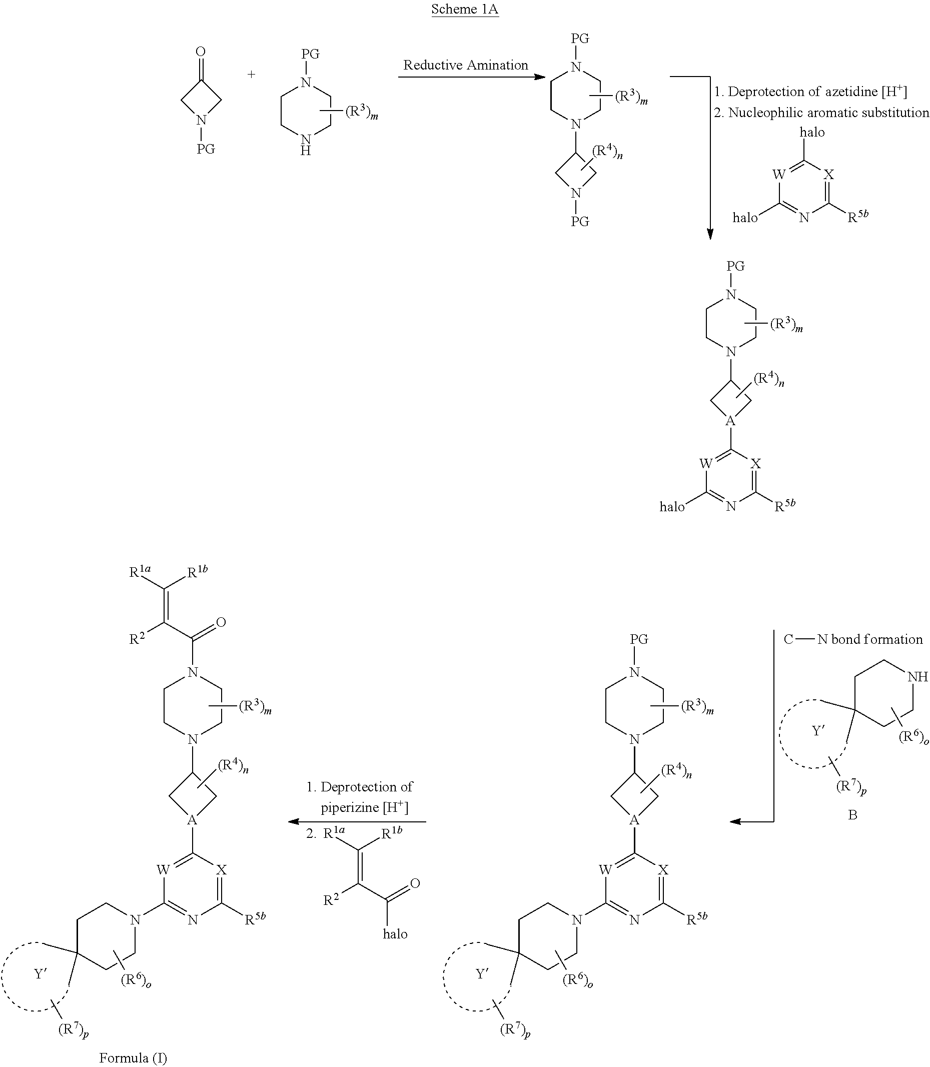

A nitrogen-protected piperazine linker portion of Formula (I):

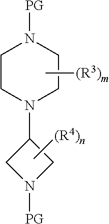

can be synthesized by reacting a desired nitrogen-protected 3-azetidinone with a desired nitrogen-protected piperazine in the presence of an appropriate reducing reagent, such as a borohydride reagent, in a reductive amination reaction. The resulting nitrogen-protected piperazine linker portion of Formula (I) can be coupled to a desired, halogenated pyrimidine or pyridine core:

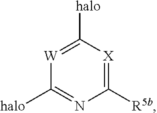

by first deprotecting the nitrogen of the azetidine and then conducting a nucleophilic aromatic substitution reaction in the presence of an appropriate base to form a protected core-linker portion of Formula (I)

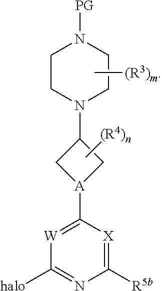

The spiro tail of Formula (I)

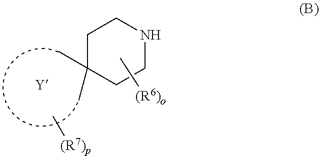

can be synthesized, for example, according to Scheme A or Scheme B.

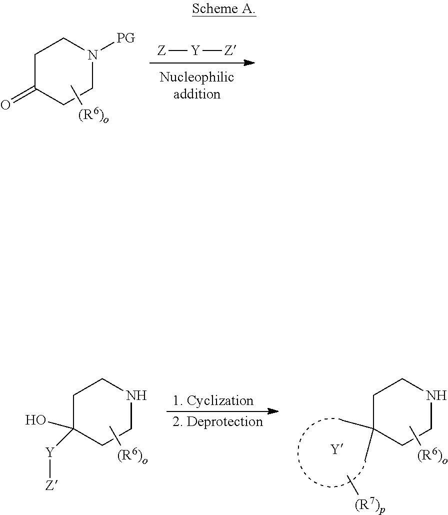

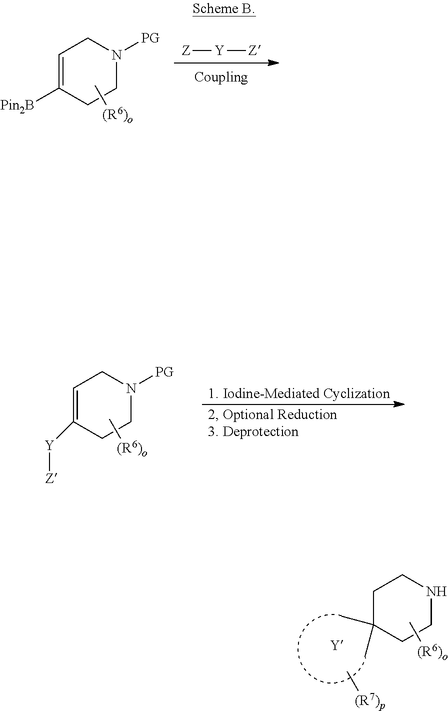

In Scheme A, a desired, optionally substituted, N-protected, piperidin-4-one compound:

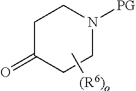

and a desired Z—Y—Z′ compound can undergo a nucleophilic addition reaction to form

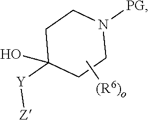

respectively, wherein Z is a functional group capable of nucleophilic addition to the carbonyl on the piperidin-4-one (e.g., an alkyne in an alkynylation reaction, or a halogen in a nucleophilic aromatic substitution reaction), Y is the precursor to the desired Y′ spiro group, and Z′ is a nucleophilic group (e.g., OH) that acts in a subsequent cyclization reaction. In a next step,

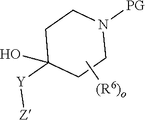

can be subjected to a cyclization and deprotection reaction to form the spiro tail (B):

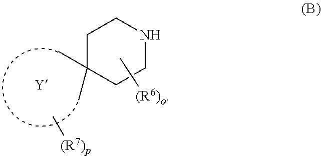

In Scheme B, a boronic ester or acid derived from a substituted, N-protected, piperidin-4-one compound (e.g., a pinacolborane-derived, N-protected, piperidin-4-one compound) and a desired Z—Y—Z′ can undergo a Suzuki coupling reaction, wherein Z is a halogen or leaving group, Y is the precursor to the desired Y′ spiro group, and Z′ is a nucleophilic group (e.g., OH) that acts in a subsequent cyclization reaction to form

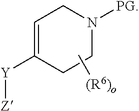

In a next step,

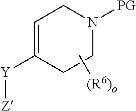

can undergo iodine-mediated cyclization, reduction, and deprotection to form compound (B):

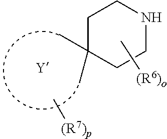

(B). Further reactions can be performed, as necessary, to add desired substituents to the spiro Y′ group. The piperidine tail can be added to the core by, e.g., performing a nucleophilic aromatic substitution reaction in the presence of a base (e.g., DIPEA). Alternatively, the C—N bond can be formed by, e.g., a palladium-catalyzed amination reaction. Finally, the Michael acceptor can be installed on the compound by deprotecting the nitrogen atom of the piperazine ring in the presence of an acid, such as TFA, and reacting the deprotected piperazine ring with a desired halogenated α,β-unsaturated ketone, such as acryloyl chloride to form the compound of Formula (I).

As such, further provided herein is a method for preparing compounds of Formula (I), and pharmaceutically acceptable salts thereof, comprising admixing a compound of Formula (B):

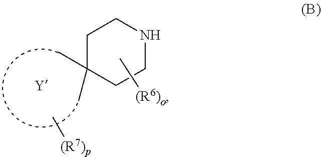

with a protected core-linker portion of Formula (I):

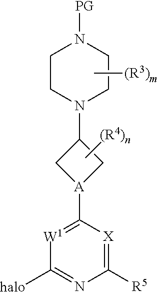

under carbon-nitrogen bond-forming conditions to form intermediates having a structure:

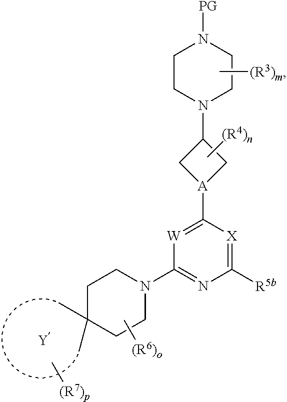

wherein the variables are as previously defined herein. In some cases, the carbon-nitrogen bond forming conditions comprise performing a nucleophilic aromatic substitution reaction in the presence of a base, such as a sterically hindered, nitrogen base (e.g., DIPEA). In some cases, the carbon-nitrogen bond forming conditions comprise a palladium-catalyzed amination reaction. In some cases, the method further comprises deprotecting the piperazine of intermediate:

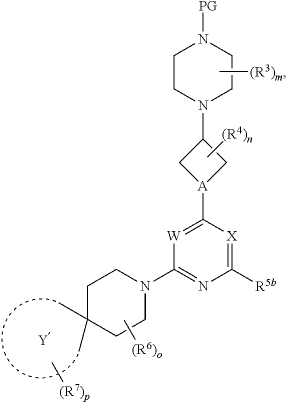

and admixing the resulting compound with

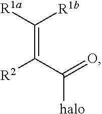

wherein the variables are as previously defined herein, to form Formula (I):

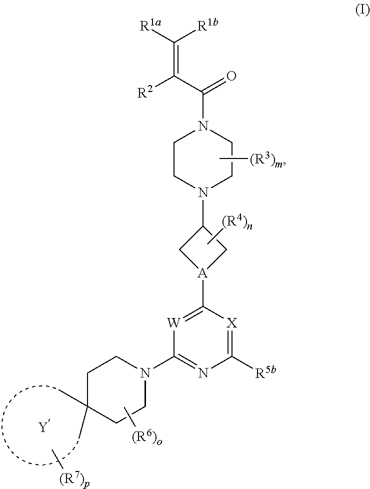

as previously described herein. In some cases, the piperazine is deprotected under acidic conditions. In some cases, the piperazine is deprotected in the presence of a strong acid, such as trifluoroacetic acid (TFA). In some cases, the method further comprises preparing compounds of Formula (B) by cyclizing a compound:

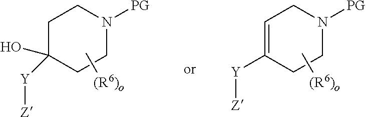

to form

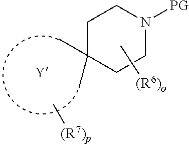

and then deprotecting the cyclized compound to form:

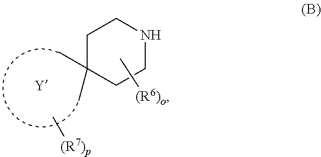

as previously described herein. In some cases, the method further comprises preparing

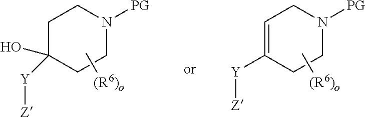

by coupling

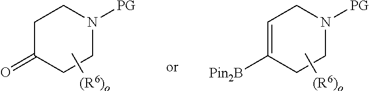

with Z—Y—Z′, as previously described herein, and then subjecting the resulting

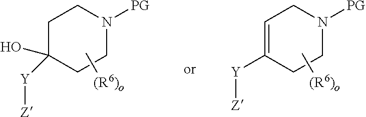

to a cyclization reaction. In some cases, the method further comprises preparing

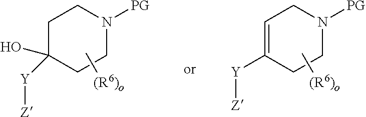

by coupling

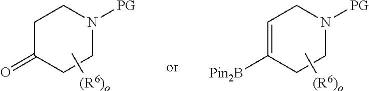

with Z—Y—Z′, as previously described herein, and then subjecting the resulting

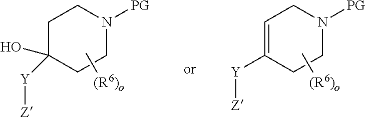

to a cyclization reaction. In some cases, Z is an alkyne and the coupling occurs via an alkynylation reaction. In some cases, Z is a halogen and Y is an aryl or heteroaryl group, and the reaction is a nucleophilic aromatic substitution reaction. In some cases, the method further comprises preparing protected linker portion of Formula (I):

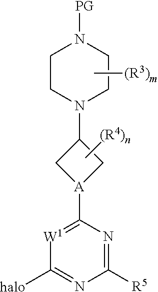

by deprotecting

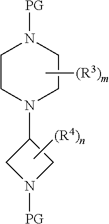

to form

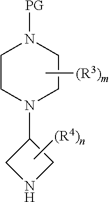

and coupling the deprotected compound to

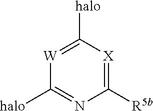

via a nucleophilic aromatic substitution reaction in the presence of an appropriate base. In some cases, the method further comprises preparing a nitrogen-protected linker portion of Formula (I):

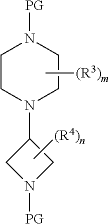

by admixing a desired nitrogen-protected 3-azetidinone with a desired nitrogen-protected piperazine in the presence of an appropriate reducing reagent, such as a borohydride reagent, in a reductive amination reaction.

Compounds of Formula (II) can be prepared similarly to the schemes and methods described above relating to compounds of Formula (I), as shown in Scheme 1B:

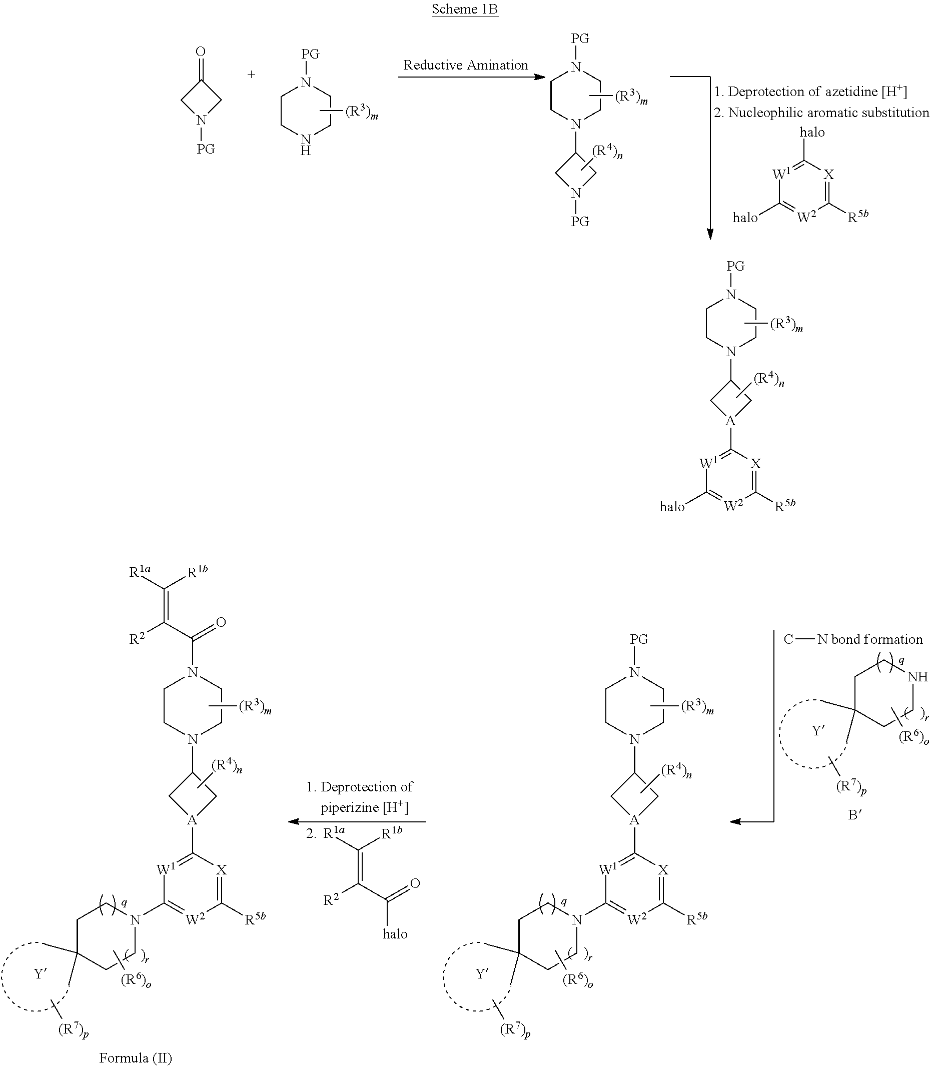

As such, further provided herein is a method for preparing compounds of Formula (II), and pharmaceutically acceptable salts thereof, comprising admixing a compound of Formula (B′):

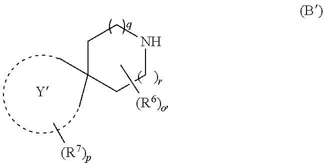

with a protected core-linker portion of Formula (II):

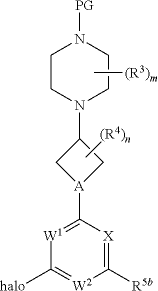

under carbon-nitrogen bond-forming conditions to form intermediates having a structure:

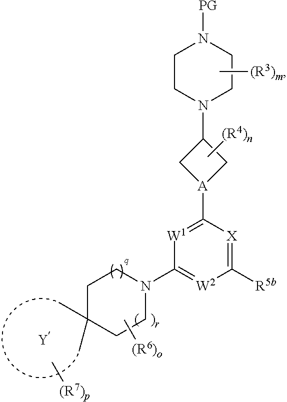

wherein the variables are as previously defined herein. Further provided herein is a method of preparing compounds of Formula (II) wherein q is 1 and r is 1 comprising admixing a compound of Formula (B):

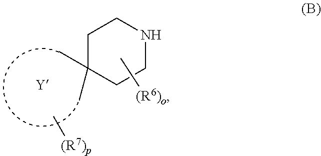

with a protected core-linker portion of Formula (II):

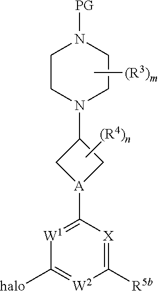

under carbon-nitrogen bond-forming conditions to form intermediates having a structure:

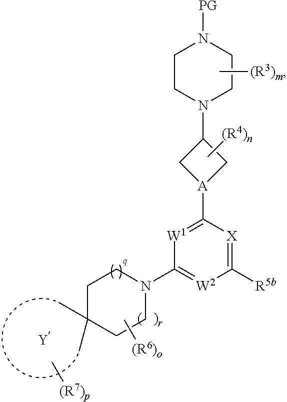

wherein the variables are as previously defined herein. In some cases, the carbon-nitrogen bond forming conditions comprise performing a nucleophilic aromatic substitution reaction in the presence of a base, such as a sterically hindered, nitrogen base (e.g., DIPEA). In some cases, the carbon-nitrogen bond forming conditions comprise a palladium-catalyzed amination reaction. In some cases, the method further comprises deprotecting the piperazine of intermediate:

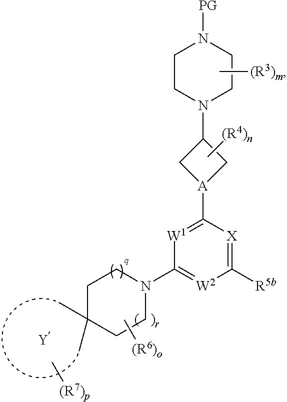

and admixing the resulting compound with

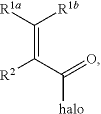

wherein the variables are as previously defined herein, to form Formula (II):

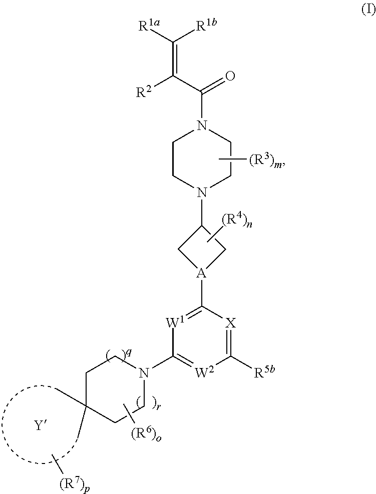

as previously described herein. In some cases, the piperazine is deprotected under acidic conditions. In some cases, the piperazine is deprotected in the presence of a strong acid, such as trifluoroacetic acid (TFA). In some cases, the method further comprises preparing compounds of Formula (B′) using the same methods as described for preparing compounds of Formula (B). In some cases, the method further comprises preparing protected linker portion of Formula (II):

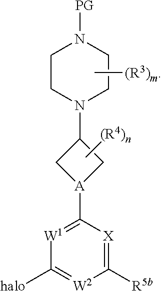

by deprotecting

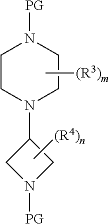

to form

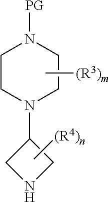

and coupling the deprotected compound to

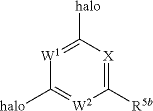

via a nucleophilic aromatic substitution reaction in the presence of an appropriate base. In some cases, the method further comprises preparing a nitrogen-protected linker portion of Formula (II):

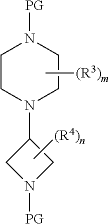

by admixing a desired nitrogen-protected 3-azetidinone with a desired nitrogen-protected piperazine in the presence of an appropriate reducing reagent, such as a borohydride reagent, in a reductive amination reaction.

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid, gas phase, and supercritical fluid), extraction, distillation, trituration, and reverse phase HPLC.

### Intermediates

The disclosure further encompasses intermediate compounds, including structures produced from the synthetic procedures described, whether isolated or generated in-situ and not isolated, prior to obtaining the finally desired compound. These intermediates are included in the scope of this disclosure.

Accordingly, the disclosure further provides a compound of Formula (B):

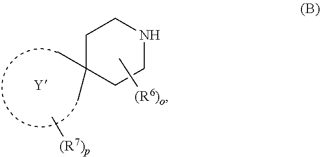

a nitrogen-protected analog, or a pharmaceutically acceptable salt of any of the foregoing, wherein:
is 0, 1, 2, 3, or 4;
p is 0, 1, 2, 3, 4, or 5;
each R⁶ s halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal Re, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₃alkyleneOH, C₀₋₃alkyleneC₁₋₃alkoxy, or C₀₋₃alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₃alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

In some cases, the compound of Formula (B) is a free base (e.g., the nitrogen atom of the piperidine is free:

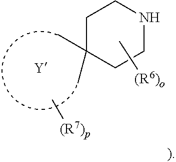

In some cases, the compound of Formula (B) is a pharmaceutically acceptable salt (e.g., a TFA salt). In some cases, the compound of Formula (B) is protected, such as with a nitrogen protecting group (PG):

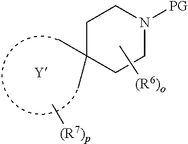

(e.g., di-tert-butyl dicarbonyl or “BOC” group). In some cases, the compound of Formula (B) is selected from the compounds listed in Table E, below, a free base thereof, a nitrogen-protected analog thereof, or a pharmaceutically acceptable salt of any of the foregoing.

TABLE E

| Intermediate No. | Chemical Structure | Name |
|---|---|---|
| B-1 | 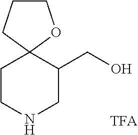 [SMILES: OCC1CNCCC12CCCO2] | 1-oxa-(8-azaspiro[4.5]decan-6- yl)methanol (TFA salt) |
| B-2′ | 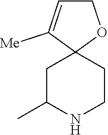 | 4,7-dimethyl-1-oxa-8- azaspiro[4.5]dec-3-ene |
| B-2 | 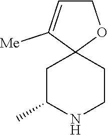 | (7R)-4,7-dimethyl-1-oxa-8- azaspiro[4.5]dec-3-ene |
| B-3′ | 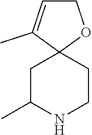 | 4,7-dimethyl-1-oxa-8- azaspiro[4.5]dec-3-ene |
| B-3 | 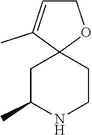 | (7S)-4,7-dimethyl-1-oxa-8- azaspiro[4.5]dec-3-ene |
| B-4 | 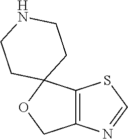 [SMILES: c1nc2c(s1)C1(CCNCC1)OC2] | 4H-spiro[furo[3,4-d]thiazole-6,4′- piperidine] |
| B-5 | 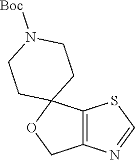 [SMILES: CC(C)(C)OC(=O)N1CCC2(CC1)OCc1ncsc12] | tert-butyl 4H-spiro[furo[3,4- d]thiazole-6,4′-piperidine]-1′- carboxylate |
| B-6 | 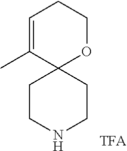 | 5-methyl-1-oxa-9- azaspiro[5.5]undec-4-ene (TFA salt) |
| B-7 | 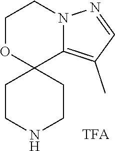 | 3′-methyl-6′,7′- dihydrospiro[piperidine-4,4′- pyrazolo[5,1-c][1,4]oxazine] (TFA salt) |
| B-8 | 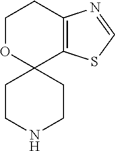 [SMILES: c1nc2c(s1)C1(CCNCC1)OCC2] | 6′,7′-dihydrospiro[piperidine-4,4′- pyrano[4,3-d]thiazole] |
| B-9 | 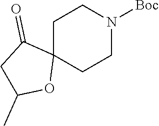 [SMILES: CC1CC(=O)C2(CCN(C(=O)OC(C)(C)C)CC2)O1] | tert-butyl-2-methyl-4-oxo-1-oxa-8- azaspiro[4.5]decane-8-carboxylate |
| B-9-1 | 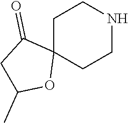 | 2-methyl-1-oxa-8- azaspiro[4.5]decan-4-one |
| B-10 | 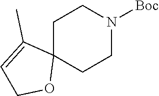 [SMILES: CC1=CCOC12CCN(C(=O)OC(C)(C)C)CC2] | tert-butyl 4-methyl-1-oxa-8- azaspiro[4.5]dec-3-ene-8- carboxylate |
| B-10-1 | 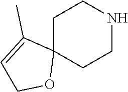 | 4-methyl-1-oxa-8- azaspiro[4.5]dec-3-ene |
| B-11 | 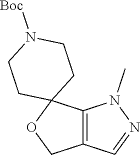 [SMILES: Cn1ncc2c1C1(CCN(C(=O)OC(C)(C)C)CC1)OC2] | tert-butyl 1-methyl-1,4- dihydrospiro[furo[3,4-c]pyrazole- 6,4′-piperidine]-1′-carboxylate |
| B-11-1 | 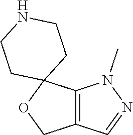 | 1-methyl-1,4- dihydrospiro[furo[3,4-c]pyrazole- 6,4′-piperidine] |
| B-12 | 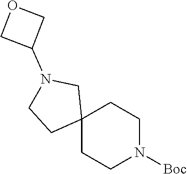 [SMILES: CC(C)(C)OC(=O)N1CCC2(CC1)CCN(C1COC1)C2] | tert-butyl 2-(oxetan-3-yl)-2,8- diazaspiro[4.5]dccane-8- carboxylate |
| B-12-1 | 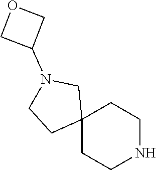 [SMILES: C1CC2(CCN1)CCN(C1COC1)C2] | 2-(oxetan-3-yl)-2,8- diazaspiro[4.5]decane |
| B-13 | 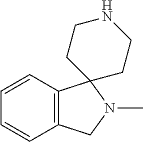 | 2-methylspiro[isoindoline-1.4′- piperidine] |
| B-14 | 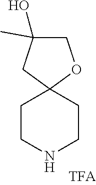 | 3-methyl-1-oxa-8- azaspiro[4.5]decan-3-ol (TFA salt) |
| B-15 | 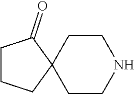 [SMILES: O=C1CCCC12CCNCC2] | 8-azaspiro[4.5]decan-1-one |
| B-16 | 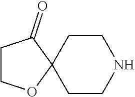 [SMILES: O=C1CCOC12CCNCC2] | 1-oxa-8-azaspiro[4.5]decan-4-one |
| B-17 | 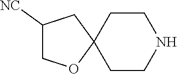 [SMILES: N#CC1COC2(CCNCC2)C1] | 1-oxa-8-azaspiro[4.5]decane-3- carbonitrile |
| B-18′ | 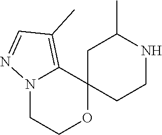 [SMILES: Cc1cnn2c1C1(CCNC(C)C1)OCC2] | 2,3′-dimethyl-6′,7′- dihydrospiro[piperidine-4,4′- pyrazolo[5,1-c][1,4]oxazine] |
| B-18 | 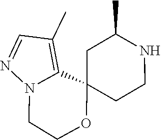 [SMILES: Cc1cnn2c1[C@]1(CCN[C@H](C)C1)OCC2] | (2R,4R)-2,3′-dimethyl-6′,7′- dihydrospiro[piperidine-4,4′- pyrazolo[5,1-c][1,4]oxazine] |
| B-19 | 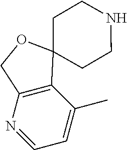 | 4-methyl-7H-spiro[furo[3,4- b]pyridine-5,4′-piperidine] |
| B-20′ | 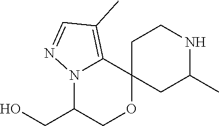 [SMILES: Cc1cnn2c1C1(CCNC(C)C1)OCC2CO] | 2,3′-dimethyl-6′,7′- dihydrospiro[piperidine-4,4′- pyrazolo[5,1-c][1,4]oxazin]-7′- yl)methanol |
| B-20 | 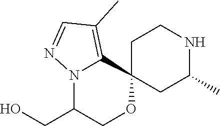 | ((2R,4R)-2,3′-dimethyl-6′,7′- dihydrospiro[piperidine-4,4′- pyrazolo[5,1-c][1,4]oxazin]-7′- yl)methanol |
| B-21′ | 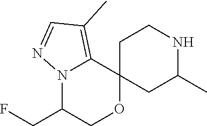 [SMILES: Cc1cnn2c1C1(CCNC(C)C1)OCC2CF] | 7′-(fluoromethyl)-2,3′-dimethyl- 6′,7′-dihydrospiro[piperidine-4,4′- pyrazolo[5,1-c][1,4]oxazine] |
| B-21 | 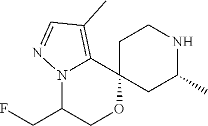 | (2R,4R)-7′-(fluoromethyl)-2,3′- dimethyl-6′,7′- dihydrospiro[piperidine-4,4′- pyrazolo[5,1-c][1,4]oxazine] |
| B-22′ | 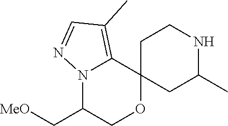 [SMILES: COCC1COC2(CCNC(C)C2)c2c(C)cnn21] | (2R,4R)-7′-(methoxymethyl)-2,3′- dimethyl-6′,7′- dihydrospiro[piperidine-4,4′- pyrazolo[5,1-c][1,4]oxazine] |
| B-22 | 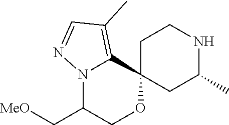 | (2R,4R)-7′-(methoxymethyl)-2,3′- dimethyl-6′,7′- dihydrospiro[piperidine-4,4′- pyrazolo[5,1-c][1,4]oxazine] |
| B-23 | 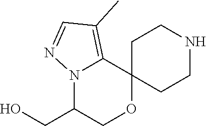 [SMILES: Cc1cnn2c1C1(CCNCC1)OCC2CO] | (3′-methyl-6′,7′- dihydrospiro[piperidine-4,4′- pyrazolo[5,1-c][1,4]oxazin]-7′- yl)methanol |
| B-24 | 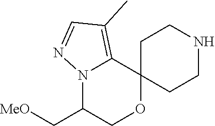 [SMILES: COCC1COC2(CCNCC2)c2c(C)cnn21] | 7′-(Methoxymethyl)-3′-methyl- 6′,7′-dihydrospiro[piperidine-4,4′- pyrazolo[5,1-c][1,4]oxazine] |
| B-25 | 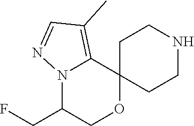 [SMILES: Cc1cnn2c1C1(CCNCC1)OCC2CF] | 7′-(fluoromethyl)-3′-methyl-6′,7′- dihydrospiro[piperidine-4,4′- pyrazolo[5,1-c][1,4]oxazine] |
| B-26 | 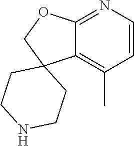 | (4-methyl-2H-spiro[furo[2,3- b]pyridine-3,4′-piperidine] |
| B-27 | 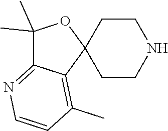 [SMILES: Cc1ccnc2c1C1(CCNCC1)OC2(C)C] | 4,7,7-trimethyl-7H-spiro[furo[3,4- b]pyridine-5,4′-piperidine] |
| B-28 | 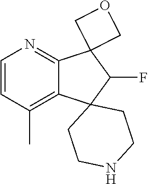 [SMILES: Cc1ccnc2c1C1(CCNCC1)C(F)C21COC1] | 6′-fluoro-4′-methyl-6′H- dispiro[oxetane-3,7′- cyclopenta[b]pyridine-5′,4″- piperidine] |
| B-29 | 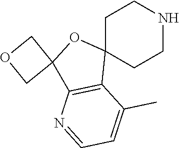 [SMILES: Cc1ccnc2c1C1(CCNCC1)OC21COC1] | 4′-methyldispiro[oxetane-3,7′- furo[3,4-b]pyridine-5′,4″- piperidine] |
| B-30 | 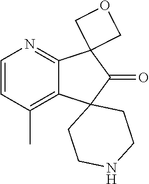 [SMILES: Cc1ccnc2c1C1(CCNCC1)C(=O)C21COC1] | 4′-methyl-6′H-dispiro[oxetane-3,7′- cyclopenta[b]pyridine-5′,4″- piperidin]-6′-one |
| B-31 | 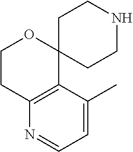 [SMILES: Cc1ccnc2c1C1(CCNCC1)OCC2] | 4′-methyl-7′,8′- dihydrospiro[piperidine-4,5′- pyrano[4,3-b]pyridine] |
| B-32′ | 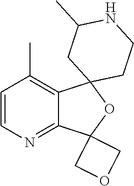 [SMILES: Cc1ccnc2c1C1(CCNC(C)C1)OC21COC1] | 2″,4′-dimethyldispiro[oxetane-3,7′- furo[3,4-b]pyridine-5′,4″- piperidine] |
| B-32 | 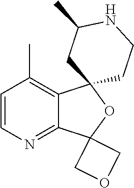 [SMILES: Cc1ccnc2c1[C@]1(CCN[C@H](C)C1)OC21COC1] | (2″R,5′R)-2″,4′- dimethyldispiro[oxetane-3,7′- furo[3.4-b]pyridine-5′,4″- piperidine] |
| B-33 | 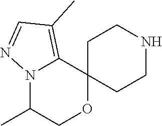 [SMILES: Cc1cnn2c1C1(CCNCC1)OCC2C] | 3′,7′-dimethyl-6′,7′- dihydrospiro[piperidine-4,4′- pyrazolo[5,1-c][1,4]oxazine] |
| B-33-1 | 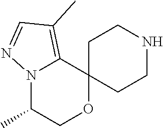 [SMILES: Cc1cnn2c1C1(CCNCC1)OC[C@@H]2C] | (S)-3′,7′-dimethyl-6′,7′- dihydrospiro[piperidine-4,4′- pyrazolo[5,1-c][1,4]oxazine] |
| B-33-2 | 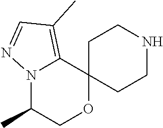 [SMILES: Cc1cnn2c1C1(CCNCC1)OC[C@H]2C] | (S)-3′,7′-dimethyl-6′,7′- dihydrospiro[piperidine-4,4′- pyrazolo[5,1-c][1,4]oxazine] |
| B-34 | 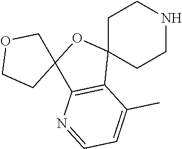 [SMILES: Cc1ccnc2c1C1(CCNCC1)OC21CCOC1] | 4′-methyl-4,5-dihydro-2H- dispiro[furan-3,7′-furo[3,4- b]pyridine-5′,4″-piperidine] |

In some cases, the compound of Formula (B) is selected from Intermediate B-1 to B-34, a free base thereof, a nitrogen-protection version thereof, or a pharmaceutically acceptable salt thereof.

The disclosure further provides piperazine linker intermediates having a structure:

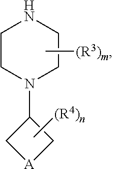

a nitrogen- and/or oxygen-protected analog thereof, or a pharmaceutically acceptable salt of any of the foregoing, wherein the variables are as previously described herein. In some cases, the piperazine linker intermediate is a compound listed in Table F, below, a nitrogen- and/or oxygen-protected analog thereof, or a pharmaceutically acceptable salt of any of the foregoing. In some cases, the nitrogen protecting groups is a BOC or Bn.

TABLE F

| Intermediate | | |
|---|---|---|
| No. | Chemical Structure | Name |
| D1′ | 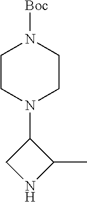 [SMILES: CC1NCC1N1CCN(C(=O)OC(C)(C)C)CC1] | tert-butyl 4-(2-methylazetidin-3- yl)piperazine-1-carboxylate |
| D1 | 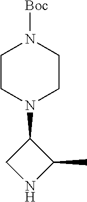 [SMILES: C[C@H]1NC[C@H]1N1CCN(C(=O)OC(C)(C)C)CC1] | tert-butyl 4-((2R,3R)-2- methylazetidin-3-yl)piperazine-1- carboxylate |
| D1-1′ | 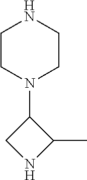 | 4-(2-methylazetidin-3-yl)piperazine |
| D1-1 | 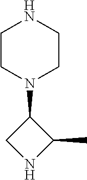 | 4-((2R,3R)-2-methylazetidin-3- yl)piperazine |
| D-2 | 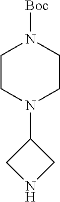 [SMILES: CC(C)(C)OC(=O)N1CCN(C2CNC2)CC1] | tert-butyl 4-(azetidin-3-yl)piperazine- 1-carboxylate |
| D-2-1 | 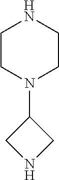 | 1-(azetidin-3-yl)piperazine |
| D-3′ | 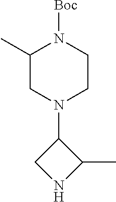 | 1,2-dimethyl-4-(2-methylazetidin-3- yl)piperazine |
| D-3 | 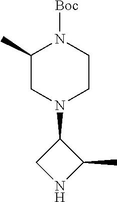 | tert-butyl (R)-2-methyl-4-((2R,3R)-2- methylazetidin-3-yl)piperazine-1- carboxylate |
| D-4′ | 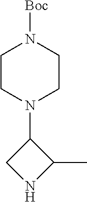 [SMILES: CC1NCC1N1CCN(C(=O)OC(C)(C)C)CC1] | tert-butyl 4-(2-methylazetidin-3- yl)piperazine-1-carboxylate |
| D-4 | 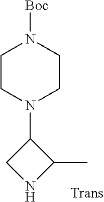 [SMILES: CC1NCC1N1CCN(C(=O)OC(C)(C)C)CC1] | tert-butyl 4-(2-methylazetidin-3- yl)piperazine-1-carboxylate |
| D-4-1 | 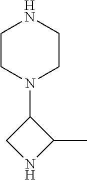 | 1-(2-methylazetidin-3-yl)piperazine |
| D-4-1′ | 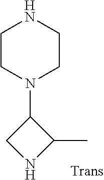 | 1-(2-methylazetidin-3-yl)piperazine |
| D-5′ | 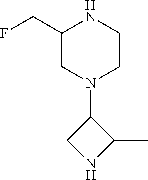 | 3-(fluoromethyl)-1-(2-methylazetidin- 3-yl)piperazine |
| D-5 | 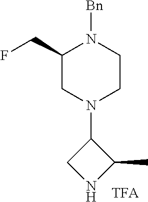 [SMILES: C[C@H]1NCC1N1CCN(Cc2ccccc2)[C@H](CF)C1] | (2S)-1-benzyl-2-(fluoromethyl)-4- ((2R)-2-methylazetidin-3- yl)piperazine trifluoroacetate |
| D-6′ | 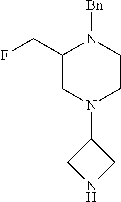 [SMILES: FCC1CN(C2CNC2)CCN1Cc1ccccc1] | (R)-4-(azetidin-3-yl)-1-benzyl-2- (fluoromethyl)piperazine |
| D-6 | 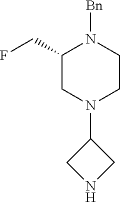 [SMILES: FC[C@H]1CN(C2CNC2)CCN1Cc1ccccc1] | (R)-4-(azetidin-3-yl)-1-benzyl-2- (fluoromethyl)piperazine |
| D-6-1′ | 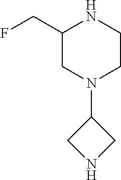 [SMILES: FCC1CN(C2CNC2)CCN1] | 1-(azetidin-3-yl)-3- (fluoromethyl)piperazine |
| D-6-1 | 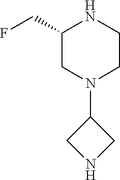 [SMILES: FC[C@H]1CN(C2CNC2)CCN1] | (R)-1-(azetidin-3-yl)-3- (fluoromethyl)piperazine |
| D-7 | 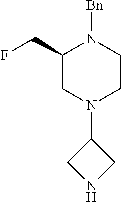 [SMILES: FC[C@@H]1CN(C2CNC2)CCN1Cc1ccccc1] | (S)-4-(azetidin-3-yl)-1-benzyl-2- (fluoromethyl)piperazine |
| D-8′ | 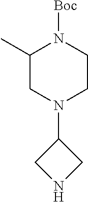 [SMILES: CC1CN(C2CNC2)CCN1C(=O)OC(C)(C)C] | tert-butyl 4-(azetidin-3-yl)-2- methylpiperazine-1-carboxylate |
| D-8 | 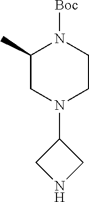 [SMILES: C[C@@H]1CN(C2CNC2)CCN1C(=O)OC(C)(C)C] | tert-butyl (R)-4-(azetidin-3-yl)-2- methylpiperazine-1-carboxylate |
| D-8-1′ | 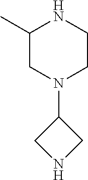 | 1-(azetidin-3-yl)-3-methylpiperazine |
| D-8-1 | 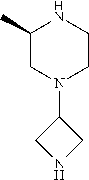 | (R)-1-(azetidin-3-yl)-3- methylpiperazine |
| D-9 | 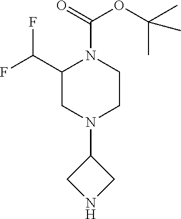 [SMILES: CC(C)(C)OC(=O)N1CCN(C2CNC2)CC1C(F)F] | tert-butyl 4-(azetidin-3-yl)-2- (difluoromethyl)piperazine-1- carboxylate |
| D-9-1 | 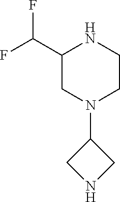 [SMILES: FC(F)C1CN(C2CNC2)CCN1] | 1-(azetidin-3-yl)-3- (difluoromethyl)piperazine |
| D-10 | 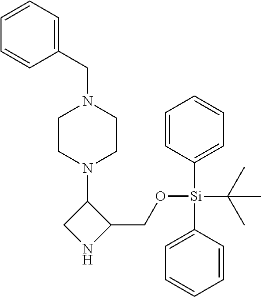 [SMILES: CC(C)(C)[Si](OCC1NCC1N1CCN(Cc2ccccc2)CC1)(c1ccccc1)c1ccccc1] | 1-benzyl-4-(2-(((tert- butyldiphenylsilyl)oxy)methyl) azetidin-3-yl)piperazine |
| D-10-1 | 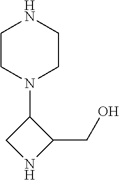 [SMILES: OCC1NCC1N1CCNCC1] | 3-(piperazin-1-yl)azetidin-2- yl)methanol |

In some cases, the piperazine linker intermediate is selected from compound D-1 to D-10-1, a nitrogen- and/or oxygen-protected analog thereof, a nitrogen- and/or oxygen-deprotected analog thereof, or a pharmaceutically acceptable salt of any of the foregoing.

The disclosure further provides intermediates having a structure:

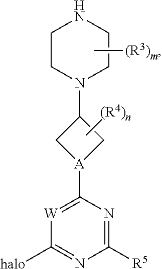

a nitrogen- and/or oxygen-protected analog thereof, or a pharmaceutically acceptable salt of any of the foregoing, wherein the variables are as previously described herein. In some cases, the piperazine linker intermediate is a compound listed in Table G, below, a nitrogen- and/or oxygen-protected analog thereof, or a pharmaceutically acceptable salt of any of the foregoing.

TABLE G

| Intermediate | | |
|---|---|---|
| No. | Chemical Structure | Name |
| E-1′ | 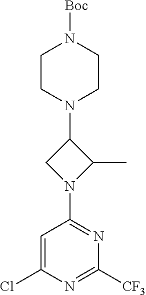 [SMILES: CC1C(N2CCN(C(=O)OC(C)(C)C)CC2)CN1c1cc(Cl)nc(C(F)(F)F)n1] | tert-butyl 4-(1-(6-chloro-2- (trifluoromethyl)pyrimidin-4-yl)-2- methylazetidin-3-yl)piperazine-1- carboxylate |
| E-1 | 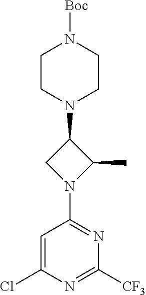 [SMILES: C[C@@H]1[C@H](N2CCN(C(=O)OC(C)(C)C)CC2)CN1c1cc(Cl)nc(C(F)(F)F)n1] | tert-butyl 4-((2R,3R)-1-(6-chloro-2- (trifluoromethyl)pyrimidin-4-yl)-2- methylazetidin-3-yl)piperazine-1- carboxylate |
| E-1-1′ | 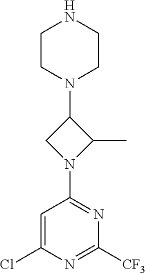 [SMILES: CC1C(N2CCNCC2)CN1c1cc(Cl)nc(C(F)(F)F)n1] | 4-(1-(6-chloro-2- (trifluoromethyl)pyrimidin-4-yl)-2- methylazetidin-3-yl)piperazine |
| E-1-1 | 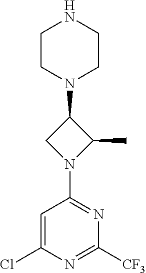 [SMILES: C[C@@H]1[C@H](N2CCNCC2)CN1c1cc(Cl)nc(C(F)(F)F)n1] | 4-((2R,3R)-1-(6-chloro-2- (trifluoromethyl)pyrimidin-4-yl)-2- methylazetidin-3-yl)piperazine |
| E-2′ | 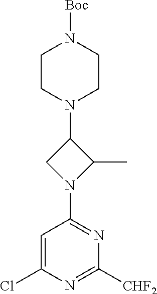 [SMILES: CC1C(N2CCN(C(=O)OC(C)(C)C)CC2)CN1c1cc(Cl)nc(C(F)F)n1] | tert-butyl 4-(1-(6-chloro-2- (difluoromethyl)pyrimidin-4-yl)-2- methylazetidin-3-yl)piperazine-1- carboxylate |
| E-2 | 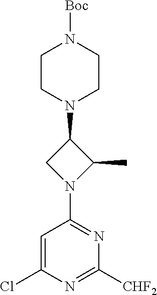 [SMILES: C[C@@H]1[C@H](N2CCN(C(=O)OC(C)(C)C)CC2)CN1c1cc(Cl)nc(C(F)F)n1] | tert-butyl 4-((2R,3R)-1-(6-chloro-2- (difluoromethyl)pyrimidin-4-yl)-2- methylazetidin-3-yl)piperazine-1- carboxylate |
| E-2-2′ | 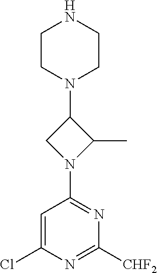 [SMILES: CC1C(N2CCNCC2)CN1c1cc(Cl)nc(C(F)F)n1] | 4-(1-(6-chloro-2- (difluoromethyl)pyrimidin-4-yl)-2- methylazetidin-3-yl)piperazine |
| E-2-2 | 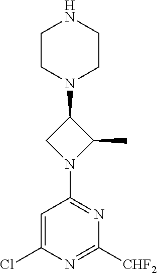 [SMILES: C[C@@H]1[C@H](N2CCNCC2)CN1c1cc(Cl)nc(C(F)F)n1] | 4-((2R,3R)-1-(6-chloro-2- (difluoromethyl)pyrimidin-4-yl)-2- methylazetidin-3-yl)piperazine |
| E-3 | 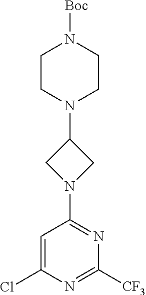 [SMILES: CC(C)(C)OC(=O)N1CCN(C2CN(c3cc(Cl)nc(C(F)(F)F)n3)C2)CC1] | tert-butyl 4-(1-(6-chloro-2- (trifluoromethyl)pyrimidin-4- yl)azetidin-3-yl)piperazine-1- carboxylate |
| E-3′ | 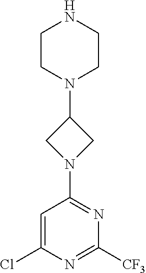 [SMILES: FC(F)(F)c1nc(Cl)cc(N2CC(N3CCNCC3)C2)n1] | 4-(1-(6-chloro-2- (trifluoromethyl)pyrimidin-4- yl)azetidin-3-yl)piperazine |
| E-4 | 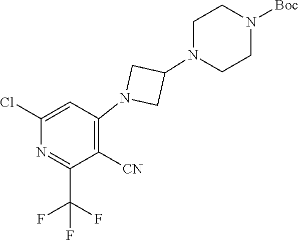 [SMILES: CC(C)(C)OC(=O)N1CCN(C2CN(c3cc(Cl)nc(C(F)(F)F)c3C#N)C2)CC1] | tert-butyl 4-(1-(6-chloro-3-cyano-2- (trifluoromethyl)pyridin-4-yl)azetidin- 3-yl)piperazine-1-carboxylate |
| E-4-1 | 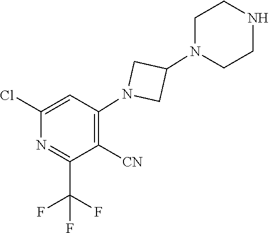 [SMILES: N#Cc1c(N2CC(N3CCNCC3)C2)cc(Cl)nc1C(F)(F)F] | 4-(1-(6-chloro-3-cyano-2- (trifluoromethyl)pyridin-4-yl)azetidin- 3-yl)piperazine |
| E-5′ | 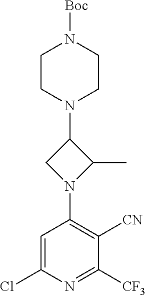 [SMILES: CC1C(N2CCN(C(=O)OC(C)(C)C)CC2)CN1c1cc(Cl)nc(C(F)(F)F)c1C#N] | tert-butyl 4-(1-(6-chloro-3-cyano-2- (trifluoromethyl)pyridin-4-yl)-2- methylazetidin-3-yl)piperazine-1- carboxylate |
| E-5 | 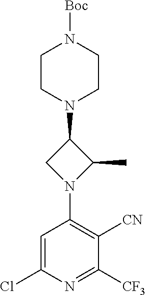 [SMILES: C[C@@H]1[C@H](N2CCN(C(=O)OC(C)(C)C)CC2)CN1c1cc(Cl)nc(C(F)(F)F)c1C#N] | tert-butyl 4-((2R,3R)-1-(6-chloro-3- cyano-2-(trifluoromethyl)pyridin-4- yl)-2-methylazetidin-3-yl)piperazine- 1-carboxylate |
| E-5-1′ | 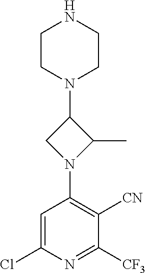 [SMILES: CC1C(N2CCNCC2)CN1c1cc(Cl)nc(C(F)(F)F)c1C#N] | 4-(1-(6-chloro-3-cyano-2- (trifluoromethyl)pyridin-4-yl)-2- methylazetidin-3-yl)piperazine |
| E-5-1 | 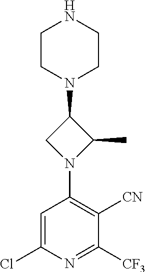 [SMILES: C[C@@H]1[C@H](N2CCNCC2)CN1c1cc(Cl)nc(C(F)(F)F)c1C#N] | 4-((2R,3R)-1-(6-chloro-3-cyano-2- (trifluoromethyl)pyridin-4-yl)-2- methylazetidin-3-yl)piperazine |
| E-6 | 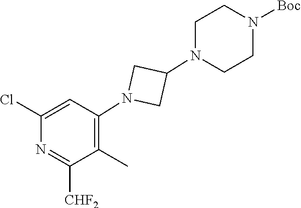 [SMILES: Cc1c(N2CC(N3CCN(C(=O)OC(C)(C)C)CC3)C2)cc(Cl)nc1C(F)F] | tert-butyl 4-(1-(6-chloro-2- (difluoromethyl)-3-methylpyridin-4- yl)azetidin-3-yl)piperazine-1- carboxylate |
| E-6-1 | 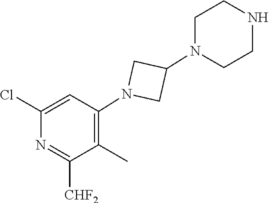 [SMILES: Cc1c(N2CC(N3CCNCC3)C2)cc(Cl)nc1C(F)F] | 4-(1-(6-chloro-2-(difluoromethyl)-3- methylpyridin-4-yl)azetidin-3- yl)piperazine |
| E-7′ | 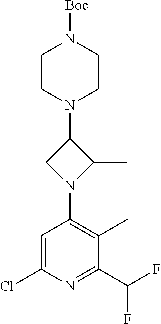 [SMILES: Cc1c(N2CC(N3CCN(C(=O)OC(C)(C)C)CC3)C2C)cc(Cl)nc1C(F)F] | tert-butyl 4-(1-(6-chloro-2- (difluoromethyl)-3-methylpyridin-4- yl)-2-methylazetidin-3-yl) piperazine- 1-carboxylate |
| E-7 | 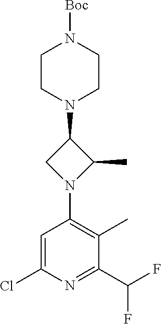 [SMILES: Cc1c(N2C[C@@H](N3CCN(C(=O)OC(C)(C)C)CC3)[C@H]2C)cc(Cl)nc1C(F)F] | tert-butyl 4-((2R,3R)-1-(6-chloro-2- (difluoromethyl)-3-methylpyridin-4- yl)-2-methylazetidin-3-yl) piperazine- 1-carboxylate |
| E-7-1′ | 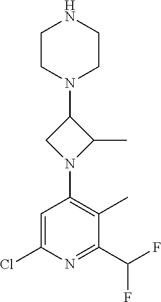 [SMILES: Cc1c(N2CC(N3CCNCC3)C2C)cc(Cl)nc1C(F)F] | 4-(1-(6-chloro-2-(difluoromethyl)-3- methylpyridin-4-yl)-2-methylazetidin- 3-yl) piperazine |
| E-7-1 | 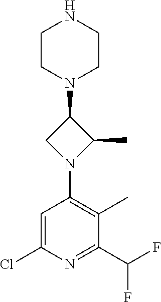 [SMILES: Cc1c(N2C[C@@H](N3CCNCC3)[C@H]2C)cc(Cl)nc1C(F)F] | 4-((2R,3R)-1-(6-chloro-2- (difluoromethyl)-3-methylpyridin-4- yl)-2-methylazetidin-3-yl) piperazine |
| E-8 | 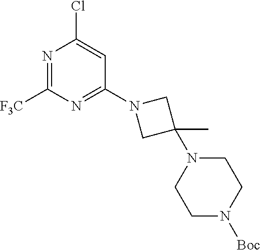 [SMILES: CC(C)(C)OC(=O)N1CCN(C2(C)CN(c3cc(Cl)nc(C(F)(F)F)n3)C2)CC1] | tert-butyl 4-(1-(6-chloro-2- (trifluoromethyl)pyrimidin-4-yl)-3- methylazetidin-3-yl)piperazine-1- carboxylate |
| E-8-1 | 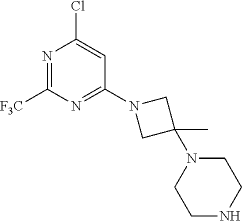 [SMILES: CC1(N2CCNCC2)CN(c2cc(Cl)nc(C(F)(F)F)n2)C1] | 4-(1-(6-chloro-2- (trifluoromethyl)pyrimidin-4-yl)-3- methylazetidin-3-yl)piperazine |

In some cases, the intermediate is selected from compound E-1 to E-8-1, a nitrogen- and/or oxygen-protected analog thereof, a nitrogen- and/or oxygen-deprotected analog thereof, or a pharmaceutically acceptable salt of any of the foregoing.

The disclosure further provides intermediates 3-001.1, 3-001.2, 3-002.1, 3-002.2, 3-003.1, 3-003.2, 3-020.1, 3-020.2, 3-021.1, 3-021.2, 3-046.1, 3.046.2, 3-050.1, 3-050.2, 3-051.1, 3-051.2, 3-051.3, 3-051.4, 3-051.5, 3-052.1, 3-052.2, 3-052.3, 3-052.4, 3-052.5, 3-053.1, 3-053.2, 3-053.3, 3-053.4, 3-053.5, 3-054-1, 3-054-2, 3-055.1, 3-055.2, 3-055.3, 3-055.4, 3-055.5, 3-057.1, 3-057.2, 3-057.3, 3-058.1, 3-058.2-1, 3-058.2.2, 3-058.3, 3-059.1, 3-060.1, 3-060.2, 3-060.3, 3-060.4, 3-060.5, 3-060.6, 3-060.7, 3-060.8, 3-060.9, 3-061.1, 3-061.2, 3-061.3, 3-061.4, 3-061.5, 3-062.1-1, 3-062.1-2, 3-062.1-1, 3-062.2, 3-063.1, 3-063.2, 3-065.1, 3-065.2, 3-065.3, 3-065.4, 3-065.5, 3-067-1, 3-067-2, 3-069.1, 3-069.2, 3-073.1, 3-073.2, 3-073.3, 3-073.4, 3-073.5, 3-074.1, 3-074.2, 3-074.3, 3-074.4, 3-075.1, 3-075.2, 3-075.3, 3-076.1, and 3-076.2, as depicted in SECTION 2: Synthesis of Example Compounds, and pharmaceutically acceptable salts thereof.

Another aspect of the disclosure is a process for preparing a compound or salt described herein (such a compound of Formula (I), Formula (IA), Formula (IB), Formula (II), Formula (IIA), Formula (IIB), Formula (IIC), a compound listed in Table A, a compound listed in Table B, a compound listed in Table C, or a compound listed in Table D, or a pharmaceutically acceptable salt of any of the foregoing), comprising converting an intermediate described herein, such as an intermediate of Formula (B), or an intermediate listed in Table E, F, G, an intermediate selected from the group consisting of 3-001.1, 3-001.2, 3-002.1, 3-002.2, 3-003.1, 3-003.2, 3-020.1, 3-020.2, 3-021.1, 3-021.2, 3-046.1, 3.046.2, 3-050.1, 3-050.2, 3-051.1, 3-051.2, 3-051.3, 3-051.4, 3-051.5, 3-052.1, 3-052.2, 3-052.3, 3-052.4, 3-052.5, 3-053.1, 3-053.2, 3-053.3, 3-053.4, 3-053.5, 3-054-1, 3-054-2, 3-055.1, 3-055.2, 3-055.3, 3-055.4, 3-055.5, 3-057.1, 3-057.2, 3-057.3, 3-058.1, 3-058.2-1, 3-058.2.2, 3-058.3, 3-059.1, 3-060.1, 3-060.2, 3-060.3, 3-060.4, 3-060.5, 3-060.6, 3-060.7, 3-060.8, 3-060.9, 3-061.1, 3-061.2, 3-061.3, 3-061.4, 3-061.5, 3-062.1-1, 3-062.1-2, 3-062.1-1, 3-062.2, 3-063.1, 3-063.2, 3-065.1, 3-065.2, 3-065.3, 3-065.4, 3-065.5, 3-067-1, 3-067-2, 3-069.1, 3-069.2, 3-073.1, 3-073.2, 3-073.3, 3-073.4, 3-073.5, 3-074.1, 3-074.2, 3-074.3, 3-074.4, 3-075.1, 3-075.2, 3-075.3, 3-076.1, and 3-076.2, a protected analog of any of the foregoing, or a pharmaceutically acceptable salt of any of the foregoing into a compound disclosed herein.

### Further Embodiments

Provided herein as Embodiment B1 is a compound of Formula (II):

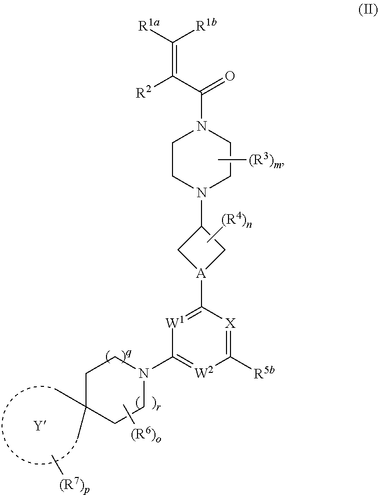

or a pharmaceutically acceptable salt thereof,
wherein:
m is 0, 1, 2, 3, or 4;
n is 0, 1, or 2;
o is 0, 1, 2, 3, or 4;
p is 0, 1, 2, 3, 4, or 5;
q is 0, 1, or 2;
r is 0 or 1;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl. C—C₂₋₃alkynyl, C—C₁₋₃haloalkyl, C—C₀₋₃alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkyleneOH, or C₀₋₃alkyleneC₁₋₄alkoxy;
X is N or C—R^{5a},
Y′ is C₃₋₇cycloalkyl, C₄₋₇cycloalkenyl, heterocycloalkyl having 3 to 7 total ring atoms and 1-3 heteroatoms independently selected from N, O, and S. or heterocycloalkenyl having 5 to 7 total ring atoms and 1-3 heteroatoms independently selected from N, O, and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₃alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form

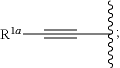

each R³ independently is C₁₋₃alkyl, C₁₋₃haloalkyl,

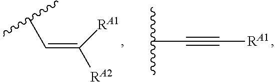

C₀₋₃alkyleneCN, C₀₋₃alkyleneOH, C₀₋₃alkylene-C₁₋₃alkoxy, oxo, spiro-C₃₋₇cycloalkyl, spiro-C₄₋₇cycloalkenyl, spiro-heterocycloalkyl having 3-7 total ring atoms and 1 or 2 heteroatoms independently selected from N, O, and S, or spiro-heterocycloalkenyl having 4-7 total ring atoms and 1 or 2 heteroatoms independently selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms independently selected from N, O, and S, or fused-heterocycloalkenyl having 4-7 total ring atoms and 1 or 2 heteroatoms independently selected from N, O, and S;
each of R^{A1} and R^{A2} independently is H, C₁₋₃alkyl, C₁₋₃haloalkyl, or C₃₋₅cycloalkyl;
each R⁴ independently is C₁₋₃alkyl, C₁₋₃haloalkyl, C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 heteroatoms independently selected from N, O and S;
R^{5a} is H, CN, halo, C₁₋₃alkyl, C₁₋₃haloalkyl, C₂₋₆alkenyl, C₂₋₆alkynyl, C₀₋₃alkylencOH, C₀₋₃alkylene-C₁₋₃alkoxy, or C₃₋₅cycloalkyl;
R^{5b} is halo, C₁₋₃haloalkyl, C₁₋₃alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms independently selected from N, O, and S, heterocycloalkenyl having 5-7 total ring atoms and 1-3 heteroatoms independently selected from N, O, and S, or R^{5a} and R^{5b}, together with the atoms to which they are attached, form C₃₋₇cycloalkyl;
wherein each of the foregoing C₁₋₆alkyl, C₂₋₆alkenyl, C₂₋₄alkynyl, C₃₋₇cycloalkyl, C₅₋₇cycloalkenyl, heterocycloalkyl, and heterocycloalkenyl independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl;
each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₃alkyleneC₁₋₃alkoxy, or C₀₋₃alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

Provided herein as Embodiment B2 is the compound or salt of Embodiment B1, wherein at least one of R^{1a}, R^{1b}, and R² is H or D.

Provided herein as Embodiment B3 is the compound or salt of Embodiment B1 or B2, wherein each of R^{1a}, R^{1b}, and R² independently is H or D.

Provided herein as Embodiment B4 is the compound or salt of Embodiment B1 or B2, wherein at least one of R^{1a}, R^{1b}, and R² is halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₃alkylene-CN, C₀₋₃alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form

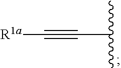

wherein each R^{N1} independently is H or CH₃.

Provided herein as Embodiment B5 is the compound or salt of Embodiment B4, wherein at least one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₂, CH₂N(CH₃)₂, aziridin-1-yl-methyl, azetidin-1-yl-methyl, pyrrolidine-1-yl-methyl, piperidin-1-yl-methyl, or morpholin-1-yl-methyl.

Provided herein as Embodiment B6 is the compound or salt of Embodiment B1 or B2, wherein

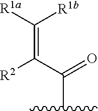

is

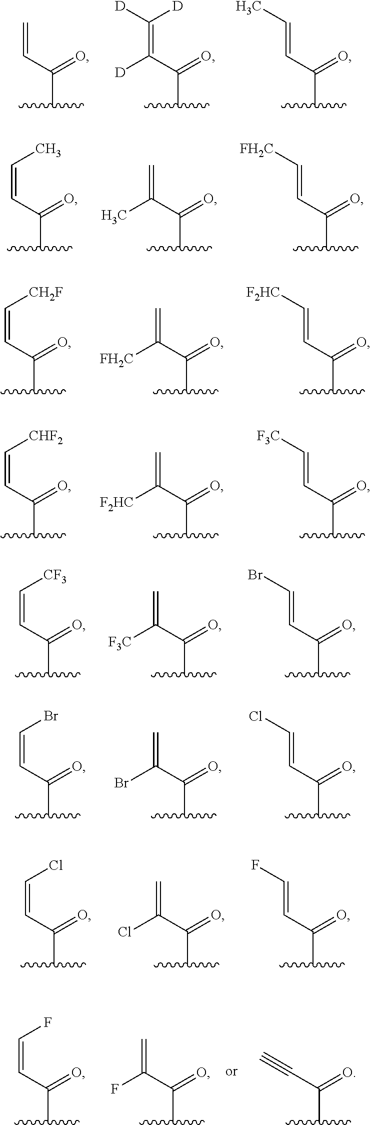

Provided herein as Embodiment B7 is the compound or salt of Embodiment B6, wherein

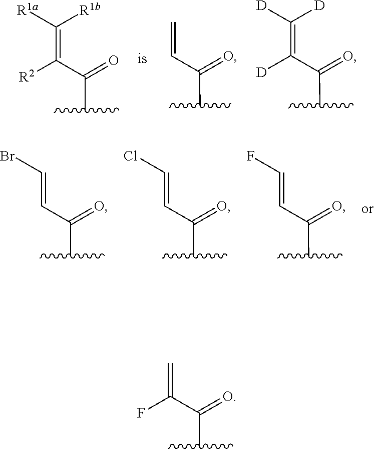

Provided herein as Embodiment B8 is the compound or salt of Embodiment B7, wherein

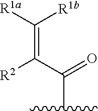

is

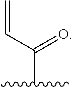 [SMILES: *C(=O)C=C]

Provided herein as Embodiment B9 is the compound or salt of any one of Embodiments B1-8, wherein m is 0 or 1.

Provided herein as Embodiment B10 is the compound or salt of any one of Embodiments B1-8, wherein m is 2, 3, or 4.

Provided herein as Embodiment B11 is the compound or salt of any one of Embodiments B1-10, wherein at least one R³ is C₁₋₃alkyl or C₁₋₃haloalkyl.

Provided herein as Embodiment B12 is the compound or salt of Embodiment B11, wherein at least one R³ is CH₃, CH₂CH₃, CF₃, CHF₂, or CH₂F.

Provided herein as Embodiment B13 is the compound or salt of any one of Embodiments B1-10, wherein at least one R³ is

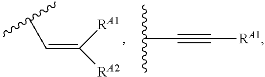

C₀₋₃alkyleneCN, C₁₋₃alkyleneOH, C₀₋₃alkylene-C₁₋₃alkoxy, oxo, spiro-C₃₋₇cycloalkyl, spiro-heterocycloalkyl having 3-7 total ring atoms and 1 or 2 heteroatoms independently selected from N. O and S, spiro-C₄₋₇cycloalkenyl, spiro-heterocycloalkenyl having 4-7 total ring atoms and 1 or 2 heteroatoms independently selected from N, O and S; two vicinal R³, together with the atoms to which they are attached, form fused-C₃₋₇cycloalkyl, fused-heterocycloalkyl having 3-7 total ring atoms and 1 or 2 heteroatoms independently selected from N, O and S, fused-C₄₋₇cycloalkenyl, or fused-heterocycloalkenyl having 4-7 total ring atoms and 1 or 2 heteroatoms independently selected from N, O and S; wherein each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl.

Provided herein as Embodiment B14 is the compound or salt of Embodiment B13, wherein at least one R³ is

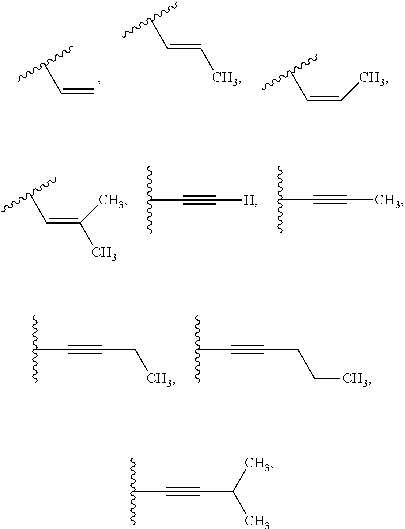

CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, spiro-tetrahydrofuranyl, or two vicinal R³, together with the atoms to which they are attached, form fused-cyclopropyl or fused-cyclobutyl.

Provided herein as Embodiment B15 is the compound or salt of any one of Embodiments B1-10, wherein each R³ independently is CH₃, CH₂CH₃, CF₃, CHF₂, CH₂F,

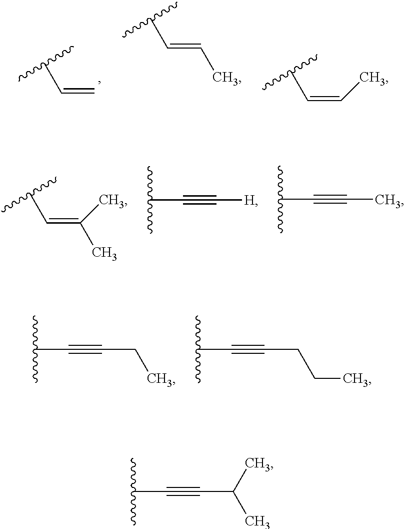

CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl.

Provided herein as Embodiment B16 is the compound or salt of any one of Embodiments B1-8, wherein m is 0; or m is 1 and R³ is CH₃, CH₂F, CHF₂, CF₃. CN, CH₂CN, CH₂OH, CH₂OCH₃, or spiro-oxetanyl.

Provided herein as Embodiment B17 is the compound or salt of Embodiment B16, wherein m is 0; or m is 1 and R³ is CH₃.

Provided herein as Embodiment B18 is the compound or salt of any one of Embodiments B1-8, wherein

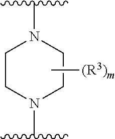

is

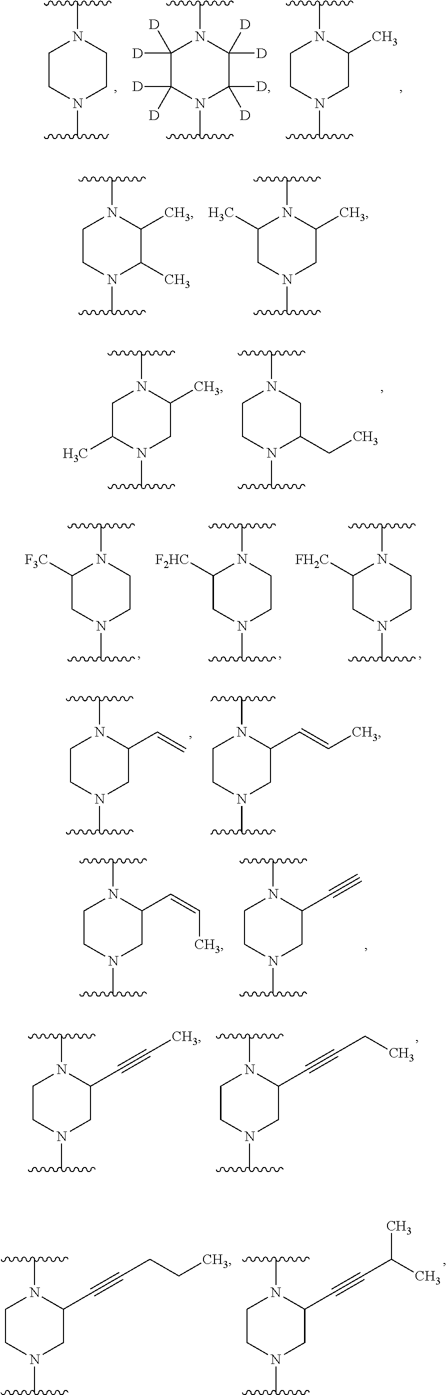

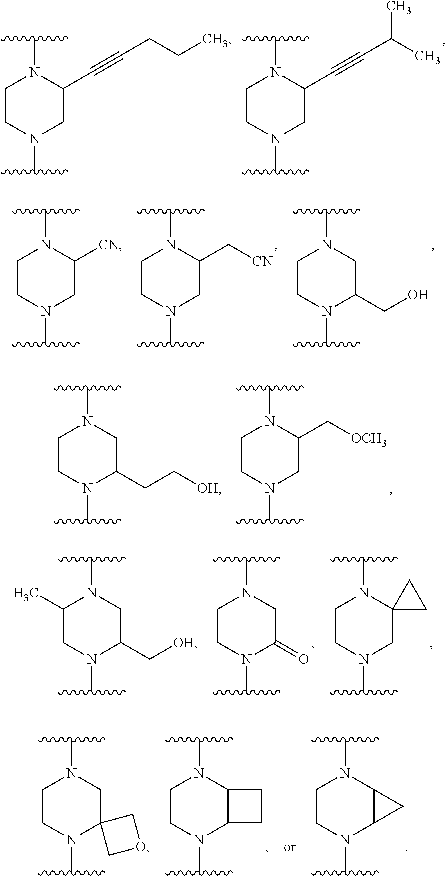

Provided herein as Embodiment B19 is the compound or salt of Embodiment B18, wherein

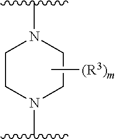

is

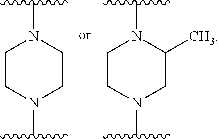

Provided herein as Embodiment B20 is the compound or salt of any one of Embodiments B1-19, wherein A is N.

Provided herein as Embodiment B21 is the compound or salt of any one of Embodiments B1-19, wherein A is CH, C-halo, C—CN, C—C₁₋₃alkyl. C—C₁₋₃haloalkyl, C—C₀₋₃alkyleneOH, or C—C₀₋₃alkylene-C₁₋₄alkoxy.

Provided herein as Embodiment B22 is the compound or salt of Embodiment B21, wherein A is CH, C—F, C—Cl, C—CN, C—CH₃, C—CH₂F, C—CHF₂, C—CF₃, C—OH, C—CH₂OH, C—OCH₃, or C—CH₂OCH₃.

Provided herein as Embodiment B23 is the compound or salt of any one of Embodiments B1-22, wherein n is 0 or 1.

Provided herein as Embodiment B24 is the compound or salt of any one of Embodiments B1-23, wherein at least one R⁴ is C₁₋₃alkyl or C₁₋₃haloalkyl.

Provided herein as Embodiment B25 is the compound or salt of Embodiment B24, wherein at least one R⁴ is CH₃, CH₂CH₃, CH₂CH₂CH₃, CH(CH₃)₂, CH₂F, CHF₂, or CF₃.

Provided herein as Embodiment B26 is the compound or salt of any one of Embodiments B1-23, wherein at least one R⁴ is C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 heteroatoms independently selected from N, O and S.

Provided herein as Embodiment B27 is the compound or salt of Embodiment B26, wherein at least one R⁴ is CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl.

Provided herein as Embodiment B28 is the compound or salt of any one of Embodiments B1-23, wherein each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl.

Provided herein as Embodiment B29 is the compound or salt of Embodiment B28, wherein

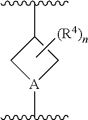

is

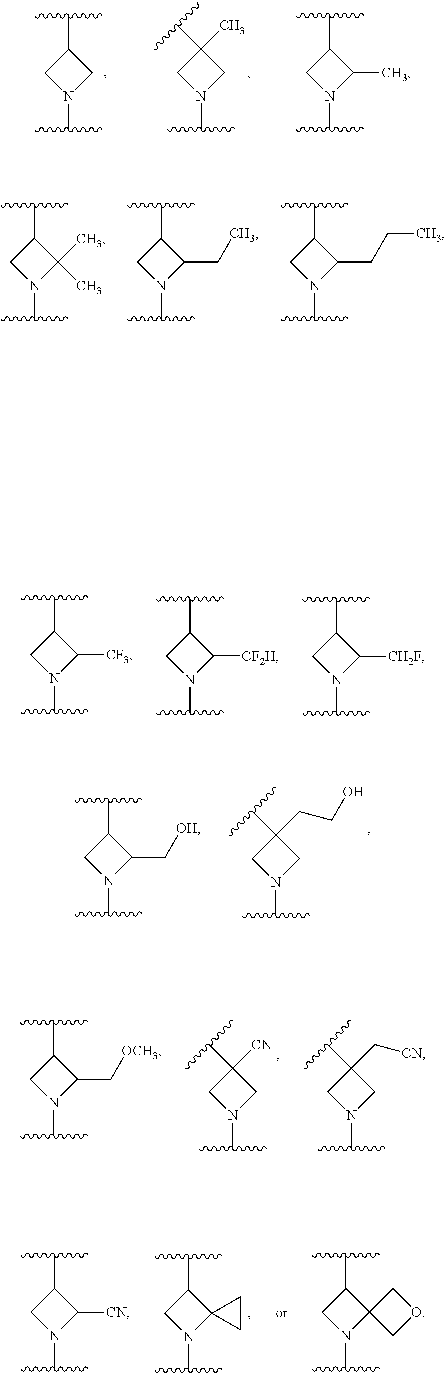

Provided herein as Embodiment B30 is the compound or salt of Embodiment B29, wherein

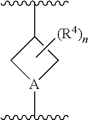

is

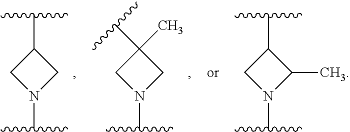

Provided herein as Embodiment B31 is the compound or salt of any one of Embodiments B1-30, wherein W¹ is N, CH, C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), or C—CCH, C—OH, C—CH₂OH, C—OCH₃, or C—CH₂OCH₃.

Provided herein as Embodiment B32 is the compound or salt of Embodiment B31, wherein W¹ is CH.

Provided herein as Embodiment B33 is the compound or salt of any one of Embodiments B1-32, wherein W² is N, CH, C—F, C—Cl, C—CN, C—CH, C—CH—CH₃, C—CH₂F, C—CHF₂, C—CF₃, C—CH═CH₂, C—C(OH)═CH₂, C—CH═CH(OH), or C—CCH, C—OH, C—CH₂OH, C—OCH₃, or C—CH₂OCH₃.

Provided herein as Embodiment B34 is the compound or salt of Embodiment B33, wherein W² is N.

Provided herein as Embodiment B35 is the compound or salt of any one of Embodiments B1-30, wherein W¹ is CH and W² is N.

Provided herein as Embodiment B36 is the compound or salt of any one of Embodiments B1-35, wherein X is N.

Provided herein as Embodiment B37 is the compound or salt of any one of Embodiments B1-35, wherein X is C—R^{5a} and R^{5a} is H, CN, Br, Cl, F, CH₃, CH₂CH₃, CF₃, CHF₂, CH₂F,

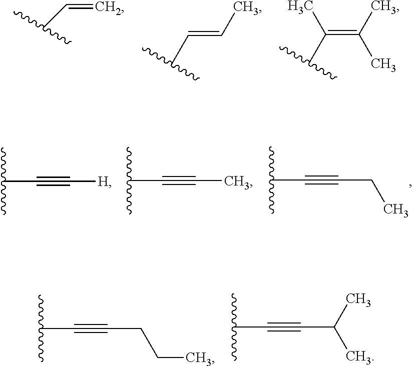

OH, CH₂OH, OCH₃, CH₂OCH₃, or

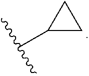 [SMILES: *C1CC1]

Provided herein as Embodiment B38 is the compound or salt of Embodiment B37, wherein R^{5a} is CN or CH₃.

Provided herein as Embodiment B39 is the compound or salt of any one of Embodiments B1-35, wherein X is N, C—CH₃, or C—CN.

Provided herein as Embodiment B40 is the compound or salt of any one of Embodiments B1-30, wherein W¹ is CH; W² is N; and X is N, C—CH₃, or C—CN.

Provided herein as Embodiment B42 is the compound or salt of any one of Embodiments B1-40, wherein R^{5b} is Br, Cl, F, CF₃, CF₂H, CFH₂, CF₂CH₃, OCH₃, SCH₃ CH₃, CH₂CH₃, CH₂CH₂CH₃, CH(CH₃)₂,

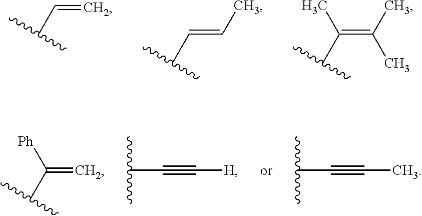

Provided herein as Embodiment B42 is the compound or salt of Embodiment B41, wherein R^{5b} is CF₃ or CF₂H.

Provided herein as Embodiment B43 is the compound or salt of any one of Embodiments B1-30, wherein

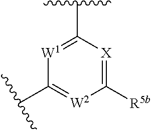

is

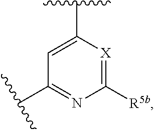

X is N, C—CH₃, or C—CN; and R^{b} is CF₃ or CF₂H.

Provided herein as Embodiment B44 is the compound or salt of any one of Embodiments B1-30, wherein

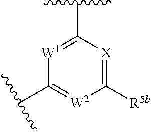

is

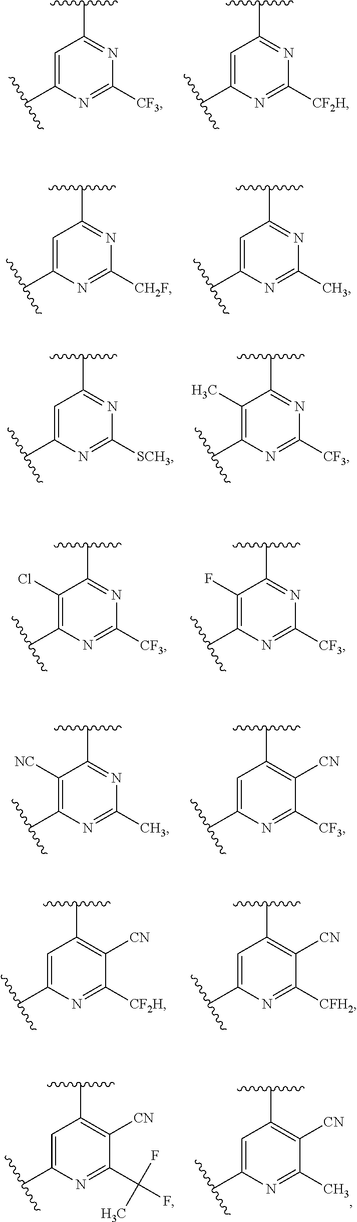

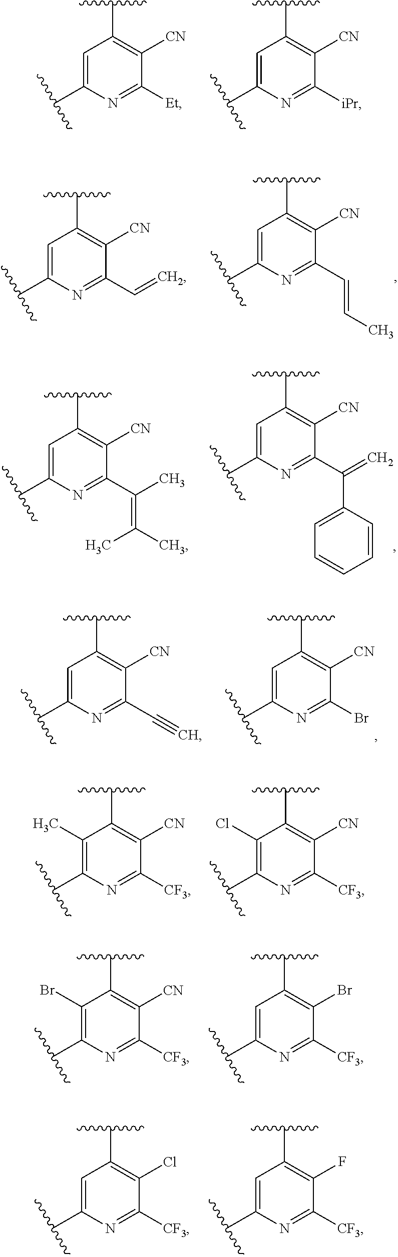

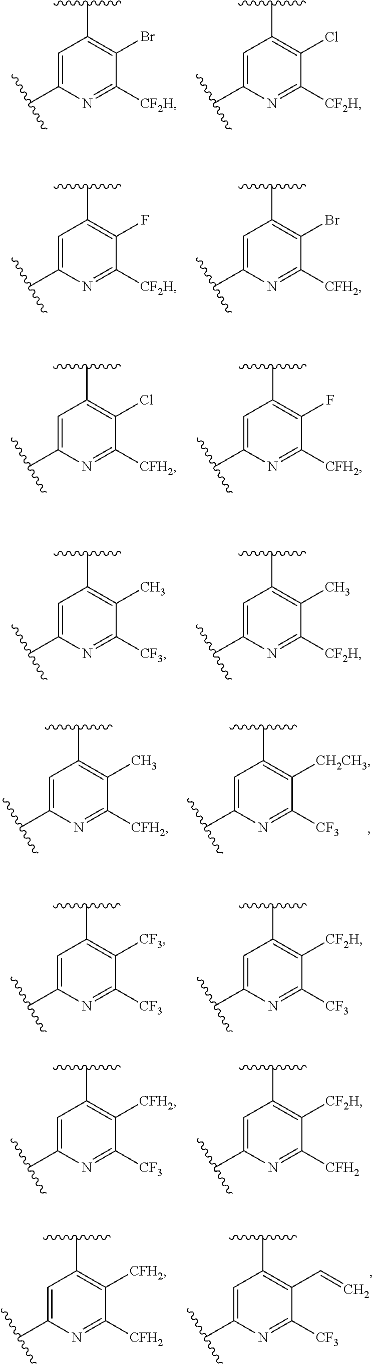

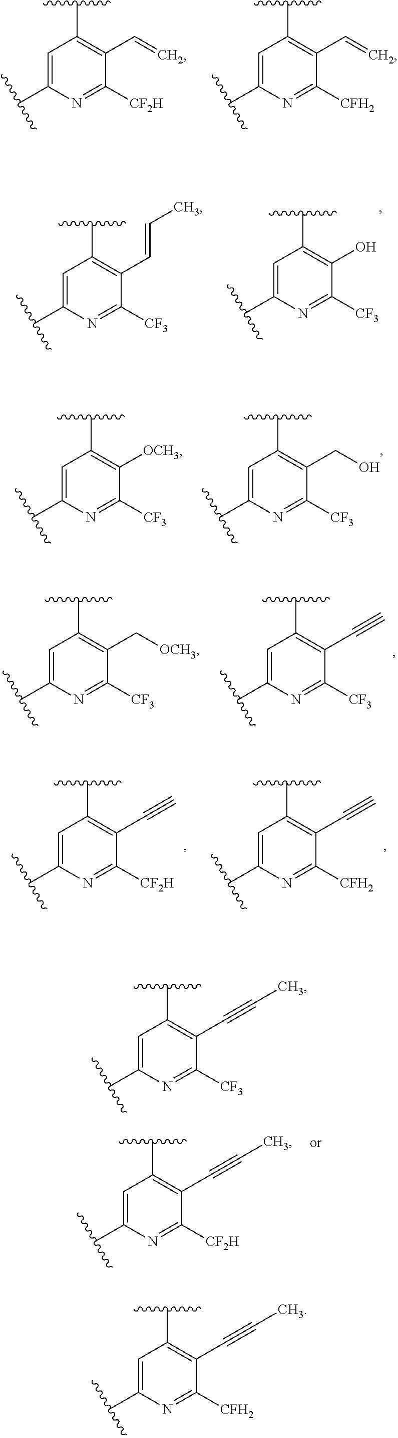

Provided herein as Embodiment B45 is the compound or salt of Embodiment B44, wherein

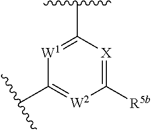

is

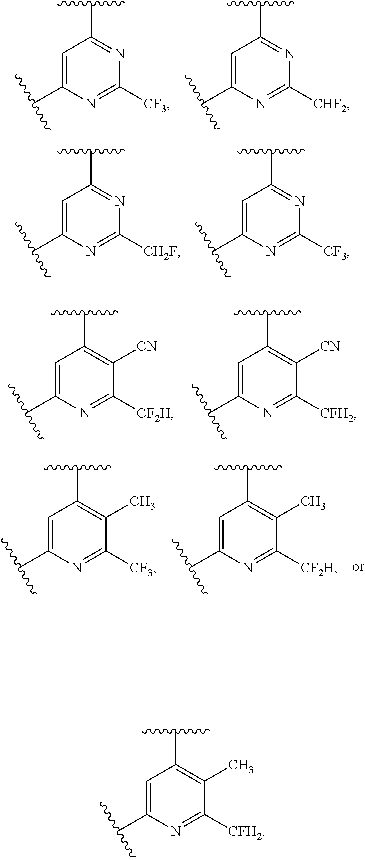

Provided herein as Embodiment B46 is the compound or salt of any one of Embodiments B1-45, wherein q is 0.

Provided herein as Embodiment B47 is the compound or salt of any one of Embodiments B1-45, wherein q is 1.

Provided herein as Embodiment B48 is the compound or salt of any one of Embodiments B1-45, wherein q is 2.

Provided herein as Embodiment B49 is the compound or salt of any one of Embodiments B1-45, wherein r is 0.

Provided herein as Embodiment B50 is the compound or salt of any one of Embodiments B1-45, wherein r is 1.

Provided herein as Embodiment B51 is the compound or salt of any one of Embodiments B1-45, wherein

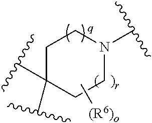

is

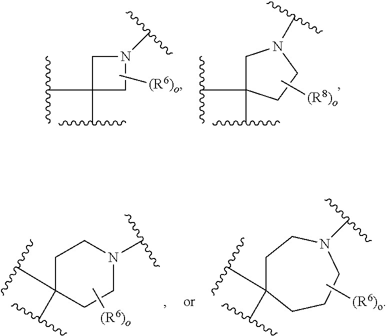

Provided herein as Embodiment B52 is the compound of any one of Embodiments B1-51, wherein o is 0, 1, or 2.

Provided herein as Embodiment B53 is the compound of any one of Embodiments B1-51, wherein o is 3 or 4.

Provided herein as Embodiment B54 is the compound of any one of Embodiments B1-53, wherein at least one R⁶ is Br, Cl, F, CN, CH₃, CH₂F, CHF₂, CF₃, OH, CH₂OH, OCH₃, OCD₃, CH₂OCH₃, CH₂N(CH₃)₂, oxo, ═CH₂, cyclopropyl, cyclobutyl, oxetanyl, tetrahydrofuranyl, spiro-cyclopropyl, spiro-cyclobutyl, spiro-oxetanyl, or spiro-tetrahydrofuranyl; or two vicinal R⁶, together with the atoms to which they are attached, form fused-cyclopropyl, fused-cyclobutyl, or fused-cyclopentyl; or two non-neighboring R⁶ join together to form —CH₂—, —CH₂CH₂—; —CH₂CH₂CH₂—, —CH₂—CH═CH— or —CH₂OCH₂—; wherein each of the cyclopropyl, cyclobutyl, oxetanyl, tetrahydrofuranyl, spiro-cyclopropyl, spiro-cyclobutyl, spiro-oxetanyl, spiro-tetrahydrofuranyl, fused-cyclopropyl, fused-cyclobutyl, and fused-cyclopentyl independently is unsubstituted or substituted with 1-4 substituents, and each substituent independently is halo, C₁₋₃alkyl, C₁₋₃haloalkyl, C₀₋₂alkyleneOH, C₀₋₃alkyleneC₁₋₃alkoxy, or C₀₋₃alkyleneCN.

Provided herein as Embodiment B55 is the compound or salt of Embodiment B54, wherein each substituent independently is F, Cl, OH, OCH₃, OCH₂CH₃, or CN.

Provided herein as Embodiment B56 is the compound or salt of 54 or 55, wherein at least one R⁶ is Br, Cl, F, CN, CH₃, CH_{2F}, CHF₂, CF₃, OH, CH₂OH, OCH₃, OCD₃, CH₂OCH₃, CH₂N(CH₃)₂, oxo, ═CH₂, cyclopropyl, or two vicinal Re, together with the atoms to which they are attached, form fused-cyclopropyl, fused-cyclobutyl, or fused-cyclopentyl, or two non-neighboring R⁶ join together to form —CH₂—, —CH₂CH₂—; —CH₂CH₂CH₂—, —CH₂—CH═CH— or —CH₂OCH₂—; wherein each of the foregoing cycloalkyl groups is unsubstituted or substituted with 1-4 substituents, and each substituent independently is F, Cl, OH, OCH₃, OCH₂CH₃, or CN.

Provided herein as Embodiment B57 is the compound or salt of any one of Embodiments B1-51, wherein o is 0; or o is 1 and R⁶ is H, CH₃, CH OH, or two non-neighboring R⁶ join together to form —CH₂CH₂—.

Provided herein as Embodiment B58 is the compound or salt of any one of Embodiments B1-51, wherein

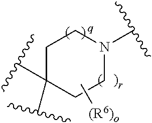

is

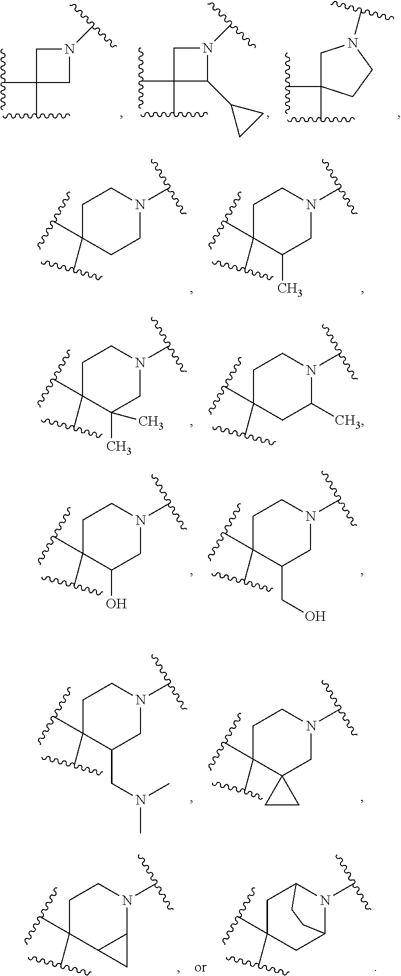

Provided herein as Embodiment B59 is the compound or salt of Embodiment B58, wherein

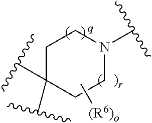

is

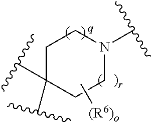

is

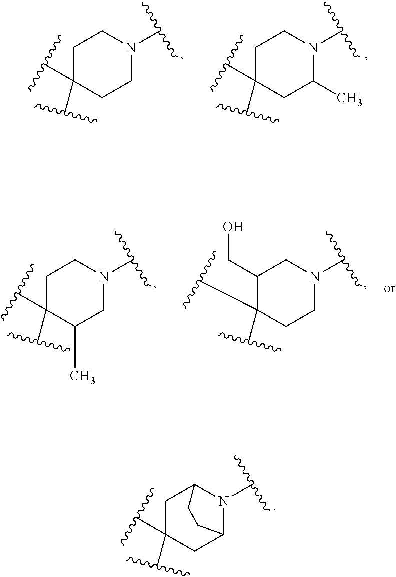

Provided herein as Embodiment B60 is the compound or salt of any one of Embodiments B1-30, wherein the compound is a compound of Formula (IIA), Formula (IIB), or Formula (IIC):

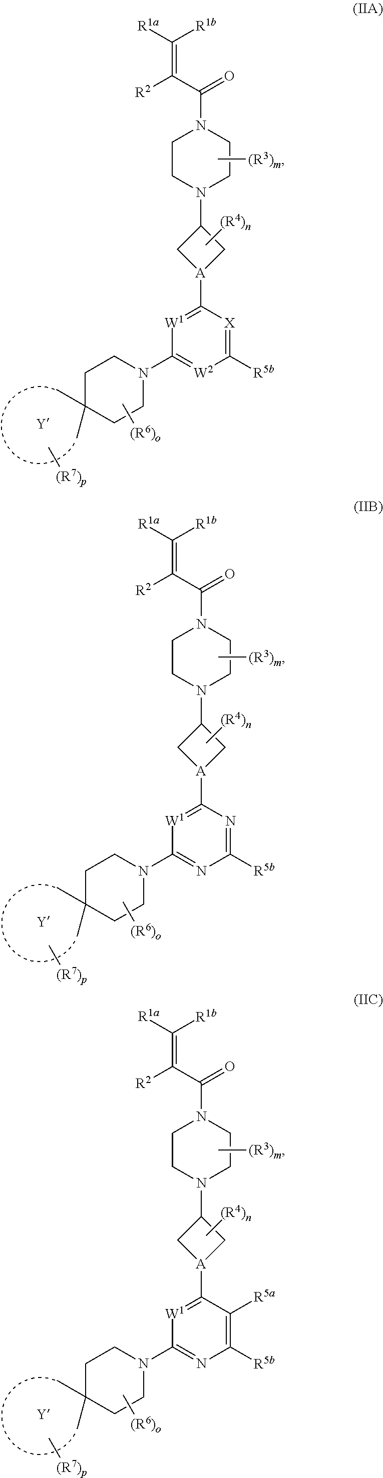

(IIC), or a pharmaceutically acceptable salt of any of the foregoing.

Provided herein as Embodiment B61 is the compound or salt of any one of Embodiments B1-60, wherein Y′ is cyclopropyl, cyclobutyl, cyclopentyl, cyclohexyl, cyclopentenyl, cyclohexenyl, aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, dioxolanyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothiopyranyl, dithianyl, morpholinyl, thiomorpholinyl, dihydropyrrolyl, dihydrofuranyl, dihydrothiopheneyl, dihydroisoxazolyl, tetrahydropyridinyl, dihydropyranyl, or dihydrothipyranyl.

Provided herein as Embodiment B62 is the compound or salt of Embodiment B61, wherein Y′ is cyclopropyl, cyclobutyl, cyclopentyl, cyclopentenyl, oxetanyl, pyrrolidinyl, tetrahydrofuranyl, dioxolanyl, oxazolidinyl, isoxazolidinyl, tetrahydropyranyl, dihydropyrrolyl, dihydrofuranyl or dihydropyranyl.

Provided herein as Embodiment B63 is the compound or salt of any one of Embodiments B1-62, wherein p is 0 or 1.

Provided herein as Embodiment B64 is the compound or salt of any one of Embodiments B1-62, wherein p is 2, 3, or 4.

Provided herein as Embodiment B65 is the compound or salt of any one of Embodiments B1-64, wherein each R′ independently is F, Cl, Br, CN, CH₂CN, CH₃, CH₂CH₃, CH F, CHF₂, CF₃, OH, OCH₃, OCH₂CH₃, CH₂OH, CH₂OCH₃, CH₂CH₂OCH₃, C(═O)OCH₃, oxo, ═CH₂, cyclopropyl,

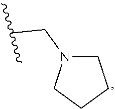 [SMILES: *CN1CCCC1]

oxetanyl, spiro-cyclopropyl, spiro-cyclobutyl, spiro-oxetanyl, spiro-tetrahydrofuranyl, spiro-tetrahydropyranyl; or two vicinal R⁷, together with the atoms to which they are attached, form fused-cyclopropyl, fused-cyclobutyl, fused-oxetanyl, fused-tetrahydrofuranyl, fused-phenyl, fused-thiophenyl, fused-pyrazolyl, fused-thiazolyl, fused-pyridyl, fused-pyridazinyl, fused-pyrazinyl, fused-pyrimidinyl; wherein each of the phenyl, pyrazolyl, pyridyl, pyridazinyl, and pyrimidinyl is unsubstituted or substituted with 1-3 substituents, and each substituent independently is halo. CN, oxo, or C₁₋₃alkyl.

Provided herein as Embodiment B66 is the compound or salt of Embodiment B65, wherein each R⁷ independently is F, Cl, Br, CN, CH₃, CH₂CH₃, CH₂F, OH, CH₂OH, CH₂OCH₃, CH₂CH₂OCH₃, oxo, ═CH₂,

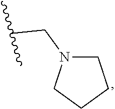 [SMILES: *CN1CCCC1]

oxetanyl, spiro-cyclopropyl, spiro-cyclobutyl, spiro-oxetanyl, or two vicinal R⁷, together with the atoms to which they are attached, form fused-cyclopropyl, fused-phenyl, fused-thiophenyl, fused-pyrazolyl, fused-thiazolyl, fused-pyridyl; wherein each of the phenyl, pyrazolyl, and pyridyl is unsubstituted or substituted with CH₃.

Provided herein as Embodiment B67 is the compound or salt of any one of Embodiments B1-59, wherein

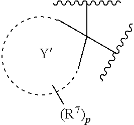

is

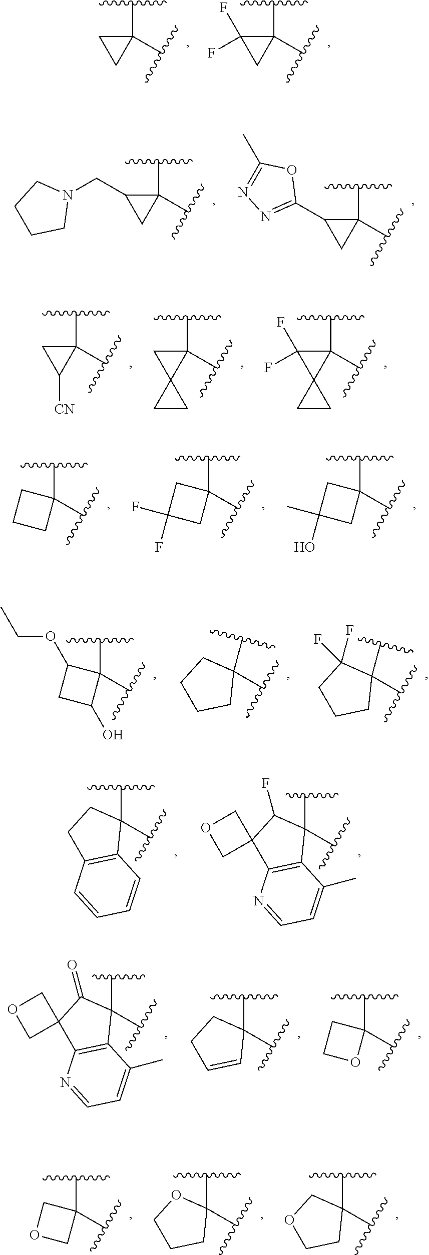

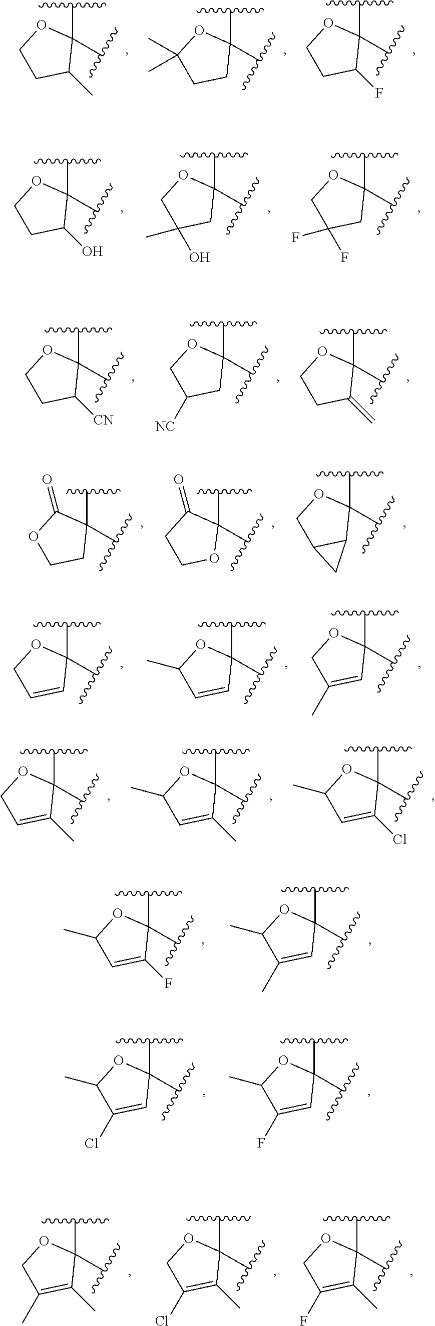

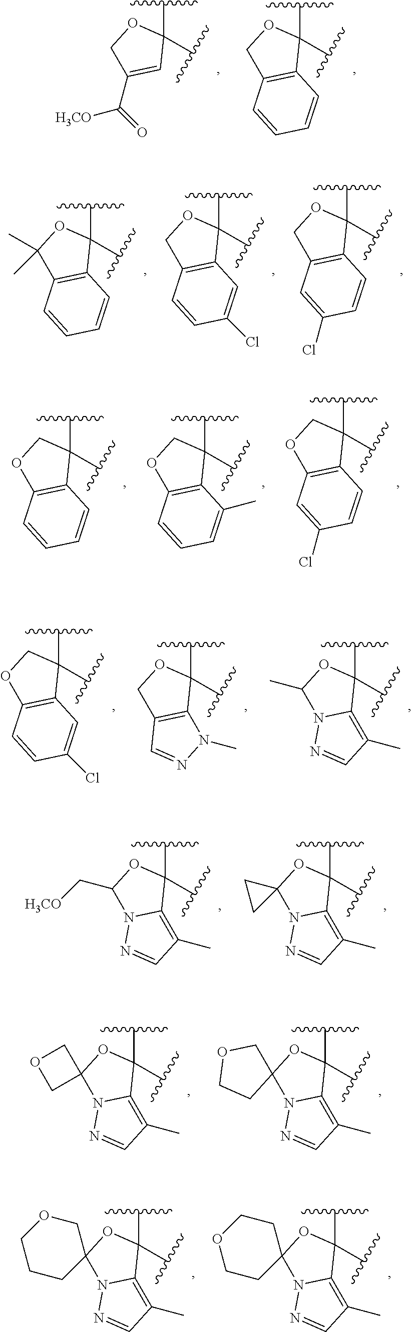

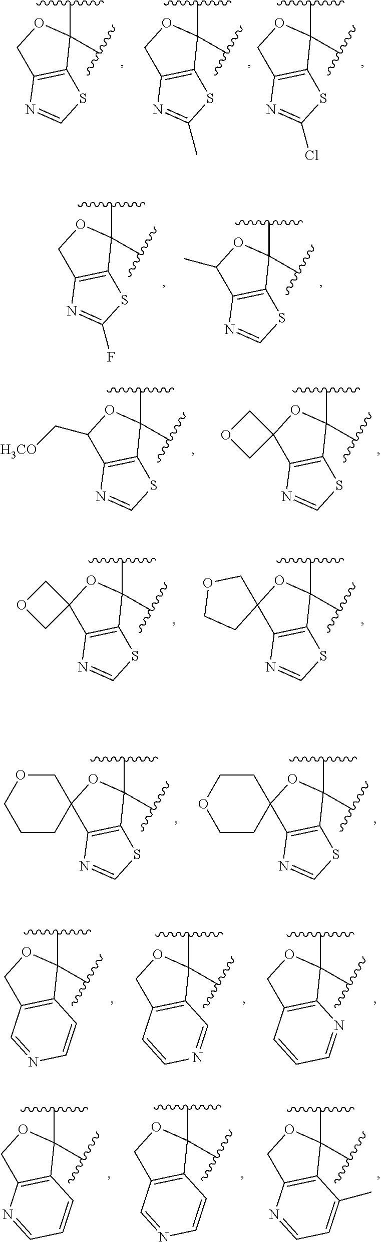

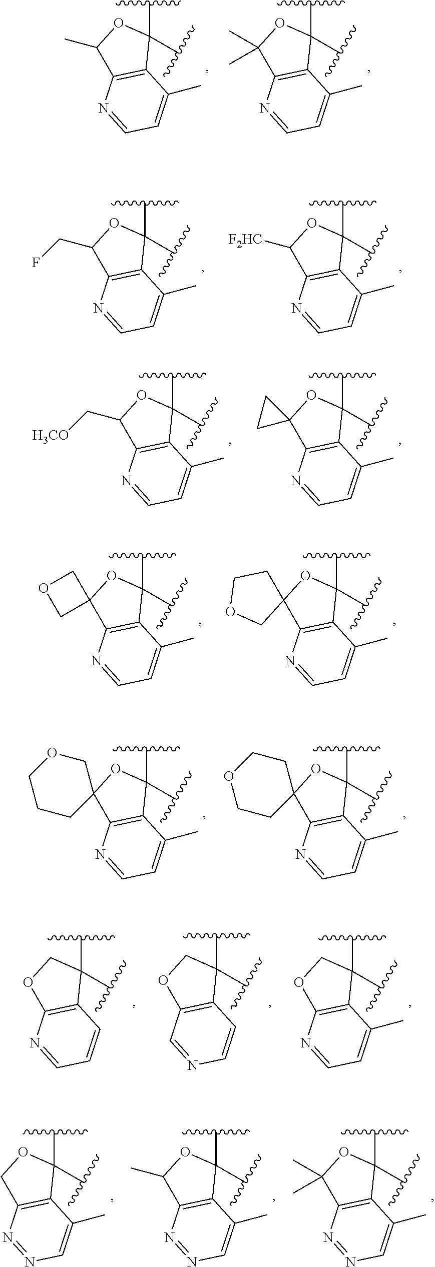

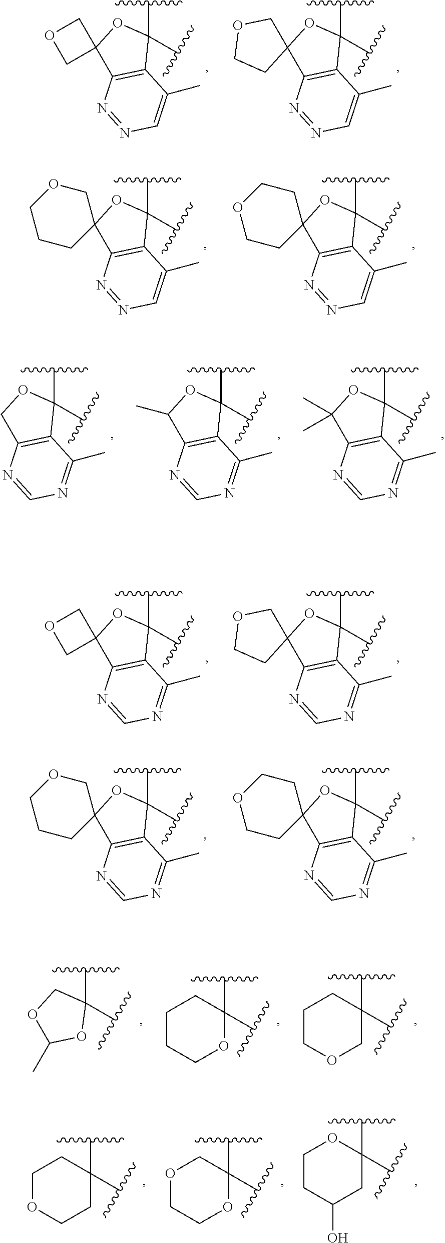

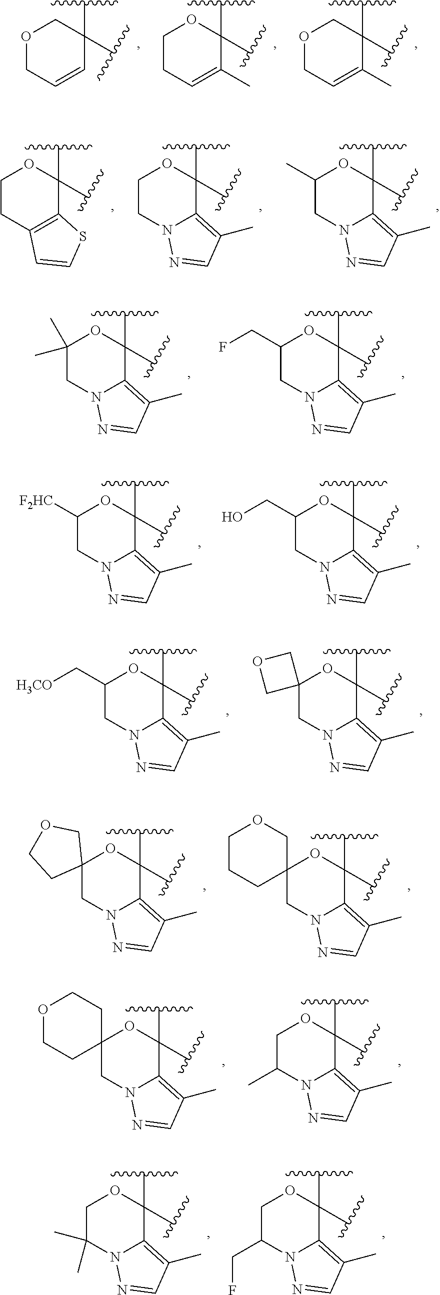

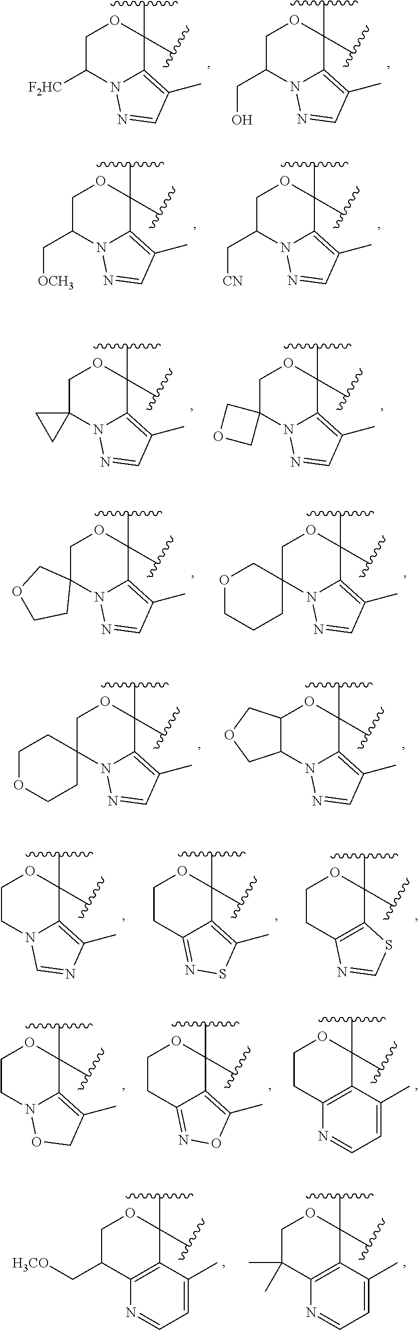

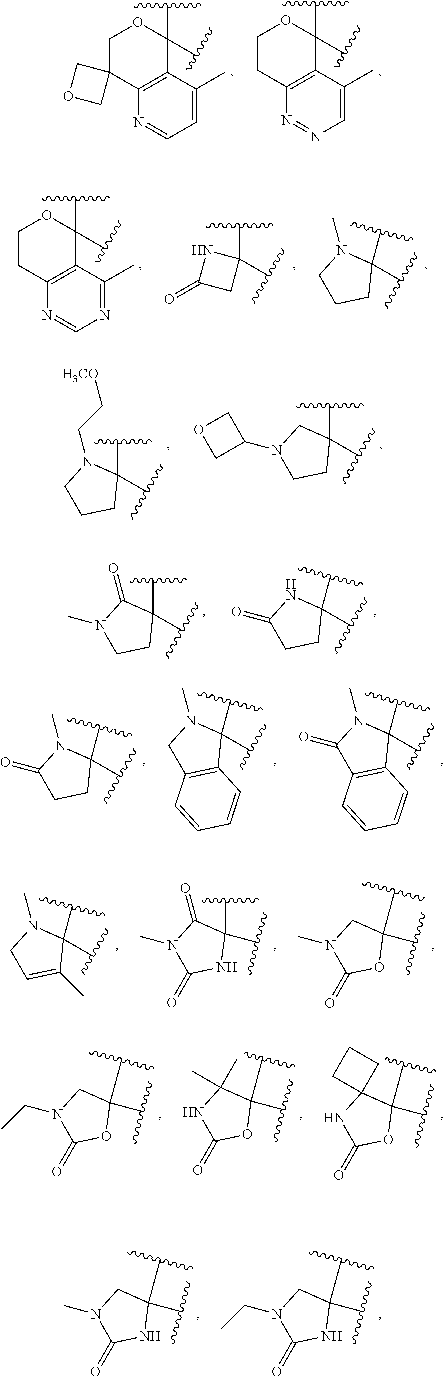

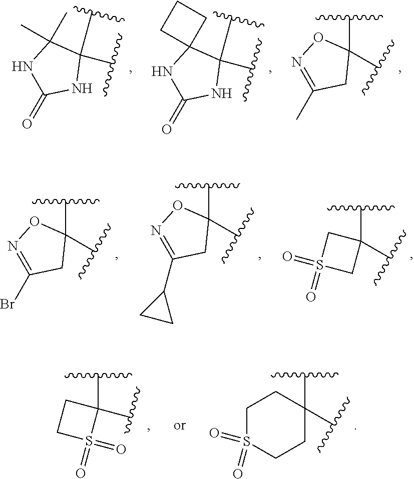

Provided herein as Embodiment B68 is the compound or salt of any one of Embodiments B1-59, wherein

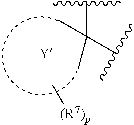

is

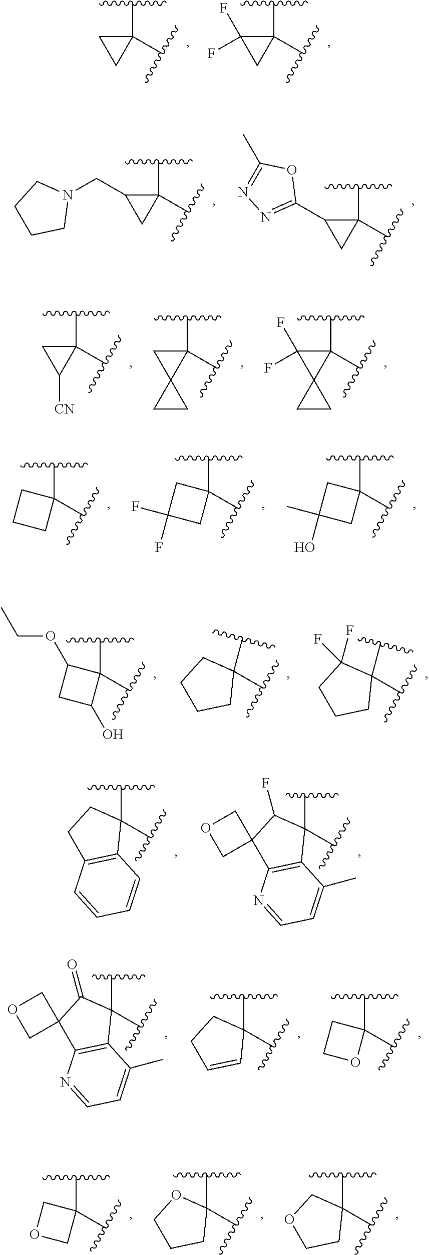

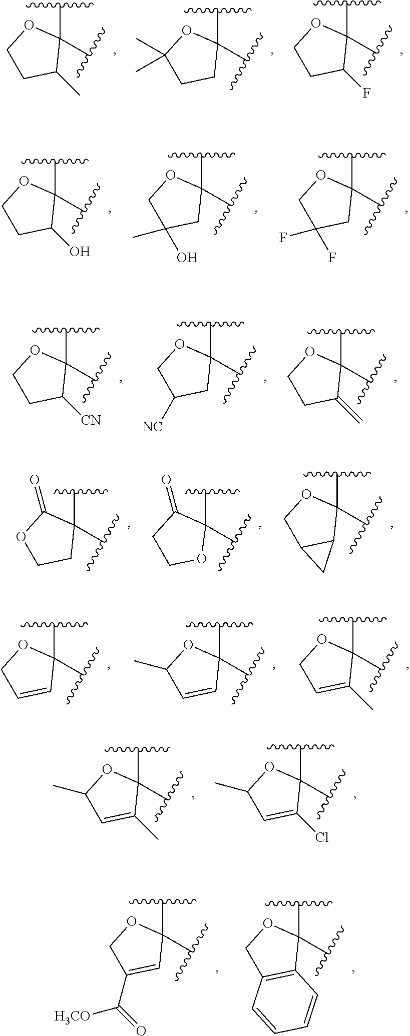

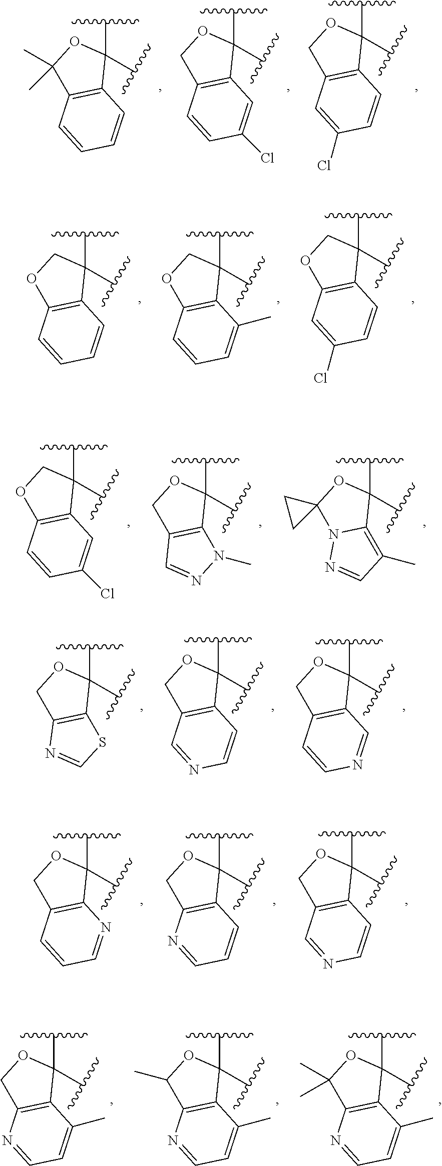

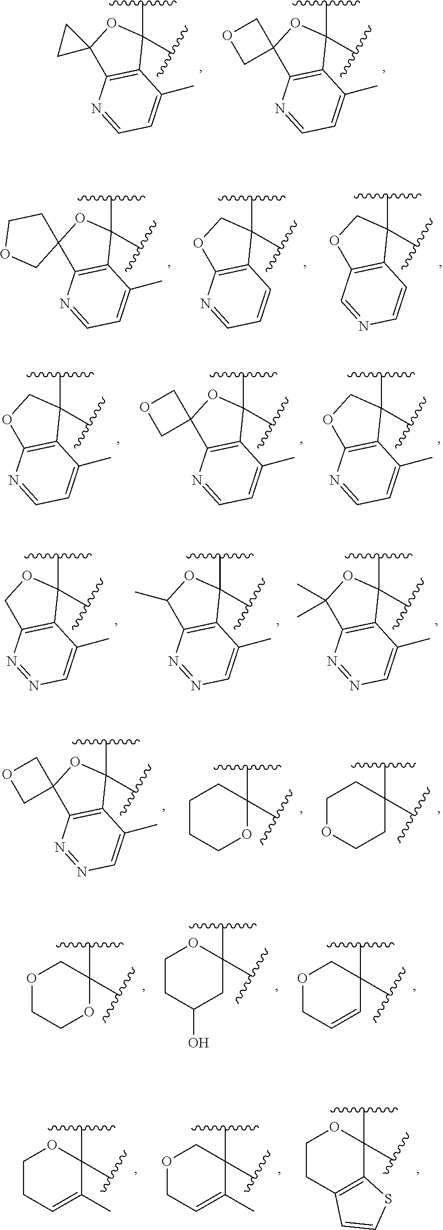

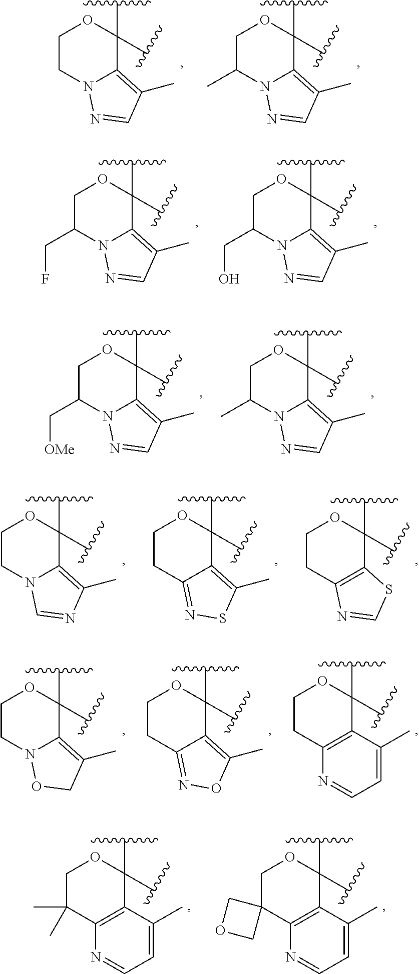

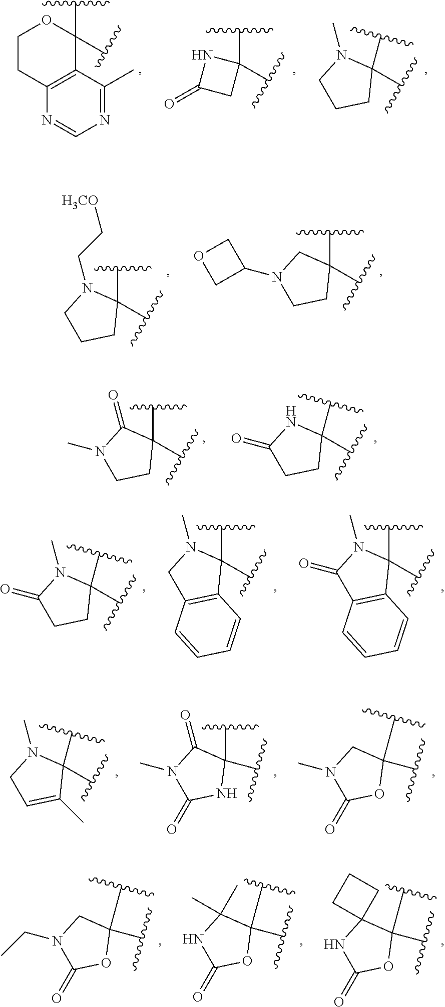

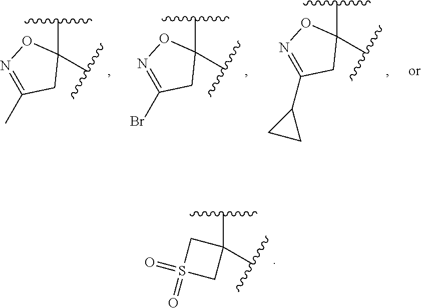

Provided herein as Embodiment B69 is the compound or salt of Embodiment B68, wherein

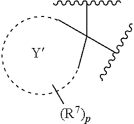

is

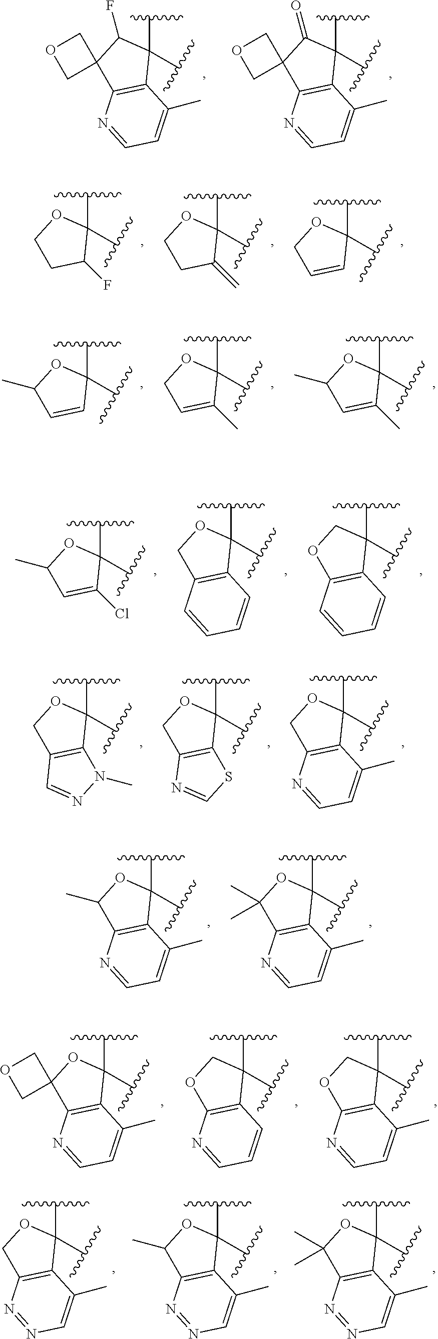

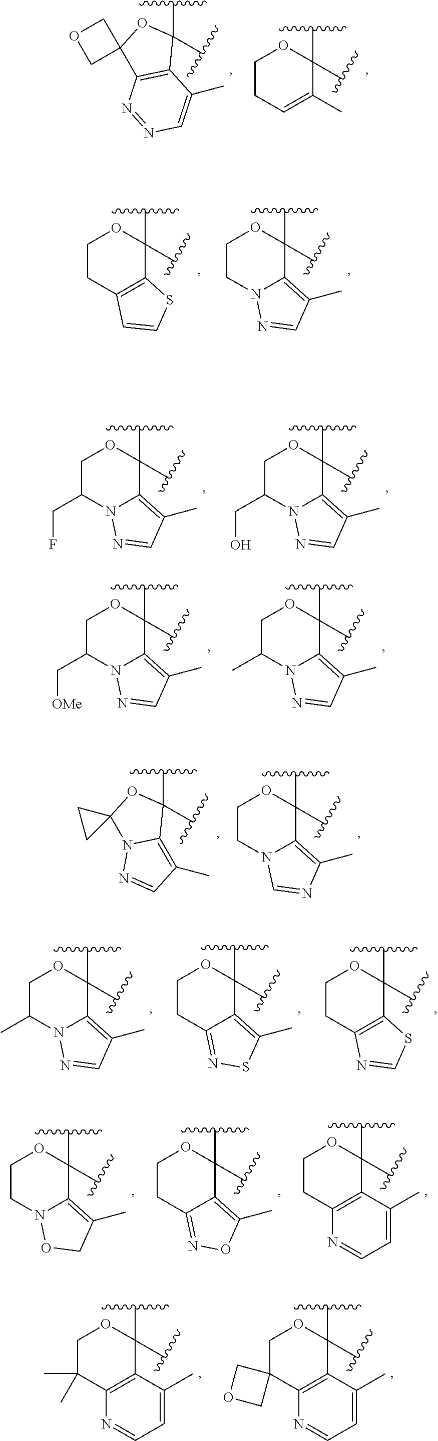

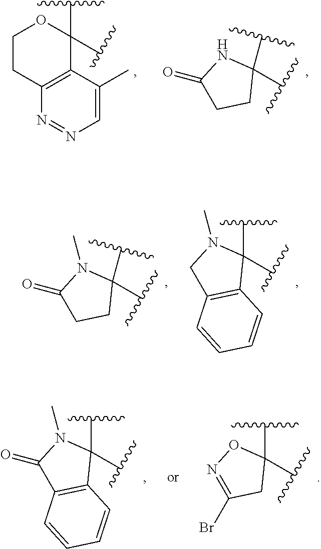

Provided herein as Embodiment B70 is the compound or salt of Embodiment B1, wherein:

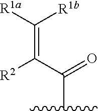

is

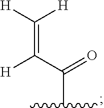 [SMILES: *C(=O)C=C]

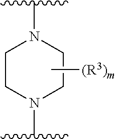

is

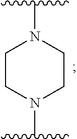 [SMILES: *N1CCN(*)CC1]

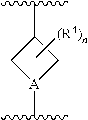

is

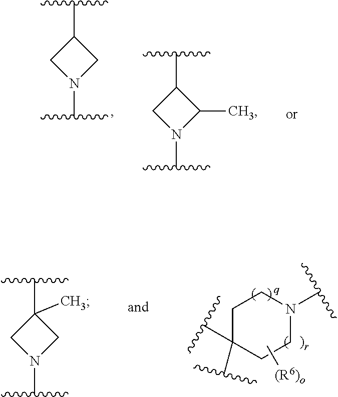

is

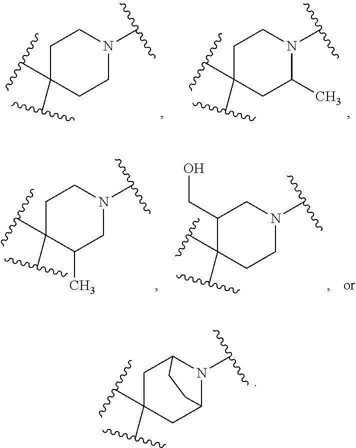

Provided herein as Embodiment B71 is the compound of Embodiment B70, wherein

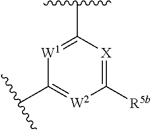

is

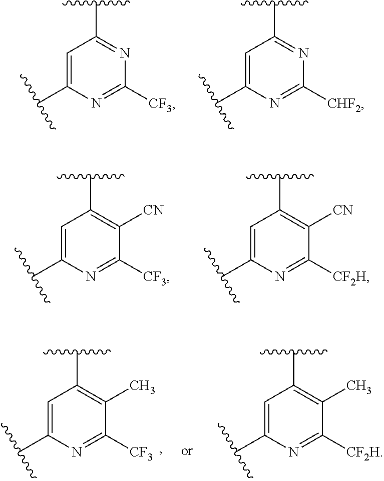

Provided herein as Embodiment B72 is the compound or salt of Embodiment B70 or B71, wherein

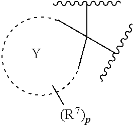

is

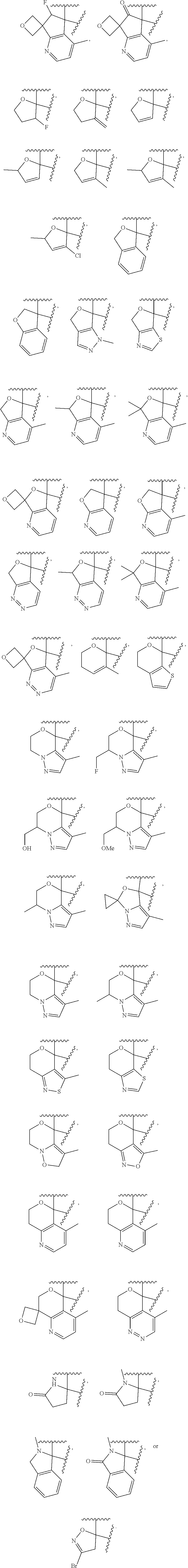

Provided herein as Embodiment B73 is the compound or salt of any one of Embodiments B1-30, wherein q is 1; r is 1; W¹ is CH, C-halo, C—CN, C—C₁₋₃alkyl, C—C₁₋₃haloalkyl, C—C₀₋₃alkyleneOH, or C—C₀₋₃alkylene-C₁₋₄alkoxy; W² is N; and X is N or C—R^{5a}.

Provided herein as Embodiment B74 is the compound of Embodiment B1, wherein the compound is a compound listed in Table A, or a pharmaceutically acceptable salt thereof.

Provided herein as Embodiment B75 is the compound of Embodiment B1, wherein the compound is a compound listed in Table C, or a pharmaceutically acceptable salt thereof.

Provided herein as Embodiment B76 is a pharmaceutical composition comprising the compound or salt of any one of Embodiments B1-75 and a pharmaceutically acceptable excipient.

Provided herein as Embodiment B77 is a method of treating cancer in a subject in need of treatment, the method comprising administering to the subject a therapeutically effective amount of the compound or salt of any one of Embodiments B1-76, or the composition of Embodiment B77.

Provided herein as Embodiment B78 is the method of Embodiment B77, wherein one or more cancer cells express KRAS G12C mutant protein.

Provided herein as Embodiment B79 is the method of Embodiment B77 or B78, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Provided herein as Embodiment B80 is the method of Embodiment B79, wherein the cancer is non-small cell lung cancer, colorectal cancer, pancreatic cancer, or solid tumor.

Provided herein as Embodiment B81 is the method according to any one of Embodiments B77-80, wherein the subject has a cancer that was determined to have one or more cells expressing the KRAS G12C mutant protein prior to administration of the compound, salt, or pharmaceutical composition.

Provided herein as Embodiment B82 is the method according to any one of Embodiments B77-81, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor. FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

Provided herein as Embodiment B83 is the compound or salt of any one of Embodiments B1-75, or the composition of Embodiment B76 for use as a medicament.

Provided herein as Embodiment B84 is the compound or salt of any one of Embodiments B1-75, or the composition of Embodiment B76 for use in treating cancer.

Provided herein as Embodiment B85 is the compound or salt of any one of Embodiments B1-75 or the pharmaceutical composition of Embodiment B76 for use in treating cancer, wherein one or more cancer cells express KRAS G12C mutant protein.

Provided herein as Embodiment B86 is the compound or salt of Embodiment B84 or B85, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment B87 is the compound or salt of Embodiment B86, wherein the cancer is non-small cell lung cancer, colorectal cancer, pancreatic cancer, or solid tumor.

Provided herein as Embodiment B88 is a use of a compound or salt of any one of Embodiments B1-75 or the pharmaceutical composition of Embodiment B76 for the manufacture of a medicament for the treatment of cancer.

Provided herein as Embodiment B89 is a use of a compound or salt of any one of Embodiments B1-75 or the pharmaceutical composition of Embodiment B76 in the preparation of a medicament for treating cancer, wherein one or more cancer cells express KRASG12C mutant protein.

Provided herein as Embodiment B90 is the use of Embodiment B88 or B89, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Provided herein as Embodiment B91 is the use of Embodiment B90, wherein the cancer is non-small cell lung cancer, colorectal cancer, pancreatic cancer, or solid tumor.

Provided herein as Embodiment B92 is a compound of Formula (B):

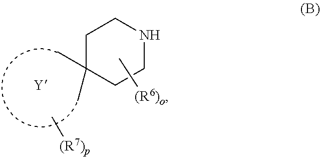

or a nitrogen-protected analog, or a pharmaceutically acceptable salt of any of the foregoing, wherein:
o is 0, 1, 2, 3, or 4;
p is 0, 1, 2, 3, 4, or 5;
each R⁶ s halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N. O, and S; or two non-neighboring Rejoin together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₃alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

Provided herein as Embodiment B93 is a method of preparing the compound or salt of any one of Embodiments B1-75, comprising converting a compound of Embodiment B92, a protected analog of any of the foregoing, or a pharmaceutically acceptable salt of any of the foregoing, into a compound of any one of Embodiments B1-75.

### Additional Embodiments

Provided herein as Embodiment A1 is a compound of Formula (II):

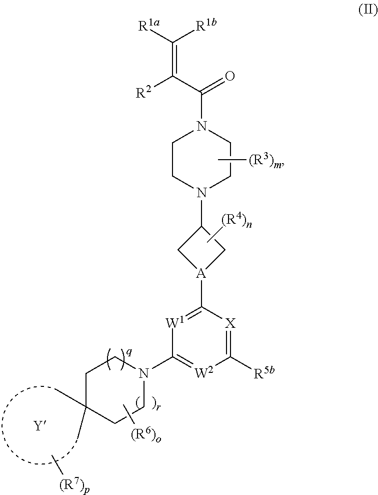

or a pharmaceutically acceptable salt thereof,
wherein:
m is 0, 1, 2, 3, or 4;
n is 0, 1, or 2;
o is 0, 1, 2, 3, or 4;
p is 0, 1, 2, 3, 4, or 5;
q is 0, 1, or 2;
r is 0 or 1;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃haloalkyl, C—C₀₋₃alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkyleneOH, or C₀₋₃alkyleneC₁₋₄alkoxy;
X is N or C—R^{5a};
Y′ is C₃₋₇cycloalkyl, C₄₋₇cycloalkenyl, heterocycloalkyl having 3 to 7 total ring atoms and 1-3 heteroatoms independently selected from N, O, and S, or heterocycloalkenyl having 5 to 7 total ring atoms and 1-3 heteroatoms independently selected from N, O, and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form

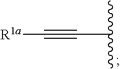

each R³ independently is C₁₋₃alkyl, C₁₋₃haloalkyl,

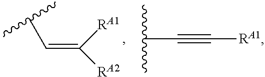

C₀₋₃alkyleneCN, C₀₋₃alkyleneOH, C₀₋₃alkylene-C₁₋₃alkoxy, oxo, spiro-C₃₋₇cycloalkyl, spiro-C₄₋₇cycloalkenyl, spiro-heterocycloalkyl having 3-7 total ring atoms and 1 or 2 heteroatoms independently selected from N, O, and S, or spiro-heterocycloalkenyl having 4-7 total ring atoms and 1 or 2 heteroatoms independently selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms independently selected from N, O, and S, or fused-heterocycloalkenyl having 4-7 total ring atoms and 1 or 2 heteroatoms independently selected from N, O, and S;
each of R^{A1} and R^{A2} independently is H, C₁₋₃alkyl, C₁₋₃haloalkyl, or C₃₋₅cycloalkyl;
each R⁴ independently is C₁₋₃alkyl, C₁₋₃haloalkyl, C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 heteroatoms independently selected from N, O and S;
R^{5a} is H, CN, halo, C₁₋₃alkyl, C₁₋₃haloalkyl, C₂₋₆alkenyl, C₂₋₆alkynyl, C₀₋₃alkyleneOH, C₀₋₃alkylene-C₁₋₃alkoxy, or C₃₋₅cycloalkyl;
R^{5b} is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms independently selected from N, O, and S, heterocycloalkenyl having 5-7 total ring atoms and 1-3 heteroatoms independently selected from N, O, and S, or R^{5a} and R^{5b}, together with the atoms to which they are attached, form C₃₋₇ cycloalkyl;
wherein each of the foregoing C₁₋₆alkyl, C₂₋₄alkenyl, C₂₋₄alkynyl, C₃₋₇cycloalkyl, C₅₋₇cycloalkenyl, heterocycloalkyl, and heterocycloalkenyl independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl;
each R⁶ independently is halo, CN, C₁₋₃alkyl, C₂₋₃alkonyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal Re, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

Provided herein as Embodiment A2 is the compound or salt of Embodiment A1, wherein at least one of R^{1a}, R^{1b}, and R² is H or D.

Provided herein as Embodiment A3 is the compound or salt of Embodiment A2, wherein each of R^{1a}, R^{1b}, and R² independently is H or D.

Provided herein as Embodiment A4 is the compound or salt of Embodiment A3, wherein each of R^{1a}, R^{1b}, and R² is H.

Provided herein as Embodiment A5 is the compound or salt of Embodiment A3, wherein each of R^{1a}, R^{1b}, and R² is D.

Provided herein as Embodiment A6 is the compound or salt of Embodiment A1 or A2, wherein at least one of R^{1a}, R^{1b}, and R² is halo.

Provided herein as Embodiment A7 is the compound or salt of Embodiment A6, wherein R^{1a} is halo and each of R^{1b} and R² is H.

Provided herein as Embodiment A8 is the compound or salt of Embodiment A6 or A7, wherein each halo independently is Br, Cl, or F.

Provided herein as Embodiment A9 is the compound or salt of Embodiment A1 or A2, wherein at least one of R^{1a}, R^{1b}, and R² is C₁₋₄alkyl or C₁₋₄haloalkyl.

Provided herein as Embodiment A10 is the compound or salt of Embodiment A9, wherein at least one of R^{1a}, R^{1b}, and R² is CH₃, CH₂F, CHF₂, or CF₃.

Provided herein as Embodiment A11 is the compound or salt of Embodiment A1 or A2, wherein at least one of R^{1a}, R^{1b}, and R² is C₁₋₂alkylene-OH, C₀₋₂alkylene-C₁₋₄alkoxy, C₀₋₂alkylene-C₁₋₄haloalkoxy, C₀₋₂alkylene-CN, or C₀₋₂alkylene-N(R^{N1})₂.

Provided herein as Embodiment A12 is the compound or salt of Embodiment A11, wherein each R^{N1} independently is H or CH₃.

Provided herein as Embodiment A13 is the compound or salt of Embodiment A12, wherein each R^{N1} is H.

Provided herein as Embodiment A14 is the compound or salt of Embodiment A11 or A12, wherein at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂.

Provided herein as Embodiment A15 is the compound or salt of Embodiment A1 or A2, wherein at least one of R^{1a}, R^{1b}, and R² is C₁₋₂alkylene-heterocycloalkyl having 3-6 total ring atoms and 1 or 2 heteroatoms independently selected from N, O, and S.

Provided herein as Embodiment A16 is the compound or salt of Embodiment A15, wherein the heterocycloalkyl is aziridinyl, azetidinyl, pyrrolidinyl, piperidinyl, or morpholinyl.

Provided herein as Embodiment A17 is the compound or salt of Embodiment A15 or A17, wherein at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl.

Provided herein as Embodiment A18 is the compound or salt of Embodiment A1 or A2, wherein R^{1b} and R², together with the carbon atoms to which they are attached, form

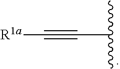

Provided herein as Embodiment A19 is the compound or salt of Embodiment A1 or A2, wherein

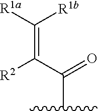

is

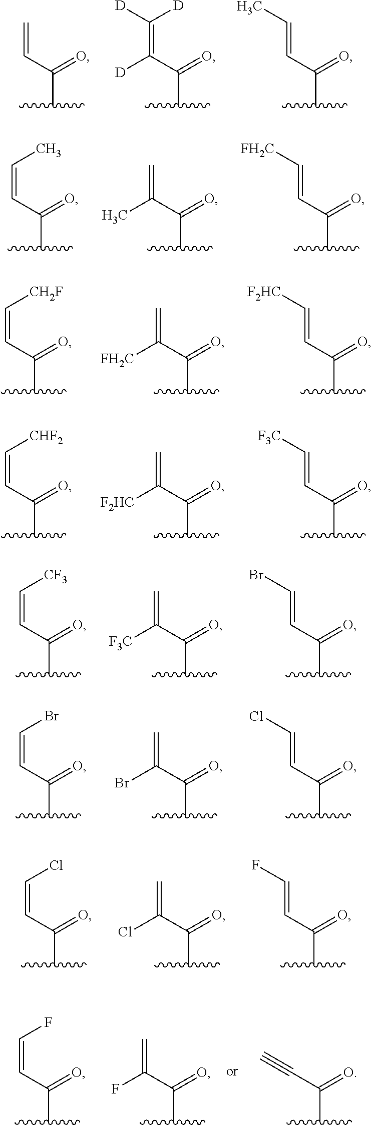

Provided herein as Embodiment A20 is the compound or salt of Embodiment A19, wherein

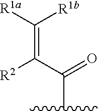

is

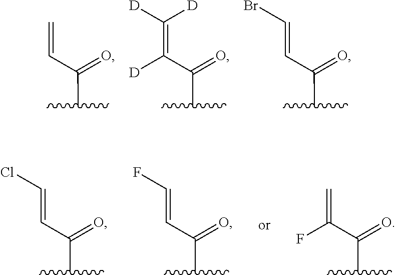

Provided herein as Embodiment A21 is the compound or salt of Embodiment A20, wherein

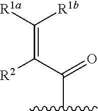

is

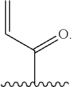 [SMILES: *C(=O)C=C]

Provided herein as Embodiment A22 is the compound or salt of any one of Embodiments A1-A21, wherein m is 0.

Provided herein as Embodiment A23 is the compound or salt of any one of Embodiments A1-A21, wherein m is 1.

Provided herein as Embodiment A24 is the compound or salt of any one of Embodiments A1-A21, wherein m is 2.

Provided herein as Embodiment A25 is the compound or salt of any one of Embodiments A1-A21, wherein m is 3.

Provided herein as Embodiment A26 is the compound or salt of any one of Embodiments A1-A21, wherein m is 4.

Provided herein as Embodiment A27 is the compound or salt of any one of Embodiments A23-A26, wherein

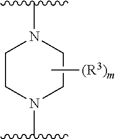

is deuterated.

Provided herein as Embodiment A28 is the compound or salt of Embodiment A27, wherein

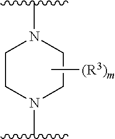

is

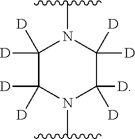

Provided herein as Embodiment A29 is the compound or salt of any one of Embodiments A23-A26, wherein at least one R³ is C₁₋₃alkyl or C₁₋₃haloalkyl.

Provided herein as Embodiment A30 is the compound or salt of Embodiment A29, wherein at least one R³ is CH₃, CH₂CH₃, CF₃, CHF₂, or CH₂F.

Provided herein as Embodiment A31 is the compound or salt of Embodiment A30, wherein at least one R³ is CH₃.

Provided herein as Embodiment A32 is the compound or salt of any one of Embodiments A23-A26, wherein at least one R³ is

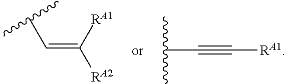

Provided herein as Embodiment A33 is the compound or salt of Embodiment A32, wherein each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl.

Provided herein as Embodiment A34 is the compound or salt of Embodiment A32 or A33, wherein at least one R³ is

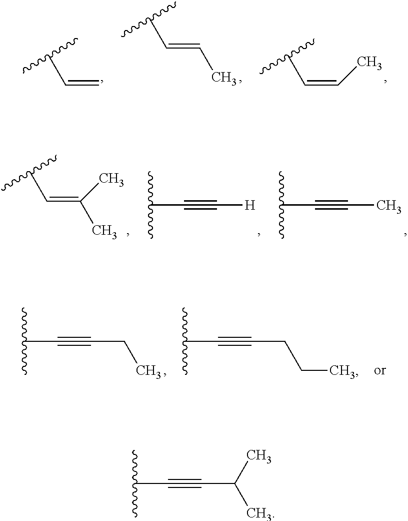

Provided herein as Embodiment A35 is the compound or salt of any one of Embodiments A23-A26, wherein at least one R³ is C₀₋₃alkyleneCN.

Provided herein as Embodiment A36 is the compound or salt of Embodiment A35, wherein at least one R³ is CN or CH₂CN.

Provided herein as Embodiment A37 is the compound or salt of any one of Embodiments A23-A26, wherein at least one R³ is C₀₋₃alkyleneOH or C₀₋₃alkylene-C₁₋₃alkoxy.

Provided herein as Embodiment A38 is the compound or salt of Embodiment A37, wherein at least one R³ is OH, CH₂OH, CH₂CH₂OH, OCH₃, CH₂OCH₃, or CH₂CH₂OCH₃.

Provided herein as Embodiment A39 is the compound or salt of any one of Embodiments A23-A26, wherein at least one R³ is oxo.

Provided herein as Embodiment A40 is the compound or salt of any one of Embodiments A23-A26, wherein at least one R³ is spiro-C₃₋₇cycloalkyl or spiro-heterocycloalkyl having 3-7 total ring atoms and 1 or 2 heteroatoms independently selected from N, O and S.

Provided herein as Embodiment A41 is the compound or salt of Embodiment A40, wherein at least one R³ is spiro-cyclopropyl, spiro-cyclobutyl, spiro-oxetanyl, or spiro-tetrahydrofuranyl.

Provided herein as Embodiment A42 is the compound or salt of any one of Embodiments A23-A26, wherein at least one R³ is spiro-C₄₋₇cycloalkenyl or spiro-heterocycloalkenyl having 4-7 total ring atoms and 1 or 2 heteroatoms independently selected from N, O and S.

Provided herein as Embodiment A43 is the compound or salt of any one of Embodiments A24-A26, wherein two vicinal R³, together with the atoms to which they are attached, form fused-C₃₋₇cycloalkyl or fused-heterocycloalkyl having 3-7 total ring atoms and 1 or 2 heteroatoms independently selected from N, O and S.

Provided herein as Embodiment A44 is the compound or salt of Embodiment A43, wherein two vicinal R³, together with the atoms to which they are attached, form fused-cyclopropyl or fused-cyclobutyl.

Provided herein as Embodiment A45 is the compound or salt of any one of Embodiment A24-A26, wherein two vicinal R³, together with the atoms to which they are attached, form fused-Ca cycloalkenyl or fused-heterocycloalkenyl having 4-7 total ring atoms and 1 or 2 heteroatoms independently selected from N, O and S.

Provided herein as Embodiment A46 is the compound or salt of any one of Embodiments A23-A26, wherein each R³ independently is CH₃, CH₂CH₃, CF₃, CHF₂, CH₂F,

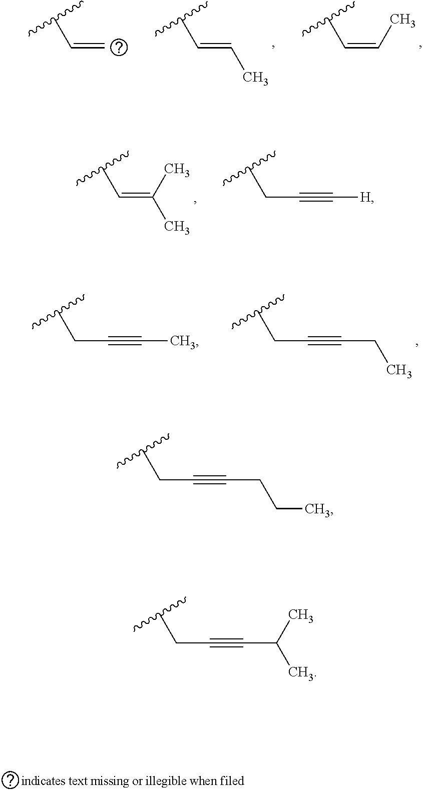

CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl.

Provided herein as Embodiment A47 is the compound or salt of any one of Embodiments A1-A21, wherein m is 0; or m is 1 and R³ is CH₃, CH₂F, CHF₂, CF₃, CN, CH₂CN, CH₂OH, CH₂OCH₃, or spiro-oxetanyl.

Provided herein as Embodiment A48 is the compound or salt of Embodiment A47, wherein m is 0; or m is 1 and R³ is CH₃.

Provided herein as Embodiment A49 is the compound or salt of any one of Embodiments A1-A21, wherein

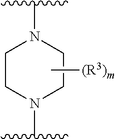

is

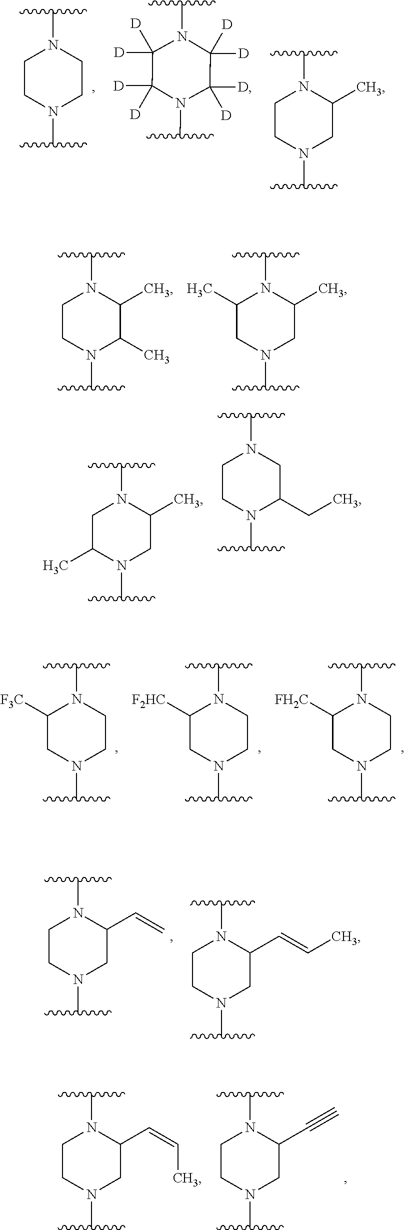

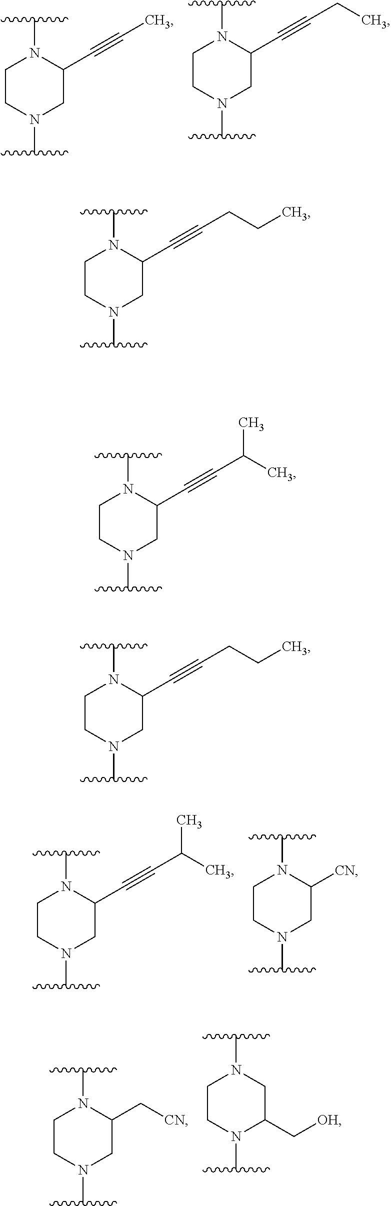

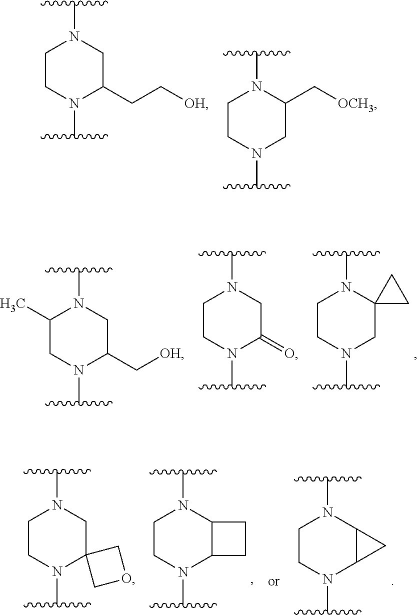

Provided herein as Embodiment A50 is the compound or salt of Embodiment A49, wherein

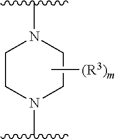

is

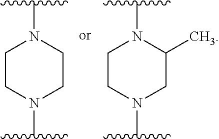

Provided herein as Embodiment A51 is the compound or salt of Embodiment A50, wherein

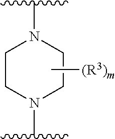

is

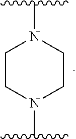 [SMILES: *N1CCN(*)CC1]

Provided herein as Embodiment A52 is the compound or salt of any one of Embodiments A1-A51, wherein A is N.

Provided herein as Embodiment A53 is the compound or salt of any one of Embodiments A1-A51, wherein A is CH, C-halo, C—CN, C—C₁₋₃alkyl, C—C₁₋₃haloalkyl, C—C₀₋₃alkyleneOH, or C—C₀₋₃alkylene-C₁₋₄alkoxy.

Provided herein as Embodiment A54 is the compound or salt of Embodiment A53, wherein A is CH.

Provided herein as Embodiment A55 is the compound or salt of Embodiment A53, wherein A is C—F, C—Cl, or C—CN.

Provided herein as Embodiment A56 is the compound or salt of Embodiment A53, wherein A is C—C₁₋₃alkyl.

Provided herein as Embodiment A57 is the compound or salt of Embodiment A56, wherein A is C—CH₃.

Provided herein as Embodiment A58 is the compound or salt of Embodiment A53, wherein A is C—C₁₋₃haloalkyl.

Provided herein as Embodiment A59 is the compound or salt of Embodiment A58, wherein A is C—CH₂F, C—CHF₂, or C—CF₃.

Provided herein as Embodiment A60 is the compound or salt of Embodiment A53, wherein A is C—C₀₋₃alkyleneOH or C—C₀₋₃alkylene-C₁₋₄alkoxy.

Provided herein as Embodiment A61 is the compound or salt of Embodiment A60, wherein A is C—OH, C—CH₂OH, C—OCH₃, or C—CH₂OCH₃.

Provided herein as Embodiment A62 is the compound or salt of Embodiment A53, wherein A is CH, C—F, C—Cl, C—CN, C—CH₃, C—CH₂F, C—CHF₂, C—CF₃, C—OH, C—CH₂OH, C—OCH₃, or C—CH₂OCH₃.

Provided herein as Embodiment A63 is the compound or salt of any one of Embodiments A1-A62, wherein n is 0.

Provided herein as Embodiment A64 is the compound or salt of any one of Embodiments A1-A62, wherein n is 1.

Provided herein as Embodiment A65 is the compound or salt of any one of Embodiments A1-A62, wherein n is 2.

Provided herein as Embodiment A66 is the compound or salt of Embodiment A64 or A65, wherein at least one R₄ is C₁₋₃alkyl or C₁₋₃haloalkyl.

Provided herein as Embodiment A67 is the compound or salt of Embodiment A66, wherein at least one R⁴ is CH₃, CH₂CH₃, CH₂CH₂CH₃, CH(CH₃)₂, CH₂F, CHF₂, or CF₃.

Provided herein as Embodiment A68 is the compound or salt of Embodiment A67, wherein at least one R⁴ is CH₃.

Provided herein as Embodiment A69 is the compound or salt of Embodiment A64 or A65, wherein at least one R⁴ is C₀₋₃alkyleneCN.

Provided herein as Embodiment A70 is the compound or salt of Embodiment A69, wherein at least one R⁴ is CN or CH₂CN.

Provided herein as Embodiment A71 is the compound or salt of Embodiment A64 or A65, wherein at least one R⁴ is C₁₋₃alkyleneOH or C₁₋₃alkylene-C₁₋₃alkoxy.

Provided herein as Embodiment A72 is the compound or salt of Embodiment A71, wherein at least one R⁴ is CH₂OH, CH₂CH₂OH, CH₂OCH₃, or CH₂CH₂OCH₃.

Provided herein as Embodiment A73 is the compound or salt of Embodiment A64 or A65, wherein at least one R⁴ is oxo.

Provided herein as Embodiment A74 is the compound or salt of Embodiment A64 or A65, wherein at least one R⁴ is spiro-C₃₋₇cycloalkyl or spiro-heterocycloalkyl having 4-7 total ring atoms and 1 or 2 heteroatoms independently selected from N, O and S.

Provided herein as Embodiment A75 is the compound or salt of Embodiment A74, wherein at least one R⁴ is spiro-cyclopropyl, spiro-cyclobutyl, or spiro-oxetanyl.

Provided herein as Embodiment A76 is the compound or salt of Embodiment A64 or A65, wherein each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl.

Provided herein as Embodiment A77 is the compound or salt of Embodiment A52, wherein

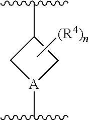

is

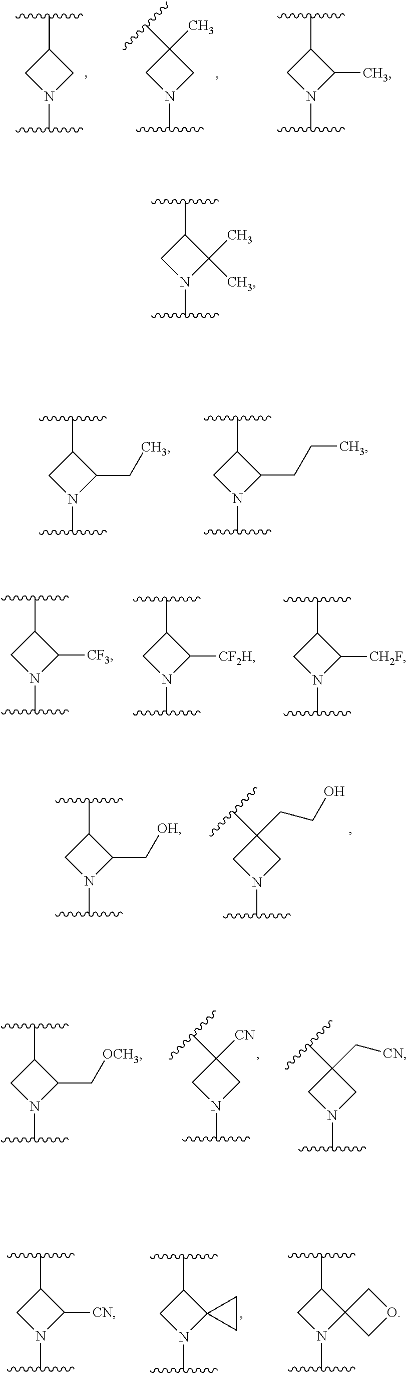

Provided herein as Embodiment A78 is the compound or salt of Embodiment A77, wherein

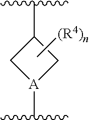

is

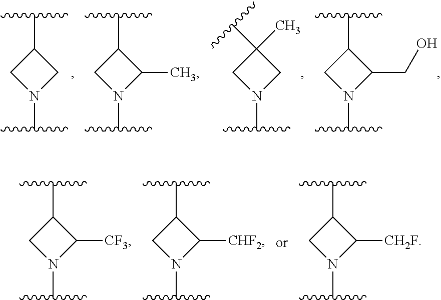

Provided herein as Embodiment A79 is the compound or salt of Embodiment A78, wherein

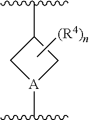

is

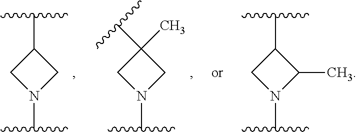

Provided herein as Embodiment A80 is the compound or salt of Embodiment A79, wherein

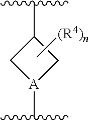

is

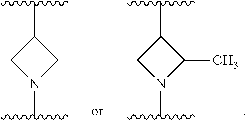

Provided herein as Embodiment A81 is the compound or salt of Embodiment A53, wherein

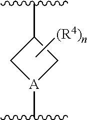

is

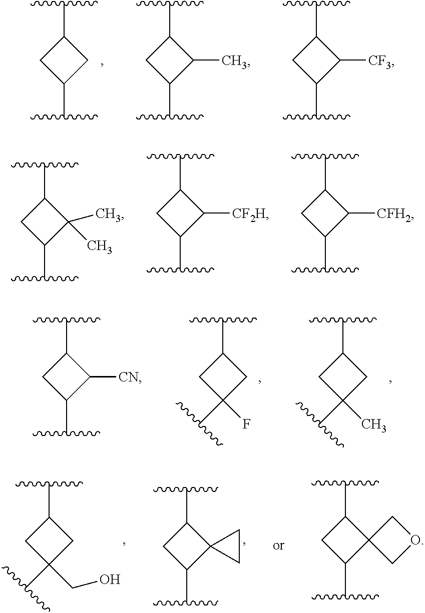

Provided herein as Embodiment A82 is the compound or salt of any one of Embodiments A1-A81, wherein W¹ is N.

Provided herein as Embodiment A83 is the compound or salt of any one of Embodiments A1-A81, wherein W¹ is CH.

Provided herein as Embodiment A84 is the compound or salt of any one of Embodiments A1-A81, wherein W¹ is C—F, C—Cl, or C—CN.

Provided herein as Embodiment A85 is the compound or salt of any one of Embodiments A1-A81, wherein W¹ is C—C₁₋₃alkyl or C—C₁₋₃haloalkyl.

Provided herein as Embodiment A86 is the compound or salt of Embodiment A85, wherein W¹ is C—CH₃, C—CH—CH₃, C—CH₂F, C—CHF₂, or C—CF₃.

Provided herein as Embodiment A87 is the compound or salt of any one of Embodiments A1-A85, wherein W₁ is C—C₂₋₃alkenyl or C—C₂₋₃alkynyl, and each of the alkenyl and alkynyl is unsubstituted or substituted with 1-3 substituents, and each substituent independently is halo, C₁₋₃haloalkyl, C₀₋₃alkyleneOH, or C₀₋₃alkyleneC₁₋₄alkoxy.

Provided herein as Embodiment A88 is the compound or salt of Embodiment A87, wherein W¹ is C—CH═CH₂, C—C(OH)═CH₂, C—CH═CH(OH), or C—CCH.

Provided herein as Embodiment A89 is the compound or salt of any one of Embodiments A1-A81, wherein W¹ is C—C₀₋₃alkyleneOH or C—C₀₋₃alkylene-C₁₋₄alkoxy.

Provided herein as Embodiment A90 is the compound or salt of Embodiment A89, wherein W¹ is C—OH, C—CH₂OH, C—OCH₃, or C—CH₂OCH₃.

Provided herein as Embodiment A91 is the compound or salt of any one of Embodiments A1-A81, wherein W¹ is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃.

Provided herein as Embodiment A92 is the compound or salt of any one of Embodiments A1-A91, wherein W₂ is N.

Provided herein as Embodiment A93 is the compound or salt of any one of Embodiments A1-A91, wherein W² is CH.

Provided herein as Embodiment A94 is the compound or salt of any one of Embodiments A1-A91, wherein W² is C—F, C—Cl, or C—CN.

Provided herein as Embodiment A95 is the compound or salt of any one of Embodiments A1-A91, wherein W₂ is C—C₁₋₃alkyl or C—C₁₋₃haloalkyl.

Provided herein as Embodiment A96 is the compound or salt of Embodiment A95, wherein W² is C—CH₃, C—CH₂CH₃, C—CH₂F, C—CHF₂, or C—CF₃.

Provided herein as Embodiment A97 is the compound or salt of any one of Embodiments A1-A91, wherein W₂ is C—C₂₋₃alkenyl or C—C₂₋₃alkynyl, and each of the alkenyl and alkynyl is unsubstituted or substituted with 1-3 substituents, and each substituent independently is halo, C₁₋₃haloalkyl, C₀₋₃alkyleneOH, or C₀₋₃alkyleneC₁₋₄alkoxy.

Provided herein as Embodiment A98 is the compound or salt of Embodiment A97, wherein W² is C—CH═CH₂, C—C(OH)═CH₂, C—CH═CH(OH), or C—CCH.

Provided herein as Embodiment A99 is the compound or salt of any one of Embodiments A1-A91, wherein W² is C—C₀₋₃alkyleneOH or C—C₀₋₃alkylene-C₁₋₄alkoxy.

Provided herein as Embodiment A100 is the compound or salt of Embodiment A99, wherein W² is C—OH, C—CH₂OH, C—OCH₃, or C—CH₂OCH₃.

Provided herein as Embodiment A101 is the compound or salt of any one of Embodiments A1-A91, wherein W² is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃.

Provided herein as Embodiment A102 is the compound or salt of any one of Embodiments A1-A81, wherein each of W₁ and W² independently is N, CH, or C—CH₃.

Provided herein as Embodiment A103 is the compound or salt of Embodiment A102, wherein W¹ is CH and W² is N, CH, or C—CH₃.

Provided herein as Embodiment A104 is the compound or salt of Embodiment A102, wherein W² is N and W₁ is N, CH, or C—CH₃.

Provided herein as Embodiment A105 is the compound or salt of Embodiment A102, wherein W¹ is CH and W² is N.

Provided herein as Embodiment A106 is the compound or salt of any one of Embodiments A1-A105, wherein X is N.

Provided herein as Embodiment A107 is the compound or salt of any one of Embodiments A1-A105, wherein X is C—R^{5a}.

Provided herein as Embodiment A108 is the compound or salt of Embodiment A107, wherein R^{5a} is H.

Provided herein as Embodiment A109 is the compound or salt of Embodiment A107, wherein R^{5a} is CN.

Provided herein as Embodiment A110 is the compound or salt of Embodiment A107, wherein R^{5a} is Br, Cl, or F.

Provided herein as Embodiment A111 is the compound or salt of Embodiment A107, wherein R^{5a} is C₁₋₃alkyl or C₁₋₃haloalkyl.

Provided herein as Embodiment A112 is the compound or salt of Embodiment A111, wherein R^{5a} is CH₃, CH₂CH₃, CF₃, CHF₂, or CH₂F.

Provided herein as Embodiment A113 is the compound or salt of Embodiment A112, wherein R^{5a} is CH₃.

Provided herein as Embodiment A114 is the compound or salt of Embodiment A107, wherein R^{5a} is C₂₋₆alkenyl or C₂₋₆alkynyl.

Provided herein as Embodiment A115 is the compound or salt of Embodiment A114, wherein R^{5a} is

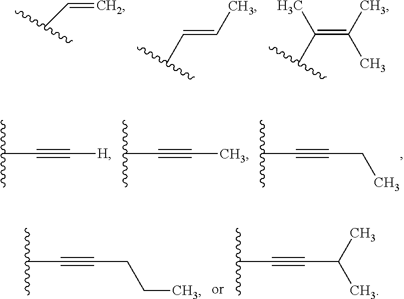

Provided herein as Embodiment A116 is the compound or salt of Embodiment A115, wherein R^{5a} is

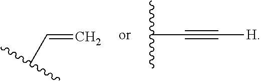

Provided herein as Embodiment A117 is the compound or salt of Embodiment A107, wherein R^{5a} is C₀₋₃alkyleneOH, C₀₋₃alkylene-C₁₋₃alkoxy, or C₃₋₅cycloalkyl.

Provided herein as Embodiment A118 is the compound or salt of Embodiment A117, wherein R^{5a} is OH, CH₂OH, OCH₃, CH₂OCH₃, or

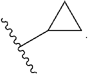 [SMILES: *C1CC1]

Provided herein as Embodiment A119 is the compound or salt of Embodiment A107, wherein R^{5a} is CN or CH₃.

Provided herein as Embodiment A120 is the compound or salt of any one of Embodiments A1-A105, wherein X is N, C—CH₃, or C—CN.

Provided herein as Embodiment A121 is the compound or salt of any one of Embodiments A1-A81, wherein W₁ is CH; W² is N; and X is N, C—CH₃, or C—CN.

Provided herein as Embodiment A122 is the compound or salt of any one of Embodiments A1-A121, wherein R^{5b} is Br, Cl, or F.

Provided herein as Embodiment A123 is the compound or salt of any one of Embodiments A1-A121, wherein R^{5b} is C₁₋₃haloalkyl.

Provided herein as Embodiment A124 is the compound or salt of Embodiment A123, wherein R^{5b} is CF₃, CF₂H, CFH₂, or CF₂CH₃.

Provided herein as Embodiment A125 is the compound or salt of Embodiment A124, wherein R^{5b} is CF₃ or CF₂H.

Provided herein as Embodiment A126 is the compound or salt of any one of Embodiments A1-A121, wherein R^{5b} is C₁₋₃alkoxy or C₁₋₃thioalkyl.

Provided herein as Embodiment A127 is the compound or salt of Embodiment A126, wherein R^{5b} is OCH₃ or SCH₃.

Provided herein as Embodiment A128 is the compound or salt of any one of Embodiments A1-A121, wherein R^{5b} is C₁₋₆alkyl, C₂₋₄alkenyl, or C₂₋₄alkynyl, and each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl.

Provided herein as Embodiment A129 is the compound or salt of Embodiment A128, wherein R^{5b} is CH₃, CH₂CH₃, CH₂CH₂CH₃, CH(CH₃)₂, CH═CH₂, CH═CHCH₃,

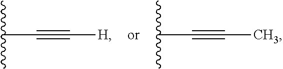

and each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl.

Provided herein as Embodiment A130 is the compound or salt of Embodiment A129, wherein each substituent independently is CH₃, CF₃, CF₂H, CFH₂, OH, OCH₃, OCF₃, CH₂OH, CH₂OCH₃, cyclopropyl, cyclobutyl, or phenyl.

Provided herein as Embodiment A131 is the compound or salt of Embodiment A129 or A130, wherein R^{5b} is CH₃, CH₂CH₃, CH₂CH₂CH₃, CH(CH₃)₂,

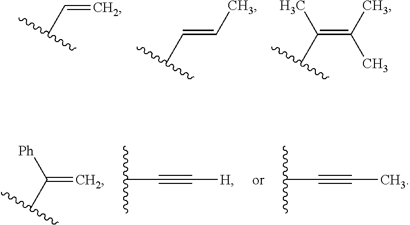

Provided herein as Embodiment A132 is the compound or salt of any one of Embodiments A1-A121, wherein R^{5b} is C₃₋₇cycloalkyl, C₅₋₇cycloalkenyl, heterocycloalkyl having 3-7 total ring atoms and 1-3 heteroatoms independently selected from N, O, and S, or heterocycloalkenyl having 5-7 total ring atoms and 1-3 heteroatoms independently selected from N, O, and S, wherein each of the foregoing is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl.

Provided herein as Embodiment A133 is the compound or salt of any one of Embodiments A1-A81, wherein

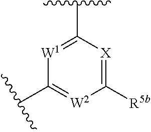

is

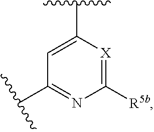

X is N, C—CH₃, or C—CN; and R^{b} is CF₃ or CF₂H.

Provided herein as Embodiment A134 is the compound or salt of any one of Embodiments A1-A81, wherein

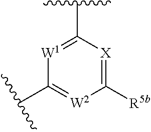

is

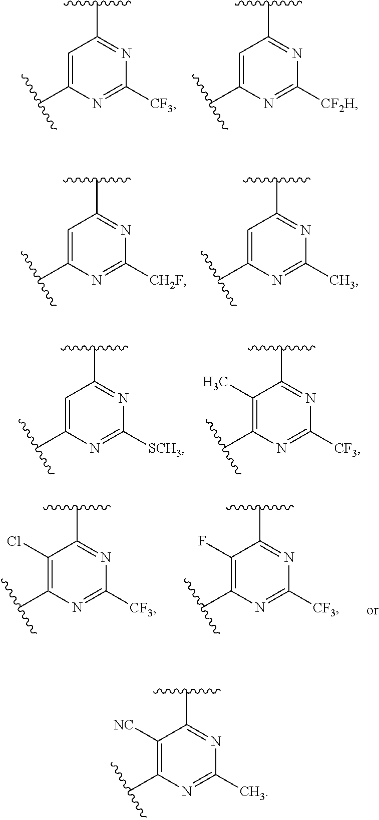

Provided herein as Embodiment A135 is the compound or salt of Embodiment A134, wherein

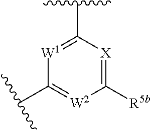

is

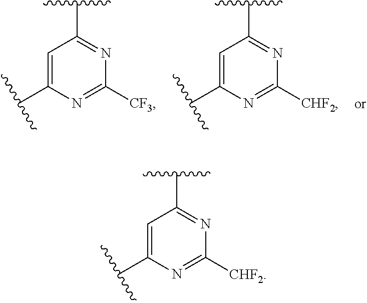 [SMILES: *c1cc(*)nc(C(F)(F)F)n1 ; *c1cc(*)nc(C(F)F)n1 ; *c1cc(*)nc(C(F)F)n1]

Provided herein as Embodiment A136 is the compound or salt of any one of Embodiments A1-A81, wherein

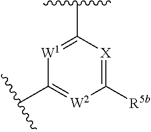

is

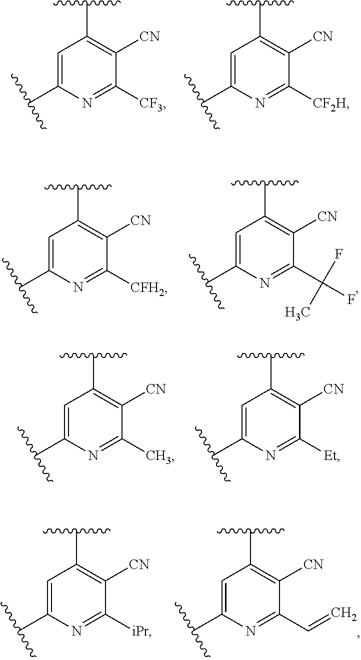

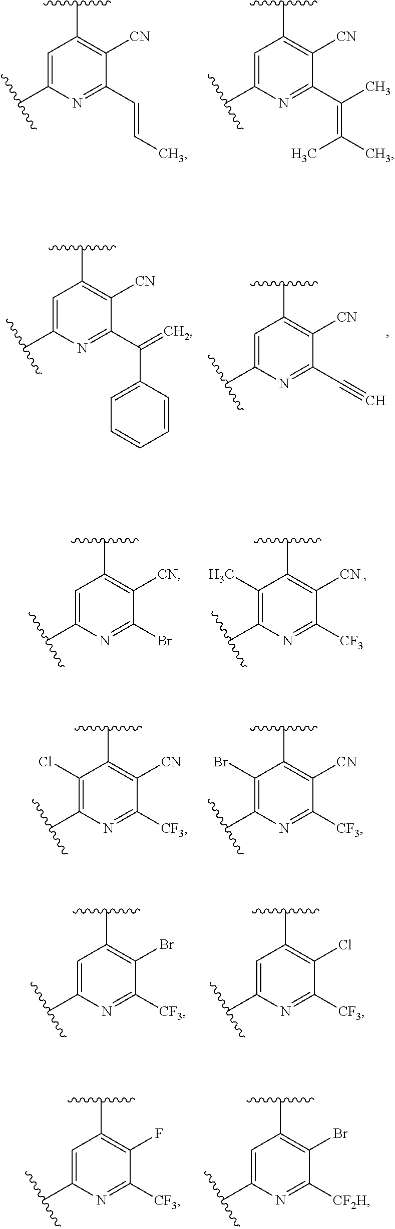

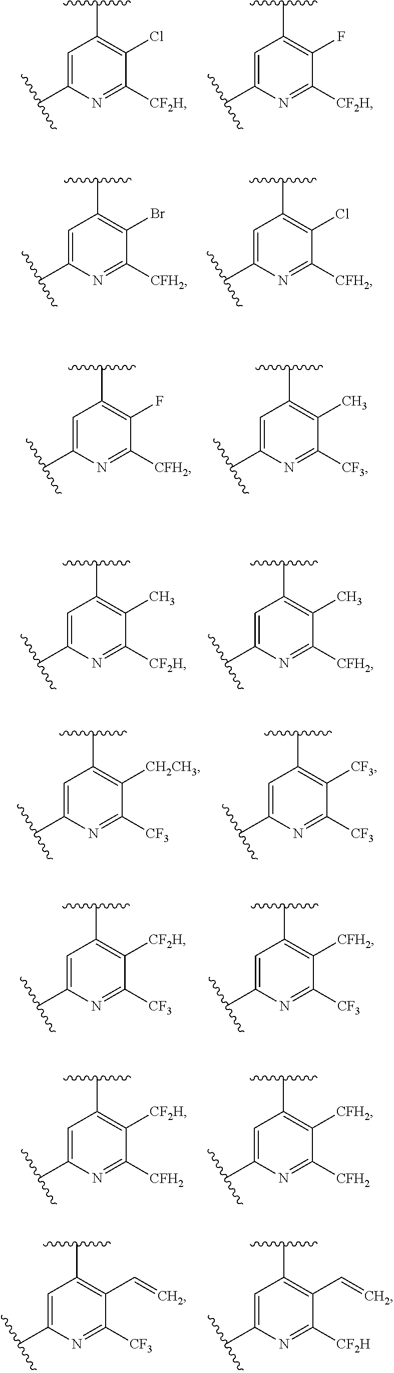

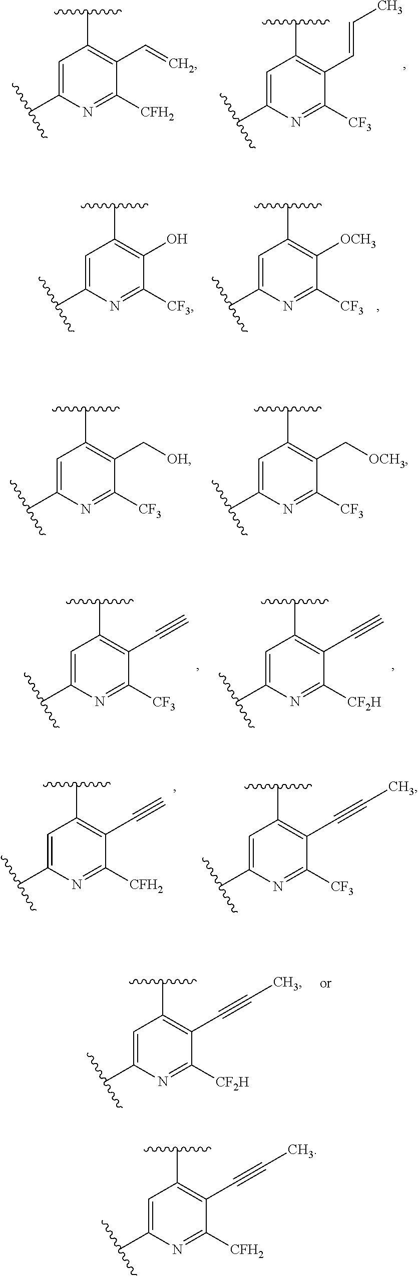

Provided herein as Embodiment A137 is the compound or salt of Embodiment A136, wherein

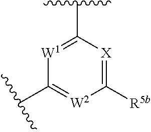

is

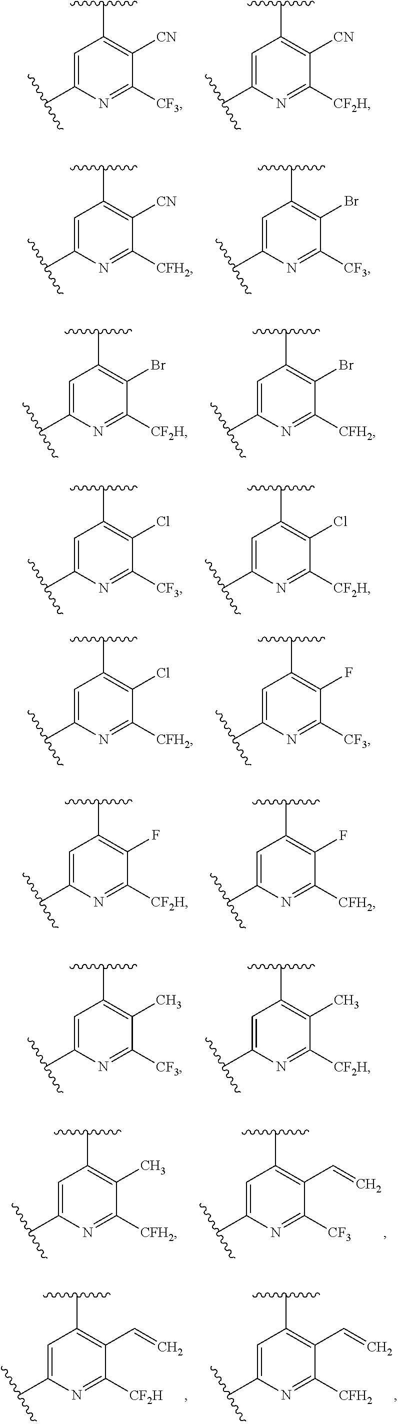

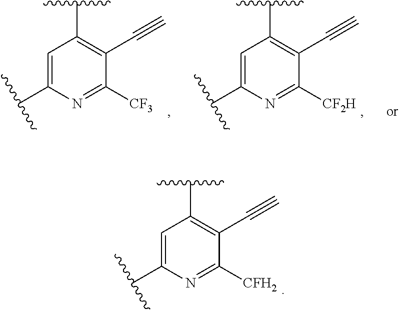

Provided herein as Embodiment A138 is the compound or salt of Embodiment A137, wherein

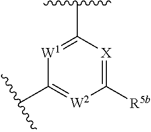

is

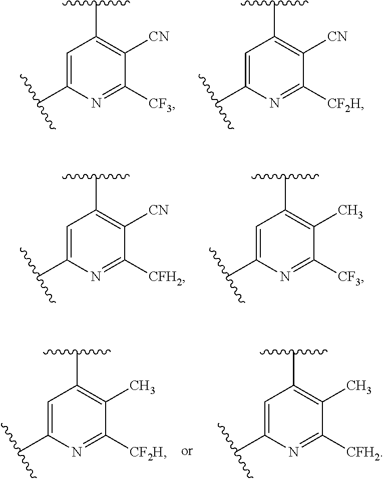

Provided herein as Embodiment A139 is the compound or salt of any one of Embodiments A1-A81, wherein

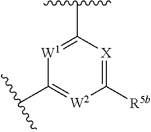

is

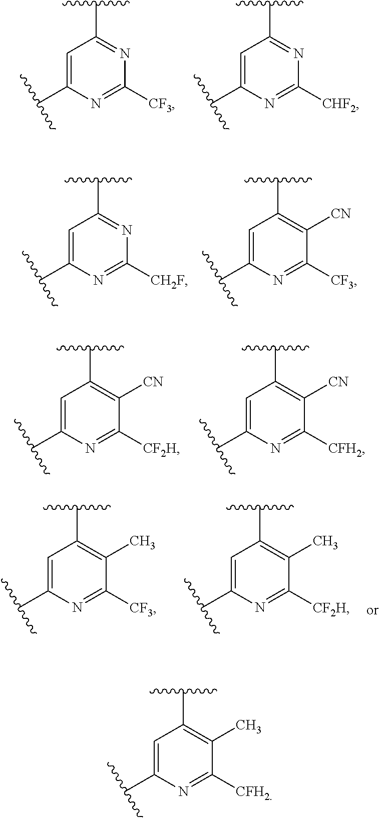

Provided herein as Embodiment A140 is the compound or salt of any one of Embodiments A1-A139, wherein q is 0.

Provided herein as Embodiment A141 is the compound or salt of any one of Embodiments A1-A139, wherein q is 1.

Provided herein as Embodiment A142 is the compound or salt of any one of Embodiments A1-A139, wherein q is 2.

Provided herein as Embodiment A143 is the compound or salt of any one of Embodiments A1-A142, wherein r is 0.

Provided herein as Embodiment A144 is the compound or salt of any one of Embodiments A1-A142, wherein r is 1.

Provided herein as Embodiment A145 is the compound or salt of any one of Embodiment A1-A139, wherein

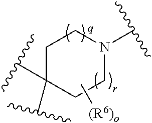

is

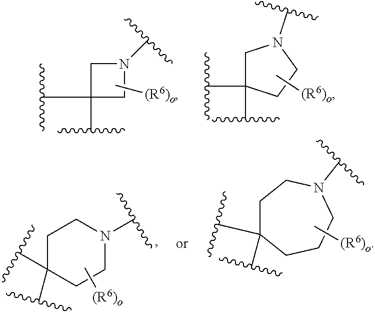

Provided herein as Embodiment A146 is the compound of any one of Embodiments A1-A145, wherein o is 0.

Provided herein as Embodiment A147 is the compound of any one of Embodiments A1-A145 wherein o is 1.

Provided herein as Embodiment A148 is the compound of any one of Embodiments A1-A145, wherein o is 2.

Provided herein as Embodiment A149 is the compound of any one of Embodiments A1-A145, wherein o is 3.

Provided herein as Embodiment A150 is the compound of any one of Embodiments A1-A145, wherein o is 4.

Provided herein as Embodiment A151 is the compound of any one of Embodiments A147-A150, wherein at least one R⁶ is Br, Cl, F, or CN.

Provided herein as Embodiment A152 is the compound or salt of Embodiment A151, wherein at least one R⁶ is F.

Provided herein as Embodiment A153 is the compound or salt of any one of Embodiments A147-A150, wherein at least one R⁶ is C₁₋₃alkyl or C₁₋₃haloalkyl.

Provided herein as Embodiment A154 is the compound or salt of Embodiment A153, wherein at least one R⁶ is CH₃, CH₂F, CHF₂, or CF₃.

Provided herein as Embodiment A155 is the compound or salt of any one of Embodiments A147-A150, wherein at least one R⁶ is C₀₋₃alkylene-OH, C₀₋₃alkylene-C₁₋₃alkoxy, deuterated C₀₋₃alkylene-C₁₋₃alkoxy, or C₁₋₄alkylene-N(R^{N1})₂, and each R^{N1} independently is H or CH₃.

Provided herein as Embodiment A156 is the compound or salt of Embodiment A155, wherein at least one R⁶ is OH, CH₂OH, OCH₃, OCD₃, CH₂OCH₃, or CH₂N(CH₃)₂.

Provided herein as Embodiment A157 is the compound or salt of any one of Embodiments A147-A150, wherein at least one R⁶ is oxo or ═CH₂.

Provided herein as Embodiment A158 is the compound or salt of any one of Embodiments A147-A150, wherein at least one R⁶ is C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S; wherein each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

Provided herein as Embodiment A159 is the compound or salt of Embodiment A158, wherein at least one R⁶ is cyclopropyl, cyclobutyl, oxetanyl, or tetrahydrofuranyl; wherein each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

Provided herein as Embodiment A160 is the compound or salt of Embodiment A159, wherein at least one R⁶ is cyclopropyl.

Provided herein as Embodiment A161 is the compound or salt of any one of Embodiments A147-A150, wherein at least one R⁶ is spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S, wherein each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

Provided herein as Embodiment A162 is the compound or salt of Embodiment A161, wherein at least one R⁶ is spiro-cyclopropyl, spiro-cyclobutyl, spiro-oxetanyl, or spiro-tetrahydrofuranyl, wherein each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₂alkoxy, or C₀₋₂alkyleneCN.

Provided herein as Embodiment A163 is the compound or salt of claim A162, wherein at least one R⁶ is spiro-cyclopropyl.

Provided herein as Embodiment A164 is the compound or salt of any one of Embodiments A148-A150, wherein two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; wherein each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

Provided herein as Embodiment A165 is the compound or salt of Embodiment A164, wherein two vicinal R⁶, together with the atoms to which they are attached, form fused-cyclopropyl, fused-cyclobutyl, or fused-cyclopentyl; wherein each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

Provided herein as Embodiment A166 is the compound or salt of any one of Embodiments A158, A159, A161, A162, A164, and A165, wherein each substituent independently is F, Cl, OH, OCH₃, OCH₂CH₃, or CN.

Provided herein as Embodiment A167 is the compound or salt of any one of Embodiments A148-A150, wherein two non-neighboring R⁶ join together to form a C₁₋₃alkylene bridge, a C₂₋₃alkenylene bridge, a C₁₋₃ether bridge, or a C₁₋₃thioether bridge.

Provided herein as Embodiment A168 is the compound or salt of Embodiment A167, wherein two non-neighboring R⁶ join together to form —CH₂—, —CH₂CH₂—, —CH₂CH₂CH₂—, —CH₂—CH═CH— or —CH₂OCH₂—.

Provided herein as Embodiment A169 is the compound or salt of Embodiment A168, wherein two non-neighboring R⁶ join together to form —CH₂—, —CH₂CH₂—, —CH₂CH₂CH₂—, or —CH₂OCH₂—.

Provided herein as Embodiment A170 is the compound or salt of any one of Embodiments A1-A145, wherein at least one R⁶ is Br, Cl, F, CN, CH₃, CH₂F, CHF₂, CF₃, OH, CH₂OH, OCH₃, OCD₃, CH₂OCH₃, CH₂N(CH₃)₂, oxo, ═CH₂, or cyclopropyl, or two vicinal R⁶, together with the atoms to which they are attached, form fused-cyclopropyl, fused-cyclobutyl, or fused-cyclopentyl, or two non-neighboring R⁶ join together to form —CH₂—, —CH₂CH₂—, —CH₂CH₂CH₂—; —CH₂—CH═CH— or —CH₂OCH₂—; wherein each of the foregoing cycloalkyl groups is unsubstituted or substituted with 1-4 substituents, and each substituent independently is F, Cl, OH, OCH₃, OCH₂CH₃, or CN.

Provided herein as Embodiment A171 is the compound or salt of any one of Embodiments A1-A145, wherein o is 0; or o is 1 and R⁶ is H, CH₃, CH₂OH, or two non-neighboring R⁶ join together to form —CH₂CH₂—.

Provided herein as Embodiment A172 is the compound or salt of any one of Embodiments A1-A145, wherein

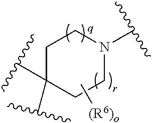

is

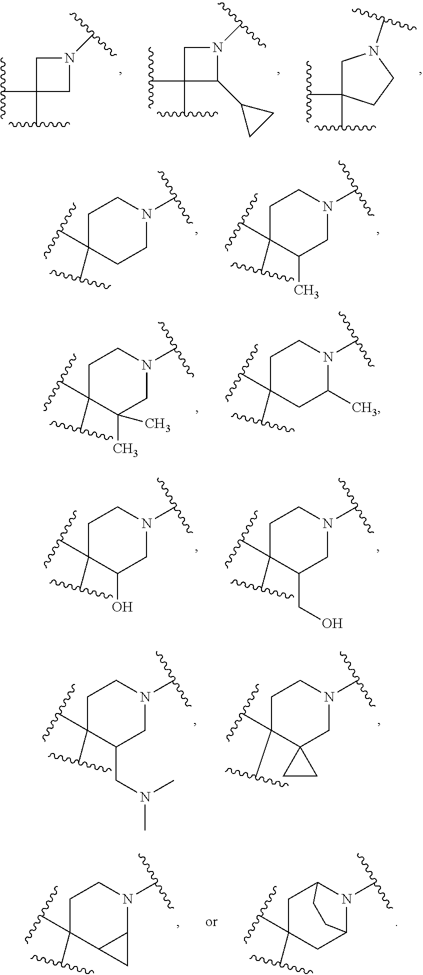

Provided herein as Embodiment A173 is the compound or salt of Embodiment A172, wherein

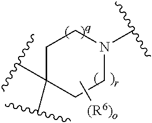

is

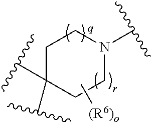

is

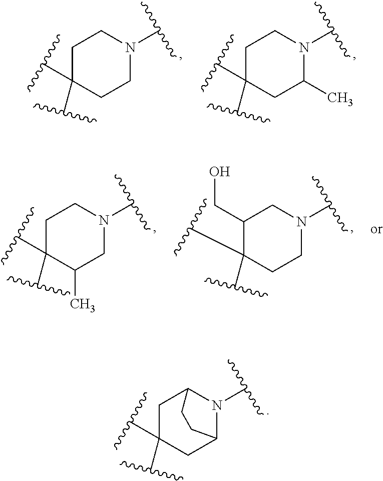

Provided herein as Embodiment A174 is the compound or salt of Embodiment A173, wherein

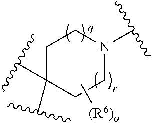

is

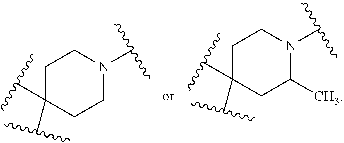 [SMILES: *N1CCC(*)(*)CC1 ; *N1CCC(*)(*)CC1C]

Provided herein as Embodiment A175 is the compound or salt of any one of Embodiments A1-A81, wherein the compound is a compound of Formula (IIA), Formula (IIB), or Formula (IIC):

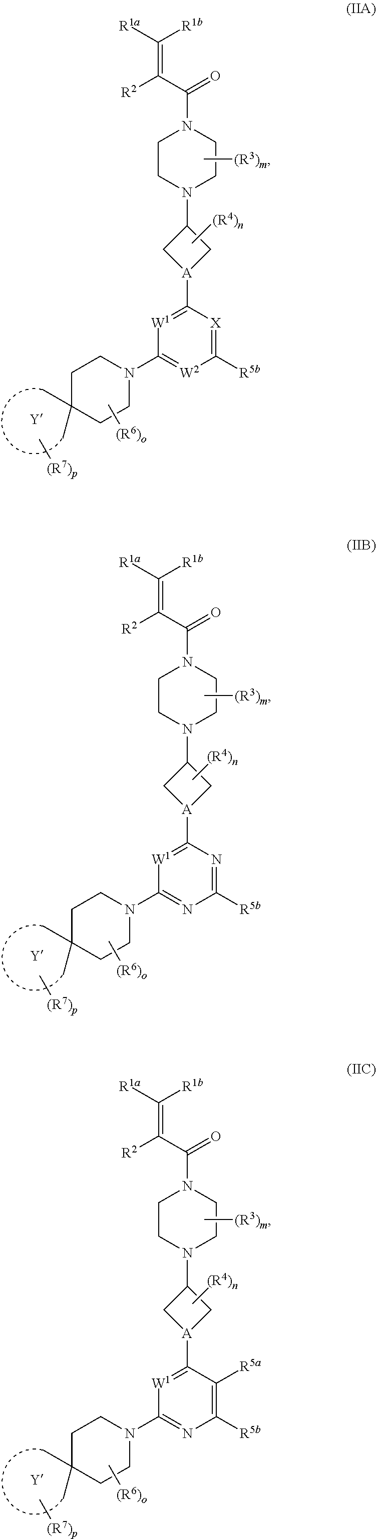

or a pharmaceutically acceptable salt of any of the foregoing.

Provided herein as Embodiment A176 is the compound or salt of any one of Embodiments A1-A175, wherein Y′ is C₃₋₇cycloalkyl or C₄₋₇cycloalkenyl.

Provided herein as Embodiment A177 is the compound or salt of Embodiment A176 wherein Y′ is cyclopropyl, cyclobutyl, cyclopentyl, cyclohexyl, cyclopentenyl, or cyclohexenyl.

Provided herein as Embodiment A178 is the compound or salt of Embodiment A177, wherein Y′ is cyclopropyl, cyclobutyl, cyclopentyl, or cyclopentenyl.

Provided herein as Embodiment A179 is the compound or salt of any one of Embodiments A1-A175, wherein Y′ is heterocycloalkyl having 3 to 7 total ring atoms and 1-3 heteroatoms independently selected from N, O, and S.

Provided herein as Embodiment A180 is the compound or salt of Embodiment A179, wherein Y′ is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, dioxolanyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothiopyranyl, dithianyl, morpholinyl, or thiomorpholinyl.

Provided herein as Embodiment A181 is the compound or salt of Embodiment A180, wherein Y′ is oxetanyl, pyrrolidinyl, tetrahydrofuranyl, dioxolanyl, oxazolidinyl, isoxazolidinyl, tetrahydropyranyl, or morpholinyl.

Provided herein as Embodiment A182 is the compound or salt of Embodiment A181, wherein Y′ is pyrrolidinyl, tetrahydrofuranyl, or tetrahydropyranyl.

Provided herein as Embodiment A183 is the compound or salt of any one of Embodiments A1-A175, wherein Y′ is heterocycloalkenyl having 5 to 7 total ring atoms and 1-3 heteroatoms independently selected from N, O, and S.

Provided herein as Embodiment A184 is the compound or salt of Embodiment A183, wherein Y′ is dihydropyrrolyl, dihydrofuranyl, dihydrothiopheneyl, dihydroisoxazolyl, tetrahydropyridinyl, dihydropyranyl, or dihydrothipyranyl.

Provided herein as Embodiment A185 is the compound or salt of Embodiment A184, wherein Y′ is dihydropyrrolyl, dihydrofuranyl or dihydropyranyl.

Provided herein as Embodiment A186 is the compound or salt of any one of Embodiments A1-A175, wherein Y′ is cyclopropyl, cyclobutyl, cyclopentyl, cyclohexyl, cyclopentenyl, cyclohexenyl, aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, dioxolanyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothiopyranyl, dithianyl, morpholinyl, thiomorpholinyl, dihydropyrrolyl, dihydrofuranyl, dihydrothiophencyl, dihydroisoxazolyl, tetrahydropyridinyl, dihydropyranyl, or dihydrothipyranyl.

Provided herein as Embodiment A187 is the compound or salt of Embodiment A186, wherein Y′ is cyclopropyl, cyclobutyl, cyclopentyl, cyclopentenyl, oxetanyl, pyrrolidinyl, tetrahydrofuranyl, dioxolanyl, oxazolidinyl, isoxazolidinyl, tetrahydropyranyl, dihydropyrrolyl, dihydrofuranyl or dihydropyranyl.

Provided herein as Embodiment A188 is the compound or salt of any one of Embodiments A1-A187, wherein p is 0.

Provided herein as Embodiment A189 is the compound or salt of any one of Embodiments A1-A187, wherein p is 1.

Provided herein as Embodiment A190 is the compound or salt of any one of Embodiments A1-A187, wherein p is 2.

Provided herein as Embodiment A191 is the compound or salt of any one of Embodiments A1-A187, wherein p is 3.

Provided herein as Embodiment A192 is the The compound or salt of any one of Embodiments A1-A187, wherein p is 4.

Provided herein as Embodiment A193 is the compound or salt of any one of Embodiments A1-A187, wherein p is 5.

Provided herein as Embodiment A194 is the compound or salt of any one of Embodiment A189-A193, wherein at least one R⁷ is F, Cl, Br, CN, or CH₂CN.

Provided herein as Embodiment A195 is the compound or salt of Embodiment A194, wherein at least one R⁷ is F or CH₂CN.

Provided herein as Embodiment A196 is the compound or salt of any one of Embodiments A189-A193, wherein at least one R⁷ is C₁₋₃alkyl, C₁₋₃haloalkyl, or C₂₋₃alkenyl.

Provided herein as Embodiment A197 is the compound or salt of Embodiment A196, wherein at least one R⁷ is CH₃, CH₂CH₃, CH₂F, CHF₂, or CF₃.

Provided herein as Embodiment A198 is the compound or salt of Embodiment A197, wherein at least one R⁷ is CH₃, CH₂F, or CHF₂.

Provided herein as Embodiment A199 is the compound or salt of any one of Embodiments A189-A193, wherein at least one R⁷ is C₀₋₃alkyleneOH or C₀₋₃alkylene-C₁₋₃alkoxy.

Provided herein as Embodiment A200 is the compound or salt of Embodiment A199, wherein at least one R⁷ is OH, OCH₃, OCH₂CH₃, CH₂OH, CH₂OCH₃, or CH₂CH₂OCH₃.

Provided herein as Embodiment A201 is the compound or salt of Embodiment A200, wherein at least one R⁷ is OH or CH₂OCH₃.

Provided herein as Embodiment A202 is the compound or salt of any one of Embodiments A189-A193, wherein at least one R⁷ is C(═O)OCH₃.

Provided herein as Embodiment A203 is the compound or salt of any one of Embodiments A189-A193, wherein at least one R⁷ is oxo or ═CH₂.

Provided herein as Embodiment A204 is the compound or salt of any one of Embodiments A189-A193, wherein at least one R⁷ is C₀₋₄alkylene-C₃₋₇cycloalkyl; C₀₋₄alkylene-heterocycloalkyl having 3-7 total ring atoms and 1-3 heteroatoms independently selected from N, O, and S; C₀₋₄alkylene-C₆₋₁₀aryl; or C₀₋₄alkylene-heteroaryl having 4-7 total ring atoms and 1-3 heteroatoms independently selected from N, O, and S; wherein each of the foregoing is unsubstituted or substituted with 1-3 substituents, and each substituent independently is halo, CN, oxo, or C₁₋₃alkyl.

Provided herein as Embodiment A205 is the compound or salt of Embodiment A204, wherein at least one R⁷ is C₀₋₃alkylene-cyclopropyl, C₀₋₄alkylene-cyclobutyl, C₀₋₄alkylene-cyclopentyl, C₀₋₄alkylene-oxetanyl, C₀₋₄alkylene-pyrrolidinyl, C₀₋₄alkylene-phenyl, or C₀₋₄alkylene-oxadiazolyl.

Provided herein as Embodiment A206 is the compound or salt of Embodiment A205, wherein at least one R⁷ is cyclopropyl.

Provided herein as Embodiment A207 is the compound or salt of any one of Embodiments A189-A193, wherein at least one R⁷ is spiro-C₃₋₇cycloalkyl or a spiro-heterocycloalkyl ring having 4-7 total ring atoms and 1 or 2 heteroatoms independently selected from N, O and S; wherein each of the foregoing is unsubstituted or substituted with 1-3 substituents, and each substituent independently is halo, CN, oxo, or C₁₋₃alkyl.

Provided herein as Embodiment A208 is the compound or salt of Embodiment A207, wherein at least one R⁷ is spiro-cyclopropyl, spiro-cyclobutyl, spiro-oxetanyl, spiro-tetrahydrofuranyl, or spiro-tetrahydropyranyl.

Provided herein as Embodiment A209 is the compound or salt of any one of Embodiments A190-A193, wherein two vicinal R⁷, together with the atoms to which they are attached, form fused-Ca-cycloalkyl or fused-heterocycloalkyl having 3-7 total ring atoms and 1-3 heteroatoms independently selected from N, O, and S; wherein each of the foregoing is unsubstituted or substituted with 1-3 substituents, and each substituent independently is halo, CN, oxo, or C₁₋₃alkyl.

Provided herein as Embodiment A210 is the compound or salt of Embodiment A209, wherein two vicinal R⁷, together with the atoms to which they are attached, form fused-cyclopropyl, fused-cyclobutyl, fused-oxetanyl, fused-tetrahydrofuranyl, or fused-tetrahydropyranyl.

Provided herein as Embodiment A211 is the compound or salt of Embodiment A210, wherein two vicinal R⁷, together with the atoms to which they are attached, form fused-tetrahydrofuranyl.

Provided herein as Embodiment A212 is the compound or salt of any one of Embodiments A190-A193, wherein two vicinal R⁷, together with the atoms to which they are attached, form fused-C₆₋₁₀aryl or fused-heteroaryl having 5-7 total ring atoms and 1-3 heteroatoms independently selected from N, O, and S; wherein each of the foregoing is unsubstituted or substituted with 1-3 substituents, and each substituent independently is halo, CN, oxo, or C₁₋₃alkyl.

Provided herein as Embodiment A213 is the compound or salt of Embodiment A212, wherein two vicinal R⁷, together with the atoms to which they are attached, form fused-phenyl, fused-thiophenyl, fused-pyrazolyl, fused-thiazolyl, fused-pyridyl, fused-pyridazinyl, or fused-pyrazinyl; wherein each of the foregoing is unsubstituted or substituted with 1-3 substituents, and each substituent independently is halo, CN, oxo, or C₁₋₃alkyl.

Provided herein as Embodiment A214 is the compound or salt of Embodiment A213, wherein two vicinal R⁷, together with the atoms to which they are attached, form fused-pyrazolyl, fused-pyridyl, fused-pyridazinyl, or fused-pyrimidinyl; wherein each of the foregoing is unsubstituted or substituted with 1-3 substituents, and each substituent independently is halo, CN, oxo, or C₁₋₃alkyl.

Provided herein as Embodiment A215 is the compound or salt of any one of Embodiments A204-A214, wherein each substituent independently is F, Cl, Br, CN, oxo, or CH₃.

Provided herein as Embodiment A216 is the compound or salt of any one of Embodiments A1-A187, wherein each R⁷ independently is F, Cl, Br, CN, CH₂CN, CH₃, CH₂CH₃, CH₂F, CHF₂, CF₃, OH, OCH₃, OCH₂CH₃, CH₂OH, CH₂OCH₃, CH₂CH₂OCH₃, C(═O)OCH₃, oxo, ═CH₂, cyclopropyl, spiro-cyclopropyl, spiro-cyclobutyl, spiro-oxetanyl, spiro-tetrahydrofuranyl, spiro-tetrahydropyranyl; or two vicinal R⁷, together with the atoms to which they are attached, form fused-cyclopropyl, fused-cyclobutyl, fused-oxetanyl, fused-tetrahydrofuranyl, fused-phenyl, fused-thiophenyl, fused-pyrazolyl, fused-thiazolyl, fused-pyridyl, fused-pyridazinyl, fused-pyrazinyl, fused-pyrimidinyl; wherein each of the pyrazolyl, pyridyl, pyridazinyl, and pyrimidinyl is unsubstituted or substituted with CH₃.

Provided herein as Embodiment A217 is the compound or salt of any one of Embodiments A1-A175, wherein

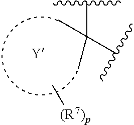

is

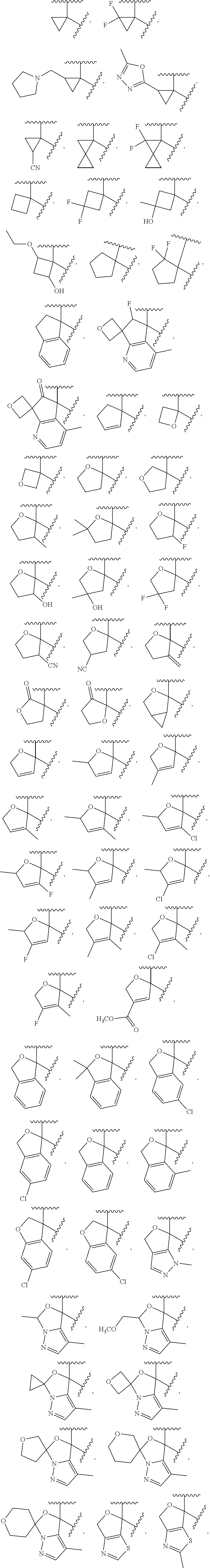

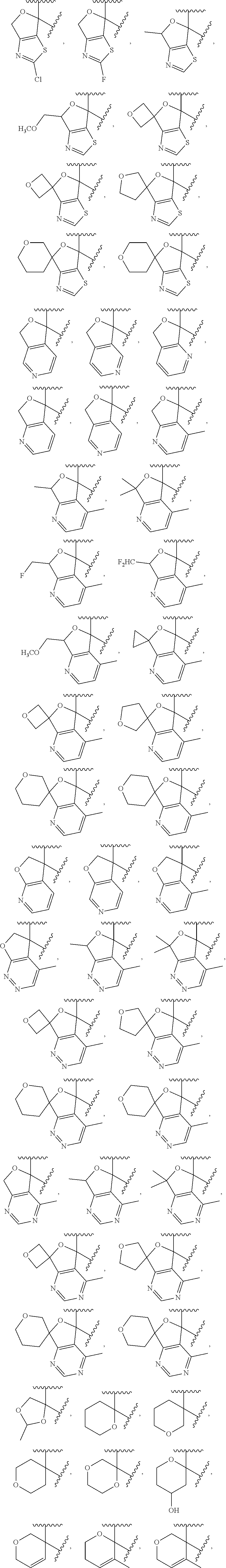

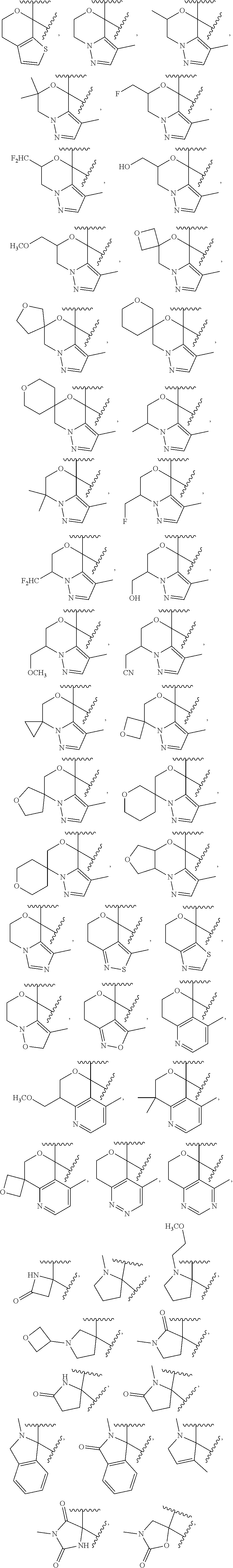

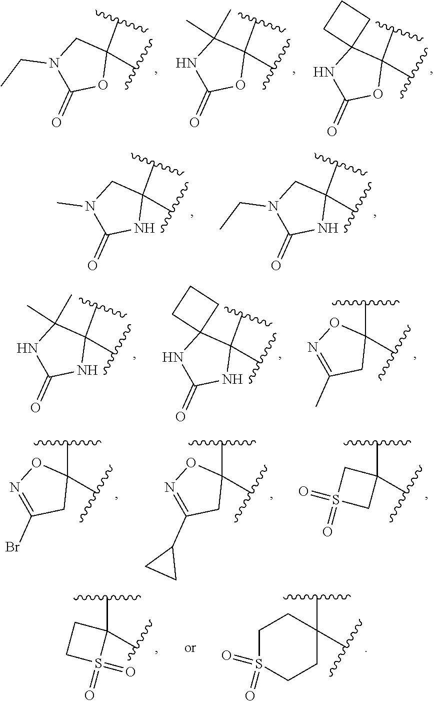

Provided herein as Embodiment A218 is the compound or salt of any one of Embodiments A1-A175, wherein

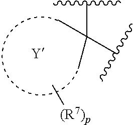

is

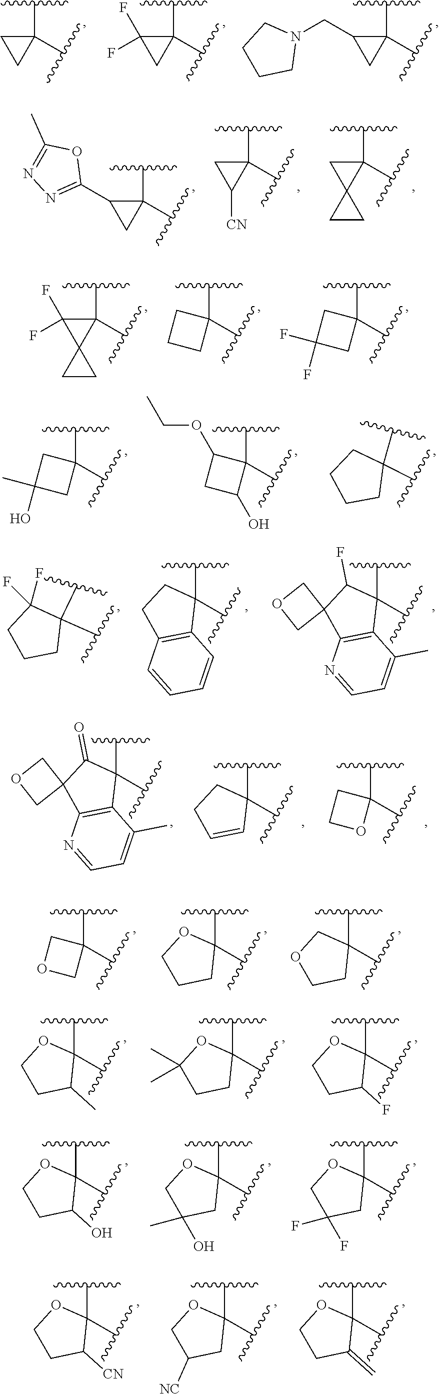

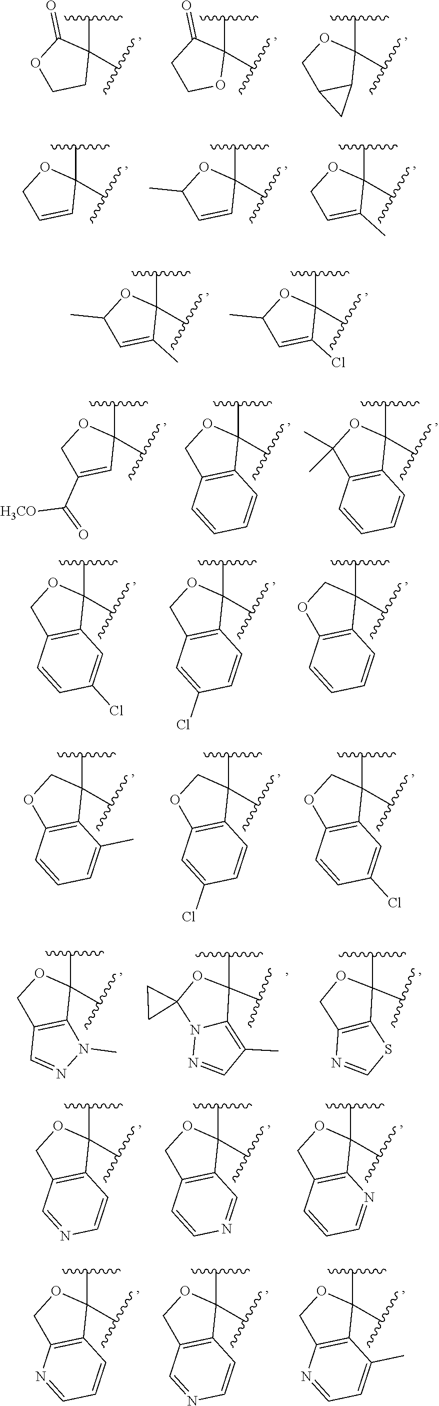

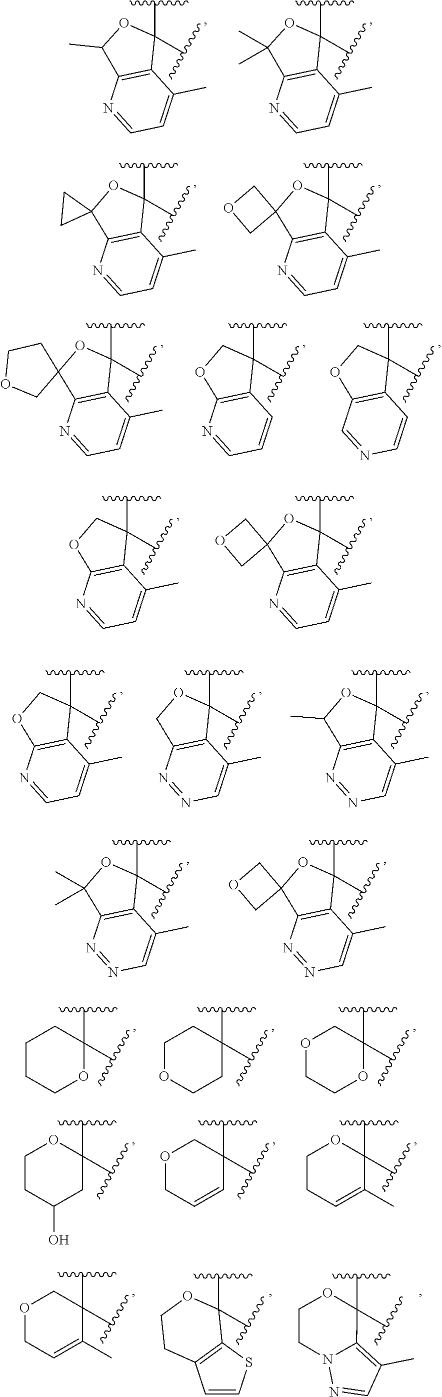

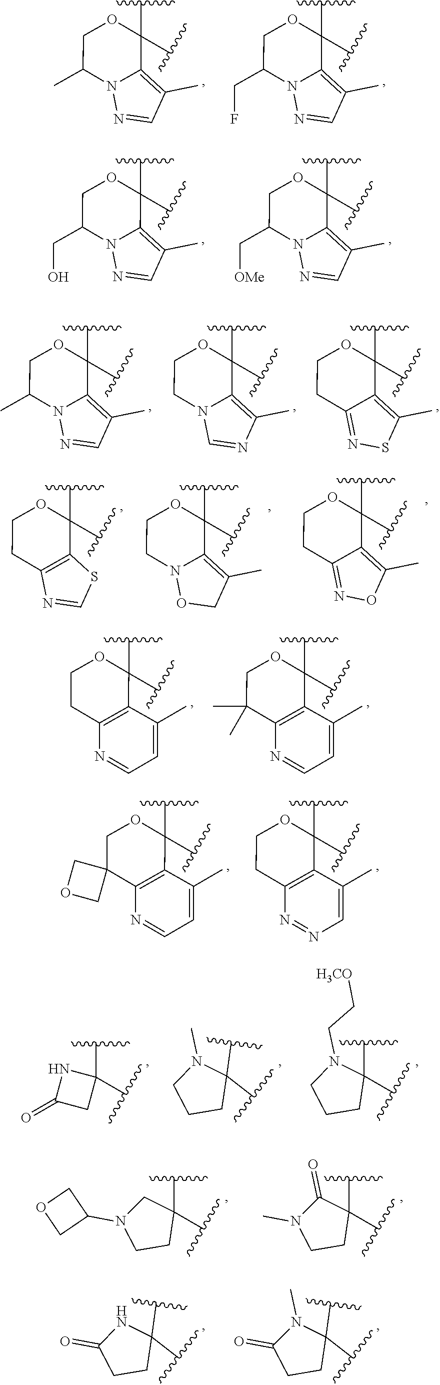

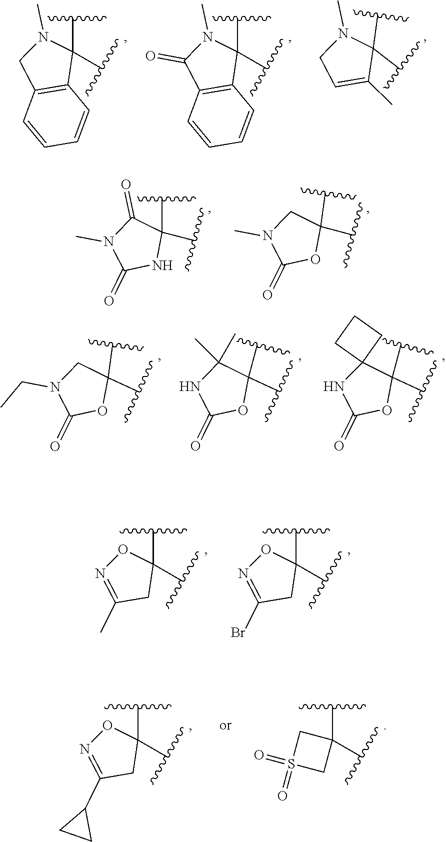

Provided herein as Embodiment A219 is the compound or salt of Embodiment A218, wherein

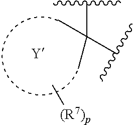

is

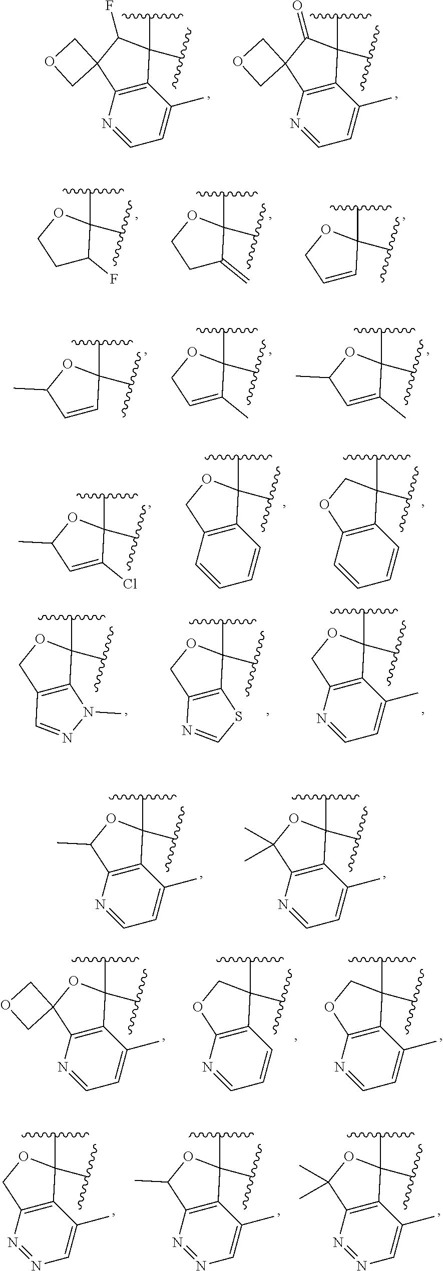

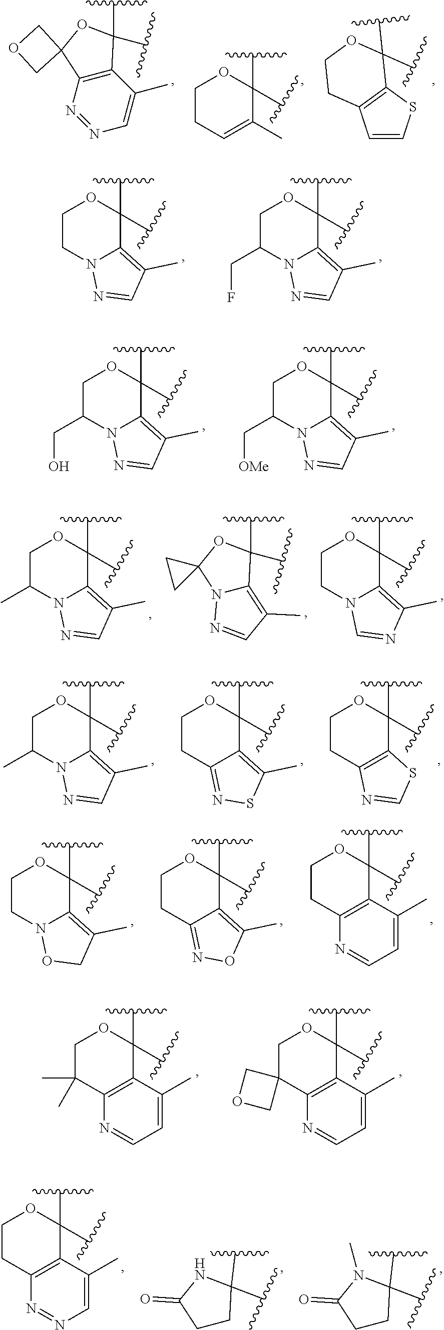

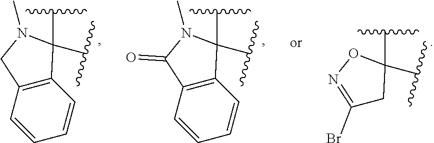

Provided herein as Embodiment A220 is the compound or salt of Embodiment A1, wherein:

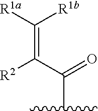

is

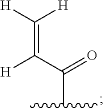 [SMILES: *C(=O)C=C]

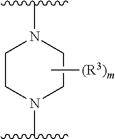

is

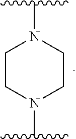 [SMILES: *N1CCN(*)CC1]

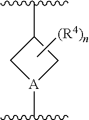

is

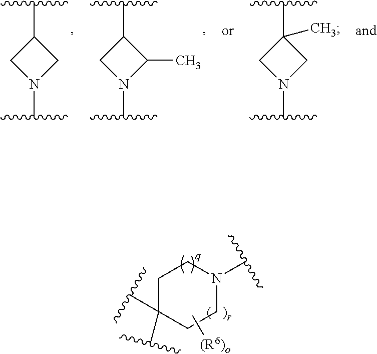

is

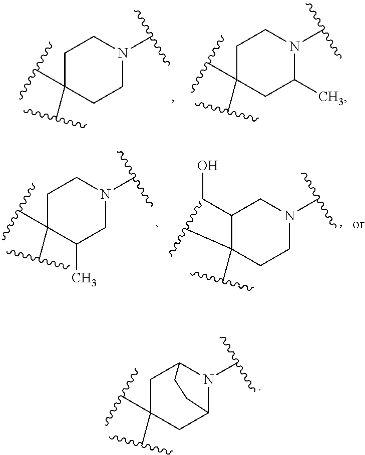

Provided herein as Embodiment A221 is the compound of Embodiment A220, wherein

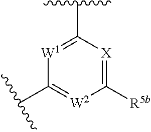

is

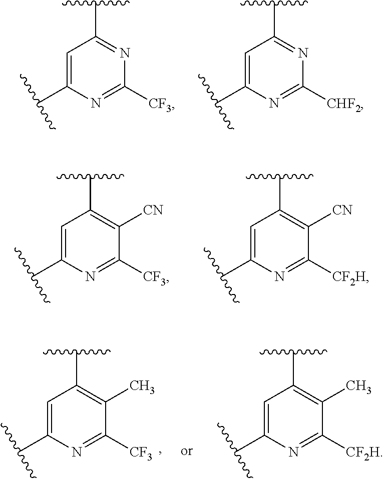

Provided herein as Embodiment A222 is the compound or salt of Embodiment A220 or A221, wherein

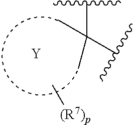

is

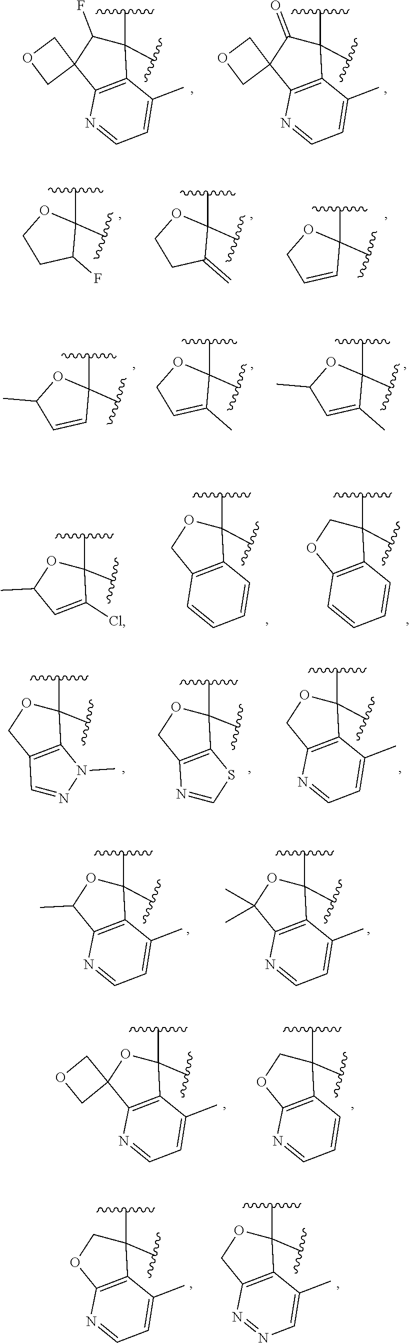

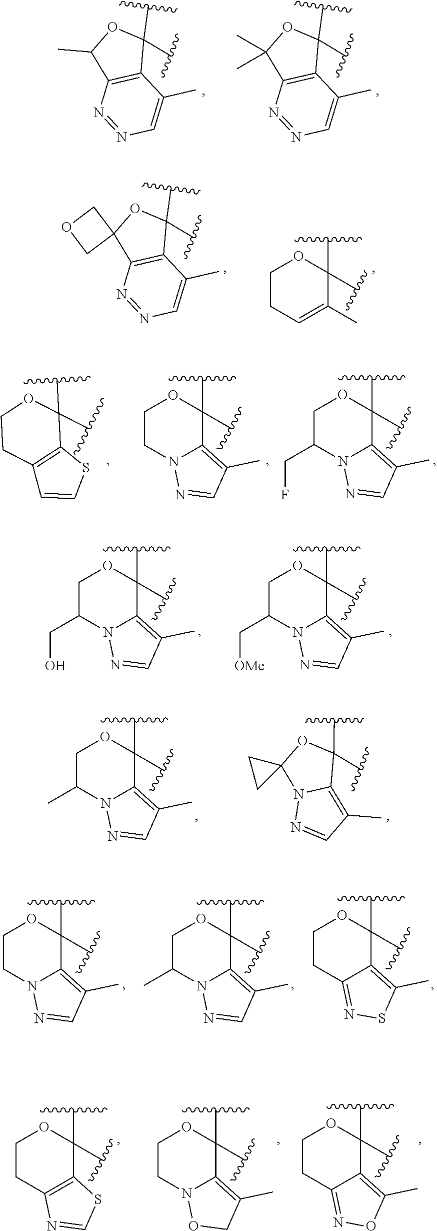

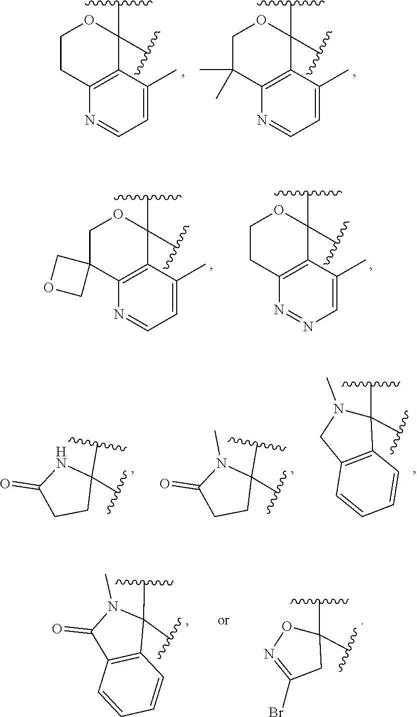

Provided herein as Embodiment A223 is the compound or salt of any one of Embodiments A1-A81, wherein q is 1; r is 1; W¹ is CH, C-halo, C—CN, C—C₁₋₃alkyl, C—C₁₋₃haloalkyl, C—C₀₋₃alkyleneOH, or C—C₀₋₃alkylene-C₁₋₄alkoxy; W² is N; and X is N or C—R^{5a}.

Provided herein as Embodiment A224 is the compound of Embodiment A1, wherein the compound is a compound listed in Table A, or a pharmaceutically acceptable salt thereof.

Provided herein as Embodiment A225 is the compound of Embodiment A224, wherein the compound is a compound listed in Table B, or a pharmaceutically acceptable salt thereof.

Provided herein as Embodiment A226 is the compound of Embodiment A1, wherein the compound is a compound listed in Table C, or a pharmaceutically acceptable salt thereof.

Provided herein as Embodiment A227 is the compound of Embodiment A226, wherein the compound is a compound listed in Table D, or a pharmaceutically acceptable salt thereof.

Provided herein as Embodiment A228 is a pharmaceutical composition comprising the compound or salt of any one of Embodiments A1-A227 and a pharmaceutically acceptable excipient.

Provided herein as Embodiment A229 is a method of treating cancer in a subject in need of treatment, the method comprising administering to the subject a therapeutically effective amount of the compound or salt of any one of Embodiments A1-227, or the composition of Embodiment A228.

Provided herein as Embodiment A230 is the method of Embodiment A229, wherein one or more cancer cells express KRAS G12C mutant protein.

Provided herein as Embodiment A231 is the method of Embodiment A229 or A230, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Provided herein as Embodiment A232 is the method of Embodiment A231, wherein the cancer is non-small cell lung cancer, colorectal cancer, pancreatic cancer, or solid tumor.

Provided herein as Embodiment A233 is the method according to any one of Embodiments A229-A232, wherein the subject has a cancer that was determined to have one or more cells expressing the KRAS G12C mutant protein prior to administration of the compound, salt, or pharmaceutical composition.

Provided herein as Embodiment A234 is the method according to any one of Embodiments A229-A233, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor. FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

Provided herein as Embodiment A235 is the compound or salt of any one of Embodiments A1-A227, or the composition of Embodiment A229 for use as a medicament.

Provided herein as Embodiment A236 is the compound or salt of any one of Embodiments A1-A227, or the composition of Embodiment A228 for use in treating cancer.

Provided herein as Embodiment A237 is the compound or salt of any one of Embodiments A1-A227 or the pharmaceutical composition of Embodiment A228 for use in treating cancer, wherein one or more cancer cells express KRAS G12C mutant protein.

Provided herein as Embodiment A238 is the compound or salt of Embodiment A236 or A237, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment A239 is the compound or salt of Embodiment A238, wherein the cancer is non-small cell lung cancer colorectal cancer, pancreatic cancer, or solid tumor.

Provided herein as Embodiment A240 is a use of a compound or salt of any one of Embodiments A1-A227 or the pharmaceutical composition of Embodiment A228 for the manufacture of a medicament for the treatment of cancer.

Provided herein as Embodiment A241 is a use of a compound or salt of any one of Embodiments A1-A227 or the pharmaceutical composition of Embodiment A228 in the preparation of a medicament for treating cancer, wherein one or more cancer cells express KRAS^{G12C} mutant protein.

Provided herein as Embodiment A242 is the use of Embodiment A240 or A241, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Provided herein as Embodiment A243 is the use of Embodiment A242, wherein the cancer is non-small cell lung cancer, colorectal cancer, pancreatic cancer, or solid tumor.

Provided herein as Embodiment A244 is a compound of Formula (B):

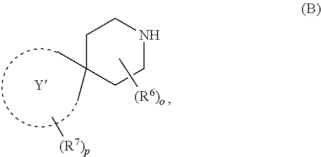

or a nitrogen-protected analog, or a pharmaceutically acceptable salt of any of the foregoing, wherein:
o is 0, 1, 2, 3, or 4;
p is 0, 1, 2, 3, 4, or 5;
each R⁶ s halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring Rejoin together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

Provided herein as Embodiment A245 is a method of preparing the compound or salt of any one of Embodiments A1-A227, comprising admixing a compound of Formula (B):

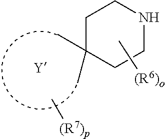

(B) with a compound:

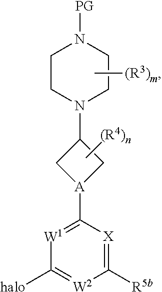

under carbon-nitrogen bond-forming conditions to form:

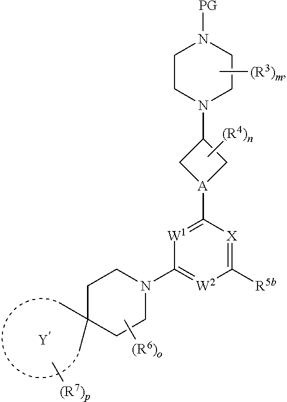

wherein PG is a protecting group.

Provided herein as Embodiment A246 is a compound listed in Table E, Table F. Table G, a compound selected from selected from the group consisting of 3-001.1, 3-001.2, 3-002.1, 3-002.2, 3-003.1, 3-003.2, 3-020.1, 3-020.2, 3-021.1, 3-021.2, 3-046.1, 3.046.2, 3-050.1, 3-050.2, 3-051.1, 3-051.2, 3-051.3, 3-051.4, 3-051.5, 3-052.1, 3-052.2, 3-052.3, 3-052.4, 3-052.5, 3-053.1, 3-053.2, 3-053.3, 3-053.4, 3-053.5, 3-054-1, 3-054-2, 3-055.1, 3-055.2, 3-055.3, 3-055.4, 3-055.5, 3-057.1, 3-057.2, 3-057.3, 3-058, 1, 3-058.2-1, 3-058.2.2, 3-058.3, 3-059.1, 3-060.1, 3-060.2, 3-060.3, 3-060.4, 3-060.5, 3-060.6, 3-060.7, 3-060.8, 3-060.9, 3-061.1, 3-061.2, 3-061.3, 3-061.4, 3-061.5, 3-062.1-1, 3-062.1-2, 3-062.1-1, 3-062.2, 3-063.1, 3-063.2, 3-065.1, 3-065.2, 3-065.3, 3-065.4, 3-065.5, 3-067-1, 3-067-2, 3-069.1, 3-069.2, 3-073.1, 3-073.2, 3-073.3, 3-073.4, 3-073.5, 3-074.1, 3-074.2, 3-074.3, 3-074.4, 3-075.1, 3-075.2, 3-075.3, 3-076.1, and 3-076.2, a protected analog of any of the foregoing, or a pharmaceutically acceptable salt of any of the foregoing.

Provided herein as Embodiment A247 is a method of preparing the compound or salt of any one of Embodiments A1-A243, comprising converting a compound of Embodiment A247, a protected analog of any of the foregoing, or a pharmaceutically acceptable salt of any of the foregoing, into a compound of any one of Embodiments A1-A243.

### OTHER EMBODIMENTS

Provided herein as Embodiment 1 is a compound of Formula (1):

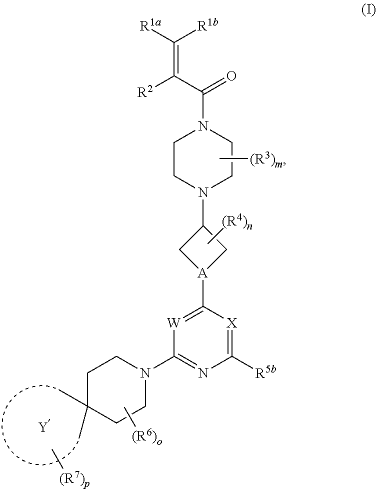

or a pharmaceutically acceptable salt thereof,
wherein:
m is 0, 1, 2, 3, or 4;
n is 0, 1, or 2;
o is 0, 1, 2, 3, or 4;
p is 0, 1, 2, 3, 4, or 5;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
W is CH, C-halo, C—CN, C—C₁₋₃alkyl, C—C₁₋₃haloalkyl, C—C₀₋₃alkyleneOH, or C—C₀₋₃alkylene-C₁₋₄alkoxy;
X is N or C—R^{5a};
Y′ is cycloalkyl having 3 to 7 total ring atoms, cycloalkenyl having 4 to 7 total ring atoms, heterocycloalkyl having 3 to 7 total ring atoms and 1, 2, or 3 heteroatoms selected from N, O, and S, or heterocycloalkenyl having 5 to 7 total ring atoms and 1, 2, or 3 heteroatoms selected from N, O, and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R² together with the carbon atoms to which they are attached from a

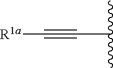

group;
each R³ independently is C₁₋₃alkyl, C₁₋₃haloalkyl,

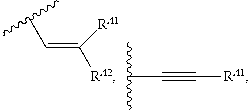

C₀₋₃alkyleneCN, C₀₋₃alkyleneOH, or C₀₋₃alkylene-C₁₋₃alkoxy; two geminal R³ form an oxo group; two geminal R³ together with the atom to which they are attached form a spiro-cycloalkyl ring having 3-7 total ring atoms or a spiro-heterocycloalkyl ring having 3-7 total ring atoms and 1 or 2 heteroatoms selected from N, O and S; or two adjacent R³ together with the atoms to which they are attached form a fused cycloalkyl ring having 3-7 total ring atoms;
each R⁴ independently is C₁₋₃alkyl, C₁₋₃haloalkyl, C₀₋₃alkyleneCN, C₁₋₃alkyleneOH, or C₁₋₃alkylene-C₁₋₃alkoxy; two geminal R³ form an oxo group; or two geminal R⁴ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S;
R^{5a} is H, CN, halo, C₁₋₃alkyl, C₁₋₃haloalkyl, C₂₋₆alkenyl, C₂₋₆alkynyl, C₀₋₃alkyleneOH, C₀₋₃alkylene-C₁₋₃alkoxy, or cycloalkyl having 3-5 total ring atoms;
R^{5b} is C₁₋₄alkyl, C₂₋₃alkenyl, C₂₋₃alkynyl, C₁₋₃haloalkyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the alkyl, alkenyl, and alkynyl groups is independently unsubstituted or substituted with 1 or more substituents, or R^{5a} and R^{5b} together with the atoms to which they are attached form a cycloalkyl ring having 3-7 total ring atoms;
each R⁶ independently is halo, CN, C₁₋₃alkyl, C₁₋₃haloalkyl, C₀₋₃alkyleneOH, C₀₋₃alkylene-C₁₋₃alkoxy, deuterated C₀₋₃alkylene-C₁₋₃alkoxy, or C₁₋₄alkylene-N(R^{N1})₂; two geminal R⁶ form oxo; two geminal R⁶ together with the atom to which they are attached from a spiro-cycloalkyl ring having 3-7 total ring atoms or a spiro-heterocycloalkyl ring having 4-7 total ring atoms and 1 or 2 heteroatoms selected from N, O and S; or two adjacent R⁶ together with the atoms to which they are attached form a fused cycloalkyl ring having 3-7 total ring atoms; wherein the cycloalkyl or heterocycloalkyl ring of any of the foregoing is unsubstituted or substituted with 1 or more substituents; or two non-adjacent Rejoin together to form a C₁₋₃alkylene bridge or a C₁₋₃ether bridge;
each R⁷ independently is halo, CN, C₁₋₃alkyl, C₁₋₃haloalkyl, C₂₋₃alkenyl, C₀₋₂alkyleneOH, C₀₋₃alkylene-C₁₋₃alkoxy, C(═O)OC₁₋₃alkyl, C₀₋₄alkylene-cycloalkyl having 3-7 total ring atoms, C₀₋₄alkylene-heterocycloalkyl having 3-7 total ring atoms and 1, 2, or 3 heteroatoms selected from N, O, and S, C₀₋₄alkylene-aryl having 6-10 total ring atoms, C₀₋₄alkylene-heteroaryl having 5-7 total ring atoms and 1, 2, or 3 heteroatoms selected from N, O, and S; two geminal R₇ form CH₂ or an oxo group; two geminal R⁷ together with the atom to which they are attached from a spiro-cycloalkyl ring having 3-7 total ring atoms or a spiro-heterocycloalkyl ring having 4-7 total ring atoms and 1 or 2 heteroatoms selected from N, O and S; or two adjacent R⁷ together with the atoms to which they are attached form a fused cycloalkyl ring having 3-7 total ring atoms, a fused heterocycloalkyl ring having 3-7 total ring atoms and 1, 2, or 3 heteroatoms selected from N, O and S, a fused aryl ring having 6 total ring atoms, or a fused heteroaryl ring having 5-7 total ring atoms and 1, 2, or 3 heteroatoms selected from N, O, and S; wherein the cycloalkyl, heterocycloalkyl, aryl, or heteroaryl ring of any of the foregoing is unsubstituted or substituted with 1 or more substituents;
each of R^{A1} and R^{A2} independently is H, C₁₋₃alkyl, C₁₋₃haloalkyl, or cycloalkyl having 3-5 total ring atoms;
each R^{N1} independently is H or C₁₋₄alkyl.

Provided herein as Embodiment 2, is the compound or salt of Embodiment 1, wherein at least one of R^{1a}, R^{1b}, and R² is H or D.

Provided herein as Embodiment 3, is the compound or salt of Embodiment 2, wherein each of R^{1a}, R^{1b}, and R² independently is H or D.

Provided herein as Embodiment 4, is the compound or salt of Embodiment 3, wherein each of R^{1a}, R^{1b}, and R² independently is H.

Provided herein as Embodiment 5, is the compound or salt of Embodiment 3, wherein each of R^{1a}, R^{1b}, and R² independently is D.

Provided herein as Embodiment 6, is the compound or salt of Embodiment 1 or 2, wherein at least one of R^{1a}, R^{1b}, and R² is halo.

Provided herein as Embodiment 7, is the compound or salt of Embodiment 6, wherein each halo independently is Br, Cl, or F.

Provided herein as Embodiment 8, is the compound or salt of Embodiment 1 or 2, wherein at least one of R^{1a}, R^{1b}, and R² is C₁₋₄alkyl or C₁₋₄haloalkyl.

Provided herein as Embodiment 9, is the compound or salt of Embodiment 8, wherein at least one of R^{1a}, R^{1b}, and R² is CH₃, CH₂F, CHF₂, or CF₃.

Provided herein as Embodiment 10, is the compound or salt of Embodiment 1 or 2, wherein at least one of R¹, R^{1b}, and R² is C₁₋₂alkylene-OH, C₀₋₂alkylene-C₁₋₄alkoxy, C₀₋₂alkylene-C₁₋₄haloalkoxy, C₀₋₂alkylene-CN, or C₀₋₂alkylene-N(R^{N1})₂.

Provided herein as Embodiment 11, is the compound or salt of Embodiment 10, wherein each R^{N1} independently is H or CH₃.

Provided herein as Embodiment 12, is the compound or salt of Embodiment 11, wherein each R^{N1} independently is H.

Provided herein as Embodiment 13, is the compound or salt of Embodiment 10 or 11, wherein at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂.

Provided herein as Embodiment 14, is the compound or salt of Embodiment 1 or 2, wherein at least one of R^{1a}, R^{1b}, and R² is C₁₋₂alkylene-heterocycloalkyl having 3-6 total ring atoms and 1 or 2 heteroatoms selected from N, O, and S.

Provided herein as Embodiment 15, is the compound or salt of Embodiment 14, wherein the heterocycloalkyl is aziridinyl, azetidinyl, pyrrolidinyl, piperidinyl, or morpholinyl.

Provided herein as Embodiment 16, is the compound or salt of Embodiment 14 or 15, wherein at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl.

Provided herein as Embodiment 17, is the compound or salt of Embodiment 1 or 2, wherein R^{1b} and R² together with the carbon atoms to which they are attached from a

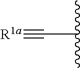

group.

Provided herein as Embodiment 18, is the compound or salt of Embodiment 1, wherein

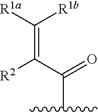

is

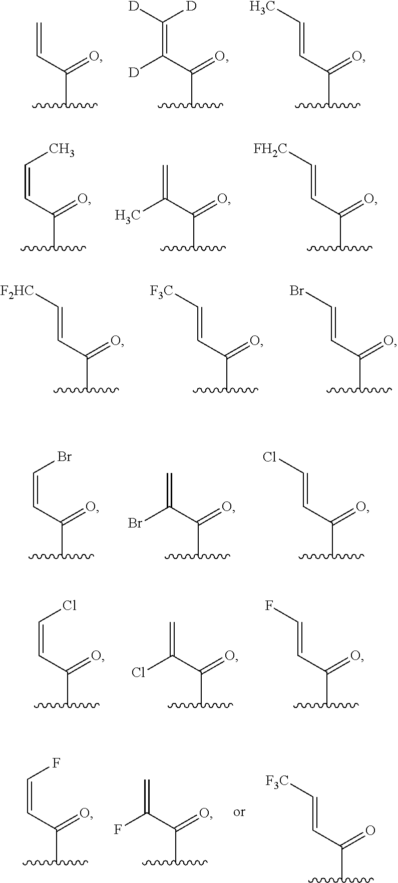

Provided herein as Embodiment 19, is the compound or salt of Embodiment 18, wherein

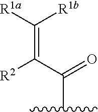

is

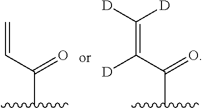

Provided herein as Embodiment 20, is the compound or salt of any one of Embodiments 1-19, wherein m is 0.

Provided herein as Embodiment 21, is the compound or salt of any one of Embodiments 1-19, wherein m is 1.

Provided herein as Embodiment 22, is the compound or salt of any one of Embodiments 1-19, wherein m is 2.

Provided herein as Embodiment 23, is the compound or salt of any one of Embodiments 1-19, wherein m is 3.

Provided herein as Embodiment 24, is the compound or salt of any one of Embodiments 1-19, wherein m is 4.

Provided herein as Embodiment 25, is the compound or salt of any one of Embodiments 1-24, wherein

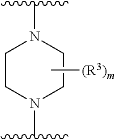

is deuterated.

Provided herein as Embodiment 26, is the compound or salt of Embodiment 25, wherein

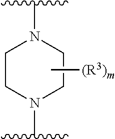

is

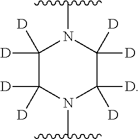

Provided herein as Embodiment 27, is the compound of salt of any one of Embodiments 21-24, wherein at least one R³ is C₁₋₃alkyl or C₁₋₃haloalkyl.

Provided herein as Embodiment 28, is the compound or salt of Embodiment 27, wherein at least one R³ is CH₃, CH₂CH₃, CF₃, CHF₂, or CH₂F.

Provided herein as Embodiment 29, is the compound or salt of any one of Embodiments 21-24, wherein at least one R³ is

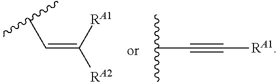

Provided herein as Embodiment 30, is the compound or salt of Embodiment 29, wherein each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl.

Provided herein as Embodiment 31, is the compound of Embodiment 29 or 30, wherein at least one R³ is

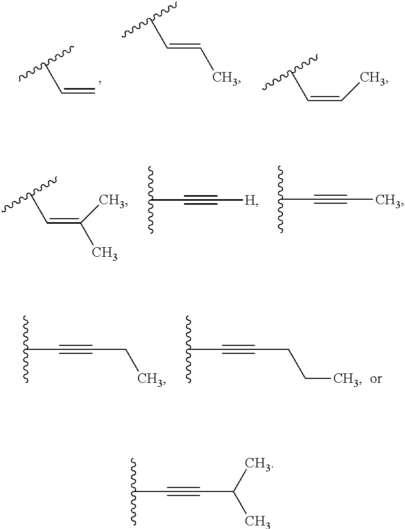

Provided herein as Embodiment 32, is the compound or salt of any one of Embodiments 21-24, wherein at least one R³ is C₀₋₃alkyleneCN.

Provided herein as Embodiment 33, is the compound or salt of Embodiment 32, wherein at least one R³ is CN or CH₂CN.

Provided herein as Embodiment 34, is the compound or salt of any one of Embodiments 21-24, wherein at least one R³ is C₀₋₃alkyleneOH or C₀₋₃alkylene-C₁₋₃alkoxy.

Provided herein as Embodiment 35, is the compound or salt of Embodiment 34, wherein at least one R³ is OH, CH₂OH, CH₂CH₂OH, OCH₃, CH₂OCH₃, or CH₂CH₂OCH₃.

Provided herein as Embodiment 36, is the compound or salt of any one of Embodiments 22-24, wherein two geminal R³ form an oxo group.

Provided herein as Embodiment 37, is the compound or salt of any one of Embodiments 22-24, wherein two geminal R³ together with the atom to which they are attached form a spiro-cycloalkyl ring having 3-7 total ring atoms or a spiro-heterocycloalkyl ring having 3-7 total ring atoms and 1 or 2 heteroatoms selected from N, O and S.

Provided herein as Embodiment 38, is the compound or salt of Embodiment 37, wherein two geminal R³ together with the atom to which they are attached form spiro-cyclopropyl, spiro-cyclobutyl, spiro-oxetanyl or spiro-tetrahydrofuranyl.

Provided herein as Embodiment 39, is the compound or salt of any one of Embodiments 22-24, wherein two adjacent R³ together with the atoms to which they are attached form a fused cycloalkyl ring having 3-7 total ring atoms.

Provided herein as Embodiment 40, is the compound or salt of Embodiment 39, wherein two adjacent R³ together with the atoms to which they are attached form a fused cyclopropyl ring or a fused cyclobutyl ring.

Provided herein as Embodiment 41, is the compound or salt of any one of Embodiments 1-19, wherein each R³ independently is CH₃, CH₂CH₃, CF₃, CHF₂, CH₂F, CN, CH₂CN, OH, CH₂OH, CH₂CH₂OH, OCH₃, CH₂OCH₃, or CH₂CH₂OCH₃; two geminal R³ form an oxo group; two geminal R³ together with the atom to which they are attached form spiro-cyclopropyl, spiro-cyclobutyl, spiro-oxetanyl, or spiro-tetrahydrofuranyl group; or two adjacent R³ together with the atoms to which they are attached form a fused cyclopropyl ring or a fused cyclobutyl ring.

Provided herein as Embodiment 42, is the compound or salt of any one of Embodiments 1-19, wherein m is 0; or m is 1 and R³ is CH₃, CF₃, CHF₂, CH₂F, CN, CH₂CN, CH₂OH, or CH₂OCH₃, or two geminal R³ together with the atom to which they are attached form spiro-oxetanyl.

Provided herein as Embodiment 43, is the compound or salt of any one of Embodiments 1-19, wherein

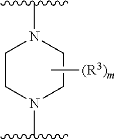

is

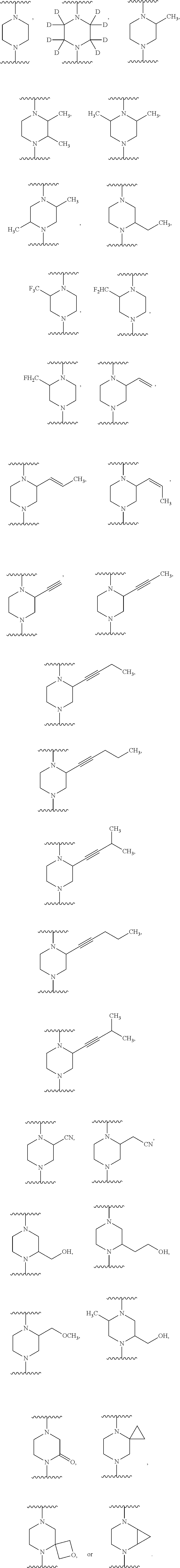

Provided herein as Embodiment 44, is the compound or salt of Embodiment 43, wherein

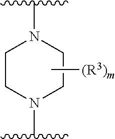

is

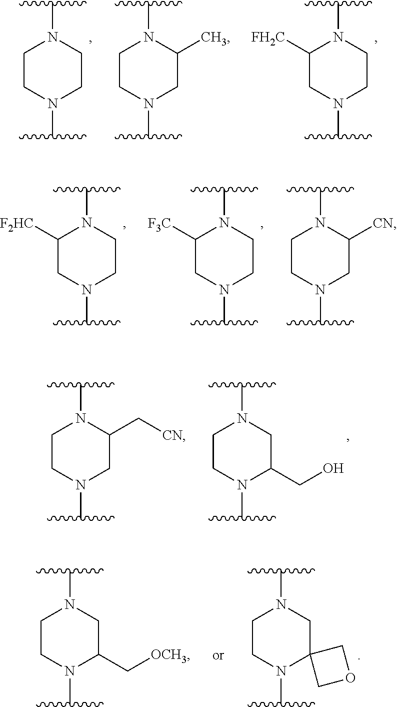

Provided herein as Embodiment 45, is the compound or salt of Embodiment 44, wherein

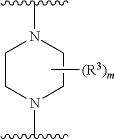

is

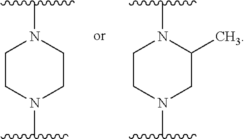 [SMILES: *N1CCN(*)CC1 ; "or" ; *N1CCN(*)C(C)C1]

Provided herein as Embodiment 46, is the compound or salt of any one of Embodiments 1-45, wherein A is N.

Provided herein as Embodiment 47, is the compound or salt of any one of Embodiments 1-45, wherein A is CH, C-halo, C—CN, C—C₁₋₃alkyl, C—C₁₋₃haloalkyl, C—C₀₋₃alkyleneOH, or C—C₀₋₃alkylene-C₁₋₄alkoxy.

Provided herein as Embodiment 48, is the compound or salt of Embodiment 47, wherein A is CH.

Provided herein as Embodiment 49, is the compound or salt of Embodiment 47, wherein A is C—F, C—Cl, or C—CN.

Provided herein as Embodiment 50, is the compound or salt of Embodiment 47, wherein A is C—C₁₋₃alkyl or C—C₁₋₃haloalkyl.

Provided herein as Embodiment 51, is the compound or salt of Embodiment 50, wherein A is C—CH₃, C—CH₂F, C—CHF₂, or C—CF₃.

Provided herein as Embodiment 52, is the compound or salt of Embodiment 47, wherein A is C—C₀₋₃alkylencOH or C—C₀₋₃alkylene-C₁₋₄alkoxy.

Provided herein as Embodiment 53, is the compound or salt of Embodiment 52, wherein A is C—OH, C—CH₂OH, C—OCH₃, or C—CH₂OCH₃.

Provided herein as Embodiment 54, is the compound or salt of any one of Embodiments 1-53, wherein n is 0.

Provided herein as Embodiment 55, is the compound or salt of any one of Embodiments 1-53, wherein n is 1.

Provided herein as Embodiment 56, is the compound or salt of any one of Embodiments 1-53, wherein n is 2.

Provided herein as Embodiment 57, is the compound or salt of Embodiment 55 or 56, wherein at least one R⁴ is C₁₋₃alkyl or C₁₋₃haloalkyl.

Provided herein as Embodiment 58, is the compound or salt of Embodiment 57, wherein at least one R⁴ is CH₃, CH₂CH₃, CH₂CH₂CH₃, CH(CH₃)₂, CF₃, CHF₂, or CH₂F.

Provided herein as Embodiment 59, is the compound or salt of Embodiment 55 or 56, wherein at least one R⁴ is C₀₋₃alkyleneCN.

Provided herein as Embodiment 60, is the compound or salt of Embodiment 59, wherein at least one R⁴ is CN or CH₂CN.

Provided herein as Embodiment 61, is the compound or salt of Embodiment 55 or 56, wherein at least one R⁴ is C₁₋₃alkyleneOH or C₁₋₃alkylene-C₁₋₃alkoxy.

Provided herein as Embodiment 62, is the compound or salt of Embodiment 61, wherein at least one R⁴ is CH₂OH, CH₂CH₂OH, CH₂OCH₃, or CH₂CH₂OCH₃.

Provided herein as Embodiment 63, is the compound or salt Embodiment 56, wherein two geminal R⁴ form an oxo group.

Provided herein as Embodiment 64, is the compound or salt of Embodiment 56, wherein two geminal R⁴ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S.

Provided herein as Embodiment 65, is the compound or salt of Embodiment 64, wherein two geminal R⁴ together with the atom to which they are attached form spiro-cyclopropyl, spiro-cyclobutyl, or spiro-oxetanyl.

Provided herein as Embodiment 66, is the compound or salt of Embodiment 46, wherein

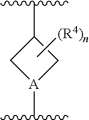

is

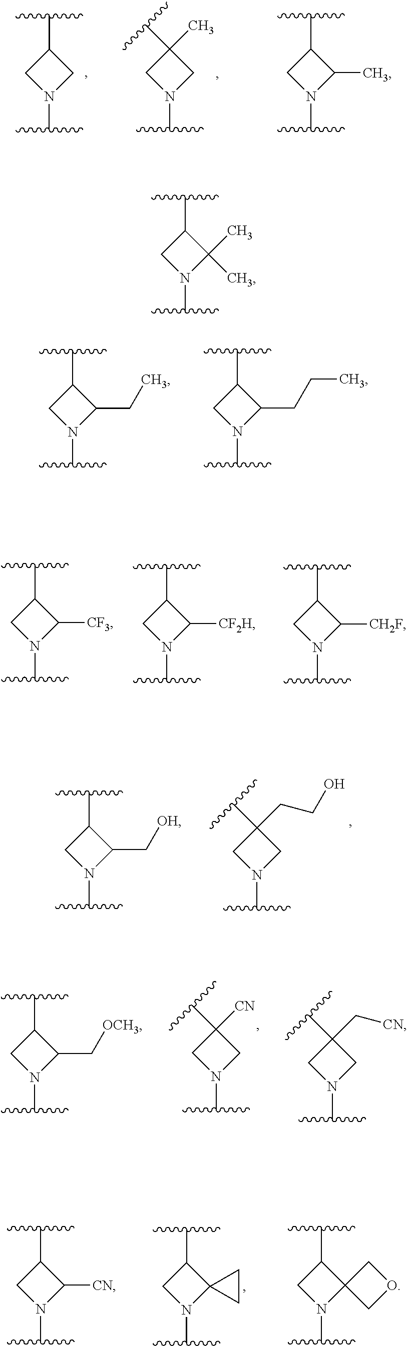

Provided herein as Embodiment 67, is the compound or salt of Embodiment 66, wherein

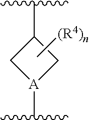

is

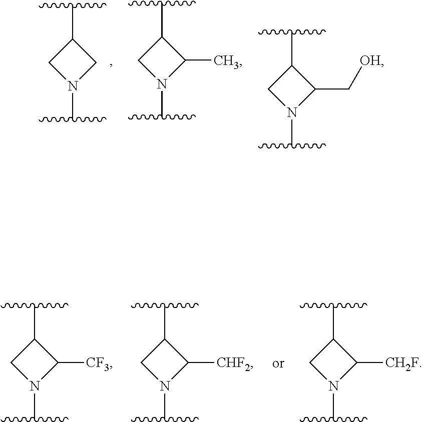

Provided herein as Embodiment 68, is the compound or salt of Embodiment 67, wherein

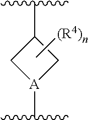

is

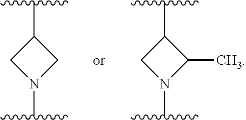 [SMILES: *C1CN(*)C1 ; *C1CN(*)C1C]

Provided herein as Embodiment 69, is the compound or salt of Embodiment 47, wherein

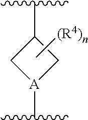

is

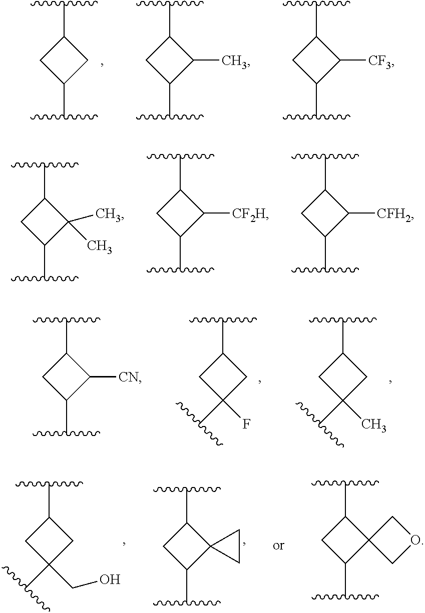

Provided herein as Embodiment 70, is the compound or salt of any one of Embodiments 1-69, wherein W is CH.

Provided herein as Embodiment 71, is the compound or salt of any one of Embodiments 1-69, wherein Wis C—F, C—Cl, or C—CN.

Provided herein as Embodiment 72, is the compound or salt of any one of Embodiments 1-69, wherein W is C—C₁₋₃alkyl or C—C₁₋₃haloalkyl.

Provided herein as Embodiment 73, is the compound or salt of Embodiment 72, wherein W is C—CH₃, C—CH₂CH₃, C—CH₂F, C—CHF₂, or C—CF₃.

Provided herein as Embodiment 74, is the compound or salt of any one of Embodiments 1-69, wherein W is C—C₀₋₃alkyleneOH or C—C₀₋₃alkylene-C₁₋₄alkoxy.

Provided herein as Embodiment 75, is the compound or salt of Embodiment 74, wherein W is C—OH, C—CH₂OH, C—OCH₃, or C—CH₂OCH₃.

Provided herein as Embodiment 76, is the compound or salt of any one of Embodiments 1-75, wherein X is N.

Provided herein as Embodiment 77, is the compound or salt of any one of Embodiments 1-75, wherein X is C—R^{5a}.

Provided herein as Embodiment 78, is the compound or salt of Embodiment 77, wherein R^{5a} is H.

Provided herein as Embodiment 79, is the compound or salt of Embodiment 77, wherein R^{5a} is CN.

Provided herein as Embodiment 80, is the compound or salt of Embodiment 77, wherein R^{5a} is Br, Cl, or F.

Provided herein as Embodiment 81, is the compound or salt of Embodiment 77, wherein R^{5a} is C₁₋₃alkyl or C₁₋₃haloalkyl.

Provided herein as Embodiment 82, is the compound or salt of Embodiment 81, wherein R^{5a} is CH₃, CH₂CH₃, CF₃, CHF₂, or CH₂F.

Provided herein as Embodiment 83, is the compound or salt of Embodiment 82, wherein R^{5a} is CH₃.

Provided herein as Embodiment 84, is the compound or salt of Embodiment 77, wherein R^{5a} is C₂₋₃alkenyl or C₂₋₃alkynyl.

Provided herein as Embodiment 85, is the compound or salt of Embodiment 84, wherein R^{5a} is

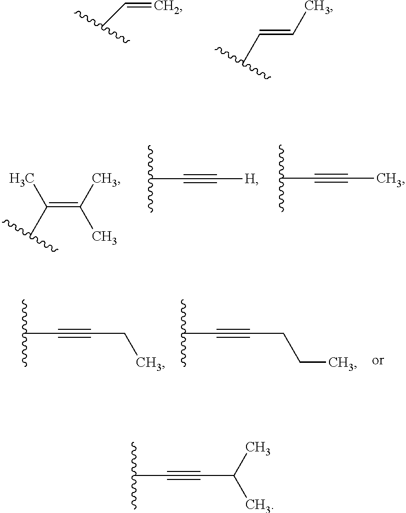

Provided herein as Embodiment 86, is the compound or salt of Embodiment 85, wherein R^{5a} is

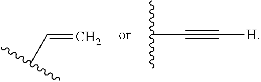

Provided herein as Embodiment 87, is the compound or salt of Embodiment 77, wherein R^{5a} is C₀₋₃alkyleneOH, C₀₋₃alkylene-C₁₋₃alkoxy, or cycloalkyl having 3-5 total ring atoms.

Provided herein as Embodiment 88, is the compound or salt of Embodiment 87, wherein R^{5a} is OH, CH₂OH, OCH₃, CH₂OCH₃, or

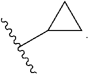 [SMILES: *C1CC1]

Provided herein as Embodiment 89, is the compound or salt of any one of Embodiments 1-88, wherein R^{5b} is C₁₋₃haloalkyl.

Provided herein as Embodiment 90, is the compound or salt of Embodiment 89, wherein R^{5b} is CF₃, CF₂H, CFH₂, or CF₂CH₃.

Provided herein as Embodiment 91, is the compound or salt of any one of Embodiments 1-88, wherein R^{5b} is Br, Cl, or F.

Provided herein as Embodiment 92, is the compound or salt of any one of Embodiments 1-88, wherein R^{5b} is C₁₋₃alkoxy or C₁₋₃thioalkoxy.

Provided herein as Embodiment 93, is the compound or salt of Embodiment 92, wherein R^{5b} is OCH₃, or SCH₃.

Provided herein as Embodiment 94, is the compound or salt of any one of Embodiments 1-88, wherein R^{5b} is C₁₋₆alkyl, C₂₋₄alkenyl, or C₂₋₄alkynyl; and each of the foregoing is independently unsubstituted or substituted with 1, 2, or 3 substituents selected from C₁₋₃haloalkyl, C₀₋₆alkylene(OH), C₀₋₆alkylene-C₁₋₃alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 3-7 total ring atoms and 1-3 heteroatoms selected from N, O, and S, and phenyl.

Provided herein as Embodiment 95, is the compound or salt of Embodiment 94 wherein each of the 1, 2, or 3 substituents independently is selected from CH₃, CF₃, CF₂H, CFH₂, OH, OCH₃, OCF₃, CH₂OH, CH₂OCH₃, cyclopropyl, cyclobutyl, and phenyl.

Provided herein as Embodiment 96, is the compound or salt of Embodiment 94 or 95, wherein R^{5b} is CH₃, CH₂CH₃, CH₂CH₂CH₃, CH(CH₃)₂,

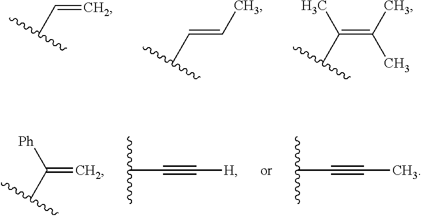

Provided herein as Embodiment 97, is the compound or salt of Embodiment 77, wherein R^{5a} and R^{5b} together with the atoms to which they are attached form a fused cycloalkyl ring having 3-7 total ring atoms.

Provided herein as Embodiment 98, is the compound or salt of Embodiment 97, wherein R^{5a} and R^{5b} together with the atoms to which they are attached form a fused-cyclopropyl, fused-cyclobutyl, or fused-cyclopentyl ring.

Provided herein as Embodiment 99, is the compound or salt of any one of Embodiments 1-69, wherein W is CH; X is N; and R^{5b} is CF₃, CF₂H, or CFH₂.

Provided herein as Embodiment 100, is the compound or salt of any one of Embodiments 1-69, wherein

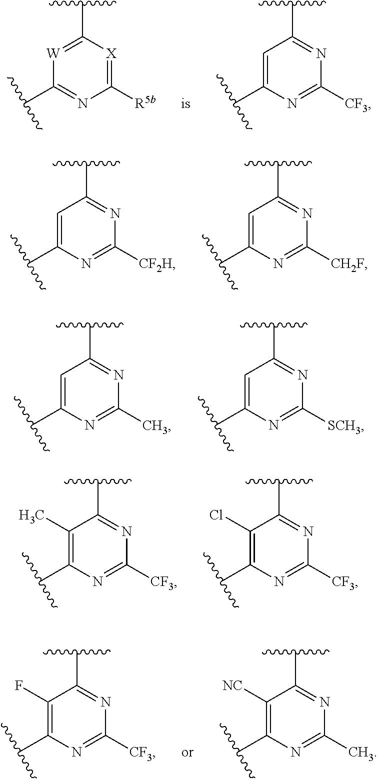

Provided herein as Embodiment 101, is the compound or salt of Embodiment 100, wherein

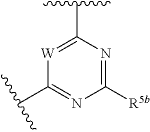

is

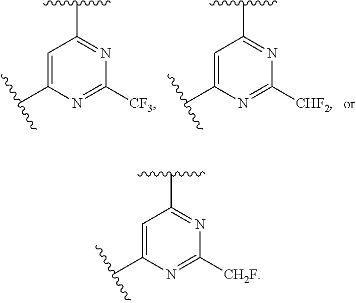

Provided herein as Embodiment 102, is the compound or salt of any one of Embodiments 1-69, wherein W is CH; X is C—R^{5a}, R^{5a} is CN, Br, Cl, F, or CH₃; and R^{5b} is CF₃, CF₂H, or CFH₂.

Provided herein as Embodiment 103, is the compound or salt of any one of Embodiments 1-69 wherein

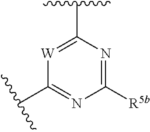

is

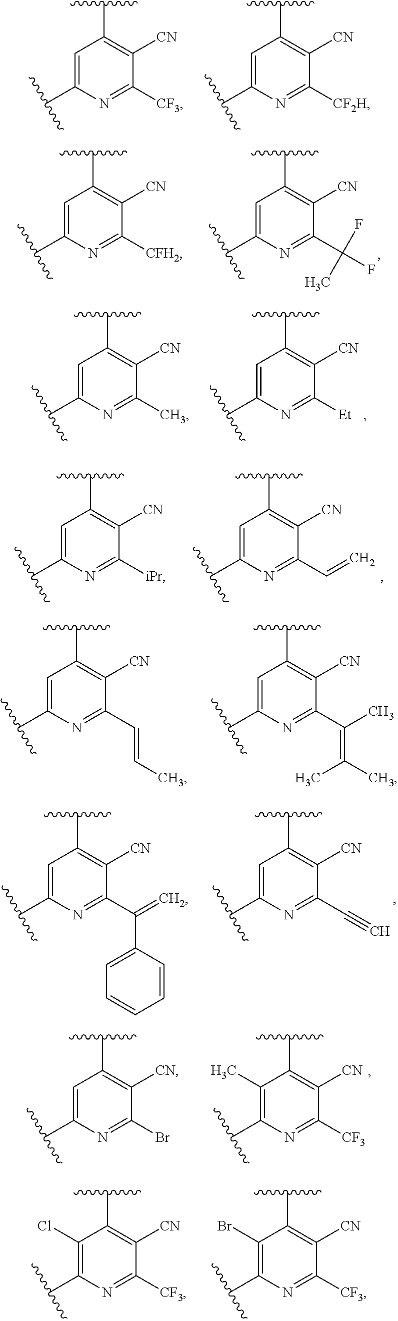

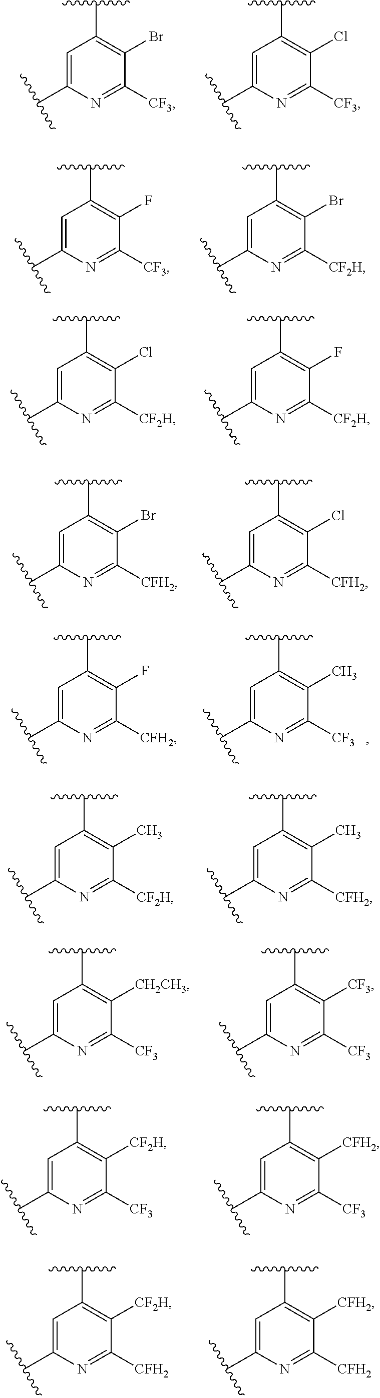

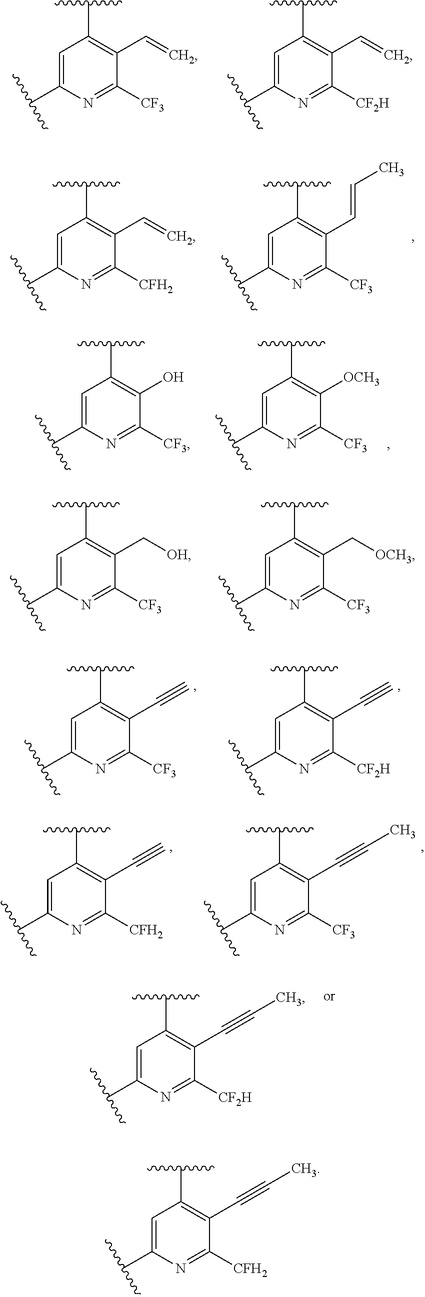

Provided herein as Embodiment 104, is the compound or salt of Embodiment 103, wherein

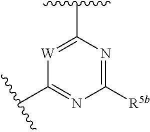

is

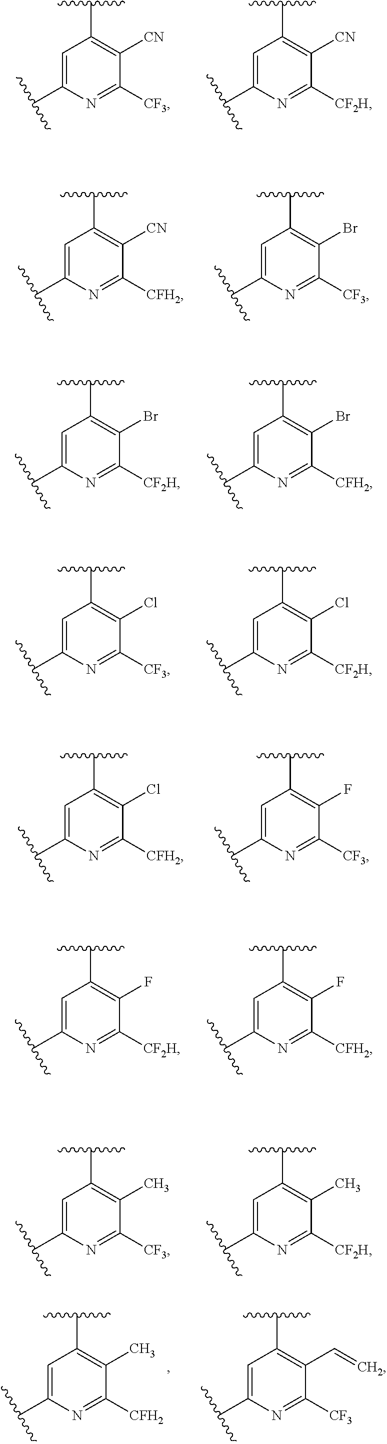

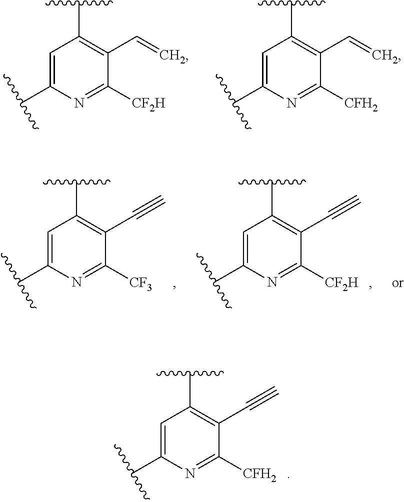

Provided herein as Embodiment 105, is the compound or salt of Embodiment 104, wherein

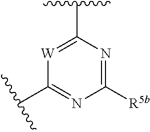

is

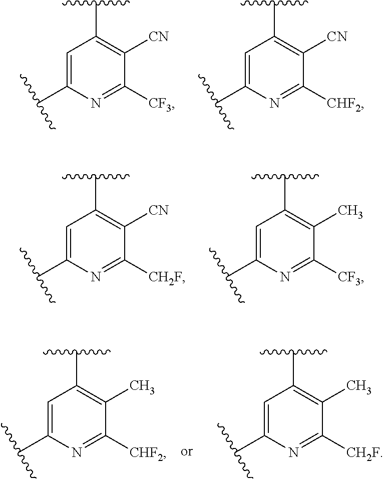

Provided herein as Embodiment 106, is the compound or salt of any one of Embodiments 1-105, wherein o is 0.

Provided herein as Embodiment 107, is the compound or salt of any one of Embodiments 1-105, wherein o is 1.

Provided herein as Embodiment 108, is the compound or salt of any one of Embodiments 1-105, wherein o is 2.

Provided herein as Embodiment 109, is the compound or salt of any one of Embodiments 1-105, wherein o is 3.

Provided herein as Embodiment 110, is the compound or salt of any one of Embodiments 1-105, wherein o is 4.

Provided herein as Embodiment 111, is the compound or salt of any one of Embodiments 107-110, wherein at least one R⁶ is Br, Cl, F, or CN.

Provided herein as Embodiment 112, is the compound or salt of Embodiment 111, wherein at least one R⁶ is F.

Provided herein as Embodiment 113, is the compound or salt of any one of Embodiments 107-110, wherein at least one R⁶ is C₁₋₃alkyl or C₁₋₃haloalkyl.

Provided herein as Embodiment 114, is the compound or salt of Embodiment 113, wherein at least one R⁶ is CH₃, CH₂F, CHF₂, or CF₃.

Provided herein as Embodiment 115, is the compound or salt of any one of Embodiments 107-110, wherein at least one R⁶ is C₀₋₃alkyleneOH, C₀₋₃alkylene-C₁₋₃alkoxy, deuterated C₀₋₃alkylene-C₁₋₃alkoxy, or C₁₋₄alkylene-N(R^{N1})₂, and each R^{N1} independently is H or CH₃.

Provided herein as Embodiment 116, is the compound or salt of Embodiment 115, wherein at least one R⁶ is OH, CH₂OH, OCH₃, OCD₃, CH₂OCH₃, or CH₂N(CH₃)₂.

Provided herein as Embodiment 117, is the compound or salt of any one of Embodiments 108-110, wherein two geminal R⁶ form an oxo group.

Provided herein as Embodiment 118, is the compound or salt of any one of Embodiments 108-110, wherein two geminal R⁶ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S.

Provided herein as Embodiment 119, is the compound or salt of Embodiment 118, wherein two geminal R⁶ together with the atom to which they are attached form spiro-cyclopropyl, spiro-cyclobutyl, spiro-oxetanyl, or spiro-tetrahydrofuranyl.

Provided herein as Embodiment 120, is the compound or salt of Embodiment 119, wherein two geminal R⁶ together with the atom to which they are attached form spiro-cyclopropyl.

Provided herein as Embodiment 121, is the compound or salt of any one of Embodiments 108-110, wherein two adjacent R⁶ together with the atoms to which they are attached form a fused cycloalkyl ring having 3-7 total ring atoms; wherein the fused cycloalkyl ring is unsubstituted or substituted with 1 or 2 substituents selected from halo, OH, C₁₋₃alkoxy, or CN.

Provided herein as Embodiment 122, is the compound or salt of Embodiment 121, wherein two adjacent R⁶ together with the atoms to which they are attached form a fused-cyclopropyl, a fused-cyclobutyl, or a fused-cyclopentyl ring.

Provided herein as Embodiment 123, is the compound or salt of any one of Embodiments 108-110, wherein two non-adjacent R⁶ join together to form a C₁₋₃alkylene bridge or a C₁₋₃ether bridge.

Provided herein as Embodiment 124, is the compound or salt of Embodiment 123, wherein two non-adjacent R⁶ join together to form —CH₂—, —CH₂CH₂—, —CH₂CH₂CH₂—, or —CH₂OCH₂—.

Provided herein as Embodiment 125, is the compound or salt of any one of Embodiments 1-105, wherein

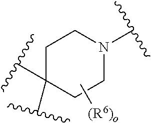

is

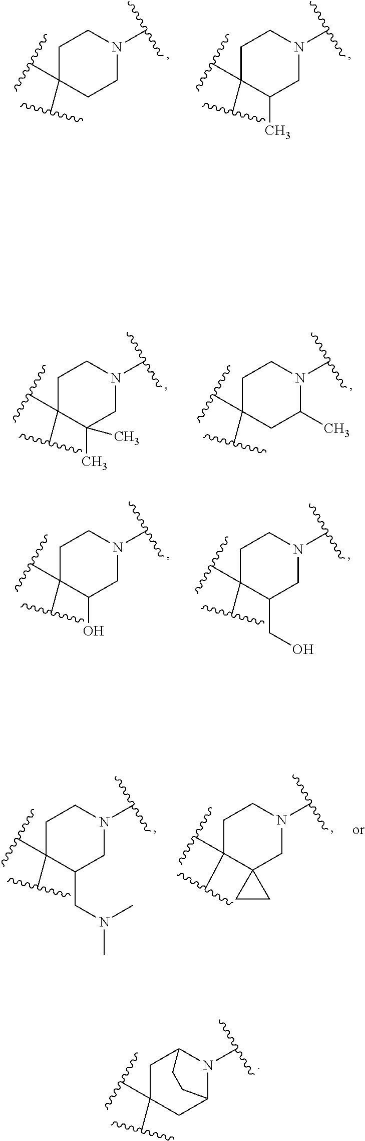

Provided herein as Embodiment 126, is the compound or salt of Embodiment 125, wherein

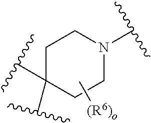

is

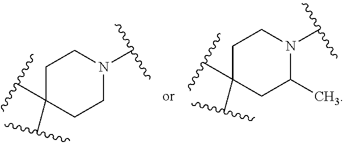 [SMILES: *N1CCC(*)(*)CC1 ; *N1CCC(*)(*)CC1C]

Provided herein as Embodiment 127, is the compound or salt of any one of Embodiments 1-126, wherein Y′ is cycloalkyl having 3 to 7 total ring atoms or cycloalkenyl having 4 to 7 total ring atoms.

Provided herein as Embodiment 128, is the compound or salt of Embodiment 127 wherein Y′ is cyclopropyl, cyclobutyl, cyclopentyl, cyclohexyl, cyclopentenyl, or cyclohexenyl.

Provided herein as Embodiment 129, is the compound or salt of any one of Embodiments 1-126, wherein Y′ is heterocycloalkyl having 3 to 7 total ring atoms and 1, 2, 3 heteroatoms selected from N, O, and S; or heterocycloalkenyl having 5 to 7 total ring atoms and 1, 2, or 3 heteroatoms selected from N, O, and S.

Provided herein as Embodiment 130, is the compound or salt of Embodiment 129, wherein Y′ is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothipyranyl, dithianyl, morpholinyl, thiomorpholinyl, dihydropyrrolyl, dihydrofuranyl, dihydrothiopheneyl, dihydroisoxazolyl, tetrahydropyridinyl, dihydropyranyl, or dihydrothipyranyl.

Provided herein as Embodiment 131, is the compound or salt of Embodiment 130, wherein Y′ is azetidinyl, oxetanyl, thietanyl, pyrrolidinyl, imidazolidinyl, tetrahydrofuranyl, oxazolidinyl, isoxazolidinyl, tetrahydropyranyl, dioxanyl, morpholinyl, dihydropyrrolyl, dihydrofuranyl, dihydroisoxazolyl, or dihydropyranyl.

Provided herein as Embodiment 132, is the compound or salt of Embodiment 131, wherein Y′ is pyrrolidinyl, tetrahydrofuranyl, tetrahydropyranyl, morpholinyl, dihydrofuranyl, dihydroisoxazolyl, or dihydropyranyl.

Provided herein as Embodiment 133, is the compound or salt of any one of Embodiments 1-132, wherein p is 0.

Provided herein as Embodiment 134, is the compound or salt of any one of Embodiments 1-132, wherein p is 1.

Provided herein as Embodiment 135, is the compound or salt of any one of Embodiments 1-132, wherein p is 2.

Provided herein as Embodiment 136, is the compound or salt of any one of Embodiments 1-132, wherein p is 3.

Provided herein as Embodiment 137, is the compound or salt of any one of Embodiments 1-132, wherein p is 4.

Provided herein as Embodiment 138, is the compound or salt of any one of Embodiments 1-132, wherein p is 5.

Provided herein as Embodiment 139, is the compound or salt of any one of Embodiments 134-138, wherein at least one R⁷ is F, Cl, Br, or CN.

Provided herein as Embodiment 140, is the compound or salt of any one of Embodiments 134-138, wherein at least one R⁷ is C₁₋₃alkyl, C₁₋₃haloalkyl, or C₂₋₃alkenyl.

Provided herein as Embodiment 141, is the compound or salt of Embodiment 140, wherein at least one R⁷ is CH₃, CH₂CH₃, CH₂F, CHF₂, or CF₃.

Provided herein as Embodiment 142, is the compound or salt of any one of Embodiments 134-138, wherein at least one R⁷ is C₀₋₃alkyleneOH or C₀₋₃alkylene-C₁₋₃alkoxy.

Provided herein as Embodiment 143, is the compound or salt of Embodiment 142, wherein at least one R⁷ is OH, OCH₃, OCH₂CH₃, CH₂OH, CH₂OCH₃, or CH₂CH₂OCH₃.

Provided herein as Embodiment 144, is the compound or salt of any one of Embodiments 134-138, wherein at least one R⁷ is C(═O)OC₁₋₃alkyl.

Provided herein as Embodiment 145, is the compound or salt of Embodiment 144, wherein at least one R⁷ is C(═O)OCH₃.

Provided herein as Embodiment 146, is the compound or salt of any one of Embodiments 134-138, wherein at least one R⁷ is C₀₋₄alkylene-cycloalkyl having 3-7 total ring atoms; C₀₋₄alkylene-heterocycloalkyl having 3-7 total ring atoms and 1, 2, or 3 heteroatoms selected from N, O, and S; C₀₋₄alkylene-aryl having 6-10 total ring atoms, or C₀₋₄alkylene-heteroaryl having 5-7 total ring atoms and 1, 2, or 3 heteroatoms selected from N, O, and S.

Provided herein as Embodiment 147, is the compound or salt of Embodiment 146, wherein at least one R⁷ is C₀₋₄alkylene-cyclopropyl, C₀₋₄alkylene-cyclobutyl, C₀₋₄alkylene-cyclopentyl, C₀₋₄alkylene-oxetanyl, C₀₋₄alkylene-pyrrolidinyl, C₀₋₄alkylene-phenyl, or C₀₋₄alkylene-oxadiazolyl.

Provided herein as Embodiment 148, is the compound or salt of any one of Embodiments 135-138, wherein two geminal R⁷ form CH₂ or an oxo group.

Provided herein as Embodiment 149, is the compound or salt of any one of Embodiments 135-138, wherein two geminal R⁷ together with the atom to which they are attached form a spiro-cycloalkyl ring having 3-7 total ring atoms or a spiro-heterocycloalkyl ring having 4-7 total ring atoms and 1 or 2 heteroatoms selected from N, O and S.

Provided herein as Embodiment 150, is the compound or salt of Embodiment 149, wherein two geminal R together with the atom to which they are attached form spiro-cyclopropyl, spiro-cyclobutyl, spiro-oxetanyl, or spiro-tetrahydrofuranyl.

Provided herein as Embodiment 151, is the compound or salt of any one of Embodiments 135-138, wherein two adjacent R⁷ together with the atoms to which they are attached form a fused cycloalkyl ring having 3-7 total ring atoms or a fused heterocycloalkyl ring having 3-7 total ring atoms and 1, 2, or 3 heteroatoms selected from N, O, and S; wherein each of the foregoing is unsubstituted or substituted with 1, 2, or 3 substituents.

Provided herein as Embodiment 152, is the compound or salt of Embodiment 151, wherein two adjacent R⁷ together with the atoms to which they are attached form fused-cyclopropyl or fused-cyclobutyl.

Provided herein as Embodiment 153, is the compound or salt of any one of Embodiments 135-138, wherein two adjacent R⁷ together with the atoms to which they are attached form a fused aryl ring having 6 total ring atoms, or a fused heteroaryl ring having 5-7 total ring atoms and 1, 2, or 3 heteroatoms selected from N, O, and S; wherein each of the foregoing is unsubstituted or substituted with 1, 2, or 3 substituents.

Provided herein as Embodiment 154, is the compound or salt of Embodiment 153, wherein two adjacent R⁷ together with the atoms to which they are attached form fused-phenyl, fused-thiophenyl, fused-pyrazolyl, fused-thiazolyl, fused-pyridyl, or fused-pyridazinyl.

Provided herein as Embodiment 155, is the compound or salt of any one of Embodiments 146-147 and 149-154, wherein the cycloalkyl, heterocycloalkyl, aryl, and heteroaryl 1 ring is unsubstituted.

Provided herein as Embodiment 156, is the compound or salt of any one of Embodiments 146-147 and 149-154, wherein the cycloalkyl, heterocycloalkyl, aryl, and heteroaryl rings is substituted with 1, 2, or 3 substituents.

Provided herein as Embodiment 157, is the compound or salt of Embodiment 156, wherein each substituent independently is halo, CN, oxo, or C₁₋₃alkyl.

Provided herein as Embodiment 158, is the compound or salt of Embodiment 157, wherein each substituent independently is F, Cl, Br, CN, oxo, or CH₃.

Provided herein as Embodiment 159, is the compound or salt of any one of Embodiments 1-126, wherein

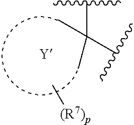

is

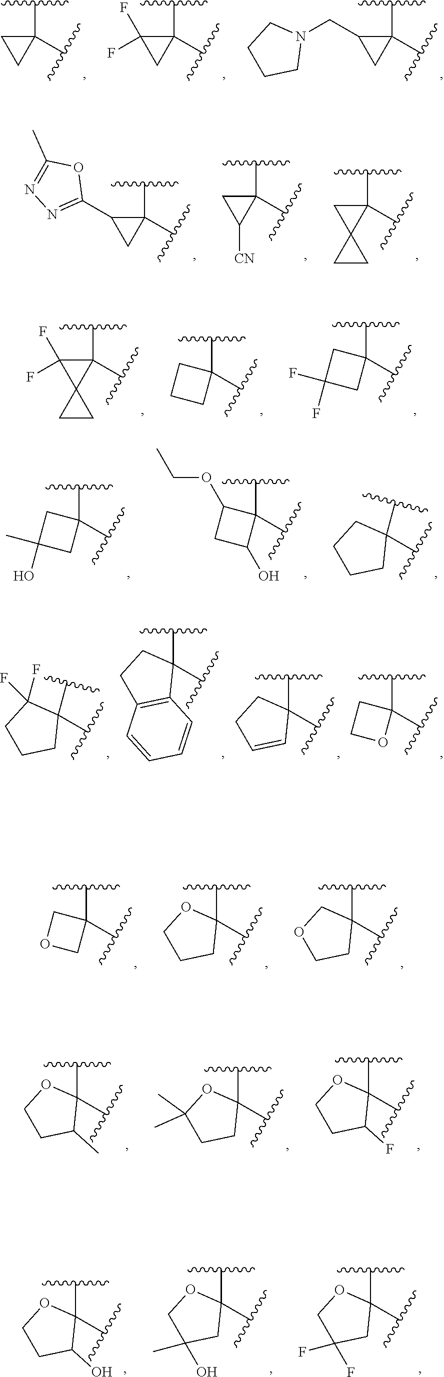

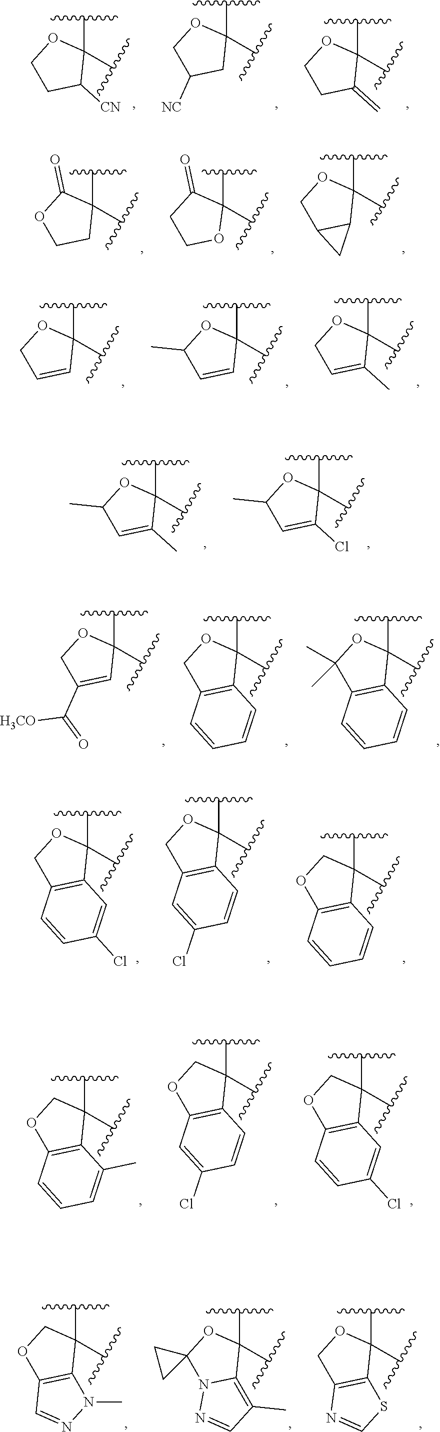

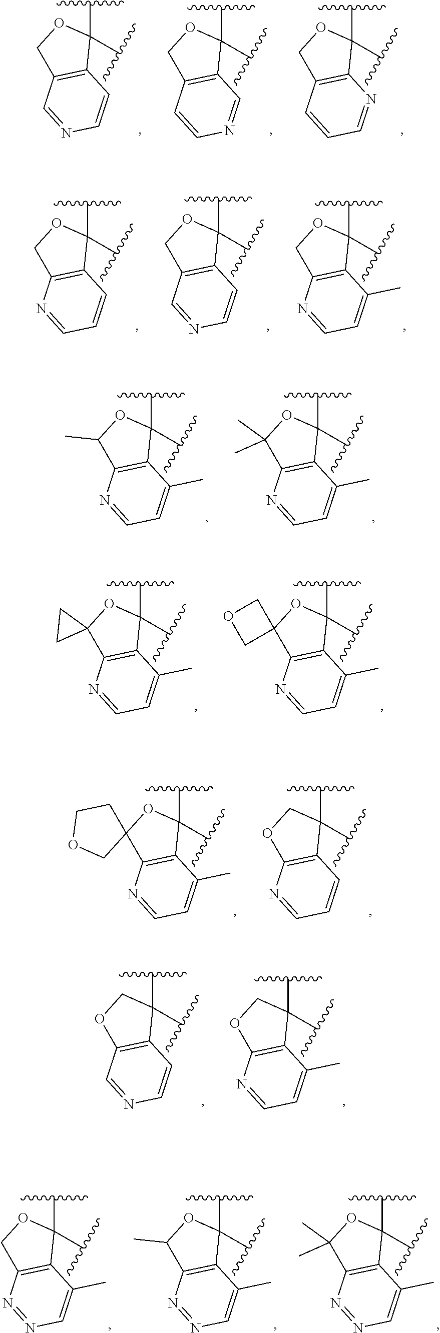

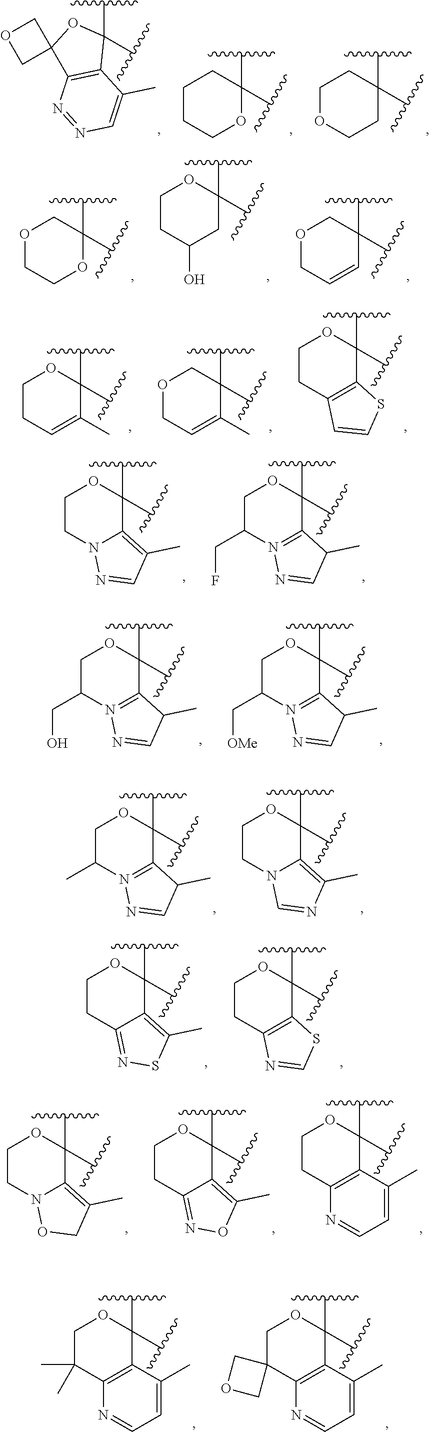

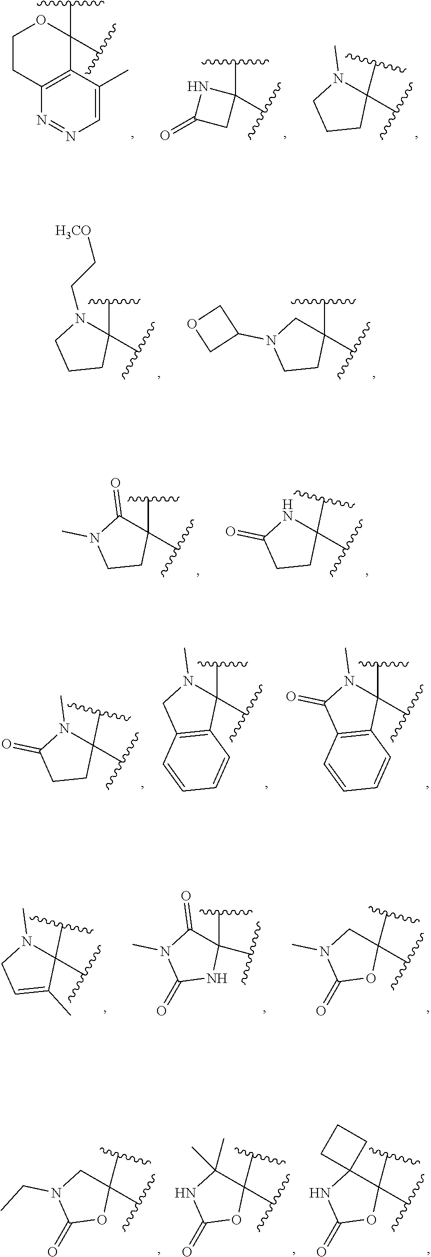

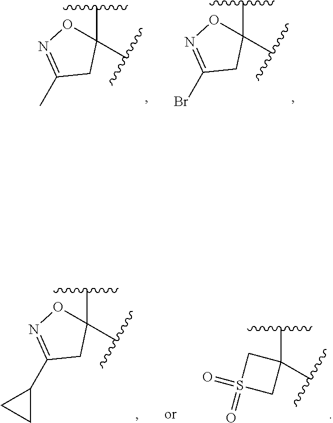

Provided herein as Embodiment 160, is the compound or salt of Embodiment 159, wherein

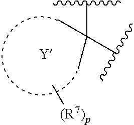

is

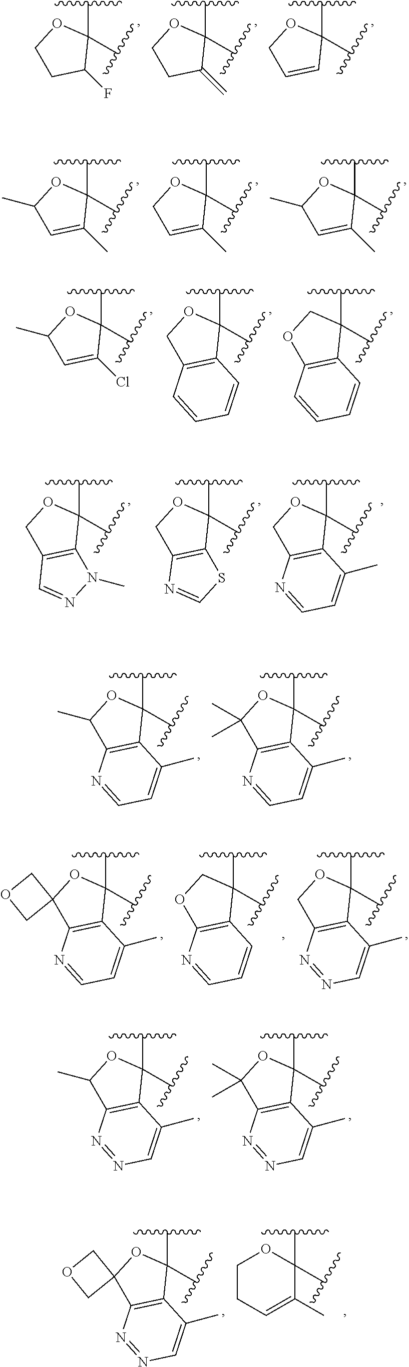

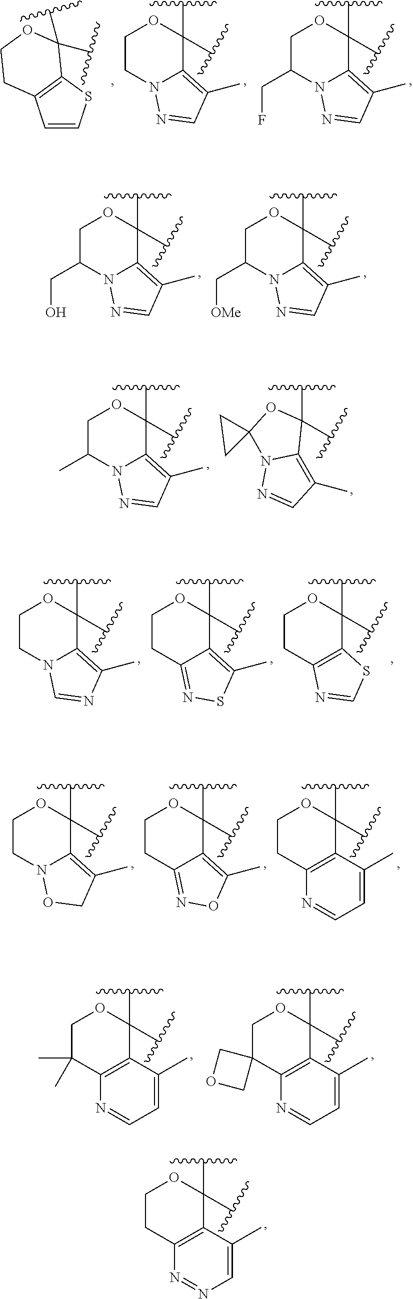

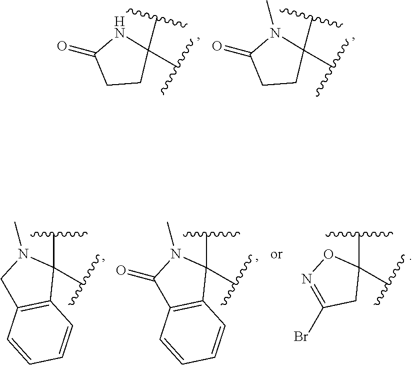

Provided herein as Embodiment 161, is the compound or salt of Embodiment 1, wherein:

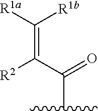

is

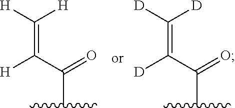 [SMILES: *C(=O)C=C ; "or" ; *C(=O)C([2H])=C([2H])[2H]]

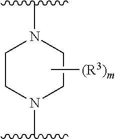

is

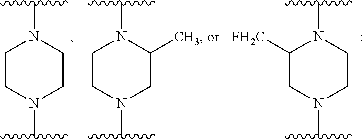

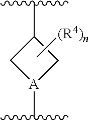

is

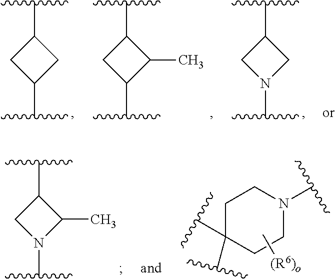

is

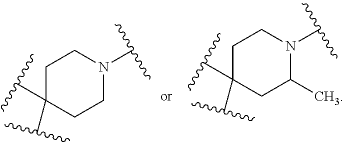 [SMILES: *N1CCC(*)(*)CC1 ; *N1CCC(*)(*)CC1C]

Provided herein as Embodiment 162, is the compound of Embodiment 161, wherein

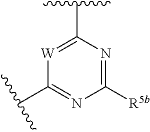

is

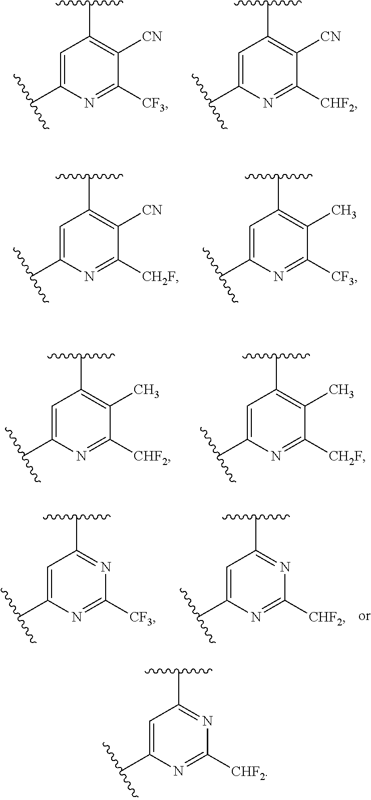

Provided herein as Embodiment 163, is the compound or salt of Embodiment 161 or 162, wherein

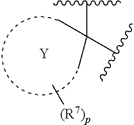

is

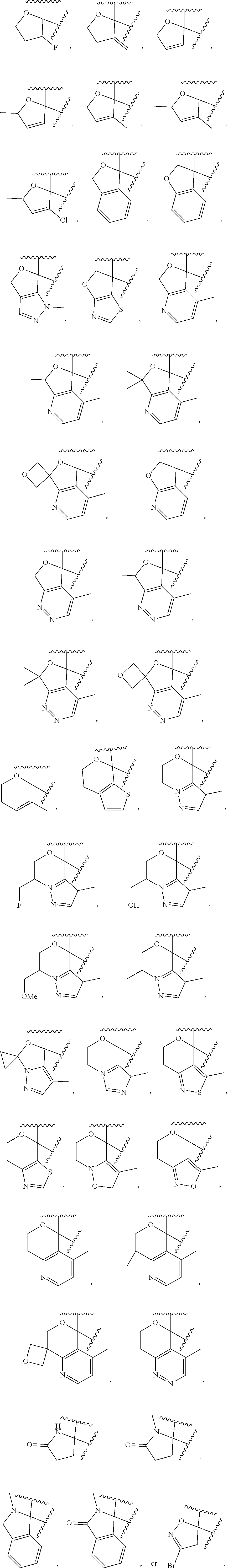

Provided herein as Embodiment 164, is the compound or salt of Embodiment 1, wherein the compound is a compound of Formula (IA) or Formula (IB);

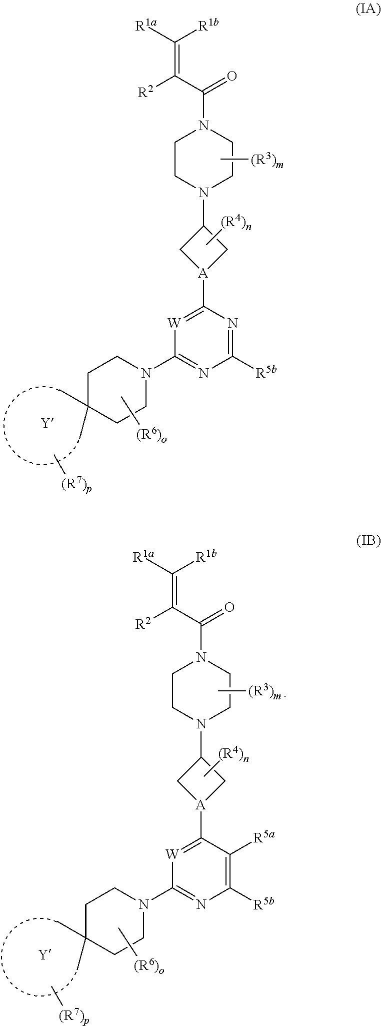

or a pharmaceutically acceptable salt of any of the foregoing.

Provided herein as Embodiment 165, is the compound of Embodiment 1, wherein the compound is a compound listed in Table A, or a pharmaceutically acceptable salt thereof.

Provided herein as Embodiment 166, is the compound of Embodiment 1, wherein the compound is a compound listed in Table B, or a pharmaceutically acceptable salt thereof.

Provided herein as Embodiment 167, is the compound of Embodiment 1, wherein the compound is a compound listed in Table C, or a pharmaceutically acceptable salt thereof.

Provided herein as Embodiment 168, is the compound of Embodiment 1, wherein the compound is a compound listed in Table D, or a pharmaceutically acceptable salt thereof.

Provided herein as Embodiment 169, is a pharmaceutical composition comprising the compound or salt of any one of Embodiments 1-168 and a pharmaceutically acceptable excipient.

Provided herein as Embodiment 170, is the compound or salt of any one of Embodiments 1-168, or the pharmaceutical composition of Embodiment 169 for use as a medicament.

Provided herein as Embodiment 171, is the compound or salt of any one of Embodiments 1-168, or the pharmaceutical composition of Embodiment 169 for use in treating cancer.

Provided herein as Embodiment 172, is the compound or salt of any one of Embodiments 1-168, or the pharmaceutical composition of Embodiment 169 for use in treating cancer, wherein one or more cells express KRAS G12C mutant protein.

Provided herein as Embodiment 173, is the compound, salt, or pharmaceutical composition for use of Embodiment 171 or 172, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 174, is a use of a compound or salt of any one of Embodiments 1-168, or the pharmaceutical composition of Embodiment 169 in the preparation of a medicament for treating cancer.

Provided herein as Embodiment 175, is a use of a compound or salt of any one of Embodiments 1-168, or the pharmaceutical composition of Embodiment 169 in the preparation of a medicament for treating cancer, wherein one or more cells express KRAS G12C mutant protein.

Provided herein as Embodiment 176, is the use of Embodiment 174 or 175, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 177, is a method of treating cancer in a subject in need thereof, the method comprising administering to the subject a therapeutically effective amount of the compound or salt of any one of Embodiments 1-168, or the pharmaceutical composition of Embodiment 169.

Provided herein as Embodiment 178, is the method of Embodiment 177, wherein one or more cells express KRAS G12C mutant protein.

Provided herein as Embodiment 179, is the method of Embodiment 177 or 178, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 180, is the method of Embodiment 179, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor.

Provided herein as Embodiment 181, is the method of Embodiment 180, wherein the cancer is non-small cell lung cancer.

Provided herein as Embodiment 182, is the method of Embodiment 180, wherein the cancer is colorectal cancer.

Provided herein as Embodiment 183, is the method of Embodiment 180, wherein the cancer is pancreatic cancer.

Provided herein as Embodiment 184, is the method of Embodiment 180, wherein the cancer is solid tumor.

Provided herein as Embodiment 185, is the method according to any one of Embodiments 177-184, wherein the subject has a cancer that was determined to have one or more cells expressing the KRAS G12C mutant protein prior to administration of the compound, salt, or pharmaceutical composition.

Provided herein as Embodiment 186, is the method according to any one of Embodiments 177-185, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

Provided herein as Embodiment 187, is the compound or salt of any one of Embodiments 1-186, wherein the compound or salt has an IC₅₀ value of less than 1 μM in the coupled exchange assay.

Provided herein as Embodiment 188, is a compound of Formula (B):

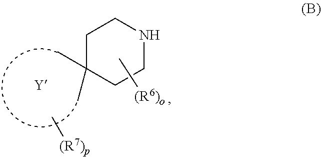

or a nitrogen-protected analog, or a pharmaceutically acceptable salt of any of the foregoing, wherein:
o is 0, 1, 2, 3, or 4;
p is 0, 1, 2, 3, 4, or 5;
each R⁶ independently is halo, CN, C₁₋₃alkyl, C₁₋₃haloalkyl, C₀₋₃alkyleneOH, C₀₋₃alkylene-C₁₋₃alkoxy, deuterated C₀₋₃alkylene-C₁₋₃alkoxy, or C₁₋₄alkylene-N(R^{N1})₂; two geminal R⁶ form oxo; two geminal R⁶ together with the atom to which they are attached from a spiro-cycloalkyl ring having 3-7 total ring atoms or a spiro-heterocycloalkyl ring having 4-7 total ring atoms and 1 or 2 heteroatoms selected from N, O and S; or two adjacent R⁶ together with the atoms to which they are attached form a fused cycloalkyl ring having 3-7 total ring atoms; wherein the cycloalkyl or heterocycloalkyl ring of any of the foregoing is unsubstituted or substituted with 1 or more substituents; or two non-adjacent Rejoin together to form a C₁₋₃alkylene bridge or a C₁₋₃ether bridge;
each R⁷ independently is halo, CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, C₀₋₄alkylene-cycloalkyl having 3-7 total ring atoms, C₀₋₄alkylene-heterocycloalkyl having 3-7 total ring atoms and 1, 2, or 3 heteroatoms selected from N, O, and S, C₀₋₄alkylene-aryl having 6-10 total ring atoms, C₀₋₄alkylene-heteroaryl having 5-7 total ring atoms and 1, 2, or 3 heteroatoms selected from N, O, and S; two geminal R⁷ form CH₂ or an oxo group; two geminal R⁷ together with the atom to which they are attached from a spiro-cycloalkyl ring having 3-7 total ring atoms or a spiro-heterocycloalkyl ring having 4-7 total ring atoms and 1 or 2 heteroatoms selected from N, O and S; or two adjacent R⁷ together with the atoms to which they are attached form a fused cycloalkyl ring having 3-7 total ring atoms, a fused heterocycloalkyl ring having 3-7 total ring atoms and 1, 2, or 3 heteroatoms selected from N, O and S, a fused aryl ring having 6 total ring atoms, or a fused heteroaryl ring having 5-7 total ring atoms and 1, 2, or 3 heteroatoms selected from N, O, and S; wherein the cycloalkyl, heterocycloalkyl, aryl, or heteroaryl ring of any of the foregoing is unsubstituted or substituted with 1 or more substituents; and
each R^{N1} independently is H or C₁₋₄alkyl.

Provided herein as Embodiment 189, is the compound of Embodiment 187, wherein the compound is a compound listed in Table E, a nitrogen-protected analog thereof, or a pharmaceutically acceptable salt of any of the foregoing.

Provided herein as Embodiment 190, is the compound of Embodiment 189, wherein the compound is compound B-1 to B-26, a nitrogen-protected analog thereof, a nitrogen-deprotected analog thereof, or a pharmaceutically acceptable salt of any of the foregoing.

Provided herein as Embodiment 191, is a method of preparing the compound or salt of any one of Embodiments 1-168, comprising admixing a compound of Formula (B):

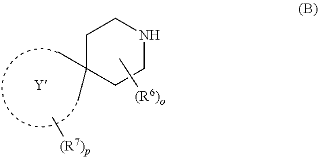

with a compound:

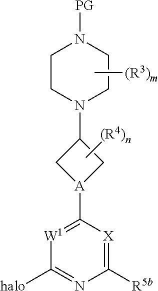

under carbon-nitrogen bond-forming conditions to form:

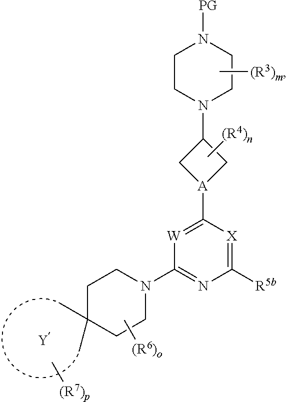

wherein PG is a protecting group.

### EXAMPLES

This section provides specific examples of compounds of Formulae (I) and (II) and methods of making the same.

TABLE 1
List of Abbreviations

| | |
|---|---|
| Ac | acetyl |
| ACN or MeCN | acetonitrile |
| AcOH | acetic acid |
| AIBN | azobisisobutyronitrile |
| anh or anhy | anhydride |
| aq or aq. | aqueous |
| Bn | benzyl |
| B₂pin₂ | bis(pinacolato)diboron |
| BOC or Boc | tert-butyloxycarbonyl |
| Bu | butyl |
| n-BuLi | n-butyllithium |
| Cbz | benzyloxycarbonyl |
| CbzCl | benzyl chloroformate |
| COD | cyclooctadiene |
| CV | column volume |
| DAST | diethy laminosulfur trifluoride |
| DBU | 1,8-diazabicyclo[5,4,0]undec-7-ene |
| DCE | dichloroethane |
| DCM | dichloromethane |
| Deoxo-Fluor | bis(2-methoxyethyl)aminosulfur trifluoride |
| DIAD | diisopropyl azodicarboxylate |
| DMA | N,N-dimethylacetamide |
| DMF | N,N-dimethylformamide |
| DMAP | N,N-dimethylpyridin-4-amine |
| DMSO | dimethyl sulfoxide |
| DMTMM | 4-(4,6-dimethoxy-1,3,5-triazin-2-yl)-4-methylmorpholin-4-ium |
| | chloride |
| DPPF or dppf | bis(diphenylphosphino)ferrocene |
| EDC | 1-ethyl-3-(3-dimethylaminopropyl)carbodiimide |
| EDTA | ethylenediaminetetraacetic acid |
| eq or eq. or equiv. | equivalent |
| ESI or ES | electrospray ionization |
| Et | ethyl |
| EtOAc | ethyl acetate |
| EtOH | ethanol |
| g or gr | gram(s) |
| h | hour(s) |
| HBpin | 4,4,5,5-tetramethyl-1,3,2-dioxaborolane |
| HPLC | high pressure liquid chromatography |
| iPr | iso-propyl |
| iPrOH or IPA | iso-propanol |
| iPr₂NEt or DIPEA | N-ethyl diisopropylamine (Hunig's base) |
| KHMDS | potassium hexamethyldisilazide |
| KOAc | potassium acetate |
| KOtBu | potassium tert-butoxide |
| LAH | lithium aluminum hydride |
| LC MS, LCMS, LC-MS or | liquid chromatography mass spectroscopy |
| LC/MS | |
| LDA | lithium diisopropylamide |
| LiHMDS | lithium hexamethyldisilazide |
| m/z | mass divided by charge |
| mCPBA | meta-chloroperoxybenzoic acid |
| Me | methyl |
| MeOH | methanol |
| mg | milligram(s) |
| min | minute(s) |
| mL | milliliter(s) |
| MS | mass spectra |
| MsCl | methanesulfonyl chloride |
| MTBE | methyl tert-butyl ether |
| NaHMDS | sodium hexamethyldisilazide |
| NIS | N-iodosuccinimide |
| NMR | nuclear magnetic resonance |
| Pd(dppf)Cl₂ | [1,1′-bis(diphenylphosphino)ferrocene]dichloropalladium(II) |
| Pd(dppf)Cl₂•DCM, | [1,1′-bis(diphenylphosphino)ferrocene]dichloropalladium(II) |
| | complex with dichloromethane |
| Pd(PPh₃)₄ | tetrakis(triphenylphosphine)palladium(0) |
| Ph | phenyl |
| PhMe | toluene |
| Pin | pinacolato |
| PMB | 4-methoxybenzyl |
| PTSA or pTsOH | p-toluenesulfonic acid |
| Rbf or rbf | round-bottomed flask |
| RP-HPLC | reverse phase high pressure liquid chromatography |
| rt or RT or r.t. | room temperature |
| RuPhos | 2-dicyclohexylphosphino-2′,6′-diisopropoxy-1,1′-biphenyl, |
| | dicyclohexyl(2′,6′-diisopropoxy-[1,1′-biphenyl]-2-yl)phosphine. |
| RuPhos Pd G1 MTBE | chloro-(2-dicyclohexylphosphino-2′,6′-diisopropoxy-1,1′- |
| | biphenyl)[2-(2-aminoethyl)phenyl]palladium(II) - methyl-t- |
| | butyl ether adduct |
| RuPhos Pd G3 | (2-dicyclohexylphosphino-2′,6′-diisopropoxy-1,1′-biphenyl)[2- |
| | (2′-amino-1,1′-biphenyl)]palladium (II) methanesulfonate |
| sat. or satd | saturated |
| SFC | supercritical fluid chromatography |
| SPhos | 2-dicyclohexylphosphino-2′,6′-dimethoxybiphenyl |
| SPhos Pd G3 | (2-dicyclohexylphosphino-2′,6′-dimethoxybiphenyl) [2-(2′- |
| | amino-1,1′-biphenyl)]palladium(II) methanesulfonate |
| SPhos Pd G4 | (methanesulfonato-κO)[2′-(methylamino)-2- |
| | biphenylyl]palladium - dicyclohexyl(2′,6′-dimethoxy-2- |
| | biphenylyl)phosphine |
| TBAF | tetra-n-butylammonium fluoride |
| TBS | tert-butyldimethylsilyl |
| TBSCl or TBDMSCl | tert-butyldimethylsilyl chloride |
| tBu | tert-butyl |
| TBHP | tert-butyl hydroperoxide |
| TEA or Et₃N | triethylamine |
| temp | temperature |
| Tf | trifluoromethylsulfonyl |
| TFA | trifluoroacetic acid |
| THF | tetrabydrofuran |
| TLC | thin layer chromatography |
| TsCl | 4-toluenesulfonyl chloride |
| UV | ultraviolet |

Provided in this section are descriptions of the general analytical and purification methods used to prepare the specific examples provided herein.

Chromatography: Unless otherwise indicated, crude product-containing residues were purified by passing the crude material or concentrate through either a Biotage or ISCO brand silica gel column pre-packed with flash silica and eluting the product off the column with a solvent gradient as indicated.

Preparative HPLC Method: Where indicated, the compounds described herein were purified via reverse phase HPLC using Waters FractionLynx or Gilson semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% ACN (0.1% v/v formic acid) in H₂O (0.1% v/v formic acid) or 10% (v/v) to 100% ACN (0.1% v/V TFA) in H₂O (0.1% v/v TFA) over 10 min. Conditions can be varied to achieve optimal separations.

Proton NMR Spectra: Unless otherwise indicated, all 1H NMR spectra were collected on a Bruker NMR instrument at 300, 400 or 500 MHz. All observed protons are reported as parts-per-million (ppm) downfield from tetramethylsilane (TMS) using the internal solvent peak as reference. Some ¹H signals may be missing due to exchange with D from CD₃OD, or due to signal suppression.

Fluorine-19 NMR Spectra: Unless otherwise indicated, all ¹⁹F NMR spectra were collected on a Bruker NMR instrument at 300 or 400 MHz.

Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

If the stereochemistry of a structure or a portion of a structure in the section below is not explicitly shown (e.g., such as with dashed or bold lines), then the structure or portion of structure is either achiral or interpreted as being any of the possible stereoisomers of the structure or portion of the structure. If the stereochemistry of a structure or portion of a structure in the section below is explicitly shown, a single stereoisomer of the structure or portion of the structure is represented, with the understanding that the stereochemistry of the structure or portion of the structure has been arbitrarily assigned.

### Section 1: Synthesis of Intermediates

### Spirocyclic Intermediates

In some cases, the spirocyclic intermediates described herein are prepared as TFA salts.

### 1-Oxa-(8-azaspiro[4.5]decan-6-yl)methanol (Intermediate B-1)

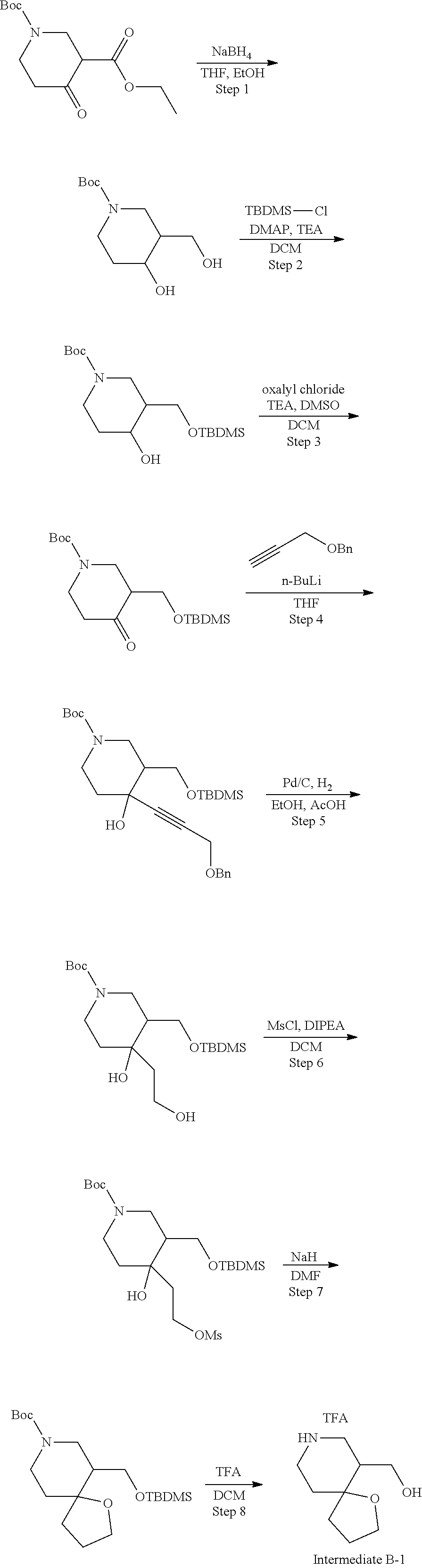

Step 1: tert-Butyl 4-hydroxy-3-(hydroxymethyl)piperidine-1-carboxylate. To a solution of 1-(tert-butyl) 3-ethyl 4-oxopiperidine-1,3-dicarboxylate (15 g, 55.3 mmol) in THE (150 mL) and EtOH (30 mL) at 0° C. was added sodium borohydride (6.27 g, 166 mmol) and the mixture was stirred at rt for 12 h. The reaction mixture was diluted with H₂O and extracted with EtOAc (3×100 mL). The combined organic extracts were dried over Na₂SO₄ and concentrated to give tert-butyl 4-hydroxy-3-(hydroxymethyl)piperidine-1-carboxylate. MS (ESI) m/z: 176.1 (M+H−56)⁺.

Step 2: tert-Butyl 3-(((tert-butyldimethylsilyl)oxy)methyl)-4-hydroxypiperidine-1-carboxylate. To a solution of tert-butyl 4-hydroxy-3-(hydroxymethyl)piperidine-1-carboxylate (12 g, 51.9 mmol), DMAP (1.27 g, 10.38 mmol) and TEA (14.46 mL, 104 mmol) in DCM (120 mL) at 0° C. was added TBDMS-Cl (8.60 g, 57.1 mmol) and stirred for 16 h at rt. After 16 h, the reaction mixture was quenched with H₂O (500 mL) and extracted with DCM (2×100 mL). The combined organic extracts were washed with H₂O (500 mL), brine (500 mL), dried over Na₂SO₄, and concentrated under reduced pressure. The crude material was purified by silica gel chromatography using a gradient of 20-30% EtOAc in petroleum ether to afford tert-butyl 3-(((tert-butyldimethylsilyl)oxy)methyl)-4-hydroxypiperidine-1-carboxylate. MS (ESI) m/z: no ionization.

Step 3: tert-Butyl 3-(((tert-butyldimethylsilyl)oxy)methyl)-4-oxopiperidine-1-carboxylate. To a mixture of DMSO (6.57 mL, 93 mmol) and DCM (160 mL) cooled to −78° C. was added oxalyl chloride (8.11 mL, 93 mmol) under N₂. The reaction mixture was stirred at −78° C. for 30 min. Then, a solution of tert-butyl 3-(((tert-butyldimethylsilyl)oxy)methyl)-4-hydroxypiperidine-1-carboxylate (16 g, 46.3 mmol) in DCM (20 mL) was added dropwise at the same temperature. The reaction mixture was stirred for 30 min and quenched by adding TEA (32.3 mL, 232 mmol) dropwise. The resulting mixture was diluted with ice cold H₂O and extracted with DCM (2×50 mL). The combined organic extracts were washed with H₂O (100 mL), brine (100 mL), dried over Na₂SO₄, and concentrated under reduced pressure. The crude material was purified by silica gel chromatography, eluting with a gradient of 20-60% EtOAc in petroleum ether, to provide tert-butyl 3-(((tert-butyldimethylsilyl)oxy)methyl)-4-oxopiperidine-1-carboxylate. MS (ESI) m/z: 244.2 (M+H−Boc)⁺.

Step 4: tert-Butyl 4-(3-(benzyloxy)prop-1-yn-1-yl)-3-(((tert-butyldimethylsilyl)oxy)methyl)-4-hydroxypiperidine-1-carboxylate. To a solution of ((prop-2-yn-1-yloxy)methyl)benzene (2.55 g, 17.5 mmol) in THF (50 mL) at −78° C. was added n-BuLi (1.6 M in hexanes, 10.9 mL, 17.5 mmol) and stirred for 30 min at −78° C. tert-Butyl 3-(((tert-butyldimethylsilyl)oxy)methyl)-4-oxopiperidine-1-carboxylate (5.0 g, 14.6 mmol) was added dropwise and the reaction mixture was stirred for 2 h. The reaction mixture was quenched with sat. NH₄Cl solution and extracted with EtOAc (2×25 mL). The combined organic extracts were washed with brine (10 mL) and dried over Na₂SO₄, The solution was filtered and concentrated to provide tert-butyl 4-(3-(benzyloxy)prop-1-yn-1-yl)-3-(((tert-butyldimethylsilyl)oxy)methyl)-4-hydroxypiperidine-1-carboxylate that was carried forward to next step.

Step 5: tert-Butyl 3-(((tert-butyldimethylsilyl)oxy)methyl)-4-hydroxy-4-(3-hydroxypropyl)piperidine-1-carboxylate. To a solution of tert-butyl 4-(3-(benzyloxy)prop-1-yn-1-yl)-3-(((tert-butyldimethylsilyl)oxy)methyl)-4-hydroxypiperidine-1-carboxylate (2.0 g, 4.08 mmol) in a mixture of EtOH (20 mL), AcOH (4 mL), and H₂O (2 mL) purged with N₂, was added Pd/C (10% w/w, 1.0 g, 0.94 mmol), The mixture was allowed to stir under 14 psi of H₂ pressure for 16 h. The reaction mixture was filtered through a celite plug and the celite washed with 10% MeOH in DCM (50 mL). The filtrate was concentrated to provide tert-butyl 3-(((tert-butyldimethylsilyl)oxy)methyl)-4-hydroxy-4-(3-hydroxypropyl)piperidine-1-carboxylate. MS (ESI) m/z: 404.1 (M+H)⁺.

Step 6: tert-Butyl 3-(((tert-butyldimethylsilyl)oxy)methyl)-4-hydroxy-4-(3-((methylsulfonyl)oxy)propyl)piperidine-1-carboxylate. To a solution of tert-butyl 3-(((tert-butyldimethylsilyl)oxy)methyl)-4-hydroxy-4-(3-hydroxypropyl)piperidine-1-carboxylate (0.6 g, 1.49 mmol) in DCM (6 mL) at 0° C. was added DIPEA (0.52 mL, 2.97 mmol) followed by MsCl (0.13 mL, 1.64 mmol) and stirred at rt for 1 h. The reaction mixture was diluted with H₂O (100 mL) and extracted with DCM (100 mL). The organic extract was washed with brine and dried over Na₂SO₄. The solution was filtered and concentrated in vacuo to give tert-butyl 3-(((tert-butyldimethylsilyl)oxy)methyl)-4-hydroxy-4-(3-((methylsulfonyl)oxy)propyl)piperidine-1-carboxylate. The crude product was taken to the next step.

Step 7: tert-Butyl 6-(((tert-butyldimethylsilyl)oxy)methyl)-1-oxa-(8-azaspiro[4.5]decane-8-carboxylate. To a solution of tert-butyl 3-(((tert-butyldimethylsilyl)oxy)methyl)-4-hydroxy-4-(3-((methylsulfonyl)oxy)propyl)piperidine-1-carboxylate (0.7 g, 1.45 mmol) in DMF (5 mL) at 0° C. was added NaH (60% in mineral oil, 0.23 g, 5.81 mmol) and stirred at rt for 1h. The reaction mixture was diluted with ice cold H₂O (50 mL) and extracted with EtOAc (50 mL). The organic extract was washed with brine (50 mL) and dried over Na₂SO₄. The solution was filtered and concentrated in vacuo to give the crude material. The crude material was purified by silica gel chromatography, eluting with a gradient of 2% to 10% EtOAc in petroleum ether, to provide tert-butyl 6-(((tert-butyldimethylsilyl)oxy)methyl)-1-oxa-(8-azaspiro[4.5]decane-8-carboxylate. MS (ESI) m/z: 330.1 (M+H−56)⁺.

Step 8: 1-Oxa-8-azaspiro[4.5]decan-6-yl)methanol, Intermediate B-1. To a solution of tert-butyl 6-(((tert-butyldimethylsilyl)oxy)methyl)-1-oxa-8-azaspiro[4.5]decane-8-carboxylate (0.35 g, 0.91 mmol) in DCM (3 mL) at 0° C. was added TFA (0.70 mL, 9.08 mmol) dropwise and stirred at 25° C. for 2 b. The reaction mixture was concentrated in vacuo to give Intermediate B-1 as a TFA salt which was used without further purification. MS (ESI) m/z: 172.1 (M+H)⁺.

### (7R)-4,7-Dimethyl-1-oxa-8-azaspiro[4.5]dec-3-ene (Intermediate B-2)

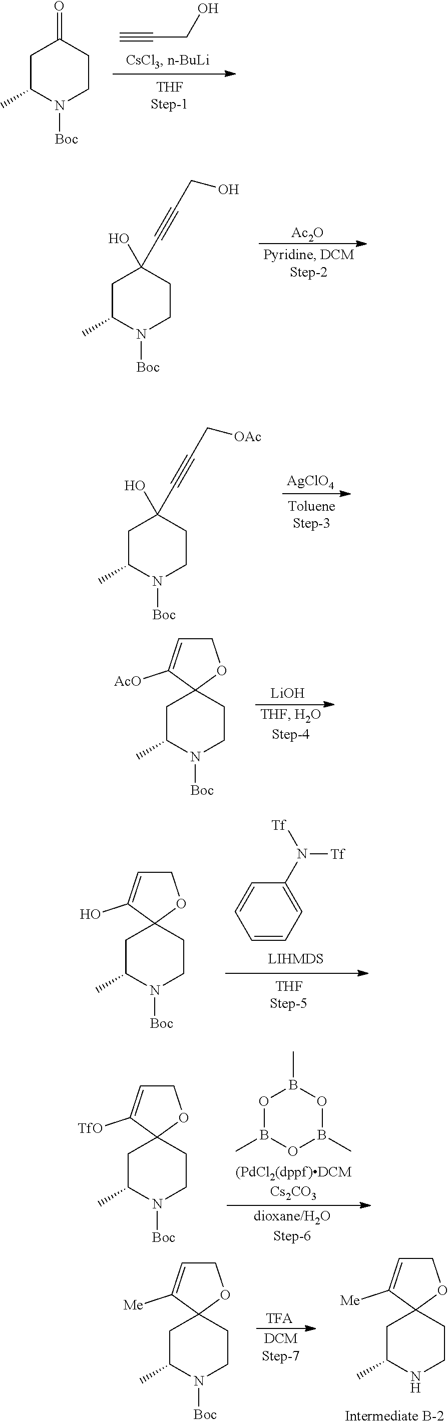

Step 1: tert-Butyl (2R)-4-hydroxy-4-(3-hydroxyprop-1-yn-1-yl)-2-methylpiperidine-1-carboxylate. To a stirred solution of propargyl alcohol (28.9 g, 516 mmol) in THF (300 mL) at −78° C. under N₂ atm, was added n-BuLi (2.5 M in hexanes, 248 mL, 619 mmol) and stirred for 40 min at −78° C., then 20 min at 0° C. The reaction mixture was again cooled to −78° C. and a pre-stirred solution of tert-butyl (R)-2-methyl-4-oxopiperidine-1-carboxylate (30 g, 141 mmol) and cerium (III) chloride (6.93 g, 28.1 mmol) in THF (150 mL) was added and stirred at −78° C. for 30 min. The resulting mixture was warmed to rt and stirred for 4 h. The reaction mixture was quenched with an aqueous HCl (1M, 200 mL) and extracted with EtOAc (2×250 mL). The combined organic extracts were washed with brine (20 mL), dried (Na₂SO₄), filtered, and concentrated under reduced pressure. The crude product was purified by silica gel chromatography, eluting with a gradient of 0% to 50% EtOAc/EtOH (3:1) in hexanes to give tert-butyl (2R)-4-hydroxy-4-(3-hydroxyprop-1-yn-1-yl)-2-methylpiperidine-1-carboxylate. MS (ESI) m/z: 170.1 (M−Boc+1)⁺. ¹H NMR (400 MHz, DMSO-d₆) δ 5.76 (s, 1H), 5.12 (t, J=5.9 Hz, 1H), 4.11-4.20 (m, 1H), 4.07 (d, J=5.9 Hz, 2H), 3.69 (ddd, J=13.3, 4.7, 2.3 Hz, 1H), 3.06 (td, J=13.1, 2.6 Hz, 1H), 1.74-1.82 (m, 3H), 1.57 (td, J=13.1, 4.7 Hz, 1H), 1.39 (s, 9H), 1.14-1.23 (m, 3H).

Step 2: tert-Butyl (2R)-4-(3-acetoxyprop-1-yn-1-yl)-4-hydroxy-2-methylpiperidine-1-carboxylate. To a stirred solution of tert-butyl(2R)-4-hydroxy-4-(3-hydroxyprop-1-yn-1-yl)-2-methylpiperidine-1-carboxylate (22.0 g, 82 mmol) and pyridine (19.8 mL, 245 mmol) in DCM (220 mL) at 0° C. was added acetic anhydride (10.8 mL, 114 mmol) and stirred at rt for 16 h. The reaction mixture was concentrated under reduced pressure, redissolved in EtOAc (150 mL), and washed with sat. aq NaHCO₃ solution (80 mL). The organic extract was dried (Na₂SO₄), filtered, and concentrated under reduced pressure. The crude material was purified by silica gel chromatography, eluting with a gradient of 0% to 40% EtOAc in hexanes to afford ter-butyl (2R)-4-(3-acetoxyprop-1-yn-1-yl)-4-hydroxy-2-methylpiperidine-1-carboxylate. MS (ESI) m/z: 212.1 (M−Boc+1)⁺. ¹H NMR (400 MHz, DMSO-d₆) δ 5.55 (s, 1H), 4.70 (s, 2H), 4.16 (tt, J=7.6, 5.7 Hz, 1H), 3.69 (dq, J=13.4, 2.2 Hz, 1H), 3.05 (td, J=13.1, 2.5 Hz, 1H), 2.04 (s, 3H), 1.67-1.86 (m, 3H), 1.58 (td, J=12.9, 4.5 Hz, 1H), 1.39 (s, 9H), 1.19 (d, J=7.0 Hz, 3H).

Step 3: tert-Butyl (7R)-4-acetoxy-7-methyl-1-oxa-8-azaspiro[4.5]dec-3-ene-8-carboxylate. To a stirred solution of tert-butyl (2R)-4-(3-acetoxyprop-1-yn-1-yl)-4-hydroxy-2-methylpiperidine-1-carboxylate (18 g, 57.8 mmol) in toluene (200 mL) under N₂ was added silver perchlorate (6.0 g. 28.9 mmol). The reaction vessel was covered with aluminum foil and stirred at 120° C. for 16 h. The reaction mixture was concentrated under reduced pressure. The crude material was purified by silica gel chromatography eluting with a gradient of 0% to 10% EtOAc in hexanes to afford tert-butyl (7R)-4-acetoxy-7-methyl-1-oxa-8-azaspiro[4.5]dec-3-ene-8-carboxylate. MS (ESI) m/z: 212.3 (M−Boc+1)⁺. 1H NMR (400 MHz, DMSO-d₆) δ 5.72-5.81 (m, 1H), 4.57 (t, J=1.8 Hz, 2H), 4.30 (s, 1H), 3.83 (d, J=13.0 Hz, 1H), 2.19 (s, 3H), 1.79 (dd, J=13.9, 6.7 Hz, 1H), 1.45-1.67 (m, 4H), 1.51 (s, 9H), 1.12-1.33 (m, 3H).

Step 4: tert-Butyl (7R)-4-hydroxy-7-methyl-1-oxa-8-azaspiro[4.5]dec-3-ene-8-carboxylate. To a stirred solution of tert-butyl (7R)-4-acetoxy-7-methyl-1-oxa-8-azaspiro[4.5]dec-3-ene-8-carboxylate (11 g, 35.3 mmol) in THF (75 mL) and H₂O (25 mL) was added LiOH·H₂O (2.17 g, 53.0 mmol) and stirred for 16 h at rt. The reaction mixture was quenched with brine (50 mL) and extracted with EtOAc (2×100 mL). The combined organic extracts were dried (Na₂SO₄), filtered, and concentrated under reduced pressure. The crude material was purified by silica gel chromatography, eluting with a gradient of 0% to 50% EtOAc in hexanes to afford tert-butyl (7R)-4-hydroxy-7-methyl-1-oxa-8-azaspiro[4.5]dec-3-ene-8-carboxylate. MS (ESI) m/z: 170.1 (M−Boc+1)⁺.

Step 5: tert-Butyl (7R)-7-methyl-4-(((trifluoromethyl)sulfonyl)oxy)-1-oxa-8-azaspiro[4.5]dec-3-ene-8-carboxylate. To a stirred solution of tert-butyl (7R)-4-hydroxy-7-methyl-1-oxa-8-azaspiro[4.5]dec-3-ene-8-carboxylate (6.0 g, 22.3 mmol) in dry THF (60 mL) at −78° C. was added LiHMDS (1M in THF, 29 mL, 29.0 mmol) dropwise and stirred for 1 h. A solution of N-(5-chloropyridin-2-yl)-1,1,1-trifluoro-N-((trifluoro methyl)sulfonyl)methanesulfonamide (11.37 g, 29.0 mmol) in THF (30 mL) was added at −78° C. and stirred for 3 h. The reaction mixture was quenched with sat. aq. NH₄Cl solution (150 mL) and extracted with EtOAc (2×250 mL). The combined organic extracts were washed with brine (100 mL), dried (Na₂SO₄), filtered, and concentrated under reduced pressure. The crude material was purified by silica gel chromatography, eluting with a gradient of 0% to 10% EtOAc in hexanes to afford tert-butyl (7R)-7-methyl-4-(((trifluoromethyl)sulfonyl)oxy)-1-oxa-8-azaspiro[4.5]dec-3-ene-8-carboxylate. MS (ESI) m/z: 302.1 (M−Boc+1)⁺. ¹H NMR (400 MHz, DMSO-d₆) δ 6.17 (t, J=1.9 Hz, 1H), 4.64 (t, J=1.7 Hz, 2H), 4.20-4.30 (m, 1H), 3.86 (d, J=13.4 Hz, 1H), 3.05 (s, 1H), 1.73 (dd, J=13.9, 6.6 Hz, 1H), 1.47-1.67 (m, 3H), 1.40 (s, 9H), 1.21 (d, J=7.0 Hz, 3H).

Step 6: tert-Butyl (7R)-4,7-dimethyl-1-oxa-8-azaspiro[4.5]dec-3-ene-8-carboxylate. To a solution of tert-butyl (7R)-7-methyl-4-(((trifluoromethyl)sulfonyl)oxy)-1-oxa-8-azaspiro[4.5]dec-3-ene-8-carboxylate (5.5 g, 13.7 mmol) in 1,4-dioxane (50 mL) and H₂O (25 mL) under N₂ were added Cs₂CO₃ (13.39 g, 41.1 mmol) and 2,4,6-trimethyl-1,3,5,2,4,6-trioxatriborinane (50% solution in THE, 27.5 mL, 54.8 mmol). The reaction mixture was degassed and purged with N₂ gas for 5 min. To this was added Pd(dppf)Cl₂. DCM (1.12 g, 1.370 mmol) and stirred at 100° C. for 16 h. The reaction mixture was quenched with H₂O (30 mL) and extracted with EtOAc (2×50 mL). The combined organic extracts were dried (Na₂SO₄), filtered, and concentrated under reduced pressure. The crude material was purified by silica gel chromatography, eluting with a gradient of 0-5% EtOAc in hexanes to afford tert-butyl (7R)-4,7-dimethyl-1-oxa-8-azaspiro[4.5]dec-3-ene-8-carboxylate. MS (ESI) m/z: 168.1 (M−Boc+1)⁺. ¹H NMR (400 MHz, DMSO-d₆) δ 5.56 (h, J=1.6 Hz, 1H), 4.42 (h, J=2.3 Hz, 2H), 3.82 (s, 1H), 3.06 (d, J=27.3 Hz, 1H), 1.79 (dd, J=13.6, 6.7 Hz, 1H), 1.80-1.60 (m, 5H), 1.40 (m, 11H), 1.22 (d, J=7.0 Hz, 3H).

Step 7: (7R)-4,7-Dimethyl-1-oxa-8-azaspiro[4.5]dec-3-ene (TFA salt), Intermediate B-2. To a solution of tert-butyl (7R)-4,7-dimethyl-1-oxa-8-azaspiro[4.5]dec-3-ene-8-carboxylate (1.5 g, 5.6 mmol) in DCM (16 mL) under N₂ was added TFA (2.16 mL, 28.1 mmol) at 0° C. and stirred at rt for 3 h. The reaction mixture was concentrated under reduced pressure to afford Intermediate B-2, as a TFA salt. MS (ESI) m/z: 168.1 (M+H)⁺. ¹H NMR (400 MHz, DMSO-d₆) δ 5.64 (q, J=1.6 Hz, 1H), 4.43 (h, J=2.2 Hz, 2H), 3.57-3.71 (m, 1H), 2.96-3.33 (m, 2H), 2.09 (s, 3H), 2.00 (d, J=5.6 Hz, 1H), 1.87-1.95 (m, 2H), 1.43-1.64 (m, 2H), 1.38 (d, J=7.0 Hz, 3H).

The below example was prepared in a manner similar to that described above.

TABLE 1-1

| | Chemical | | MS (ESI) |
|---|---|---|---|
| Int. No. | Structure | Name | m/z: (M + H)⁺ |
| B-3 | 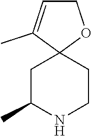 | (7S)-4.7-dimethyl-1- oxa-8-azaspiro[4.5]dec- 3-ene | 168.1 |

### 4H-Spiro[furo[3,4-d]thiazole-6,4′-piperidine] (Intermediate B-4)

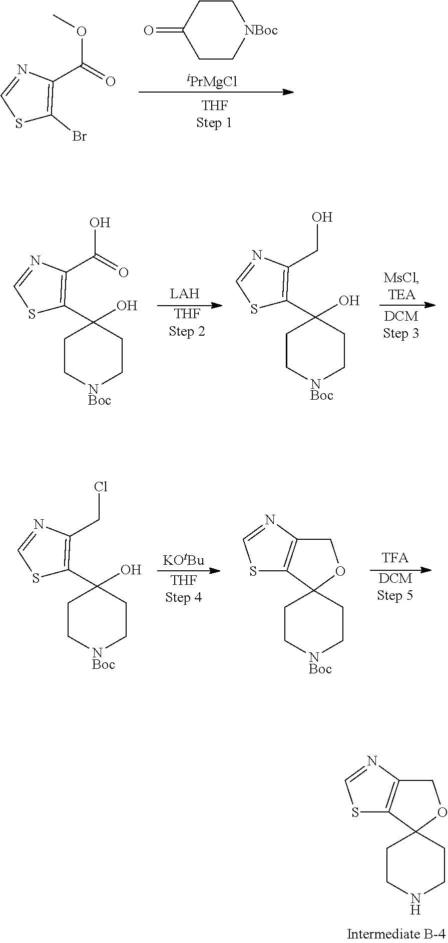

Step 1: 5-(1-(tert-Butoxycarbonyl)-4-hydroxypiperidin-4-yl) thiazole-4-carboxylic acid. To a solution of methyl 5-bromothiazole-4-carboxylate (15 g, 67.6 mmol) in THF (150 mL) was added isopropylmagnesium chloride (2M in THF, 50.7 mL, 101 mmol) dropwise at −70° C. After the addition, the mixture was stirred at −70° C. for 30 min, and then tert-butyl 4-oxopiperidine-1-carboxylate (26.9 g, 135 mmol) in THF (100 mL) was added dropwise at −70° C. The resulting mixture was stirred at 20° C. for 1 h. The reaction mixture was quenched by addition of 300 mL aq. NH₄Cl at 20° C. diluted with EtOAc (100 mL), and extracted with EtOAc (70 mL×3). The combined organic extracts were washed with brine (100 mL), dried over Na₂SO₄, filtered, and concentrated under reduced pressure to give 5-(1-(tert-butoxycarbonyl)-4-hydroxypiperidin-4-yl) thiazole-4-carboxylic acid.

Step 2: tert-Butyl 4-hydroxy-4-(4-(hydroxymethyl)thiazol-5-yl)piperidine-1-carboxylate. To a solution of 5-(1-(tert-butoxycarbonyl)-4-hydroxypiperidin-4-yl)thiazole-4-carboxylic acid (5.2 g, 15.8 mmol) in THF (100 mL) was added LAH (0.601 g, 15.8 mmol) in portions at 0° C. The reaction mixture was stirred at 20° C. for 12 h. The reaction mixture was quenched by addition of 200 mL H₂O at 10° C., and then diluted with EtOAc (200 mL), and extracted with EtOAc (100 mL×3). The combined organic extracts were washed with brine (200 mL), dried over Na₂SO₄, filtered, and concentrated under reduced pressure. The residue obtained was purified by silica gel chromatography, eluting with a gradient of 0-66% petroleum ether in EtOAc to give tert-butyl 4-hydroxy-4-(4-(hydroxymethyl)thiazol-5-yl)piperidine-1-carboxylate.

Step 3: tert-Butyl 4-(4-(chloromethyl)thiazol-5-yl)-4-hydroxypiperidine-1-carboxylate. To a solution of tert-butyl 4-hydroxy-4-(4-(hydroxymethyl)thiazol-5-yl)piperidine-1-carboxylate (2.1 g, 6.7 mmol) in DCM (25 mL) was added TEA (1.86 mL, 13.4 mmol) at 0° C. Next, MsCl (1.15 g, 10.02 mmol) was added dropwise at 0° C. The resulting mixture was stirred at 20° C. for 3 h. The mixture was quenched with H₂O (80 mL), extracted with DCM (3×20 mL). The combined organic extracts were washed with brine (30 mL), dried over Na₂SO₄, filtered, and concentrated to give tert-butyl 4-(4-(chloromethyl)thiazol-5-yl)-4-hydroxypiperidine-1-carboxylate.

Step 4: tert-Butyl 4H-spiro[furo[3,4-d]thiazole-6,4′-piperidine]-1′-carboxylate. To a solution of terf-butyl 4-(4-(chloromethyl)thiazol-5-yl)-4-hydroxypiperidine-1-carboxylate (3 g, 9.0 mmol) in THF (10 mL) was added KOtBu (2.02 g, 18.03 mmol). The mixture was stirred at 50° C. for 2 h and quenched by the addition of satd NH₄Cl. The reaction mixture was extracted with EtOAc (50 mL). The organic layer was washed with brine (200 mL), dried over Na₂SO₄, filtered and concentrated under reduced pressure to give a residue. The residue was purified by silica gel chromatography, eluting with a gradient of 50% to 75% petroleum ether in EtOAc to give tert-butyl 4H-spiro[furo[3,4-d]thiazole-6,4′-piperidine]-1′-carboxylate.

Step 5: 4H-Spiro[furo[3,4-d]thiazole-6,4′-piperidine], Intermediate B-4. To a solution of tert-butyl 4H-spiro[furo[3,4-d]thiazole-6,4′-piperidine]-1′-carboxylate (350 mg, 1.18 mmol) in DCM (5 mL) was added TFA (0.5 mL, 6.5 mmol). Then the mixture was stirred at 20° C. for 6 h. The reaction mixture was concentrated under reduced pressure and the residue was purified by silica gel chromatography, eluting with a gradient of 10-100% (0.1% NH₃·H₂O) MeOH in EtOAc to give Intermediate B-4. MS (ESI) m/z: 197.1 (M+H)⁺.

### tert-Butyl 4H-spiro[furo[3,4-d]thiazole-6,4′-piperidine]-1′-carboxylate (Intermediate B-5)

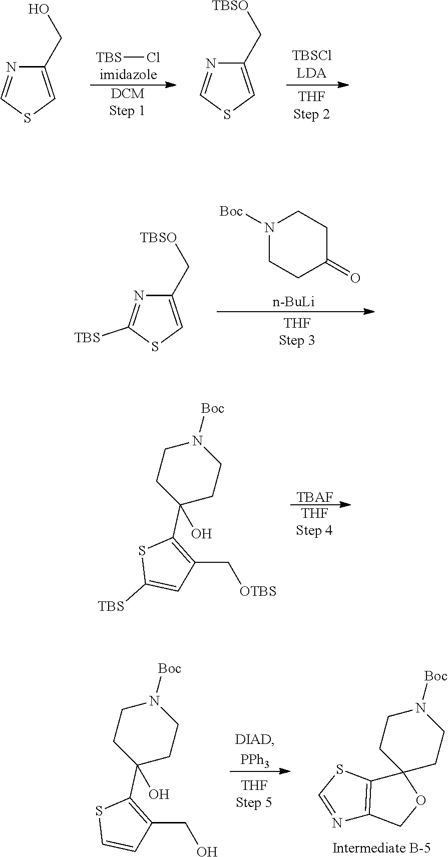

Step 1: 4-(((tert-Butyldimethylsilyl)oxy)methy)thiazole. To a glass vial was added thiazole-4-methanol (1.72 g, 15.0 mmol), DCM (30 mL), imidazole (2.04 g, 29.9 mmol), and TBS-Cl (2.48 g, 16.5 mmol). The reaction mixture was stirred at rt for 1 h. The reaction mixture was quenched with H₂O (20 mL) and the organic phase was separated. This was washed with H₂O (2×20 mL) and the combined aqueous phases were extracted with EtOAc (15 mL). The combined organic phases were washed with brine (15 mL), dried over Na₂SO₄, and concentrated. The crude material was purified by silica gel chromatography, eluting with a gradient of 0% to 25% EtOAc in heptane to give 4-(((tert-butyldimethylsilyl)oxy)methyl)thiazole. MS (ESI) m/z: 230.2 (M+H)⁺. ¹H NMR (400 MHz, CDCl₃) δ 8.76 (d, J=2.1 Hz, 1H), 7.25 (s, 1H), 4.94 (d, J=1.3 Hz, 2H), 0.97 (s, 9H), 0.14 (s, 6H).

Step 2:2-(tert-Butyldimethylsilyl)-4-(((tert-butyldimethylsilyl)oxy)methyl)thiazole. A solution of 4-(((tert-butyldimethylsilyl)oxy)methyl)thiazole (360 mg, 1.57 mmol) in THF (2 mL) at 0° C. was treated with LDA (1 M solution in THF, 2.04 mL, 2.04 mmol). After stirring at 0° C. for 1 h, a solution of (1,1-dimethylethyl)dimethylsilyl chloride (355 mg, 2.35 mmol) in THF (1.5 mL) was slowly added to the reaction mixture. The resulting mixture was stirred at rt for 0.5 h, quenched, and concentrated. The crude material was purified by silica gel chromatography, eluting with a gradient of 0% to 15% EtOAc in heptane to provide 2-(tert-butyldimethylsilyl)-4-(((tert-butyldimethylsilyl)oxy)methyl)thiazole. MS (ESI) m/z: 344.2 (M+H)⁺. ¹H NMR (400 MHz, CDCl₃) δ 7.35-7.42 (m, 1H), 4.98-5.07 (m, 2H), 0.99 (s, 9H), 0.97 (s, 9H), 0.38-0.40 (m, 6H), 0.14 (s, 6H).

Step 3: tert-Butyl 4-(2-(ter-butyldimethylsilyl)-4-(((tert-butyldimethylsilyl)oxy)methyl)thiazol-5-yl)-4-hydroxypiperidine-1-carboxylate. To a solution of 2-(tert-butyldimethylsilyl)-4-(((tert-butyldimethylsilyl)oxy)methyl)thiazole (450 mg, 1.31 mmol) in THF (6.6 mL) at −78° C. was slowly added n-BuLi (1.6 M in hexanes, 524 μL, 1.31 mmol). After addition, the reaction mixture was warmed to 0° C. and stirred at 0° C. for 40 min. The above reaction mixture was added to a solution of 1-tert-butoxycarbonylpiperidin-4-one (274 mg, 1.38 mmol) in 2 mL THF at 0° C. The resulting reaction mixture was warmed to rt and stirred for 44 h. The reaction mixture was quenched by addition of sat. aq. NH₄Cl (6 mL), extracted with EtOAc (3×6 mL), washed with brine, dried through Na₂SO₄, and concentrated. The crude material was purified by silica gel chromatography, eluting with a gradient of 0% to 80% EtOAc in heptane to provide two products. The first eluting peak was identified as tert-butyl 4-(2-(tert-butyldimethylsilyl)-4-(((tert-butyldimethylsilyl)oxy)methyl)thiazol-5-yl)-4-hydroxypiperidine-1-carboxylate. MS (ESI) m/z: 543.2 (M+H)⁺. ¹H NMR (400 MHz, CDCl₃) δ 5.15 (s, 2H), 3.98 (br s, 2H), 3.28 (br t, J=12.1 Hz, 2H), 2.04 (br d, J=10.9 Hz, 2H), 1.88-1.98 (m, 2H), 1.38-1.60 (m, 9H), 0.93-1.01 (m, 18H), 0.26-0.46 (m, 6H), 0.07-0.24 (m, 6H). The second eluting peak was identified as tert-butyl 4-(2-(tert-butyldimethylsilyl)-4-(hydroxymethyl)thiazol-5-yl)-4-hydroxypiperidine-1-carboxylate. MS (ESI) m/z: 429.2 (M+H)⁻. ¹H NMR (400 MHz, CDCl₃) δ 4.65-4.74 (m, 2H), 3.93-4.09 (m, 2H), 3.38-3.48 (m, 1H), 3.22-3.36 (m, 2H), 2.05-2.15 (m, 2H), 1.84-1.92 (m, 2H), 1.48-1.50 (m, 9H), 0.92-0.95 (m, 9H), 0.34-0.38 (m, 6H).

Step 4: tert-Butyl 4-hydroxy-4-(4-(hydroxymethyl)thiazol-5-yl)piperidine-1-carboxylate. To a glass vial with a stir bar was added tetrabutylammonium fluoride hydrate (111 mg, 0.40 mmol), tert-butyl 4-(2-(tert-butyldimethylsilyl)-4-(((ter-butyldimethylsilyl)oxy)methyl)thiazol-5-yl)-4-hydroxypiperidine-1-carboxylate (72 mg, 0.13 mmol), and THE (1.3 mL). The mixture was stirred at rt for 2 h. The reaction mixture was quenched by aq. sat. NH₄Cl (3 mL), extracted with EtOAc (3×3 mL). The combined organic extracts were washed with brine, dried through Na₂SO₄, and concentrated. The crude material was purified by silica gel chromatography, eluting with a gradient of 0% to 8% MeOH in DCM, to provide tert-butyl 4-hydroxy-4-(4-(hydroxymethyl)thiazol-5-yl)piperidine-1-carboxylate. MS (ESI) m/z: 315.2 (M+H)⁺. ¹H NMR (400 MHz, CDCl₃) δ 8.55 (s, 1H), 4.96 (s, 2H), 4.00 (br d, J=11.5 Hz, 2H), 3.12-3.36 (m, 2H), 2.00-2.07 (m, 2H), 1.87-1.98 (m, 2H), 1.40-1.56 (m, 9H).

Step 5: tert-Butyl 4H-spiro[furo[3,4-d]thiazole-6,4′-piperidine]-1′-carboxylate, Intermediate B-5. To a glass vial with a stir bar was added DIAD (21 μL, 0.11 mmol) in 0.8 mL THF, and triphenylphosphine (28 mg, 0.11 mmol) in 0.8 mL THF was added at 0° C. The reaction mixture was stirred for 10 min, and was allowed to warm to rt. A solution of tert-butyl 4-hydroxy-4-(4-(hydroxymethyl)thiazol-5-yl)piperidine-1-carboxylate (17 mg, 0.054 mmol) in THF (1 mL) was added to the reaction mixture. The resulting mixture was stirred at rt for 13 h. The reaction mixture was quenched by sat. aq. NH₄Cl (3 mL), extracted with EtOAc (3×2 mL). The combined organic extracts were washed with brine, dried over Na₂SO₄, and concentrated. The crude material was purified by silica gel chromatography, eluting with a gradient of 3-80% EtOAc in heptane, to give Intermediate B-5. MS (ESI) m/z: 297.2 (M+H)⁺. ¹H NMR (400 MHz, CDCl₃) δ 8.70-8.83 (m, 1H), 5.08 (s, 2H), 3.61 (br t, J=5.4 Hz, 4H), 1.82-1.94 (m, 4H), 1.50 (d, J=1.0 Hz, 9H).

### 5-Methyl-1-oxa-9-azaspiro[5.5]undec-4-ene (Intermediate B-6)

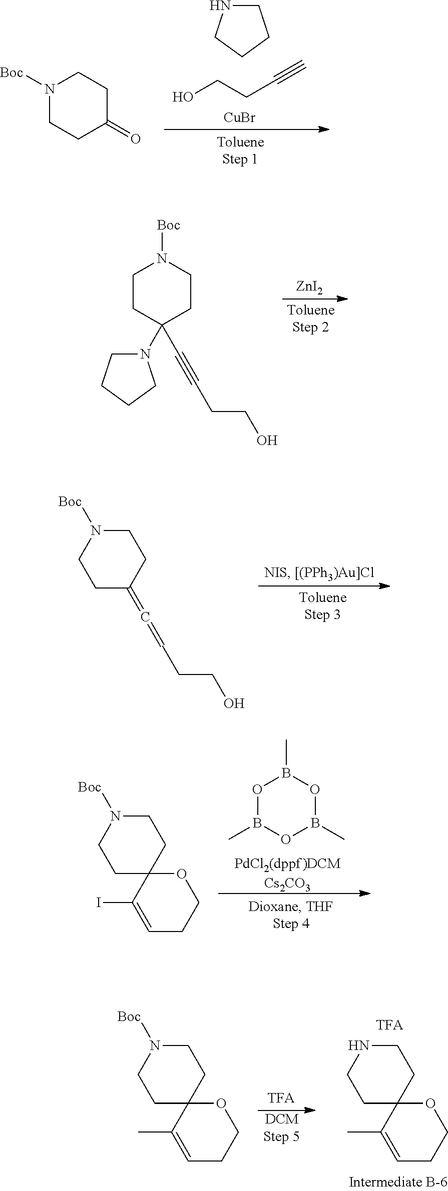

Step 1: tert-Butyl 4-(4-hydroxybut-1-yn-1-yl)-4-(pyrrolidin-1-yl)piperidine-1-carboxylate. To a stirred solution of but-3-yn-1-ol (823 mg, 11.7 mmol) in toluene (20 mL) under N₂ was added pyrrolidine (0.913 mL, 11.0 mmol), copper(I) bromide (216 mg, 1.51 mmol), and tert-butyl 4-oxopiperidine-1-carboxylate (2 g. 10.0 mmol) at rt. The reaction mixture was stirred at 100° C. for 10 h. The reaction mixture was filtered through a pad of celite plug and the plug was washed it with EtOH. The filtrate was concentrated in vacuo. The crude material was purified by silica gel chromatography, eluting with a gradient of 0% to 20% EtOAc in hexanes, to provide tert-butyl 4-(4-hydroxybut-1-yn-1-yl)-4-(pyrrolidin-1-yl)piperidine-1-carboxylate. MS (ESI) m/z: 323.3 (M+H)⁺.

Step 2: tert-Butyl 4-(4-hydroxybut-1-en-1-ylidene)piperidine-1-carboxylate. A solution of tert-butyl 4-(4-hydroxybut-1-yn-1-yl)-4-(pyrrolidin-1-yl)piperidine-1-carboxylate (15 g, 46.5 mmol) in toluene (150 mL) was stirred at rt under N₂ and zinc iodide (8.91 g, 27.9 mmol) was added at rt. The reaction mixture was stirred at 120° C. for 6 h and filtered through a celite plug, and the plug was washed with EtOH. The filtrate was concentrated in vacuo and the crude material was purified by silica gel chromatography, eluting with a gradient of 0% to 20% EtOAc in hexanes, to provide tert-butyl 4-(4-hydroxybut-1-en-1-ylidene) piperidine-1-carboxylate.

Step 3: tert-Butyl 5-iodo-1-oxa-9-azaspiro[5.5]undec-4-ene-9-carboxylate. A solution of tert-butyl 4-(4-hydroxybut-1-en-1-ylidene) piperidine-1-carboxylate (3 g, 11.84 mmol) in toluene (10 mL) was stirred at rt under N₂ and [(PPh₃)Au]Cl (5.86 g, 11.84 mmol) and NIS (4 g, 17.76 mmol) were added at rt. The reaction mixture was stirred at 120° C. for 6 h and cooled to rt. The reaction mixture was filtered through a pad of celite, and the celite was washed with EtOH. The filtrate was concentrated in vacuo to give the crude material, which was absorbed onto a plug of silica gel and purified by chromatography, eluting with a gradient of 0% to 20% EtOAc in hexanes, to provide tert-butyl 5-iodo-1-oxa-9-azaspiro[5.5]undec-4-ene-9-carboxylate.

Step 4: tert-Butyl 5-methyl-1-oxa-9-azaspiro[5.5]undec-4-ene-9-carboxylate. To solution of tert-butyl 5-iodo-1-oxa-9-azaspiro[5.5]undec-4-ene-9-carboxylate (1.1 g, 2.90 mmol) in 1,4-dioxane (18 mL) and H₂O (6 mL) under N₂ at rt, Cs₂CO₃ (2.84 g, 8.70 mmol) and 2,4,6-trimethyl-1,3,5,2,4,6-trioxatriborinane (50% in THF) (2.91 g. 11.60 mmol) were added. The reaction mixture was purged with N₂ for 5 min, and then PdCl₂(dppf). DCM (237 mg, 0.290 mmol) was added. The resulting mixture was heated to 100° C. and stirred for 16 h. The reaction mixture was quenched with H₂O (30 mL) and extracted with EtOAc (2×50 mL). The combined organic extracts were dried over Na₂SO₄, filtered, and concentrated in vacuo. The crude material was purified by silica gel chromatography, eluting with a gradient of 0-5% EtOAc in petroleum ether, to provide tert-butyl 5-methyl-1-oxa-9-azaspiro[5.5]undec-4-ene-9-carboxylate. MS (ESI) m/z: 168.1 (M−Boc+H)¹.

Step 5: 5-Methyl-1-oxa-9-azaspiro[5.5]undec-4-ene (TFA salt), Intermediate B-6. To solution of tert-butyl 5-methyl-1-oxa-9-azaspiro[5.5]undec-4-ene-9-carboxylate (540 mg, 2.02 mmol) in DCM (16 mL) under N₂ at 0° C. was added TFA (0.78 mL, 10.1 mmol). The reaction mixture was stirred at rt for 3 h and concentrated in vacuo to give Intermediate B-6, as a TFA salt. MS (ESI) m/z: 168.1(M+H)⁺.

### 3′-Methyl-6′,7′-dihydrospiro[piperidine-4,4′-pyrazolo[5,1-c][1,4]oxazine] (Intermediate B-7)

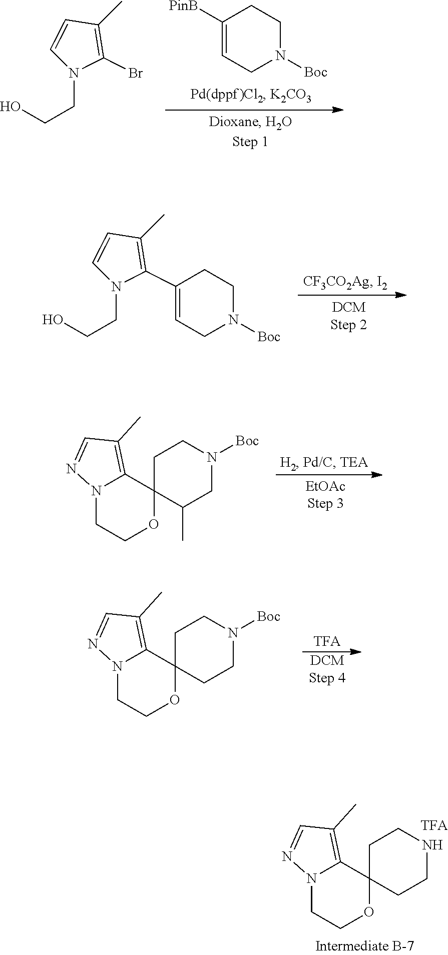

Step 1: tert-Butyl 4-(1-(2-hydroxyethyl)-4-methyl-1H-pyrazol-5-yl)-3,6-dihydropyridine-1(2H)-carboxylate. To a solution of 2-(5-bromo-4-methyl-1H-pyrazol-1-yl) ethan-1-ol (3 g, 14.6 mmol) in 1,4-dioxane (60 mL) and H₂O (12 mL) was added tert-butyl 4-(4,4,5,5-tetramethyl-1,3,2-dioxaborolan-2-yl)-3,6-dihydropyridine-1(2H)-carboxylate (5.43 g, 17.56 mmol), K₂CO₃ (6.07 g, 43.9 mmol), and Pd(dppf)Cl₂ (535 mg, 0.732 mmol) in sequence. The mixture was stirred at 90° C. for 3 h. The reaction mixture was filtered, the filter cake was washed with EtOAc. and the filtrate was concentrated under reduced pressure. The residue was purified by silica gel chromatography, eluting with a gradient of 33% to 50% EtOAc in petroleum ether to give tert-butyl 4-(1-(2-hydroxyethyl)-4-methyl-1H-pyrazol-5-yl)-3,6-dihydropyridine-1 (2H)-carboxylate. MS (ESI) m/z: 308.3 (M+H)⁺.

Step 2: tert-Butyl 3-iodo-3′-methyl-6′,7′-dihydrospiro[piperidine-4,4′-pyrazolo[5,1-c][1,4]oxazine]-1-carboxylate. To a solution of tert-butyl 4-(1-(2-hydroxyethyl)-4-methyl-1H-pyrazol-5-yl)-3,6-dihydropyridine-1 (2H)-carboxylate (2 g, 6.51 mmol) in DCM (50 mL) was added 12 (8.26 g, 32.5 mmol) and CF₃CO₂Ag (2.87 g, 13.01 mmol). The mixture was stirred at 20° C. for 3 h. The reaction mixture was filtered, washed with EtOAc, and concentrated under reduced pressure. The residue was purified by silica gel chromatography, eluting with a gradient of 20% to 33% EtOAc in petroleum ether/to give tert-butyl 3-iodo-3′-methyl-6′,7-dihydrospiro[piperidine-4,4′-pyrazolo[5,1-c][1,4]oxazine]-1-carboxylate.

Step 3: tert-Butyl 3′-methyl-6′,7′-dihydrospiro[piperidine-4,4′-pyrazolo[5,1-c][1.4]oxazine]-1-carboxylate. To a suspension of Pd/C (10% w/w, 900 mg, 1.04 mmol) in EtOAc (40 mL) was added TEA (434 μL, 3.12 mmol) and tert-butyl 3-iodo-3′-methyl-6′,7′-dihydrospiro[piperidine-4,4′-pyrazolo[5,1-c][1,4]oxazine]-1-carboxylate (450 mg, 1.04 mmol) under Ar atmosphere. The suspension was degassed and purged 3 times with H₂. The mixture was stirred under H₂ (50 psi) at 20° C. for 12 h. The reaction mixture was filtered, washed with EtOAc, and concentrated under reduced pressure. The residue was purified by silica gel chromatography, eluting with a gradient of 0% to 33% EtOAc in petroleum ether to give tert-butyl 3′-methyl-6′,7′-dihydrospiro[piperidine-4,4′-pyrazolo[5,1-c][1,4]oxazine]-1-carboxylate. MS (ESI) m/z: 308.2 (M+H)⁺.

Step 4: 3′-Methyl-6′,7′-dihydrospiro[piperidine-4,4′-pyrazolo[5,1-c][1,4]oxazine] (TFA salt). To a solution of tert-butyl 3′-methyl-6′,7′-dihydrospiro[piperidine-4,4′-pyrazolo[5,1-c][1,4]oxazine]-1-carboxylate (250 mg, 0.81 mmol) in DCM (5 mL) was added TFA (1 mL). The mixture was stirred at 20° C. for 1 h under N₂ atmosphere. The reaction mixture was concentrated under reduced pressure to obtain Intermediate B-7, as a TFA salt. MS (ESI) m/z: 208.2 (M+H)⁺.

The intermediate in the table below was prepared in a fashion similar to that described above.

TABLE 1-2

| | | | MS (ESI) |
|---|---|---|---|
| Int. No. | Chemical Structure | Name | m/z: (M + H)⁺ |
| B-18 | 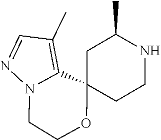 [SMILES: Cc1cnn2c1[C@]1(CCN[C@H](C)C1)OCC2] | (2R,4R)-2,3′-dimethyl-6′,7′- dihydrospiro[piperidine-4,4′- pyrazolo[5.1-c][1,4]oxazine] | 22.2.2 |

### 6′,7-Dihydrospiro[piperidine-4,4′-pyrano[4,3-d]thiazole] (Intermediate B-8)

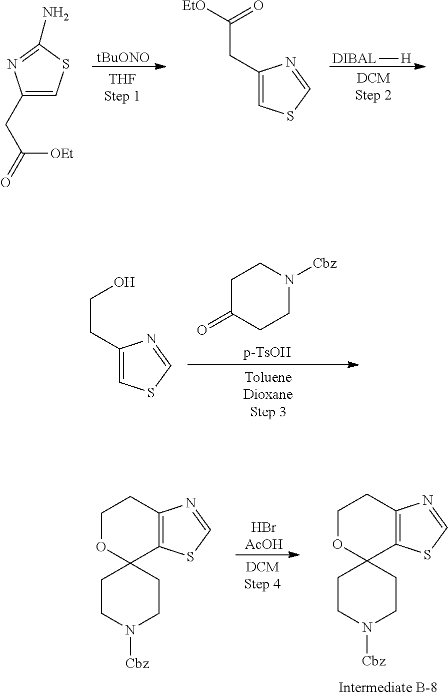

Step 1: Ethyl 2-(thiazol-4-yl)acetate. To a solution of ethyl 2-(2-aminothiazol-4-yl)acetate (50 g, 268 mmol) in THF (500 mL) was added tert-butyl nitrite (41.5 g, 403 mmol) at 25° C. The reaction mixture was stirred at 25° C. for 12 h. The solution was diluted with diethyl ether (10 mL) and washed with 25 mL of aq. 10% HCl solution. The aqueous phase was extracted with EtOAc (20 mL) and the combined organic phases were concentrated under reduced pressure. The residue was purified by silica gel chromatography, eluting with a gradient of 10% to 33% EtOAc in petroleum ether to give ethyl 2-(thiazol-4-yl)acetate.

Step 2:2-(Thiazol-4-yl) ethan-1-ol. To a stirred solution of ethyl 2-(thiazol-4-yl)acetate (18 g, 105 mmol) in DCM (400 mL) was added DIBAL-H (1 M in THF, 315 mL, 315 mmol) at 0° C. The reaction mixture was stirred at 25° C. under N₂ for 3 h. The reaction was quenched with aq. sat. NaHCO₃ (500 mL), extracted with DCM (500 mL×3) and the combined organic extracts were washed with brine (1000 mL), dried over Na₂SO₄, and concentrated under reduced pressure to give 2-(thiazol-4-yl) ethan-1-ol.

Step 3: Benzyl 6′,7′-dihydrospiro[piperidine-4,4′-pyrano[4,3-d]thiazole]-1-carboxylate. To a solution of 2-(thiazol-4-yl) ethan-1-ol (0.2 g. 1.5 mmol) in toluene (8 mL) and 1,4-dioxane (2 mL) was added benzyl 4-oxopiperidine-1-carboxylate (542 mg, 2.32 mmol) and p-TsOH (267 mg, 1.55 mmol). The mixture was stirred at 120° C. for 12 h. The reaction mixture was adjusted to pH=7~8 with sat. NaHCO₃, and then concentrated under reduced pressure. The residue was purified by silica gel chromatography, eluting with a gradient of 20% to 50% EtOAc in petroleum ether to provide benzyl 6′,7′-dihydrospiro[piperidine-4,4′-pyrano[4,3-d]thiazole]-1-carboxylate. MS (ESI) m/z: 345.3 (M+H)⁺.

Step 4: 6′,7-Dihydrospiro[piperidine-4,4′-pyrano[4,3-d]thiazole], Intermediate B-8. To a solution of benzyl 6′,7′-dihydrospiro[piperidine-4,4′-pyrano[4,3-d]thiazole]-1-carboxylate (970 mg, 2.82 mmol) in DCM (2 mL) was added HBr in AcOH (33%, 4.18 g, 9.86 mmol) and the mixture was stirred at 20° C. for 5 h. The reaction mixture was adjusted to pH=7~8 with NH₃H₂O and concentrated under reduced pressure. The residue was purified by silica gel chromatography, eluting with a gradient of 3% to 100% MeOH (0.1% NH₃·H₂O) in EtOAc to provide Intermediate B-8. MS (ESI) m/z: 211.1 (M+H)⁺.

### tert-Butyl-2-methyl-4-oxo-1-oxa-8-azaspiro[4.5]decane-8-carboxylate (Intermediate B-9)

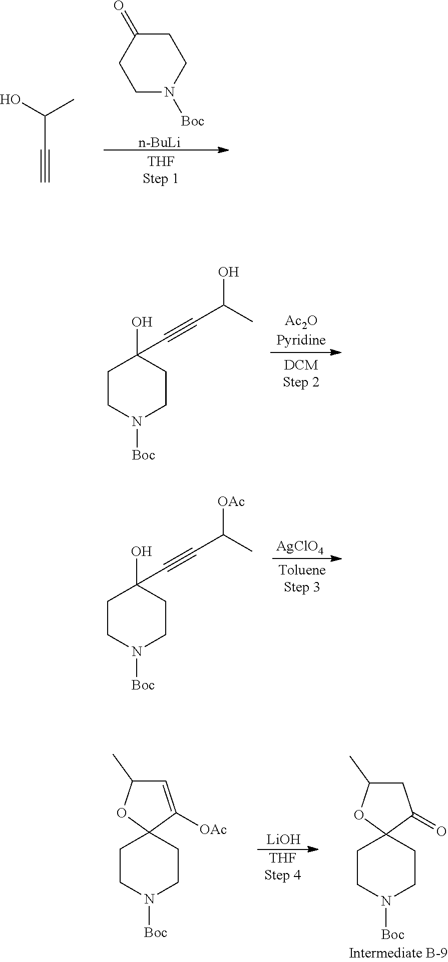

Step 1: tert-Butyl 4-hydroxy-4-(3-hydroxybut-1-ynyl)piperidine-1-carboxylate. To a solution of but-3-yn-2-ol (25 g. 357 mmol) in THF (1000 mL) was added dropwise n-BuLi (2.5 M in hexanes, 313.9 mL, 785 mmol) at −78° C. and stirred for 1 h. tert-Butyl 4-oxopiperidine-1-carboxylate (71.1 g, 357 mmol) was added to the mixture and was stirred at 25° C. for 11 h. The reaction mixture was quenched by addition of H₂O (1000 mL) at 0° C., and then extracted with EtOAc (500 mL×2). The combined organic extracts were dried over Na₂SO₄, filtered, and concentrated under reduced pressure. The residue was purified by silica gel chromatography, eluting with a gradient of 10% to 50% EtOAc in petroleum ether to provide tert-butyl 4-hydroxy-4-(3-hydroxybut-1-ynyl)piperidine-1-carboxylate. ¹H NMR (400 MHz, CDCl₃) δ 4.61-4.55 (m, 1H), 3.78-3.74 (m, 2H), 3.25 (t, J=10.4 Hz, 2H), 2.40-2.20 (m, 2H), 1.95-1.80 (m, 2H), 1.75-1.65 (m, 2H), 1.46 (s, 12H).

Step 2: tert-Butyl 4-(3-acetoxybut-1-ynyl)-4-hydroxy-piperidine-1-carboxylate. To a solution of tert-butyl 4-hydroxy-4-(3-hydroxybut-1-ynyl)piperidine-1-carboxylate (22 g, 81.7 mmol) in DCM (150 mL) was added pyridine (3.96 mL, 49.0 mmol) and Ac₂O (110.0 mL, 1.2 mol). The mixture was stirred at 25° C. for 5 h. The mixture was concentrated under reduced pressure and the residue was purified by silica gel chromatography, eluting with 1% to 50% EtOAc in petroleum ether to provide tert-butyl 4-(3-acetoxybut-1-ynyl)-4-hydroxy-piperidine-1-carboxylate.

Step 3: tert-Butyl 4-acetoxy-2-methyl-1-oxa-8-azaspiro[4.5]dec-3-ene-8-carboxylate. To a solution of tert-butyl 4-(3-acetoxy but-1-ynyl)-4-hydroxy-piperidine-1-carboxylate (19 g, 61 mmol) in toluene (200 mL) was added AgClO₄ (1.39 g, 6.71 mmol). The reaction was stirred at 80° C. for 19 h. The mixture was diluted with DCM (500 mL) and washed with 10% aq. NH₄OH (300 mL). The organic layer was dried over Na₂SO₄, filtered, and concentrated under reduced pressure. The residue was purified by silica gel chromatography, eluting with a gradient of 3% to 33% EtOAc in petroleum ether to provide tert-butyl 4-acetoxy-2-methyl-1-oxa-8-azaspiro[4.5]dec-3-ene-8-carboxylate.

Step 4: tert-Butyl 2-methyl-4-oxo-1-oxa-8-azaspiro[4.5]decane-8-carboxylate, Intermediate B-9. To a solution of tert-butyl 4-acetoxy-2-methyl-1-oxa-8-azaspiro[4.5]dec-3-ene-8-carboxylate (27.5 g, 88.3 mmol) in THF (300 mL) was added LiOH·H₂O (5.96 g, 142 mmol) in H₂O (50 mL). The mixture was stirred at 25° C. for 3 h. The reaction mixture was diluted with H₂O (100 mL) and extracted with EtOAc (100 mL×2). The combined organic extracts were dried over Na₂SO₄, filtered, and concentrated under reduced pressure. The residue was purified by silica gel chromatography, eluting with a gradient of 1% to 10% EtOAc in petroleum ether to provide Intermediate B-9. MS (ESI) m/z: 170.1 (M-Boc+H)⁺.

### tert-Butyl 4-methyl-1-oxa-8-azaspiro[4.5]dec-3-ene-8-carboxylate (Intermediate B-10)

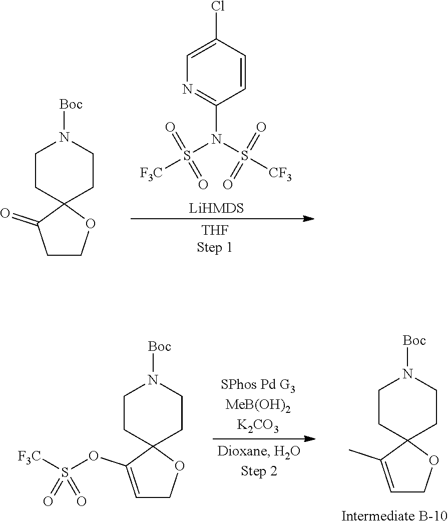 [SMILES: CC(C)(C)OC(=O)N1CCC2(CC1)OCCC2=O ; O=S(=O)(N(c1ccc(Cl)cn1)S(=O)(=O)C(F)(F)F)C(F)(F)F ; CC(C)(C)OC(=O)N1CCC2(CC1)OCC=C2OS(=O)(=O)C(F)(F)F ; CC1=CCOC12CCN(C(=O)OC(C)(C)C)CC2]

Step 1: ter-Butyl 4-(((trifluoromethyl)sulfonyl)oxy)-1-oxa-8-azaspiro[4.5]dec-3-ene-8-carboxylate. To a −78° C. solution of tert-butyl 4-oxo-1-oxa-8-azaspiro[4.5]decane-8-carboxylate (1 g, 3.9 mmol) in dry THF (10 mL) was added LiHMDS (1 M in THF, 5.09 mL, 5.09 mmol) was added dropwise at −78° C., and the reaction mixture stirred at −78° C. for 1 h. To the reaction mixture, a solution of N-(5-chloropyridin-2-yl)-1,1,1-trifluoro-N((trifluoromethyl)sulfonyl)methanesulfonamide (2.0 g, 5.09 mmol) in THF (5 mL) was added and the reaction mixture was stirred at rt for 1 h. The reaction mixture was quenched with H₂O (10 mL) and extracted with EtOAc (2×25 mL). The organic extracts were washed with brine (10 mL), dried over Na₂SO₄, filtered, and concentrated under reduced pressure. The crude residue was purified by silica gel chromatography, eluting with a gradient of 5% to 10% EtOAc in petroleum ether to give tert-butyl 4-(((trifluoromethyl)sulfonyl)oxy)-1-oxa-8-azaspiro[4.5]dec-3-ene-8-carboxylate.

Step 2: tert-Butyl 4-methyl-1-oxa-8-azaspiro[4.5]dec-3-ene-8-carboxylate, Intermediate B-10. To a solution of tert-butyl 4-(((trifluoromethyl)sulfonyl)oxy)-1-oxa-8-azaspiro[4.5]dec-3-ene-8-carboxylate (450 mg, 1.2 mmol) in 1,4-dioxane (5 mL) and H₂O (1 mL) at rt under N₂ atm, was added K₂CO₃ (321 mg, 2.32 mmol) and methylboronic acid (278 mg, 4.65 mmol). The reaction mixture was purged by N₂ for 5 min, then SPhos Pd G3 (9.06 mg, 0.012 mmol) was added. The reaction mixture was heated to 100° C. and stirred for 16 h. The reaction mixture was quenched with H₂O (10 mL) and extracted with EtOAc (2×20 mL). The organic extracts were dried over Na₂SO₄, filtered, and concentrated in vacuo. The crude material was purified by silica gel chromatography, eluting with a gradient of 0% to 10% EtOAc in petroleum ether to provide Intermediate B-10. m/z (ESI): 212.1 (M−56+H)⁺

### tert-Butyl 1-methyl-1,4-dihydrospiro[furo[3,4-c]pyrazole-6,4′-piperidine]-1′-carboxylate (Intermediate B-11)

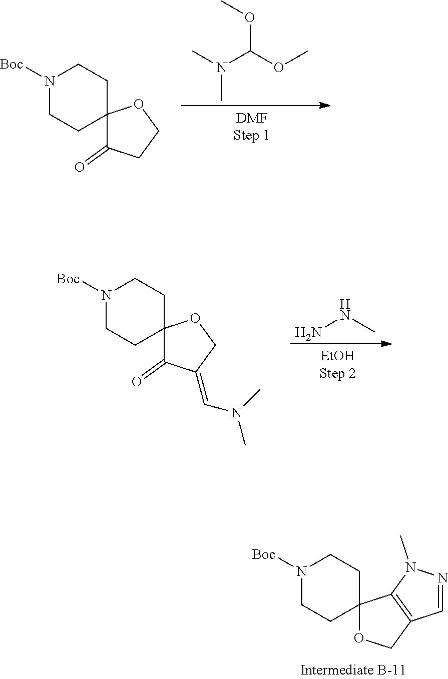

Step 1: tert-Butyl (E)-3-((dimethylamino)methylene)-4-oxo-1-oxa-8-azaspiro[4.5]decane-8-carboxylate. To a glass vial with a stir bar was added tert-butyl 4-oxo-1-oxa-8-azaspiro[4.5]decane-8-carboxylate (200 mg, 0.78 mmol) and (dimethoxymethyl)dimethylamine (312 μL, 2.35 mmol) in DMF (1.6 mL). The reaction mixture was stirred at 135° C. for 16 h. The reaction was quenched by H₂O (3 mL) and extracted with EtOAc (5 mL). The organic extract was washed with brine (3 mL), dried over Na₂SO₄, and concentrated to give tert-butyl (E)-3-((dimethylamino)methylene)-4-oxo-1-oxa-8-azaspiro[4.5]decane-8-carboxylate.

Step 2: tert-Butyl 1-methyl-1,4-dihydrospiro[furo[3,4-c]pyrazole-6,4′-piperidine]-1′-carboxylate, Intermediate B-11. To a glass vial with a stir bar was added tert-butyl (E)-3-((dimethylamino)methylene)-4-oxo-1-oxa-8-azaspiro[4.5]decane-8-carboxylate (243 mg, 0.78 mmol), methyl hydrazine (54 μL, 1.02 mmol) and EtOH (2.61 mL). The reaction mixture was heated to 80° C. and stirred 72 h. The reaction mixture was concentrated. The crude material was purified by silica gel chromatography, eluting with a gradient of 0-8% MeOH in DCM, to provide Intermediate B-11. MS (ESI) m/z: 294.2 (M+H)⁺.

### tert-Butyl 2-(oxetan-3-yl)-2,8-diazaspiro[4.5]decane-8-carboxylate (Intermediate B-12)

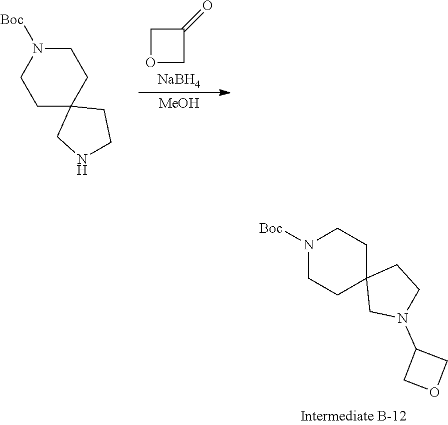

tert-Butyl 2-(oxetan-3-yl)-2,8-diazaspiro[4.5]decane-8-carboxylate, Intermediate B-12. To a glass vial with a stir bar was added 3-oxetanone (90 mg, 1.25 mmol), tert-butyl 2,8-diazaspiro[4.5]decane-8-carboxylate (200 mg, 0.83 mmol) and MeOH (4.2 mL). Sodium borohydride (63 mg, 1.66 mmol) was added at rt and stirred for 16 h. The reaction mixture was quenched by sat. aq. NH₄Cl solution (4 mL) and extracted with EtOAc (3 mL×3). The combined organic extracts were washed with brine, dried through Na₂SO₄, and concentrated to provide Intermediate B-12. MS (ESI) m/z: 297.2 (M+H)⁻.

### 2-Methylspiro[isoindoline-1,4′-piperidine] (Intermediate B-13)

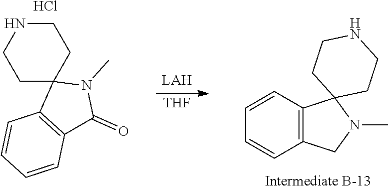 [SMILES: Cl ; CN1C(=O)c2ccccc2C12CCNCC2 ; CN1Cc2ccccc2C12CCNCC2 ; "THF"]

2-Methylspiro[isoindoline-1,4′-piperidine], Intermediate B-13. To a solution of 2-methylspiro[isoindoline-1,4′-piperidin]-3-one hydrochloride (300 mg, 1.19 mmol) in THF (5.9 mL) was added LAH (2 M in THF, 1.78 mL, 3.56 mmol) slowly at 0° C. After 44 h, the reaction was quenched by H₂O (0.15 mL) at 0° C., followed by addition of 15% NaOH aq. solution (0.15 mL) and H₂O (0.45 mL). The resulting mixture was stirred at rt for 15 min, and MgSO₄ was added while stirring. The mixture was diluted with EtOAc (10 mL), filtered, and concentrated to obtain Intermediate B-13. MS (ESI) m/z: 203.2 (M+H)⁺.

### 3-Methyl-1-oxa-8-azaspiro[4.5]decan-3-ol (Intermediate B-14)

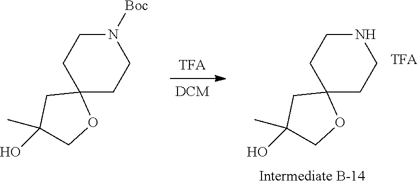 [SMILES: CC1(O)COC2(CCN(C(=O)OC(C)(C)C)CC2)C1 ; CC1(O)COC2(CCNCC2)C1]

3-Methyl-1-oxa-8-azaspiro[4.5]decan-3-ol (TFA salt), Intermediate B-14. To a solution of tert-butyl 3-hydroxy-3-methyl-1-oxa-8-azaspiro[4.5]decane-8-carboxylate (1.0 g, 3.69 mmol) in DCM (20 mL) was added TFA (8.0 mL, 104 mmol). The reaction mixture was stirred at rt for 3 h and the solvent was removed in vacuo. The residue was washed with ether and dried to afford Intermediate B-14, as a TFA salt. MS (ESI) m/z: 172.4 (M+H)⁺. ¹H NMR (400 MHz, DMSO-d₆) δ 1.25 (s, 3H) 1.66-1.81 (m, 4H) 1.87 (dd, J=13.05, 1.01 Hz, 1H) 2.03 (dt, J=14.07, 3.76 Hz, 1H) 2.99-3.15 (m, 4H) 3.53 (d, J=8.94 Hz, 1H) 3.64 (dd, J=8.82, 0.95 Hz, 1H) 8.53 (br s, 1H) 8.61 (br s, 1H).

The intermediates in the table below were prepared in a fashion similar to that described above.

TABLE 1-3

| | | | MS (ESI) |
|---|---|---|---|
| Int. No. | Chemical Structure | Name | m/z: (M + H)⁺ |
| B-15 | 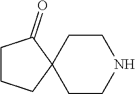 [SMILES: O=C1CCCC12CCNCC2] | 8-azaspiro[4.5]decan-1-one | 154.1 |
| B-16 | 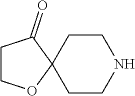 [SMILES: O=C1CCOC12CCNCC2] | 1-oxa-8-azaspiro[4.5]decan- 4-one | 156.4 |
| B-17 | 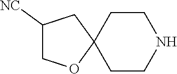 [SMILES: N#CC1COC2(CCNCC2)C1] | 1-oxa-8-azaspiro[4.5]decane- 3-carbonitrile | 167.4 |

### 4-Methyl-7H-spiro[furo[3,4-b]pyridine-5,4′-piperidine] (Intermediate B-19)

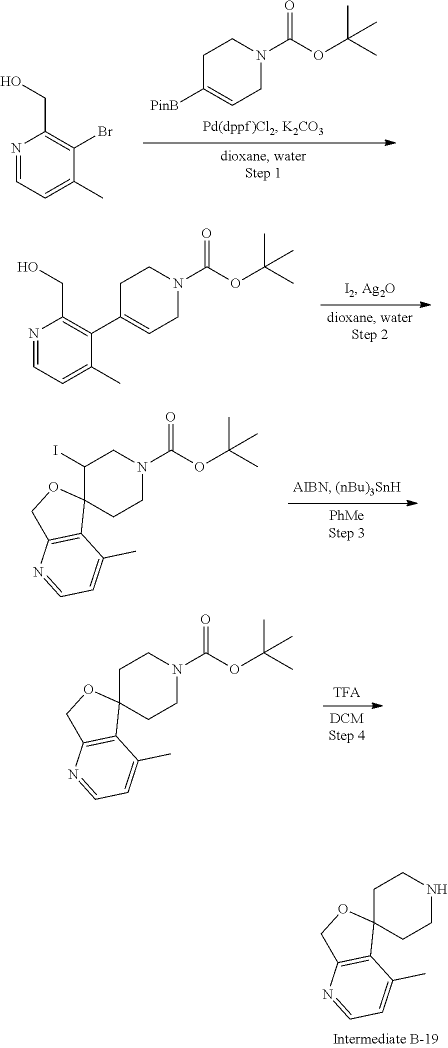

Step 1: tert-Butyl 2-(hydroxymethyl)-4-methyl-3′,6′-dihydro-[3,4′-bipyridine]-1′(2′H)-carboxylate. A mixture of (3-bromo-4-methylpyridin-2-yl)methanol (1.0 g, 4.95 mmol), tert-butyl 4-(4,4,5,5-tetramethyl-1,3,2-dioxaborolan-2-yl)-3,6-dihydropyridine-1 (2H) carboxylate (2.3 g, 7.42 mmol,), Pd(dppf)Cl₂ (404 mg, 0.50 mmol), K₂CO₃ (2.05 g, 14.85 mmol), 1,4-dioxane (25 mL), and H₂O (2.5 mL) was purged with N₂ for 10 min. The resulting mixture was heated at 100° C. for 3 h. The reaction was concentrated and purified by silica gel chromatography, eluting with a gradient of 0% to 50% (3:1) EtOAc/EtOH in heptane to give tert-butyl 2-(hydroxymethyl)-4-methyl-3′,6′-dihydro-[3,4′-bipyridine]-1′(2′H)-carboxylate. MS (ESI) m/z: 305.2 (M+H)⁺.

Step 2: tert-Butyl 3′-iodo-4-methyl-7H-spiro[furo[3,4-b]pyridine-5,4′-piperidine]-1′-carboxylate. To a stirring solution of tert-butyl 2-(hydroxymethyl)-4-methyl-3′,6′-dihydro-[3,4′-bipyridine]-1′(2′H)-carboxylate (1.0 g, 3.29 mmol) in dioxane (7 mL) and H₂O (1 mL) was added I₂ (1.25 g, 4.93 mmol). The mixture was stirred for 45 min, then Ag₂O (1.14 g, 4.93 mmol) was added. The mixture was stirred at rt for 48 h. Then, the reaction was filtered through celite, concentrated, and purified by silica gel chromatography, eluting with a gradient of 0% to 50% (3:1) EtOAc/EtOH in heptane to give tert-butyl 3′-iodo-4-methyl-7H-spiro[furo[3,4-b]pyridine-5,4′-piperidine]-1′-carboxylate. MS (ESI) m/z: 431.1 (M+H)⁺.

Step 3: tert-Butyl 4-methyl-7H-spiro[furo[3,4-b]pyridine-5,4′-piperidine]-1′-carboxylate. To a solution of tert-butyl 3′-iodo-4-methyl-7H-spiro[furo[3,4-b]pyridine-5,4′-piperidine]-1′-carboxylate (620 mg, 1.44 mmol) in toluene (14 mL) was added AIBN (473 mg, 2.9 mmol). The mixture was purged with N₂ for 10 min, then tri-n-butyltin hydride (1.15 mL, 4.32 mmol) was added and stirred at rt for 16 h. The reaction was concentrated and purified by silica gel chromatography, eluting with a gradient of 0% to 50% (3:1) EtOAc/EtOH in heptane to give tert-butyl 4-methyl-7H-spiro[furo[3,4-b]pyridine-5,4′-piperidine]-1′-carboxylate. MS (ESI) m/z: 305.2 (M+H)⁺.

Step 4: 4-Methyl-7H-spiro[furo[3,4-b]pyridine-5,4′-piperidine] (TFA salt), Intermediate B-19. To a solution of tert-butyl 4-methyl-7H-spiro[furo[3,4-b]pyridine-5,4′-piperidine]-1′-carboxylate (439 mg, 1.44 mmol) in DCM (5 mL) was added TFA (0.56 mL, 7.23 mmol) and the resulting mixture was stirred at rt for 15 min. The mixture was concentrated to afford 4-methyl-7H-spiro[furo[3,4-b]pyridine-5,4′-piperidine], which was used in the following stop as is assuming quantitative yield Intermediate B-19, as a TFA salt. MS (ESI) m/z: 241.1 (M+H)⁺.

The intermediate in the table below was prepared in a fashion similar to that described above.

TABLE 1-4

| | | | MS (ESI) |
|---|---|---|---|
| Int. No. | Chemical Structure | Name | m/z: (M + H)⁺ |
| B-27 | 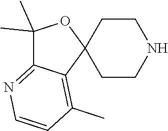 [SMILES: Cc1ccnc2c1C1(CCNCC1)OC2(C)C] | 4,7,7-trimethyl-7H-spiro [furo[3,4-b]pyridine-5.4′- piperidine] | 233.2 |

### ((2R,4R)-2,3′-Dimethyl-6′,7′-dihydrospiro[piperidine-4,4′-pyrazolo[5,1-c][1,4]oxazin]-7-yl) methanol (Intermediate B-20)

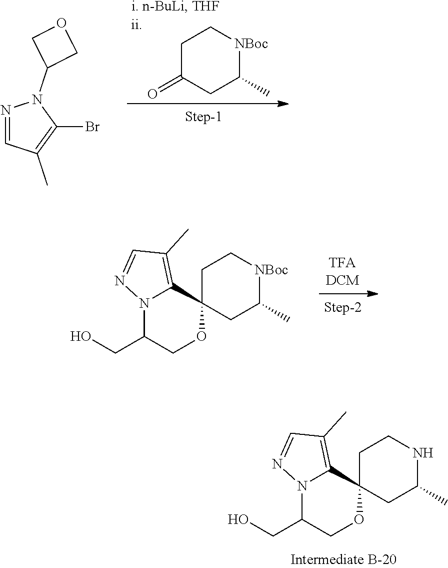

Step 1: tert-Butyl(2R,4R)-7′-(hydroxymethyl)-2,3′-dimethyl-6′,7′-dihydrospiro[piperidine-4,4′-pyrazolo[5,1-c][1,4]oxazine]-1-carboxylate. A solution of 5-bromo-4-methyl-1-(oxetan-3-yl)-1H-pyrazole (5.0 g, 23 mmol) in THF (200 mL) was cooled to −78° C. Then, n-BuLi (2.5 M in hexanes, 11.06 mL, 27.6 mmol) was added dropwise. The mixture was stirred at this temperature for 10 min, then a solution of tert-butyl (R)-2-methyl-4-oxopiperidine-1-carboxylate (5.40 g, 25.3 mmol) in THE (8 mL) was added. The mixture was allowed to warm up to rt over 2 h. The reaction was quenched with brine and extracted with EtOAc. The organic extract was concentrated and purified by silica gel chromatography, eluting with a gradient of 0% to 15% MeOH in DCM to give tert-butyl(2R,4R)-7′-(hydroxymethyl)-2,3′-dimethyl-6′,7′-dihydrospiro[piperidine-4,4′-pyrazolo[5,1-c][1,4]oxazine]-1-carboxylate. MS (ESI) m/z: 352.2 (M+H)⁺.

Step 2: ((2R,4R)-2,3′-Dimethyl-6′,7-dihydrospiro[piperidine-4,4′-pyrazolo[5,1-c][1,4]oxazin]-7′-yl)methanol (TFA salt), Intermediate B-20. To a solution of tert-butyl(2R,4R)-7′-(hydroxymethyl)-2,3′-dimethyl-6′,7′-dihydrospiro[piperidine-4,4′-pyrazolo[5,1-c][1,4]oxazine]-1-carboxylate (1.0 g, 2.8 mmol) in DCM (10 mL) was added TFA (10 mL, 130 mmol) and the resulting mixture was stirred at rt for 20 min. The reaction was concentrated to afford Intermediate B-20, as a TFA salt. MS (ESI) m/z: 252.2 (M+H)⁺.

### (2R,4R)-7′-(Fluoromethyl)-2,3′-dimethyl-6′,7′-dihydrospiro[piperidine-4,4′-pyrazolo[5.1-c][1,4]oxazine] (Intermediate B-21)

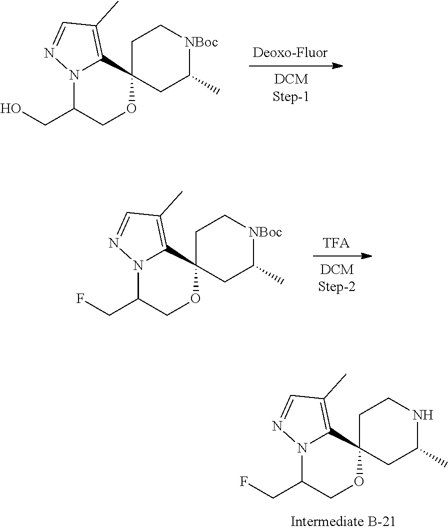

Step 1: tert-Butyl(2R,4R)-7-(fluoromethyl)-2,3′-dimethyl-6′,7′-dihydrospiro[piperidine-4,4′-pyrazolo[5,1-c][1,4]oxazine]-1-carboxylate. To a solution of Deoxo-Fluor (4.72 mL, 21.34 mmol) in DCM (12 mL) at −78° C. was added a solution of tert-butyl(2R,4R)-7′-(hydroxymethyl)-2,3′-dimethyl-6′,7′-dihydrospiro[piperidine-4,4′-pyrazolo[5,1-c][1,4]oxazine]-1-carboxylate (3.0 g, 8.54 mmol) in DCM (40 mL). The mixture was allowed to warm up to rt and stirred for 16 h. The reaction was Quenched with aq. satd. NaHCO₃ and extracted with EtOAc. The organics were purified by silica gel chromatography, eluting with 0-100% EtOAc in heptane to give tert-buty 1 (2R,4R)-7′-(fluoromethyl)-2,3′-dimethyl-6′,7-dihydrospiro[piperidine-4,4′-pyrazolo[5,1-c][1,4]oxazine]-1-carboxylate. MS (ESI) m/z: 354.1 (M+H)⁺.

Step 2: (2R,4R)-7′-(Fluoromethyl)-2,3′-dimethyl-6′,7-dihydrospiro[piperidine-4,4′-pyrazolo[5,1-c][1,4]oxazine] (TFA salt), Intermediate B-21. To a solution of tert-butyl(2R,4R)-7′-(fluoromethyl)-2,3′-dimethyl-6′,7′-dihydrospiro[piperidine-4,4′-pyrazolo[5,1-c][1,4]oxazine]-1-carboxylate (1.0 g, 2.83 mmol) in DCM (10 mL) was added TFA (10 mL, 130 mmol) and the resulting mixture was stirred at rt for 20 min. The reaction was concentrated to afford Intermediate B-21, as a TFA salt. MS (ESI) m/z: 254.2 (M+H)⁺.

### (2R,4R)-7′-(Methoxymethyl)-2,3′-dimethyl-6′,7′-dihydrospiro[piperidine-4,4′-pyrazolo[5,1-c][1,4]oxazine] (Intermediate B-22)

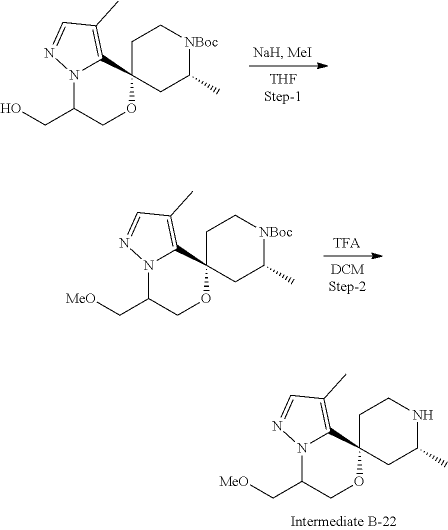

Step 1: tert-Butyl (2R,4R)-7-(methoxymethyl)-2,3′-dimethyl-6′,7′-dihydrospiro[piperidine-4,4′-pyrazolo[5,1-c][1,4]oxazine]-1-carboxylate. To a solution of tert-butyl(2R,4R)-7′-(hydroxymethyl)-2,3′-dimethyl-6′,7′-dihydrospiro[piperidine-4,4′-pyrazolo[5,1-c][1,4]oxazine]-1-carboxylate (3.0 g. 8.5 mmol) in THF (100 mL) at −78° C. was added sodium hydride (60% w/w, 410 mg, 17.1 mmol). The mixture was allowed to slowly warm up to 0° C. for 20 min, then iodomethane (6.06 g, 42.7 mmol) was added. The reaction was warmed to rt over 3 h. The reaction was quenched with H₂O (200 mL) and extracted with EtOAc (3×100 mL). The organics were purified by silica gel chromatography, eluting with a gradient of 0% to 100% EtOAc in heptane to give tert-butyl(2R,4R)-7′-(methoxymethyl)-2,3′-dimethyl-6′,7-dihydrospiro[piperidine-4,4′-pyrazolo[5,1-c]][1,4]oxazine]-1-carboxylate. MS (ESI) m/z: 366.1 (M+H)¹.

Step 2: (2R,4R)-7′-(Methoxymethyl)-2,3′-dimethyl-6′,7′-dihydrospiro[piperidine-4,4′-pyrazolo[5,1-c][1,4]oxazine], Intermediate B-22. To a solution of tert-butyl(2R,4R)-7′-(methoxymethyl)-2,3′-dimethyl-6′,7′-dihydrospiro[piperidine-4,4′-pyrazolo[5,1-c][1,4]oxazine]-1-carboxylate (1.0 g. 2.7 mmol) in DCM (10 mL) was added TFA (10 mL, 130 mmol) and the resulting mixture was stirred at rt for 20 min. The reaction was concentrated to afford Intermediate B-22 as a TFA salt. MS (ESI) m/z: 266.1 (M+H)⁺.

### (3′-Methyl-6′,7′-dihydrospiro[piperidine-4,4′-pyrazolo[5,1-c][1,4]oxazin]-7′-yl)methanol (Intermediate B-23)

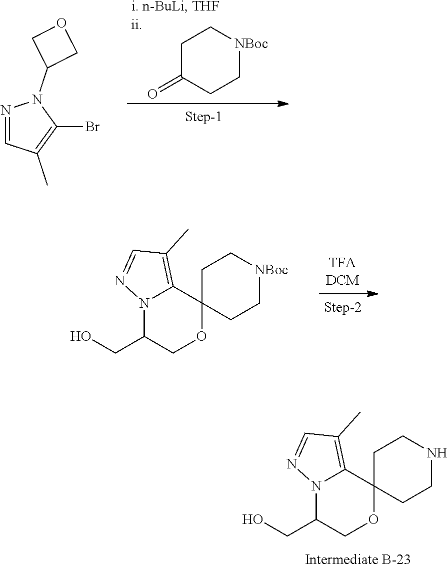

Step 1: tert-Butyl 7′-(hydroxymethyl)-3′-methyl-6′,7′-dihydrospiro[piperidine-4,4′-pyrazolo[5,1-c][1,4]oxazine]-1-carboxylate. A solution of 5-bromo-4-methyl-1-(oxetan-3-yl)-1H-pyrazole (2.0 g, 9.21 mmol) in THF (92 mL) was cooled to −78° C. and n-BuLi (2.5 M in hexanes, 4.42 mL, 11.1 mmol) was added dropwise. The mixture was stirred at −78° C. for 10 min, then a solution of tert-butyl 4-oxopiperidine-1-carboxylate (2.02 g, 10.1 mmol) in THF (8 mL) was added. The mixture was allowed to warm up to rt over 2 h. The reaction was quenched with brine and extracted with EtOAc. The combined organic extracts were concentrated and purified by silica gel chromatography, eluting with a gradient of 0% to 15% MeOH in DCM to give tert-butyl 7′-(hydroxymethyl)-3′-methyl-6′,7′-dihydrospiro[piperidine-4,4′-pyrazolo[5,1-c][1,4]oxazine]-1-carboxylate. MS (ESI) m/z: 338.1 (M+H)⁺.

Step 2: (3′-Methyl-6′,7′-dihydrospiro[piperidine-4,4′-pyrazolo[5,1-c][1,4]oxazin]-7-yl)methanol (TFA salt), Intermediate B-23. To a solution of tert-butyl 7′-(hydroxymethyl)-3′-methyl-6′,7′-dibydrospirofpiperidine-4,4′-pyrazolo[5,1-c][1,4]oxazine]-1-carboxylate (400 mg, 1.2 mmol) in DCM (5 mL) was added TFA (2.5 mL, 32.5 mmol) and the resulting mixture was stirred at rt for 20 min. The reaction was concentrated to afford Intermediate B-23, as a TFA salt. MS (ESI) m/z: 238.1 (M+H)¹.

### 7′-(Methoxymethyl)-3′-methyl-6′,7′-dihydrospiro[piperidine-4,4′-pyrazolo[5,1-c][1,4]oxazine] (Intermediate B-24)

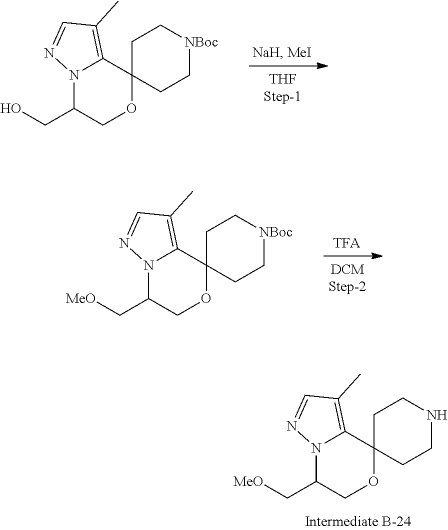 [SMILES: Cc1cnn2c1C1(CCN(C(=O)OC(C)(C)C)CC1)OCC2CO ; COCC1COC2(CCN(C(=O)OC(C)(C)C)CC2)c2c(C)cnn21 ; COCC1COC2(CCNCC2)c2c(C)cnn21]

Step 1: tert-Butyl 7′-(methoxymethyl)-3′-methyl-6′,7′-dihydrospiro[piperidine-4,4′-pyrazolo[5,1-c][1,4]oxazine]-1-carboxylate. To a solution of tert-butyl 7-(hydroxymethyl)-3′-methyl-6′,7′-dihydrospiro[piperidine-4,4′-pyrazolo[5,1-c][1,4]oxazine]-1-carboxylate (500 mg, 1.5 mmol) in THF (15 mL) at −78° C. was added sodium hydride (60% w/w, 71 mg, 3.0 mmol). The mixture was allowed to slowly warm up to 0° C. for 20 min, then iodomethane (1.05 g, 7.41 mmol) was added. The reaction was warmed to rt over 3 h. The reaction was quenched with H₂O and extracted with EtOAc. The organics were purified by silica gel chromatography, eluting with a gradient of 0% to 100% EtOAc in heptane to give tert-butyl 7′-(methoxymethyl)-3′-methyl-6′,7′-dihydrospiro[piperidine-4,4′-pyrazolo[5,1-c][1,4]oxazine]-1-carboxylate. MS (ESI) m/z: 352.2 (M+H)⁺.

Step 2: 7′-(Methoxymethyl)-3′-methyl-6′,7′-dihydrospiro[piperidine-4,4′-pyrazolo[5,1-c][1,4]oxazine] (TFA salt), Intermediate B-24. To a solution of tert-butyl 7′-(methoxymethyl)-3′-methyl-6′,7′-dihydrospiro[piperidine-4,4′-pyrazolo[5,1-c][1,4]oxazine]-1-carboxylate (440 mg, 1.3 mmol) in DCM (10 mL) was added TFA (2.5 mL, 33 mmol) and the resulting mixture was stirred at rt for 20 min. The reaction was concentrated to afford Intermediate B-24 as a TFA salt. MS (ESI) m/z: 252.1 (M+H)⁺.

### 7′-(Fluoromethyl)-3′-methyl-6′,7′-dihydrospiro[piperidine-4,4′-pyrazolo[5.1-c][1,4]oxazine] (Intermediate B-25)

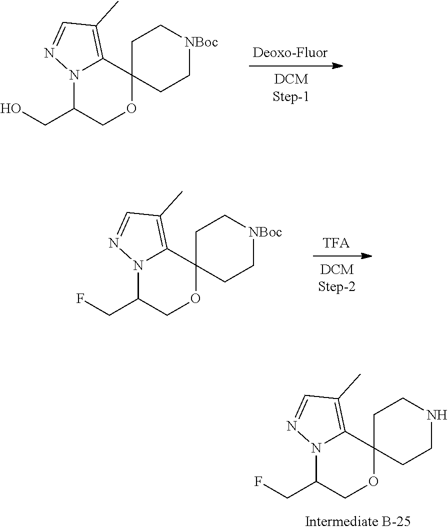 [SMILES: Cc1cnn2c1C1(CCN(C(=O)OC(C)(C)C)CC1)OCC2CO ; Cc1cnn2c1C1(CCN(C(=O)OC(C)(C)C)CC1)OCC2CF ; Cc1cnn2c1C1(CCNCC1)OCC2CF]

Step 1: tert-Butyl 7′-(fluoromethyl)-3′-methyl-6′,7′-dihydrospiro[piperidine-4,4′-pyrazolo[5,1-c][1,4]oxazine]-1-carboxylate. To a solution of Deoxo-Fluor (0.820 mL, 3.70 mmol) in DCM (12 mL) at −78° C. was added a solution of tert-butyl 7′-(hydroxymethyl)-3′-methyl-6′,7′-dihydrospiro[piperidine-4,4′-pyrazolo[5,1-c][1,4]oxazine]-1-carboxylate (500 mg, 1.5 mmol) in DCM (8 mL). The mixture was allowed to slowly warm up and stirred for 16 h. The reaction was quenched with sat. NaHCO₃ and extracted with EtOAc. The organics were purified by silica gel chromatography, eluting with a gradient of 0% to 100% EtOAc in heptane to give tert-butyl 7-(fluoromethyl)-3′-methyl-6′,7′-dihydrospiro[piperidine-4,4′-pyrazolo[5,1-c][1.4]oxazine]-1-carboxylate. MS (ESI) m/z: 340.2 (M+H)⁺.

Step 2: 7′-(Fluoromethyl)-3′-methyl-6′,7′-dihydrospiro[piperidine-4,4′-pyrazolo[5,1-c][1,4]oxazine] (TFA salt), Intermediate B-25. To a solution of tert-butyl 7′-(fluoromethyl)-3′-methyl-6′,7′-dihydrospiro[piperidine-4,4′-pyrazolo[5,1-c][1,4]oxazine]-1-carboxylate (400 mg, 1.18 mmol) in DCM (10 mL) was added TFA (2.5 mL, 32.5 mmol) and the resulting mixture was stirred at rt for 20 min. The reaction was concentrated to afford Intermediate B-25, as a TFA salt. MS (ESI) m/z: 240.2 (M+H)⁺.

The intermediate in the table below was prepared in a fashion similar to that described above.

TABLE 1-5

| | | | MS (ESI) |
|---|---|---|---|
| Int. No. | Chemical Structure | Name | m/z: (M + H)⁺ |
| B-28 | 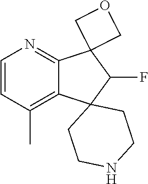 [SMILES: Cc1ccnc2c1C1(CCNCC1)C(F)C21COC1] | 6′-fluoro-4′-methyl-6'H- dispirofoxetane-3,7′- cyclopentalbjpyridine- 5′,4″-piperidine] | 263.2 |

### (4-Methyl-2H-spiro[furo[2,3-b]pyridine-3,4′-piperidine] (Intermediate B-26)

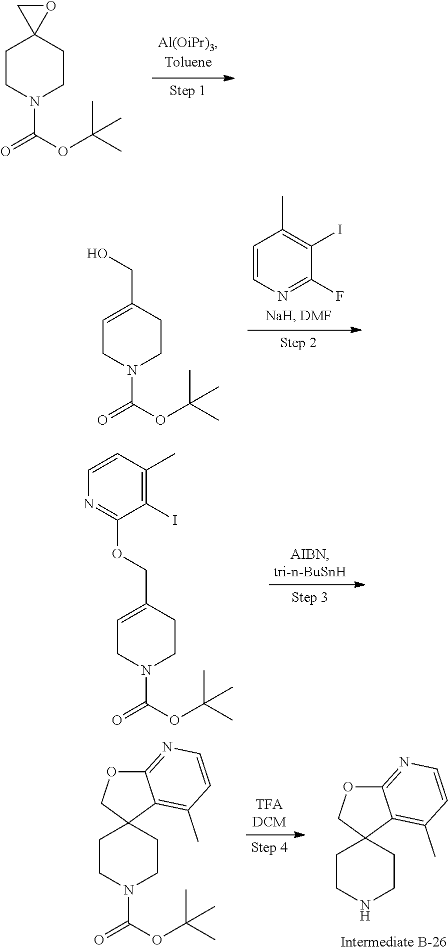

Step 1: tert-Butyl 4-(hydroxymethyl)-3,6-dihydropyridine-1 (2H)-carboxylate. To a solution of tert-butyl 1-oxa-6-azaspiro[2.5]octane-6-carboxylate (20 g, 94 mmol) in toluene (400 mL) was added aluminum isopropoxide (28.7 g, 141 mmol) at 25° C. and the resulting reaction mixture was stirred at 110° C. for 24 h. The reaction mixture was diluted with EtOAc (250 mL) and washed with 1.5 N HCl solution (2×100 mL), followed by brine. The organic layer was dried over Na₂SO₄, filtered, and concentrated. The crude material was purified by silica gel chromatography eluting with a gradient of 35% to 40% EtOAc in hexanes to obtain tert-butyl 4-(hydroxymethyl)-3,6-dihydropyridine-1(2H)-carboxylate. ¹H NMR (DMSO-d₆, 400 MHz) δ 5.57 (s, 1H), 4.76 (t, J=5.5 Hz, 1H), 3.82 (q, J=5.3, 3.8 Hz, 4H), 3.40 (t, J=5.8 Hz, 2H), 2.01-1.94 (m, 2H), 1.41 (s, 9H). MS (ESI) m/z: 114.2 (M−Boc)⁺.

Step 2: tert-Butyl 4-(((3-iodo-4-methylpyridin-2-yl)oxy)methyl)-3,6-dihydropyridine-1(2H)-carboxylate. To solution of tert-butyl 4-(hydroxymethyl)-3,6-dihydropyridine-1(2H)-carboxylate (2 g. 9.4 mmol) in DMF (30 mL) was added NaH (60% dispersion in mineral oil, 474 mg, 11.3 mmol) portion wise at 0° C. The resulting reaction mixture was allowed to stir at rt for 1 h. Then, 2-fluoro-3-iodo-4-methylpyridine (2 g, 8.4 mmol) was added as a solid at 0° C. and the resulting reaction mixture was stirred at rt for 16 h. The reaction mixture was quenched with a 10% NaHCO₃ solution (40 mL) and extracted with EtOAc (2×100 mL). The combined organic extracts were washed with brine, dried over Na₂SO₄, filtered, and concentrated under vacuum. The crude product was purified by silica gel chromatography eluting with a gradient of 5% to 10% EtOAc in hexanes to give tert-butyl 4-(((3-iodo-4-methylpyridin-2-yl)oxy)methyl)-3,6-dihydropyridine-1 (2H)-carboxylate. ¹H NMR (DMSO-d₆, 400 MHz) δ 8.07 (d, J=2.1 Hz, 1H), 7.96 (dd, J=2.1, 1.0 Hz, 1H), 5.78 (d, J=11.6 Hz, 1H), 4.71 (d, J=2.1 Hz, 2H), 3.86 (s, 2H), 3.45 (t, J=5.8 Hz, 2H), 2.19 (s, 3H), 2.13 (d, J=6.6 Hz, 2H), 1.41 (s, 9H). MS (ESI) m/z: 431.1 (M+H)⁺.

Step 3: tert-Butyl 4-methyl-2H-spiro[furo[2,3-b]pyridine-3,4′-piperidine]-1′-carboxylate. To a degassed solution of tert-butyl 4-(((3-iodo-4-methylpyridin-2-yl)oxy)methyl)-3,6-dihydropyridine-1(2H)-carboxylate (2.23 g, 5.18 mmol) in toluene (30 mL) was added AIBN (170 mg, 1.04 mmol) followed by tri-n-butyltin hydride (4.84 mL, 18.14 mmol) at rt. The resulting reaction mixture was heated at 110° C. for 24 h. The reaction mixture was diluted with EtOAc (100 mL) and washed with 10% KF solution (2×80 mL). The solid precipitate was filtered through celite. The organic extracts were washed with brine, dried over Na₂SO₄, filtered, and concentrated under vacuum. The crude product was purified by silica gel chromatography eluting with a gradient of 50% to 60% EtOAc in hexanes to obtain tert-butyl 4-methyl-2H-spiro[furo[2,3-b]pyridine-3,4′-piperidine]-1′-carboxylate. ¹H NMR (DMSO-d₆, 400 MHz) δ 7.76 (dd, J=2.2, 1.0 Hz, 1H), 7.56 (d, J=2.2 Hz, 1H), 4.41 (s, 2H), 3.85 (d, J=13.6 Hz, 2H), 2.97 (s, 2H), 2.19 (s, 3H), 1.77-1.60 (m, 4H), 1.43 (s, 9H). MS (ESI) m/z: 305.2 (M+H)⁺.

Step 4: 4-Methyl-2H-spiro[furo[2,3-b]pyridine-3, 4-piperidine] (TFA salt), Intermediate B-26. To a solution of tert-butyl 4-methyl-2H-spiro[furo[2,3-b]pyridine-3,4′-piperidine]-1′-carboxylate (230 mg, 0.76 mmol) in DCM (4.60 mL) was added TFA (0.70 mL, 9.07 mmol) dropwise at 0° C. The resulting pale-yellow solution was allowed to stir at rt for 2 h. The reaction mixture was evaporated to dryness and to that residue toluene (50 mL) was added and evaporated. The same process was repeated two more times to obtain Intermediate B-26, as a TFA salt which was taken forward without further purification. MS (ESI) m/z: 205.2 (M+H)⁺.

### Intermediate B29-4′-Methyldispiro[oxetane-3,7′-furo[3,4-b]pyridine-5′,4″-piperidine]

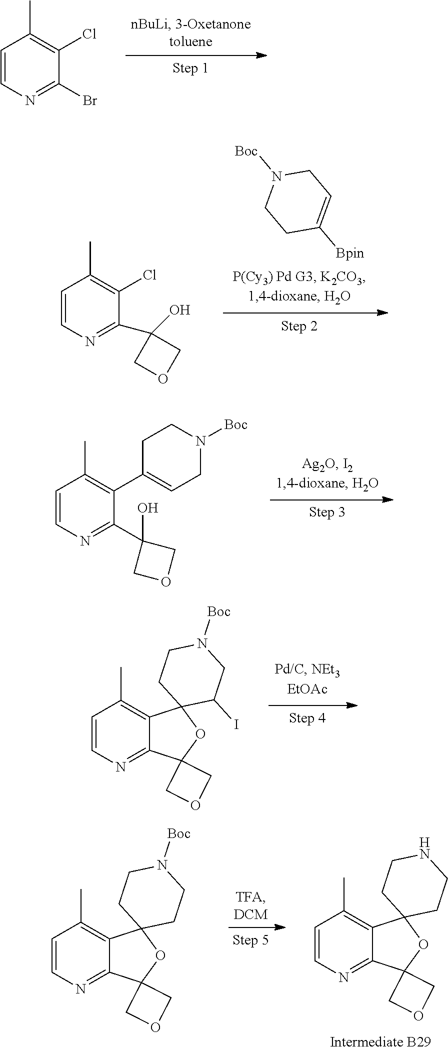

Step 1:3-(3-Chloro-4-methylpyridin-2-yl) oxetan-3-ol. To a mixture of 2-bromo-3-chloro-4-picoline (9.45 g, 45.8 mmol) and toluene (120 mL) at −78° C., was slowly added n-BuLi (2.5 M solution in hexanes, 19 mL, 47.5 mmol). The reaction mixture was stirred at −78° C. for 0.5 h. Then, 3-oxetanone (3.1 mL, 48.4 mmol) was added at −78° C., and the reaction mixture was stirred for 1 h. The reaction was warmed to rt and quenched by adding sat. aq. NH₄Cl solution (150 mL). The crude material was extracted with EtOAc (150 mL×3), washed with brine, dried through Na₂SO₄, and concentrated. The crude material was purified by silica gel chromatography eluting with a gradient of 10% to 50% acetone in heptane to provide 3-(3-chloro-4-methylpyridin-2-yl) oxetan-3-ol (6.8 g, 34.1 mmol, 74% yield). m/z (ESI): 200.1 (M+H)⁺. ¹H NMR (DMSO-d₆, 400 MHz) δ 8.37 (d, J=4.8 Hz, 1H), 7.40 (d, J=4.8 Hz, 1H), 6.45 (s, 1H), 5.15 (dd, J=6.9, 0.9 Hz, 2H), 4.68 (dd, J=6.9, 1.0 Hz, 2H). 2.38 (s, 3H).

Step 2: tert-Butyl 2-(3-hydroxyoxetan-3-yl)-4-methyl-3′,6′-dihydro-[3,4″bipyridine]-1′(2′H)-carboxylate. To a solution of 3,6-dihydro-2H-pyridine-1-N-boc-4-boronic acid pinacol ester (1.7 g. 5.5 mmol) and 3-(3-chloro-4-methylpyridin-2-yl) oxetan-3-ol (1.0 g, 5.0 mmol) in 1,4-dioxane (12 mL) and H₂O (1.2 mL) was added K₂CO₃ (2.1 g, 15.0 mmol, Sigma-Aldrich, Inc.) and P(Cy₃) Pd G3 dichloromethane adduct (326 mg, 0.50 mmol). The reaction mixture was bubbled with N₂ for 15 min, then stirred at 100° C. for 20 h. The reaction was quenched with sat. aq. NH₄Cl solution (10 mL) and extracted with EtOAc (10 mL×3). The combined organic extracts were washed with brine, dried over Na₂SO₄, and concentrated. The crude material was purified by chromatography eluting with a gradient of 10% to 60% acetone in heptane to provide ter-butyl 2-(3-hydroxyoxetan-3-yl)-4-methyl-3′,6′-dihydro-[3,4′-bipyridine]-1′(2′H)-carboxylate (1.25 g. 3.61 mmol, 72% yield). m/z (ESI): 347.0 (M+H)⁺.

Step 3: tert-Butyl 3″-iodo-4′-methyldispiro[oxetane-3,7′-furo[3,4-b]pyridine-5′,4″-piperidine]-1″-carboxylate. To a solution of tert-butyl 2-(3-hydroxyoxetan-3-yl)-4-methyl-3′,6′-dihydro-[3,4′-bipyridine]-1′(2′H)-carboxylate (1.8 g, 5.20 mmol) in 1,4-dioxane (80 mL) and H₂O (16 mL), was added iodine (4.72 g, 18.60 mmol), and the reaction mixture was stirred at rt for 30 min before silver oxide (4.82 g, 20.78 mmol.) was added and the reaction was stirred for 72 h. Then, the reaction was filtered, concentrated, and purified by chromatography with a gradient of 0% to 60% acetone in heptane to provide tert-butyl 3″-iodo-4′-methyldispiro[oxetane-3,7′-furo[3,4-b]pyridine-5′,4″-piperidine]-1″-carboxylate (1.84 g. 3.9 mmol, 75% yield). MS (ESI) m/z: 472.8 (M+H)⁺.

Step 4: tert-Butyl 4′-methyldispiro[oxetane-3,7′-furo[3,4-b]pyridine-5′,4″-piperidine]-1″-carboxylate. To a solution of the 1^{st} eluting isomer from Step 3 (1.84 g, 3.9 mmol) and TEA (2.2 mL, 15.7 mmol) in EtOAc (39 mL) was added 10 wt % Pd/C (1.4 g, 1.3 mmol), and the reaction was purged with N₂. Then, the reaction was charged with H₂ and stirred at rt for 48 h. The reaction mixture was filtered, concentrated, and purified by chromatography eluting with a gradient of 3-40% acetone in heptane to provide tert-butyl 4′-methyldispiro[oxetane-3,7′-furo[3,4-b]pyridine-5′,4″-piperidine]-1″-carboxylate (1.0 g, 2.89 mmol, 74% yield). m/z (ESI): 347.0 (M+H)⁺.

Step 5: 4′-Methyldispiro[oxetane-3,7′-furo[3,4-b]pyridine-5′,4″-piperidine], Intermediate B29. To a solution of tert-butyl 4′-methyldispiro[oxetane-3,7′-furo[3,4-b]pyridine-5′,4″-piperidine]-1″-carboxylate (280 mg, 0.81 mmol) in DCM (6 mL), was added TFA (0.52 mL, 6.7 mmol) at rt, and stirred for 3 h, then concentrated to provide Intermediate B29, as a TFA salt. m/z (ESD): 247.2 (M+H)⁺.

The intermediate in the table below were prepared in a fashion similar to that described above.

TABLE 1-6

| | | | MS (ESI) |
|---|---|---|---|
| Int. No. | Chemical Structure | Name | m/z: (M + H)⁺ |
| B-32 | 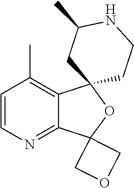 [SMILES: Cc1ccnc2c1[C@]1(CCN[C@H](C)C1)OC21COC1] | (2″R,5'R)-2″,4′-dimethyl- dispiro[oxetane-3,7′-furo[3,4- b]pyridine-5′,4″-piperidine] | 261.1 |

### Intermediate B30-4′-Methyl-6′H-dispiro[oxetane-3,7′-cyclopenta[b]pyridine-5′,4″-piperidin]-6′-one

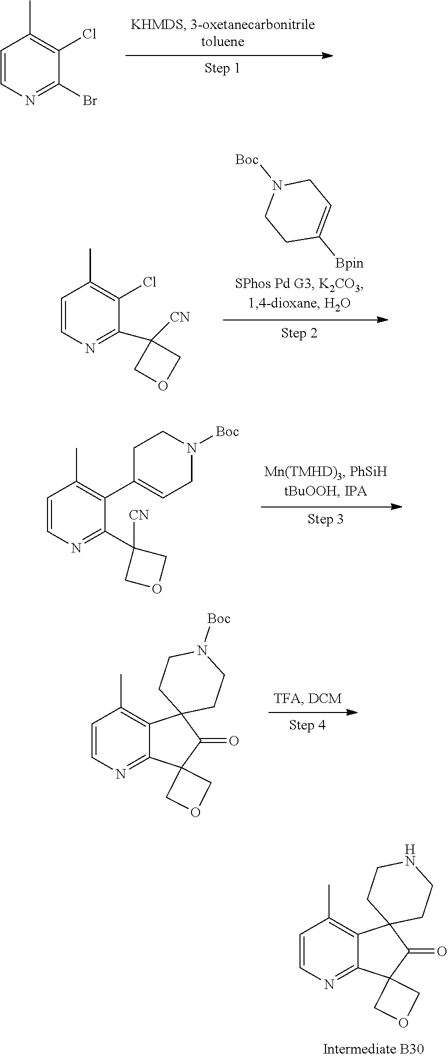

Step 1:3-(3-Chloro-4-methylpyridin-2-yl) oxetane-3-carbonitrile. To a solution of 2-bromo-3-chloro-4-picoline (2.5 g, 12.11 mmol) and 3-oxetanecarbonitrile (1.21 g. 14.53 mmol) in toluene at −78° C. was added KHMDS (0.5 M in toluene, 33.9 mL, 16.95 mmol) slowly. The reaction mixture was stirred at −78° C. for 2 h and quenched by adding sat. aq. NH₄Cl solution (150 mL). The crude material was extracted with EtOAc (150 mL×3), washed with brine, dried through Na₂SO₄, and concentrated. The crude material was purified by silica gel chromatography eluting with a gradient of 2% to 34% acetone in heptane to provide 3-(3-chloro-4-methylpyridin-2-yl) oxetane-3-carbonitrile (1.0 g, 4.8 mmol, 40% yield). m/z (ESI): 209.0 (M+H)⁺.

Step 2: tert-Butyl 2-(3-cyanooxetan-3-yl)-4-methyl-3′,6′-dihydro-[3,4′-bipyridine]-1′(2′H)-carboxylate. To a solution of 3,6-dihydro-2H-pyridine-1-N-boc-4-boronic acid pinacol ester (1.78 g, 5.7 mmol) and 3-(3-chloro-4-methylpyridin-2-yl) oxetane-3-carbonitrile (0.8 g, 3.83 mmol) in 1,4-dioxane (10 mL) and H₂O (1.0 mL) was added K₂CO₃ (1.59 g, 11.5 mmol) and Sphos Pd G3 (305 mg, 0.38 mmol). The reaction mixture was bubbled with N₂ for 15 min, then stirred at 90° C. for 18 h. The reaction was quenched with sat. aq. NH₄Cl solution (10 mL) and extracted with EtOAc (10 mL×3). The combined organic extracts were washed with brine, dried over Na₂SO₄, and concentrated. The crude material was purified by chromatography eluting with a gradient of 0% to 100% EtOAc in heptane to provide tert-butyl 2-(3-cyanooxetan-3-yl)-4-methyl-3′,6′-dihydro-[3,4′-bipyridine]-1′(2′H)-carboxylate (1.0 g. 2.81 mmol, 73% yield). m/z (ESI): 356.0 (M+H)⁺.

Step 3: tert-Butyl 4′-methyl-6′-oxo-6′H-dispiro[oxetane-3,7′-cyclopenta[b]pyridine-5′,4″-piperidine]-1″-carboxylate. To a mixture of tert-butyl 2-(3-cyanooxetan-3-yl)-4-methyl-3′,6′-dihydro-[3,4′-bipyridine]-1′(2′H)-carboxylate (430 mg, 1.21 mmol) and tris(2,2,6,6-tetramethyl-3,5-heptanedionato)manganese(iii) (146 mg, 0.242 mmol) in IPA (5 mL) at rt was added phenylsilane (0.89 mL, 7.26 mmol) and 1,1-dimethylethyl hydroperoxide (0.726 mL, 3.63 mmol) dropwise. The reaction mixture was stirred at rt for 3 h. The reaction was quenched with NH₄OH and extracted with EtOAc (10 mL×3). The combined organic extracts were washed with brine, dried over Na₂SO₄, and concentrated. The crude material was purified by prep HPLC to provide tert-butyl 4′-methyl-6′-oxo-6′H-dispiro[oxetane-3,7′-cyclopenta[b]pyridine-5′,4″-piperidine]-1″-carboxylate (180 mg, 0.50 mmol, 41% yield). m/z (ESI): 359.0 (M+H)⁺.

Step 4: 4′-Methyl-6′H-dispiro[oxetane-3,7-cyclopenta[b]pyridine-5′,4″-piperidin]-6′-one, Intermediate B30. To a solution of tert-butyl 4′-methyl-6′-oxo-6′H-dispiro[oxetane-3,7′-cyclopenta[b]pyridine-5′,4″-piperidine]-1″-carboxylate (180 mg, 0.50 mmol) in DCM (6 mL), was added TFA (0.5 mL) at rt, and stirred for 3 h, then concentrated to provide Intermediate B30, as a TFA salt. m/z (ESI): 259.1 (M+H)⁺.

### Intermediate B31-4′-Methyl-6′H-dispiro[oxetane-3,7′-cyclopenta[b]pyridine-5′,4″-piperidin]-6′-one

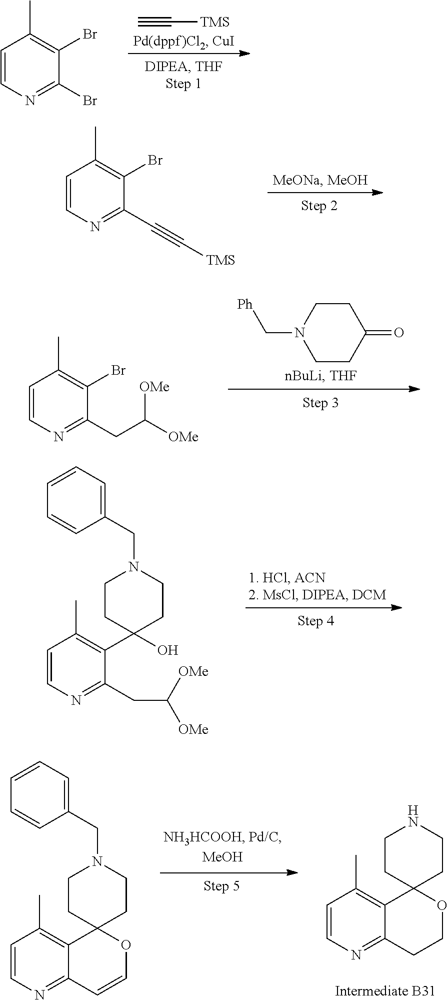

Step 1:3-Bromo-4-methyl-2-((trimethylsilyl)ethynyl)pyridine. To a mixture of 2,3-dibromo-4-methylpyridine (500 mg, 1.99 mmol), PdCl₂(PPb₃)₂ (140 mg, 0.19 mmol) and copper (I) iodide (40 mg, 0.21 mmol), in THF (20 mL), was added DIPEA (1.0 mL, 5.73 mmol). The reaction mixture was sparged with argon for 5 min before addition of trimethylsilylacetylene (0.31 mL, 2.21 mmol). The reaction mixture was stirred at rt for 16 h. The crude material was purified by silica gel chromatography eluting with a gradient of 5% to 100% EtOAc in heptane to provide 3-bromo-4-methyl-2-((trimethylsilyl)ethynyl)pyridine (380 mg, 1.42 mmol, 71% yield). m/z (ESI): 268.0/270.0 (M+H)⁺.

Step 2: 3-Bromo-2-(2,2-dimethoxyethyl)-4-methylpyridine. To a solution of 3-bromo-4-methyl-2-((trimethylsilyl) ethynyl)pyridine (380 mg, 1.42 mmol) in MeOH (3 mL) at rt was added sodium methoxide (25% in MeOH, 1 mL, 4.37 mmol). The reaction mixture was stirred at 60° C. for 48 h. The reaction mixture was poured into satd. NaHCO₃ solution and extracted with EtOAc (2×). The combined organic extracts were washed with brine, dried over Na₂SO₄, and concentrated. The crude material was purified by chromatography eluting with a gradient of 5% to 100% EtOAc in heptane to provide 3-bromo-2-(2,2-dimethoxyethyl)-4-methylpyridine (166 mg, 0.64 mmol, 45% yield). m/z (ESI): 260.1/262.0 (M+H)⁺.

Step 3: 1-Benzyl-4-(2-(2,2-dimethoxyethyl)-4-methylpyridin-3-yl)piperidin-4-ol. To a solution of 3-bromo-2-(2,2-dimethoxyethyl)-4-methylpyridine (166 mg, 0.64 mmol) in THF (6 mL) at −78° C. was slowly added n-BuLi (1.5 M in hexanes, 0.64 mL, 0.96 mmol). The reaction mixture was stirred at −78° C. for 5 min and then 1-benzylpiperidin-4-one (0.18 mL, 0.971 mmol) was added. Stirring at −78° C. was continued for 20 min and then at rt for 1 h. The reaction mixture was poured into satd. NaHCO₃ solution and extracted with EtOAc (2×). The combined organic extracts were washed with brine, dried over Na2SO4, and concentrated. The crude material was purified by chromatography eluting with a gradient of 5% to 100% EtOAc in heptane followed by 0% to 20% MeOH in EtOAc to provide 1-benzyl-4-(2-(2,2-dimethoxyethyl)-4-methylpyridin-3-yl)piperidin-4-ol (60 mg, 0.16 mmol, 25% yield). m/z (ESI): 371.3 (M+H)+.

Step 4: 1-Benzyl-4′-methylspiro[piperidine-4,5′-pyrano[4,3-b]pyridine]. To a solution of 1-benzyl-4-(2-(2,2-dimethoxyethyl)-4-methylpyridin-3-yl)piperidin-4-ol (60 mg, 0.16 mmol) in CH₃CN (2 mL) was added HCl (6 M, 0.6 mL) and the reaction mixture was heated at 60° C. for 18 h. The reaction mixture was poured into satd. NaHCO₃ solution and extracted with EtOAc (2×). The combined organic extracts were washed with brine, dried over Na₂SO₄, and concentrated. The residue was redissolved in DCM (2 mL) and DIPEA (0.05 mL, 0.31 mmol) followed by MsCl (10 mL, 0.12 mmol) were added. The reaction mixture was stirred at rt for 1 h and poured into satd NaHCO₃ solution and extracted with EtOAc (2×). The combined organic extracts were washed with brine, dried over Na₂SO₄, and concentrated. The crude material was purified by chromatography eluting with a gradient of 2% to 100% EtOAc in heptane to provide 1-benzyl-4′-methylspiro[piperidine-4,5′-pyrano[4,3-b]pyridine] (7 mg, 0.02 mmol, 37% yield). m/z (ESI): 307.2 (M+H)+.

Step 5: 4′-Methyl-6′H-dispiro[oxetane-3,7-cyclopenta[b]pyridine-5′,4″-piperidin]-6′-one, Intermediate 31. To a solution of 1-benzyl-4′-methylspiro[piperidine-4,5′-pyrano[4,3-b]pyridine] (27 mg, 0.09 mmol) in MeOH (2 mL) was added Pd/C (10 wt %, 19 mg, 0.018 mmol), and ammonium formate (38 mg, 6.84 mmol). The reaction was stirred at 60° C. for 18 h. The mixture was cooled to rt, filtered over celite, and concentrated to provide Intermediate B31. m/z (ESI): 219.2 (M+H)+.

### Intermediate B33-1-(S)-3′,7-Dimethyl-6′,7′-dihydrospiro[piperidine-4,4′-pyrazolo[5,1-c][1,4]oxazine]

### Intermediate B33-2-(R)-3′,7′-Dimethyl-6′,7-dihydrospiro[piperidine-4,4′-pyrazolo[5,1-c][1,4]oxazine]

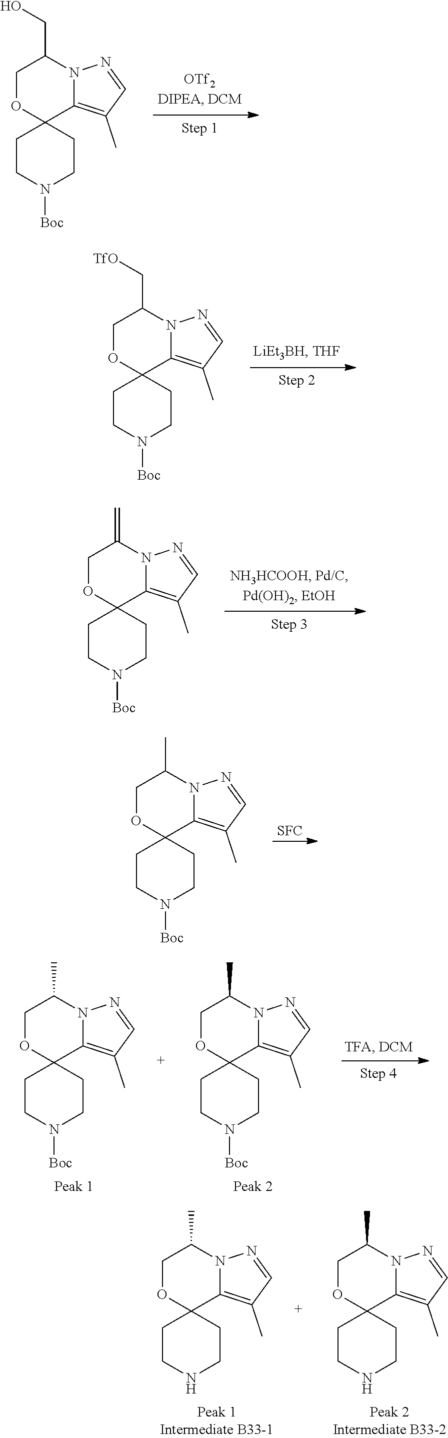

Step 1: tert-Butyl 3′-methyl-7′-((((trifluoromethyl)sulfonyl)oxy)methyl)-6′,7′-dihydrospiro[piperidine-4,4′-pyrazolo[5,1-c][1,4]oxazine]-1-carboxylate. To a solution of tert-butyl 7′-(hydroxymethyl)-3′-methyl-6′,7′-dihydrospiro[piperidine-4,4′-pyrazolo[5,1-c][1,4]oxazine]-1-carboxylate (Intermediate B23, step 1) (200 mg, 0.59 mmol) in DCM (10 mL) at −78° C. was added DIPEA (0.31 mL, 1.78 mmol), followed by OTf₂ (1 M in DCM, 0.71 mL, 0.71 mmol). The mixture was warmed to rt and stirred for 15 min. The mixture was quenched with satd. aq. NH₄Cl, extracted with DCM, dried with NaSO₄, and concentrated to give tert-butyl 3′-methyl-7′-((((trifluoromethyl)sulfonyl)oxy)methyl)-6′,7′-dihydrospiro[piperidine-4,4′-pyrazolo[5,1-c][1,4]oxazine]-1-carboxylate. m/z (ESI): 470.1 (M+H)⁺.

Step 2: tert-Butyl 3′-methyl-7-methylene-6′,7′-dihydrospiro[piperidine-4,4′-pyrazolo[5,1-c][1,4]oxazine]-1-carboxylate. To a solution of tert-butyl 3′-methyl-7′-((((trifluoromethyl)sulfonyl)oxy)methyl)-6′,7′-dihydrospiro[piperidine-4,4′-pyrazolo[5,1-c][1,4]oxazine]-1-carboxylate (2.78 g, 5.9 mmol) in THF (60 mL) at 0° C. was added lithium triethylborohydride, (1.0 M in THF, 12.0 mL, 12 mmol), and the reaction was stirred for 15 h at 0° C. The reaction was quenched with satd. aq. NaHCO₃, extracted with EtOAc, and purified by silica gel chromatography eluting with a gradient of 0% to 10% MeOH in DCM to give tert-butyl 3′-methyl-7′-methylene-6′,7′-dihydrospiro[piperidine-4,4′-pyrazolo[5,1-c][1,4]oxazine]-1-carboxylate (1.89 g, 5.92 mmol, 100% yield). m/z (ESI): 320.2 (M+H)⁺.

Step 3: tert-Butyl 3′,7′-dimethyl-6′,7′-dihydrospiro[piperidine-4,4′-pyrazolo[5,1-c][1,4]oxazine]-1-carboxylate. To a solution of tert-butyl 3′-methyl-7′-methylene-6′,7′-dihydrospiro[piperidine-4,4′-pyrazolo[5,1-c][1,4]oxazine]-1-carboxylate (1.89 g, 5.92 mmol), and EtOH (60 mL), was added Pd(OH)₂ on carbon (20 wt %, 1.25 g, 1.78 mmol), Pd/C (10 wt %, 1.89 g, 1.78 mmol), and ammonium formate (7.46 g, 118 mmol). The reaction was placed in a preheated aluminum block and kept at 50° C. for 2 h. The mixture was cooled to rt, filtered over celite, and concentrated. The residue was purified by silica gel chromatography, eluting with a gradient of 0% to 100% EtOAc in heptane to give tert-butyl 3′,7′-dimethyl-6′,7-dihydrospiro[piperidine-4,4′-pyrazolo[5,1-c][1,4]oxazine]-1-carboxylate (1.29 g, 4.01 mmol, 68% yield). m/z (ESI): 322.2 (M+H)⁺.

tert-Butyl 3′,7′-dimethyl-6′,7′-dihydrospiro[piperidine-4,4′-pyrazolo[5,1-c][1,4]oxazine]-1-carboxylate was purified via SFC using a Chiralcel OX 2×25 cm, 5 μm column with a mobile phase of 10% IPA using a flowrate of 120 mL/min to generate tert-butyl(S)-3′,7′-dimethyl-6′,7′-dihydrospiro[piperidine-4,4′-pyrazolo[5,1-c][1,4]oxazine]-1-carboxylate, 1^{st} eluting isomer (m/z (ESI): 321.6 (M+H)⁺) and tert-butyl (R)-3′,7′-dimethyl-6′,7′-dihydrospiro[piperidine-4,4′-pyrazolo[5,1-c][1,4]oxazine]-1-carboxylate, 2ªd eluting isomer (m/z (ESI): 321.6 (M+H)⁺). The stereochemistry was arbitrarily assigned.

Step 4: (S)-3′,7′-Dimethyl-6′,7-dihydrospiro[piperidine-4,4′-pyrazolo[5,1-c][1,4]oxazine], Intermediate B33-1. To a solution of the 1^{st} eluting isomer from step 3, (280 mg, 0.87 mmol) in DCM (5.0 mL) was added TfA (0.33 mL, 4.4 mmol), and the reaction mixture was stirred at rt for 30 min. The reaction was then concentrated to give Intermediate B33-1, as a TFA salt. m/s (ESI): 222.2 (M+H)⁺. The same step was performed with the 2^{nd} eluting isomer to provide Intermediate B33-2, as a TFA salt. m/z (ESI): 222.2 (M+H)⁺

### Linker Intermediates

### tert-Butyl 4-((2R,3R)-(-2-methylazetidin-3-yl)piperazine-1-carboxylate (Intermediate D-1)

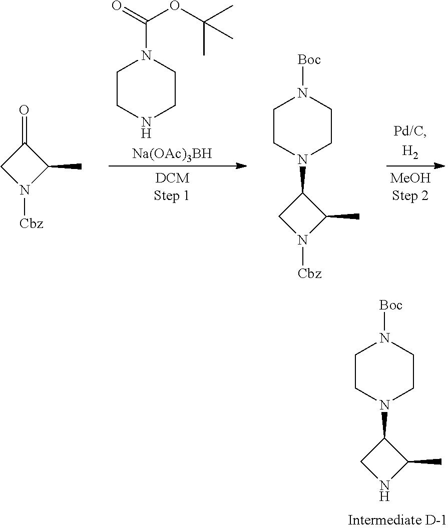

Step 1: tert-Butyl 4-((2R,3R)-1-((benzyloxy) carbonyl)-2-methylazetidin-3-yl)piperazine-1-carboxylate. To a mixture of benzyl (R)-2-methyl-3-oxoazetidine-1-carboxylate (250 g, 1140 mmol) and tert-butyl piperazine-1-carboxylate (319 g, 1710 mmol) in DCM (7.5 l) at 0° C. was added NaBH(OAc)₃ (725 g, 3.42 mol) and the reaction mixture was stirred at rt for 3 h. The reaction mixture was quenched with H₂O (3 L) and extracted with DCM (3 L). The organic extract was washed with brine (1 L), dried over Na₂SO₄, filtered, concentrated, and purified by silica gel chromatography, eluting with a gradient of 15% to 25% EtOAc in hexanes to give tert-butyl 4-((2R,3R)-1-((benzyloxy) carbonyl)-2-methylazetidin-3-yl)piperazine-1-carboxylate. MS (ESI) m/z: 390.3 (M+H)⁺. ¹H NMR (400 MHz, DMSO-d₆) δ 7.2-7.5 (m, 5H), 5.03 (s, 2H), 4.30 (s, 1H), 3.6-3.8 (m, 2H), 3.33 (br s, 4H), 3.12 (q, 1H, J=7.2 Hz), 2.17 (d, 4H, J=5.2 Hz), 1.39 (s, 9H), 1.32 (d, 3H, J=6.4 Hz).

Step 2: tert-Butyl 4-((2R,3R)-2-methylazetidin-3-yl)piperazine-1-carboxylate, Intermediate D-1. To a solution of tert-butyl 4-((2R,3R)-1-((benzyloxy)carbonyl)-2-methylazetidin-3-yl)piperazine-1-carboxylate (100 g, 2.57 mol) in MeOH (1 L) was added 10% Pd/C (38.3 g) and the reaction mixture was stirred under H₂ atmosphere (14 psi) at rt for 48 h. The reaction mixture was filtered over a pad of celite and washed with MeOH. The filtrate was concentrated to give Intermediate D-1. MS (ESI) m/z: 256.2 (M+H)⁺. ¹H NMR (400 MHz, DMSO-d₆) δ 3.5-3.7 (m, 1H), 3.2-3.4 (m, 6H), 2.99 (q, 1H, J=7.4 Hz), 2.0-2.1 (m, 4H), 1.39 (s, 9H), 1.23 (d, 3H, J=6.5 Hz).

Additional linker intermediates contemplated in the present disclosure include those listed in the table below. The preparation of said intermediates is described in International Application Nos. PCT/US2023/034535 and PCT/US2023/034537, each of which was filed on Oct. 5, 2023, and each of which is hereby incorporated by reference in its entirety.

TABLE 1-7

| Int. No. | Chemical Structure | Name |
|---|---|---|
| D-2 | 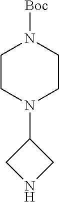 [SMILES: CC(C)(C)OC(=O)N1CCN(C2CNC2)CC1] | tert-butyl 4-(azetidin-3-yl)- piperazine-1-carboxylate |
| D-3 | 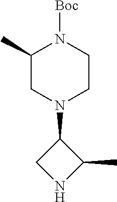 | tert-butyl (R)-2-methyl-4- ((2R,3R)-2-methylazetidin-3- yl)piperazine-1-carboxylate |
| D-4 | 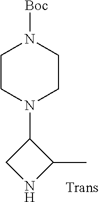 [SMILES: CC1NCC1N1CCN(C(=O)OC(C)(C)C)CC1] | tert-butyl 4-(2-methylazetidin- 3-yl)piperazine-1-carboxylate |
| D-5 | 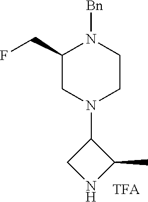 [SMILES: C[C@H]1NCC1N1CCN(Cc2ccccc2)[C@H](CF)C1] | (25)-1-benzyl-2-(fluoromethyl)- 4-((2R)-2-methyllazetidin-3-yl)- piperazine trifluoroacetate |
| D-6 | 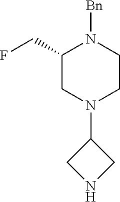 [SMILES: FC[C@H]1CN(C2CNC2)CCN1Cc1ccccc1] | (R)-4-(azetidin-3-yl)-1-benzyl- 2-(fluoromethyl)piperazine |
| D-7 | 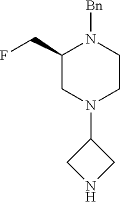 [SMILES: FC[C@@H]1CN(C2CNC2)CCN1Cc1ccccc1] | (S)-4-(azetidin-3-yl)-1-benzyl- 2-(fluoromethyl)piperazine |
| D-8 | 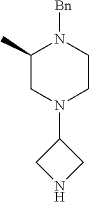 [SMILES: C[C@@H]1CN(C2CNC2)CCN1Cc1ccccc1] | tert-Butyl (R)-4-(azetidin-3- yl)-2-methylpiperazine-1- carboxylate |
| D-9 | 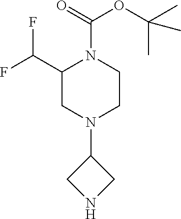 [SMILES: CC(C)(C)OC(=O)N1CCN(C2CNC2)CC1C(F)F] | tert-butyl 4-(azetidin-3-yl)-2- (difluoromethyl)piperazine-1- carboxylate |
| D-10 | 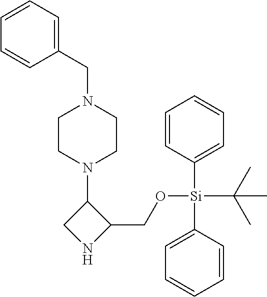 [SMILES: CC(C)(C)[Si](OCC1NCC1N1CCN(Cc2ccccc2)CC1)(c1ccccc1)c1ccccc1] | 1-benzyl-4-(2-(((tert-butyl- diphenylsilyl)oxy)methyl)- azetidin-3-yl)piperazine |

### Pyridine/Pyrimidine Core

### tert-Butyl 4-((2R,3R)-1-(6-chloro-2-(trifluoromethyl)pyrimidin-4-yl)-2-methylazetidin-3-yl)piperazine-1-carboxylate (Intermediate E-1)

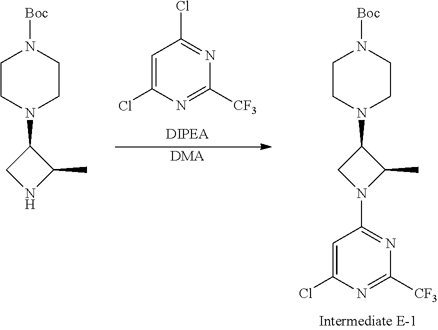

To a mixture of tert-butyl 4-((2R,3R)-2-methylazetidin-3-yl)piperazine-1-carboxylate (4.5 g, 17.6 mmol) and 4,6-dichloro-2-(trifluoromethyl)pyrimidine (4.21 g, 19.4 mmol) in DMA (45 mL) was added DIPEA (9.23 mL, 52.9 mmol) and the reaction mixture was stirred at rt for 3 h. The reaction mixture was quenched with H₂O (200 mL) and extracted with EtOAc (100 mL). The organic extract was washed with brine (60 mL), dried over Na₂SO₄, filtered, and concentrated. The crude residue was purified by silica gel chromatography, eluting with a gradient of 15% to 20% EtOAc in hexanes to give Intermediate E-1. MS (ESI) m/z: 436.1 (M+H)⁺. ¹H NMR (400 MHz, DMSO-d₆) δ 6.7-6.9 (m, 1H), 4.61 (q, J=5.4 Hz, 1H), 4.09 (t, J=8.8 Hz, 1H), 3.99 (t, J=8.5 Hz, 1H), 3.3-3.4 (m, 4H), 2.23 (d, J=8.5 Hz, 4H), 1.3-1.5 (m, 12H). ¹H NMR (377 MHz, DMSO-d₆) δ −70.20 (d, J=37.8 Hz, 3F).

The intermediates in the table below were prepared in a fashion similar to that was described above.

TABLE 1-8

| | | | MS (ESI) |
|---|---|---|---|
| Int. No. | Chemical Structure | Name | m/z: (M + H)⁺ |
| E-2 | 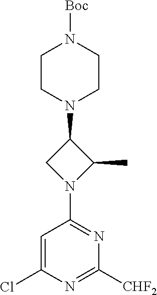 [SMILES: C[C@@H]1[C@H](N2CCN(C(=O)OC(C)(C)C)CC2)CN1c1cc(Cl)nc(C(F)F)n1] | tert-butyl 4-((2R.3R)-1-(6- chloro-2-(difluoromethyl)- pyrimidin-4-yl)-2-methyl- azetidin-3-yl)piperazine-1- carboxylate | 418.2 |
| E-3 | 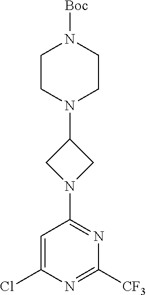 [SMILES: CC(C)(C)OC(=O)N1CCN(C2CN(c3cc(Cl)nc(C(F)(F)F)n3)C2)CC1] | tert-butyl 4-(1-(6-chloro-2- (trifluoromethyl)pyrimidin- 4-yl)azetidin-3-yl)piper- azine-1-carboxylate | 422.1 |

### tert-Butyl 4-(1-(6-chloro-3-cyano-2-(trifluoromethyl)pyridin-4-yl)azetidin-3-yl)piperazine-1-carboxylate (Intermediate E-4)

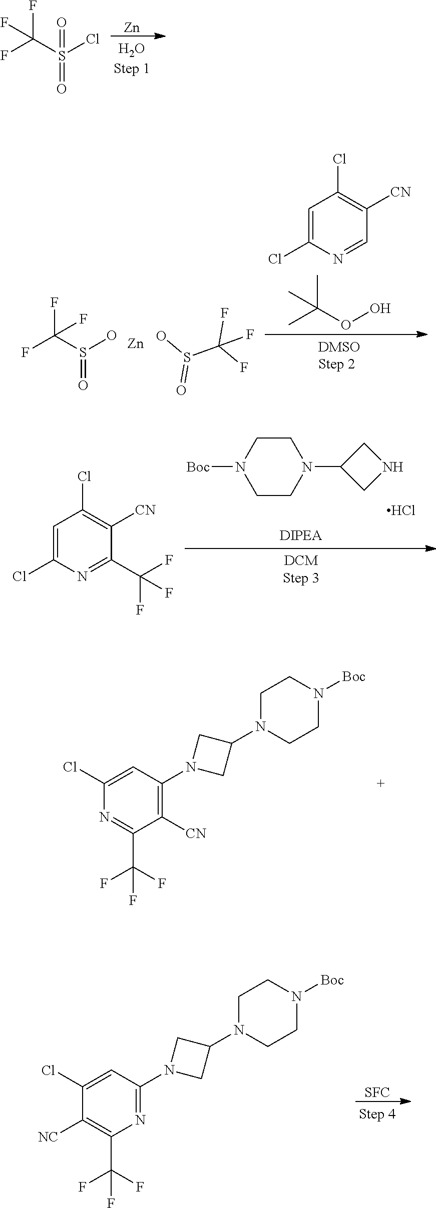

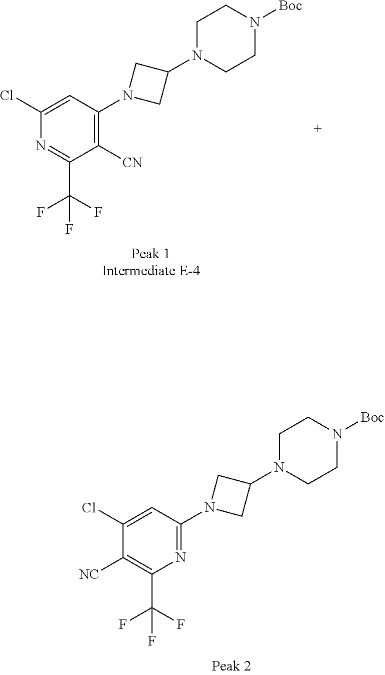 [SMILES: CC(C)(C)OC(=O)N1CCN(C2CN(c3cc(Cl)nc(C(F)(F)F)c3C#N)C2)CC1 ; CC(C)(C)OC(=O)N1CCN(C2CN(c3cc(Cl)c(C#N)c(C(F)(F)F)n3)C2)CC1]

Step 1: Bis(((trifluoromethyl)sulfinyl)oxy)zinc. To a suspension of zinc dust (76 g, 1.15 mol) in H₂O (160 mL) at 0° C. was added trifluoromethanesulfonyl chloride (30 g, 1.78 mol). The reaction mixture was stirred at rt for 2 h, filtered, and washed with H₂O (250 mL). The filtrate was concentrated under reduced pressure and co-evaporated with toluene (1500 mL) to give bis(((trifluoromethyl)sulfinyl)oxy)zinc.

Step 2:4,6-Dichloro-2-(trifluoromethyl)nicotinonitrile. To a solution of 4,6-dichloronicotonitrile (96 g, 0.56 mol) and bis(((trifluoromethyl)sulfinyl)oxy)zinc (368 g, 1.11 mol) in DMSo (2.7 L) at 0° C. was added 70% aq. solution of hydroperoxy-2-methylpropane (300 mL, 2.17 mol) over 30 min. The reaction mixture was stirred at rt for 16 h, cooled to 0° C., slowly quenched with 0.5 M aq. EDTA (600 mL), stirred for 5 min, and extracted with DCM (3×1 L). The combined organic extracts were washed with 0.5 M aqueous LiCl (1.1 L), dried over Na₂SO₄, filtered, and concentrated under reduced pressure. The residue was purified by silica gel chromatography, eluting with a gradient of 0% to 5% EtOAc in hexanes to give 4,6-dichloro-2-(trifluoromethyl)nicotinonitrile. ¹H NMR (400 MHz, DMSO-d₆) δ 8.62 (s, 1H).

Step 3: tert-Butyl 4-(1-(4-chloro-5-cyano-6-(trifluoromethyl)pyridin-2-yl)azetidin-3-yl)piperazine-1-carboxylate. To a solution of 4,6-dichloro-2-(trifluoromethyl)nicotinonitrile (83 g, 344 mmol) and tert-butyl 4-(azetidin-3-yl)piperazine-1-carboxylate hydrochloride (96 g, 344 mmol) in DCM (2 L) was added DIPEA (301 mL, 1.72 mol) and stirred at rt for 1 h. The reaction mixture was quenched with H₂O (1 L) and extracted with DCM (1 L). The organic layer was dried over Na₂SO₄, filtered, and concentrated under reduced pressure. The residue was purified by column chromatography, eluting with a gradient of 0% to 35% EtOAc:hexanes to give a mixture of tert-butyl 4-(1-(4-chloro-5-cyano-6-(trifluoromethyl)pyridin-2-yl)azetidin-3-yl)piperazine-1-carboxylate and its regioisomer. MS (ESI) m/z: 390.1 (M−tBu)⁺.

Step 4: tert-Butyl 4-(1-(6-chloro-3-cyano-2-(trifluoromethyl)pyridin-4-yl)azetidin-3-yl)piperazine-1-carboxylate, Intermediate E-4. The above isomers were separated by prep SFC using a Chiralcel AD-H column with a mobile phase of 85.5% liquid CO₂ and 9.5% MeOH and a flow rate of 190 mL/min. The 1^{st} eluting peak was assigned as Intermediate E-4. MS (ESI) m/z: 390.1 (M−tBu)⁺. ¹H NMR (400 MHz, DMSO-d₆) δ 6.87 (s, 1H), 4.2-4.6 (m, 4H), 3.3-3.5 (m, 5H), 2.3-2.4 (m, 4H), 1.48 (s, 9H). ¹⁹F NMR (376 MHz, DMSO-d₆) δ −65.25 (s, 3F). The 2^{nd} eluting peak was assigned as tert-butyl 4-(1-(4-chloro-5-cyano-6-(trifluoromethyl)pyridin-2-yl)azetidin-3-yl)piperazine-1-carboxylate. MS (ESI) m/z: 390.1 (M−tBu)⁺. ¹H NMR (400 MHz, DMSO-d₆) δ 7.00 (s, 1H), 4.19 (br s, 2H), 4.0 (br s, 2H) 3.3-3.5 (m, 5H), 2.33 (b s, 4H), 1.40 (s, 9H).

The below example was prepared in a manner similar to that described above.

TABLE 1-9

| | | | MS (ESI) |
|---|---|---|---|
| Int. No. | Chemical Structure | Name | m/z: (M + H)⁺ |
| E-5 | 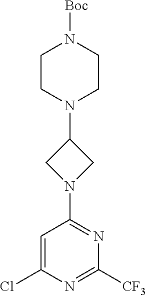 [SMILES: CC(C)(C)OC(=O)N1CCN(C2CN(c3cc(Cl)nc(C(F)(F)F)n3)C2)CC1] | tert-butyl 4-((2R.3R)-1- (6-chloro-3-cyano-2- (trifluoromethyl)pyridin- 4-yl)-2-methylazetidin- 3-yl)piperazine-1-carbox- ylate | 459.6 |

### tert-Butyl 4-(1-(6-chloro-2-(difluoromethyl)-3-methylpyridin-4-yl)azetidin-3-yl)piperazine-1-carboxylate (Intermediate E-6)

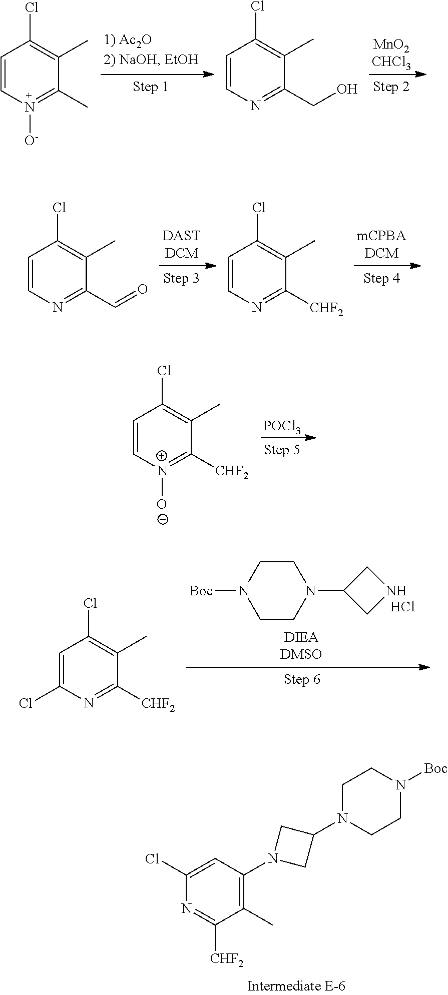

Step 1: (4-Chloro-3-methyl-2-pyridyl)methanol. A mixture of 4-chloro-2,3-dimethyl-1-oxido-pyridin-1-ium (95 g, 602 mmol) in Ac₂O (850 mL, 9.04 mol) was degassed and purged with N₂ (3 times), then the mixture was stirred at 110° C. for 2 h. The reaction mixture was concentrated under reduced pressure. The residue was diluted with H₂O (100 mL) and extracted with EtOAc (100 mL×3). The combined organic extracts were dried over Na₂SO₄, filtered, and concentrated under reduced pressure to give a residue. To the residue was added EtOH (1 L) and NaOH (26.5 g, 663 mmol), then the mixture was stirred at 80° C. for 2 h. The reaction mixture was concentrated under reduced pressure. The residue was diluted with H₂O (1 L) and extracted with EtOAc (500 mL×5). The combined organic extracts were dried over NazSO, filtered, and concentrated under reduced pressure. The residue was purified by silica gel chromatography, eluting with a gradient of 3% to 50% EtOAc in petroleum ether to afford (4-chloro-3-methyl-2-pyridyl)methanol. MS (ESI) m/z: 158.1 (M+H)⁺.

Step 2: 4-Chloro-3-methyl-pyridine-2-carbaldehyde. To a solution of (4-chloro-3-methyl-2-pyridyl)methanol (50 g, 317 mmol) in CHCl₃ (2 L) was added MnO₂ (276 g, 3.2 mol). The mixture was stirred at 70° C. for 12 h. The reaction mixture was filtered and concentrated under reduced pressure to give 4-chloro-3-methyl-pyridine-2-carbaldehyde, which was used without further purification. ¹H NMR (400 MHz, CDCl₃) δ 10.17 (s, 1H), 8.56 (d, J=4.8 Hz, 1H), 7.51 (d, J=4.8 Hz, 1H), 2.73 (s, 3H).

Step 3: 4-Chloro-2-(difluoromethyl)-3-methyl-pyridine. To a solution of 4-chloro-3-methyl-pyridine-2-carbaldehyde (30 g, 193 mmol) in DCM (300 mL) was added DAST (76.4 mL, 578 mmol) at 0° C. The mixture was stirred at 0° C. for 0.5 h. The reaction mixture was quenched by H₂O (500 mL) at 0° C. and extracted with DCM (300 mL×3). The combined organic extracts were dried over Na₂SO₄, filtered, and concentrated under reduced pressure to give 4-chloro-2-(difluoromethyl)-3-methyl-pyridine which was used without further purification. ¹H NMR (400 MHz, CDCl₃) δ 8.35 (d, J=4.8 Hz, 1H), 7.42 (d, J=5.2 Hz, 1H), 6.71 (t, J=54.4 Hz, 1H), 2.55 (s, 3H).

Step 4: 4-Chloro-2-(difluoromethyl)-3-methyl-1-oxido-pyridin-1-ium. To a solution of 4-chloro-2-(difluoromethyl)-3-methyl-pyridine (20 g, 113 mmol) in DCM (300 mL) was added m-CPBA (38.9 g, 180.2 mmol, 80%). The mixture was stirred at 40° C. for 12 h. The reaction mixture was diluted with H₂O (500 mL), extracted with DCM (500 mL×3). The combined organic extracts were dried over Na₂SO₄, filtered, and concentrated under reduced pressure to give 4-chloro-2-(difluoromethyl)-3-methyl-1-oxido-pyridin-1-ium. ¹H NMR (400 MHz, CDCl₃) δ 8.07 (d, J=6.8 Hz, 1H), 7.69 (t, J=52.8 Hz, 1H), 7.35 (d, J=6.8 Hz, 1H), 2.55 (s, 3H).

Step 5: 4,6-Dichloro-2-(difluoromethyl)-3-methyl-pyridine. A mixture of 4-chloro-2-(difluoromethyl)-3-methyl-1-oxido-pyridin-1-ium (40 g, 207 mmol) in POCl₃ (200 mL) was degassed and purged with N₂ 3 times, and the mixture was stirred at 100° C. for 12 h under a N₂ atmosphere. The reaction mixture was concentrated under reduced pressure to give a residue. The residue was suspended in H₂O (500 mL) and extracted with EtOAc (500 mL×3). The combined organic extracts were dried over Na₂SO₄, filtered, and concentrated under reduced pressure. The residue was purified by silica gel chromatography, eluting with a gradient of 2% to 100% EtOAc in petroleum ether to give 4,6-dichloro-2-(difluoromethyl)-3-methyl-pyridine. ¹H NMR (400 MHz, CDCl₃) δ 7.49 (s, 1H). 6.64 (t, J=54.0 Hz, 1H), 2.52 (t, J=1.6 Hz, 3H).

Step 6: tert-Butyl 4-(1-(4-chloro-6-(difluoromethyl)-5-methylpyridin-2-yl)azetidin-3-yl) piperazine-1-carboxylate, Intermediate E-6. To a solution of 4, 6-dichloro-2-(difluoromethyl)-3-methylpyridine (19 g, 90 mmol) in DMSO (300 mL) was added tert-butyl 4-(azetidin-3-yl) piperazine-1-carboxylate hydrochloride (25 g, 90 mmol) and DIPEA (34 mL, 193 mmol). The mixture was stirred at 80° C. for 12 h. The reaction mixture was diluted with H₂O (500 mL) and extracted with EtOAc (300 mL×3). The combined organic extracts were dried over Na₂SO₄, filtered, and concentrated under reduced pressure. The residue was purified by silica gel chromatography, eluting with a gradient of 0% to 17% EtOAc in petroleum ether to give tert-butyl 4-(1-(6-chloro-2-(difluoromethyl)-3-methylpyridin-4-yl)azetidin-3-yl)piperazine-1-carboxylate and Intermediate E-6. MS (ESI) m/z: 417.1 (M+H)⁺. ¹H NMR (400 MHz, Methanol-d₄) δ 6.55 (t, J=54.4 Hz, 1H), 6.31 (s, 1H), 4.20-4.24 (m, 2H), 3.95-4.01 (m, 2H), 3.47 (s, 4H), 3.20-3.30 (m, 1H), 2.34 (s, 4H), 2.29 (s, 3H), 1.47 (s, 9H).

### tert-Butyl 4-((2R,3R)-1-(6-chloro-2-(difluoromethyl)-3-methylpyridin-4-yl)-2-methylazetidin-3-yl) piperazine-1-carboxylate (Intermediate E-7)

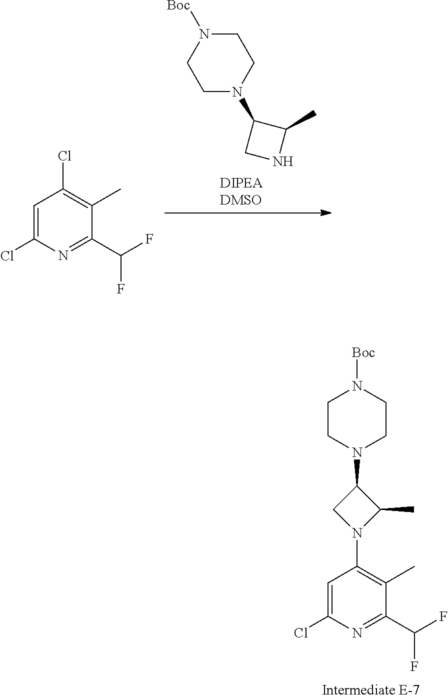 [SMILES: Cc1c(Cl)cc(Cl)nc1C(F)F ; C[C@H]1NC[C@H]1N1CCN(C(=O)OC(C)(C)C)CC1 ; Cc1c(N2C[C@@H](N3CCN(C(=O)OC(C)(C)C)CC3)[C@H]2C)cc(Cl)nc1C(F)F]

To a solution of 4,6-dichloro-2-(difluoromethyl)-3-methylpyridine (6.15 g, 29.0 mmol) in DMSO (200 mL) was added tert-butyl 4-((2R,3R)-2-methylazetidin-3-yl)piperazine-1-carboxylate (7.4 g, 29 mmol) and DIPEA (11 mL, 62 mmol). The mixture was stirred at 90° C. for 12 h. The reaction mixture was diluted with H₂O (400 mL) and extracted with EtOAc (100 mL×3). The combined organic extracts were dried over Na₂SO₄, filtered, and concentrated under reduced pressure. The residue was purified by silica gel chromatography (petroleum ether/EtOAc=30:1 to 1:1) to give tert-butyl 4-((2R,3R)-1-(6-chloro-2-(difluoromethyl)-3-methylpyridin-4-yl)-2-methylazetidin-3-yl) piperazine-1-carboxylate and Intermediate E-7. MS (ESI) m/z: 431.1 (M+H)⁺, ¹H NMR (400 MHz, CDCl₃) δ 6.56 (t, J=14.4 Hz, 1H), 6.39 (s, 1H), 4.50-4.65 (m, 1H), 3.75-3.85 (m, 1H), 3.85-3.95 (m, 1H), 3.45 (s, 4H), 3.30-3.38 (m, 1H), 2.28 (s, 4H), 2.24 (m, 3H), 1.47 (s, 9H), 1.21 (d, J=6.4 Hz, 3H).

### tert-Butyl 4-(1-(6-chloro-2-(trifluoromethyl)pyrimidin-4-yl)-3-methylazetidin-3-yl)piperazine-1-carboxylate (Intermediate E-8)

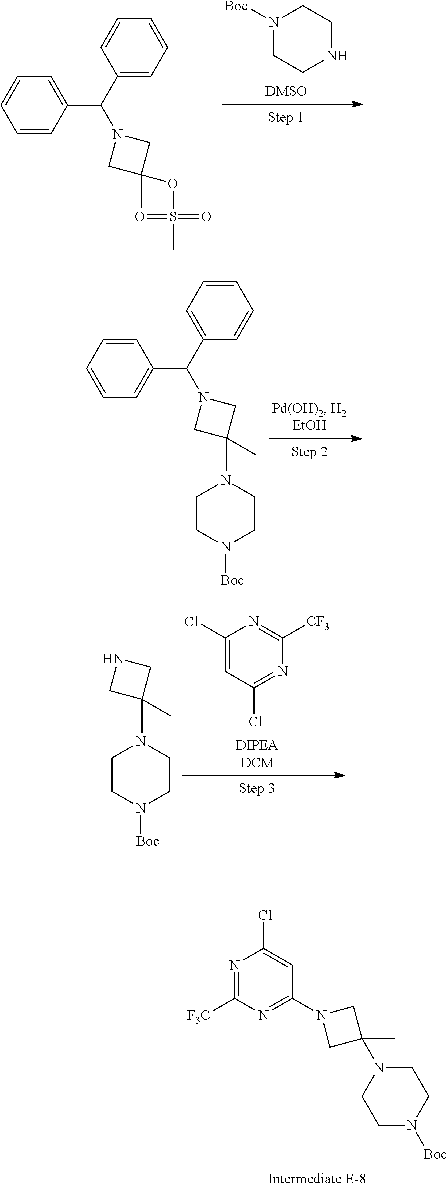

Step 1: tert-Butyl 4-(1-benzhydryl-3-methylazetidin-3-yl)piperazine-1-carboxylate. A glass vial was charged with 1-benzhydryl-3-methylazetidin-3-yl methanesulfonate (165 mg, 0.50 mmol) and tert-butyl piperazine-1-carboxylate (278 mg, 1.49 mmol) in DMSO (2 mL). The reaction mixture was shaken in a heat block at 120° C. for 18 h. It was then cooled to rt, diluted with 3 mL of DCM, and washed with 5% aq. LiCl (2×15 mL). The organic layer was taken and the solvent was removed under vacuum. The residue was purified by silica gel chromatography, eluting with a gradient of 0% to 100% EtOAc in heptane to provide tert-butyl 4-(1-benzhydryl-3-methylazetidin-3-yl)piperazine-1-carboxylate. MS (ESI) m/z: 422 (M+H)⁺.

Step 2: tert-Butyl 4-(3-methylazetidin-3-yl)piperazine-1-carboxylate. A solution of tert-butyl 4-(1-benzhydryl-3-methylazetidin-3-yl)piperazine-1-carboxylate (80 mg, 0.19 mmol) and Pd(OH)₂ (20 wt %, 110 mg, 0.157 mmol) in EtOH (8 mL) was subjected to 3 vacuum/H₂ cycles. The reaction was stirred at rt overnight, then filtered through celite. Pd(OH)₂ (20 wt %, 110 mg, 0.157 mmol) was added, and the mixture subjected to 3 vacuum/H₂ cycles. The reaction was stirred at rt for 3 h. then filtered through celite and evaporated to give tert-butyl 4-(3-methylazetidin-3-yl)piperazine-1-carboxylate. MS (ESI) m/z: 256 (M+H)⁺. The crude mixture was used in the next step without purification.

Step 3: tert-Butyl 4-(1-(6-chloro-2-(trifluoromethyl)pyrimidin-4-yl)-3-methylazetidin-3-yl)piperazine-1-carboxylate, Intermediate E-8. tert-Butyl 4-(3-methylazetidin-3-yl)piperazine-1-carboxylate (80 mg, 0.16 mmol) and DIPEA (0.082 mL, 0.47 mmol) in DCM (3 mL) were mixed together followed by 4,6-dichloro-2-(trifluoromethyl)pyrimidine (34.0 mg, 0.16 mmol). The reaction was stirred at rt for 4 h. It was then purified by silica gel chromatography, eluting with a gradient of 5% to 100% EtOAc in heptane to provide Intermediate E-8. MS (ESI) m/z: 436 (M+H)⁺. ¹H NMR (300 MHz, MeOH-d₄) δ 6.61 (s, 1H) 3.94-4.11 (m, 2H) 3.77-3.93 (m, 2H) 3.40-3.53 (m, 4H) 2.47 (br t, J=4.71 Hz, 4H) 1.47 (s, 9H) 1.37 (s, 3H).

### Section 2: Synthesis of Example Compounds

### Example 3-001: 1-(4-((2R,3R)-1-(6-(4H-Spiro[furo[3,4-d]thiazole-6,4′-piperidin]-1′-yl)-2-(trifluoromethyl)pyrimidin-4-yl)-2-methylazetidin-3-yl)piperazin-1-yl) prop-2-en-1-one

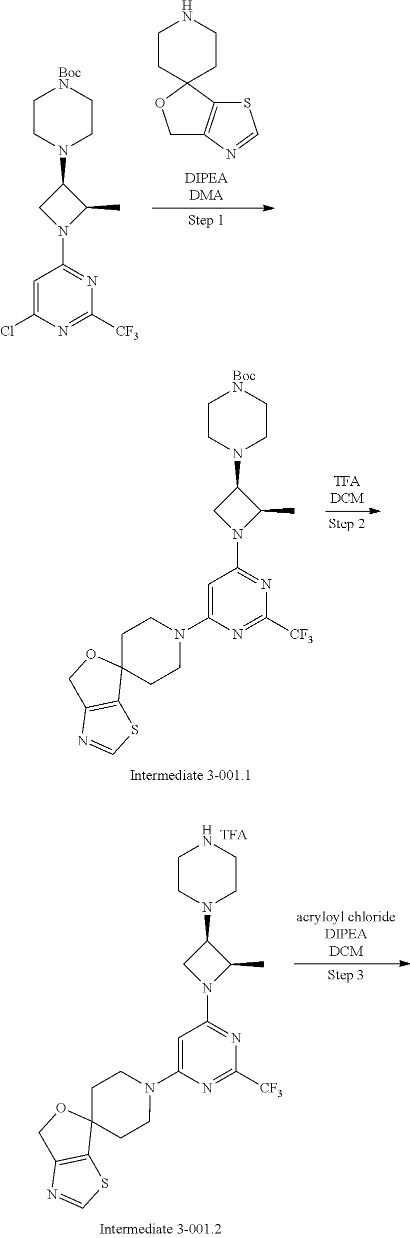 [SMILES: C[C@@H]1[C@H](N2CCN(C(=O)OC(C)(C)C)CC2)CN1c1cc(Cl)nc(C(F)(F)F)n1 ; c1nc2c(s1)C1(CCNCC1)OC2 ; C[C@@H]1[C@H](N2CCN(C(=O)OC(C)(C)C)CC2)CN1c1cc(N2CCC3(CC2)OCc2ncsc23)nc(C(F)(F)F)n1 ; C[C@@H]1[C@H](N2CCNCC2)CN1c1cc(N2CCC3(CC2)OCc2ncsc23)nc(C(F)(F)F)n1]

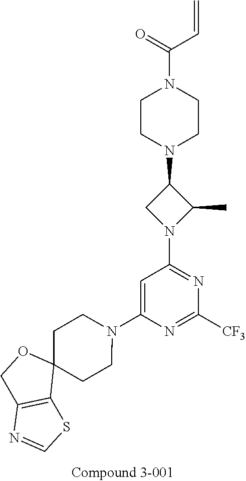 [SMILES: C=CC(=O)N1CCN([C@@H]2CN(c3cc(N4CCC5(CC4)OCc4ncsc45)nc(C(F)(F)F)n3)[C@@H]2C)CC1]

Step 1: tert-Butyl 4-((2R,3R)-1-(6-(4H-spiro[furo[3,4-d]thiazole-6,4′-piperidin]-1′-yl)-2-(trifluoromethyl)pyrimidin-4-yl)-2-methylazetidin-3-yl)piperazine-1-carboxylate, Intermediate 3-001.1. To a solution of tert-butyl 4-((2R,3R)-1-(6-chloro-2-(trifluoromethyl)pyrimidin-4-yl)-2-methylazetidin-3-yl)piperazine-1-carboxylate (500 mg, 1.15 mmol) (Intermediate B-2) and 4H-spiro[furo[3,4-d]thiazole-6,4′-piperidine] (248 mg, 1.27 mmol) (Intermediate B-4) in DMA (10 mL) was added DIPEA (1.6 mL, 9.2 mmol). The mixture was stirred at 90° C. for 12 h. The reaction mixture was quenched by addition of 30 mL H₂O at 10° C. and extracted with EtOAc (50 mL×3). The combined organic extracts were washed with brine (20 mL×2), dried over Na₂SO₄, filtered, and concentrated under reduced pressure. The residue was purified by silica gel chromatography, eluting with a gradient of 0% to 33% petroleum ether in EtOAc to provide Intermediate 3-001.1. MS (ESI) m/z: 596.4 (M+H)⁺.

Step 2: 1′-(6-((2R,3R)-2-Methyl-3-(piperazin-1-yl)azetidin-1-yl)-2-(trifluoromethyl)pyrimidin-4-yl)-4H-spiro[furo[3,4-d]thiazole-6,4′-piperidine] (TFA salt), Intermediate 3-001.2. To a solution of Intermediate 3-001.1 (265 mg, 0.45 mmol) in DCM (5 mL) was added TFA (1 mL, 13 mmol). The mixture was stirred at 20° C. for 5 h. The reaction mixture was concentrated under reduced pressure to give Intermediate 3-001.2, as a TFA salt.

Step 3: 1-(4-((2R,3R)-1-(6-(4H-Spiro[furo[3,4-d]thiazole-6,4′-piperidin]-1′-yl)-2-(trifluoromethyl)pyrimidin-4-yl)-2-methylazetidin-3-yl)piperazin-1-yl) prop-2-en-1-one, Compound 3-001. To a solution of Intermediate 3-001.2 TFA salt (300 mg, 0.51 mmol) and DIPEA (441 μL, 2.53 mmol) in DCM (2 mL) was added acryloyl chloride (33 μL, 0.40 mmol) dropwise at 0° C. The mixture was stirred at 0° C. for 0.5 h, concentrated under reduced pressure, and purified by pre-TLC, eluting with a mobile phase of 10% MeOH in EtOAc to give Compound 3-001. MS (ESI) m/z: 550.2 (M+H)⁺. ¹H NMR (400 MHz, DMSO-d₆) δ 9.09 (s, 1H), 6.90-6.70 (dd, J=16.8, 10.4 Hz, 1H), 6.15-6.05 (dd, J=16.8, 2.4 Hz, 1H), 5.75-5.60 (m, 2H), 5.00 (s, 2H), 4.55-4.45 (m, 1H), 4.00-3.90 (m, 1H), 3.90-3.80 (m, 3H), 3.75-3.45 (m, 6H), 3.30-3.20 (m, 1H), 2.28 (s, 4H), 2.00-1.75 (m, 4H), 1.39 (d, J=6.00 Hz, 3H). ¹⁹F NMR (377 MHz, DMSO-d₆) δ −70.22 (s, 3F).

### Example 3-002: 1-(4-((2R,3R)-1-(6-(6′,7-Dihydrospiro[piperidine-4,4′-pyrano[4,3-d]thiazol]-1-yl)-2-(trifluoromethyl)pyrimidin-4-yl)-2-methylazetidin-3-yl)piperazin-1-yl)prop-2-en-1-one

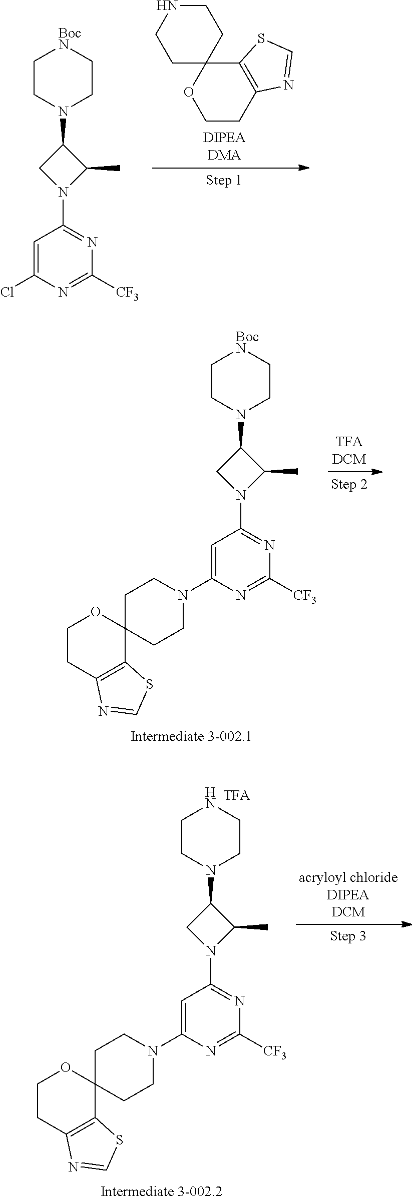 [SMILES: C[C@@H]1[C@H](N2CCN(C(=O)OC(C)(C)C)CC2)CN1c1cc(Cl)nc(C(F)(F)F)n1 ; c1nc2c(s1)C1(CCNCC1)OCC2 ; C[C@@H]1[C@H](N2CCN(C(=O)OC(C)(C)C)CC2)CN1c1cc(N2CCC3(CC2)OCCc2ncsc23)nc(C(F)(F)F)n1 ; C[C@@H]1[C@H](N2CCNCC2)CN1c1cc(N2CCC3(CC2)OCCc2ncsc23)nc(C(F)(F)F)n1]

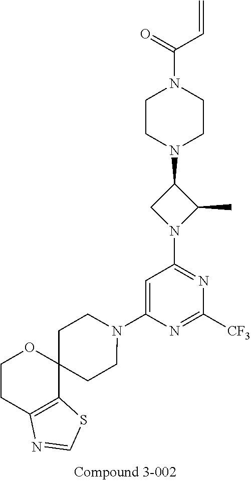 [SMILES: C=CC(=O)N1CCN([C@@H]2CN(c3cc(N4CCC5(CC4)OCCc4ncsc45)nc(C(F)(F)F)n3)[C@@H]2C)CC1]

Step 1: tert-Butyl 4-((2R,3R)-1-(6-(6′,7′-dihydrospiro[piperidine-4,4′-pyrano[4,3-d]thiazol]-1-yl)-2-(trifluoromethyl)pyrimidin-4-yl)-2-methylazetidin-3-yl)piperazine-1-carboxylate. Intermediate 3-002.1. To a solution of tert-butyl 4-((2R,3R)-1-(6-chloro-2-(trifluoromethyl)pyrimidin-4-yl)-2-methylazetidin-3-yl)piperazine-1-carboxylate (207 mg, 0.48 mmol) and 6′,7′-dihydrospiro[piperidine-4,4′-pyrano[4,3-d]thiazole] (100 mg, 0.48 mmol) (Intermediate B-8) in DMA (2 mL) was added DIPEA (415 μL, 2.38 mmol). The mixture was stirred at 90° C. for 12 h. The reaction mixture was quenched by addition of 30 mL H₂O, and then extracted with EtOAc (10 mL×3). The combined organic extracts were washed with brine (10 mL×2), dried over Na₂SO₄, filtered, and concentrated under reduced pressure. The residue was purified by prep-TLC, eluting with a mobile phase of EtOAc to give Intermediate 3-002.1.

Step 2:1-(6-((2R,3R)-2-Methyl-3-(piperazin-1-yl)azetidin-1-yl)-2-(trifluoromethyl)pyrimidin-4-yl)-6′,7′-dihydrospiro[piperidine-4,4′-pyrano[4,3-d]thiazole] (TFA salt), Intermediate 3-002.2. To a solution of Intermediate 3-002.1 (120 mg, 0.20 mmol) in DCM (2 mL) was added TFA (0.4 mL, 5.2 mmol). The mixture was stirred at 20° C. for 1 h. The reaction mixture was concentrated under reduced pressure to give Intermediate 3-002.2, as a TFA salt, that was taken to next step.

Step 3: 1-(4-((2R,3R)-1-(6-(6′,7′-Dihydrospiro[piperidine-4,4′-pyrano[4,3-d]thiazol]-1-yl)-2-(trifluoromethyl)pyrimidin-4-yl)-2-methylazetidin-3-yl)piperazin-1-yl)prop-2-en-1-one, Compound 3-002. To a solution of Intermediate 3-002.2 (TFA salt) (150 mg, 0.25 mmol) and DIPEA (216 μL, 1.23 mmol) in DCM (3 mL) was added acryloyl chloride (16 μL, 0.20 mmol) dropwise at 0° C. The mixture was stirred at 0° C. for 10 min. The reaction mixture was concentrated under reduced pressure to provide Compound 3-002. MS (ESI) m/z: 564.2 (M+H)⁺. ¹H NMR (400 MHz, DMSO-d₆) δ 8.98 (s, 1H), 6.85-6.75 (m, 1H), 6.15-6.05 (dd, J=16.8, 2.4 Hz, 1H), 5.70-5.60 (m, 2H), 4.50-4.40 (m, 1H), 4.22 (s, 2H), 4.01 (1, J=5.6 Hz, 2H), 3.90-3.76 (m, 2H), 3.72-3.50 (m, 4H), 3.24-3.10 (m, 4H). 2.83 (t, J=5.2 Hz, 2H), 2.40-2.20 (m, 4H), 2.08-1.95 (m, 2H), 1.82-1.65 (m, 2H), 1.39 (d, J=6.40 Hz, 3H). ¹⁹F NMR (377 MHz, DMSO-d₆) δ −70.21 (s).

### Example 3-003: 1-(4-((2R,3R)-1-(2-(Difluoromethyl)-6-((7R)-4,7-dimethyl-1-oxa-8-azaspiro[4.5]dec-3-cn-8-yl)pyrimidin-4-yl)-2-methylazetidin-3-yl) piperazin-1-yl) prop-2-on-1-one

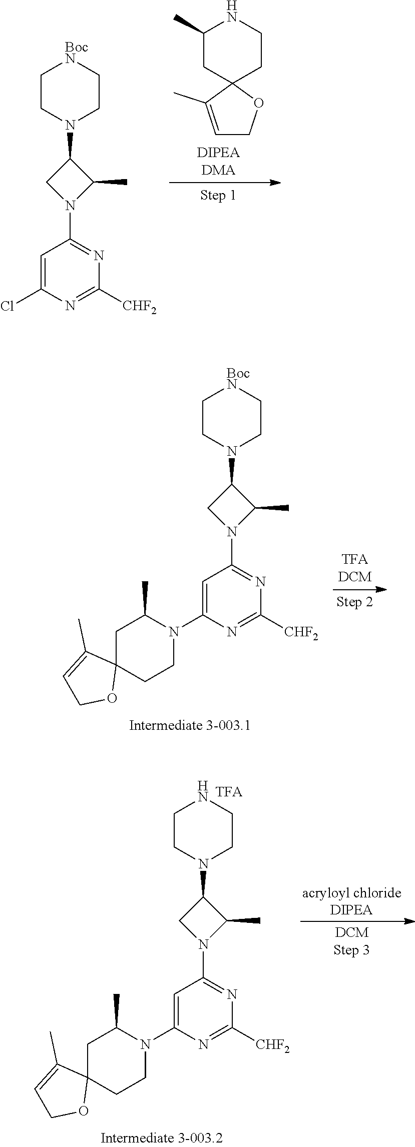 [SMILES: C[C@@H]1[C@H](N2CCN(C(=O)OC(C)(C)C)CC2)CN1c1cc(Cl)nc(C(F)F)n1 ; CC1=CCOC12CCN[C@H](C)C2 ; CC1=CCOC12CCN(c1cc(N3C[C@@H](N4CCN(C(=O)OC(C)(C)C)CC4)[C@H]3C)nc(C(F)F)n1)[C@H](C)C2 ; CC1=CCOC12CCN(c1cc(N3C[C@@H](N4CCNCC4)[C@H]3C)nc(C(F)F)n1)[C@H](C)C2]

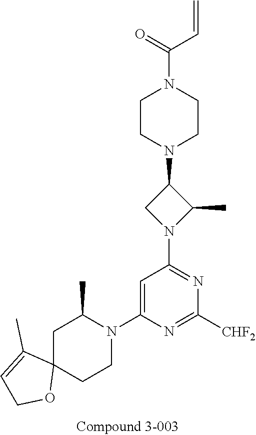 [SMILES: C=CC(=O)N1CCN([C@@H]2CN(c3cc(N4CCC5(C[C@H]4C)OCC=C5C)nc(C(F)F)n3)[C@@H]2C)CC1]

Step 1: tert-Butyl 4-((2R,3R)-1-(2-(difluoromethyl)-6-((7R)-4,7-dimethyl-1-oxa-8-azaspiro[4.5]dec-3-en-8-yl)pyrimidin-4-yl)-2-methylazetidin-3-yl)piperazine-1-carboxylate, Intermediate 3-003.1. (7R)-4,7-Dimethyl-1-oxa-8-azaspiro[4.5]dec-3-ene (250 mg, 1.5 mmol) (Intermediate B-2) and tert-butyl 4-((2R,3R)-1-(2-(difluoromethyl)-6-fluoropyrimidin-4-yl)-2-methylazetidin-3-yl)piperazine-1-carboxylate (300 mg, 0.7 mmol) in DMA (6 mL) were mixed under N₂ atm. followed by addition of DIPEA (1.30 mL, 7.5 mmol) at 0° C. The reaction mixture was stirred at 120° C. for 2 days. The reaction mixture was quenched with ice cold H₂O and extracted with EtOAc (2×25 mL). The combined organic extracts were dried over Na₂SO₄, filtered, and concentrated in vacuo to give the crude material, which was purified by chromatography eluting with a gradient of 0% to 20% EtOAc in hexanes, to provide Intermediate 3-003.1. MS (ESI) m/z: 549.3 (M+H)⁺.

Step 2: (7R)-8-(2-(Difluoromethyl)-6-((2R,3R)-2-methyl-3-(piperazin-1-yl)azetidin-1-yl)pyrimidin-4-yl)-4,7-dimethyl-1-oxa-8-azaspiro[4.5]dec-3-ene (TFA salt), Intermediate 3-003.2. To a rbf was added Intermediate 3-003.1 (350 mg, 0.64 mmol) in DCM (5 mL) under N₂ atm, followed by addition of TFA (250 μL, 3.2 mmol) at 0° C. The reaction mixture was stirred at rt for 3 h. The reaction mixture was concentrated in vacuo to give Intermediate 3-003.2 (TFA salt), which was used without further purification. MS (ESI) m/z: 449.3 (M+H)⁺.

Step 3: 1-(4-((2R,3R)-1-(2-(Difluoromethyl)-6-((7R)-4,7-dimethyl-1-oxa-8-azaspiro[4.5]dec-3-en-8-yl)pyrimidin-4-yl)-2-methylazetidin-3-yl)piperazin-1-yl) prop-2-en-1-one, Compound 3-003. Intermediate 3-003.2 (TFA salt) (270 mg, 0.60 mmol) in DCM (6 mL) under N₂ atm, was mixed with DIPEA (530 μL, 3.01 mmol) and acryloyl chloride (60 mg, 0.66 mmol) at 0° C. The reaction mixture was stirred at rt for 1 h. The reaction mixture was quenched with cold H₂O and extracted with DCM (2×30 mL). The combined organic extracts were dried over Na₂SO₄, filtered, and concentrated in vacuo to give crude product, which was purified by HPLC to afford Compound 3-003. MS (ESI) m/z: 503.2 (M+H)⁺. ¹H NMR (DMSO-d₆, 400 MHz) δ 6.80 (dd, J=16.7, 10.5 Hz, 1H), 6.40 (s, 1H), 6.11 (dd, J=16.7, 2.4 Hz, 1H), 5.69 (dd, J=10.4, 2.4 Hz, 1H), 5.59 (q, J=1.6 Hz, 1H), 5.46 (s, 2H), 4.46-4.52 (m, 2H), 3.91 (t, J=8.2 Hz, 1H), 3.84 (dd, J=8.8, 6.7 Hz, 1H), 3.58 (s, 2H), 3.23 (q, J=7.2 Hz, 2H), 3.14 (d, J=13.6 Hz, 2H), 2.29 (s. 4H), 1.86 (dd, J=13.9, 6.5 Hz, 1H), 1.60-1.73 (m, 4H), 1.36-1.50 (m,6H), 1.26 (d, J=6.9 Hz, 3H).

### Example 3-004: 4-(3-(4-Acryloylpiperazin-1-yl)azetidin-1-yl)-6-(2-methyl-3-oxospiro[isoindoline-1,4′-piperidin]-1′-yl)-2-(trifluoromethyl)nicotinonitrile

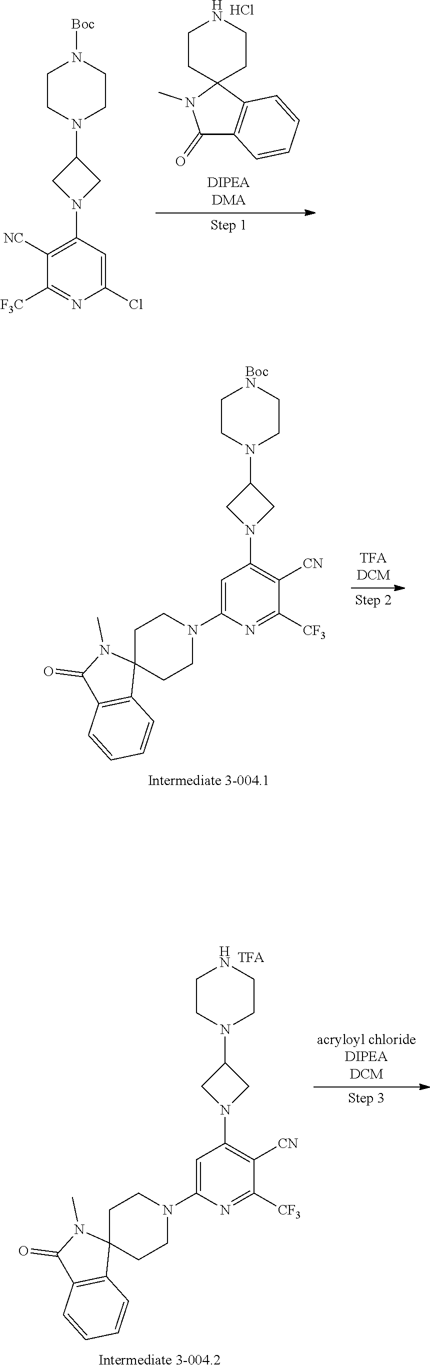 [SMILES: Cl ; CC(C)(C)OC(=O)N1CCN(C2CN(c3cc(Cl)nc(C(F)(F)F)c3C#N)C2)CC1 ; CN1C(=O)c2ccccc2C12CCNCC2 ; CN1C(=O)c2ccccc2C12CCN(c1cc(N3CC(N4CCN(C(=O)OC(C)(C)C)CC4)C3)c(C#N)c(C(F)(F)F)n1)CC2 ; CN1C(=O)c2ccccc2C12CCN(c1cc(N3CC(N4CCNCC4)C3)c(C#N)c(C(F)(F)F)n1)CC2]

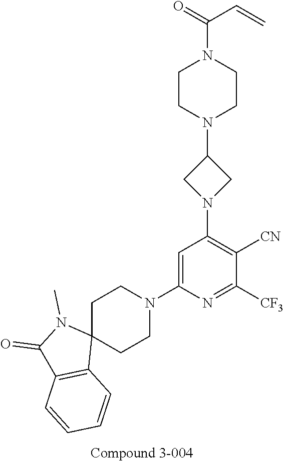 [SMILES: C=CC(=O)N1CCN(C2CN(c3cc(N4CCC5(CC4)c4ccccc4C(=O)N5C)nc(C(F)(F)F)c3C#N)C2)CC1]

Step 1: tert-Butyl 4-(1-(3-cyano-6-(2-methyl-3-oxospiro[isoindoline-1,4′-piperidin]-1′-yl)-2-(trifluoromethyl)pyridin-4-yl)azetidin-3-yl)piperazine-1-carboxylate, Intermediate 3-004.1. To a glass vial with a stir bar was added 2-methylspiro[isoindoline-1,4′-piperidin]-3-one hydrochloride (100 mg, 0.40 mmol) in DMA (2.0 mL), and DIPEA (690 μL, 4.0 mmol) at rt. The reaction mixture was stirred for 10 min at rt, and tert-butyl 4-(1-(6-chloro-3-cyano-2-(trifluoromethyl)pyridin-4-yl)azetidin-3-yl)piperazine-1-carboxylate (159 mg, 0.356 mmol) was added. The reaction mixture was then heated to 86° C. and stirred 24 h. The reaction mixture was quenched by sat. aq. NH₄Cl solution (2 mL). The organic phase was washed with H₂O (2 mL) and sat. aq. NH₄Cl solution (2 mL×2). The combined aqueous phases were extracted with 2 mL EtOAc. The combined organic phases were washed with brine (4 mL), dried over Na₂SO₄, and concentrated. The crude material was purified by silica gel chromatography eluting with a gradient of 1% to 6% MeOH in DCM to provide Intermediate 3-004.1. MS (ESI) m/z: 626.2 (M+H)⁺.

Step 2: 6-(2-Methyl-3-oxospiro[isoindoline-1,4′-piperidin]-1′-yl)-4-(3-(piperazin-1-yl)azetidin-1-yl)-2-(trifluoromethyl)nicotinonitrile, Intermediate 3-004.2. tert-Butyl 4-(1-(3-cyano-6-(2-methyl-3-oxospiro[isoindoline1,4′-piperidin]-1′-yl)-2-(trifluoromethyl)pyridin-4-yl)azetidin-3-yl)piperazine-1-carboxylate (175 mg, 0.28 mmol) and TFA (420 μL, 5.6 mmol) in DCM (3 mL) were stirred at rt for 1 h. The reaction mixture was concentrated under vacuum and the crude mixture containing Intermediate 3-004.2 was used directly in the next step.

Step 3: 4-(3-(4-Acryloylpiperazin-1-yl)azetidin-1-yl)-6-(2-methyl-3-oxospiro[isoindoline-1,4′-piperidin]-1′-yl)-2-(trifluoromethyl)nicotinonitrile, Compound 3-004. To the crude mixture from Step 2 was added 1.5 mL DCM followed by DIPEA (490 μL, 2.8 mmol) and acryloyl chloride (0.2 M in DCM, 1400 μL, 0.30 mmol) at rt, and stirred for 15 min. The reaction solution was quenched and washed by sat. aq. NH₄Cl solution (4 mL) following by brine. The organic phase was dried with Na₂SO₄ and concentrated. The crude product was purified by SFC (1-AA 30×150 mm column with a mobile phase of 25-45% MeOH and a flow rate: 100 mL/min), to provide Compound 3-004. MS (ESI) m/z: 580.0 (M+H)⁺. ¹H NMR (500 MHz, DMSO-d₆) δ 8.08 (d, J=7.7 Hz, 1H), 7.73 (d, J=7.5 Hz, 1H), 7.60 (t, J=7.5 Hz, 1H), 7.52-7.56 (m, 1H), 6.80 (dd, J=16.7, 10.5 Hz, 1H), 6.10 (dd, J=16.7, 2.4 Hz, 1H), 5.86 (s, 1H), 5.66-5.70 (m, 1H), 4.39-4.53 (m, 2H), 4.33 (br t, J=7.2 Hz, 2H), 4.09 (br dd, J=8.9, 5.0 Hz, 2H), 3.52-3.68 (m, 6H), 2.93 (s, 3H), 2.36 (br s, 3H), 2.22 (td, J=12.9, 5.0 Hz, 2H), 1.52 (br d, J=13.4 Hz, 2H).

Examples in the table below were prepared in a manner similar to that described above.

TABLE 2-1

| | | | MS (ESI) |
|---|---|---|---|
| Compound | | | m/z: |
| No. | Chemical Structure | Name | (M +H)⁺ |
| 3-005 | 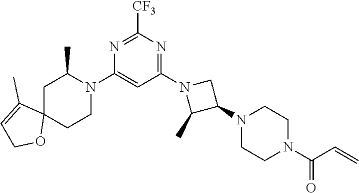 [SMILES: C=CC(=O)N1CCN([C@@H]2CN(c3cc(N4CCC5(C[C@H]4C)OCC=C5C)nc(C(F)(F)F)n3)[C@@H]2C)CC1] | 1-(4-((2R,3R)-1-(6-(4.7-dimethyl- 1-oxa-8-azaspiro[4.5]dec-3-en- 8-yl)-2-(trifluoromethyl)pyr- imidin-4-yl)-2-methylazetidin-3- yl)piperazin-1-yl)prop-2-en-1-one | 521.3 |
| 3-006 | 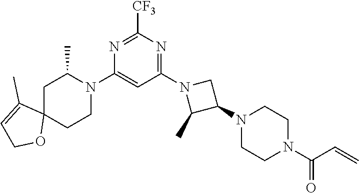 [SMILES: C=CC(=O)N1CCN([C@@H]2CN(c3cc(N4CCC5(C[C@@H]4C)OCC=C5C)nc(C(F)(F)F)n3)[C@@H]2C)CC1] | 1-(4-((2R,3R)-1-(6-(4,7-dimethyl- 1-oxa-8-azaspiro[4.5]dec-3-en-8- yl)-2-(trifluoromethyl)pyrimidin- 4-yl)-2-methylazetidin-3-yl)piper- azin-1-yl)prop-2-en-1-one | 521.2 |
| 3-007 | 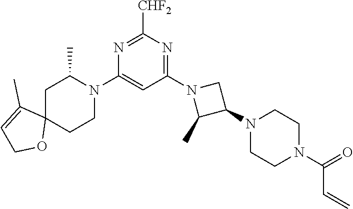 [SMILES: C=CC(=O)N1CCN([C@@H]2CN(c3cc(N4CCC5(C[C@@H]4C)OCC=C5C)nc(C(F)F)n3)[C@@H]2C)CC1] | 1-(4-((2R,3R)-1-(2-(difluorometh- yl)-6-(4.7-dimethyl-1-oxa-8-aza- spiro[4.5]dec-3-en-8-yl)pyrimidin- 4-yl)-2-methyl-azetidin-3-yl)piper- azin-1-yl)-prop-2-en-1-one | 503.2 |
| 3-008 | 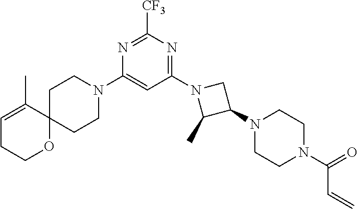 [SMILES: C=CC(=O)N1CCN([C@@H]2CN(c3cc(N4CCC5(CC4)OCCC=C5C)nc(C(F)(F)F)n3)[C@@H]2C)CC1] | 1-(4-((2R.3R)-2-methyl-1-(6-(5- methyl-1-oxa-9-azaspiro[5.5] undec-4-en-9-yl)-2-(trifluorometh- yl)pyrimidin-4-yl)azetidin-3-yl)- piperazin-1-yl)prop-2-en-1-one | 521.3 |
| 3-009 | 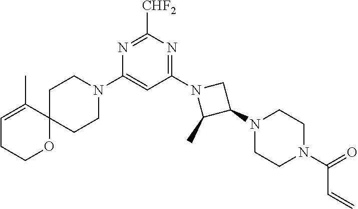 [SMILES: C=CC(=O)N1CCN([C@@H]2CN(c3cc(N4CCC5(CC4)OCCC=C5C)nc(C(F)F)n3)[C@@H]2C)CC1] | 1-(4-((2R,3R)-1-(2-(difluorometh- yl)-6-(5-methyl-1-oxa-9-azaspiro [5.5]undec-4-en-9-yl)-pyrimidin- 4-yl)-2-methylazetidin-3-yl)piper- azin-1-yl)prop-2-en-1-one | 503.2 |
| 3-010 | 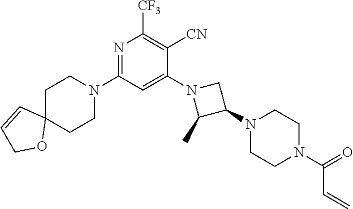 [SMILES: C=CC(=O)N1CCN([C@@H]2CN(c3cc(N4CCC5(C=CCO5)CC4)nc(C(F)(F)F)c3C#N)[C@@H]2C)CC1] | 4-((2R,3R)-3-(4-acryloylpiperazin- 1-yl)-2-methylazetidin-1-yl)-6-(1- oxa-8-azaspiro[4.5]dec-3-en-8-yl)- 2-(trifluoromethyl)nicotinonitrile | 517.2 |
| 3-011 | 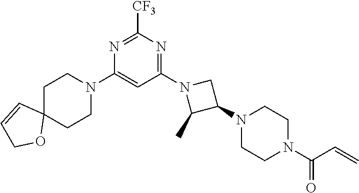 [SMILES: C=CC(=O)N1CCN([C@@H]2CN(c3cc(N4CCC5(C=CCO5)CC4)nc(C(F)(F)F)n3)[C@@H]2C)CC1] | 1-(4-((2R,3R)-1-(6-(1-oxa-8-aza- spiro[4.5]dec-3-en-8-yl)-2-(tri- fluoromethyl)pyrimidin-4-yl)-2- methylazetidin-3-yl)piperazin-1- yl)prop-2-en-1-one | 493.2 |
| 3-012 | 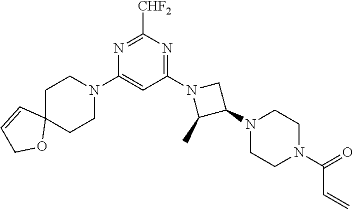 [SMILES: C=CC(=O)N1CCN([C@@H]2CN(c3cc(N4CCC5(C=CCO5)CC4)nc(C(F)F)n3)[C@@H]2C)CC1] | 1-(4-((2R,3R)-1-(2-(difluorometh- yl)-6-(1-oxa-8-azaspiro[4.5]dec-3- en-8-yl)pyrimidin-4-yl)-2-methyl- azetidin-3-yl)piperazin-1-yl)prop- 2-en-1-one | 475.2 |
| 3-013 | 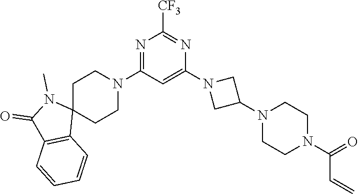 [SMILES: C=CC(=O)N1CCN(C2CN(c3cc(N4CCC5(CC4)c4ccccc4C(=O)N5C)nc(C(F)(F)F)n3)C2)CC1] | 1′-(6-(3-(4-acryloylpiperazin-1-yl- azetidin-1-yl)-2-(trifluoromethyl)- pyrimidin-4-yl)-2-methylspiro [isoindoline-1.4′-piperidin]-3-one | 556.0 |
| 3-014 | 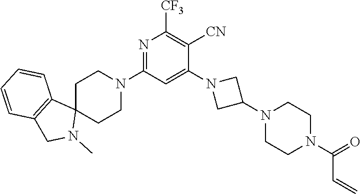 [SMILES: C=CC(=O)N1CCN(C2CN(c3cc(N4CCC5(CC4)c4ccccc4CN5C)nc(C(F)(F)F)c3C#N)C2)CC1] | 4-(3-(4-acryloylpiperazin-1-yl- azetidin-1-yl)-6-(2-methylspiro [isoindoline-1,4′-piperidin]-l′-yl)- 2-(trifluoromethyl)nicotinonitrile | 566.2 |
| 3-015 | 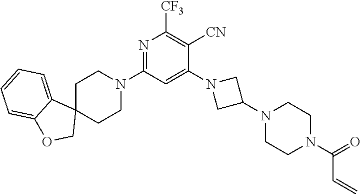 [SMILES: C=CC(=O)N1CCN(C2CN(c3cc(N4CCC5(CC4)COc4ccccc45)nc(C(F)(F)F)c3C#N)C2)CC1] | 4-(3-(4-acryloylpiperazin-1-yl- azetidin-1-yl)-6-(2H-spiro[benzo- furan-3,4′-piperidin]-1′-yl)-2- (trifluoromethyl)nicotinonitrile | 553.2 |
| 3-016 | 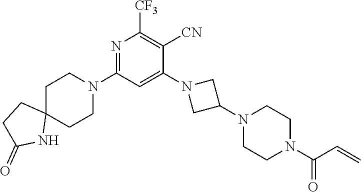 [SMILES: C=CC(=O)N1CCN(C2CN(c3cc(N4CCC5(CCC(=O)N5)CC4)nc(C(F)(F)F)c3C#N)C2)CC1] | 4-(3-(4-acryloylpiperazin-1-yl- azetidin-1-yl)-6-(2-oxo-1,8-diaza- spiro[4.5]decan-8-yl)-2-(trifluoro- methyl)nicotinonitrile | 518.2 |
| 3-017 | 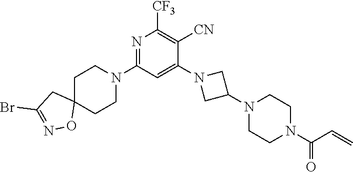 [SMILES: C=CC(=O)N1CCN(C2CN(c3cc(N4CCC5(CC4)CC(Br)=NO5)nc(C(F)(F)F)c3C#N)C2)CC1] | 4-(3-(4-acryloylpiperazin-1-yl)- azetidin-1-yl)-6-(3-bromo-1-oxa- 2.8-diazaspiro[4.5]dec-2-en-8-yl)- 2-(trifluoromethyl)nicotinonitrile | 582.19/ 584.19 |
| 3-018 | 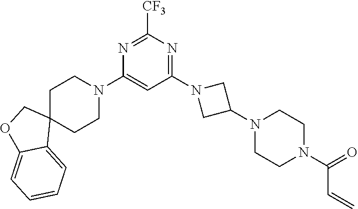 [SMILES: C=CC(=O)N1CCN(C2CN(c3cc(N4CCC5(CC4)COc4ccccc45)nc(C(F)(F)F)n3)C2)CC1] | 1-(4-(1-(6-(2H-spiro[benzofuran- 3,4′-piperidin]-1′-yl)-2-(trifluoro- methyl)pyrimidin-4-yl)azetidin-3- yl)piperazin-1-yl)prop-2-en-1-one | 529.2 |
| 3-019 | 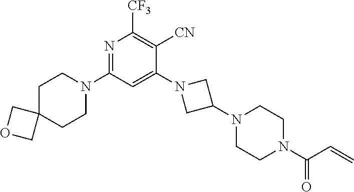 [SMILES: C=CC(=O)N1CCN(C2CN(c3cc(N4CCC5(CC4)COC5)nc(C(F)(F)F)c3C#N)C2)CC1] | 4-(3-(4-acryloylpiperazin-1-yl)- azetidin-1-yl)-6-(2-oxa-7-azaspiro [3.5]nonan-7-yl)-2-(trifluorometh- yl)nicotinonitrile | 491.2 |

### Example 3-020: 8-(6-((2R,3R)-3-(4-Acryloylpiperazin-1-yl)-2-methylazetidin-1-yl)-2-(trifluoromethyl)pyrimidin-4-yl)-1-methyl-1,8-diazaspiro[4.5]decan-2-one

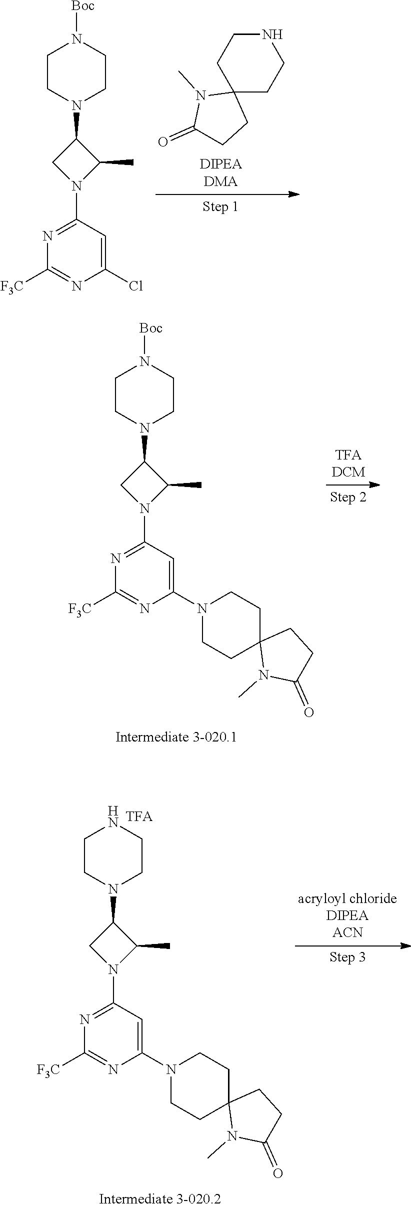

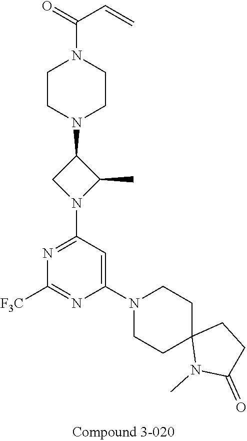 [SMILES: C=CC(=O)N1CCN([C@@H]2CN(c3cc(N4CCC5(CCC(=O)N5C)CC4)nc(C(F)(F)F)n3)[C@@H]2C)CC1]

Step 1: tert-Butyl 4-((2R,3R)-2-methyl-1-(6-(1-methyl-2-oxo-1,8-diazaspiro[4.5]decan-8-yl)-2-(trifluoromethyl)pyrimidin-4-yl)azetidin-3-yl)piperazine-1-carboxylate, Intermediate 3-020.1. To a solution of tert-butyl 4-((2R,3R)-1-(6-chloro-2-(trifluoromethyl)pyrimidin-4-yl)-2-methylazetidin-3-yl)piperazine-1-carboxylate (300 mg, 0.70 mmol), DIPEA (0.360 mL, 2.07 mmol), and DMA (3 mL) was added 1-methyl-1,8-diazaspiro[4.5]decan-2-one hydrochloride (169 mg, 0.83 mmol). The reaction was stirred at 120° C. for 16 h. The crude reaction was purified by reverse-phase HPLC eluting with a gradient of 20-70% ACN (0.1% TFA) in H₂O (0.1% TFA) over 20 min to give Intermediate 3-020.1. MS (ESI) m/z: 568.2 (M+H)⁺.

Step 2: 1-Methyl-8-(6-((2R,3R)-2-methyl-3-(piperazin-1-yl)azetidin-1-yl)-2-(trifluoromethyl)pyrimidin-4-yl)-1,8-diazaspiro[4.5]decan-2-one (TFA salt), Intermediate 3-020.2. To a solution of Intermediate 3-020.1 (302 mg, 0.53 mmol) and DCM (5 mL) was added TFA (0.5 mL). The reaction was stirred at rt for 20 min, then concentrated in vacuo to give Intermediate 3-020.2, as a TFA salt. MS (ESI) m/z: 468.1 (M+H)⁺.

Step 3: 8-(6-((2R,3R)-3-(4-Acryloylpiperazin-1-yl)-2-methylazetidin-1-yl)-2-(trifluoromethyl)pyrimidin-4-yl)-1-methyl-1,8-diazaspiro[4.5]decan-2-one, Compound 3-020. To a solution of Intermediate 3-020.2 (249 mg, 0.53 mmol) and ACN (5 mL) was added DIPEA (0.46 mL, 2.66 mmol) followed by acryloyl chloride (0.026 mL, 0.32 mmol). The reaction was stirred at rt for 20 min. The reaction was purified by reverse-phase preparative HPLC eluting with a gradient of 20-70% ACN (0.1% TFA) in H₂O (0.1% TFA) over 20 min to give Compound 3-020. MS (ESI) m/z: 522.0 (M+H)⁺. ¹H NMR (400 MHz, DMSO-d₆) δ 6.82 (dd, J=16.7, 10.5 Hz, 1H), 6.15 (dd, J=16.6, 2.1 Hz, 1H), 5.69-5.78 (m, 3H), 4.63 (br s, 3H), 4.31-4.46 (m, 4H), 4.07 (br s, 3H), 3.59-3.88 (m, 3H), 2.98 (br t, J=13.6 Hz, 2H), 2.55-2.62 (m, 3H), 2.28 (t, J=7.9 Hz, 2H), 2.00 (t, J=8.0 Hz, 2H), 1.72-1.84 (m, 2H), 1.39-1.59 (m, 5H). ¹⁹F NMR (377 MHz, DMSO-d₆) δ −70.27 (s, 3F), −74.86-−74.19 (m, 3F, from 1 equiv. of TFA).

### Example 3-021: 1-(4-((2R,3R)-1-(6-(2-Oxa-9-azaspiro[5.5]undec-4-en-9-yl)-2-(trifluoromethyl)pyrimidin-4-yl)-2-methylazetidin-3-yl)piperazin-1-yl) prop-2-en-1-one

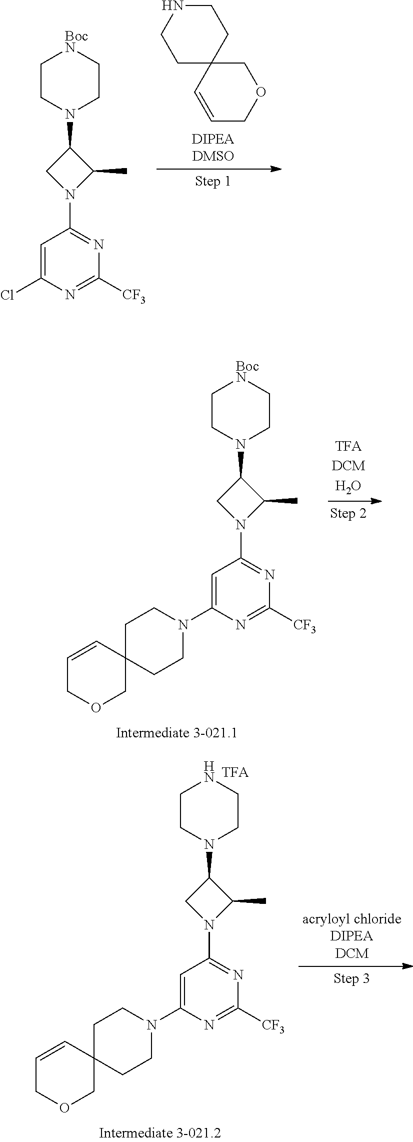 [SMILES: C[C@@H]1[C@H](N2CCN(C(=O)OC(C)(C)C)CC2)CN1c1cc(Cl)nc(C(F)(F)F)n1 ; C1=CC2(CCNCC2)COC1 ; C[C@@H]1[C@H](N2CCN(C(=O)OC(C)(C)C)CC2)CN1c1cc(N2CCC3(C=CCOC3)CC2)nc(C(F)(F)F)n1 ; C[C@@H]1[C@H](N2CCNCC2)CN1c1cc(N2CCC3(C=CCOC3)CC2)nc(C(F)(F)F)n1]

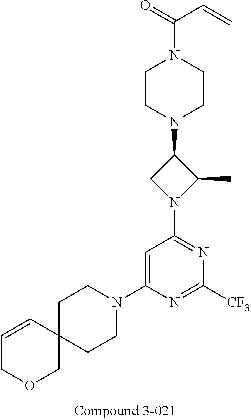 [SMILES: C=CC(=O)N1CCN([C@@H]2CN(c3cc(N4CCC5(C=CCOC5)CC4)nc(C(F)(F)F)n3)[C@@H]2C)CC1]

Step 1: tert-Butyl 4-((2R,3R)-1-(6-(2-oxa-9-azaspiro[5.5]undec-4-en-9-yl)-2-(trifluoromethyl)pyrimidin-4-yl)-2-methylazetidin-3-yl)piperazine-1-carboxylate, Intermediate 3-021.1. tert-Butyl 4-((2R,3R)-1-(6-chloro-2-(trifluoromethyl)pyrimidin-4-yl)-2-methylazetidin-3-yl)piperazine-1-carboxylate (0.44 g. 1.0 mmol), 2-oxa-9-azaspiro[5.5]undec-4-ene hydrochloride (0.20 g, 1.05 mmol), DMSO (1.5 mL), and DIPEA (0.70 mL, 4.01 mmol) were shaken at 60° C. for 16 h. Approx. 50 mg of 2-oxa-9-azaspiro[5.5]undec-4-ene hydrochloride was added. The reaction was shaken at 60° C. for additional 2 h. The crude material was purified by chromatography, eluting with a gradient of 0% to 95% H₂O (0.1% HCOOH) in ACN (0.1% HCOOH)) to provide Intermediate 3-021.1. MS (ESI) m/z: 553.4 (M+H)⁺. ¹H NMR (400 MHz, DMSO-d₆) δ 1.30-1.52 (m, 17H), 2.23 (br s, 4H), 3.18-3.26 (m, 1H), 3.55 (s, 2H), 3.56-3.68 (m, 3H), 3.79-3.88 (m, 1H), 3.89-3.96 (m, 1 H), 4.04 (t, J=2.15 Hz, 2H), 4.47 (t, J=6.62 Hz, 1H), 5.57 (s, 1H), 5.76 (dt, J=10.31, 2.41 Hz, 1H), 5.87 (br d, J=10.37 Hz, 1H).

Step 2: 9-(6-((2R,3R)-2-Methyl-3-(piperazin-1-yl)azetidin-1-yl)-2-(trifluoromethyl)pyrimidin-4-yl)-2-oxa-9-azaspiro[5.5]undec-4-ene, Intermediate 3-021.2. Intermediate 3-021.1 (0.35 g, 0.64 mmol), DCM (10 mL), H₂O (0.2 mL) and TFA (3 mL, 39 mmol) were stirred at rt for 2 h and the solvent was evaporated. The crude material was purified by silica gel chromatography, eluting with a gradient of 95% heptane/5% EtOAc to 100% EtOAc to 90% EtOAc/10% MeOH to afford Intermediate 3-021.2. MS (ESI) m/z 453.4 (M+H)⁺. ¹H NMR (400 MHz, DMSO-d₆) δ 1.35 (d, J=6.32 Hz, 3H), 1.46 (br t, J=5.19 Hz, 4H), 2.21 (br s, 4H), 2.73 (t, J=4.59 Hz, 4H), 3.14-3.21 (m, 1H), 3.54 (s, 2H), 3.55-3.68 (m, 4H), 3.76-3.85 (m, 1H), 3.85-3.95 (m, 1H), 4.03-4.05 (m, 2H), 4.45 (br t, J=6.62 Hz, 1H), 5.56 (s, 1H), 5.75 (dt, J=10.28, 2.49 Hz, 1H), 5.87 (br d, J=10.37 Hz, 1H).

Step 3: 1-(4-((2R,3R)-1-(6-(2-Oxa-9-azaspiro[5.5]undec-4-en-9-yl)-2-(trifluoromethyl)pyrimidin-4-yl)-2-methylazetidin-3-yl)piperazin-1-yl) prop-2-en-1-one, Compound 3-021. To a 50 mL flask under N₂ were added Intermediate 3-021.2 (0.15 g, 0.33 mmol), DCM (5 mL), and DIPEA (250 μL, 1.4 mmol). The mixture was stirred at 0° C. Acryloyl chloride (0.038 g, 0.42 mmol) was dissolved in DCM (2 mL) and added dropwise to the reaction mixture at 0° C. The reaction was stirred for total 15 min at 0° C. and the solvent was removed. The crude material was purified by chromatography, eluting with a gradient of H₂O (0.1% HCOOH) and ACN (0.1% HCOOH) from 95%/0% to 20%/80% to afford Compound 3-021. MS (ESI) m/z: 507.3 (M+H)⁺. ¹H NMR (400 MHz, DMSO-d₆) δ 1.39 (d, J=6.32 Hz, 3H), 1.46 (br t, J=5.36 Hz, 4H), 2.29 (br s, 4H), 3.24 (q, J=7.15 Hz, 1H), 3.49-3.69 (m, 10H), 3.80-3.98 (m, 2H), 4.04 (1, J=2.09 Hz, 2H), 4.48 (t, J=6.62 Hz, 1H), 5.58 (s, 1H), 5.66-5.78 (m, 2H), 5.84-5.90 (m, 1H), 6.11 (dd, J=16.69, 2.38 Hz, 1H), 6.80 (dd, J=16.75, 10.43 Hz, 1H).

Examples in the table below were prepared in a manner similar to that described above.

TABLE 2-2

| | | | MS (ESI) |
|---|---|---|---|
| Compound | | | m/z: |
| No. | Chemical Structure | Name | (M + H)⁺ |
| 3-022 | 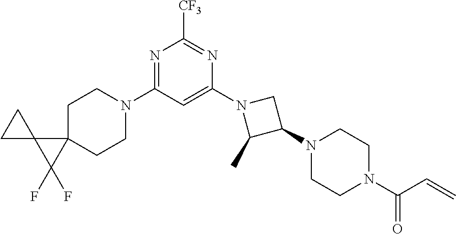 [SMILES: C=CC(=O)N1CCN([C@@H]2CN(c3cc(N4CCC5(CC4)C(F)(F)C54CC4)nc(C(F)(F)F)n3)[C@@H]2C)CC1] | 1-(4-((2R,3R)-1-(6-(10.10- difluoro-7- azadispiro[2.0.5⁴.1³]decan-7- yl)-2- (trifluoromethyl)pyrimidin-4- yl)-2-methylazetidin-3- yl)piperazin-1-yl)prop-2-en-1- one | 527.3 |
| 3-023 | 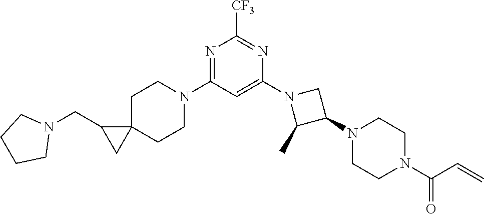 [SMILES: C=CC(=O)N1CCN([C@@H]2CN(c3cc(N4CCC5(CC4)CC5CN4CCCC4)nc(C(F)(F)F)n3)[C@@H]2C)CC1] | 1-(4-((2R,3R)-2-methyl-1-(6- (1-(pyrrolidin-1-ylmethyl)-6- azaspiro[2.5]octan-6-yl)-2- (trifluoromethyl)pyrimidin-4- yl)azetidin-3-yl)piperazin-1- yl)prop-2-en-1-one | 548.3 |
| 3-024 | 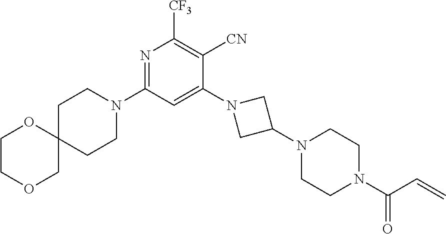 [SMILES: C=CC(=O)N1CCN(C2CN(c3cc(N4CCC5(CC4)COCCO5)nc(C(F)(F)F)c3C#N)C2)CC1] | 4-(3-(4-acryloylpiperazin-1- yl)azetidin-1-yl)-6-(1,4-dioxa- 9-azaspiro[5.5]undecan-9-yl)- 2- (trifluoromethyl)nicotinonitrile | 521.2 |
| 3-025 | 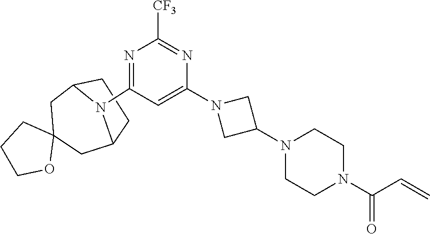 [SMILES: C=CC(=O)N1CCN(C2CN(c3cc(N4C5CCC4CC4(CCCO4)C5)nc(C(F)(F)F)n3)C2)CC1] | 1-(4-(1-(6-(dihydro-3′H-8- azaspiro[bicyclo[3.2.1]octane- 3,2′-furan]-8-yl)-2- (trifluoromethyl)pyrimidin-4- yl)azetidin-3-yl)piperazin-1- yl)prop-2-en-1-one | 507.3 |
| 3-026 | 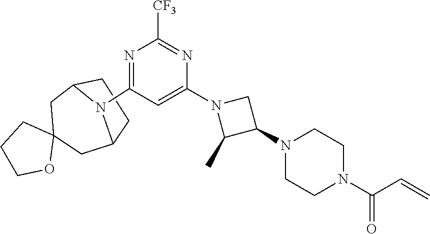 [SMILES: C=CC(=O)N1CCN([C@@H]2CN(c3cc(N4C5CCC4CC4(CCCO4)C5)nc(C(F)(F)F)n3)[C@@H]2C)CC1] | 1-(4-((2R,3R)-1-(6-(dihydro- 3′H-8- azaspiro[bicyclo[3.2.1]octane- 3,2′-furan]-8-yl)-2- (trifluoromethyl)pyrimidin-4- yl)-2-methylazetidin-3- yl)piperazin-1-yl)prop-2-en-1- one | 521.3 |
| 3-027 | 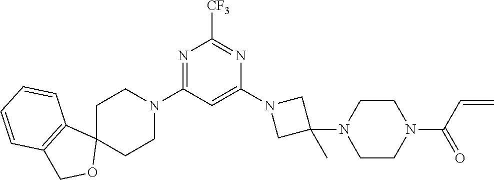 [SMILES: C=CC(=O)N1CCN(C2(C)CN(c3cc(N4CCC5(CC4)OCc4ccccc45)nc(C(F)(F)F)n3)C2)CC1] | 1-(4-(1-(6-(3H- spiro[isobenzofuran-1.4′- piperidin]-1′-yl)-2- (trifluoromethyl)pyrimidin-4- yl)-3-methylazetidin-3- yl)piperazin-1-yl)prop-2-en-1- one | 542.6 |
| 3-028 | 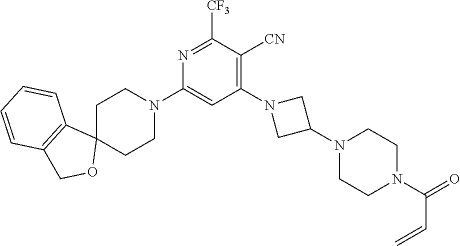 [SMILES: C=CC(=O)N1CCN(C2CN(c3cc(N4CCC5(CC4)OCc4ccccc45)nc(C(F)(F)F)c3C#N)C2)CC1] | 4-(3-(4-acryloylpiperazin-1- yl)azetidin-1-yl)-6-(3H- spiro[isobenzofuran-1,4′- piperidin]-1′-yl)-2- (trifluoromethyl)nicotinonitrile | 553.3 |
| 3-029 | 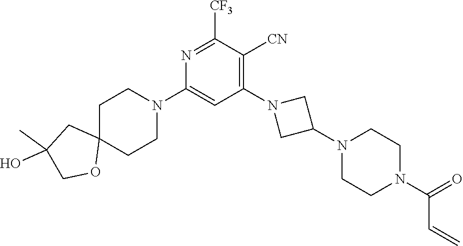 [SMILES: C=CC(=O)N1CCN(C2CN(c3cc(N4CCC5(CC4)CC(C)(O)CO5)nc(C(F)(F)F)c3C#N)C2)CC1] | 4-(3-(4-acryloylpiperazin-1- yl)azetidin-1-yl)-6-(3- hydroxy-3-methyl-1-oxa-8- azaspiro[4.5]decan-8-yl)-2- (trifluoromethyl)nicotinonitrile | 535.3 |
| 3-030 | 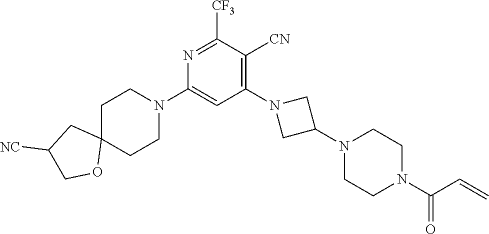 [SMILES: C=CC(=O)N1CCN(C2CN(c3cc(N4CCC5(CC4)CC(C#N)CO5)nc(C(F)(F)F)c3C#N)C2)CC1] | 8-(4-(3-(4-acryloylpiperazin- 1-yl)azetidin-1-yl)-5-cyano-6- (trifluoromethyl)pyridin-2-yl)- 1-oxa-8-azaspiro[4.5]decane- 3-carbonitrile | 530.2 |
| 3-031 | 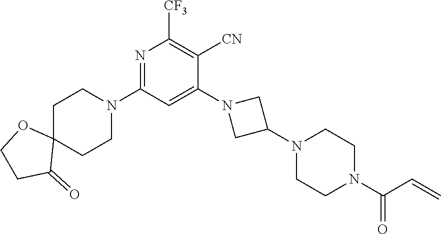 [SMILES: C=CC(=O)N1CCN(C2CN(c3cc(N4CCC5(CC4)OCCC5=O)nc(C(F)(F)F)c3C#N)C2)CC1] | 4-(3-(4-acryloylpiperazin-1- yl)azetidin-1-yl)-6-(4-oxo-1- oxa-8-azaspiro[4.5]decan-8- yl)-2- (trifluoromethyl)nicotinonitrile | 519.2 |
| 3-032 | 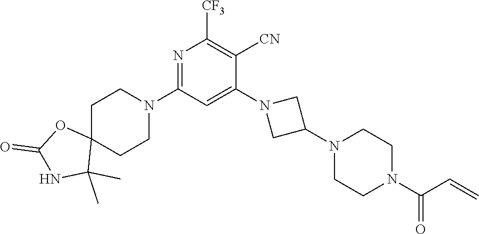 [SMILES: C=CC(=O)N1CCN(C2CN(c3cc(N4CCC5(CC4)OC(=O)NC5(C)C)nc(C(F)(F)F)c3C#N)C2)CC1] | 4-(3-(4-acryloylpiperazin-1- yl)azetidin-1-yl)-6-(4,4- dimethyl-2-oxo-1-oxa-3,8- diazaspiro[4.5]decan-8-yl)-2- (trifluoromethyl)nicotinonitrile | 547.3 |
| 3-033 | 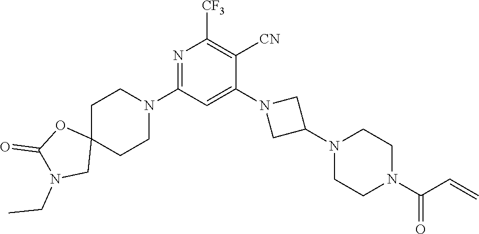 [SMILES: C=CC(=O)N1CCN(C2CN(c3cc(N4CCC5(CC4)CN(CC)C(=O)O5)nc(C(F)(F)F)c3C#N)C2)CC1] | 4-(3-(4-acryloylpiperazin-1- yl)azetidin-1-yl)-6-(3-ethyl-2- oxo-1-oxa-3,8- diazaspiro[4.5]decan-8-yl)-2- (trifluoromethyl)nicotinonitrile | 548.3 |
| 3-034 | 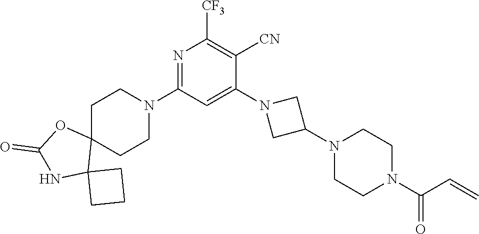 [SMILES: C=CC(=O)N1CCN(C2CN(c3cc(N4CCC5(CC4)OC(=O)NC54CCC4)nc(C(F)(F)F)c3C#N)C2)CC1] | 4-(3-(4-acryloylpiperazin-1- yl)azetidin-1-yl)-6-(12-oxo- 11-oxa-8,13- diazadispiro[3.0.55.34]tridecan- 8-yl)-2- (trifluoromethyl)nicotinonitrile | 560.3 |
| 3-035 | 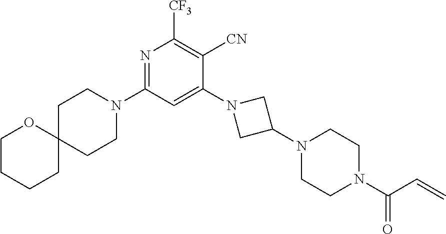 [SMILES: C=CC(=O)N1CCN(C2CN(c3cc(N4CCC5(CCCCO5)CC4)nc(C(F)(F)F)c3C#N)C2)CC1] | 4-(3-(4-acryloylpiperazin-1- yl)azetidin-1-yl)-6-(1-oxa-9- azaspiro[5.5]undecan-9-yl)-2- (trifluoromethyl)nicotinonitrile | 519.3 |
| 3-036 | 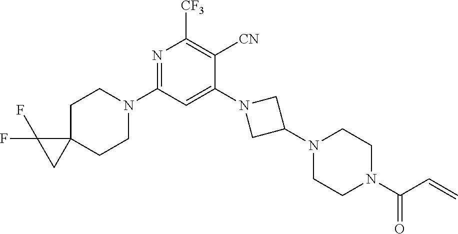 [SMILES: C=CC(=O)N1CCN(C2CN(c3cc(N4CCC5(CC4)CC5(F)F)nc(C(F)(F)F)c3C#N)C2)CC1] | 4-(3-(4-acryloylpiperazin-1- yl)azetidin-1-yl)-6-(1,1- difluoro-6-azaspiro[2.5]octan- 6-yl)-2- (trifluoromethyl)nicotinonitrile | 511.2 |
| 3-037 | 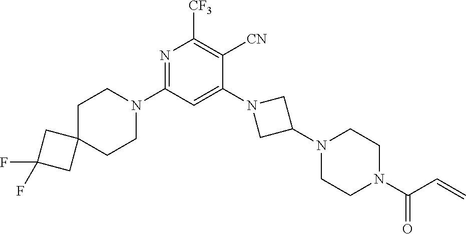 [SMILES: C=CC(=O)N1CCN(C2CN(c3cc(N4CCC5(CC4)CC(F)(F)C5)nc(C(F)(F)F)c3C#N)C2)CC1] | 4-(3-(4-acryloylpiperazin-1- yl)azetidin-1-yl)-6-(2,2- difluoro-7-azaspiro[3.5]nonan- 7-yl)-2- (trifluoromethyl)nicotinonitrile | 525.2 |
| 3-038 | 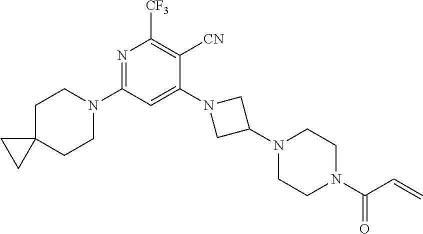 [SMILES: C=CC(=O)N1CCN(C2CN(c3cc(N4CCC5(CC4)CC5)nc(C(F)(F)F)c3C#N)C2)CC1] | 4-(3-(4-acryloylpiperazin-1- yl)azetidin-1-yl)-6-(6- azaspiro[2.5]octan-6-yl)-2- (trifluoromethyl)nicotinonitrile | 475.2 |
| 3-039 | 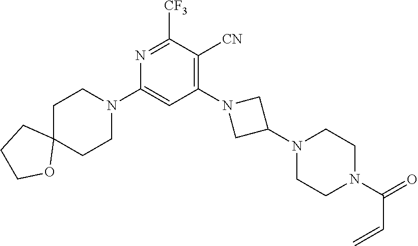 [SMILES: C=CC(=O)N1CCN(C2CN(c3cc(N4CCC5(CCCO5)CC4)nc(C(F)(F)F)c3C#N)C2)CC1] | 4-(3-(4-acryloylpiperazin-1- yl)azetidin-1-yl)-6-(1-oxa-8- azaspiro[4.5]decan-8-yl)-2- (trifluoromethyl)nicotinonitrile | 505.3 |
| 3-040 | 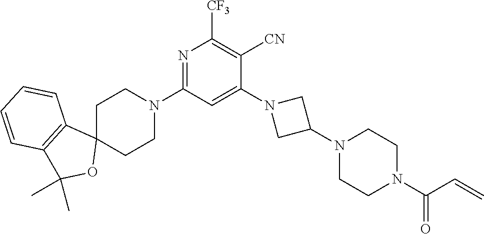 [SMILES: C=CC(=O)N1CCN(C2CN(c3cc(N4CCC5(CC4)OC(C)(C)c4ccccc45)nc(C(F)(F)F)c3C#N)C2)CC1] | 4-(3-(4-acryloylpiperazin-1- yl)azetidin-1-yl)-6-(3,3- dimethyl-3H- spiro[isobenzofuran-1,4′- piperidin]-1′-yl)-2- (trifluoromethyl)nicotinonitrile | 581.3 |
| 3-041 | 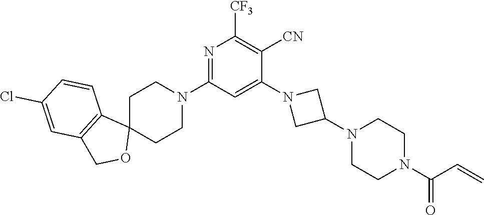 [SMILES: C=CC(=O)N1CCN(C2CN(c3cc(N4CCC5(CC4)OCc4cc(Cl)ccc45)nc(C(F)(F)F)c3C#N)C2)CC1] | 4-(3-(4-acryloylpiperazin-1- yl)azetidin-1-yl)-6-(5-chloro- 3H-spiro[isobenzofuran-1,4′- piperidin]-1′-yl)-2- (trifluoromethyl)nicotinonitrile | 587.2 |
| 3-042 | 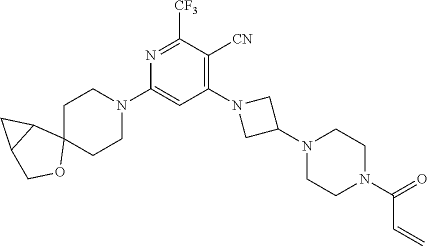 [SMILES: C=CC(=O)N1CCN(C2CN(c3cc(N4CCC5(CC4)OCC4CC45)nc(C(F)(F)F)c3C#N)C2)CC1] | 4-(3-(4-acryloylpiperazin-1- yl)azetidin-1-yl)-6-(3- oxaspiro[bicyclo[3.1.0]hexane- 2,4′-piperidin]-1′-yl)-2- (trifluoromethyl)nicotinonitrile | 517.3 |
| 3-043 | 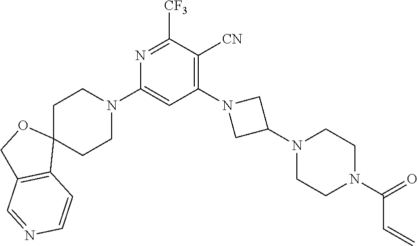 [SMILES: C=CC(=O)N1CCN(C2CN(c3cc(N4CCC5(CC4)OCc4cnccc45)nc(C(F)(F)F)c3C#N)C2)CC1] | 4-(3-(4-acryloylpiperazin-1- vlazetidin-1-yl)-6-(3H- spiro[furo[3,4-c]pyridine-1,4′- piperidin]-1′-yl)-2- (trifluoromethyl)nicotinonitrile | 554.2 |
| 3-044 | 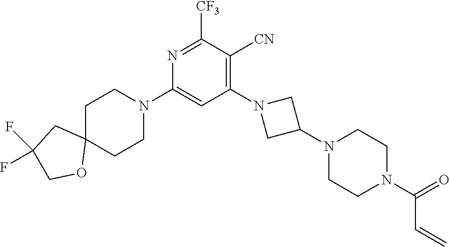 [SMILES: C=CC(=O)N1CCN(C2CN(c3cc(N4CCC5(CC4)CC(F)(F)CO5)nc(C(F)(F)F)c3C#N)C2)CC1] | 4-(3-(4-acryloylpiperazin-1- yl)azetidin-1-yl)-6-(3,3- difluoro-1-oxa-8- azaspiro[4.5]decan-8-yl)-2- (trifluoromethyl)nicotinonitrile | 541.2 |
| 3-045 | 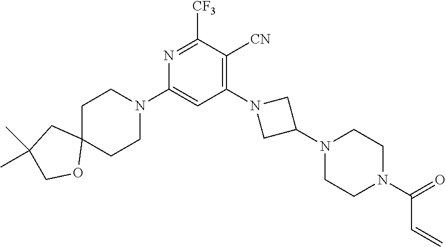 [SMILES: C=CC(=O)N1CCN(C2CN(c3cc(N4CCC5(CC4)CC(C)(C)CO5)nc(C(F)(F)F)c3C#N)C2)CC1] | 4-(3-(4-acryloylpiperazin-1- yl)azetidin-1-yl)-6-(3,3- dimethyl-1-oxa-8- azaspiro[4.5]decan-8-yl)-2- (trifluoromethyl)nicotinonitrile | 533.2 |

### Example 3-046: 4-(3-(4-Acryloylpiperazin-1-yl)azetidin-1-yl)-6-(4H-spiro[furo[3,4-d]thiazole-6,4′-piperidin]-1′-yl)-2-(trifluoromethyl)nicotinonitrile

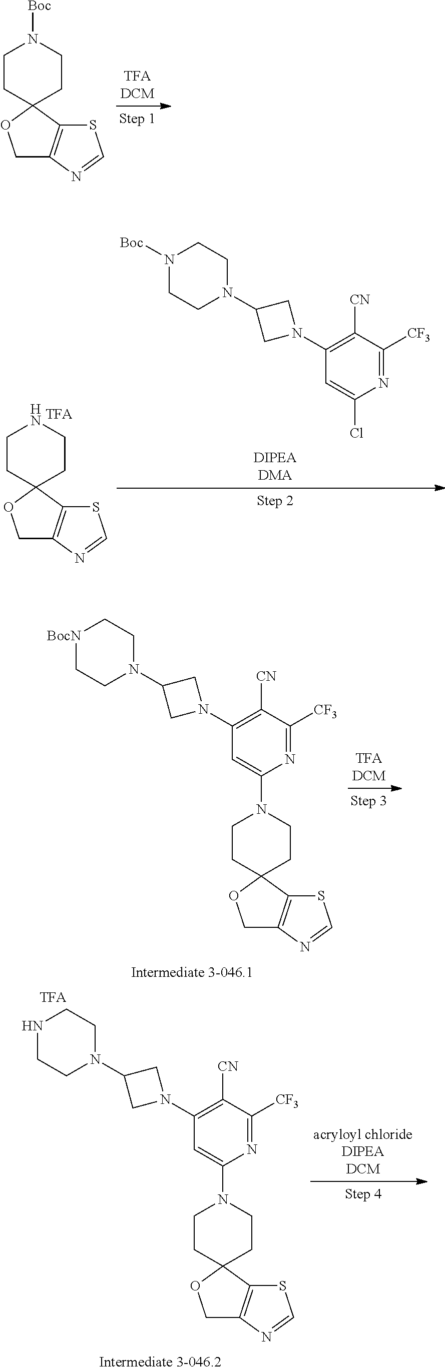 [SMILES: CC(C)(C)OC(=O)N1CCC2(CC1)OCc1ncsc12 ; c1nc2c(s1)C1(CCNCC1)OC2 ; CC(C)(C)OC(=O)N1CCN(C2CN(c3cc(Cl)nc(C(F)(F)F)c3C#N)C2)CC1 ; CC(C)(C)OC(=O)N1CCN(C2CN(c3cc(N4CCC5(CC4)OCc4ncsc45)nc(C(F)(F)F)c3C#N)C2)CC1 ; N#Cc1c(N2CC(N3CCNCC3)C2)cc(N2CCC3(CC2)OCc2ncsc23)nc1C(F)(F)F]

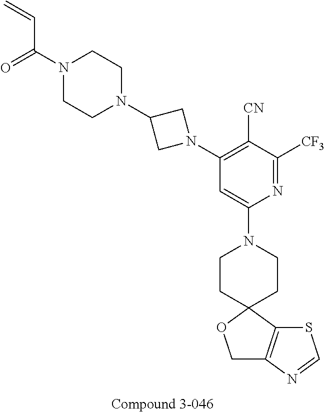 [SMILES: C=CC(=O)N1CCN(C2CN(c3cc(N4CCC5(CC4)OCc4ncsc45)nc(C(F)(F)F)c3C#N)C2)CC1]

Step 1: tert-Butyl 4H-spiro[furo[3,4-d]thiazole-6,4′-piperidine]-1′-carboxylate (TFA salt), Intermediate 3-046.1. tert-Butyl 4H-spiro[furo[3,4-d]thiazole-6,4′-piperidine]-1′-carboxylate (68 mg, 0.23 mmol), DCM (1.2 mL), and TFA (342 μL. 4.59 mmol) were stirred at rt for 30 min, concentrated, and used in the next step.

Step 2: ter-Butyl 4-(1-(3-cyano-6-(4H-spiro[furo[3,4-d]thiazole-6,4′-piperidin]-1′-yl)-2-(trifluoromethyl)pyridin-4-yl)azetidin-3-yl)piperazine-1-carboxylate, Intermediate 3-046.2. To a solution of the crude material from Step 1 in DMA (1.2 mL) at rt was added DIPEA (401 μL, 2.29 mmol). The reaction mixture was stirred for 10 min at rt, and tert-butyl 4-(1-(6-chloro-3-cyano-2-(trifluoromethyl)pyridin-4-yl)azetidin-3-yl)piperazine-1-carboxylate (102 mg, 0.23 mmol) was added. The reaction mixture was stirred at 86° C. overnight. The reaction was quenched by sat. aq. NH₄Cl solution (2 mL) and extracted with EtOAc (3 mL). The organic phase was washed by H₂O (2 mL) and sat. aq. NH₄Cl solution, and the combined aqueous phase was extracted with EtOAc (2 mL). The organic phase was combined and washed with brine (3 mL), dried over Na₂SO₄, and concentrated. The crude material was purified by silica gel chromatography, eluting with a gradient of 1% to 16% acetone in DCM to provide Intermediate 3-046.2. MS (ESI) m/z: 628.0 (M+23)⁺. ¹H NMR (400 MHz, CDCl₃) δ 8.77 (s, 1H), 5.44 (s, 1H), 5.11 (s, 2H), 4.38 (br s, 2H), 4.13 (br s, 2H), 3.93 (br d, J=13.6 Hz, 2H), 3.69-3.80 (m, 2H), 3.49 (br s, 4H), 3.31 (br s, 1H), 2.38 (br s, 4H), 1.89-2.06 (m, 4H), 1.49 (s, 9H).

Step 3: 4-(3-(Piperazin-1-yl)azetidin-1-yl)-6-(4H-spiro[furo[3,4-d]thiazole-6,4′-piperidin]-1′-yl)-2-(trifluoromethyl)nicotinonitrile, Intermediate 3-046.3. Intermediate 3-046.2 (11 mg, 0.018 mmol), DCM (0.2 mL) and TFA (27 μL, 0.36 mmol) were stirred at rt for 1 h, and concentrated to provide crude product containing Intermediate 3-046.3, which was used in the next step.

Step 4: 4-(3-(4-Acryloylpiperazin-1-yl)azetidin-1-yl)-6-(4H-spiro[furo[3,4-d]thiazole-6,4′-piperidin]-1′-yl)-2-(trifluoromethyl)nicotinonitrile, Compound 3-046. To the crude mixture of Step 3 was added 0.2 mL DCM followed by DIPEA (32 μL, 0.18 mmol) and acryloyl chloride (0.2 M in DCM, 109 μL, 0.022 mmol) at rt, and stirred for 15 min. The reaction solution was quenched with sat. aq. NH₄Cl solution and brine and extracted with DCM. The organic phase was dried over Na₂SO₄ and concentrated. The crude material was purified by silica gel chromatography, eluting with a gradient of 0% to 6% MeOH in DCM to provide Compound 3-046. MS (ESI) m/z: 560.2 (M+H)⁺. ¹H NMR (400 MHz, CDCl₃) δ 8.78 (s, 1H), 6.58 (dd, J=16.8, 10.6 Hz, 1H), 6.32 (dd, J=16.9, 1.8 Hz, 1H), 5.74 (dd, J=10.5, 1.8 Hz, 1H), 5.45 (s, 1H), 5.11 (s, 2H), 4.40 (br s, 2H), 4.14 (br s, 2H), 3.94 (dt, J=13.5, 4.7 Hz, 2H), 3.74 (ddd, J=13.5, 9.3, 3.7 Hz, 4H), 3.64 (br s, 2H), 3.33 (br s, 1H), 2.44 (br s, 4H), 1.90-2.06 (m, 4H). ¹⁹F NMR (377 MHz, CDCl₃) δ −66.72 (s, 3F).

Examples in the table below were prepared in a manner similar to that described above.

TABLE 2-3

| Compound | | | MS (ESI) |
|---|---|---|---|
| No. | Chemical Structure | Name | m/z: (M + H)⁺ |
| 3-047 | 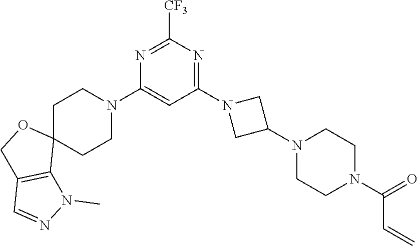 [SMILES: C=CC(=O)N1CCN(C2CN(c3cc(N4CCC5(CC4)OCc4cnn(C)c45)nc(C(F)(F)F)n3)C2)CC1] | 1-(4-(1-(6-(1-methyl-1,4- dihydrospiro[furo[3,4- c]pyrazole-6,4′-piperidin]-1′- yl)-2- (trifluoromethyl)pyrimidin-4- yl)azetidin-3-yl)piperazin-1- yl)prop-2-en-1-one | 533.0 |
| 3-048 | 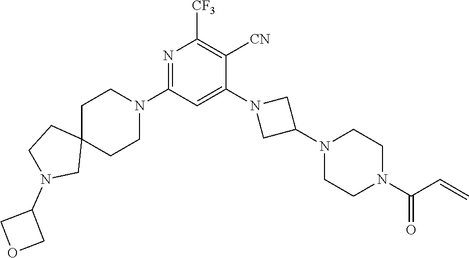 [SMILES: C=CC(=O)N1CCN(C2CN(c3cc(N4CCC5(CC4)CCN(C4COC4)C5)nc(C(F)(F)F)c3C#N)C2)CC1] | 4-(3-(4-acryloylpiperazin-1- yl)azetidin-1-yl)-6-(2- (oxetan-3-yl)-2,8- diazaspiro[4.5]decan-8-yl)-2- (trifluoromethyl)nicotinonitrile | 560.2 |
| 3-049 | 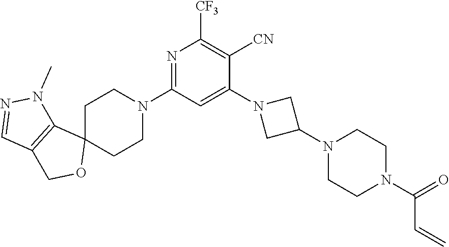 [SMILES: C=CC(=O)N1CCN(C2CN(c3cc(N4CCC5(CC4)OCc4cnn(C)c45)nc(C(F)(F)F)c3C#N)C2)CC1] | 4-(3-(4-acryloylpiperazin-1- yl)azetidin-1-yl)-6-(1-methyl- 1,4-dihydrospiro[furo[3,4- c]pyrazole-6,4′-piperidin]-1′- yl)-2- (trifluoromethyl)nicotinonitrile | 557.2 |

### Example 3-050: 1-(4-((2R,3R)-1-(2-(Difluoromethyl)-6-(4-methyl1-oxa-8-azaspiro[4.5]dec3-en-8-yl)pyrimidin-4-yl)-2-methylazetidin-3-yl)piperazin-1-yl)prop2-en-1-one

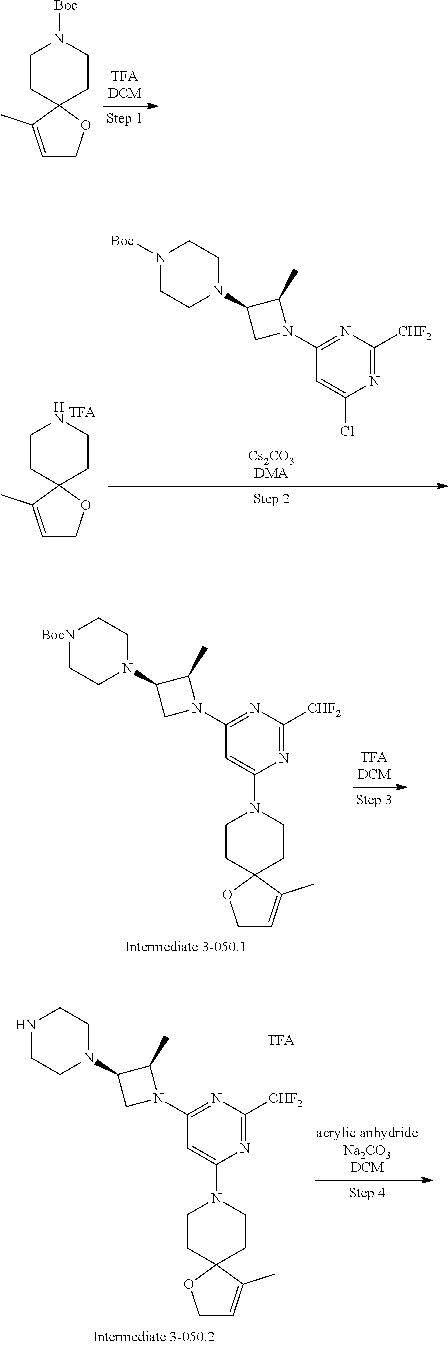 [SMILES: CC1=CCOC12CCN(C(=O)OC(C)(C)C)CC2 ; CC1=CCOC12CCNCC2 ; C[C@@H]1[C@H](N2CCN(C(=O)OC(C)(C)C)CC2)CN1c1cc(Cl)nc(C(F)F)n1 ; CC1=CCOC12CCN(c1cc(N3C[C@@H](N4CCN(C(=O)OC(C)(C)C)CC4)[C@H]3C)nc(C(F)F)n1)CC2 ; CC1=CCOC12CCN(c1cc(N3C[C@@H](N4CCNCC4)[C@H]3C)nc(C(F)F)n1)CC2]

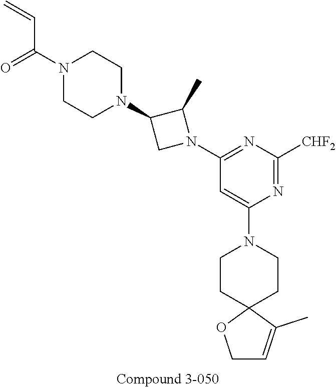 [SMILES: C=CC(=O)N1CCN([C@@H]2CN(c3cc(N4CCC5(CC4)OCC=C5C)nc(C(F)F)n3)[C@@H]2C)CC1]

Step 1: 4-Methyl-1-oxa-8-azaspiro[4.5]dec-3-ene, Intermediate 3-050.1. tert-Butyl 4-methyl-1-oxa-8-azaspiro[4.5]dec-3-ene-8-carboxylate (150 mg, 0.59 mmol). DCM (3 mL), and TFA (883 μL, 11.84 mmol) were stirred at rt for 30 min, concentrated, and used in the following step as is.

Step 2: tert-Butyl 4-((2R,3R)-1-(2-(difluoromethyl)-6-(4-methyl-1-oxa-8-azaspiro[4.5]dec-3-en-8-yl)pyrimidin-4-yl)-2-methylazetidin-3-yl)piperazine-1-carboxylate, Intermediate 3-050.1. To the crude mixture of Step 1 was added Cs₂CO₃ (1.93 g, 5.92 mmol) in DMA (3 mL) at rt. The reaction mixture was stirred for 10 min at rt, and tert-butyl 4-((2R,3R)-1-(2-(difluoromethyl)-6-fluoropyrimidin-4-yl)-2-methylazetidin-3-yl)piperazine-1-carboxylate (214 mg, 0.533 mmol) was added. The reaction mixture was then stirred 16 h at 95° C. The reaction was quenched with sat. aq. NH₄Cl solution (5 mL) and EtOAc (10 mL). The organic phase was washed with H₂O and sat. aq. NH₄Cl solution, and the combined aqueous phase was extracted with EtOAc (3 mL). The combined organic phases were washed with brine (3 mL), dried over Na₂SO₄, and concentrated to provide crude material containing Intermediate3-050.1. The crude material was used in the next step.

Step 3: 8-(2-(Difluoromethyl)-6-((2R,3R)-2-methyl-3-(piperazin-1-yl)azetidin-1-yl)pyrimidin-4-yl)-4-methyl-1-oxa-8-azaspiro[4.5]dec-3-ene, Intermediate 3-050.2. To the crude mixture of Step 2 was added tert-butyl 4-((2R,3R)-1-(2-(difluoromethyl)-6-(4-methyl-1-oxa-8-azaspiro[4.5]dec-3-en-8-yl)pyrimidin-4-yl)-2-methylazetidin-3-yl)piperazine-1-carboxylate (317 mg, 0.59 mmol) and TFA (882 μL, 11.8 mmol) in DCM (5.92 mL). The reaction mixture was stirred at rt for 1 h to provide Intermediate 3-050.2 as a TFA salt, which was taken directly into the next step.

Step 4: 1-(4-((2R,3R)-1-(2-(Difluoromethyl)-6-(4-methyl-1-oxa-8-azaspiro[4.5]dec3-en-8-yl)pyrimidin-4-yl)-2-methylazetidin-3-yl)piperazin-1-yl)prop2-en-1-one, Compound 3-050. To the crude material of Step 3 was added Na₂CO₃ (10% in H₂O, 1.25 mL, 11.84 mmol) followed by acrylic anhydride (2.96 mL, 0.59 mmol) at 0° C. The resulting mixture was stirred for 40 min. The reaction mixture was extracted with DCM (3 mL×2). The organic phase was dried through Na₂SO₄, and concentrated. The residue was purified by prep HPLC eluting with (0.1% HCOOH) H₂O in ACN to provide Compound 3-050. MS (ESI) m/z: 489.0 (M+H)⁺. ¹H NMR (600 MHz, DMSO-d₆) δ 6.80 (dd, J=16.7, 10.5 Hz, 1H), 6.40 (t, J=54.9 Hz, 1H), 6.10 (dd, J=16.7, 2.4 Hz, 1H), 5.68 (dd, J=10.5, 2.3 Hz, 1H), 5.48-5.59 (m, 2H), 4.45 (br d, J=1.8 Hz, 3H), 4.31 (br s, 2H), 3.91 (br t, J=7.8 Hz, 1H), 3.79-3.87 (m, 1H), 3.58 (br d, J=5.6 Hz, 2H), 3.29-3.39 (m, 2H), 3.22 (q, J=7.1 Hz, 1H), 3.02-3.13 (m, 2H), 2.36-2.40 (m, 1H), 2.28 (br s, 3H), 1.66 (td, J=13.4, 4.1 Hz, 2H), 1.62 (d, J=1.6 Hz, 3H), 1.34-1.44 (m, 5H).

### Example 3-051: 4-(3-(4-Acryloylpiperazin-1-yl)azetidin-1-yl)-6-(4-methyl-1-oxa-8-azaspiro[4.5]dec-3-en-8-yl)-2-(trifluoromethyl)nicotinonitrile

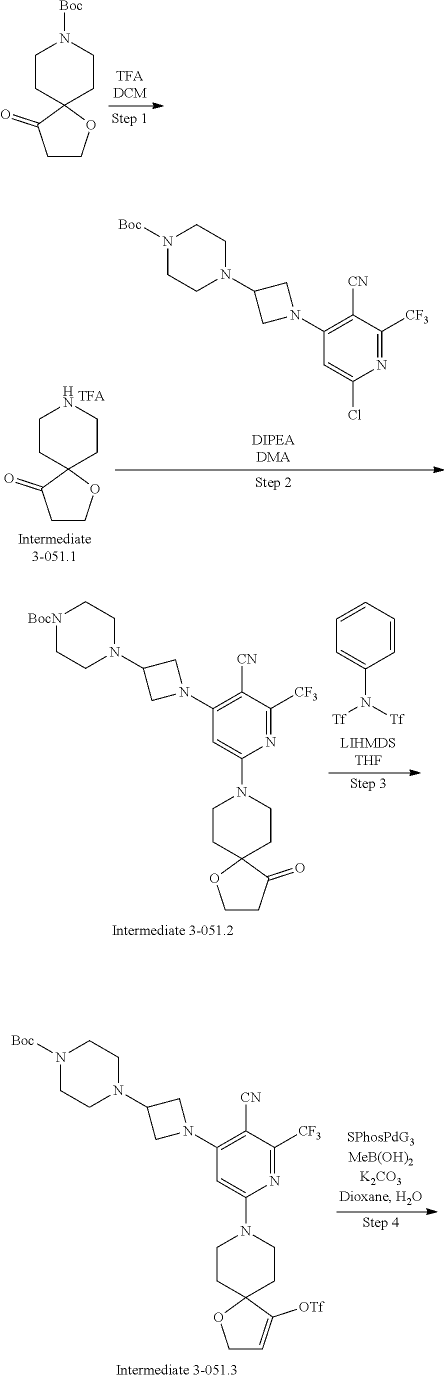

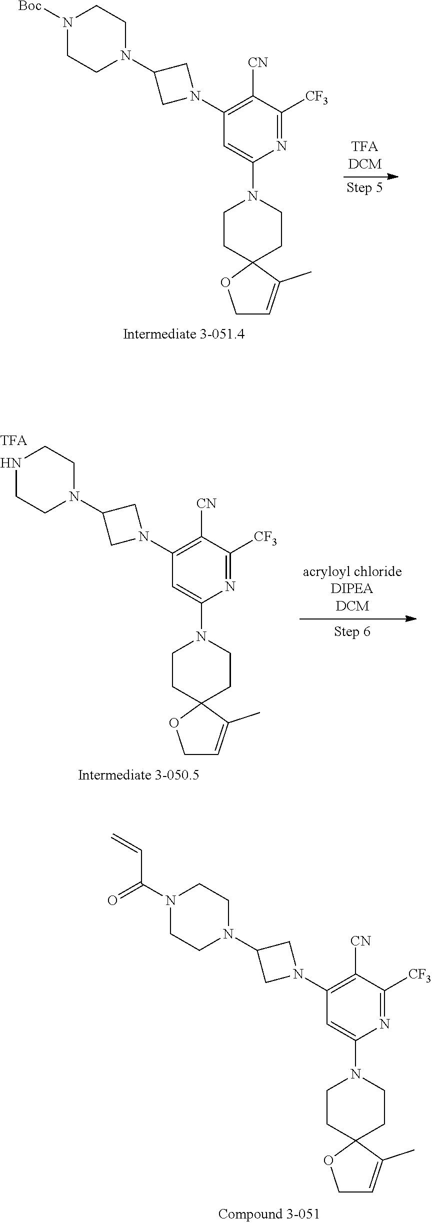 [SMILES: CC1=CCOC12CCN(c1cc(N3CC(N4CCN(C(=O)OC(C)(C)C)CC4)C3)c(C#N)c(C(F)(F)F)n1)CC2 ; CC1=CCOC12CCN(c1cc(N3CC(N4CCNCC4)C3)c(C#N)c(C(F)(F)F)n1)CC2 ; C=CC(=O)N1CCN(C2CN(c3cc(N4CCC5(CC4)OCC=C5C)nc(C(F)(F)F)c3C#N)C2)CC1]

Step 1: 1-Oxa-8-azaspiro[4.5]decan-4-one, Intermediate 3-051.1. tert-Butyl 4-oxo-1-oxa-8-azaspiro[4.5]decane-8-carboxylate (477 mg, 1.87 mmol), DCM (8 mL), and TFA (2.4 mL, 32 mmol) were stirred at rt for 2 h, and concentrated to provide Intermediate 3-051.1 as a TFA salt which was used in the next step.

Step 2: tert-Butyl 4-(1-(3-cyano-6-(4-oxo-1-oxa-8-azaspiro[4.5]decan-8-yl)-2-(trifluoromethyl)pyridin-4-yl)azetidin-3-yl)piperazine-1-carboxylate, Intermediate 3-051.2. To the crude material from Step 1 was added DIPEA (2.8 mL, 16.1 mmol) in DMA (8 mL) at rt. The reaction mixture was stirred for 10 min at rt, and tert-butyl 4-(1-(6-chloro-3-cyano-2-(trifluoromethyl)pyridin-4-yl)azetidin-3-yl)piperazine-1-carboxylate (430 mg, 0.96 mmol) was added. The reaction mixture was then heated at 86° C. for 42 h. The reaction was quenched by sat. aq. NH₄Cl solution (8 mL) and EtOAc (16 mL). The organic phase was washed with H₂O (8 mL) and sat. aq. NH₄Cl solution (2×8 mL), and the combined aqueous extracts were extracted with EtOAc (8 mL). The combined organic extracts were washed with brine (8 mL), dried over Na₂SO₄, and concentrated. The crude material was purified by silica gel chromatography eluting with a gradient of 0% to 5% MeOH in DCM to provide Intermediate 3-051.2. MS (ESI) m/z: 565.0 (M+H)⁺.

Step 3: tert-Butyl 4-(1-(3-cyano-2-(trifluoromethyl)-6-(4-(((trifluoromethyl)sulfonyl)oxy)-1-oxa-8-azaspiro[4,5]dec-3-en-8-yl)pyridin-4-yl)azetidin-3-yl)piperazine-1-carboxylate, Intermediate 3-051.3. N-phenyl bis-trifluoromethane sulfonimide (152 mg, 0.425 mmol) and Intermediate 3-051.2 (200 mg, 0.35 mmol) were combined, the reaction vessel was evacuated and backfilled with Ne, and THF (3 mL) was added. The reaction mixture was cooled to 0° C. and LiHMDS (1.0 M in THF, 0.43 mL, 0.43 mmol) was added. The reaction mixture was stirred at rt for 2 h. Additional equivalents of N-phenyl bis-trifluoromethane sulfonimide and LiHMDS were added (1 equiv each). The reaction mixture was quenched by the addition of sat. aq. NH₄Cl and extracted with EtOAc. The organic extracts were filtered over Na₂SO₄ and concentrated in vacuo. The crude material was purified by chromatography, eluting with a gradient of 0% to 60% EtOAc in heptane, to provide Intermediate 3-051.3. MS (ESI) m/z: 697.2 (M+H)⁺.

Step 4: tert-Butyl 4-(1-(3-cyano-6-(4-methyl-1-oxa-8-azaspiro[4.5]dec-3-en-8-yl)-2-(trifluoromethyl)pyridin-4-yl)azetidin-3-yl)piperazine-1-carboxylate, Intermediate 3-051.4. SPhos Pd G3 (11 mg, 0.012 mmol), K₂CO₃ (34 mg, 0.25 mmol), methylboronic acid (15 mg, 0.25 mmol), and Intermediate 3-051.3 (85 mg, 0.12 mmol) were combined, and the reaction vessel was evacuated and backfilled with N₂. To the reaction mixture was added 1,4-dioxane (2 mL) and H₂O (0.2 mL) and the reaction mixture was stirred at 100° C. for 30 min. The reaction mixture was filtered through Na₂SO₄ and concentrated in vacuo. The crude material was purified by chromatography, eluting with a gradient of 0-60% EtOAc in heptane, to provide Intermediate 3-051.4. MS (ESI) m/s: 563.2 (M+H)⁺.

Step 5: 6-(4-Methyl-1-oxa-8-azaspiro[4.5]dec-3-en-8-yl)-4-(3-(piperazin-1-yl)azetidin-1-yl)-2-(trifluoromethyl)nicotinonitrile (TFA salt), Intermediate 3-051.5. To a solution of Intermediate 3-051.4 (48 mg, 0.086 mmol) in DCM (3 mL) was added TFA (0.5 mL, 0.086 mmol) and the reaction mixture was stirred for 30 min at rt. The reaction mixture was concentrated to provide Intermediate 3-051.5, as a TFA salt. MS (ESI) m/z: 463.0 (M+H)⁻.

Step 6: 4-(3-(4-Acryloylpiperazin-1-yl)azetidin-1-yl)-6-(4-methyl-1-oxa-8-azaspiro[4.5]dec-3-en-8-yl)-2-(trifluoromethyl)nicotinonitrile, Compound 3-051. Intermediate 3-051.5 (TFA salt) was dissolved in DCM (3 mL). To the reaction mixture was added DIPEA (0.15 mL, 0.86 mmol) and acryloyl chloride (0.2 M in DCM, 0.43 mL, 0.086 mmol) and the reaction mixture was stirred at rt for 30 min. The reaction mixture was quenched by the addition of sat. aq. NaHCO₃ and extracted with DCM. The organic extracts were washed with sat. aq. NH₄Cl, dried over Na₂SO₄, and concentrated in vacuo. The crude material was purified by silica gel chromatography, eluting with a gradient of 0-60% EtOAc/EtOH (3:1) in heptane. The material was further purified by reverse-phase preparative HPLC using 0.1% TFA in ACN/H₂O as eluent, with a gradient 10-90% over 15 min. The fractions containing product were free based by washing with sat. aq. Na₂CO₃ and extracted with EtOAc. The organic extracts were washed with brine, dried over Na₂SO₄, filtered, and concentrated in vacuo to provide Compound 3-051. MS (ESI) m/z: 517.2 (M+H)⁺. ¹H NMR (400 MHz, CDCl₃) δ 6.46-6.64 (m, 1H), 6.19-6.38 (m, 1H), 5.62-5.79 (m, 1H), 5.43-5.56 (m, 1H), 5.39 (s, 1H), 4.51-4.59 (m, 2H), 4.24-4.41 (m, 4H), 4.03-4.14 (m, 2H), 3.54-3.79 (m, 4H), 3.15-3.39 (m, 3H), 2.28-2.49 (m, 4H), 1.69-1.80 (m, 2H), 1.64-1.68 (m, 3H), 1.51-1.58 (m, 2H). ¹⁹F NMR (377 MHz, CDCl₃) δ −66.73 (s, 3F).

### Example 3-052: 4-((2R,3R)-3-(4-Acryloylpiperazin-1-yl)-2-methylazetidin-1-yl)-6-(4-methyl-1-oxa-8-azaspiro[4.5]dec-3-en-8-yl)-2-(trifluoromethyl)nicotinonitrile

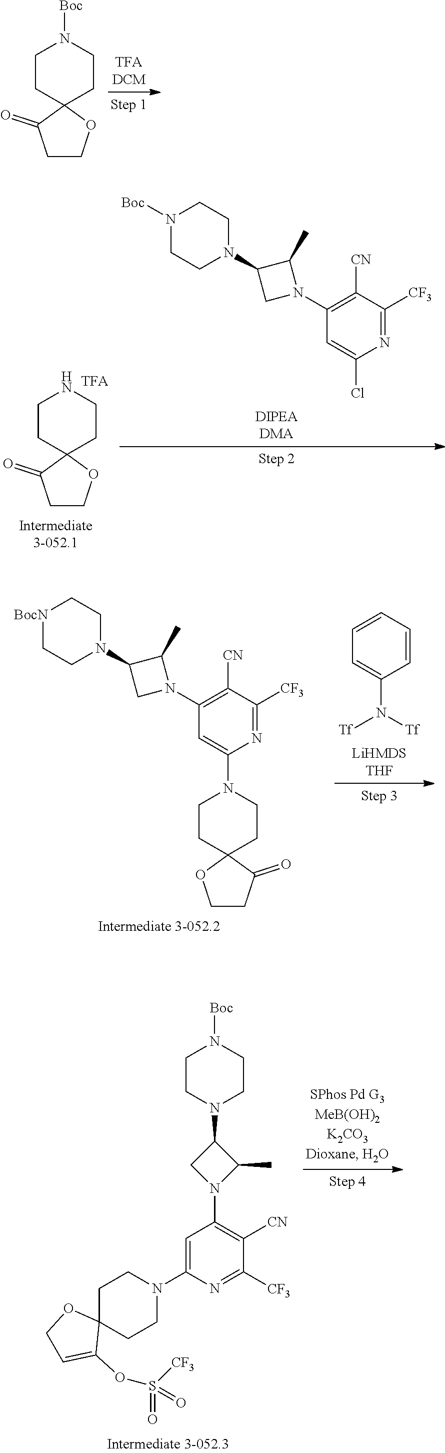

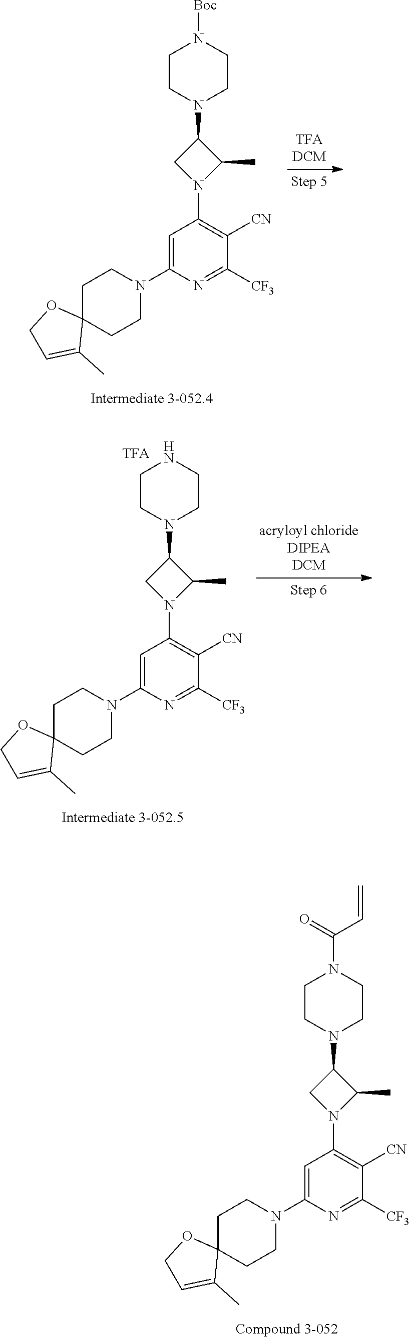 [SMILES: CC1=CCOC12CCN(c1cc(N3C[C@@H](N4CCN(C(=O)OC(C)(C)C)CC4)[C@H]3C)c(C#N)c(C(F)(F)F)n1)CC2 ; CC1=CCOC12CCN(c1cc(N3C[C@@H](N4CCNCC4)[C@H]3C)c(C#N)c(C(F)(F)F)n1)CC2 ; C=CC(=O)N1CCN([C@@H]2CN(c3cc(N4CCC5(CC4)OCC=C5C)nc(C(F)(F)F)c3C#N)[C@@H]2C)CC1]

Step 1: 1-Oxa-8-azaspiro[4.5]decan-4-one (TFA salt), Intermediate 3-052.1. tert-Butyl 4-oxo-1-oxa-8-azaspiro[4.5]decane-8-carboxylate (563 mg, 2.21 mmol), DCM (11 mL), and TFA (3.29 mL, 44.1 mmol) were stirred at rt for 30 min. The mixture was concentrated to provide Intermediate 3-052.1 and the crude material used in the next step. MS (ESI) m/z: 156.2 (M+H)⁺.

Step 2: tert-Butyl 4-((2R,3R)-1-(3-cyano-6-(4-oxo-1-oxa-8-azaspiro[4.5]decan-8-yl)-2-(trifluoromethyl)pyridin-4-yl)-2-methylazetidin-3-yl)piperazine-1-carboxylate, Intermediate 3-052.2. To the crude material from step 1 was added DIPEA (3.85 mL, 22.05 mmol) in DMA (11 mL) at rt. The reaction mixture was stirred for 10 min at rt, and tert-butyl 4-((2R,3R)-1-(6-chloro-3-cyano-2-(trifluoromethyl)pyridin-4-yl)-2-methylazetidin-3-yl)piperazine-1-carboxylate (811 mg, 1.78 mmol) was added. The reaction mixture was then heated at 95° C. for 42 h. The reaction was quenched by adding sat. aq. NH₄Cl solution (20 mL) and EtOAc (40 mL). The organic phase was washed with H₂O and sat. aq. NH₄Cl solution, and the combined aqueous phases were extracted with EtOAc (20 mL). The organic phases were combined and washed with brine (15 mL), dried over Na₂SO₄, and concentrated. The crude material was purified by silica gel chromatography, eluting with a gradient of 10-75% EtOAc in heptane to provide Intermediate 3-052.2. MS (ESI) m/z: 579.0 (M+H)⁺.

Step 3: tert-Butyl 4-((2R,3R)-1-(3-cyano-2-(trifluoromethyl)-6-(4-(((trifluoromethyl)sulfonyl)oxy)-1-oxa-8-azaspiro[4.5]dec-3-en-8-yl)pyridin-4-yl)-2-methylazetidin-3-yl)piperazine 1-carboxylate, Intermediate 3-052.3. N-phenyl bis-trifluoromethane sulfonimide (0.678 g, 1.90 mmol) and Intermediate 3-052.2 (0.914 g, 1.58 mmol) were combined, and the reaction vessel was evacuated and backfilled with Ne. To the reaction mixture was added THF (4 mL) and cooled to 0° C. To the reaction mixture was added LiHMDS (1.0 M in THF, 1.90 mL, 1.90 mmol) and the reaction mixture was stirred at it for 1 h. The reaction mixture was quenched by the addition of sat. aq. NH₄Cl and extracted with EtOAc. The organic extracts were washed with brine, dried over Na₂SO₄, filtered, and concentrated in vacuo. The crude material was purified by chromatography, eluting with a gradient of 0-40% EtOAc in heptane, to provide Intermediate 3-052.3. MS (ESI) m/z: 711.2 (M+H)⁺.

Step 4: tert-Butyl 4-((2R,3R)-1-(3-cyano-6-(4-methyl-1-oxa-8-azaspiro[4.5]dec-3-on-8-yl)-2-(trifluoromethyl)pyridin-4-yl)-2-methylazetidin-3-yl)piperazine-1-carboxylate (Intermediate 3-052.4). SPhos Pd G3 (18.3 mg, 0.021 mmol), K₂CO₃ (58 mg, 0.42 mmol), methylboronic acid (50.5 mg, 0.84 mmol), and Intermediate 3-052.3 (150 mg, 0.21 mmol) were combined and the reaction vessel was evacuated and backfilled with N₂. To the reaction mixture was added 1,4-dioxane (4 mL) and H₂O (0.4 mL) and the reaction mixture was stirred at 100° C. for 1 h. The reaction mixture was filtered through Na₂SO₄ and concentrated in vacuo. The crude material was purified by chromatography, eluting with a gradient of 0-40% EtOAc in heptane, to provide Intermediate 3-052.4. MS (ESI) m/z: 577.2 (M+H)⁺.

Step 5: 4-((2R,3R)-2-Methyl-3-(piperazin-1-yl)azetidin-1-yl)-6-(4-methyl-1-oxa-8-azaspiro[4.5]dec-3-en-8-yl)-2-(trifluoromethyl)nicotinonitrile, Intermediate 3-052.5 (TFA salt). To a solution of Intermediate 3-052.4 (79 mg, 0.14 mmol) in DCM (3 mL) was added TFA (0.5 mL, 0.14 mmol) and the reaction mixture was stirred for 30 min at rt. The reaction was concentrated to provide Intermediate 3-052.5 (TFA salt). MS (ESI) m/z: 477.4 (M+H)⁺.

Step 6: 4-((2R,3R)-3-(4-Acryloylpiperazin-1-yl)-2-methylazetidin-1-yl)-6-(4-methyl-1-oxa-8-azaspiro[4.5]dec-3-en-8-yl)-2-(trifluoromethyl)nicotinonitrile, Compound 3-052. Intermediate 3-052.5 (TFA salt) was redissolved in DCM (3 mL). To the reaction mixture was added DIPEA (0.24 mL, 1.37 mmol) and acryloyl chloride (0.2 M in DCM, 0.68 mL, 0.14 mmol) and the reaction mixture was stirred at rt for 30 min. The reaction mixture was quenched by the addition of sat. aq. NaHCO₃ and extracted with DCM. The organic extracts were washed with sat. aq. NH₄Cl, dried over Na₂SO₄, and concentrated in vacuo. The crude material was purified by reverse-phase preparative HPLC eluting with a gradient 10% to 90% of (0.1% TFA) ACN in H₂O, over 15 min. The fractions containing product were free based by washing with sat. aq. Na₂CO₃ and extracted with EtOAc. The organic extracts were washed with brine, dried over Na₂SO₄, filtered, and concentrated in vacuo to provide Compound 3-052. MS (ESI) m/z: 531.0 (M+H)⁺. ¹H NMR (400 MHz, CDCl₃) δ 6.46-6.72 (m, 1H), 6.16-6.42 (m, 1H), 5.64-5.83 (m, 1H), 5.50 (d, J=1.5 Hz, 1H), 5.40-5.45 (m, 1H), 4.83-4.93 (m, 1H), 4.50-4.59 (m, 2H), 4.22-4.38 (m, 2H), 4.05 (br d, J=6.8 Hz, 2H), 3.52-3.84 (m. 4H), 3.19-3.41 (m, 3H), 2.35 (br d, J=3.1 Hz, 4H), 1.69-1.80 (m, 2H), 1.63-1.68 (m, 3H), 1.55 (br d, J=12.4 Hz, 2H), 1.41-1.46 (m, 3H). ¹⁹F NMR (377 MHz, CDCl₃) δ −66.73 (s, 3F).

### Example 3-053:4-(3-(4-Acryloylpiperazin-1-yl)azetidin-1-yl)-6-(2,4-dimethyl-1-oxa-8-azaspiro[4.5]dec-3-en-8-yl)-2-(trifluoromethyl)nicotinonitrile

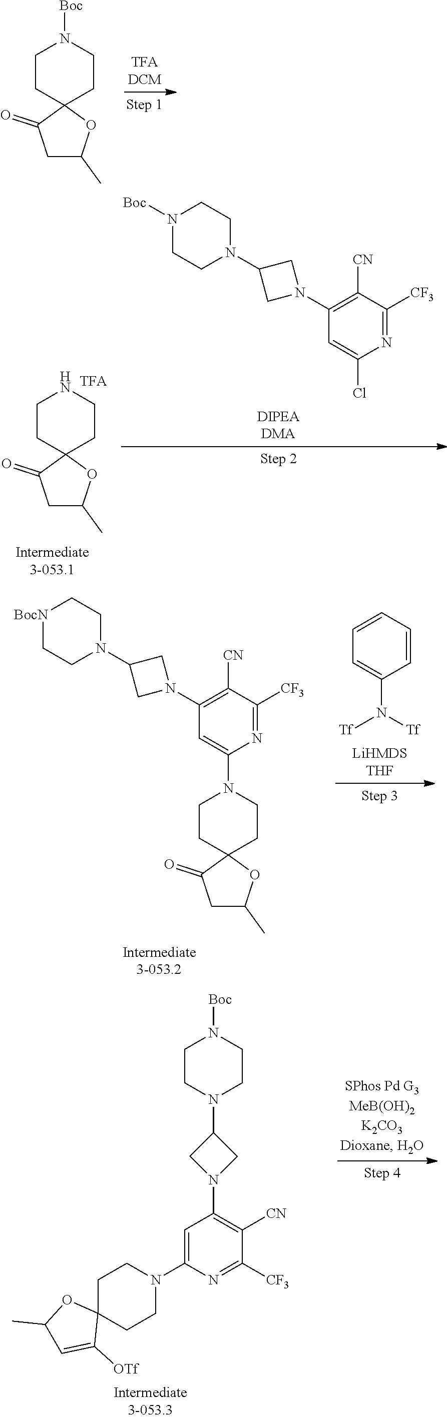

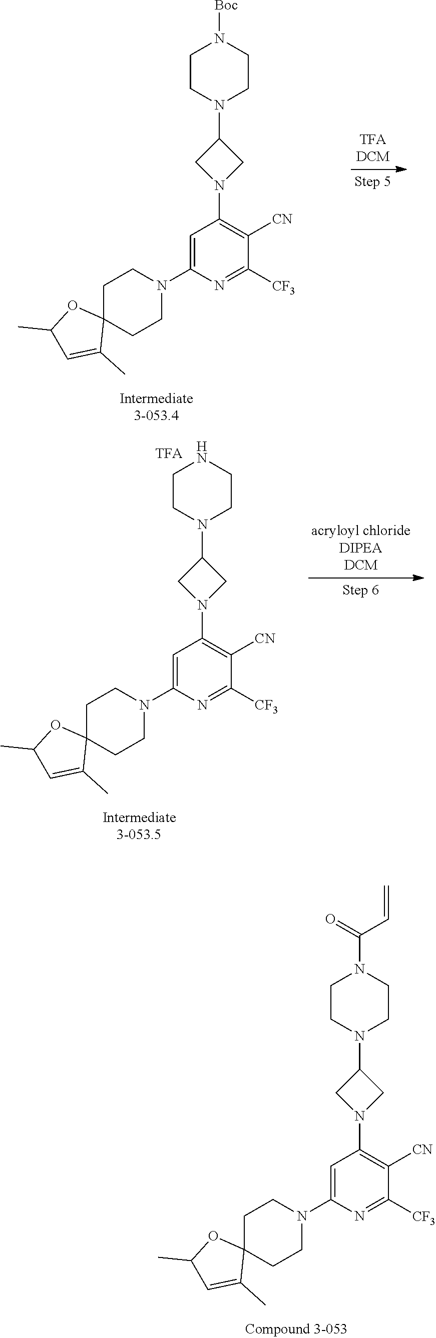 [SMILES: CC1=CC(C)OC12CCN(c1cc(N3CC(N4CCN(C(=O)OC(C)(C)C)CC4)C3)c(C#N)c(C(F)(F)F)n1)CC2 ; CC1=CC(C)OC12CCN(c1cc(N3CC(N4CCNCC4)C3)c(C#N)c(C(F)(F)F)n1)CC2 ; C=CC(=O)N1CCN(C2CN(c3cc(N4CCC5(CC4)OC(C)C=C5C)nc(C(F)(F)F)c3C#N)C2)CC1]

Step 1: 2-Methyl-1-oxa-8-azaspiro[4.5]decan-4-one, Intermediate 3-053.1. tert-Butyl 2-methyl-4-oxo-1-oxa-8-azaspiro[4.5]decane-8-carboxylate (301 mg, 1.12 mmol), DCM (5.6 mL), and TFA (1.67 mL, 22.4 mmol) were stirred at rt for 60 min, then concentrated to provide Intermediate 3-053.1 as a TFA salt which was used in the next step. MS (ESI) m/z: 170.2 (M+H)⁺.

Step 2: tert-Butyl 4-(1-(3-cyano-6-(2-methyl-4-oxo-1-oxa-8-azaspiro[4.5]decan-8-yl)-2-(trifluoromethyl)pyridin-4-yl)azetidin-3-yl)piperazine-1-carboxylate, Intermediate 3-053.2. To the crude material from Step 1 was added DIPEA (2.0 mL, 11.2 mmol) in DMA (5.6 mL) at rt. The reaction mixture was stirred for 10 min at rt, and tert-butyl 4-(1-(6-chloro-3-cyano-2-(trifluoromethyl)pyridin-4-yl)azetidin-3-yl)piperazine-1-carboxylate (448 mg, 1.01 mmol) was added. The reaction mixture was then heated at 86° C. for 24 h. The reaction was quenched by 10 mL sat. aq. NH₄Cl solution and EtOAc (10 mL). The organic phase was washed with H₂O and NH₄Cl sat. aq. solution (2×), and the combined aqueous phase was extracted with EtOAc (48 mL). The organic phase was combined and washed with 8 mL brine, dried over Na₂SO₄, and concentrated. The crude material was purified by silica gel chromatography, eluting with a gradient of 10-90% EtOAc in heptane, to provide Intermediate 3-053.2. MS (ESI) m/z: 579.2 (M+H)⁺.

Step 3: tert-Butyl 4-(1-(3-cyano-6-(2-methyl-4-(((trifluoromethyl)sulfonyl)oxy)-1-oxa-8-azaspiro[4.5]dec-3-en-8-yl)-2-(trifluoromethyl)pyridin-4-yl)azetidin-3-yl)piperazine-1-carboxylate, Intermediate 3-053.3. N-phenyl bis-trifluoromethane sulfonimide (511 mg, 1.43 mmol) and Intermediate 3-053.2 (690 mg, 1.19 mmol) were combined, and the reaction vessel was evacuated and backfilled with N₂. To the reaction mixture was added THF (4 mL) and the reaction mixture was cooled to 0° C. To the reaction mixture was added LiHMDS (1.0 M in THF, 1.43 mL, 1.43 mmol) and the reaction mixture was stirred at rt for 16 h. The reaction mixture was quenched by the addition of sat. aq. NH₄Cl and extracted with EtOAc. The organic extracts were washed with brine, dried over Na₂SO₄, filtered, and concentrated in vacuo. The crude material was purified by silica gel chromatography, eluting with a gradient of 0-40% EtOAc in heptane, to provide Intermediate 3-053.3. MS (ESI) m/z: 711.2 (M+H)⁺.

Step 4: tert-Butyl 4-(1-(3-cyano-6-(2,4-dimethyl-1-oxa8-azaspiro[4.5]dec-3-en-8-yl)-2-(trifluoromethyl)pyridin-4-yl)azetidin-3-yl)piperazine-1-carboxylate, Intermediate 3-053.4. SPhos Pd G3 (24.4 mg, 0.028 mmol), K₂CO₃ (78 mg, 0.56 mmol), methylboronic acid (67 mg, 1.13 mmol), and Intermediate 3-053.3 (200 mg, 0.28 mmol) were combined, and the reaction vessel was evacuated and backfilled with N₂. To the reaction mixture was added 1,4-dioxane (3 mL) and H₂O (0.3 mL) and the reaction mixture was heated to 100° C. and stirred for 2 h. The reaction mixture was filtered through Na₂SO₄ and concentrated in vacuo. The crude material was purified by silica gel chromatography, eluting with a gradient of 0-40% EtOAc in heptane, to provide Intermediate 3-053.4. MS (ESI) m/z: 577.2 (M+H)⁺.

Step 5: 6-(2,4-Dimethyl-1-oxa-8-azaspiro[4.5]dec-3-en-8-yl)-4-(3-(piperazin-1-yl)azetidin-1-yl)-2-(trifluoromethyl)nicotinonitrile (TFA salt), Intermediate 3-053.5. To a solution of Intermediate 3-053.4 (139 mg, 0.24 mmol) in DCM (3 mL) was added TFA (0.5 mL, 0.24 mmol) and the reaction mixture was stirred for 30 min at rt. The reaction mixture was concentrated to provide Intermediate 3-053.5, as a TFA salt. MS (ESI) m/z: 477.2 (M+H)⁺.

Step 6:4-(3-(4-Acryloylpiperazin-1-yl)azetidin-1-yl)-6-(2,4-dimethyl-1-oxa-8-azaspiro[4.5]dec-3-en-8-yl)-2-(trifluoromethyl)nicotinonitrile, Compound 3-053. Intermediate 3-053.5 (TFA salt) was redissolved in DCM (3 mL). To the reaction mixture was added DIPEA (0.42 mL, 2.41 mmol) and acryloyl chloride (0.2 M in DCM, 1.20 mL, 0.24 mmol) and the reaction mixture was stirred at rt for 30 min. The reaction mixture was quenched by the addition of sat. aq. NaHCO₃ and extracted with DCM. The organic extracts were washed with sat. aq. NH₄Cl, dried over Na₂SO₄, and concentrated in vacuo. The crude material was purified by silica gel chromatography, eluting with a gradient of 0-40% EtOAc/EtOH (3:1) in heptane. The material was further purified by reverse-phase preparative HPLC using 0.1% TFA in ACN/H₂O as eluent, with a gradient of 10-100% over 15 min to provide Compound 3-053. MS (ESI) m/z: 531.2 (M+H)¹. 1H NMR (400 MHz, CDCl₃) δ 6.48-6.62 (m, 1H), 6.19-6.37 (m, 1H), 5.66-5.76 (m, 1H), 5.40-5.43 (m, 1H), 5.36-5.40 (m, 1H), 4.76-4.89 (m, 1H), 4.32-4.41 (m, 2H), 4.23-4.32 (m, 2H), 4.06-4.14 (m, 2H), 3.56-3.81 (m, 4H), 3.24-3.37 (m, 3H), 2.35-2.50 (m, 4H), 1.78-1.87 (m, 1H), 1.67-1.76 (m, 1H), 1.63-1.66 (m, 3H). 1.45-1.53 (m, 2H), 1.23-1.28 (m, 3H). ¹⁹F NMR (376 MHz, CDCl₃) δ −66.76 (s, 3F).

### Examples 3-054-1 and 3-054-2: (R)-4-(3-(4-Acryloylpiperazin-1-yl)azetidin-1-yl)-6-(2,4-dimethyl-1-oxa-8-azaspiro[4.5]dec-3-en-8-yl)-2-(trifluoromethyl)nicotinonitrile; (S)-4-(3-(4-Acryloylpiperazin-1-yl)azetidin-1-yl)-6-(2,4-dimethyl-1-oxa-8-azaspiro[4.5]dec-3-en-8-yl)-2-(trifluoromethyl)nicotinonitrile

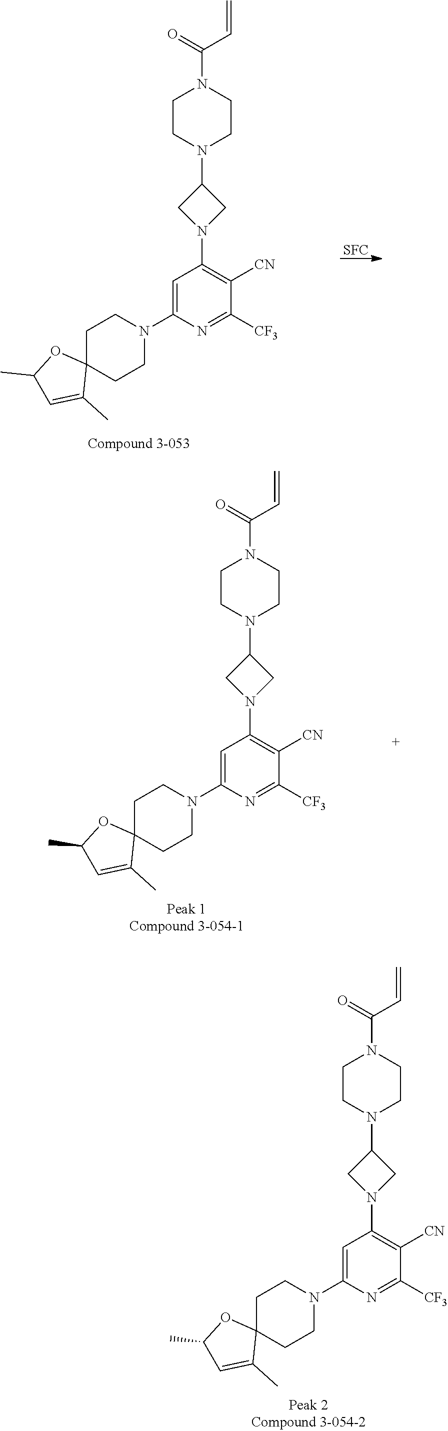 [SMILES: C=CC(=O)N1CCN(C2CN(c3cc(N4CCC5(CC4)OC(C)C=C5C)nc(C(F)(F)F)c3C#N)C2)CC1 ; "SFC" ; C=CC(=O)N1CCN(C2CN(c3cc(N4CCC5(CC4)O[C@H](C)C=C5C)nc(C(F)(F)F)c3C#N)C2)CC1 ; C=CC(=O)N1CCN(C2CN(c3cc(N4CCC5(CC4)O[C@@H](C)C=C5C)nc(C(F)(F)F)c3C#N)C2)CC1]

The stereoisomers of Compound 3-053 were purified via SFC using a Chiralpak AZ, 21×250 mm, 5 μm, column with a mobile phase of 40% MeOH and a flowrate of 100 mL/min. The first eluting peak was arbitrarily assigned as (R)-4-(3-(4-acryloylpiperazin-1-yl) azetidin-1-yl)-6-(2,4-dimethyl-1-oxa-8-azaspiro[4.5]dec-3-en-8-yl)-2-(trifluoromethyl)nicotinonitrile (Compound 3-054-1). MS (ESI) m/z: 531.2 (M+H)⁺. ¹H NMR (400 MHz, CDCl₃) δ 6.42-6.73 (m, 1H), 6.21-6.38 (m, 1H), 5.66-5.78 (m, 1H), 5.40-5.44 (m, 1H), 5.38-5.40 (m, 1H), 4.70-4.91 (m, 1H), 4.32-4.40 (m, 2H), 4.27 (br d, J=12.8 Hz, 2H), 4.00-4.16 (m, 2H), 3.57-3.82 (m, 4H), 3.23 (br d, J=8.8 Hz, 3H), 2.31-2.52 (m, 4H), 1.76-1.88 (m, 1H), 1.66-1.76 (m, 1H), 1.63-1.66 (m, 3H), 1.44-1.52 (m, 2H), 1.21-1.28 (m, 3H). ¹⁹F NMR (376 MHz, CDCl₃) δ −66.75 (s, 3F).

The second eluting peak was arbitrarily assigned as(S)-4-(3-(4-acryloylpiperazin-1-yl)azetidin-1-yl)-6-(2,4-dimethyl-1-oxa-8-azaspiro[4.5]dec-3-en-8-yl)-2-(trifluoromethyl)nicotinonitrile (Compound 3-054-2). MS (ESI) m/z: 531.2 (M+H)⁺. ¹H NMR (400 MHz, CDCl₃) δ 6.48-6.65 (m, 1H), 6.23-6.39 (m, 1H), 5.63-5.84 (m, 1H), 5.32-5.51 (m, 2H), 4.77-4.94 (m, 1H), 4.32-4.43 (m, 2H), 4.22-4.32 (m, 2H), 4.00-4.17 (m, 2H), 3.53-3.84 (m, 4H), 3.18-3.41 (m, 3H), 2.36-2.49 (m, 4H), 1.76-1.92 (m, 1H), 1.66-1.76 (m, 1H), 1.63-1.66 (m, 3H), 1.45-1.54 (m, 2H), 1.20-1.29 (m, 3H). 1° F. NMR (376 MHz, CDCl₃) δ −66.75 (s, 3F).

### Example 3-055: 1-(4-((2R,3R)-1-(6-(1,4-Dimethyl-1,8-diazaspiro[4.5]dec-3-en-8-yl)-2-(trifluoromethyl)pyrimidin-4-yl)-2-methylazetidin-3-yl)piperazin-1-yl)prop-2-en1-one

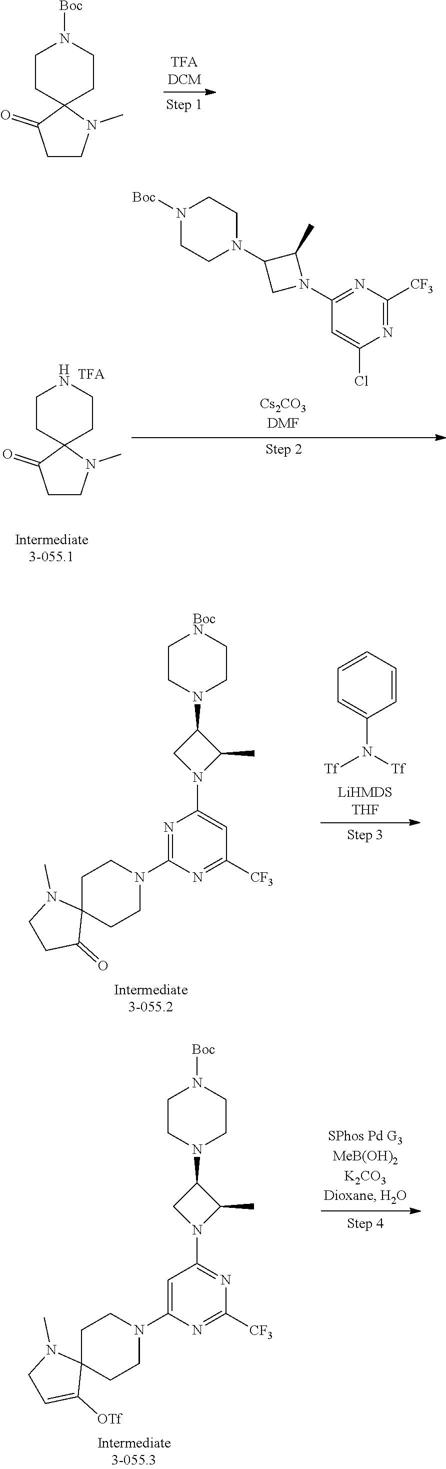

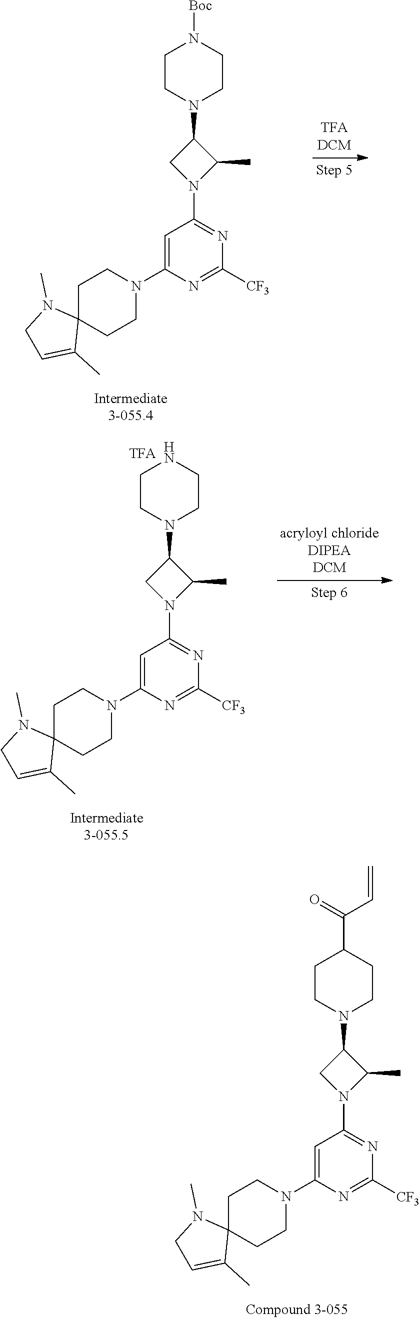 [SMILES: CC1=CCN(C)C12CCN(c1cc(N3C[C@@H](N4CCN(C(=O)OC(C)(C)C)CC4)[C@H]3C)nc(C(F)(F)F)n1)CC2 ; CC1=CCN(C)C12CCN(c1cc(N3C[C@@H](N4CCNCC4)[C@H]3C)nc(C(F)(F)F)n1)CC2 ; C=CC(=O)C1CCN([C@@H]2CN(c3cc(N4CCC5(CC4)C(C)=CCN5C)nc(C(F)(F)F)n3)[C@@H]2C)CC1]

Step 1:1-Methyl-1,8-diazaspiro[4.5]decan-4-one, Intermediate 3-055.1. To a solution of tert-butyl 1-methyl-4-oxo-1,8-diazaspiro[4.5]decane-8-carboxylate (500 mg, 1.86 mmol) in DCM (5 mL) was added TFA (1 mL) and the reaction mixture was stirred at rt for 30 min. The reaction mixture was concentrated in vacuo to provide Intermediate 3-055.1 as a TFA salt, which was used without further purification. MS (ESI) m/z: 169.2 (M+H)⁺.

Step 2: tert-Butyl 4-((2R,3R)-2-methyl-1-(6-(1-methyl-4-oxo-1,8-diazaspiro[4.5]decan-8-yl)-2-(trifluoromethyl)pyrimidin-4-yl)azetidin-3-yl)piperazine-1-carboxylate, Intermediate 3-055.2. To the crude material from Step 1 was added Cs₂CO₃ (1.82 g, 5.59 mmol), ter-butyl 4-((2R,3R)-1-(6-chloro-2-(trifluoromethyl)pyrimidin-4-yl)-2-methylazetidin-3-yl)piperazine-1-carboxylate (812 mg, 1.86 mmol), and DMF (6 mL) and the reaction mixture was heated to 100° C. and stirred 16 h. The reaction mixture was diluted with H₂O and extracted with EtOAc. The organic extracts were washed with brine, filtered through Na₂SO₄, and concentrated in vacuo. The crude material was purified by silica gel chromatography, eluting with a gradient of 0-40% EtOAc in heptane, to provide Intermediate 3-055.2. MS (ESI) m/z: 568.2 (M+H)⁺.

Step 3: tert-Butyl 4-((2R,3R)-2-methyl-1-(6-(1-methyl4-(((trifluoromethyl)sulfonyl)oxy)-1,8-diazaspiro[4.5]dec-3-en-8-yl)-2-(trifluoromethyl)pyrimidin-4-yl)azetidin-3-yl)piperazine-1-carboxylate, Intermediate 3-055.3. N-Phenyl bis-trifluoromethane sulfonimide (518 mg, 1.45 mmol) and Intermediate 3-055.2 (633 mg, 1.12 mmol) were combined, and the reaction vessel was evacuated and backfilled with N₂. To the reaction mixture was added THF (4 mL) and the reaction mixture was cooled to 0° C. To the reaction mixture was added LiHMDS (1.0 M in THF, 1.45 mL, 1.45 mmol) and the reaction mixture was stirred at rt for 3 h. The reaction mixture was quenched by the addition of sat. aq. NH₄Cl and extracted with EtOAc. The organic extracts were washed with brine, dried over Na₂SO₄, filtered and concentrated in vacuo. The crude material was absorbed onto a plug of silica gel and purified by silica gel chromatography, eluting with a gradient of 0-40% EtOAc in heptane, to provide Intermediate 3-055.3. MS (ESI) m/z: 699.8 (M+H)¹.

Step 4: tert-Butyl 4-((2R,3R)-1-(6-(1,4-dimethyl-1,8-diazaspiro[4.5]dec-3-en-8-yl)-2-(trifluoromethyl)pyrimidin-4-yl)-2-methylazetidin-3-yl)piperazine-1-carboxylate, Intermediate 3-055.4. SPhos Pd G3 (63 mg, 0.073 mmol), K₂CO₃ (202 mg, 1.46 mmol), methylboronic acid (175 mg, 2.92 mmol), and Intermediate 3-055.3 (511 mg, 0.73 mmol) were combined, and the reaction vessel was evacuated and backfilled with N₂. To the reaction mixture was added 1,4-dioxane (4 mL) and H₂O (0.4 mL) and the reaction mixture was heated to 100° C. and stirred for 2 h. The reaction mixture was filtered through a plug of silica and eluted with EtOAc. The silica plug was then flushed with MeOH and the solvent was removed in vacuo to provide Intermediate 3-055.4. MS (ESI) m/z: 566.1 (M+H)⁺.

Step 5: 1,4-Dimethyl-8-(6-((2R,3R)-2-methyl-3-(piperazin-1-yl)azetidin-1-yl)-2-(trifluoromethyl)pyrimidin-4-yl)-1,8-diazaspiro[4.5]dec-3-ene (TFA salt), Intermediate 3-055.5. To a solution of Intermediate 3-055.4 (192 mg, 0.34 mmol) in DCM (3 mL) was added TFA (0.5 mL, 0.34 mmol) and the reaction mixture was stirred for 30 min at rt. The reaction was concentrated to give Intermediate 3-055.5, as a TFA salt that was taken to next step. MS (ESI) m/z: 466.0 (M+H)⁺.

Step 6: 1-(4-((2R,3R)-1-(6-(1,4-Dimethyl-1,8-diazaspiro[4.5]dec-3-en-8-yl)-2-(trifluoromethyl)pyrimidin-4-yl)-2-methylazetidin-3-yl)piperazin-1-yl) prop-2-en1-one, Compound 3-055. The crude material from Step 5 was redissolved in DCM (3 mL). To the reaction mixture was added DIPEA (0.59 mL, 3.39 mmol) and acryloyl chloride (0.2 M in DCM, 1.70 mL, 0.34 mmol) and the reaction mixture was stirred at rt for 30 min. The reaction mixture was quenched by the addition of sat. aq. NaHCO₃ and extracted with DCM. The organic extracts were washed with sat. aq. NH₄Cl, dried over Na₂SO₄, and concentrated in vacuo. The crude material was purified by silica gel chromatography, eluting with 100% EtOAc, then eluted with 2M NH₃ in MeOH, The resulting material was purified again by SFC with a gradient of 10-30% MeOH in liquid CO₂. (Column: DEAP 30×150 mm flow rate: 100 mL/min), to provide Compound 3-055. MS (ESI) m/z: 520.2 (M+H)⁺. ¹H NMR (400 MHz, CDCl₃) δ 6.47-6.66 (m, 1H), 6.22-6.35 (m, 1H), 5.71 (dd, J=10.7, 1.9 Hz, 1H). 5.38-5.52 (m, 1H), 5.03-5.17 (m, 1H), 4.46-4.66 (m, 1H), 3.90-4.04 (m, 2H), 3.55-3.81 (m, 8H), 3.37-3.45 (m, 2H), 3.22-3.33 (m, 1H), 2.45 (s, 3H), 2.36 (t, J=4.9 Hz, 4H), 1.84-1.96 (m, 2H), 1.80 (d, J=1.9 Hz, 3H), 1.67-1.77 (m, 2H), 1.48 (d, J=6.3 Hz, 3H). ¹⁹F NMR (376 MHz, CDCl₃) δ −71.77 (s, 3F).

### Example 3-057: 1-(4-((2R,3R)-1-(2-(Difluoromethyl)-3-methyl-6-(4-methyl-1-oxa-8-azaspiro[4.5]dec-3-en-8-yl)pyridin-4-yl)-2-methylazetidin-3-yl)piperazin-1-yl) prop-2-en-1-one

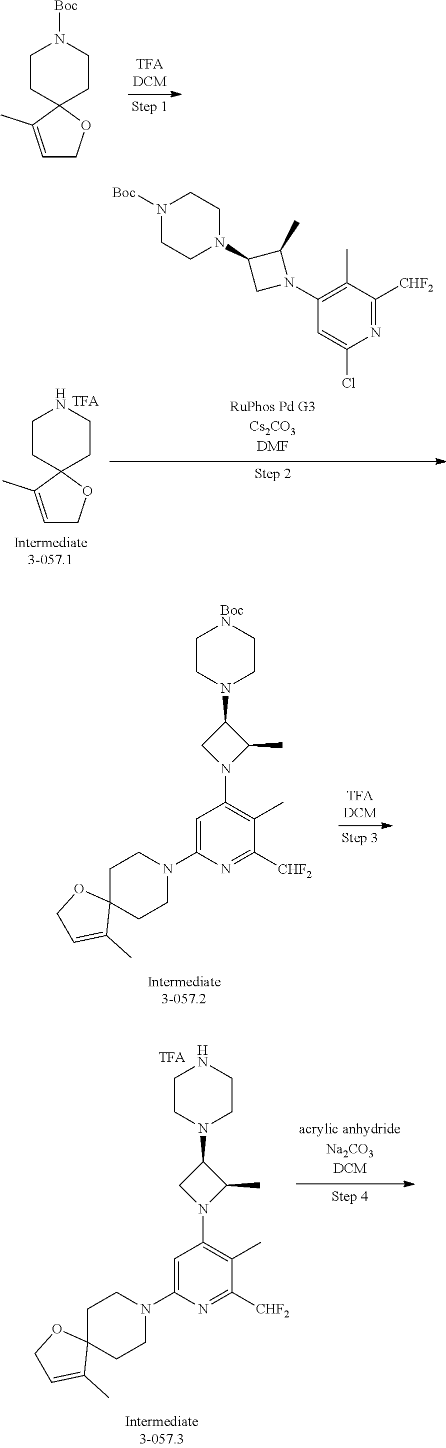 [SMILES: CC1=CCOC12CCN(C(=O)OC(C)(C)C)CC2 ; Cc1c(N2C[C@@H](N3CCN(C(=O)OC(C)(C)C)CC3)[C@H]2C)cc(Cl)nc1C(F)F ; CC1=CCOC12CCNCC2 ; CC1=CCOC12CCN(c1cc(N3C[C@@H](N4CCN(C(=O)OC(C)(C)C)CC4)[C@H]3C)c(C)c(C(F)F)n1)CC2 ; CC1=CCOC12CCN(c1cc(N3C[C@@H](N4CCNCC4)[C@H]3C)c(C)c(C(F)F)n1)CC2]

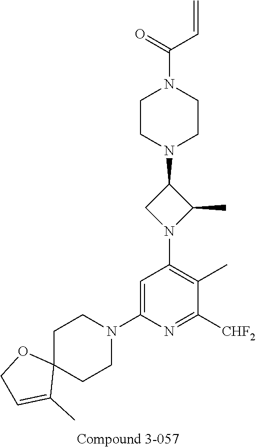 [SMILES: C=CC(=O)N1CCN([C@@H]2CN(c3cc(N4CCC5(CC4)OCC=C5C)nc(C(F)F)c3C)[C@@H]2C)CC1]

Step 1: 4-Methyl-1-oxa-8-azaspiro[4.5]dec-3-ene (TFA salt), Intermediate 3-057.1. tert-Butyl 4-methyl-1-oxa-8-azaspiro[4.5]dec-3-ene-8-carboxylate (300 mg, 1.18 mmol) and TFA (1.8 mL, 23.7 mmol) in DCM (4.7 mL) were stirred for 2 h at rt. The reaction solution was concentrated to provide Intermediate 3-057.1 as a TFA salt which was used in the next step. MS (ESI) m/z: 154.2 (M+H)⁺.

Step 2: tert-Butyl 4-((2R,3R)-1-(2-(difluoromethyl)-3-methyl-6-(4-methyl-1-oxa-8-azaspiro[4.5]dec-3-en-8-yl)pyridin-4-yl)-2-methylazetidin-3-yl)piperazine-1-carboxylate, Intermediate 3-057.2. To the crude material from Step 1 was added Cs₂CO₃ (3.86 g, 11.84 mmol), RuPhos Pd G3 (99 mg, 0.12 mmol), and tert-butyl 4-((2R,3R)-1-(6-chloro-2-(difluoromethyl)-3-methylpyridin-4-yl)-2-methylazetidin-3-yl)piperazine-1-carboxylate (510 mg, 1.18 mmol) in DMF (4.7 mL). The reaction was stirred for 20 h, and quenched by 5 mL H₂O and 10 mL EtOAc. The organic phase was washed by H₂O (3 mL×2), and the combined aqueous phases were extracted with EtOAc (3 mL). The organic phase was combined and washed with brine, dried over Na₂SO₄, and concentrated. The crude material was purified by silica gel chromatography, eluting with 10-50% EtOAc in heptane provide Intermediate 3-057.2. MS (ESI) m/z: 548.0 (M+H)¹.

Step 3: 8-(6-(Difluoromethyl)-5-methyl-4-((2R,3R)-2-methyl-3-(piperazin-1-yl)azetidin-1-yl)pyridin-2-yl)-4-methyl-1-oxa-8-azaspiro[4.5]dec-3-ene (TFA salt), Intermediate 3-057.3. Intermediate 3-057.2 (76 mg, 0.14 mmol) and TFA (207 μL, 2.78 mmol) in DCM (1.4 mL) were stirred at rt for 2 h. The mixture was concentrated to provide Intermediate 3-057.3 as a TFA salt, which was used in the next step. MS (ESI) m/z: 448.0 (M+H)⁺.

Step 4: 1-(4-((2R,3R)-1-(2-(Difluoromethyl)-3-methyl-6-(4-methyl-1-oxa-8-azaspiro[4.5]dec-3-en-8-yl)pyridin-4-yl)-2-methylazetidin-3-yl)piperazin-1-yl)prop-2-en-1-one, Compound 3-057. To the crude material from Step 3 was added Na₂CO₃ (10% in H₂O, 2.9 mL, 2.78 mmol) followed by acrylic anhydride (0.2 M in DCM, 0.70 mL, 0.14 mmol) at 0° C. The reaction was stirred for 15 min, then extracted with DCM (2×1 mL). The organic phase was dried through Na₂SO₄ and concentrated. The crude material was purified by silica gel chromatography, eluting with 15-90% EtOAc in heptane, to provide Compound 3-057. MS (ESI) m/z: 502.0 (M+H)⁺. ¹H NMR (400 MHz, CDCl₃) δ 6.58 (dd, J=16.8, 10.6 Hz, 1H), 6.51 (t, J=55.3 Hz, 1H), 6.32 (dd, J=16.7, 1.9 Hz, 1H), 5.79 (s, 1H), 5.73 (dd, J=10.5, 1.9 Hz, 1H), 5.49 (d, J=1.5 Hz, 1H), 4.49-4.59 (m, 3H), 4.19 (ddt, J=39.2, 12.8, 2.2, 2.2 Hz, 2H), 3.80-3.86 (m, 1H), 3.53-3.79 (m, 5H), 3.35 (q, J=7.1 Hz, 1H), 3.09-3.23 (m, 2H), 2.33-2.46 (m, 4H), 2.17 (s, 3H), 1.84 (tdd, J=13.1, 13.1, 8.4, 5.0 Hz, 2H), 1.68 (d, J=1.9 Hz, 3H), 1.53 (br s, 2H), 1.17 (d, J=6.3 Hz, 3H). ¹⁹F NMR (376 MHz, CDCl₃) δ −112.53 (br d, J=54.6 Hz, 2F).

### Example 3-058:4-(3-(4-Acryloylpiperazin-1-yl)azetidin-1-yl)-6-(4-fluoro-1-oxa-8-azaspiro[4.5]decan-8-yl)-2-(trifluoromethyl)nicotinonitrile

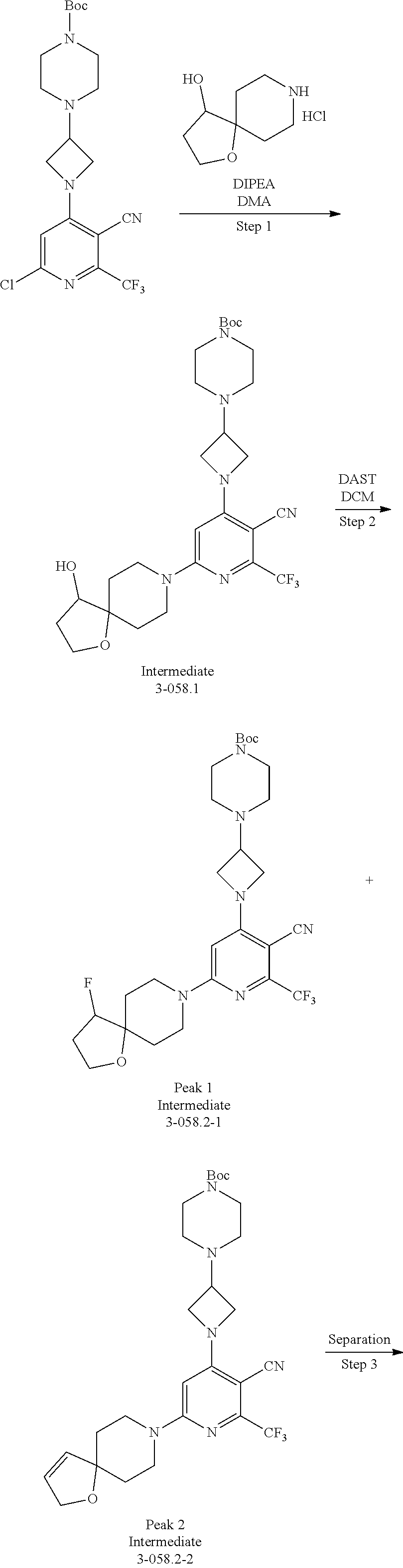 [SMILES: CC(C)(C)OC(=O)N1CCN(C2CN(c3cc(Cl)nc(C(F)(F)F)c3C#N)C2)CC1 ; OC1CCOC12CCNCC2 ; Cl ; CC(C)(C)OC(=O)N1CCN(C2CN(c3cc(N4CCC5(CC4)OCCC5O)nc(C(F)(F)F)c3C#N)C2)CC1 ; CC(C)(C)OC(=O)N1CCN(C2CN(c3cc(N4CCC5(CC4)OCCC5F)nc(C(F)(F)F)c3C#N)C2)CC1 ; CC(C)(C)OC(=O)N1CCN(C2CN(c3cc(N4CCC5(C=CCO5)CC4)nc(C(F)(F)F)c3C#N)C2)CC1]

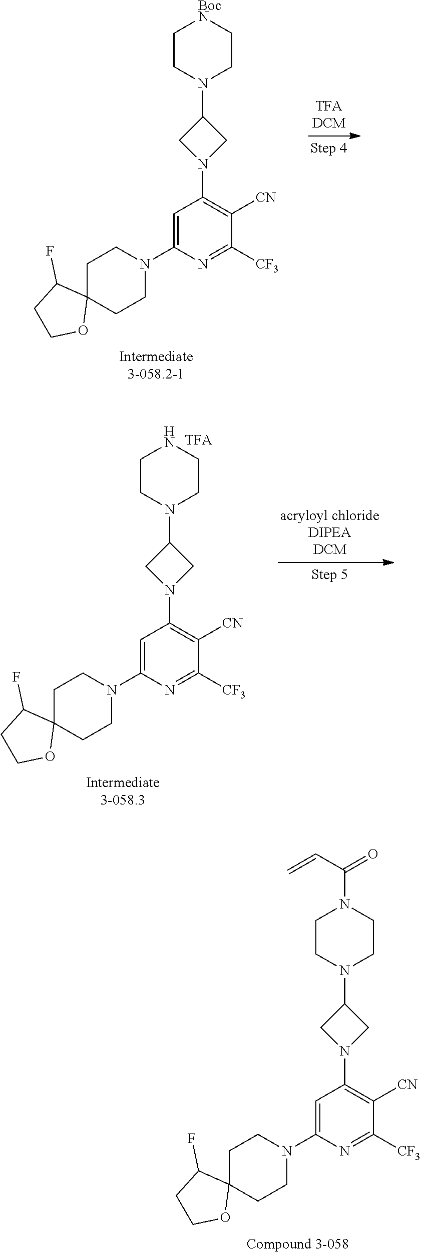 [SMILES: CC(C)(C)OC(=O)N1CCN(C2CN(c3cc(N4CCC5(CC4)OCCC5F)nc(C(F)(F)F)c3C#N)C2)CC1 ; N#Cc1c(N2CC(N3CCNCC3)C2)cc(N2CCC3(CC2)OCCC3F)nc1C(F)(F)F ; C=CC(=O)N1CCN(C2CN(c3cc(N4CCC5(CC4)OCCC5F)nc(C(F)(F)F)c3C#N)C2)CC1]

Step 1: tert-Butyl 4-(1-(3-cyano-6-(4-hydroxy-1-oxa-8-azaspiro[4.5]decan-8-yl)-2-(trifluoromethyl)pyridin-4-yl)azetidin-3-yl)piperazine-1-carboxylate, Intermediate 3-058.1, 1-Oxa-8-azaspiro[4.5]decan-4-ol hydrochloride (200 mg, 1.03 mmol), DIPEA (541 μL, 3.10 mmol), tert-butyl 4-(1-(6-chloro-3-cyano-2-(trifluoromethyl)pyridin-4-yl)azetidin-3-yl)piperazine-1-carboxylate (414 mg, 0.93 mmol), and DMA (5.2 mL) were combined and degassed with N₂ for 15 min, then the reaction mixture was stirred at 95° C. for 14 h. The reaction was quenched by sat. aq. NH₄Cl (5 mL) and EtOAc (5 mL). The organic phase was washed by H₂O (3 mL×2), and the combined aqueous phases were extracted with 2 mL EtOAc. The organic phases were combined and washed with brine, dried over Na₂SO₄, and concentrated. The crude material was purified by silica gel chromatography, eluting with 0-5% MeOH in DCM, to provide Intermediate 3-058.1. MS (ESI) m/z: 567.2 (M+H)⁺.

Step 2: tert-Butyl 4-(1-(3-cyano-6-(4-fluoro-1-oxa-8-azaspiro[4.5]decan-8-yl)-2-(trifluoromethyl)pyridin-4-yl)azetidin-3-yl)piperazine-1-carboxylate, Intermediate 3-058.2-1; tert-butyl 4-(1-(3-cyano-6-(1-oxa-8-azaspiro[4.5]dec-3-en-8-yl)-2-(trifluoromethyl)pyridin-4-yl)azetidin-3-yl)piperazine-1-carboxylate, Intermediate 3-058.2-2. Intermediate 3-058.1 (100 mg, 0.18 mmol), DCM (1.8 mL), and DAST (35.0 μL, 0.265 mmol) were combined at 0° C., and stirred for 16 h. The reaction mixture was then heated to 40° C. for 40 h. The reaction was quenched by sat. aq. NaHCO₃ (10 mL) at 0° C., extracted with DCM (3×2 mL), washed with brine, dried through Na₂SO₄, and concentrated to provide crude material containing Intermediate 3-058.2-1 and Intermediate 3-058.2-2. MS (ESI) m/z: 569.0 (M+H)⁺.

Step 3. The crude product of Step 2 was purified by prep HPLC eluting with a gradient of (0.1% NH₄OH) H₂O in ACN to provide a first and a second peak.

The first eluting peak was identified as Intermediate 3-058.2-1. MS (ESI) m/z: 569.2 (M+H)⁺. The second eluting peak was identified as Intermediate 3-058.2-2. MS (ESI) m/z: 549.2 (M+H)⁺.

Step 4: 6-(4-Fluoro-1-oxa-8-azaspiro[4.5]decan-8-yl)-4-(3-(piperazin-1-yl)azetidin-1-yl)-2-(trifluoromethyl)nicotinonitrile (TFA salt), Intermediate 3-058.3. Intermediate 3-058.2-1 (7 mg, 0.012 mmol) and TFA (18 μL, 0.246 mmol) in DCM (200 μL) were stirred at rt for 1 h. The reaction mixture was concentrated to provide Intermediate 3-058.3, as a TFA salt which was used in the next step. MS (ESI) m/z: 469.2 (M+H)⁺.

Step 5: 4-(3-(4-Acryloylpiperazin-1-yl)azetidin-1-yl)-6-(4-fluoro-1-oxa-8-azaspiro[4.5]decan-8-yl)-2-(trifluoromethyl)nicotinonitrile, Compound 3-058. To the crude mixture of Step 4 was added 0.2 mL DCM followed by DIPEA (21 μL, 0.12 mmol) and acryloyl chloride (0.2 M in DCM, 62 μL, 0.012 mmol) at rt and stirred for 15 min. The reaction solution was quenched and washed by sat. aq. NH₄Cl and brine. The organic phase was dried through Na₂SO₄, and concentrated. The crude material was purified by prep TLC, to provide Compound 3-058. MS (ESI) m/z: 523.2 (M+H)⁺. ¹H NMR (400 MHz, CDCl₃) δ 6.58 (dd, J=16.8, 10.6 Hz, 1H), 6.29-6.36 (m, 1H), 5.73 (br d, J=10.7 Hz, 1H), 5.41 (s, 1H), 4.69-4.94 (m, 1H), 4.30-4.45 (m, 2H), 3.91-4.16 (m, 6H), 3.75 (br s, 2H), 3.63 (br s, 2H), 3.40-3.56 (m, 2H), 3.27-3.36 (m, 1H), 2.21-2.47 (m, 6H), 1.74-1.98 (m, 2H), 1.56-1.65 (m, 2H). ¹⁹F NMR (376 MHz, CDCl₃) δ −66.76 (s, 3F), −189.72-−187.02 (m, 1F).

### Example 3-059:4-(3-(4-Acryloylpiperazin-1-yl)azetidin-1-yl)-6-(1-oxa-8-azaspiro[4.5]dec-3-en-8-yl)-2-(trifluoromethyl)nicotinonitrile

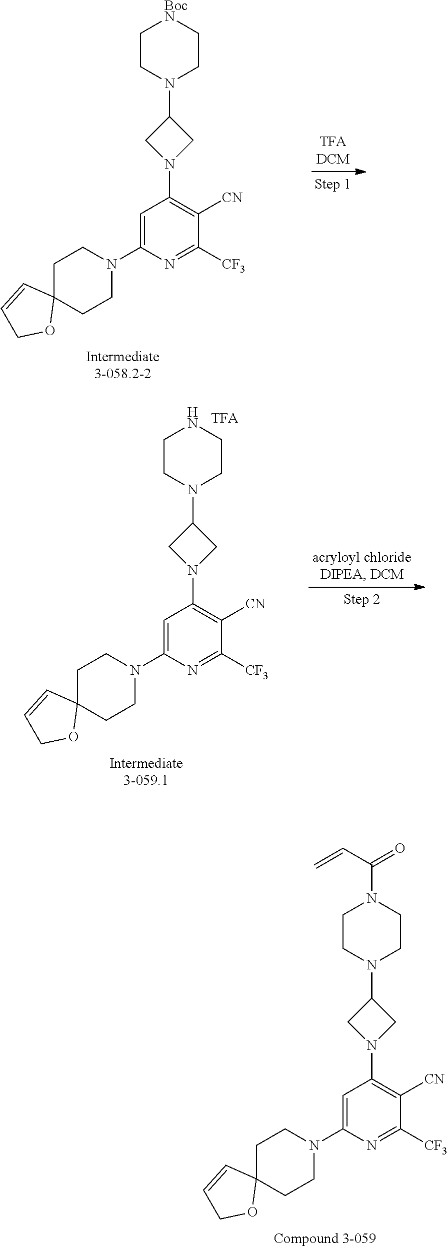 [SMILES: CC(C)(C)OC(=O)N1CCN(C2CN(c3cc(N4CCC5(C=CCO5)CC4)nc(C(F)(F)F)c3C#N)C2)CC1 ; N#Cc1c(N2CC(N3CCNCC3)C2)cc(N2CCC3(C=CCO3)CC2)nc1C(F)(F)F ; C=CC(=O)N1CCN(C2CN(c3cc(N4CCC5(C=CCO5)CC4)nc(C(F)(F)F)c3C#N)C2)CC1]

Step 1: 4-(3-(Piperazin-1-yl)azetidin-1-yl)-6-(1-oxa-8-azaspiro[4.5]dec-3-en-8-yl)-2-(trifluoromethyl)nicotinonitrile (TFA salt), Intermediate 3-059.1. Intermediate 3-058.2-2 (13.5 mg, 0.025 mmol) and TFA (37 μL, 0.49 mmol) in DCM (0.25 mL) were stirred at rt for 14 h. The reaction mixture was concentrated to provide Intermediate 3-059.1, as a TFA salt, which was used in the next step. MS (ESD) m/z: 449.2 (M+H)⁺.

Step 2: 4-(3-(4-Acryloylpiperazin-1-yl)azetidin-1-yl)-6-(1-oxa-8-azaspiro[4.5]dec-3-en-8-yl)-2-(trifluoromethyl)nicotinonitrile, Compound 3-059. To the product of Step 1 was added 0.2 mL DCM followed by DIPEA (43 μL, 0.25 mmol) and acryloyl chloride (0.2 M in DCM, 0.12 mL, 0.025 mmol) at rt and stirred for 15 min. The reaction solution was quenched and washed by 1 mL NH₄Cl sat. aq. solution and brine. The organic phase was dried through Na₂SO₄ and concentrated. The crude material was purified by prep TLC to provide Compound 3-059. MS (ESI) m/s: 503.2 (M+H)⁺. ¹H NMR (400 MHz, CDCl₃) δ 6.57 (dd, J=16.8, 10.6 Hz, 1H), 6.32 (br d, J=16.9 Hz, 1H), 5.94 (dd, J=6.2, 1.4 Hz, 1H), 5.68-5.80 (m, 2H), 5.41 (s, 1H), 4.70 (s, 2H), 4.38 (br t, J=7.4 Hz, 2H), 3.98-4.17 (m, 4H), 3.56-3.84 (m, 4H), 3.40-3.53 (m, 2H), 3.23-3.36 (m, 1H), 2.43 (br t, J=4.5 Hz, 4H), 1.66-1.82 (m, 4H). ¹⁹F NMR (376 MHz, CDCl₃) δ −66.76 (s, 3F).

### Example 3-060:1-(4-((2R,3R)-1-(6-(4,6-Dimethyl-1-oxa-8-azaspiro[4.5]dec-3-en-8-yl)-2-(trifluoromethyl)pyrimidin-4-yl)-2-methylazetidin-3-yl)piperazin-1-yl) prop-2-en-1-one

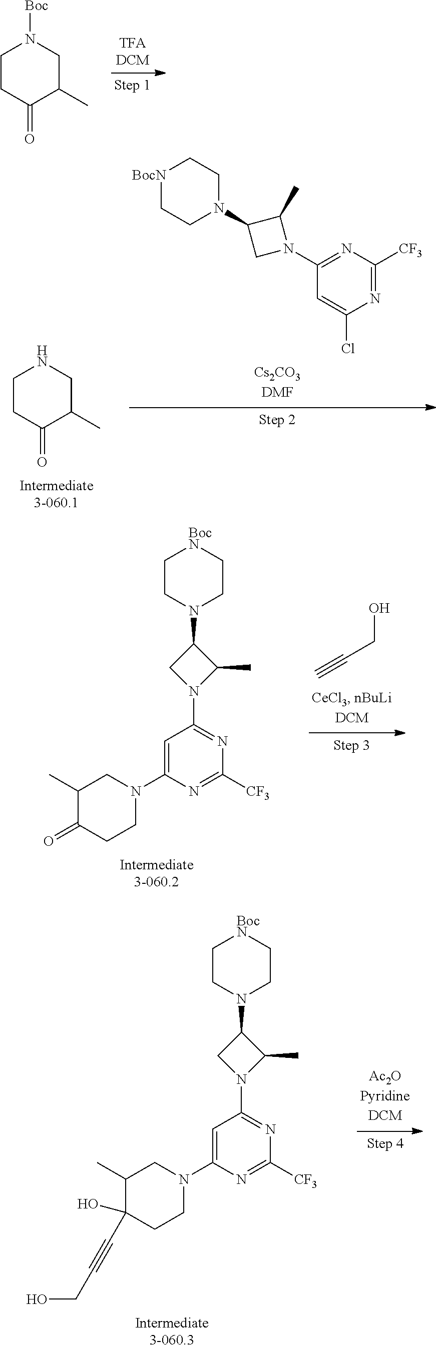

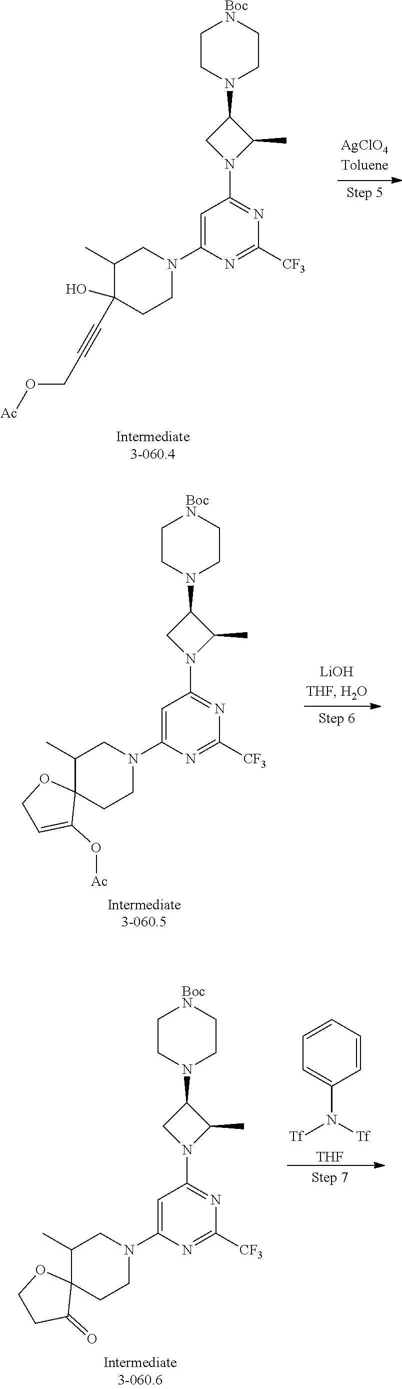

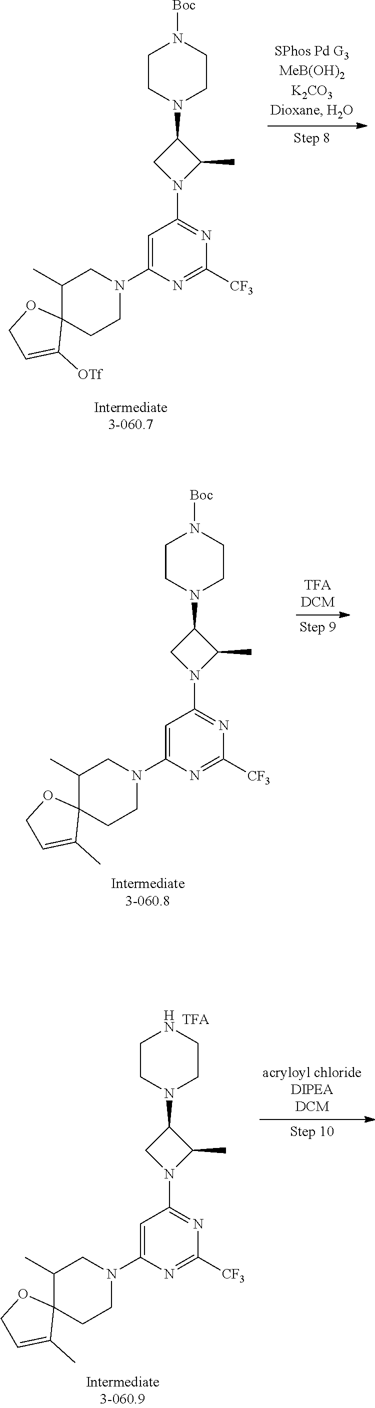 [SMILES: CC1CN(c2cc(N3C[C@@H](N4CCN(C(=O)OC(C)(C)C)CC4)[C@H]3C)nc(C(F)(F)F)n2)CCC12OCC=C2OS(=O)(=O)C(F)(F)F ; CC1=CCOC12CCN(c1cc(N3C[C@@H](N4CCN(C(=O)OC(C)(C)C)CC4)[C@H]3C)nc(C(F)(F)F)n1)CC2C ; CC1=CCOC12CCN(c1cc(N3C[C@@H](N4CCNCC4)[C@H]3C)nc(C(F)(F)F)n1)CC2C]

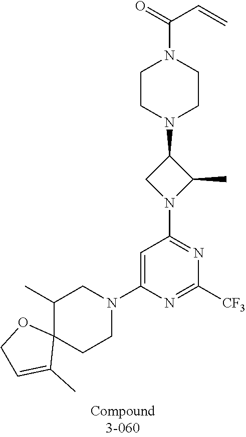 [SMILES: C=CC(=O)N1CCN([C@@H]2CN(c3cc(N4CCC5(OCC=C5C)C(C)C4)nc(C(F)(F)F)n3)[C@@H]2C)CC1]

Step 1: 3-Methylpiperidin-4-one, Intermediate 3-060.1. To a solution of tert-butyl 3-methyl-4-oxopiperidine-1-carboxylate (2.0 g, 9.38 mmol) in DCM (3 mL) was added TFA (1.5 mL, 9.38 mmol) and the reaction mixture was stirred at rt for 1 h. The reaction mixture was concentrated in vacuo and used without further purification. MS (ESI) m/s: 114.4 (M+H)⁺.

Step 2: tert-Butyl 4-((2R,3R)-2-methyl-1-(6-(3-methyl-4-oxopiperidin-1-yl)-2-(trifluoromethyl)pyrimidin-4-yl)azetidin-3-yl)piperazine 1-carboxylate, Intermediate 3-060.2. To the crude product from Step 1 was added Cs₂CO₃ (9.17 g. 28.1 mmol), tert-butyl 4-((2R,3R)-1-(6-chloro-2-(trifluoromethyl)pyrimidin-4-yl)-2-methylazetidin-3-yl)piperazine-1-carboxylate (4.09 g, 9.38 mmol), and DMF (5 mL) and the reaction mixture was heated to 100° C. and stirred 16 h. The reaction mixture was diluted with brine and extracted with EtOAc (2×). The organic extracts were washed with brine, dried over Na₂SO₄, filtered, and concentrated in vacuo. The crude material purified by silica gel chromatography, eluting with a gradient of 0-40% EtOAc in heptane followed by reverse phase chromatography, with ACN/H₂O with 1% TFA, with a gradient of 0-100% over 20 min. The product fractions were washed with sat. aq. Na₂CO₃, extracted with EtOAc, washed with brine, dried over Na₂SO₄, and concentrated in vacuo to provide Intermediate 3-060.2. MS (ESI) m/z: 513.4 (M+H)⁺.

Step 3: tert-Butyl 4-((2R,3R)-1-(6-(4-hydroxy-4-(3-hydroxyprop-1-yn-1-yl)-3-methylpiperidin-1-yl)-2-(trifluoromethyl)pyrimidin-4-yl)-2-methylazetidin-3-yl)piperazine-1-carboxylate, Intermediate 3-060.3. To a −78° C. solution of propargyl alcohol (0.24 mL, 3.96 mmol) in THF (3.6 mL) was added n-BuLi (2.5 M in hexanes, 3.17 mL, 7.91 mmol). To the reaction mixture was added 4 mL of THE and the reaction mixture was stirred for 40 min at this temperature, and 20 min at 0° C. The reaction mixture was cooled back to −78° C. and added a solution of Intermediate 3-060.2 (922 mg, 1.80 mmol) and cerium (III) chloride (0.089 g, 0.36 mmol) in THF (1 mL) that was pre-stirred for 30 min. The reaction mixture was stirred at −78° C. for 30 min and then warmed to rt and stirred for 4 h. The reaction mixture was quenched by the addition of 1M aq HCl and extracted with EtOAc (2×). The organic extracts were washed with brine, dried over Na₂SO₄, filtered, and concentrated in vacuo. The crude material purified by silica gel chromatography, eluting with a gradient of 0-60% EtOAc/EtOH (3:1) in heptane, to provide Intermediate 3-060.3. MS (ESI) m/z: 569.2 (M+H)⁺.

Step 4: tert-Butyl 4-((2R,3R)-1-(6-(4-(3-acetoxyprop-1-yn-1-yl)-4-hydroxy-3-methylpiperidin-1-yl)-2-(trifluoromethyl)pyrimidin-4-yl)-2-methylazetidin-3-yl)piperazine-1-carboxylate, Intermediate 3-060.4. To a solution of Intermediate 3-060.3 (456 mg, 0.80 mmol) and pyridine (0.32 mL, 4.01 mmol) in DCM (3 mL) at 0° C. was added acetic anhydride (0.38 mL, 4.01 mmol) and the reaction mixture was stirred at rt for 16 h. The reaction mixture was concentrated in vacuo, redissolved in EtOAc, and washed with sat. aq. NaHCO₃. The organic extracts were dried over a small plug of Na₂SO₄ and concentrated in vacuo. The crude material was purified by silica gel chromatography, eluting with a gradient of 0-40% EtOAc/EtOH (3:1) in heptane, to provide Intermediate 3-060.4. MS (ESI) m/z: 611.2 (M+H)⁺.

Step 5: tert-Butyl 4-((2R,3R)-1-(6-(4-acetoxy-6-methyl-1-oxa-8-azaspiro[4.5]dec3-en-8-yl)-2-(trifluoromethyl)pyrimidin-4-yl)-2-methylazetidin-3-yl)piperazine-1-carboxylate, Intermediate 3-060.5. To a glass vial was added Intermediate 3-060.4 (455 mg, 0.74 mmol) and silver perchlorate (15 mg, 0.074 mmol). The reaction vessel was evacuated and backfilled with N₂ and to the glass vial was added toluene (2 mL). The reaction mixture was heated to 80° C. and stirred 16 h. The reaction mixture was filtered through a plug of silica, eluted with EtOAc/EtOH (3:1), and concentrated in vacuo. The crude material was purified by silica gel chromatography, eluting with a gradient of 0-20% EtOAc/EtOH (3:1) in heptane to provide Intermediate 3-060.5. MS (ESI) m/z: 611.2 (M+H)⁺.

Step 6: tert-Butyl 4-((2R,3R)-2-methyl-1-(6-(6-methyl-4-oxo-1-oxa-8-azaspiro[4.5]decan-8-yl)-2-(trifluoromethyl)pyrimidin-4-yl)azetidin-3-yl)piperazine-1-carboxylate, Intermediate 3-060.6. Intermediate 3-060.5 (87 mg, 0.14 mmol), lithium hydroxide monohydrate (59.6 mg, 1.42 mmol), THF (2 mL) and H₂O (1 mL) were combined, and the reaction mixture was stirred at rt for 1 h. The reaction mixture was diluted with brine and extracted with EtOAc. The organic extracts were dried over Na₂SO₄ and concentrated in vacuo to provide the crude Intermediate 3-060.6 which was used without further purification. MS (ESI) m/z: 569.4 (M+H)⁺.

Step 7: tert-Butyl 4-((2R,3R)-2-methyl-1-(6-(6-methyl-4-(((trifluoromethyl)sulfonyl)oxy)-1-oxa-8-azaspiro[4.5]dec-3-en-8-yl)-2-(trifluoromethyl)pyrimidin-4-yl)azetidin-3-yl)piperazine-1-carboxylate, Intermediate 3-060.7. N-phenyl bis-trifluoromethane sulfonimide (63 mg, 0.18 mmol) and Intermediate 3-060.6 (77 mg, 0.14 mmol) were combined, and the reaction vessel was evacuated and backfilled with N₂. To the reaction mixture was added THF (2 mL) and cooled to 0° C. To the reaction mixture was added LiHMDS (1.0 M in THF, 0.18 mL, 0.18 mmol) and stirred at rt for 3 h. The reaction mixture was quenched by the addition of sat. aq. NH₄Cl and extracted with EtOAc. The organic extracts were washed with brine, dried over Na₂SO₄, filtered, and concentrated in vacuo to provide crude Intermediate 3-060.7, which was used without further purification. MS (ESI) m/z: 701.0 (M+H)⁺.

Step 8: tert-Butyl 4-((2R,3R)-1-(6-(4,6-dimethyl-1-oxa-8-azaspiro[4.5]dec-3-on-8-yl)-2-(trifluoromethyl)pyrimidin-4-yl)-2-methylazetidin-3-yl)piperazine-1-carboxylate, Intermediate 3-060.8. SPhos Pd G3 (11.7 mg, 0.014 mmol), K₂CO₃ (37.5 mg, 0.27 mmol), methylboronic acid (32 mg, 0.54 mmol), and the crude material from Step 7 (95 mg, 0.14 mmol) were combined, and the reaction vessel was evacuated and backfilled with N₂. To the reaction mixture was added 1,4-dioxane (3 mL) and H₂O (0.3 mL) and heated to 100° C. and stirred for 16 h. The reaction mixture was filtered through Na₂SO₄ and concentrated in vacuo. The crude material was purified by silica gel chromatography, eluting with a gradient of 0-40% EtOAc in heptane, to provide Intermediate 3-060.8. MS (ESI) m/z: 567.4 (M+H)⁺.

Step 9: 4,6-Dimethyl-8-(6-((2R,3R)-2-methyl-3-(piperazin-1-yl)azetidin-1-yl)-2-(trifluoromethyl)pyrimidin-4-yl)-1-oxa-8-azaspiro[4.5]dec-3-ene (TFA salt), Intermediate 3-060.9. To a solution of Intermediate 3-060.8 (43.7 mg, 0.077 mmol) in DCM (3 mL) was added TFA (0.5 mL, 0.077 mmol) and the reaction mixture was stirred for 30 min at rt. The reaction mixture was concentrated to provide Intermediate 3-060.9, as a TFA salt which was used in the next step. MS (ESI) m/z: 467.2 (M+H)⁺.

Step 10: 1-(4-((2R,3R)-1-(6-((5R,6S)-4,6-Dimethyl-1-oxa-8-azaspiro[4.5]dec-3-en-8-yl)-2-(trifluoromethyl)pyrimidin-4-yl)-2-methylazetidin-3-yl)piperazin-1-yl) prop-2-en-1-one, Compound 3-060. The crude material from Step 9 was redissolved in DCM (3 mL) and DIPEA (0.13 mL, 0.77 mmol) and acryloyl chloride (0.2 M in DCM, 0.39 mL, 0.077 mmol) were added. The reaction mixture was stirred at rt for 30 min, quenched by the addition of sat. aq. NaHCO₃ and extracted with DCM. The combined organic extracts were washed with sat. aq. NH₄Cl, dried over a small plug of Na₂SO₄, and concentrated in vacuo. The crude material was purified by reverse-phase preparative HPLC with a gradient of 10-100% (0.1% TFA) ACN in H₂O over 15 min. The fractions containing pure product were washed with sat. aq. Na₂CO₃ and extracted with EtOAc. The combined organic extracts were washed with brine, dried over Na₂SO₄, filtered, and concentrated to provide Compound 3-060. The relative stereochemistry between the methyl and the spirocyclic center is cis. MS (ESI) m/z: 521.2 (M+H)⁺. ¹H NMR (400 MHz, CDCl₃) δ 6.56 (dd, J=16.8, 10.6 Hz, 1H), 6.30 (dd, J=16.7, 1.9 Hz, 1H), 5.66-5.77 (m, 1H), 5.48-5.59 (m, 1H), 5.17-5.26 (m, 1H), 4.45-4.70 (m, 3H), 4.04-4.33 (m, 2H), 3.89-4.03 (m, 2H), 3.58-3.79 (m, 4H), 3.34 (s, 2H), 2.81-2.98 (m, 1H), 2.24-2.48 (m, 4H), 1.68-1.83 (m, 2H), 1.63 (d, J=1.7 Hz, 3H), 1.56-1.59 (m, 1H), 1.48 (d, J=6.3 Hz, 3H), 0.76-0.84 (m, 3H). ¹⁹F NMR (376 MHz, CDCl₃) δ −71.76 (s, 3F).

### Example 3-061:1-(4-((2R,3R)-2-Methyl-1-(6-(4-methyl-1-oxa-8-azaspiro[4.5]dec-3-en-8-yl)-2-(trifluoromethyl)pyrimidin-4-yl)azetidin-3-yl)piperazin-1-yl)prop-2-en-1-one

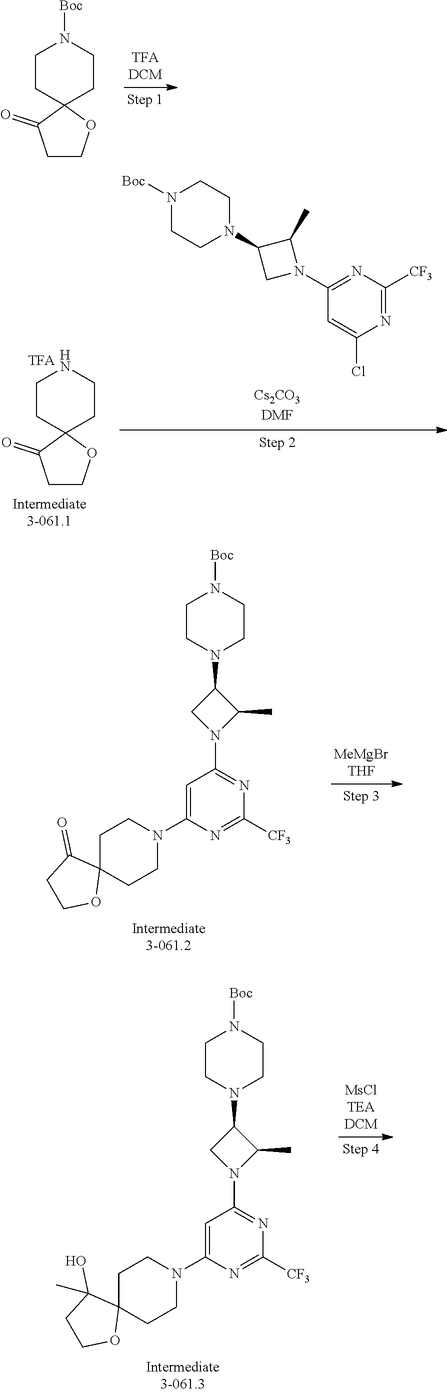 [SMILES: CC(C)(C)OC(=O)N1CCC2(CC1)OCCC2=O ; O=C1CCOC12CCNCC2 ; C[C@@H]1[C@H](N2CCN(C(=O)OC(C)(C)C)CC2)CN1c1cc(Cl)nc(C(F)(F)F)n1 ; C[C@@H]1[C@H](N2CCN(C(=O)OC(C)(C)C)CC2)CN1c1cc(N2CCC3(CC2)OCCC3=O)nc(C(F)(F)F)n1 ; C[C@@H]1[C@H](N2CCN(C(=O)OC(C)(C)C)CC2)CN1c1cc(N2CCC3(CC2)OCCC3(C)O)nc(C(F)(F)F)n1]

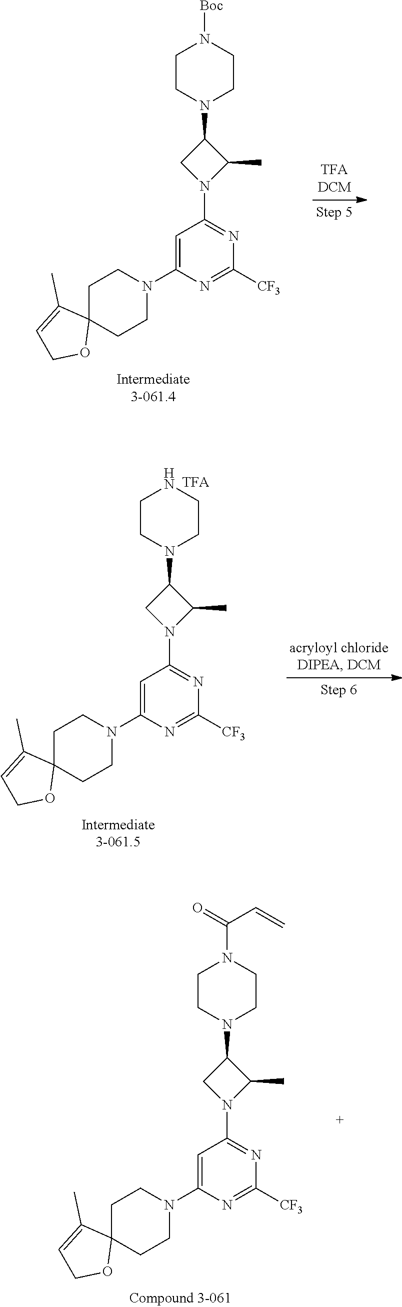 [SMILES: CC1=CCOC12CCN(c1cc(N3C[C@@H](N4CCN(C(=O)OC(C)(C)C)CC4)[C@H]3C)nc(C(F)(F)F)n1)CC2 ; CC1=CCOC12CCN(c1cc(N3C[C@@H](N4CCNCC4)[C@H]3C)nc(C(F)(F)F)n1)CC2 ; C=CC(=O)N1CCN([C@@H]2CN(c3cc(N4CCC5(CC4)OCC=C5C)nc(C(F)(F)F)n3)[C@@H]2C)CC1]

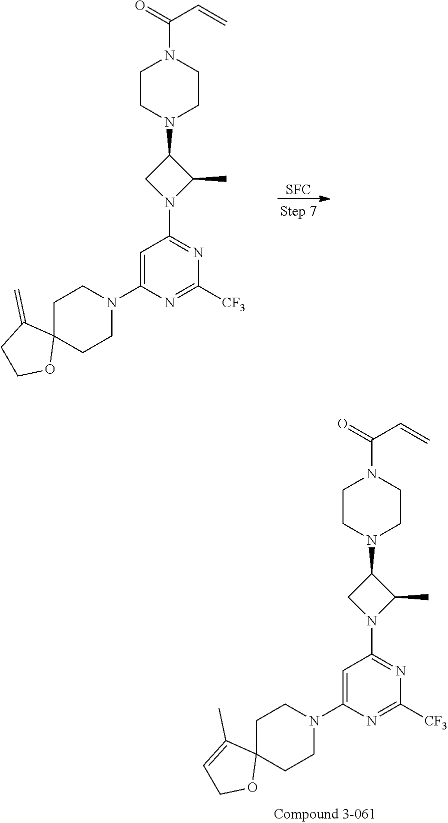

Step 1: 1-Oxa-8-azaspiro[4.5]decan-4-one (TFA salt), Intermediate 3-061.1. To a solution of tert-butyl 4-oxo-1-oxa-8-azaspiro[4.5]decane-8-carboxylate (300 mg, 1.18 mmol) in DCM (3 mL) was added TFA (1 mL, 13 mmol) and the reaction mixture was stirred for 30 min at rt. The reaction mixture was concentrated to give Intermediate 3-061.1 as a TFA salt which was used without further purification. MS (ESI) m/z: 156.2 (M+H)¹.

Step 2: tert-Butyl 4-((2R,3R)-2-methyl-1-(6-(4-oxo-1-oxa-8-azaspiro[4.5]decan-8-yl)-2-(trifluoromethyl)pyrimidin-4-yl)azetidin-3-yl)piperazine-1-carboxylate, Intermediate 3-061.2. To the crude material from Step 1 was added Cs₂CO₃ (1.15 g, 3.53 mmol), tert-butyl 4-((2R,3R)-1-(6-chloro-2-(trifluoromethyl)pyrimidin-4-yl)-2-methylazetidin-3-yl)piperazine-1-carboxylate (512 mg, 1.18 mmol), and DMF (5 mL). The reaction mixture was heated to 100° C. for 6 h. The reaction mixture was diluted with brine and H₂O and extracted with EtOAc. The organic extracts were washed with brine, dried over Na₂SO₄, filtered, and concentrated in vacuo. The crude material was purified by silica gel chromatography, eluting with a gradient of 0-40% EtOAc in heptane, to provide Intermediate 3-061.2. MS (ESI) m/z: 555.4 (M+H)⁺.

Step 3: tert-Butyl 4-((2R,3R)-1-(6-(4-hydroxy-4-methyl-1-oxa-8-azaspiro[4.5]decan-8-yl)-2-(trifluoromethyl)pyrimidin-4-yl)-2-methylazetidin-3-yl)piperazine-1-carboxylate, Intermediate 3-061.3. To a solution of Intermediate 3-061.2 (498 mg, 0.90 mmol) in THF (5 mL) at −78° C. was added methyl magnesium bromide solution (3.0 M in diethyl ether, 0.60 mL, 1.80 mmol) and the reaction mixture was stirred at this temperature for 15 min and then warmed to rt and stirred for an additional 15 min. The reaction mixture was quenched by the addition of sat. aq. NH₄Cl and extracted with EtOAc. The organic extracts were dried over Na₂SO₄ and concentrated in vacuo. The crude material was purified by chromatography, eluting with a gradient of 0-80% EtOAc in heptane, to provide Intermediate 3-061.3. MS (ESI) m/z: 571.2 (M+H)⁺.

Step 4: tert-Butyl 4-((2R,3R)-2-methyl-1-(6-(4-methyl-1-oxa-8-azaspiro[4.5]dec-3-en-8-yl)-2-(trifluoromethyl)pyrimidin-4-yl)azetidin-3-yl)piperazine-1-carboxylate, Intermediate 3-061.4. To a solution of Intermediate 3-061.3 (407 mg, 0.71 mmol) in DCM (3 mL) at 0° C. was added TEA (0.50 mL, 3.6 mmol) and MsCl (0.11 mL, 1.42 mmol) and the reaction mixture was stirred at rt for 2 h. The reaction mixture was concentrated in vacuo and the crude material was purified by silica gel chromatography, eluting with a gradient of 0-60% EtOAc in heptane, to provide Intermediate 3-061.4. MS (ESI) m/z: 553.0 (M+H)⁺.

Step 5: 4-Methyl-8-(6-((2R,3R)-2-methyl-3-(piperazin-1-yl)azetidin-1-yl)-2-(trifluoromethyl)pyrimidin-4-yl)-1-oxa-8-azaspiro[4.5]dec-3-ene (TFA salt), Intermediate 3-061.5. To a solution of Intermediate 3-061.4 (179 mg, 0.32 mmol) in DCM (2 mL) was added TFA (0.5 mL, 0.32 mmol) and the reaction mixture was stirred for 30 min at rt. The solvent was removed to provide Intermediate 3-061.5, as a TFA salt which was taken to next step. MS (ESI) m/a: 453.2 (M+H)⁺.

Step 6. The crude reaction mixture from Step 5 was redissolved in DCM (2 mL) and DIPEA (0.56 mL, 3.24 mmol) followed by acryloyl chloride (0.2 M in DCM, 1.62 mL, 0.32 mmol) were added. The reaction mixture was stirred at rt for 30 min and quenched by the addition of sat. aq. NaHCO₃ and extracted with DCM. The organic extracts were washed with sat. aq. NH₄Cl, dried over Na₂SO₄, and concentrated in vacuo. The crude material was purified by silica gel chromatography, eluting with a gradient of 0-40% EtOAc/EtOH (3:1) in heptane. The material was further purified by reverse-phase preparative HPLC using 0.1% TFA in ACN/H₂O as eluent, with a gradient 10-90% over 15 min. The fractions containing product were free based by washing with sat. aq. Na₂CO₃ and extracted with EtOAc. The organic extracts were washed with brine, dried over Na₂SO₄, filtered, and concentrated in vacuo to provide a crude mixture of Compound 3-061 and 1-(4-((2R,3R)-2-methyl-1-(6-(4-methylene-1-oxa-8-azaspiro[4.5]decan-8-yl)-2-(trifluoromethyl)pyrimidin-4-yl)azetidin-3-yl)piperazin-1-yl)prop-2-en-1-one.

Step 7: 1-(4-((2R,3R)-2-Methyl-1-(6-(4-methyl-1-oxa-8-azaspiro[4.5]dec-3-en-8-yl)-2-(trifluoromethyl)pyrimidin-4-yl)azetidin-3-yl)piperazin-1-yl) prop-2-en-1-one, Compound 3-061. The above mixture was purified via SFC using an OJ, 21×250 mm, 5 μm column with a mobile phase of 25% MeOH and a flowrate of 80 mL/min. The first eluting peak was identified as Compound 3-061. MS (ESI) m/z: 507.2 (M+H)⁺. ¹H NMR (400 MHz, CDCl₃) δ 6.50-6.63 (m, 1H), 6.30 (dd, J=16.8, 1.9 Hz, 1H), 5.71 (dd, J=10.6, 1.9 Hz, 1H), 5.46-5.55 (m, 1H), 5.17-5.27 (m, 1H), 4.45-4.61 (m, 3H), 4.19-4.33 (m, 2H), 3.89-4.04 (m, 2H), 3.55-3.80 (m, 4H), 3.14-3.35 (m, 3H), 2.29-2.43 (m, 4H), 1.69-1.81 (m, 2H), 1.66 (q, J=1.9 Hz, 3H), 1.52 (br s, 2H), 1.48 (d, J=6.4 Hz, 3H), ¹⁹F NMR (377 MHz, CDCl₃) δ −71.77 (s, 3F).

### Example 3-062-1:4-(3-(4-Acryloylpiperazin-1-yl)azetidin-1-yl)-6-((6S)-6-(hydroxymethyl)-1-oxa-8-azaspiro[4.5]decan-8-yl)-2-(trifluoromethyl)nicotinonitrile

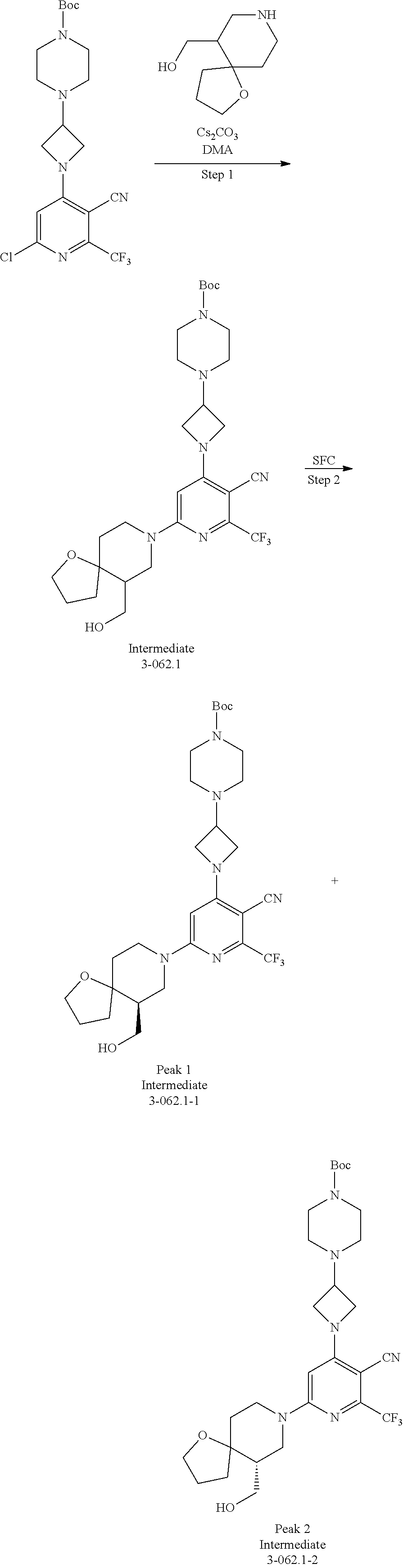 [SMILES: CC(C)(C)OC(=O)N1CCN(C2CN(c3cc(Cl)nc(C(F)(F)F)c3C#N)C2)CC1 ; OCC1CNCCC12CCCO2 ; CC(C)(C)OC(=O)N1CCN(C2CN(c3cc(N4CCC5(CCCO5)C(CO)C4)nc(C(F)(F)F)c3C#N)C2)CC1 ; "SFC" ; CC(C)(C)OC(=O)N1CCN(C2CN(c3cc(N4CCC5(CCCO5)[C@H](CO)C4)nc(C(F)(F)F)c3C#N)C2)CC1 ; CC(C)(C)OC(=O)N1CCN(C2CN(c3cc(N4CCC5(CCCO5)[C@@H](CO)C4)nc(C(F)(F)F)c3C#N)C2)CC1]

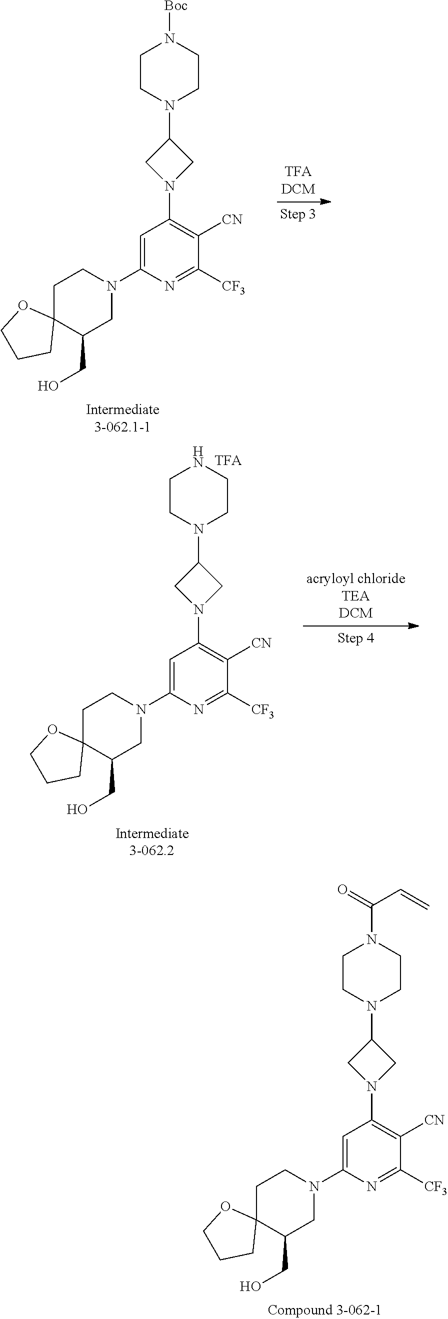 [SMILES: CC(C)(C)OC(=O)N1CCN(C2CN(c3cc(N4CCC5(CCCO5)[C@H](CO)C4)nc(C(F)(F)F)c3C#N)C2)CC1 ; N#Cc1c(N2CC(N3CCNCC3)C2)cc(N2CCC3(CCCO3)[C@H](CO)C2)nc1C(F)(F)F ; C=CC(=O)N1CCN(C2CN(c3cc(N4CCC5(CCCO5)[C@H](CO)C4)nc(C(F)(F)F)c3C#N)C2)CC1]

Step 1: tert-Butyl 4-(1-(3-cyano-6-(6-(hydroxymethyl)-1-oxa-8-azaspiro[4.5]decan8-yl)-2-(trifluoromethyl)pyridin-4-yl)azetidin-3-yl)piperazine-1-carboxylate, Intermediate 3-062.1. To a degassed solution of tert-butyl 4-(1-(6-chloro-3-cyano-2-(trifluoromethyl)pyridin-4-yl)azetidin-3-yl)piperazine 1-carboxylate (391 mg, 0.88 mmol) in DMA (5 mL), was added (1-oxa-8-azaspiro[4.5]decan-6-yl)methanol (150 mg, 0.88 mmol) and Cs₂CO₃ (856 mg, 2.63 mmol). The reaction mixture was heated at 90° C. for 16 h, diluted with H₂O (500 mL), and extracted with DCM (2×50 mL). The combined organic extracts were washed with H₂O (50 mL), brine (50 mL), dried over Na₂SO₄, filtered, concentrated and purified by silica gel chromatography, eluting with 5-10% MeOH in DCM, to afford Intermediate 3-062.1. MS (ESI) m/z: 581.7 (M+H)⁺. ¹H NMR (DMSO-d₆, 400 MHz) δ 4.55 (t, J=4.9 Hz, 1H), 4.28 (s, 3H), 4.03 (t, J=7.2 Hz, 3H), 3.86-3.67 (m, 2H), 3.65-3.58 (m, 1H), 3.50 (dd, J=12.7, 6.2 Hz, 2H), 3.35 (d, J=3.5H), 3.27 (s, 2H), 3.22-3.15 (m, 1H), 2.30 (t, J=5.0 Hz, 4H), 1.98 (d, J=12.8 Hz, 2H), 1.92-1.72 (m, 2H), 1.69-1.58 (m, 2H), 1.54-1.43 (m, 1H), 1.40 (s, 9H).

Step 2:1-tert-Butyl 4-(1-(3-cyano-6-((6S)-6-(hydroxymethyl)-1-oxa-8-azaspiro[4.5]decan-8-yl)-2-(trifluoromethyl)pyridin-4-yl)azetidin-3-yl)piperazine-1-carboxylate, Intermediate 3-062.1~1; tert-butyl 4-(1-(3-cyano-6-((6R)-6-(hydroxymethyl)-1-oxa-8-azaspiro[4.5]decan-8-yl)-2-(trifluoromethyl)pyridin-4-yl)azetidin-3-yl)piperazine-1-carboxylate, Intermediate 3-062-1-2. The stereoisomers were separated by SFC using a Chiralpak IG (250×4.6 mm) column with a mobile phase of 25% MeOH and a flowrate of 75 mL/min. The first eluting peak was identified arbitrarily as Intermediate 3-062.1-1. MS (ESI) m/z: 581.7 (M+H)⁺. ¹H NMR (DMSO-d₆, 400 MHz) δ 5.78-5.71 (m, 1H), 4.55 (t, J=4.8 Hz, 1H), 4.28 (s, 3H), 4.02 (dd, J=10.3, 3.8 Hz, 3H), 3.86-3.75 (m, 1H), 3.78-3.67 (m, 2H), 3.65-3.56 (m, 1H), 3.50 (dt, J=8.3, 5.3 Hz, 2H), 3.34 (d, J=5.7 Hz, 5H), 3.28 (s, 3H), 3.26 (d, J=5.2 Hz, OH), 3.24-3.14 (m, 1H), 2.30 (t, J=5.0 Hz, 5H), 1.95-1.72 (m, 3H), 1.67 (s, 2H), 1.71-1.58 (m, 1H), 1.54-1.43 (m, 1H), 1.40 (s, 9H).

The second eluting peak was identified arbitrarily as Intermediate 3-062.1-2. MS (ESI) m/z: 581.7 (M+H)⁺. ¹H NMR (DMSO-d₆, 400 MHz) δ 5.78-5.71 (m, 2H), 4.55 (t, J=4.9 Hz, 1H), 4.27 (s, 3H), 4.02 (d, J=8.6 Hz, 3H), 3.86-3.73 (m, 2H), 3.71 (s, 1H), 3.65-3.55 (m, 1H), 3.50 (dd, J=12.8, 6.4 Hz, 2H), 3.34 (d, J=5.5 Hz, 5H), 3.29-3.14 (m, 2H), 2.30 (t, J=5.0 Hz, 6H), 2.05-1.97 (m, 1H), 1.92-1.72 (m, 3H), 1.67 (s, 2H), 1.71-1.58 (m, 1H), 1.54-1.43 (m, 1H), 1.47 (s, 1H), 1.40 (s, 9H).

Step 3: 6-((6S)-6-(Hydroxymethyl)-1-oxa-8-azaspiro[4.5]decan-8-yl)-4-(3-(piperazin-1-yl)azetidin-1-yl)-2-(trifluoromethyl)nicotinonitrile (TFA salt), Intermediate 3-062.2. Intermediate 3-0621-1 (120 mg, 0.21 mmol) in DCM (3 mL) was cooled to 0° C. and TFA (0.16 mL, 2.07 mmol) was added dropwise to the reaction mixture. The reaction mixture was stirred at rt for 2 h, then concentrated in vacuo to give Intermediate 3-062.2, as a TFA salt which was directly used in the next step. MS (ESI) m/z: 481.8 (M+H)⁺.

Step 4: 4-(3-(4-Acryloylpiperazin-1-yl)azetidin-1-yl)-6-(6S)-6-(hydroxymethyl)-1-oxa-8-azaspiro[4.5]decan-8-yl)-2-(trifluoromethyl)nicotinonitrile, Compound 3-062-1. To the crude mixture from Step 3 was added DCM (5 mL). The reaction mixture was cooled to 0° C. and TEA (0.11 mL, 0.82 mmol) and acryloyl chloride (19 mg, 0.20 mmol) were added. The reaction mixture was stirred for 5 min, diluted with H₂O, and extracted with DCM (3×10 mL). The organic extracts were dried over Na₂SO₄ and concentrated in vacuo. The crude material was purified by prep HPLC eluting with a gradient of 0% to 70% (0.1% NH₃) H₂O in ACN to provide Compound 3-062-1. MS (ESI) m/z: 535.7 (M+H)⁺. ¹H NMR (DMSO-d₆, 400 MHz) δ 6.81 (dd, J=16.7, 10.5 Hz, 1H), 6.11 (dd, J=16.7, 2.4 Hz, 1H), 5.75 (d, J=9.2 Hz, 1H), 5.68 (dd, J=10.4, 2.5 Hz, 1H), 4.58-4.52 (m, 1H), 4.29 (t, J=7.9 Hz, 2H), 4.05 (t, J=7.2 Hz, 2H), 3.86-3.65 (m, 2H), 3.65-3.57 (m, 1H), 3.57 (s, 5H), 3.55-3.45 (m, 1H), 3.31 (s, 1H), 3.29-3.13 (m, 1H), 2.35 (s, 4H), 1.83 (m, 2H), 1.65 (m, 1H), 1.57-1.40 (m, 1H), 1.47 (s, 1H).

The example below was prepared in a manner similar to that described above using a similar spirocyclic intermediate.

TABLE 2-4

| | | | MS (ESI) |
|---|---|---|---|
| Compound | | | m/z: |
| No. | Chemical Structure | Name | (M + H)⁺ |
| 3-062-2 | 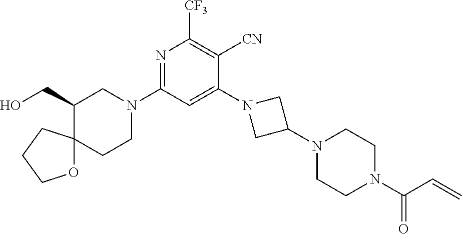 [SMILES: C=CC(=O)N1CCN(C2CN(c3cc(N4CCC5(CCCO5)[C@@H](CO)C4)nc(C(F)(F)F)c3C#N)C2)CC1] | 4-(3-(4-acryloylpiperazin-1- yl)azetidin-1-yl)-6-((6R)-6- (hydroxymethyl)-1-oxa-8- azaspiro[4.5]decan-8-yl)-2- (trifluoromethyl)nicotinonitrile | 535.7 |

### Example 3-063:1-(4-(1-(6-(2,3-Dihydrospiro[indene-1,4′-piperidin]-1′-yl)-2-(trifluoromethyl)pyrimidin-4-yl)azetidin-3-yl)piperazin-1-yl) prop-2-en-1-one

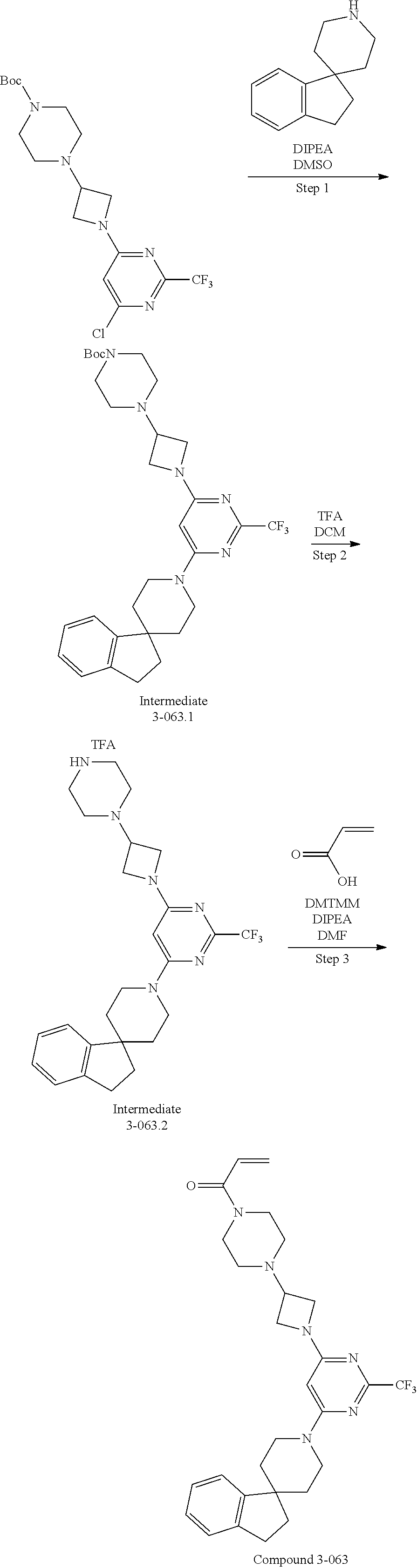

Step 1: tert-Butyl 4-(1-(6-(2,3-dihydrospiro[indene-1,4′-piperidin]-1′-yl)-2-(trifluoromethyl)pyrimidin-4-yl)azetidin-3-yl)piperazine-1-carboxylate, Intermediate 3-063.1. To a mixture of 2,3-dihydrospiro[indene-1,4′-piperidine] (1.2 μmol) and ter-butyl 4-(1-(6-chloro-2-(trifluoromethyl)pyrimidin-4-yl)azetidin-3-yl)piperazine-1-carboxylate (1.2 μmol) in dry DMSO was added DIPEA (2.6 μL, 15 μmol). The solution was shaken at 80° C. for 16 h, then at 100° C. for 4 h. The reaction mixture was concentrated to provide crude material containing Intermediate 3-063.1, which was used directly in the next step.

Step 2: 1′-(6-(3-(Piperazin-1-yl)azetidin-1-yl)-2-(trifluoromethyl)pyrimidin-4-yl)-2,3-dihydrospiro[indene-1,4′-piperidine] (TFA salt), Intermediate 3-063.2. TFA (50 μL 30% in DCM) was added to the concentrated product of Step 1, and shaken for 2 h at rt. The reaction mixture was concentrated to provide crude material containing Intermediate 3-063.2, as a TFA salt, which was used directly in the next step.

Step 3:1-(4-(1-(6-(2,3-Dihydrospiro[indene-1,4′-piperidin]-1′-yl)-2-(trifluoromethyl)pyrimidin-4-yl)azetidin-3-yl)piperazin-1-yl) prop-2-en-1-one. Compound 3-063. To the crude mixture from Step 2 was added a 100 mM solution of acrylic acid (24 μL) in dry DMF, 12 μL of a 200 mM solution DMTMM in dry DMF, and 12 μL of 1 M DIPEA in DMF. The solution was shaken at rt for 16 h. The reaction mixture was then concentrated to provide Compound 3-063. MS (ESI) m/z: 527.3 (M+H)⁺.

The below example was prepared in a manner similar to that described above.

TABLE 2-5

| | | | MS (ESI) |
|---|---|---|---|
| Compound | | | m/z: |
| No. | Chemical Structure | Name | (M + H)⁺ |
| 3-064 | 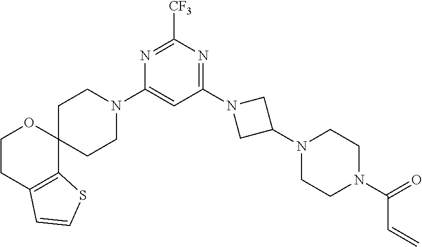 [SMILES: C=CC(=O)N1CCN(C2CN(c3cc(N4CCC5(CC4)OCCc4ccsc45)nc(C(F)(F)F)n3)C2)CC1] | 1-(4-(1-(6-(4′,5′- dihydrospiro[piperidine-4,7′- thieno[2,3-c]pyran]-1-yl)-2- (trifluoromethyl)pyrimidin-4- yl)azetidin-3-yl)piperazin-1- yl)prop-2-en-1-one | 549.2 |

### Example 3-065:4-(3-(4-Acryloylpiperazin-1-yl)azetidin-1-yl)-6-(4-chloro-2-methyl-1-oxa-8-azaspiro[4.5]dec-3-en-8-yl)-2-(trifluoromethyl)nicotinonitrile

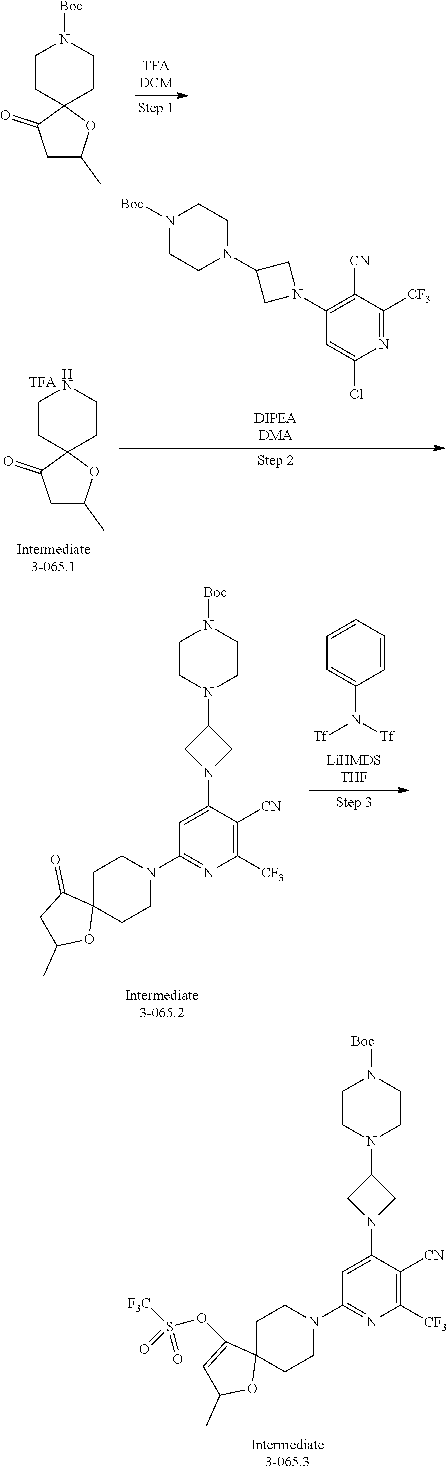

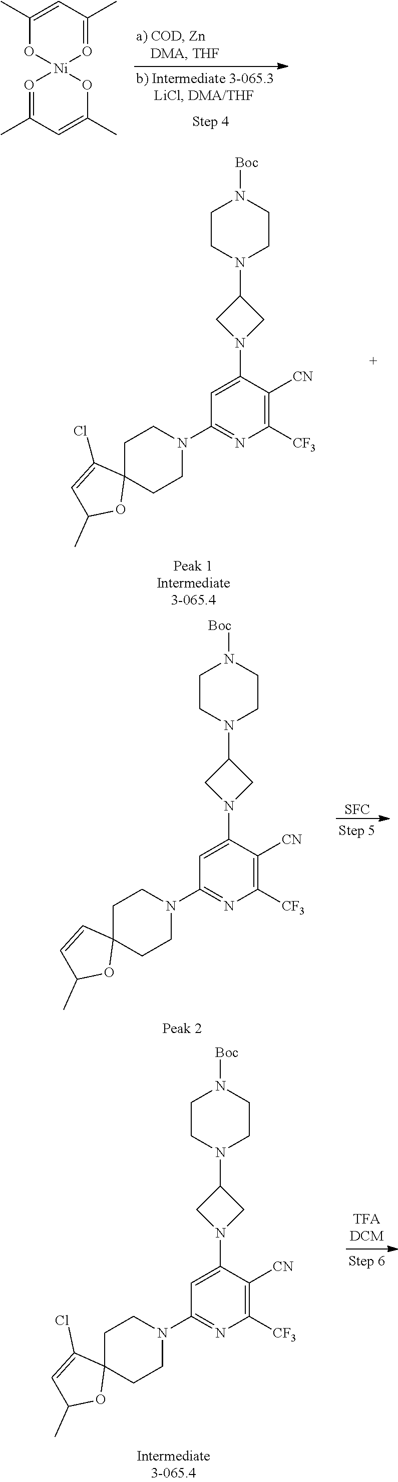

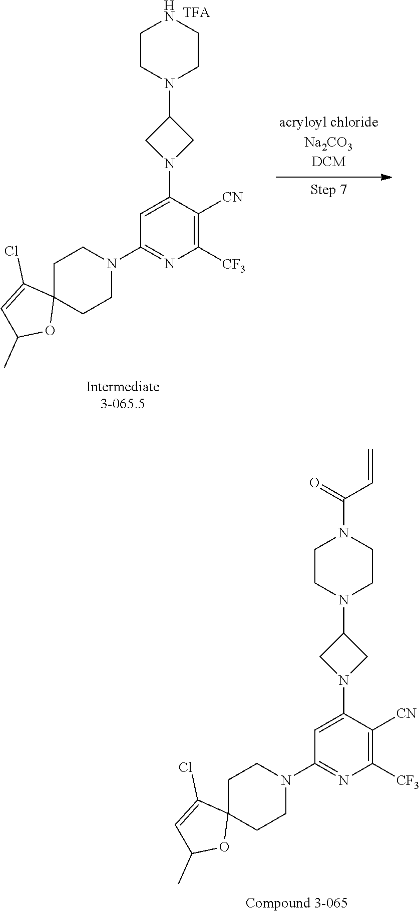 [SMILES: CC1C=C(Cl)C2(CCN(c3cc(N4CC(N5CCNCC5)C4)c(C#N)c(C(F)(F)F)n3)CC2)O1 ; C=CC(=O)N1CCN(C2CN(c3cc(N4CCC5(CC4)OC(C)C=C5Cl)nc(C(F)(F)F)c3C#N)C2)CC1]

Step 1: 2-Methyl-1-oxa-8-azaspiro[4.5]decan-4-one (TFA salt), Intermediate 3-065.1. To a solution of tert-butyl 2-methyl-4-oxo-1-oxa-8-azaspiro[4.5]decane-8-carboxylate (301 mg, 1.12 mmol) in DCM (5.6 mL) was added TFA (1.7 mL, 22.4 mmol) at rt. The reaction was stirred at rt for 60 min. The reaction was concentrated to provide Intermediate 3-065.1, as a TFA salt, which was used directly in the next step. MS (ESI) m/z: 170.2 (M+H)⁺.

Step 2: tert-Butyl 4-(1-(3-cyano-6-(2-methyl-4-oxo-1-oxa-8-azaspiro[4.5]decan-8-yl)-2-(trifluoromethyl)pyridin-4-yl)azetidin-3-yl)piperazine-1-carboxylate, Intermediate 3-065.2. To the concentrated reaction mixture of Step 1 was added DIPEA (2.0 mL, 11.2 mmol) in DMA (5.6 mL) at rt. The reaction mixture was stirred for 10 min at rt, and tert-butyl 4-(1-(6-chloro-3-cyano-2-(trifluoromethyl)pyridin-4-yl)azetidin-3-yl)piperazine-1-carboxylate (448 mg, 1.01 mmol) was added. The reaction mixture was heated at 86° C. for 24 h. The reaction was quenched by sat. aq. NH₄Cl (10 mL) and EtOAc (10 mL). The organic phase was washed with H₂O (8 mL) and sat. aq. NH₄Cl (2×), the combined aqueous layers were washed with EtOAc (8 mL). The organic phases were combined, washed with brine, dried over Na₂SO₄, and concentrated. The crude material was purified by silica gel chromatography, eluting with a gradient of 10-90% EtOAc in heptane, to provide Intermediate 3-065.2. MS (ESI) m/z: 579.2 (M+H)⁺.

Step 3: tert-Butyl 4-(1-(3-cyano-6-(2-methyl-4-(((trifluoromethyl)sulfonyl)oxy)-1-oxa-8-azaspiro[4.5]dec-3-en-8-yl)-2-(trifluoromethyl)pyridin-4-yl)azetidin-3-yl)piperazine-1-carboxylate, Intermediate 3-065.3. N-Phenyl bis-trifluoromethane sulfonimide (511 mg, 1.43 mmol) and tert-butyl 4-(1-(3-cyano-6-(2-methyl-4-oxo-1-oxa-8-azaspiro[4.5]decan-8-yl)-2-(trifluoromethyl)pyridin-4-yl)azetidin-3-yl)piperazine-1-carboxylate (690 mg, 1.19 mmol) were combined, and the reaction vessel was evacuated and backfilled with N₂. To the reaction mixture was added THF (4 mL), cooled to 0° C., added LiHMDS (1.0 M in THF, 1.43 mL, 1.43 mmol), and stirred at rt for 16 h. The reaction mixture was quenched by the addition of sat. aq. NH₄Cl and extracted with EtOAc. The organic extracts were washed with brine, dried over Na₂SO₄, filtered, and concentrated in vacuo. The crude material was purified by silica gel chromatography, eluting with a gradient of 0-40% EtOAc in heptane, to provide Intermediate 3-065.3. MS (ESI) m/z: 711.2 (M+H)⁺.

Step 4: tert-Butyl 4-(1-(6-(4-chloro-2-methyl-1-oxa-8-azaspiro[4.5]dec-3-en-8-yl)-3-cyano-2-(trifluoromethyl)pyridin-4-yl)azetidin-3-yl)piperazine-1-carboxylate, Intermediate 3-065.4, 2,4-Pentanedione nickel(II) (11.46 mg, 0.045 mmol), 1,5-cyclooctadiene (9.7 mg, 0.089 mmol), and zinc dust (5.8 mg, 0.089 mmol) in DMA (1 mL) and THF (2.2 mL) were stirred at rt for 30 min and sonicated for 3-5 min. Intermediate 3-065.3 (317 mg, 0.446 mmol) and LiCl (37.8 mg, 0,892 mmol) in DMA (2 mL) and THF (2.2 mL) were combined and the catalyst mixture was added. The resulting mixture was stirred at rt for 24 h. The crude reaction was concentrated and the crude material was purified by silica gel chromatography, eluting with a gradient of 10-80% EtOAc in heptane, to provide tert-butyl 4-(1-(3-cyano-6-(2-methyl-1-oxa-8-azaspiro[4.5]dec-3-en-8-yl)-2-(trifluoromethyl)pyridin-4-yl)azetidin3-yl)piperazine-1-carboxylate (MS (ESI) m/z: 585.0 (M+23)⁺) and Intermediate 3-065.4.

Step 5. The products from Step 4 were separated by SFC (2-EP 30×150 mm column with MeOH as co-solvent with a 5-15% gradient and 100 mL/min flow rate), to provide Intermediate 3-065.4. MS (ESI) m/z: 597.2 (M+H)⁺

Step 6: 6-(4-Chloro-2-methyl-1-oxa-8-azaspiro[4.5]dec-3-en-8-yl)-4-(3-(piperazin-1-yl)azetidin-1-yl)-2-(trifluoromethyl)nicotinonitrile (TFA salt), Intermediate 3-065.5. Intermediate 3-065.4 (136 mg, 0.23 mmol), TFA (339 μL, 4.56 mmol), and DCM (2.5 mL) were stirred at rt for 1 h. The reaction was concentrated to provide Intermediate 3-065.5, as a TFA salt, which was used directly in the next step. MS (ESI) m/z: 497.2 (M+H)⁺.

Step 7:4-(3-(4-Acryloylpiperazin-1-yl)azetidin-1-yl)-6-(4-chloro-2-methyl-1-oxa-8-azaspiro[4.5]dec-3-en-8-yl)-2-(trifluoromethyl)nicotinonitrile, Compound 3-065. To the crude mixture of Step 6 was added Na₂CO₃ (3.62 g, 3.42 mmol) followed by acryloyl chloride (0.2 M in DCM, 1.4 mL, 0.23 mmol) at 0° C. and stirred for 15 min. The reaction was extracted with DCM (2×3 mL). The organic extracts were dried over Na₂SO₄ and concentrated. The crude material was purified by pre TLC to give Compound 3-065. MS (ESI) m/z: 550.9 (M+H)⁺. ¹H NMR (400 MHz, CDCl₃) δ pm 6.57 (dd, J=16.7, 10.7 Hz, 1H), 6.31 (dd, J=16.7, 1.9 Hz, 1H), 5.80 (d, J=1.7 Hz, 1H), 5.73 (dd, J=10.5, 1.9 Hz, 1H), 5.41 (s, 1H), 4.93 (qd, J=6.4, 1.5 Hz, 1H), 4.26-4.43 (m, 4H), 4.13 (br s, 2H), 3.54-3.84 (m, 4H), 3.22-3.39 (m, 3H), 2.45 (br s, 4H), 2.01 (td, J=13.1, 4.9 Hz, 1H), 1.89 (td, J=13.1, 4.9 Hz, 1H), 1.52-1.66 (m, 2H), 1.33 (d, J=6.3 Hz, 3H). ¹⁹F NMR (376 MHz, CDCl₃) δ −66.73 (s, 3F).

The below example was prepared in a manner similar to that described above

TABLE 2-6

| | | | MS (ESI) |
|---|---|---|---|
| Compound | | | m/z: |
| No. | Chemical Structure | Name | (M + H)⁺ |
| 3-066 | 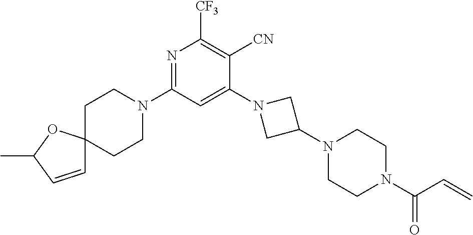 [SMILES: C=CC(=O)N1CCN(C2CN(c3cc(N4CCC5(C=CC(C)O5)CC4)nc(C(F)(F)F)c3C#N)C2)CC1] | 4-(3-(4-acryloylpiperazin-1- yl)azetidin-1-yl)-6-(2-methyl-1- oxa-8-azaspiro[4.5]dec-3-en-8- yl)-2- (trifluoromethyl)nicotinonitrile | 517.2 |

### Examples 3-067-1 and 3-067-2: (R)-4-(3-(4-Acryloylpiperazin-1-yl)azetidin-1-yl)-6-(4-chloro-2-methyl-1-oxa-8-azaspiro[4.5]dec-3-en-8-yl)-2-(trifluoromethyl)nicotinonitrile; (S)-4-(3-(4-Acryloylpiperazin-1-yl)azetidin-1-yl)-6-(4-chloro-2-methyl-1-oxa-8-azaspiro[4.5|dec-3-en-8-yl)-2-(trifluoromethyl)nicotinonitrile

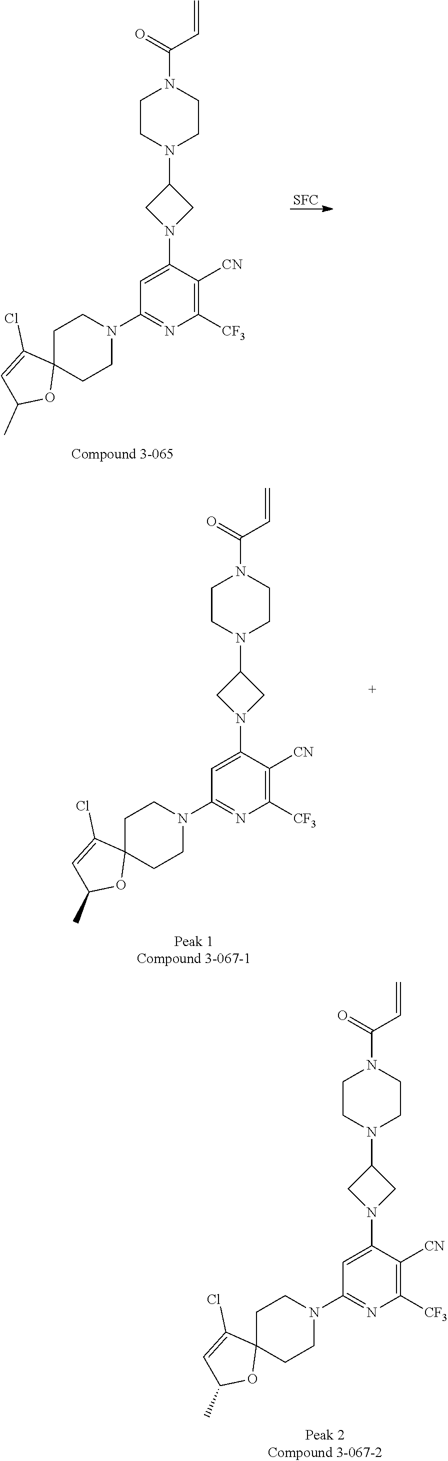 [SMILES: C=CC(=O)N1CCN(C2CN(c3cc(N4CCC5(CC4)OC(C)C=C5Cl)nc(C(F)(F)F)c3C#N)C2)CC1 ; "SFC" ; C=CC(=O)N1CCN(C2CN(c3cc(N4CCC5(CC4)O[C@@H](C)C=C5Cl)nc(C(F)(F)F)c3C#N)C2)CC1 ; C=CC(=O)N1CCN(C2CN(c3cc(N4CCC5(CC4)O[C@H](C)C=C5Cl)nc(C(F)(F)F)c3C#N)C2)CC1]

Compound 3-065 was purified via SFC using a ChiralPak AZ, 2×25 cm 5 μm column with a mobile phase of 50% MeOH and a flowrate of 80 mL/min. The first eluting isomer was identified and the stereochemistry arbitrarily assigned as (R)-4-(3-(4-acryloylpiperazin-1-yl)azetidin-1-yl)-6-(4-chloro-2-methyl-1-oxa-8-azaspiro[4.5]dec-3-en-8-yl)-2-(trifluoromethyl)nicotinonitrile (Compound 3-067-1). MS (ESI) m/z: 551.2 (M+H)⁺. ¹H NMR (600 MHz, DMSO-d₆) δ 6.81 (dd, J=16.7, 10.4 Hz, 1H), 6.08-6.14 (m, 2H), 5.79 (s, 1H), 5.68 (dd, J=10.4, 2.3 Hz, 1H), 4.89 (qd, J=6.4, 1.3 Hz, 1H), 4.23-4.42 (m, 4H), 4.07 (br d, J=3.1 Hz, 2H), 3.51-3.64 (m, 3H), 3.25-3.31 (m, 1H), 3.14 (br t, J=13.1 Hz, 2H), 2.34 (br s, 3H), 1.83 (td, J=13.2, 4.8 Hz, 1H), 1.70 (td, J=13.2, 4.8 Hz, 1H), 1.47-1.58 (m, 2H), 1.26 (d, J=6.3 Hz, 3H).

The second eluting isomer was arbitrarily assigned as (S)-4-(3-(4-acryloylpiperazin-1-yl)azetidin-1-yl)-6-(4-chloro-2-methyl-1-oxa-8-azaspiro[4.5]dec-3-en-8-yl)-2-(trifluoromethyl)nicotinonitrile (Compound 3-067-2). MS (ESI) m/z: 551.2 (M+H)⁺. ¹H NMR (600 MHz, DMSO-d₆) δ 6.81 (dd, J=16.7, 10.5 Hz, 1H), 6.07-6.14 (m, 2H), 5.79 (s, 1H), 5.68 (dd, J=10.4, 2.3 Hz, 1H), 4.89 (qd, J=6.3, 1.2 Hz, 1H), 4.25-4.41 (m, 4H), 4.07 (br s, 2H), 3.52-3.63 (m, 3H), 3.25-3.31 (m, 1H), 3.07-3.20 (m, 2H), 2.27-2.40 (m, 3H), 1.83 (td, J=13.2, 4.8 Hz, 1H), 1.70 (td. J=13.2, 4.8 Hz, 1H), 1.46-1.59 (m, 2H), 1.26 (d, J=6.3 Hz, 3H).

Examples in the table below were purified in a manner similar to that described above.

TABLE 2-7

| | | | Racemic SM \| |
|---|---|---|---|
| Compound | | | separation |
| No | Chemical Structure | Name | condition |
| 3-068-1 | 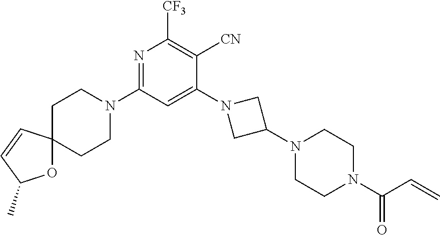 [SMILES: C=CC(=O)N1CCN(C2CN(c3cc(N4CCC5(C=C[C@@H](C)O5)CC4)nc(C(F)(F)F)c3C#N)C2)CC1] | (R)-4-(3-(4-acryloylpiperazin- 1-yl)azetidin-1-yl)-6-(2- methyl-1-oxa-8- azaspiro[4.5]dec-3-en-8-yl)-2- (trifluoromethyl)nicotinonitrile | SFC using a Lux Cellulose-2, 2 × 25 cm 5 μm column with a mobile phase of 30% 1:1 ACN:MeOH using a flowrate of 80 mL/min. 517.2 (M + H)⁺ |
| 3-068-2 | 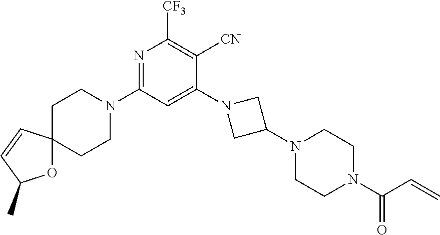 [SMILES: C=CC(=O)N1CCN(C2CN(c3cc(N4CCC5(C=C[C@H](C)O5)CC4)nc(C(F)(F)F)c3C#N)C2)CC1] | (S)-4-(3-(4-acryloylpiperazin- 1-yl)azetidin-1-yl)-6-(2- methyl-1-oxa-8- azaspiro[4.5]dec-3-en-8-yl)-2- (trifluoromethyl)nicotinonitrile | SFC using a Lux Cellulose-2, 2 × 25 cm 5 μm column with a mobile phase of 30% 1:1 ACN:MeOH using a flowrate of 80 mL/min. 517.2 (M + H)⁺ |

### Example 3-069:1-(4-(1-(2-(Difluoromethyl)-6-(6′,7′-dihydrospiro[piperidine-4,4′-pyrano[4,3-d]thiazol]-1-yl)-3-methylpyridin-4-yl)azetidin-3-yl)piperazin-1-yl) prop-2-en-1-one

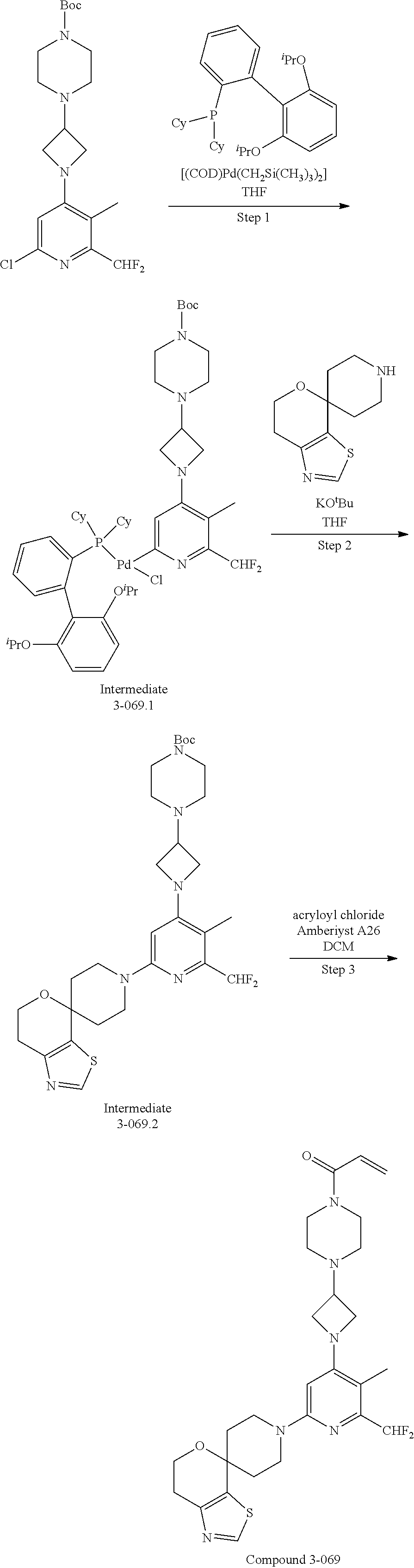

Step 1:(4-(3-(4-(tert-Butoxycarbonyl)piperazin-1-yl)azetidin-1-yl)-6-(difluoromethyl)-5-methylpyridin-2-yl)(dicyclohexyl(2′,6′-diisopropoxy-[1,1′-biphenyl]-2-yl)-15-phosphaneyl) palladium (III) chloride, Intermediate 3-069.1. tert-Butyl 4-(1-(6-chloro-2-(difluoromethyl)-3-methylpyridin-4-yl)azetidin-3-yl)piperazine-1-carboxylate (116 mg, 0.28 mmol), dicyclohexyl(2′,6′-diisopropoxy-[1,1′-biphenyl]-2-yl)phosphane (144 mg, 0.31 mmol), and (1,5-cyclooctadiene)bis(trimethylsilylmethyl) palladium(II) (118 mg, 0.30 mmol) were combined and degassed by 3 vacuum/argon cycles. THF (2 mL) was added and degassed by 3 additional vacuum/argon cycles. The reaction mixture was shaken at rt for 16 h followed by filtration of the reaction mixture through a 0.2 μm filter. The solvent was removed and the residue triturated with cold heptane and the supernatant removed with a syringe. The crude powder was then washed with cold heptane and the supernatant removed with a syringe to provide Intermediate 3-069.1, that was taken to next step.

Step 2: tert-Butyl 4-(1-(2-(difluoromethyl)-6-(6′,7′-dihydrospiro[piperidine-4,4′-pyrano[4,3-d]thiazol]-1-yl)-3-methylpyridin-4-yl)azetidin-3-yl)piperazine-1-carboxylate, Intermediate 3-069.2. A 400 nM solution of 6′,7′-dihydrospiro[piperidine-4,4′-pyrano[4,3-d]thiazole] in DMSO was prepared, then KOtBu (1 M in THF, 2.4 μL) was added followed by 12 μL of a 100 mM solution of the crude material from Step 1. The reaction was mixed at rt for 16 h. The reaction mixture was concentrated and reconstituted in 100 μL DCM. The supernatant was filtered through celite. This process was repeated with 2×50 μL DCM. The celite was washed with 2×100 μL DCM and the filtrate was air dried. The material was purified by chromatography, eluting with a gradient of 2-100% (0.1% HCOOH) MeOH in (0.1% HCOOH) H₂O in with a flow rate 5-8 mL/min to provide Intermediate 3-069.2.

Step 3: 1-(4-(1-(2-(Difluoromethyl)-6-(6′,7′-dihydrospiro[piperidine-4,4′-pyrano[4,3-d]thiazol]-1-yl)-3-methylpyridin-4-yl)azetidin-3-yl)piperazin-1-yl) prop-2-en-1-one, Compound 3-069. Intermediate 3-069.2 from Step 2 was dissolved in 30 μL of DCM, followed by 24 μL of a 100 mM solution of acryloyl chloride in DCM. The reaction was stirred at rt for 10 min. Amberlyst A26 was added to the reaction, then the reaction stirred for 10 min at rt. The Amberlyst A26 was removed and the reaction mixture washed with 40 μL of DCM×2. The crude reaction mixture was then concentrated to provide Compound 3-069. MS (ESI) m/z: 545.3 (M+H)⁺.

Examples in the table below were prepared in a manner similar to that described above.

TABLE 2-8

| | | | MS (ESI) |
|---|---|---|---|
| Compound | | | m/z: |
| No. | Chemical Structure | Name | (M + H)⁺ |
| 3-070 | 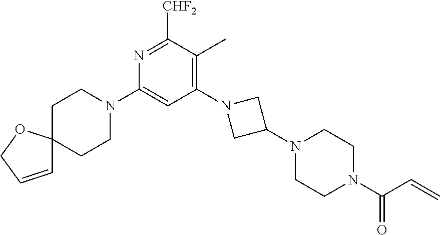 [SMILES: C=CC(=O)N1CCN(C2CN(c3cc(N4CCC5(C=CCO5)CC4)nc(C(F)F)c3C)C2)CC1] | 1-(4-(1-(2-(difluoromethyl)-3- methyl-6-(1-oxa-8- azaspiro[4.5]dec-3-en-8- yl)pyridin-4-yl)azetidin-3- yl)piperazin-1-yl)prop-2-en-1- one | 474.3 |
| 3-071 | 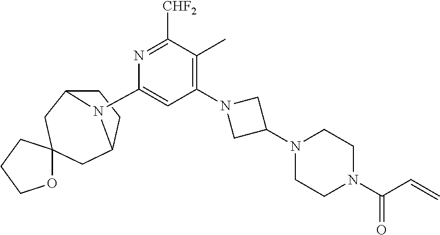 [SMILES: C=CC(=O)N1CCN(C2CN(c3cc(N4C5CCC4CC4(CCCO4)C5)nc(C(F)F)c3C)C2)CC1] | 1-(4-(1-(2-(difluoromethyl)-6- (dihydro-3′H-8- azaspiro[bicyclo[3.2.1]octane- 3,2′-furan]-8-yl)-3- methylpyridin-4-yl)azetidin-3- yl)piperazin-1-yl)prop-2-en-1- one | 502.3 |
| 3-072 | 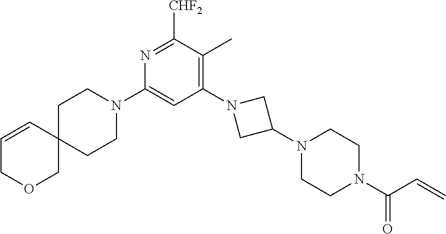 [SMILES: C=CC(=O)N1CCN(C2CN(c3cc(N4CCC5(C=CCOC5)CC4)nc(C(F)F)c3C)C2)CC1] | 1-(4-(1-(2-(difluoromethyl)-3- methyl-6-(2-oxa-9- azaspiro[5.5]undec-4-en-9- yl)pyridin-4-yl)azetidin-3- yl)piperazin-1-yl)prop-2-en-1- one | 488.3 |

### Example 3-073:1-(4-((2R,3R)-1-(6-(8-Azaspiro[4.5]dec-1-en-8-yl)-2-(trifluoromethyl)pyrimidin-4-yl)-2-methylazetidin-3-yl)piperazin-1-yl) prop-2-en-1-one

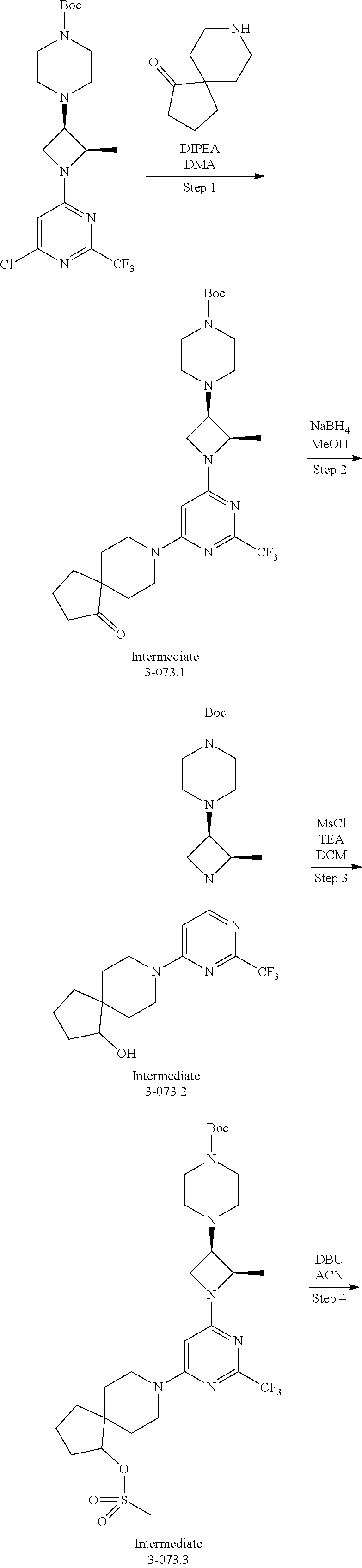 [SMILES: C[C@@H]1[C@H](N2CCN(C(=O)OC(C)(C)C)CC2)CN1c1cc(Cl)nc(C(F)(F)F)n1 ; O=C1CCCC12CCNCC2 ; C[C@@H]1[C@H](N2CCN(C(=O)OC(C)(C)C)CC2)CN1c1cc(N2CCC3(CCCC3=O)CC2)nc(C(F)(F)F)n1 ; C[C@@H]1[C@H](N2CCN(C(=O)OC(C)(C)C)CC2)CN1c1cc(N2CCC3(CCCC3O)CC2)nc(C(F)(F)F)n1 ; C[C@@H]1[C@H](N2CCN(C(=O)OC(C)(C)C)CC2)CN1c1cc(N2CCC3(CCCC3OS(C)(=O)=O)CC2)nc(C(F)(F)F)n1]

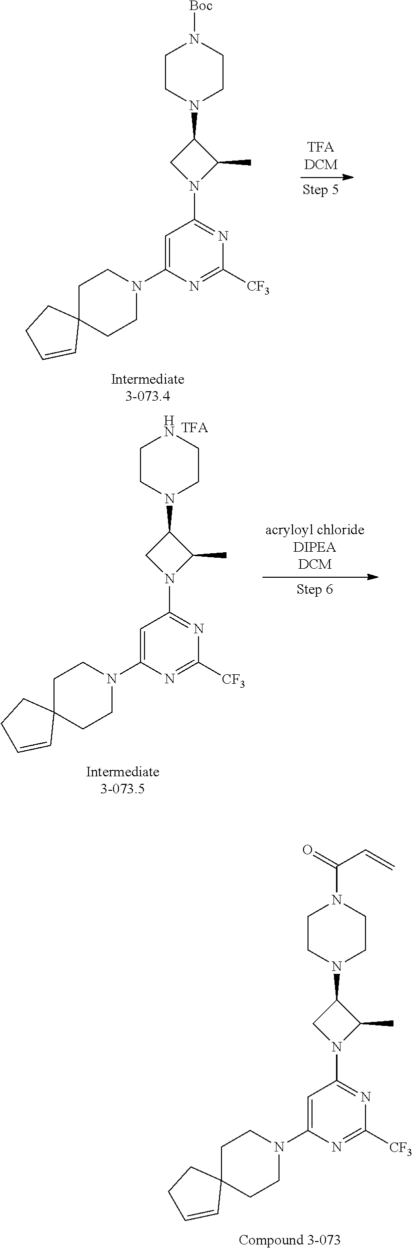 [SMILES: C[C@@H]1[C@H](N2CCN(C(=O)OC(C)(C)C)CC2)CN1c1cc(N2CCC3(C=CCC3)CC2)nc(C(F)(F)F)n1 ; C[C@@H]1[C@H](N2CCNCC2)CN1c1cc(N2CCC3(C=CCC3)CC2)nc(C(F)(F)F)n1 ; C=CC(=O)N1CCN([C@@H]2CN(c3cc(N4CCC5(C=CCC5)CC4)nc(C(F)(F)F)n3)[C@@H]2C)CC1]

Step 1: tert-Butyl 4-((2R,3R)-2-methyl-1-(6-(1-oxo-8-azaspiro[4.5]decan-8-yl)-2-(trifluoromethyl)pyrimidin-4-yl)azetidin-3-yl)piperazine-1-carboxylate, Intermediate 3-073.1. To a rbf was added tert-butyl 4-((2R,3R)-1-(6-chloro-2-(trifluoromethyl)pyrimidin-4-yl)-2-methylazetidin-3-yl)piperazine-1-carboxylate (500 mg, 1.15 mmol), 8-(2,2,2-trifluoroacetyl)-8-azaspiro[4.5]decan-1-one (287 mg, 1.15 mmol), DIPEA (1.0 mL, 5.74 mmol), and DMA (5 mL). The reaction mixture was heated at 100° C. for 16 h. Then, 1 equiv of DIPEA was added with continued heating for 3 h. The reaction mixture was concentrated and the crude material was purified by silica gel chromatography, eluting with a gradient of 0-40% EtOAc in petroleum ether, to provide Intermediate 3-073.1. MS (ESI) m/z: 553.2 (M+H)⁺.

Step 2: tert-Butyl 4-((2R,3R)-1-(6-(1-hydroxy-8-azaspiro[4.5]decan-8-yl)-2-(trifluoromethyl)pyrimidin-4-yl)-2-methylazetidin-3-yl)piperazine-1-carboxylate, Intermediate 3-073.2. To a 0° C. solation of Intermediate 3-073.1 (360 mg, 0.65 mmol) in MeOH (5 mL), was added NaBH₄ (37 mg, 0.98 mmol). After stirring for 4 h the reaction was concentrated, redissolved in DCM (50 mL), and washed with H₂O (75 mL). The organic extract was dried over Na₂SO₄, the solution was filtered, and concentrated in vacuo to give the crude Intermediate 3-073.2. MS (ESI) m/z: 555.3 (M+H)⁺.

Step 3: tert-Butyl 4-((2R,3R)-2-methyl-1-(6-(1-((methylsulfonyl)oxy)-8-azaspiro[4.5]decan-8-yl)-2-(trifluoromethyl)pyrimidin-4-yl)azetidin-3-yl)piperazine-1-carboxylate, Intermediate 3-073.3. Intermediate 3-073.2 (360 mg, 0.65 mmol) and TEA (0.136 mL, 0.974 mmol) in DCM (5 mL) were combined at 0° C. then MsCl (0.061 mL, 0.78 mmol) was added, and the reaction stirred for 4 h. The reaction mixture was diluted with H₂O (25 mL) and extracted with DCM (3×25 mL). The combined organic extracts were dried over Na₂SO₄ and concentrated in vacuo to give Intermediate 3-073.3. MS (ESI) m/z: 633.2 (M+H)⁺.

Step 4: tert-Butyl 4-((2R,3R)-1-(6-(8-azaspiro[4.5]dec-1-en-8-yl)-2-(trifluoromethyl)pyrimidin-4-yl)-2-methylazetidin-3-yl)piperazine-1-carboxylate, Intermediate 3-073.4. Intermediate 3-073.3 (36 mg, 0.57 mmol) and DBU (0.429 mL, 2.84 mmol) in ACN (5 mL) were stirred at rt for 5 h and then heated to 75° C. for 4 days. The reaction mixture was diluted with H₂O (30 mL) and extracted with EtOAc (3×40 mL). The combined organic extracts were dried over Na₂SO₄ filtered, and concentrated in vacuo. The crude material was purified by silica gel chromatography, eluting with a gradient of 0-30% EtOAc in petroleum ether to provide Intermediate 3-073.4. MS (ESI) m/z: 537.3 (M+H)⁺.

Step 5: 1-(4-((2R,3R)-1-(6-(8-Azaspiro[4.5]dec-1-en-8-yl)-2-(trifluoromethyl)pyrimidin-4-yl)-2-methylazetidin-3-yl)-114-piperazine (TFA salt), Intermediate 3-073.5. Intermediate 3-073.4 (180 mg, 0.34 mmol) and DCM (3 mL) were combined at 0° C. followed by TFA (0.26 mL, 3.35 mmol). The reaction mixture was stirred for 3 h, concentrated, and azeotroped with toluene twice to give Intermediate 3-073.5, as a TFA salt. MS (ESI) m/z: 437.3 (M+H)⁺.

Step 6:1-(4-((2R,3R)-1-(6-(8-Azaspiro[4.5]dec-1-en-8-yl)-2-(trifluoromethyl)pyrimidin-4-yl)-2-methylazetidin-3-yl)piperazin-1-yl) prop-2-en-1-one, Compound 3-073. To a solution of Intermediate 3-073.5 (180 mg, 0.34 mmol) in DCM (5 mL) at 0° C. was added DIPEA (1.69 mmol) and acryloyl chloride (0.027 mL, 0.34 mmol) and the reaction was stirred for 5 min. The reaction mixture was diluted with H₂O (25 mL) and extracted with DCM (2×20 mL). The combined organic extracts were dried over Na₂SO₄ and concentrated in vacuo to give the crude material which was purified by reverse phase HPLC to provide Compound 3-073. MS (ESI) m/s: 537.3 (M+H)⁺. ¹H NMR (400 MHz, DMSO-d₆) δ 6.80 (dd, J=16.7, 10.5 Hz, 1H), 6.11 (dd, J=16.7, 2.4 Hz, 1H), 5.79-5.65 (m, 3H), 5.58 (s, 1H), 4.48 (m, 1H), 4.03-3.74 (m, 4H), 3.57 (d, J=13.3 Hz, 4H), 3.40 (m, 2H), 3.24 (m, 1H), 2.42-2.18 (m, 6H), 1.74 (t, J=7.2 Hz, 2H), 1.51 (m, 2H), 1.40 (1, J=7.7 Hz, 5H).

### Example 3-074:1-(4-((2R,3R)-2-Methyl-1-(6-(4-methylene-1-oxa-8-azaspiro[4.5]decan8-yl)-2-(trifluoromethyl)pyrimidin-4-yl)azetidin-3-yl)piperazin-1-yl) prop-2-en-1-one

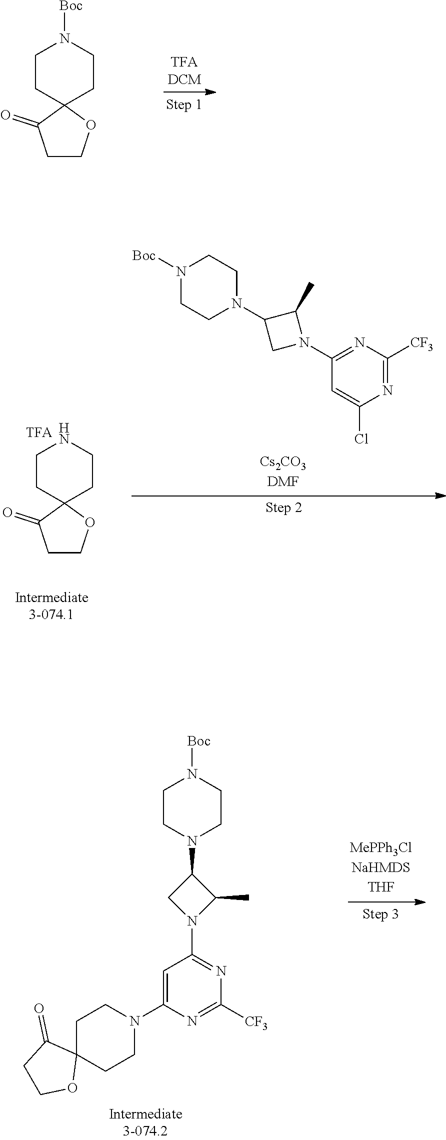 [SMILES: CC(C)(C)OC(=O)N1CCC2(CC1)OCCC2=O ; C[C@@H]1C(N2CCN(C(=O)OC(C)(C)C)CC2)CN1c1cc(Cl)nc(C(F)(F)F)n1 ; O=C1CCOC12CCNCC2 ; C[C@@H]1[C@H](N2CCN(C(=O)OC(C)(C)C)CC2)CN1c1cc(N2CCC3(CC2)OCCC3=O)nc(C(F)(F)F)n1]

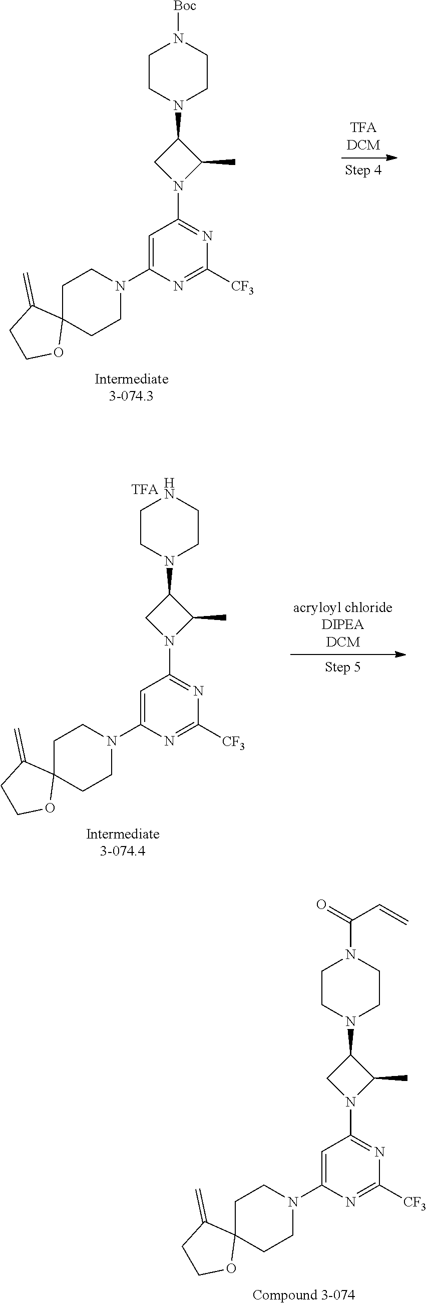 [SMILES: C=C1CCOC12CCN(c1cc(N3C[C@@H](N4CCN(C(=O)OC(C)(C)C)CC4)[C@H]3C)nc(C(F)(F)F)n1)CC2 ; C=C1CCOC12CCN(c1cc(N3C[C@@H](N4CCNCC4)[C@H]3C)nc(C(F)(F)F)n1)CC2 ; C=CC(=O)N1CCN([C@@H]2CN(c3cc(N4CCC5(CC4)OCCC5=C)nc(C(F)(F)F)n3)[C@@H]2C)CC1]

Step 1:1-Oxa-8-azaspiro[4.5]decan-4-one (TFA salt), Intermediate 3-074.1. To a solution of tert-butyl 4-oxo-1-oxa-8-azaspiro[4.5]decane-8-carboxylate (500 mg, 1.96 mmol) in DCM (3 mL) was added TFA (0.7 mL, 1.96 mmol) and the reaction mixture was stirred at rt for 30 min. The reaction mixture was concentrated in vacuo to provide Intermediate 3-074.1, as a TFA salt which was directly used without further purification. MS (ESI) m/z: 156.2 (M+H)⁺.

Step 2: tert-Butyl 4-((2R,3R)-2-methyl-1-(6-(4-oxo-1-oxa-8-azaspiro[4.5]decan-8-yl)-2-(trifluoromethyl)pyrimidin4-yl)azetidin-3-yl)piperazine-1-carboxylate, Intermediate 3-074.2. To the crude material from Step 1 was added Cs₂CO₃ (1.91 g, 5.88 mmol), tert-butyl 4-((2R,3R)-1-(6-chloro-2-(trifluoromethyl)pyrimidin-4-yl)-2-methylazetidin-3-yl)piperazine-1-carboxylate (854 mg, 1.96 mmol), and DMF (6 mL) and the reaction mixture was stirred at 100° C. for 6 h. The reaction mixture was washed with brine and extracted with EtOAc. The organic extracts were washed with brine, dried over Na₂SO₄, filtered, and concentrated in vacuo. The crude material was purified by silica gel chromatography, eluting with a gradient of 0-40% EtOAc in heptane, to provide Intermediate 3-074.2. MS (ESI) m/z: 555.2 (M+H)⁺.

Step 3: tert-Butyl 4-((2R,3R)-2-methyl-1-(6-(4-methylene-1-oxa-8-azaspiro[4.5]decan-8-yl)-2-(trifluoromethyl)pyrimidin-4-yl)azetidin-3-yl)piperazine-1-carboxylate, Intermediate 3-074.3. Methyltriphenylphosphonium bromide (386 mg, 1.08 mmol) and THF (5 mL) were cooled to 0° C. To the reaction mixture was added NaHMDS (1.0 M in THE, 1.08 mL, 1.08 mmol) and the reaction mixture was stirred at rt for 30 min. To the reaction mixture was added a solution of Intermediate 3-074.2 (300 mg, 0.54 mmol) in THF (1 mL) and the reaction mixture was stirred at rt for 2 h. The reaction mixture was quenched by the addition sat. aq. NH₄Cl and extracted with EtOAc. The organic extracts were washed with brine, dried over Na₂SO₄ filtered, and concentrated in vacuo. The crude material was purified by silica gel chromatography, eluting with a gradient of 0-40% EtOAc in heptane, to provide Intermediate 3-074.3. MS (ESI) m/z: 553.2 (M+H)⁺.

Step 4: 8-(6-((2R,3R)-2-Methyl-3-(piperazin-1-yl)azetidin-1-yl)-2-(trifluoromethyl)pyrimidin-4-yl)-4-methylene-1-oxa-8-azaspiro[4.5]decane, Intermediate 3-074.4. To a solution of Intermediate 3-074.3 (229 mg, 0.42 mmol) in DCM (3 mL) was added TFA (0.5 mL, 0.42 mmol) and the reaction mixture was stirred for 30 min at rt. The reaction mixture was concentrated to provide Intermediate 3-074.4 as a TFA salt that was used directly in the next step. MS (ESI) m/z: 453.2 (M+H)⁺.

Step 5: 1-(4-((2R,3R)-2-Methyl-1-(6-(4-methylene-1-oxa-8-azaspiro[4.5]decan8-yl)-2-(trifluoromethyl)pyrimidin-4-yl)azetidin-3-yl)piperazin-1-yl) prop-2-en-1-one. Compound 3-074. The reaction mixture from Step 4 was redissolved in DCM (3 mL) and added DIPEA (0.72 mL, 4.15 mmol) followed by acryloyl chloride (0.2 M in DCM, 2.1 mL, 0.415 mmol). The reaction mixture was stirred at rt for 30 min. The reaction mixture was quenched by the addition of sat. aq. NaHCO₃ and extracted with DCM. The combined organic extracts were washed with sat. aq. NH₄Cl, dried over Na₂SO₄, and concentrated in vacuo. The crude material was purified by silica gel chromatography, eluting with a gradient of 0-30% EtOAc/EtOH (3:1) in heptane to provide Compound 3-074. MS (ESI) m/z: 507.1 (M+H)⁺. ¹H NMR (400 MHz, CDCl₃) δ 6.48-6.63 (m, 1H), 6.24-6.35 (m, 1H), 5.67-5.74 (m, 1H), 5.20-5.26 (m, 1H), 4.97-5.03 (m, 1H), 4.76-4.82 (m, 1H), 4.48-4.60 (m, 1H), 4.18-4.31 (m, 2H), 3.84-4.02 (m, 4H), 3.57-3.79 (m, 4H), 3.24-3.33 (m, 1H), 3.14-3.23 (m, 2H), 2.61-2.80 (m, 2H), 2.26-2.45 (m, 4H), 1.68-1.75 (m, 2H), 1.59-1.67 (m, 2H), 1.45-1.50 (m, 3H). ¹⁹F NMR (376 MHz, CDCl₃) δ −76.63-−68.82 (m, 3F).

### Example 3-075:4-(3-(4-Acryloylpiperazin-1-yl)azetidin-1-yl)-6-(1-methyl-2-oxo-1,8-diazaspiro[4.5]decan-8-yl)-2-(trifluoromethyl)nicotinonitrile

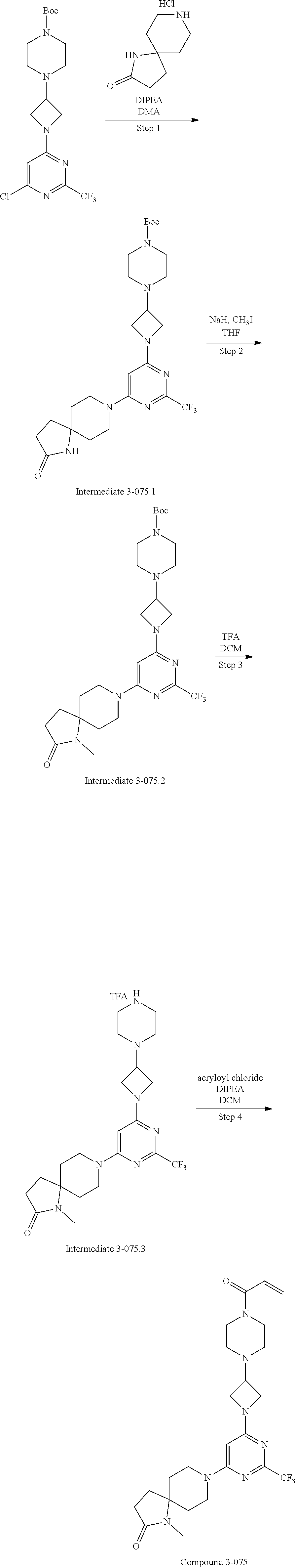

Step 1: tert-butyl 4-(1-(6-(2-oxo-1,8-diazaspiro[4.5]decan-8-yl)-2-(trifluoromethyl)pyrimidin-4-yl)azetidin-3-yl)piperazine-1-carboxylate, Intermediate 3-075.1, 1,8-Diazaspiro[4.5]decan-2-one hydrochloride (300 mg, 1.57 mmol), tert-butyl 4-(1-(6-chloro-2-(trifluoromethyl)pyrimidin-4-yl)azetidin-3-yl) piperazine-1-carboxylate (631 mg, 1.42 mmol), DIPEA (1.37 mL, 7.87 mmol), and DMA (5.3 mL) were stirred at 90° C. for 96 h. The reaction was quenched with sat. aq. NH₄Cl and extracted with EtOAc. The organic extract was washed with H₂O, sat. aq. NH₄Cl (2×), and the combined aqueous phase was extracted with EtOAc. The combined organic extracts were washed with brine, dried over Na₂SO₄, and concentrated. The crude material was purified by silica gel chromatography, eluting with a gradient of 0-6% MeOH in DCM, to provide Intermediate 3-075.1. MS (ESI) m/z: 564.0 (M+H)⁺.

Step 2: tert-butyl 4-(1-(6-(1-methyl-2-oxo-1,8-diazaspiro[4.5]decan-8-yl)-2-(trifluoromethyl)pyrimidin-4-yl)azetidin-3-yl)piperazine-1-carboxylate, Intermediate 3-075.2. Sodium hydride (60 wt % in mineral oil, 43 mg, 1.07 mmol) and Intermediate 3-075.1 (200 mg, 0.36 mmol) in THF (2.2 mL) were stirred at rt. After 15 min. MeI (44 μL, 0.71 mmol) was added and the reaction mixture stirred for 2 h. An additional 2 equiv. MeI was added, and the reaction mixture was stirred for 48 h. The reaction was quenched with sat. aq. NH₄Cl solution. The crude material was extracted with EtOAc (3×), washed with brine, dried over Na₂SO₄, and concentrated. The crude material was purified by silica gel chromatography, eluting with a gradient of 0-10% MeOH in DCM, to provide Intermediate 3-075.2. MS (ESI) m/z: 578.4 (M+H)⁺. ¹H NMR. (400 MHz, CDCl₃) δ 5.42 (s, 1H), 4.33-4.48 (m, 4H), 4.12 (br s, 2H), 3.49 (br t, J=4.6 Hz, 4H), 3.27-3.42 (m, 1H), 2.97-3.06 (m, 2H), 2.75 (s, 3H), 2.46 (t, J=8.0 Hz, 2H), 2.37 (br s, 4H), 2.05 (t, J=8.0 Hz, 2H), 1.94 (td, J=13.0, 4.6 Hz, 2H), 1.46-1.55 (m, 11H).

Step 3: 1-Methyl-8-(6-(3-(piperazin-1-yl)azetidin-1-yl)-2-(trifluoromethyl)pyrimidin-4-yl)-1,8-diazaspiro[4.5]decan-2-one, Intermediate 3-075.3. Intermediate 3-075.2 (47.8 mg, 0.083 mmol) and TFA (123 μL, 1.65 mmol) in DCM (0.9 mL) were stirred at rt for 1 h. The reaction mixture was concentrated to provide Intermediate 3-075.3 as a TFA salt, which was used in the next step. MS (ESI) m/z: 478.2 (M+H)⁺.

Step 4: 4-(3-(4-Acryloylpiperazin-1-yl)azetidin-1-yl)-6-(1-methyl-2-oxo-1,8-diazaspiro[4.5]decan-8-yl)-2-(trifluoromethyl)nicotinonitrile, Compound 3-075. To the reaction mixture from Step 3 was added 0.5 mL DCM followed by DIPEA (0.15 mL, 0.83 mmol) and acryloyl chloride (0.2 M in DCM, 0.54 mL, 0.11 mmol) at rt and stirred for 15 min. The reaction solution was quenched and washed with sat. aq. NH₄Cl solution and brine. The organic phase was dried over Na₂SO₄ and concentrated. The crude material was purified by silica gel chromatography, eluting with a gradient of 0-8% MeOH in DCM, to provide Compound 3-075. MS (ESI) m/z: 532.2 (M+H)⁺. ¹H NMR (400 MHz, CDCl₃) δ 6.57 (dd, J=16.8, 10.6 Hz, 1H), 6.32 (dd, J=16.9, 1.9 Hz, 1H), 5.74 (dd, J=10.6, 1.8 Hz, 1H), 5.43 (s, 1H), 4.29-4.54 (m, 4H), 4.14 (br s, 2H), 3.75 (br s, 2H), 3.64 (br s, 2H), 3.28-3.39 (m, 1H), 2.97-3.09 (m, 2H), 2.73-2.83 (m, 3H), 2.40-2.51 (m, 6H), 2.06 (t, J=8.0 Hz, 2H), 1.94 (td, J=12.9, 4.5 Hz, 2H), 1.53 (br d, J=12.8 Hz, 2H), ¹⁹F NMR (376 MHz, CDCl₃) δ −66.74 (s, 3F).

### Example 3-076:1-(4-((2R,3R)-2-Methyl-1-(6-(3′-methyl-6′,7′-dihydrospiro[piperidine-4,4′-pyrazolo[5,1-c][1,4]oxazin]-1-yl)-2-(trifluoromethyl)pyrimidin-4-yl)azetidin-3-yl)piperazin-1-yl)prop-2-en-1-one

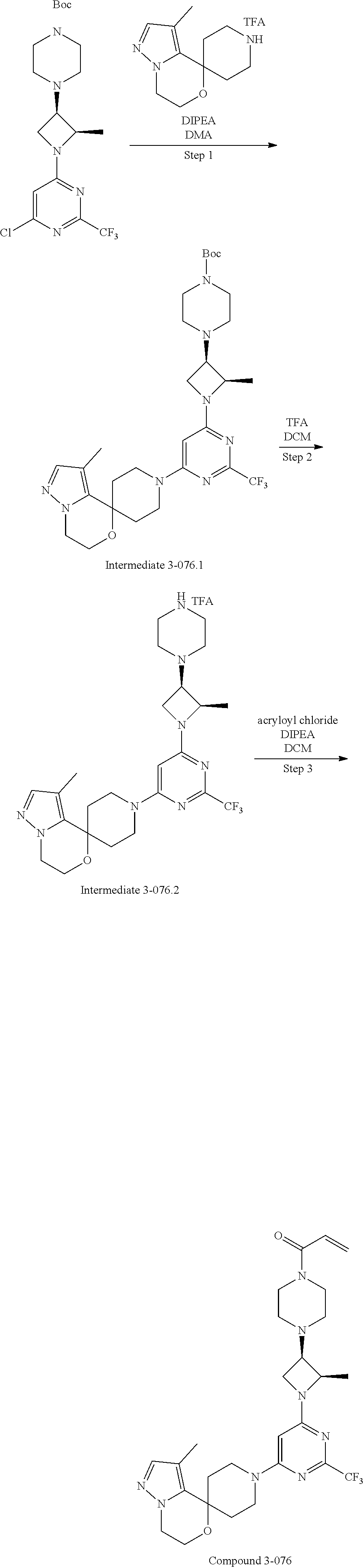 [SMILES: C[C@@H]1[C@H](N2CCNCC2)CN1c1cc(Cl)nc(C(F)(F)F)n1 ; Cc1cnn2c1C1(CCNCC1)OCC2 ; Cc1cnn2c1C1(CCN(c3cc(N4C[C@@H](N5CCN(C(=O)OC(C)(C)C)CC5)[C@H]4C)nc(C(F)(F)F)n3)CC1)OCC2 ; Cc1cnn2c1C1(CCN(c3cc(N4C[C@@H](N5CCNCC5)[C@H]4C)nc(C(F)(F)F)n3)CC1)OCC2 ; "Boc" ; C=CC(=O)N1CCN([C@@H]2CN(c3cc(N4CCC5(CC4)OCCn4ncc(C)c45)nc(C(F)(F)F)n3)[C@@H]2C)CC1]

Step 1: tert-Butyl 4-((2R,3R)-2-methyl-1-(6-(3′-methyl-6′,7′-dihydrospiro[piperidine-4,4′-pyrazolo[5,1-c][1,4]oxazin]-1-yl)-2-(trifluoromethyl)pyrimidin-4-yl)azetidin-3-yl)piperazine-1-carboxylate. Intermediate 3-076.1. To a solution of 3′-methyl-6′,7′-dihydrospiro[piperidine-4,4′-pyrazolo[5,1-c][1,4]oxazine] (250 mg, 0.82 mmol. TFA salt) and DIPEA (1.43 mL, 8.19 mmol) in DMA (10 mL) was added tert-butyl 4-((2R,3R)-1-(6-chloro-2-(trifluoromethyl)pyrimidin-4-yl)-2-methylazetidin-3-yl)piperazine-1-carboxylate (393 mg, 0.90 mmol). The mixture was stirred at 100° C. for 12 h under Ne atmosphere. The reaction mixture was quenched by addition of 30 mL H₂O, and then extracted with EtOAc (30 mL×3). The combined organic extracts were washed with brine (20 mL×2 times), dried over Na₂SO₄, filtered, and concentrated under reduced pressure. The residue was purified by silica gel chromatography, eluting with a gradient of 0% to 33% EtOAc in petroleum ether to provide Intermediate 3-076.1. MS (ESI) m/z: 607.4 (M+H)⁺.

Step 2: 3′-Methyl-1-(6-((2R,3R)-2-methyl-3-(piperazin-1-yl)azetidin-1-yl)-2-(trifluoromethyl)pyrimidin-4-yl)-6′,7′-dihydrospiro[piperidine-4,4′-pyrazolo[5,1-c][1,4]oxazine] (TFA salt), Intermediate 3-076.2. To a solution of Intermediate 3-076.1 (210 mg, 0.35 mmol) in DCM (5 mL) was added TFA (1 mL). The mixture was stirred at 20° C. for 2 h under N₂ atmosphere. The reaction mixture was concentrated under reduced pressure to give Intermediate 3-076.2, as a TFA salt. MS (ESI) m/z: 507.3 (M+H)⁺.

Step 3: 1-(4-((2R,3R)-2-Methyl-1-(6-(3′-methyl-6′,7′-dihydrospiro[piperidine-4,4′-pyrazolo[5,1-c][1,4]oxazin]-1-yl)-2-(trifluoromethyl)pyrimidin-4-yl)azetidin-3-yl)piperazin-1-yl)prop-2-en-1-one, Compound 3-076. To a solution of Intermediate 3-076.2 (210 mg, 0.35 mmol, TFA salt) and DIPEA (0.61 ml, 3.47 mmol) in DCM (5 mL) was added acryloyl chloride (31 mg, 0.35 mmol) at 0° C. The mixture was stirred at 0° C. for 0.5 h under N₂ atmosphere. The reaction mixture was concentrated under reduced pressure. The residue was purified by prep-HPLC [H₂O (NH₄HCO₃)-ACN] to provide Compound 3-076. MS (ESI) m/z: 561.5 (M+H)⁺. ¹H NMR (400 MHz, MeOD) δ 7.23 (s, 1H) 6.70-6.80 (m, 1H) 6.15-6.25 (m, 1H) 5.70-5.80 (m, 1H) 5.53 (s, 1H) 4.50-4.65 (m, 1H) 4.25-4.35 (d, J=12.00 Hz, 2H) 4.05-4.18 (m, 4H) 3.87-4.02 (m, 2H) 3.63-3.72 (m, 4H) 3.22-3.35 (m, 2H) 2.40 (s, 4H) 2.05-2.15 (m, 2H) 2.04 (s, 3H) 1.90-2.00 (m, 2H) 1.45-1.55 (d, J=6.40 Hz, 3H).

Examples in the table below were prepared in a manner similar to that described above.

TABLE 2-9

| | | | MS (ESI) |
|---|---|---|---|
| Compound | | | m/z: |
| No. | Chemical Structure | Name | (M + H)⁺ |
| 3-077 | 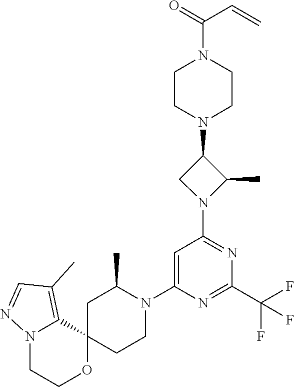 [SMILES: C=CC(=O)N1CCN([C@@H]2CN(c3cc(N4CC[C@]5(C[C@H]4C)OCCn4ncc(C)c45)nc(C(F)(F)F)n3)[C@@H]2C)CC1] | 1-(4-((2R,3R)-1-(6-((2R,4R)- 2,3′-dimethyl-6′,7′-dihydro- 1H-spiro[piperidine-4,4′- pyrazolo[5,1-c][1,4]oxazin]-1- yl)-2-(trifluoromethyl)-4- pyrimidinyl)-2-methyl-3- azetidinyl)-1-piperazinyl)-2- propen-1-one | 575.3 |
| 3-078 | 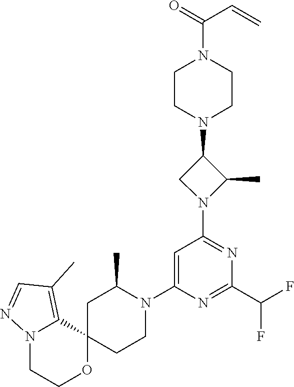 [SMILES: C=CC(=O)N1CCN([C@@H]2CN(c3cc(N4CC[C@]5(C[C@H]4C)OCCn4ncc(C)c45)nc(C(F)F)n3)[C@@H]2C)CC1] | 1-(4-((2R,3R)-1-(2- (difluoromethyl)-6-((2R,4R)- 2,3′-dimethyl-6′,7′-dihydro- 1H-spiro[piperidine-4,4′- pyrazolo[5.1-c][1,4]oxazin]-1- yl)-4-pyrimidinyl)-2-methyl-3- azetidinyl)-1-piperazinyl)-2- propen-1-one | 557.1 |
| 3-079 | 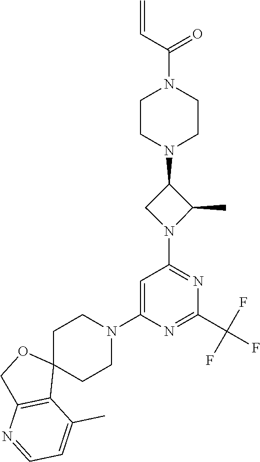 [SMILES: C=CC(=O)N1CCN([C@@H]2CN(c3cc(N4CCC5(CC4)OCc4nccc(C)c45)nc(C(F)(F)F)n3)[C@@H]2C)CC1] | 1-(4-((2R,3R)-2-methyl-1-(6- (4-methyl-1′H,7H- spiro[furo[3,4-b]pyridine-5,4′- piperidin]-1′-yl)-2- (trifluoromethyl)-4- pyrimidinyl)-3-azetidinyl)-1- piperazinyl)-2-propen-1-one | 558.2 |
| 3-080 | 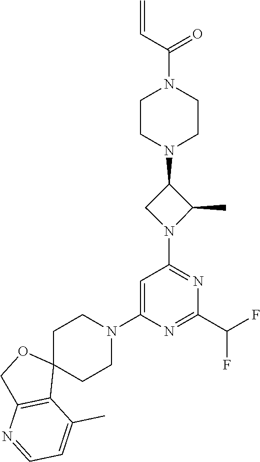 [SMILES: C=CC(=O)N1CCN([C@@H]2CN(c3cc(N4CCC5(CC4)OCc4nccc(C)c45)nc(C(F)F)n3)[C@@H]2C)CC1] | 1-(4-((2R,3R)-1-(2- (difluoromethyl)-6-(4-methyl- 7H-spiro[furo][3,4-b]pyridine- 5,4′-piperidin]-1′-yl)pyrimidin- 4-yl)-2-methylazetidin-3- yl)piperazin-1-yl)prop-2-en-1- one | 540.1 |
| 3-081 | 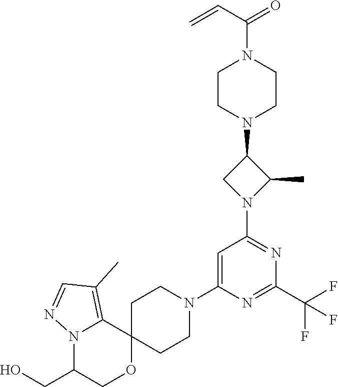 [SMILES: C=CC(=O)N1CCN([C@@H]2CN(c3cc(N4CCC5(CC4)OCC(CO)n4ncc(C)c45)nc(C(F)(F)F)n3)[C@@H]2C)CC1] | 1-(4-((2R,3R)-1-(6-(7′- (hydroxymethyl)-3′-methyl- 6′,7′-dihydrospiro[piperidine- 4,4′-pyrazolo[5,1- c][1.4]oxazin]-1-yl)-2- (trifluoromethyl)pyrimidin-4- yl)-2-methylazetidin-3- yl)piperazin-1-yl)prop-2-en-1- one | 591.1 |
| 3-082 | 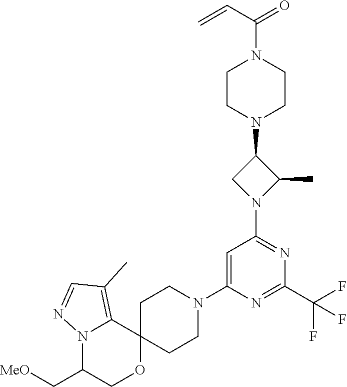 [SMILES: C=CC(=O)N1CCN([C@@H]2CN(c3cc(N4CCC5(CC4)OCC(COC)n4ncc(C)c45)nc(C(F)(F)F)n3)[C@@H]2C)CC1] | 1-(4-((2R,3R)-1-(6-(7′- (methoxymethyl)-3′-methyl- 6′,7′-dihydrospiro[piperidine- 4,4′-pyrazolo[5,1- c][1,4]oxazin]-1-yl)-2- (trifluoromethyl)pyrimidin-4- yl)-2-methylazetidin-3- yl)piperazin-1-yl)prop-2-en-1- one | 605.0 |
| 3-083 | 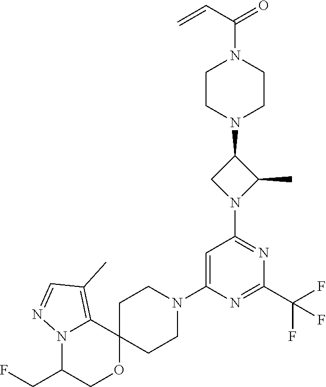 [SMILES: C=CC(=O)N1CCN([C@@H]2CN(c3cc(N4CCC5(CC4)OCC(CF)n4ncc(C)c45)nc(C(F)(F)F)n3)[C@@H]2C)CC1] | 1-(4-((2R,3R)-1-(6-(7′- (fluoromethyl)-3′-methyl- 6′,7′-dihydrospiro[piperidine- 4,4′-pyrazolo[5,1- c][1,4]oxazin]-1-yl)-2- (trifluoromethyl)pyrimidin-4- yl)-2-methylazetidin-3- yl)piperazin-1-yl)prop-2-en-1- one | 593.0 |
| 3-084 | 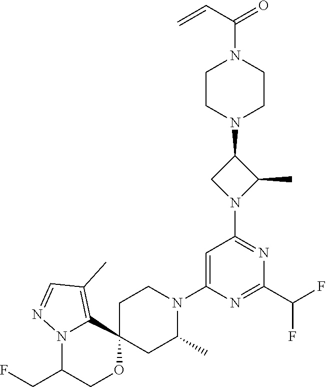 | 1-(4-((2R,3R)-1-(2- (difluoromethyl)-6-((2R,4R)- 7′-(fluoromethyl)-2,3′- dimethyl-6′,7′- dihydrospiro[piperidine-4,4′- pyrazolo[5,1-c][1,4]oxazin]-1- yl)pyrimidin-4-yl)-2- methylazetidin-3-yl)piperazin- 1-yl)prop-2-en-1-one | 589.0 |
| 3-085 | 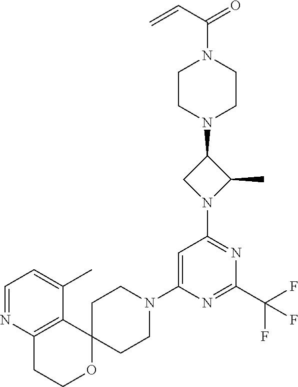 [SMILES: C=CC(=O)N1CCN([C@@H]2CN(c3cc(N4CCC5(CC4)OCCc4nccc(C)c45)nc(C(F)(F)F)n3)[C@@H]2C)CC1] | 1-(4-((2R,3R)-2-methyl-1-(6- (4′-methyl-7′,8′- dihydrospiro[piperidine-4,5′- pyrano[4,3-b]pyridin]-1-yl)-2- (trifluoromethyl)pyrimidin-4- yl)azetidin-3-yl)piperazin-1- yl)prop-2-en-1-one | 571.2 |
| 3-086 | 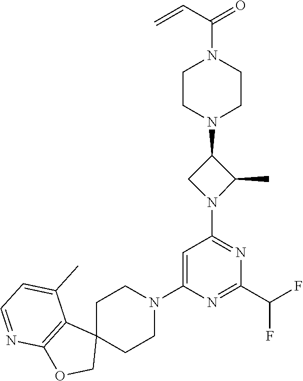 [SMILES: C=CC(=O)N1CCN([C@@H]2CN(c3cc(N4CCC5(CC4)COc4nccc(C)c45)nc(C(F)F)n3)[C@@H]2C)CC1] | 1-(4-((2R,3R)-1-(2- (difluoromethyl)-6-(4-methyl- 2H-spiro[furo[2.3-b]pyridine- 3,4′-piperidin]-1′-yl)pyrimidin- 4-yl)-2-methylazetidin-3- yl)piperazin-1-yl)prop-2-en-1- one | 540.2 |
| 3-092 | 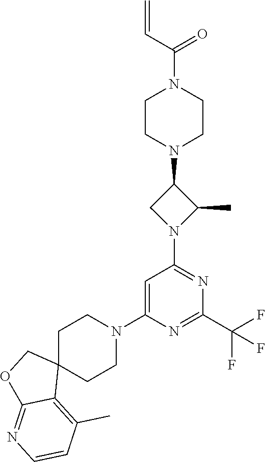 [SMILES: C=CC(=O)N1CCN([C@@H]2CN(c3cc(N4CCC5(CC4)COc4nccc(C)c45)nc(C(F)(F)F)n3)[C@@H]2C)CC1] | 1-(4-((2R,3R)-2-methyl-1-(6- (4-methyl-1′H-spiro[furo[2,3- b]pyridine-3,4′-piperidin]-1′- yl)-2-(trifluoromethyl)-4- pyrimidinyl)-3-azetidinyl)-1- piperazinyl)-2-propen-1-one | 558.2 |
| 3-093 | 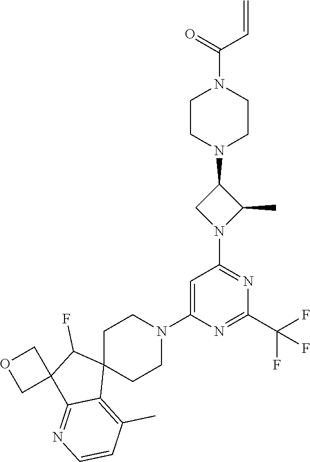 [SMILES: C=CC(=O)N1CCN([C@@H]2CN(c3cc(N4CCC5(CC4)c4c(C)ccnc4C4(COC4)C5F)nc(C(F)(F)F)n3)[C@@H]2C)CC1] | 1-(4-((2R,3R)-1-(6-(6′-fluoro- 4′-methyl-1″H- dispiro[oxetane-3,7′- cyclopenta[b]pyridine-5′,4″- piperidin]-1″-yl)-2- (trifluoromethyl)-4- pyrimidinyl)-2-methyl-3- azetidinyl)-1-piperazinyl)-2- propen-1-one | 616.1 |
| 3-094 | 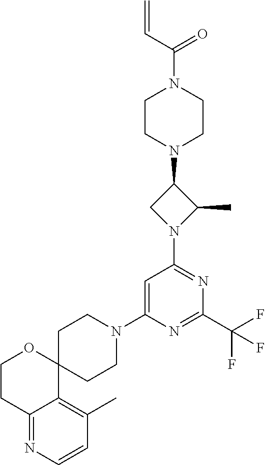 [SMILES: C=CC(=O)N1CCN([C@@H]2CN(c3cc(N4CCC5(CC4)OCCc4nccc(C)c45)nc(C(F)(F)F)n3)[C@@H]2C)CC1] | 1-(4-((2R,3R)-2-methyl-1-(6- (4′-methyl-7′,8′-dihydro-1H- spiro[piperidine-4,5′- pyrano[4.3-b]pyridin]-1-yl)-2- (trifluoromethyl)-4- pyrimidinyl)-3-azetidinyl)-1- piperazinyl)-2-propen-1-one | 572.4 |
| 3-095 | 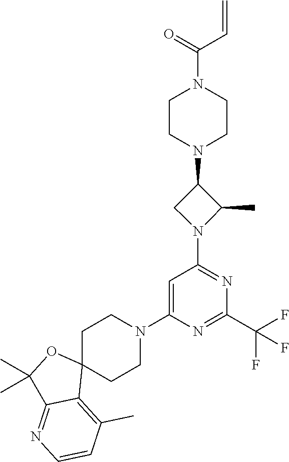 [SMILES: C=CC(=O)N1CCN([C@@H]2CN(c3cc(N4CCC5(CC4)OC(C)(C)c4nccc(C)c45)nc(C(F)(F)F)n3)[C@@H]2C)CC1] | 1-(4-((2R,3R)-2-methyl-1-(2- (trifluoromethyl)-6-(4,7,7- trimethyl-1′H,7H- spiro[furo][3.4-b]pyridine-5,4′- piperidin]-1′-yl)-4- pyrimidinyl)-3-azetidinyl)-1- piperazinyl)-2-propen-1-one | 586.0 |
| 3-096 | 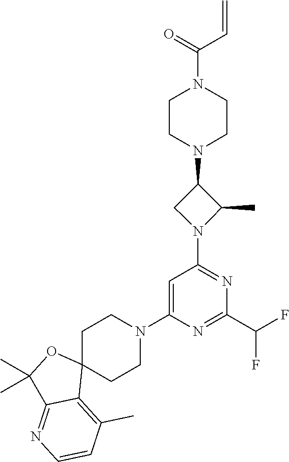 [SMILES: C=CC(=O)N1CCN([C@@H]2CN(c3cc(N4CCC5(CC4)OC(C)(C)c4nccc(C)c45)nc(C(F)F)n3)[C@@H]2C)CC1] | 1-(4-((2R,3R)-1-(2- (difluoromethyl)-6-(4,7,7- trimethyl-1′H,7H- spiro[furo[3,4-b]pyridine-5,4′- piperidin]-1′-yl)-4- pyrimidinyl)-2-methyl-3- azetidinyl)-1-piperazinyl)-2- propen-1-one | 568.0 |
| 3-097 | 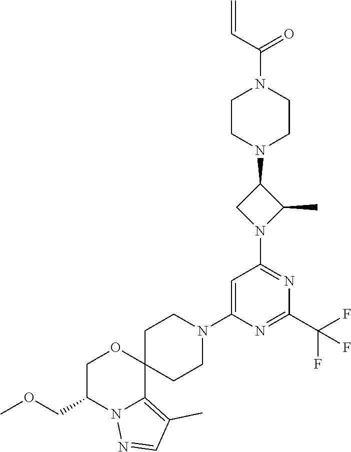 [SMILES: C=CC(=O)N1CCN([C@@H]2CN(c3cc(N4CCC5(CC4)OC[C@@H](COC)n4ncc(C)c45)nc(C(F)(F)F)n3)[C@@H]2C)CC1] | 1-(4-((2R,3R)-1-(6-((7′R)-7′- (methoxymethyl)-3′-methyl- 6′,7′-dihydro-1H- spiro[piperidine-4,4′- pyrazolo[5,1-c][1.4]oxazin]-1- yl)-2-(trifluoromethyl)-4- pyrimidinyl)-2-methyl-3- azetidinyl)-1-piperazinyl)-2- propen-1-one | 605.1 |
| 3-098* | 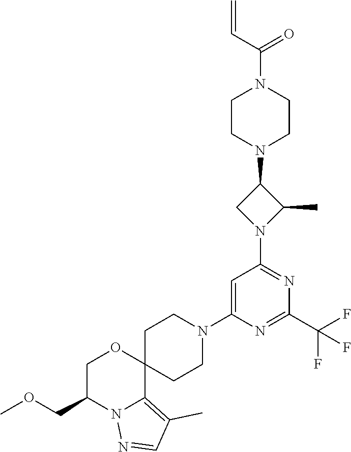 [SMILES: C=CC(=O)N1CCN([C@@H]2CN(c3cc(N4CCC5(CC4)OC[C@H](COC)n4ncc(C)c45)nc(C(F)(F)F)n3)[C@@H]2C)CC1] | 1-(4-(2R,3R)-1-(6-((7′S)-7′- (methoxymethyl)-3′-methyl- 6′,7′-dihydro-1H- spiro[piperidine-4.4′- pyrazolo[5.1-c][1,4]oxazin]-1- yl)-2-(trifluoromethyl)-4- pyrimidinyl)-2-methyl-3- azetidinyl)-1-piperazinyl)-2- propen-1-one | 605.1 |
| 3-099 | 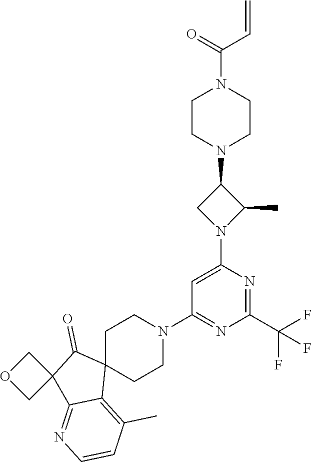 [SMILES: C=CC(=O)N1CCN([C@@H]2CN(c3cc(N4CCC5(CC4)C(=O)C4(COC4)c4nccc(C)c45)nc(C(F)(F)F)n3)[C@@H]2C)CC1] | 4′-methyl-1″-(6-((2R,3R)-2- methyl-3-(4-(2-propenoyl)-1- piperazinyl)-1-azetidinyl)-2- (trifluoromethyl)-4- pyrimidinyl)dispiro[oxetane- 3,7′-cyclopenta[b]pyridine- 5′,4″-piperidin]-6′-one | 612.1 |
| 3-100 | 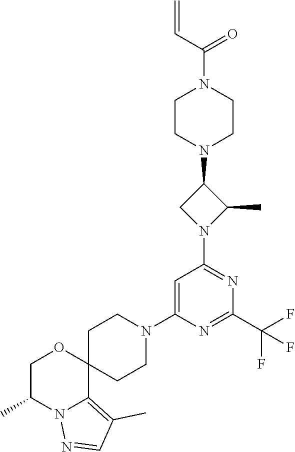 [SMILES: C=CC(=O)N1CCN([C@@H]2CN(c3cc(N4CCC5(CC4)OC[C@@H](C)n4ncc(C)c45)nc(C(F)(F)F)n3)[C@@H]2C)CC1] | 1-(4-((2R,3R)-1-(6-((7′R)-3′,7′- dimethyl-6′,7′-dihydro-1H- spiro[piperidine-4,4′- pyrazolo[5,1-c][1,4]oxazin]-1- yl)-2-(trifluoromethyl)-4- pyrimidinyl)-2-methyl-3- azetidinyl)-1-piperazinyl)-2- propen-1-one | 575.1 |
| 3-101 | 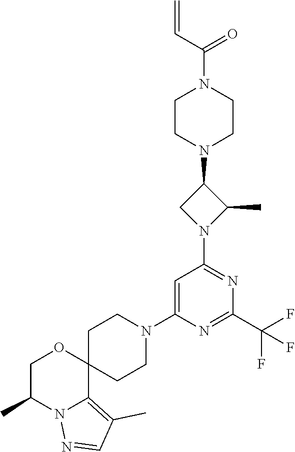 [SMILES: C=CC(=O)N1CCN([C@@H]2CN(c3cc(N4CCC5(CC4)OC[C@H](C)n4ncc(C)c45)nc(C(F)(F)F)n3)[C@@H]2C)CC1] | 1-(4-((2R,3R)-1-(6-((7′S)-3′,7′- dimethyl-6′,7′-dihydro-1H- spiro[piperidine-4,4′- pyrazolo[5.1-c][1,4]oxazin]-1- yl)-2-(trifluoromethyl)-4- pyrimidinyl)-2-methyl-3- azetidinyl)-1-piperazinyl)-2- propen-1-one | 575.1 |
| 3-102 | 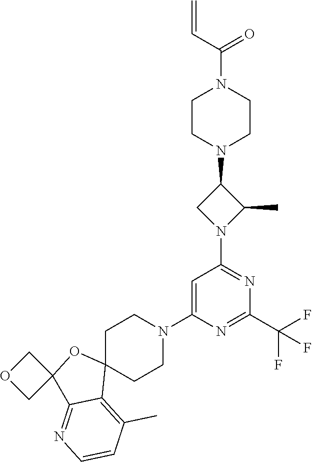 [SMILES: C=CC(=O)N1CCN([C@@H]2CN(c3cc(N4CCC5(CC4)OC4(COC4)c4nccc(C)c45)nc(C(F)(F)F)n3)[C@@H]2C)CC1] | 1-(4-((2R,3R)-2-methyl-1-(6- (4′-methyl-1″H- dispiro[oxetane-3,7′-furo[3,4- b]pyridine-5′.4″-piperidin]-1″- yl)-2-(trifluoromethyl)-4- pyrimidinyl)-3-azetidinyl)-1- piperazinyl)-2-propen-1-one | 600.0 |
| 3-103 | 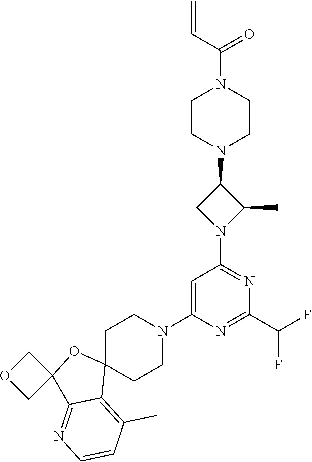 [SMILES: C=CC(=O)N1CCN([C@@H]2CN(c3cc(N4CCC5(CC4)OC4(COC4)c4nccc(C)c45)nc(C(F)F)n3)[C@@H]2C)CC1] | 1-(4-((2R,3R)-1-(2- (difluoromethyl)-6-(4′-methyl- 1″H-dispiro[oxetane-3,7′- furo[3,4-b]pyridine-5′,4″- piperidin]-1″-yl)-4- pyrimidinyl)-2-methyl-3- azetidinyl)-1-piperazinyl)-2- propen-1-one | 582.0 |
| 3-104 | 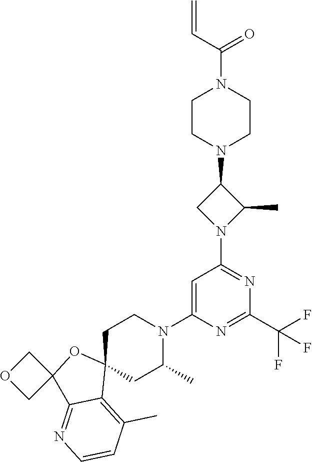 [SMILES: C=CC(=O)N1CCN([C@@H]2CN(c3cc(N4CC[C@]5(C[C@H]4C)OC4(COC4)c4nccc(C)c45)nc(C(F)(F)F)n3)[C@@H]2C)CC1] | 1-(4-((2R,3R)-1-(6-((2″R,5′R)- 2″,4′-dimethyl-1″H- dispiro[oxetane-3,7′-furo[3,4- b]pyridine-5′,4″-piperidin]-1″- yl)-2-(trifluoromethyl)-4- pyrimidinyl)-2-methyl-3- azetidinyl)-1-piperazinyl)-2- propen-1-one | 614.1 |

*SFC | separation condition: Column-Chiralpak IG (2 × 25 μm). Mobile phase: 35% 1:1 ACN:MeOH Flowrate: 80 mL/min

Examples in the table below were prepared in a manner similar to that described above and with an additional final step of chiral separation by SFC. The stereochemistry with (*) was assigned arbitrarily.

TABLE 2-10

| | | | MS (ESI) | SFC \| |
|---|---|---|---|---|
| Cmpd | | | m/z: | separation |
| No. | Chemical Structure | Name | (M + H)⁺ | condition |
| 3-087-1 | 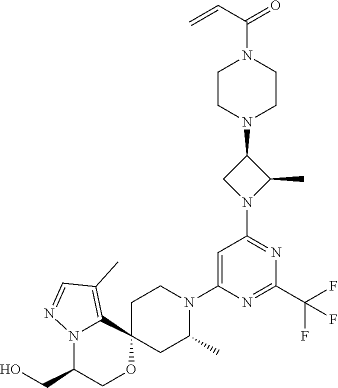 [SMILES: C=CC(=O)N1CCN([C@@H]2CN(c3cc(N4CC[C@]5(C[C@H]4C)OC[C@@H](CO)n4ncc(C)c45)nc(C(F)(F)F)n3)[C@@H]2C)CC1] | Peak 1: 1-(4-((2R,3R)-1-(6- ((2R,4R,7″R)-7′- (hydroxymethyl)-2,3′- dimethyl-6′,7′- dihydrospiro[piperidine- 4,4′-pyrazolo[5,1- c][1,4]oxazin]-1-yl)-2- (trifluoromethyl)pyrimidin- 4-yl)-2-methylazetidin-3- yl)piperazin-1-yl)prop-2- en-1-one | 605.0 | Column: Chiralcel OD (2 × 25 cm 5 μm). Mobile phase: 35% MeOH Flowrate: 80 mL/min. |
| 3-087-2 | 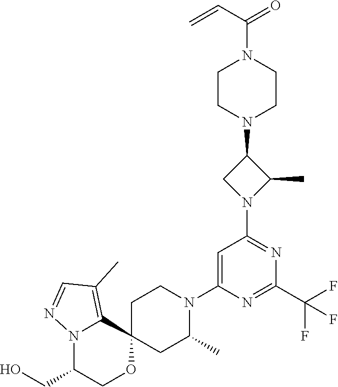 [SMILES: C=CC(=O)N1CCN([C@@H]2CN(c3cc(N4CC[C@]5(C[C@H]4C)OC[C@H](CO)n4ncc(C)c45)nc(C(F)(F)F)n3)[C@@H]2C)CC1] | Peak 2: 1-(4-((2R,3R)-1- (6-((2R,4R,7′S)-7′- (hydroxymethyl)-2,3′- dimethyl-6′,7′- dihydrospiro\|piperidine- 4,4′-pyrazolo[5,1- c][1,4]oxazin]-1-yl)-2- (trifluoromethyl)pyrimidin- 4-yl)-2-methylazetidin-3- yl)piperazin-1-yl)prop-2- en-1-one | 605.0 | Column: Chiralcel OD (2 × 25 cm 5 μm). Mobile phase: 35% MeOH Flowrate: 80 mL/min. |
| 3-088-1 | 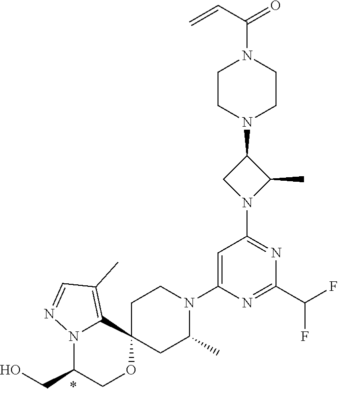 [SMILES: C=CC(=O)N1CCN([C@@H]2CN(c3cc(N4CC[C@]5(C[C@H]4C)OC[C@@H](CO)n4ncc(C)c45)nc(C(F)F)n3)[C@@H]2C)CC1] | Peak 1: 1-(4-((2R,3R)-1-(2- (difluoromethyl)-6- ((2R,4R,7′R)-7′- (hydroxymethyl)-2,3′- dimethyl-6′,7′- dihydrospiro[piperidine- 4.4′-pyrazolo[5.1- c][1,4]oxazin]-1- yl)pyrimidin-4-yl)-2- methylazetidin-3- yl)piperazin-1-yl)prop-2- en-1-one | 587.0 | Column: Chiralcel OD (21 × 250 mm, 5 μm). Mobile phase: 35% MeOH Flowrate: 80 mL/min. |
| 3-088-2 | 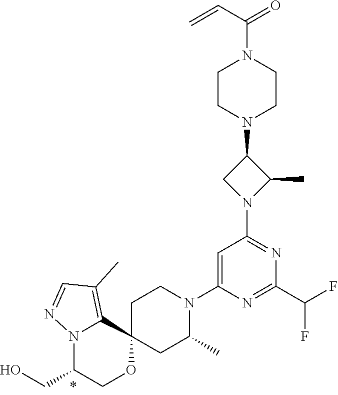 [SMILES: C=CC(=O)N1CCN([C@@H]2CN(c3cc(N4CC[C@]5(C[C@H]4C)OC[C@H](CO)n4ncc(C)c45)nc(C(F)F)n3)[C@@H]2C)CC1] | Peak 2: 1-(4-((2R,3R)-1-(2- (difluoromethyl)-6- ((2R,4R,7′S)-7′- (hydroxymethyl)-2,3′- dimethyl-6′,7′- dihydrospiro[piperidine- 4,4′-pyrazolo[5,1- c][1,4]oxazin]-1- yl)pyrimidin-4-yl)-2- methylazetidin-3- yl)piperazin-1-yl)prop-2- en-1-one | 587.0 | Column: Chiralcel OD (21 × 250 mm, 5 μm). Mobile phase: 35% MeOH Flowrate: 80 mL/min. |
| 3-089-1 | 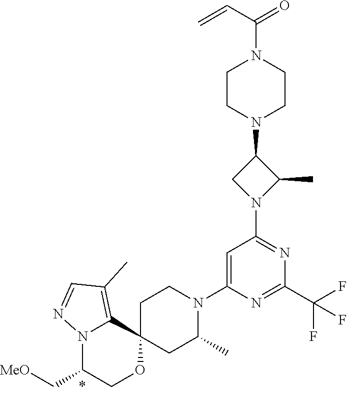 [SMILES: C=CC(=O)N1CCN([C@@H]2CN(c3cc(N4CC[C@]5(C[C@H]4C)OC[C@H](COC)n4ncc(C)c45)nc(C(F)(F)F)n3)[C@@H]2C)CC1] | Peak 1: 1-(4-((2R,3R)-1-(6- ((2R,4R,7′S)-7′- (methoxymethyl)-2,3′- dimethyl-6′,7′- dihydrospiro[piperidine- 4,4′-pyrazolo[5,1- c][1,4]oxazin]-1-yl)-2- (trifluoromethyl)pyrimidin- 4-yl)-2-methylazetidin-3- yl)piperazin-1-yl)prop-2- en-1-one | 619.0 | Column; Chromega Chiral CC4 (21 × 150 mm, 5 μm). Mobile phase: 35% MeOH Flowrate: 80 mL/min. |
| 3-090-1 | 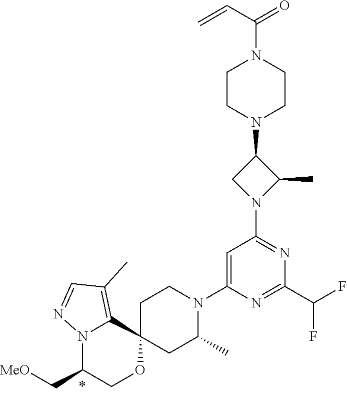 [SMILES: C=CC(=O)N1CCN([C@@H]2CN(c3cc(N4CC[C@]5(C[C@H]4C)OC[C@@H](COC)n4ncc(C)c45)nc(C(F)F)n3)[C@@H]2C)CC1] | Peak 1: 1-(4-((2R,3R)-1-(2- (difluoromethyl)-6- ((2R,4R,7′R)-7′- (methoxymethyl)-2,3′- dimethyl-6′,7′- dihydrospiro[piperidine- 4,4′-pyrazolo[5,1- c][1,4]oxazin]-1- yl)pyrimidin-4-yl)-2- methylazetidin-3- yl)piperazin-1-yl)prop-2- en-1-one | 601.0 | Column: Chiralcel OX (21 × 150 mm, 5 μm). Mobile phase: 45% MeOH Flowrate: 100 mL/min. |
| 3-091-1 | 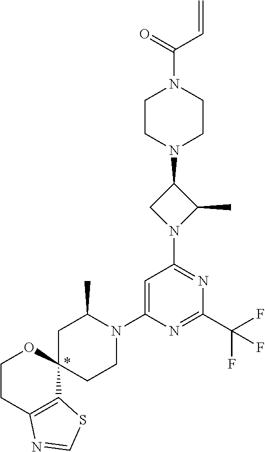 [SMILES: C=CC(=O)N1CCN([C@@H]2CN(c3cc(N4CC[C@]5(C[C@H]4C)OCCc4ncsc45)nc(C(F)(F)F)n3)[C@@H]2C)CC1] | Peak 1: 1-(4-((2R,3R)-2- methyl-1-(6-((2R.4R)-2- methyl-6′,7′- dihydrospiro[piperidine- 4,4′-pyrano[4,3-d]thiazol]- 1-yl)-2- (trifluoromethyl)pyrimidin- 4-yl)azetidin-3- yl)piperazin-1-yl)prop-2- en-1-one | 578.3 | Column: Daicel Chiralpak IG (30 × 250 mm, 10 μm). Mobile phase: 45% MeOH (0.1% NH₃) Flowrate: 75 mL/min. |
| 3-091-2 | 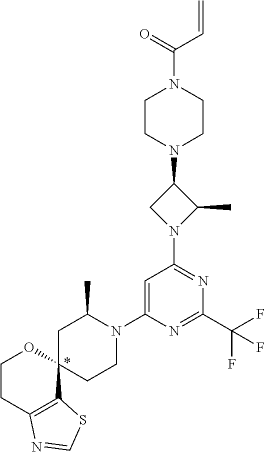 [SMILES: C=CC(=O)N1CCN([C@@H]2CN(c3cc(N4CC[C@@]5(C[C@H]4C)OCCc4ncsc45)nc(C(F)(F)F)n3)[C@@H]2C)CC1] | Peak 2: 1-(4-((2R,3R)-2- methyl-1-(6-((2S,4S)-2- methyl-6′,7′- dihydrospiro[piperidine- 4,4′-pyrano[4,3-d]thiazol]- 1-yl)-2- (trifluoromethyl)pyrimidin- 4-yl)azetidin-3- yl)piperazin-1-yl)prop-2- en-1-one | 578.3 | Column: Daicel Chiralpak IG (30 × 250 mm, 10 μm). Mobile phase: 45% MeOH (0.1% NH₃) Flowrate: 75 mL/min. |

Other compounds disclosed herein can be prepared by analogous methods to the general methods and example above.

### Section 3: Biochemical and Cellular Assays

Provided in this section is the biological evaluation of the specific examples provided herein.

### Example A. Coupled Nucleotide Exchange Assay

The KRAS^{G12C} coupled nucleotide exchange assay allows for the screening and profiling of KRAS^{G12C} antagonists/inhibitors by monitoring the binding of an effector protein (e.g., a Ras binding domain of Raf1, RBD-cRaf) to KRAS^{G12C}. Purified GDP-bound KRAS protein (aa 1-169), containing both G12C and C118A amino acid substitutions and an N-terminal His-tag, was pre-incubated in assay buffer (25 mM HEPES pH 7.4, 10 mM MgCl₂, 0.01% Triton X-100, either with or without 0.1% BSA) with serially diluted compound for 2 h. For all subsequent steps, BSA was omitted and DTT was added to the reaction buffer at a final concentration of 1 mM. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 5-30 min. The assay plates were then read on a plate reader measuring luminescence signal. Signal intensity of compound-containing wells were normalized to DMSO control, and data were analyzed using a 4-parameter logistic model to calculate IC₅₀ values.

### Example B. Cell Viability Assay

MIA PaCa-2 (human pancreatic carcinoma; ATCC CRL-1420) or A549 (human lung carcinoma; ATCC CCL-185) cells were cultured in RPMI 1640 medium containing 10% fetal bovine serum and 1×penicillin/streptomycin/L-glutamine. Cells were seeded in 384-well plates at a density of 1.67E+04 cells/mL and incubated at 37° C., 5% CO₂, overnight. Serially-diluted compound or DMSO was added to the cells, and plates were incubated at 37° C., 5% CO₂ for 72 h. Cell viability was measured using a CellTiter-Glo® Luminescent Cell Viability Assay kit (Promega) according to the manufacturer's protocol. The luminescence signal of treated samples was normalized to DMSO control, and data were analyzed using a 4-parameter logistic model to calculate IC₅₀ values.

The following data (Table 3) categorizes the IC₅₀ of each compound for inhibiting enzymatic activity of KRAS^{G12C} in the indicated cells.

TABLE 3

| Example | Coupled Exchange IC₅₀ | MIA PaCa-2, IC₅₀ | A549, IC₅₀ |
|---|---|---|---|
| # | (μM) | (μM) | (μM) |
| 3-001 | 0.376 | 0.665 | >5 |
| 3-002 | 0.315 | 0.625 | >5 |
| 3-003 | 0.512 | 0.832 | >5 |
| 3-004 | 0.154 | 0.527 | >5 |
| 3-005 | 0.184 | 0.626 | >5 |
| 3-006 | 2.24 | NT | NT |
| 3-007 | 6.57 | NT | NT |
| 3-008 | 0.275 | 0.625 | NT |
| 3-009 | 0.884 | NT | NT |
| 3-010 | 0.660 | NT | NT |
| 3-011 | 0.964 | NT | NT |
| 3-012 | 2.54 | NT | NT |
| 3-013 | 5.64 | NT | NT |
| 3-014 | 0.498 | 0.694 | >5 |
| 3-015 | 0.669 | 2.76 | >5 |
| 3-016 | 0.969 | >5 | >5 |
| 3-017 | 0.828 | 1.99 | >5 |
| 3-018 | 1.98 | NT | NT |
| 3-019 | 4.4 | NT | NT |
| 3-020 | 1.6 | NT | NT |
| 3-021 | 3.86 | NT | NT |
| 3-022 | 5.61 | NT | NT |
| 3-023 | 7.27 | NT | NT |
| 3-024 | 6.10 | NT | NT |
| 3-025 | 3.72 | NT | NT |
| 3-026 | 1.95 | NT | NT |
| 3-027 | 6.15 | NT | NT |
| 3-028 | 1.79 | 1.34 | >3.33 |
| 3-029 | 8.68 | NT | NT |
| 3-030 | 9.59 | NT | NT |
| 3-031 | 9.25 | NT | NT |
| 3-032 | 2.75 | NT | NT |
| 3-033 | 6.19 | NT | NT |
| 3-034 | 2.45 | NT | NT |
| 3-035 | 3.58 | NT | NT |
| 3-036 | 6.70 | NT | NT |
| 3-037 | 3.30 | NT | NT |
| 3-038 | 5.70 | NT | NT |
| 3-039 | 1.645 | 2.41 | >5 |
| 3-040 | 2.84 | NT | NT |
| 3-041 | >6.25 | NT | NT |
| 3-042 | 5.12 | NT | NT |
| 3-043 | 1.59 | 1.29 | >5 |
| 3-044 | 3.32 | NT | NT |
| 3-045 | 6.35 | NT | NT |
| 3-046 | 0.253 | 1.14 | >5 |
| 3-047 | 2.69 | NT | NT |
| 3-048 | 2.29 | NT | NT |
| 3-049 | 0.664 | 0.979 | >5 |
| 3-050 | 0.825 | NT | NT |
| 3-051 | 0.102 | 0.284 | >5 |
| 3-052 | 0.161 | 0.254 | 4.55 |
| 3-053 | 0.191 | 0.755 | >5 |
| 3-054-1 | 0.128 | 0.603 | >5 |
| 3-054-2 | 0.381 | 1.49 | >5 |
| 3-055 | 1.32 | NT | NT |
| 3-057 | 0.2885 | 0.272 | >5 |
| 3-058 | 0.9195 | >5 | >5 |
| 3-059 | 0.2145 | 0.582 | >5 |
| 3-060 | 0.406 | 1.09 | >5 |
| 3-061 | 0.184 | 0.936 | >5 |
| 3-062-1 | 4.66 | NT | NT |
| 3-062-2 | 6.2 | NT | NT |
| 3-063 | 8.24 | NT | NT |
| 3-064 | 5.70 | NT | NT |
| 3-065 | 0.752 | NT | NT |
| 3-066 | 0.55 | NT | NT |
| 3-067-1 | 0.527 | 1.31 | >5 |
| 3-067-2 | 0.867 | NT | NT |
| 3-068-1 | 1.28 | NT | NT |
| 3-068-2 | 1.54 | NT | NT |
| 3-069 | 0.830 | NT | NT |
| 3-070 | 1.40 | NT | NT |
| 3-071 | 2.99 | NT | NT |
| 3-072 | 4.90 | NT | NT |
| 3-073 | 4.3 | NT | NT |
| 3-074 | 0.423 | 1.16 | >5 |
| 3-075 | 0.704 | 2.8 | >5 |
| 3-076 | 0.072 | 0.214 | >5 |
| 3-077 | 0.021 | 0.182 | >5 |
| 3-078 | 0.089 | 0.387 | >5 |
| 3-079 | 0.081 | 0.18 | >5 |
| 3-080 | 0.353 | NT | NT |
| 3-081 | 0.063 | 0.131 | >5 |
| 3-082 | 0.069 | 0.145 | >5 |
| 3-083 | 0.075 | 0.171 | >5 |
| 3-084 | 0.176 | 0.172 | >5 |
| 3-085 | 0.052 | 0.180 | >5 |
| 3-086 | 2.91 | NT | NT |
| 3-087-1 | 0.048 | 0.078 | >5 |
| 3-087-2 | 0.087 | 0.142 | >5 |
| 3-088-1 | 0.106 | 0.189 | >5 |
| 3-088-2 | 0.072 | 0.128 | >5 |
| 3-089-1 | 0.042 | 0.102 | >5 |
| 3-090-1 | 0.145 | 0.193 | >5 |
| 3-091-1 | 0.054 | 0.331 | >5 |
| 3-091-2 | 0.883 | NT | NT |
| 3-092 | 2.40 | NT | NT |
| 3-093 | 0.107 | 0.271 | >5.0 |
| 3-094 | 0.052 | 0.175 | >5.0 |
| 3-095 | 0.031 | 0.159 | >5.0 |
| 3-096 | 0.069 | 0.267 | >5.0 |
| 3-097 | 0.085 | 0.226 | >5.0 |
| 3-098 | 0.131 | 0.224 | >5.0 |
| 3-099 | 0.041 | 0.165 | >5.0 |
| 3-100 | 0.040 | 0.155 | >5.0 |
| 3-101 | 0.070 | 0.259 | >5.0 |
| 3-102 | 0.023 | 0.077 | >5.0 |
| 3-103 | 0.036 | 0.171 | >5.0 |
| 3-104 | 0.021 | 0.081 | >5.0 |

NT = not tested

The results presented in Table 3 have been generated with the in vitro assays described above. These assays may be used to test any of the compounds described herein to assess and characterize a compound's biological activity. In view of the disclosure provided herein, compounds not specifically tested would be expected to have similar results.

Compounds showing activity in the coupled exchange assay are useful in the methods provided herein (see Section “METHODS OF USE”). See, e.g., Lanman et al., 2020; Hong et al., 2020. The inhibitory effect on tumor growth of the compounds provided herein can be shown, for example, using the following animal model.

Tumor cells are cultured, harvested and implanted subcutaneously into the right flank of female athymic nude mice. When tumors reach about 200 mm³, mice are randomized into treatment groups (n=10/group) and treatment is initiated (on days indicated on graphs). Tumor sizes and body weights are measured 2 to 3 times per week. Tumor volume is measured by digital calipers, calculated as L×W×H and expressed in mm³. Statistical significance of observed differences between growth curves can be evaluated by repeated measures analysis of covariance (RMANOVA) of the log transformed tumor volume data with Dunnett adjusted multiple comparisons comparing the control group to the treatment group. For combination studies, RMANOVA can be run with the combination group compared one to one with each single agent treatment group.

The foregoing description is provided for clearness of understanding only. No unnecessary limitations should be understood therefrom, as modifications within the scope of the invention may be apparent to those having ordinary skill in the art.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

All references, for example, a scientific publication, a patent, or a patent application publication, cited in this disclosure are incorporated herein by reference in their entirety and for all purposes to the same extent as if each reference was specifically and individually indicated to be incorporated by reference in its entirety for all purposes. In case of conflict between the present disclosure and incorporated references, the present disclosure should control.

What is claimed is:

1. A compound of Formula (II): 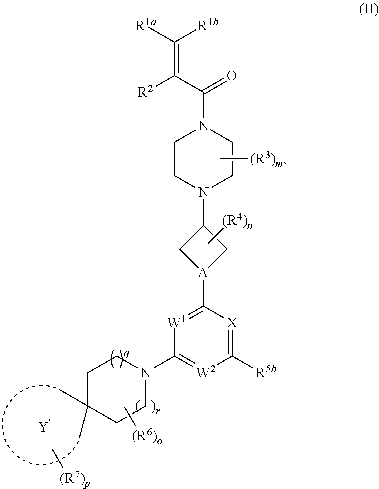
or a pharmaceutically acceptable salt thereof,
wherein:
m is 0, 1, 2, 3, or 4;
n is 0, 1, or 2;
o is 0, 1, 2, 3, or 4;
p is 0, 1, 2, 3, 4, or 5;
q is 0, 1, or 2;
r is 0 or 1;
A is N, CH, C-halo, C—CN, C—C₁₋₃alkyl, C—C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃haloalkyl, C—C₀₋₃alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkylencOH, or C₀₋₃alkyleneC₁₋₄alkoxy;
X is N or C—R^{5a};
Y′ is C₃₋₇cycloalkyl, C₄₋₇cycloalkenyl, heterocycloalkyl having 3 to 7 total ring atoms and 1-3 heteroatoms independently selected from N, O, and S, or heterocycloalkenyl having 5 to 7 total ring atoms and 1-3 heteroatoms independently selected from N, O, and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form 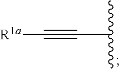
each R³ independently is C₁₋₃alkyl, C₁₋₃haloalkyl, 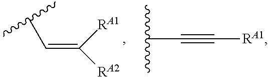
C₀₋₃alkyleneCN, C₀₋₃alkyleneOH, C₀₋₃alkylene-C₁₋₃alkoxy, oxo, spiro-C₃₋₇cycloalkyl, spiro-C₄₋₇cycloalkenyl, spiro-heterocycloalkyl having 3-7 total ring atoms and 1 or 2 heteroatoms independently selected from N, O, and S, or spiro-heterocycloalkenyl having 4-7 total ring atoms and 1 or 2 heteroatoms independently selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms independently selected from N, O, and S, or fused-heterocycloalkenyl having 4-7 total ring atoms and 1 or 2 heteroatoms independently selected from N, O, and S;
each of R^{A1} and R^{A2} independently is H, C₁₋₃alkyl, C₁₋₃haloalkyl, or C₃₋₅cycloalkyl;
each R⁴ independently is C₁₋₃alkyl, C₁₋₃haloalkyl, C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 heteroatoms independently selected from N, O and S;
R^{5a} is H, CN, halo, C₁₋₃alkyl, C₁₋₃haloalkyl, C₂₋₆alkenyl, C₂₋₆alkynyl, C₀₋₃alkyleneOH, C₀₋₃alkylene-C₁₋₃alkoxy, or C₃₋₅cycloalkyl;
R^{5b} is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms independently selected from N, O, and S, heterocycloalkenyl having 5-7 total ring atoms and 1-3 heteroatoms independently selected from N, O, and S, or R^{5a} and R^{5b}, together with the atoms to which they are attached, form C₃₋₇cycloalkyl;
wherein each of the foregoing C₁₋₆alkyl, C₂₋₄alkenyl, C₂₋₄alkynyl, C₃₋₇cycloalkyl, C₅₋₇cycloalkenyl, heterocycloalkyl, and heterocycloalkenyl independently is unsubstituted or substituted with 1-3 substituents, and each substituent independently is C₁₋₃haloalkyl, C₀₋₆alkylene-OH, C₀₋₃alkylene-C₁₋₃alkoxy, C₃₋₇cycloalkyl, C₅₋₇cycloalkenyl, heterocycloalkyl having 3-7 total ring atoms and 1-3 heteroatoms independently selected from N, O, and S, heterocycloalkenyl having 4-7 total ring atoms and 1-3 heteroatoms independently selected from N, O, and S, or phenyl;
each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-Ca-cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₃alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

2. The compound or salt of claim 1, wherein at least one of R^{1a}, R^{1b}, and R² is H or D.

3. The compound or salt of claim 1 or 2, wherein each of R^{1a}, R^{1b}, and R² independently is H or D.

4. The compound or salt of claim 1 or 2, wherein at least one of R^{1a}, R^{1b}, and R² is halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms independently selected from N, O, and S, or R^{1b} and R², together with the carbon atoms to which they are attached, form 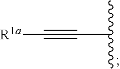
wherein each R^{N1} independently is H or CH₃.

5. The compound or salt of claim 4, wherein at least one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₂, CH₂N(CH₃)₂, aziridin-1-yl-methyl, azetidin-1-yl-methyl, pyrrolidine-1-yl-methyl, piperidin-1-yl-methyl, or morpholin-1-yl-methyl.

6. The compound or salt of claim 1 or 2, wherein 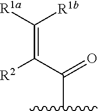
is

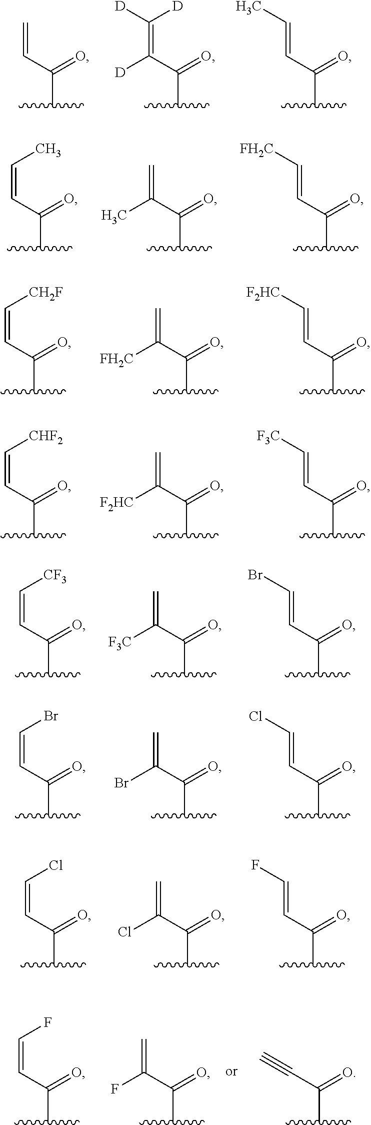

7. The compound or salt of claim 6, wherein 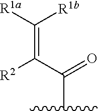
is

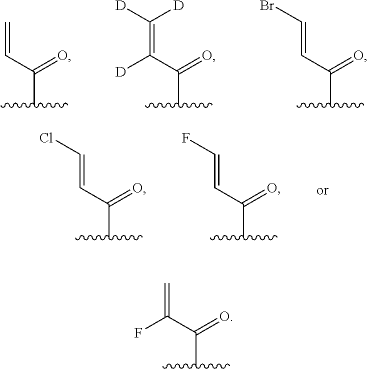

8. The compound or salt of claim 7, wherein 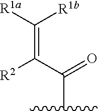
is

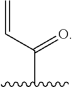 [SMILES: *C(=O)C=C]

9. The compound or salt of any one of claims 1-8, wherein m is 0 or 1.

10. The compound or salt of any one of claims 1-8, wherein in is 2, 3, or 4.

11. The compound or salt of any one of claims 1-10, wherein at least one R³ is C₁₋₃alkyl or C₁₋₃haloalkyl.

12. The compound or salt of claim 11, wherein at least one R³ is CH₃, CH CH₃, CF₃, CHF₂, or CH₂F.

13. The compound or salt of any one of claims 1-10, wherein at least one R³ is 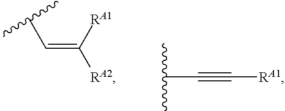
C₀₋₃alkyleneCN, C₀₋₃alkyleneOH, C₀₋₃alkylene-C₁₋₃alkoxy, oxo, spiro-C₃₋₇cycloalkyl, spiro-heterocycloalkyl having 3-7 total ring atoms and 1 or 2 heteroatoms independently selected from N, O and S, spiro-C₄₋₇cycloalkenyl, spiro-heterocycloalkenyl having 4-7 total ring atoms and 1 or 2 heteroatoms independently selected from N, O and S; two vicinal R³, together with the atoms to which they are attached, form fused-C₃₋₇cycloalkyl, fused-heterocycloalkyl having 3-7 total ring atoms and 1 or 2 heteroatoms independently selected from N, O and S, fused-C₄₋₇cycloalkenyl, or fused-heterocycloalkenyl having 4-7 total ring atoms and 1 or 2 heteroatoms independently selected from N, O and S; wherein each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl.

14. The compound or salt of claim 13, wherein at least one R³ is 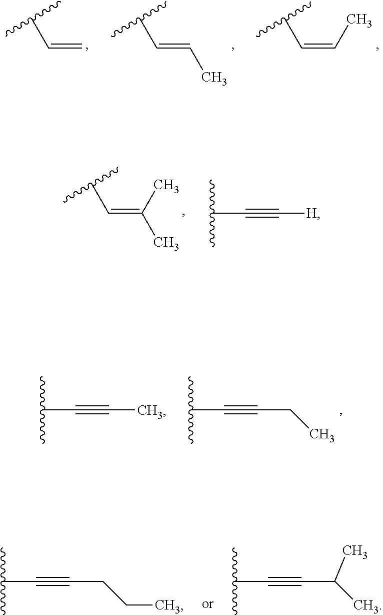
CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, spiro-tetrahydrofuranyl, or two vicinal R³, together with the atoms to which they are attached, form fused-cyclopropyl or fused-cyclobutyl.

15. The compound or salt of any one of claims 1-10, wherein each R³ independently is CH₃, CH₂CH₃, CF₃, CHF₂, CH₂F, 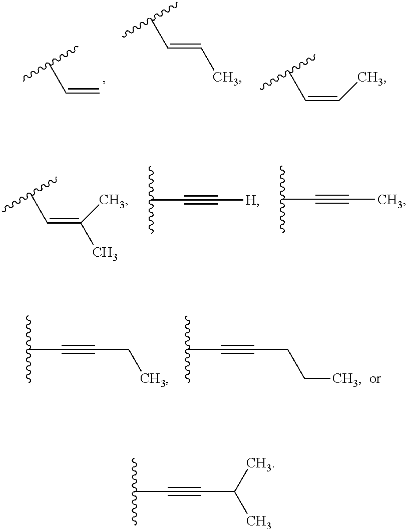
CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl.

16. The compound or salt of any one of claims 1-8, wherein m is 0; or m is 1 and R³ is CH₃, CH₂F, CHF₂, CF₃, CN, CH₂CN, CH₂OH, CH₂OCH₃, or spiro-oxetanyl.

17. The compound or salt of claim 16, wherein m is 0; or m is 1 and R³ is CH₃.

18. The compound or salt of any one of claims 1-8, wherein 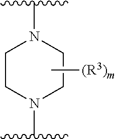
is

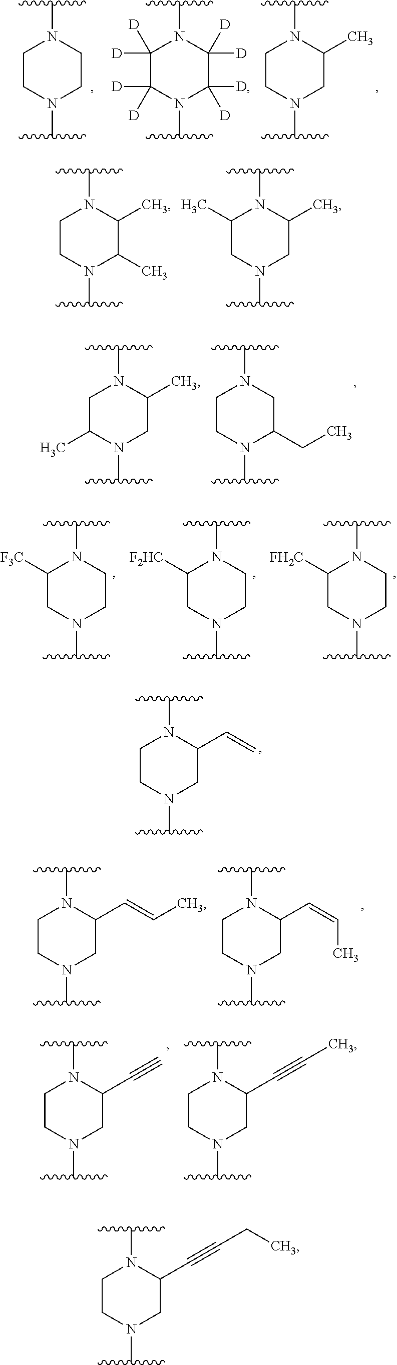

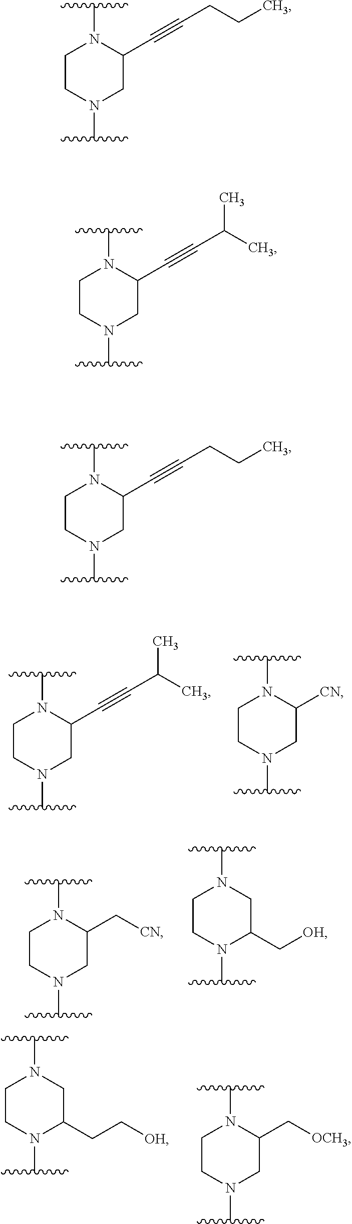

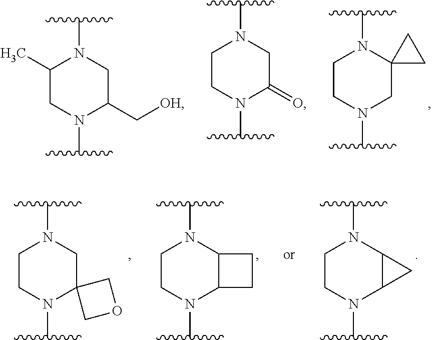

19. The compound or salt of claim 18, wherein 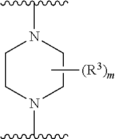
is

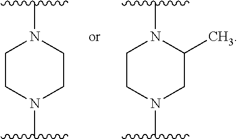 [SMILES: *N1CCN(*)CC1 ; *N1CCN(*)C(C)C1]

20. The compound or salt of any one of claims 1-19, wherein A is N.

21. The compound or salt of any one of claims 1-19, wherein A is CH, C-halo, C—CN, C—C₁₋₃alkyl, C—C₁₋₃haloalkyl, C—C₀₋₃alkyleneOH, or C—C₀₋₃alkylene-C₁₋₄alkoxy.

22. The compound or salt of claim 21, wherein A is CH, C—F, C—Cl, C—CN, C—CH₃, C—CH₂F, C—CHF₂, C—CF₃, C—OH, C—CH—OH, C—OCH₃, or C—CH₂OCH₃.

23. The compound or salt of any one of claims 1-22, wherein n is 0 or 1.

24. The compound or salt of any one of claims 1-23, wherein at least one R⁴ is C₁₋₃alkyl or C₁₋₃haloalkyl.

25. The compound or salt of claim 24, wherein at least one R⁴ is CH₃, CH₂CH₃, CH₂CH₂CH₃, CH(CH₃)₂, CH₂F, CHF₂, or CF₃.

26. The compound or salt of any one of claims 1-23, wherein at least one R⁴ is C₀₋₃alkyleneCN, C₁₋₃alkyleneOH, C₁₋₃alkylene-C₁₋₃alkoxy, oxo, spiro-C₃₋₇cycloalkyl, or spiro-heterocycloalkyl having 4-7 total ring atoms and 1 or 2 heteroatoms independently selected from N, O and S.

27. The compound or salt of claim 26, wherein at least one R⁴ is CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl.

28. The compound or salt of any one of claims 1-23, wherein each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl.

29. The compound or salt of claim 28, wherein 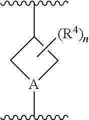
is

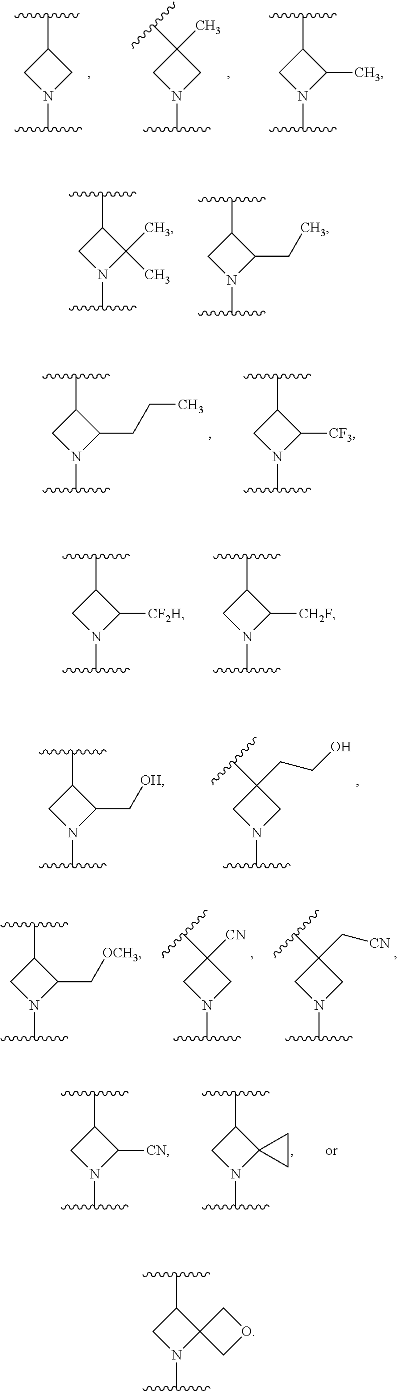

30. The compound or salt of claim 29, wherein 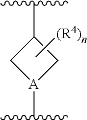
is

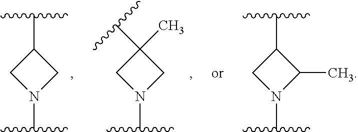

31. The compound or salt of any one of claims 1-30, wherein W¹ is N, CH, C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), or C—CCH, C—OH, C—CH₂OH, C—OCH₃, or C—CH₂OCH₃.

32. The compound or salt of claim 31, wherein W¹ is CH.

33. The compound or salt of any one of claims 1-32, wherein W² is N, CH, C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), or C—CCH, C—OH, C—CH₂OH, C—OCH₃, or C—CH₂OCH₃.

34. The compound or salt of claim 33, wherein W² is N.

35. The compound or salt of any one of claims 1-30, wherein W¹ is CH and W² is N.

36. The compound or salt of any one of claims 1-35, wherein X is N.

37. The compound or salt of any one of claims 1-35, wherein X is C—R^{5a} and R^{5a} is H, CN, Br, Cl, F, CH₃, CH₂CH₃, CF₃, CHF₂, CH₂F, 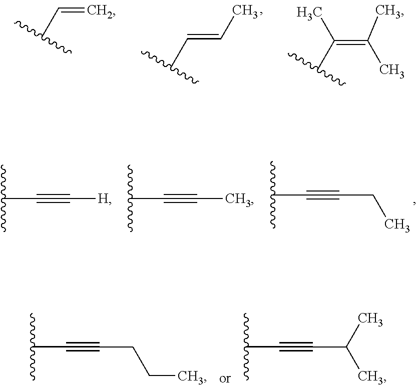
OH, CH₂OH, OCH₃, CH₂OCH₃, or

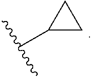 [SMILES: *C1CC1]

38. The compound or salt of claim 37, wherein R^{5a} is CN or CH₃.

39. The compound or salt of any one of claims 1-35, wherein X is N, C—CH₃, or C—CN.

40. The compound or salt of any one of claims 1-30, wherein W¹ is CH; W² is N; and X is N, C—CH₃, or C—CN.

41. The compound or salt of any one of claims 1-40, wherein R^{5b} is Br, Cl, F, CF₃, CF₂H, CFH₂, CF₂CH₃, OCH₃, SCH₃, CH₃, CH₂CH₃, CH₂CH₂CH₃, CH(CH₃)₂,

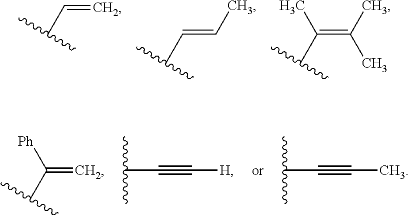

42. The compound or salt of claim 41, wherein R^{5b} is CF₃ or CF₂H.

43. The compound or salt of any one of claims 1-30, wherein 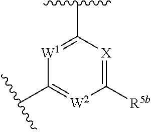
is 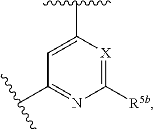
X is N, C—CH₃, or C—CN; and R^{b} is CF₃ or CF₂H.

44. The compound or salt of any one of claims 1-30, wherein 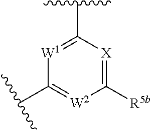
is

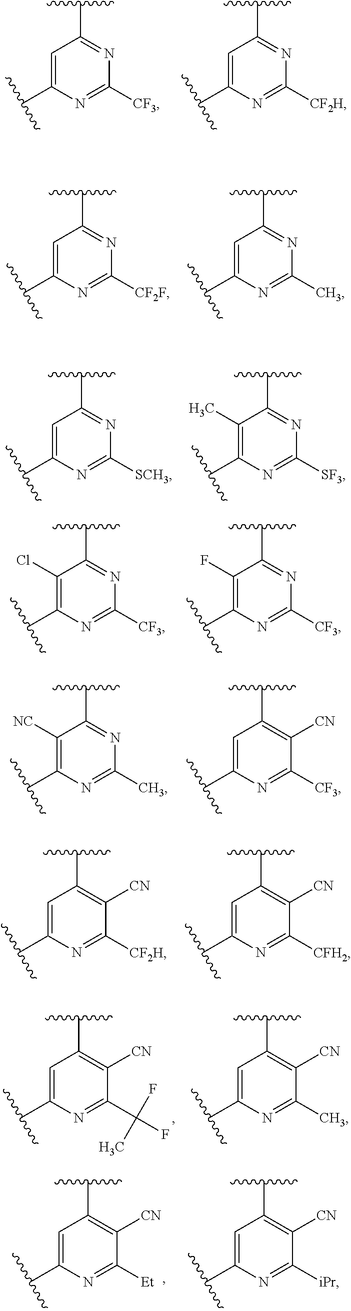

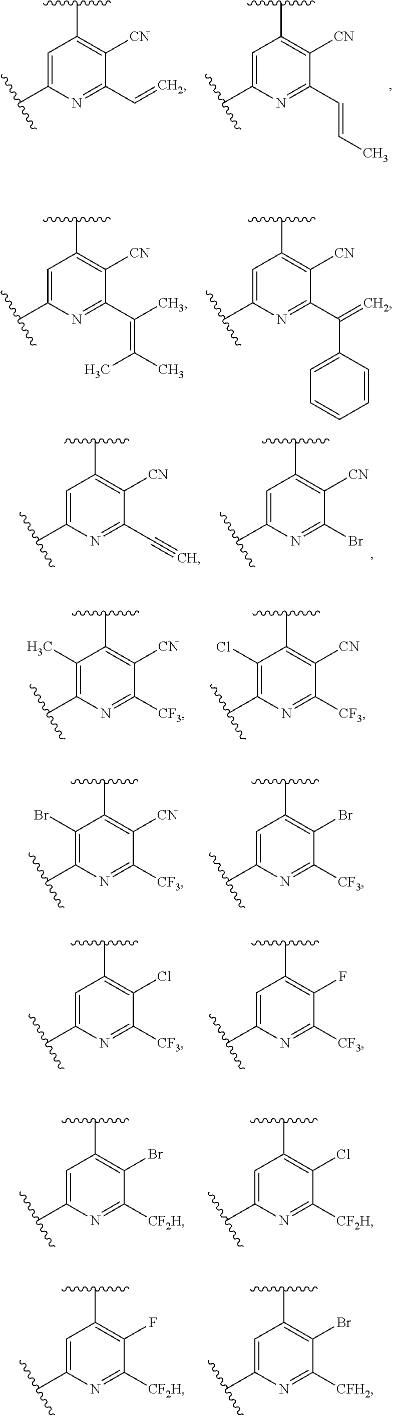

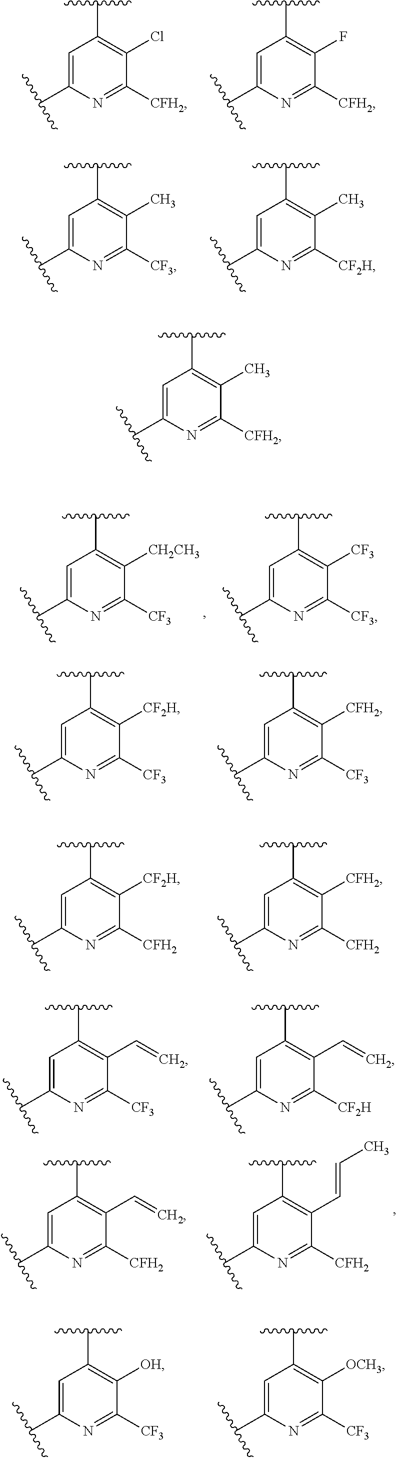

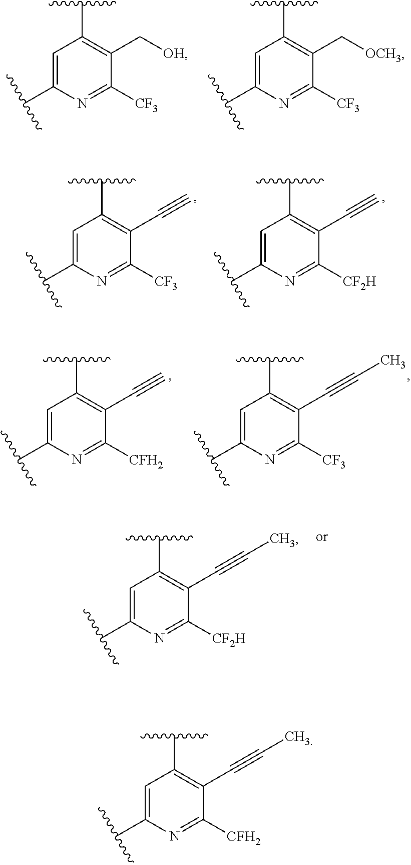

45. The compound or salt of claim 44, wherein 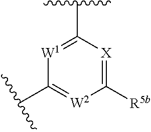
is

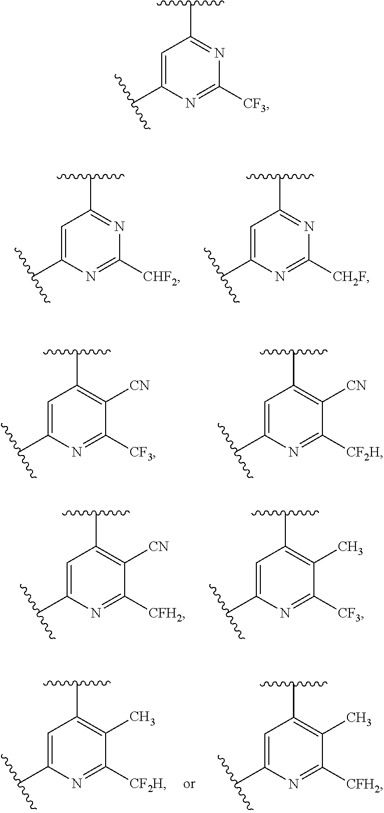

46. The compound or salt of any one of claims 1-45, wherein q is 0.

47. The compound or salt of any one of claims 1-45, wherein q is 1.

48. The compound or salt of any one of claims 1-45, wherein q is 2.

49. The compound or salt of any one of claims 1-45, wherein r is 0.

50. The compound or salt of any one of claims 1-45, wherein r is 1.

51. The compound or salt of any one of claim 1-45, wherein 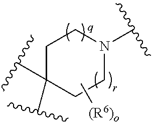
is

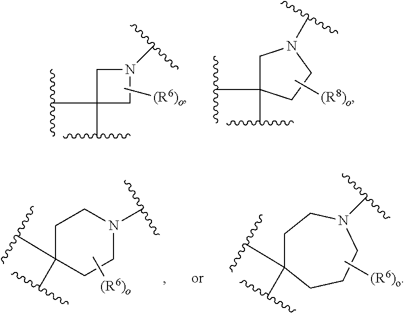

52. The compound of any one of claims 1-51, wherein o is 0, 1, or 2.

53. The compound of any one of claims 1-51, wherein o is 3 or 4.

54. The compound of any one of claims 1-53, wherein at least one R⁶ is Br, Cl, F, CN, CH₃, CH₂F, CHF₂, CF₃, OH, CH₂OH, OCH₃, OCD₃, CH₂OCH₃, CH₂N(CH₃)₂, oxo, ═CH₂, cyclopropyl, cyclobutyl, oxetanyl, tetrahydrofuranyl, spiro-cyclopropyl, spiro-cyclobutyl, spiro-oxetanyl, or spiro-tetrahydrofuranyl; or two vicinal R⁶, together with the atoms to which they are attached, form fused-cyclopropyl, fused-cyclobutyl, or fused-cyclopentyl; or two non-neighboring R⁶ join together to form —CH₂—, —CH₂CH₂—, —CH₂CH₂CH₂—, —CH₂—CH═CH— or —CH₂OCH₂—; wherein each of the cyclopropyl, cyclobutyl, oxetanyl, tetrahydrofuranyl, spiro-cyclopropyl, spiro-cyclobutyl, spiro-oxetanyl, spiro-tetrahydrofuranyl, fused-cyclopropyl, fused-cyclobutyl, and fused-cyclopentyl independently is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

55. The compound or salt of claim 54, wherein each substituent independently is F, Cl, OH, OCH₃, OCH CH₃, or CN.

56. The compound or salt of 54 or 55, wherein at least one R⁶ is Br, Cl, F, CN, CH₃, CH₂F, CHF₂, CF₃, OH, CH₂OH, OCH₃, OCD₃, CH₂OCH₃, CH₂N(CH₃)₂, oxo, ═CH₂, cyclopropyl, or two vicinal R⁶, together with the atoms to which they are attached, form fused-cyclopropyl, fused-cyclobutyl, or fused-cyclopentyl, or two non-neighboring R⁶ join together to form —CH₂—, —CH₂CH₂—, —CH₂CH₂CH₂—, —CH₂—CH═CH— or —CH₂OCH₂—; wherein each of the foregoing cycloalkyl groups is unsubstituted or substituted with 1-4 substituents, and each substituent independently is F, Cl, OH, OCH₃, OCH₂CH₃, or CN.

57. The compound of salt of any one of claims 1-51, wherein o is 0; or o is 1 and R⁶ is H, CH₃, CH₂OH, or two non-neighboring R⁶ join together to form —CH₂CH₂—.

58. The compound or salt of any one of claims 1-51, wherein 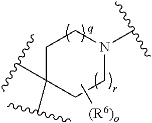
is

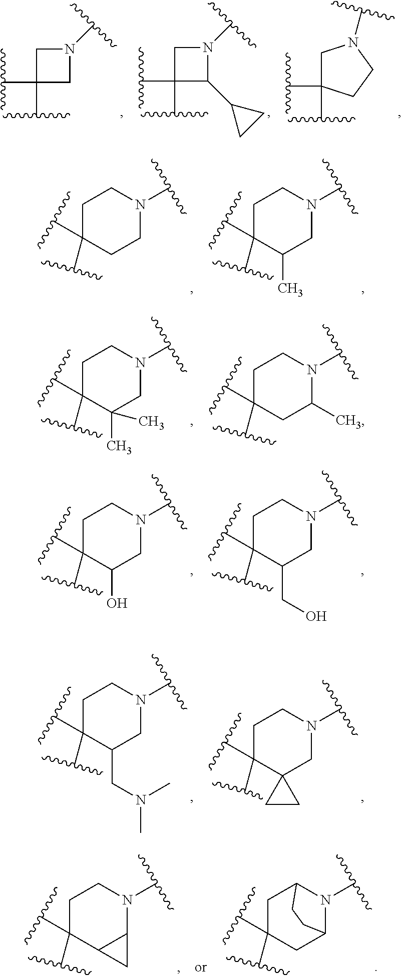

59. The compound or salt of claim 58, wherein 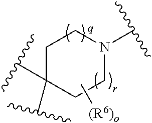
is 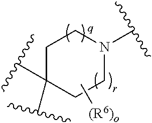
is

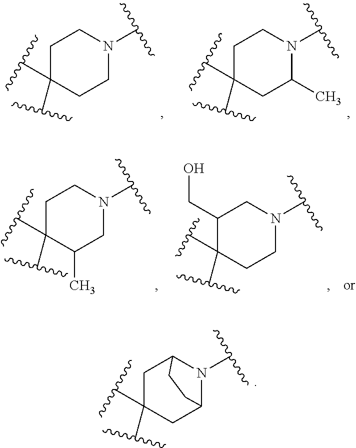

60. The compound or salt of any one of claims 1-30, wherein the compound is a compound of Formula (IIA), Formula (IIB), or Formula (IIC): 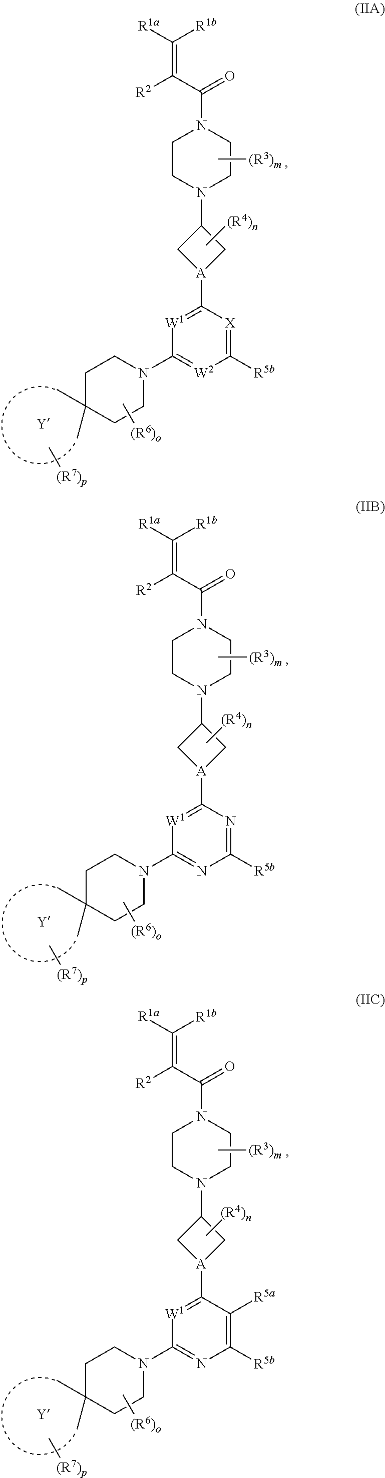
or a pharmaceutically acceptable salt of any of the foregoing.

61. The compound or salt of any one of claims 1-60, wherein Y′ is cyclopropyl, cyclobutyl, cyclopentyl, cyclohexyl, cyclopentenyl, cyclohexenyl, aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, dioxolanyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothiopyranyl, dithianyl, morpholinyl, thiomorpholinyl, dihydropyrrolyl, dihydrofuranyl, dihydrothiopheneyl, dihydroisoxazolyl, tetrahydropyridinyl, dihydropyranyl, or dihydrothipyranyl.

62. The compound or salt of claim 61, wherein Y′ is cyclopropyl, cyclobutyl, cyclopentyl, cyclopentenyl, oxetanyl, pyrrolidinyl, tetrahydrofuranyl, dioxolanyl, oxazolidinyl, isoxazolidinyl, tetrahydropyranyl, dihydropyrrolyl, dihydrofuranyl or dihydropyranyl.

63. The compound or salt of any one of claims 1-62, wherein p is 0 or 1.

64. The compound or salt of any one of claims 1-62, wherein p is 2, 3, or 4.

65. The compound or salt of any one of claims 1-64, wherein each R⁷ independently is F, Cl, Br, CN, CH₂CN, CH₃, CH₂CH₃, CH₂F, CHF₂, CF₃, OH, OCH₃, OCH₂CH₃, CH₂OH, CH₂OCH₃, CH₂CH₂OCH₃, C(═O)OCH₃, oxo, ═CH—, cyclopropyl, 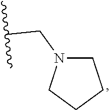 [SMILES: *CN1CCCC1]
oxetanyl, spiro-cyclopropyl, spiro-cyclobutyl, spiro-oxetanyl, spiro-tetrahydrofuranyl, spiro-tetrahydropyranyl; or two vicinal R⁷, together with the atoms to which they are attached, form fused-cyclopropyl, fused-cyclobutyl, fused-oxetanyl, fused-tetrahydrofuranyl, fused-phenyl, fused-thiophenyl, fused-pyrazolyl, fused-thiazolyl, fused-pyridyl, fused-pyridazinyl, fused-pyrazinyl, fused-pyrimidinyl; wherein each of the phenyl, pyrazolyl, pyridyl, pyridazinyl, and pyrimidinyl is unsubstituted or substituted with 1-3 substituents, and each substituent independently is halo, CN, oxo, or C₁₋₃alkyl.

66. The compound or salt of claim 65, wherein each R⁷ independently is F, Cl, Br, CN, CH₃, CH₂CH₃, CH₂F, OH, CH₂OH, CH₂OCH₃, CH₂CH₂OCH₃, oxo, ═CH₂, 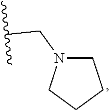 [SMILES: *CN1CCCC1]
oxetanyl, spiro-cyclopropyl, spiro-cyclobutyl, spiro-oxetanyl, or two vicinal R⁷, together with the atoms to which they are attached, form fused-cyclopropyl, fused-phenyl, fused-thiophenyl, fused-pyrazolyl, fused-thiazolyl, fused-pyridyl; wherein each of the phenyl, pyrazolyl, and pyridyl is unsubstituted or substituted with CH₃.

67. The compound or salt of any one of claims 1-59, wherein 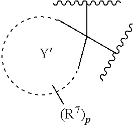
is

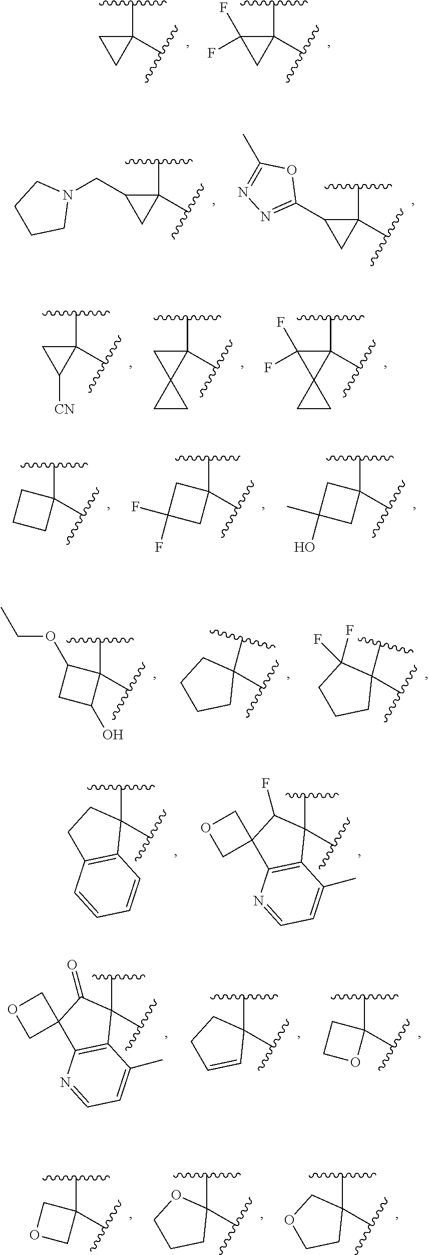

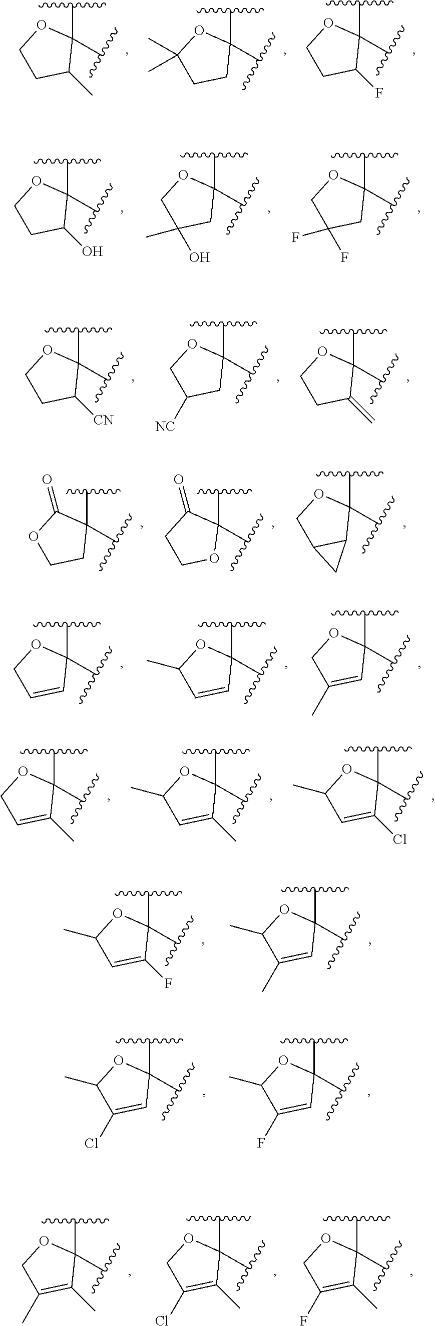

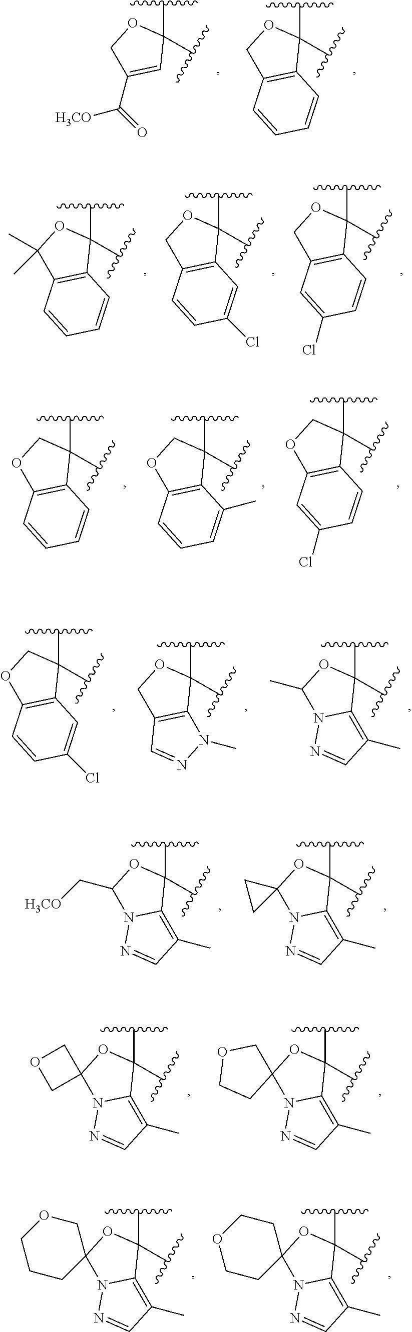

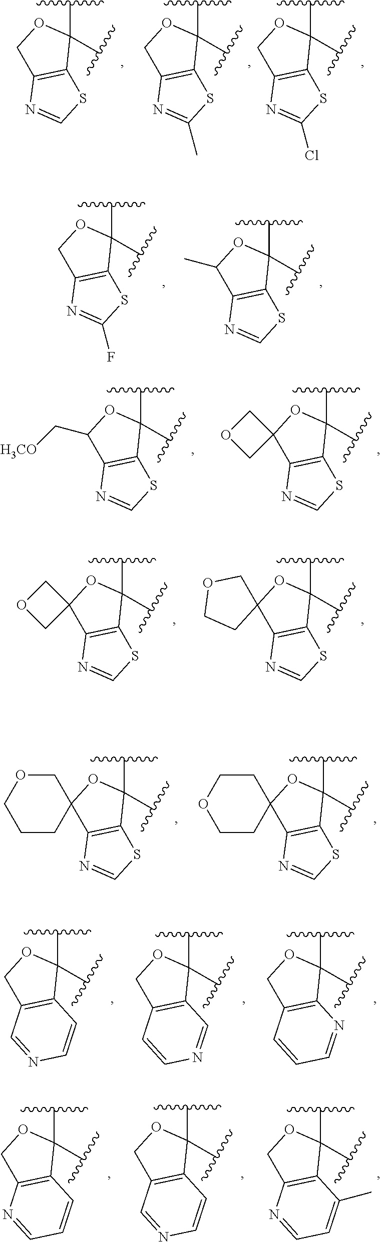

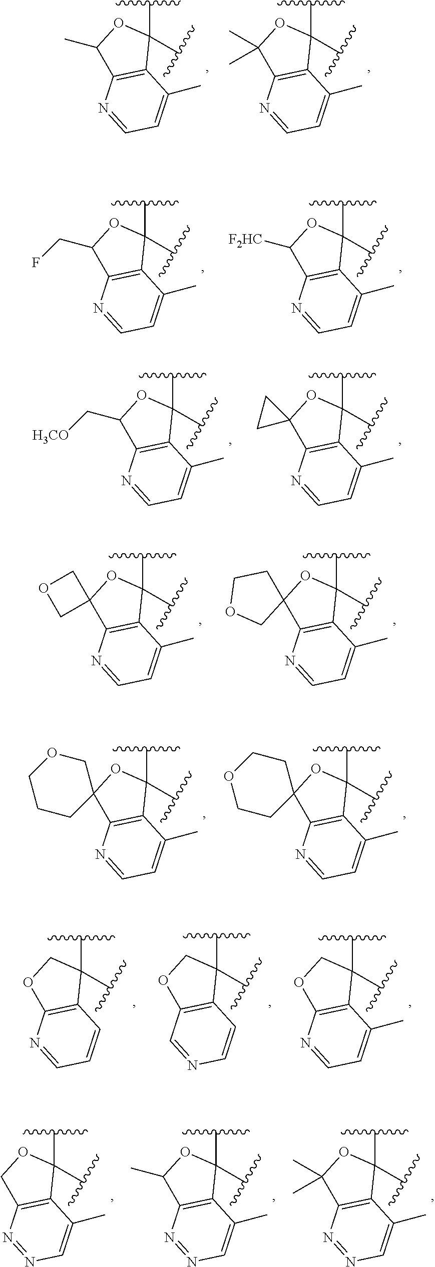

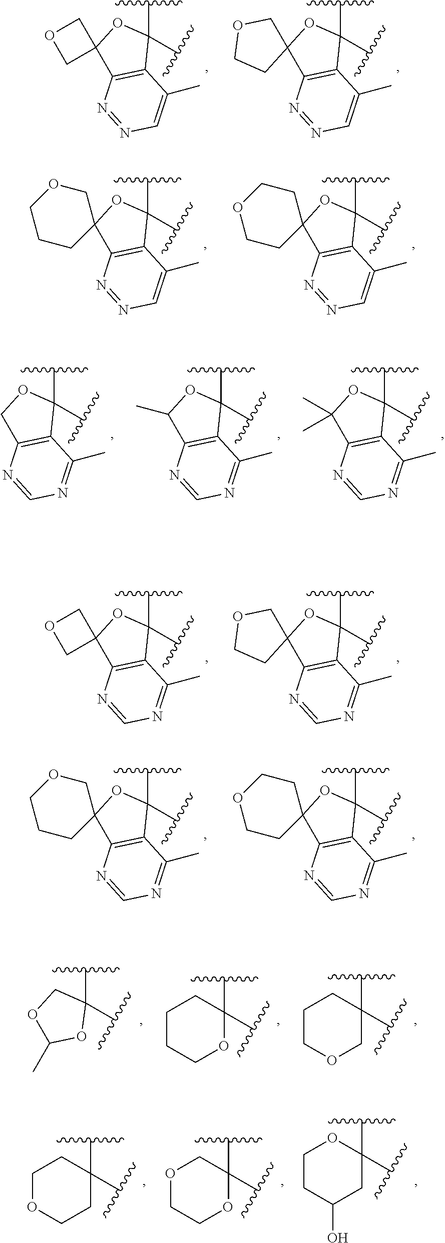

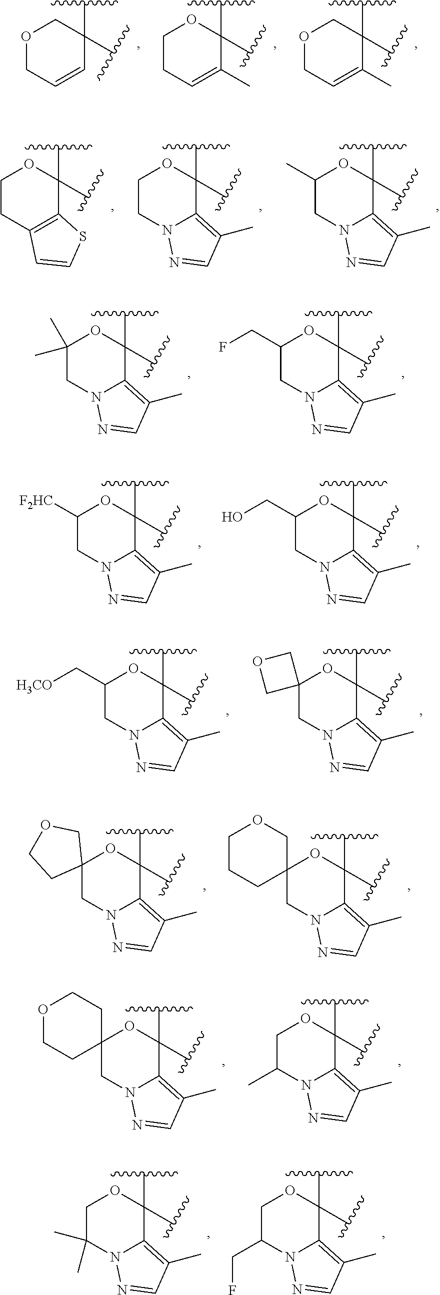

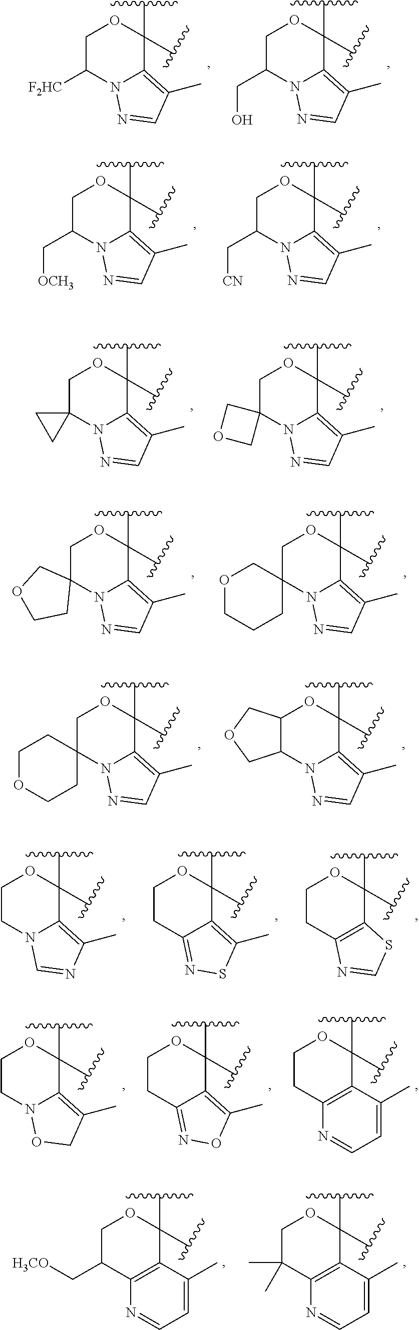

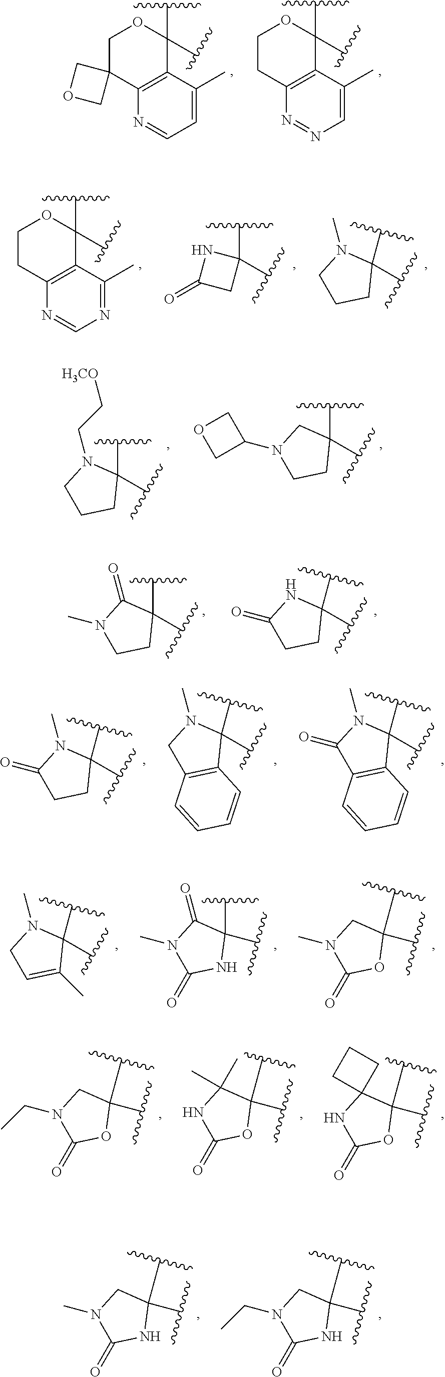

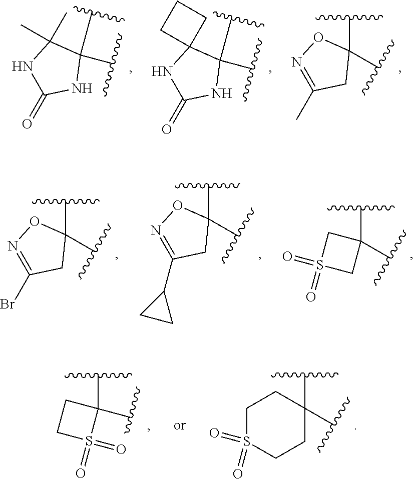

68. The compound or salt of any one of claims 1-59, wherein 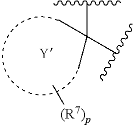
is

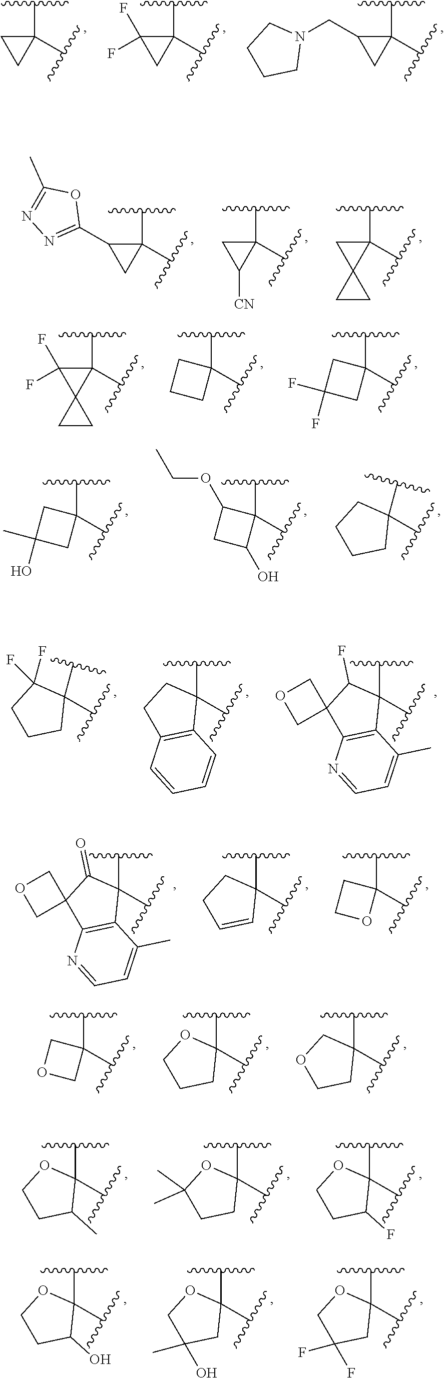

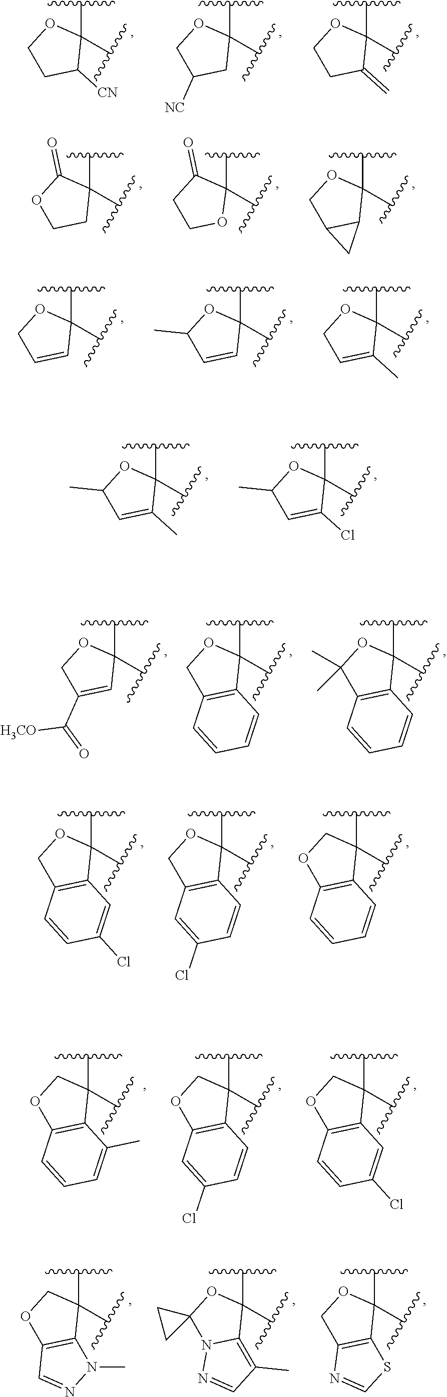

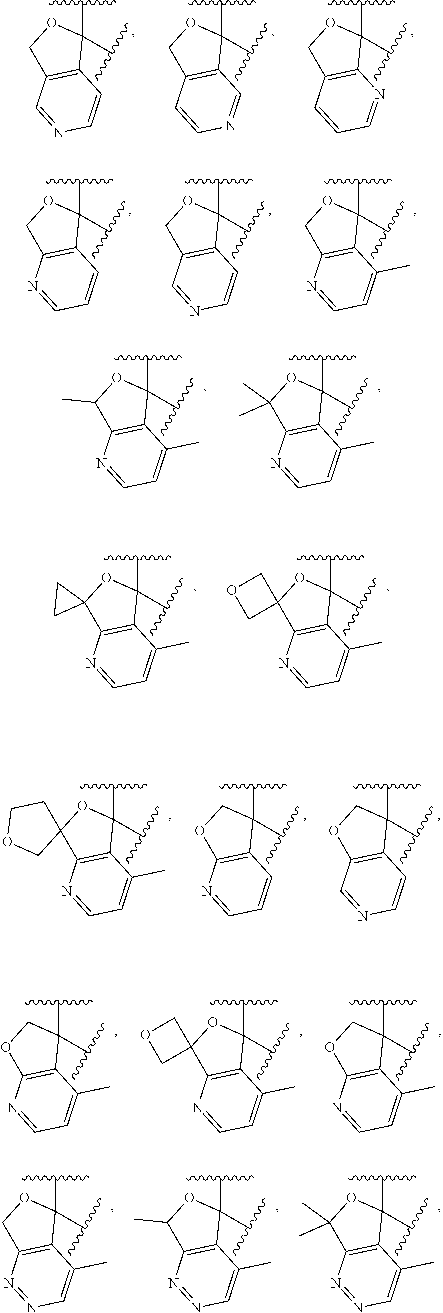

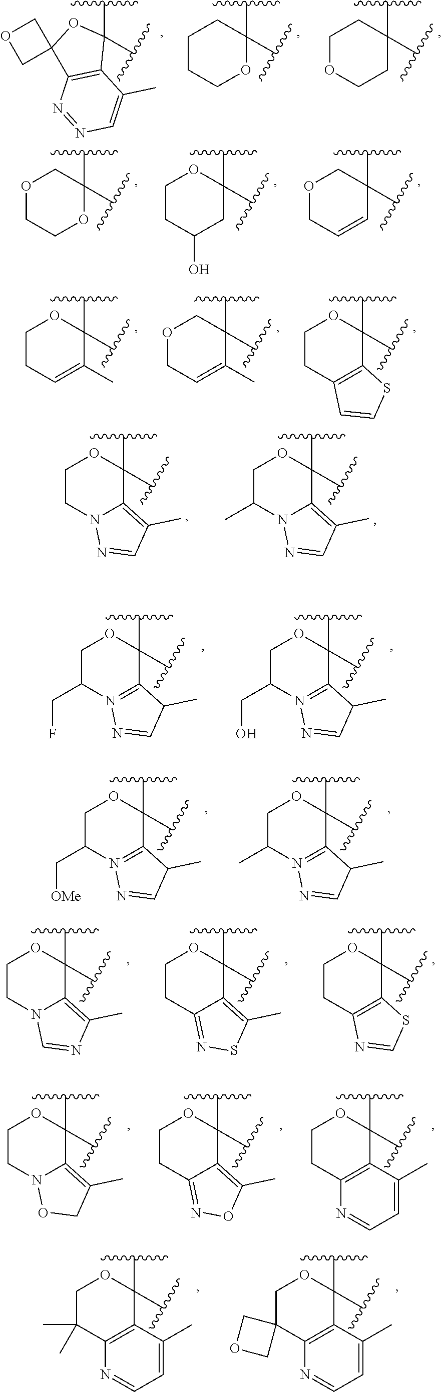

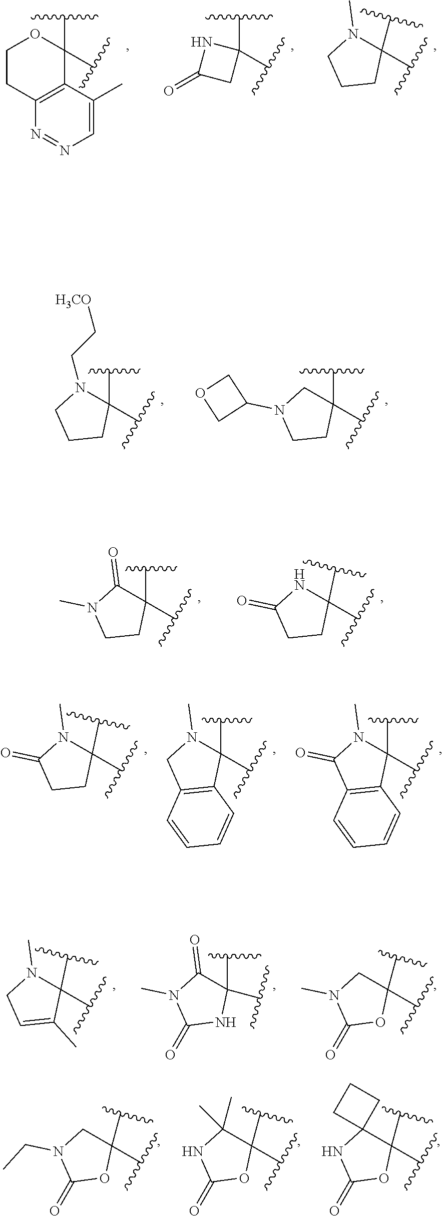

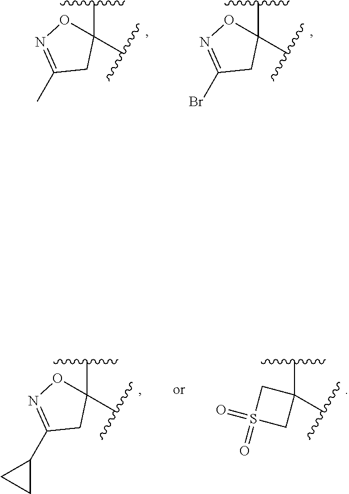

69. The compound or salt of claim 68, wherein 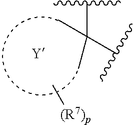
is

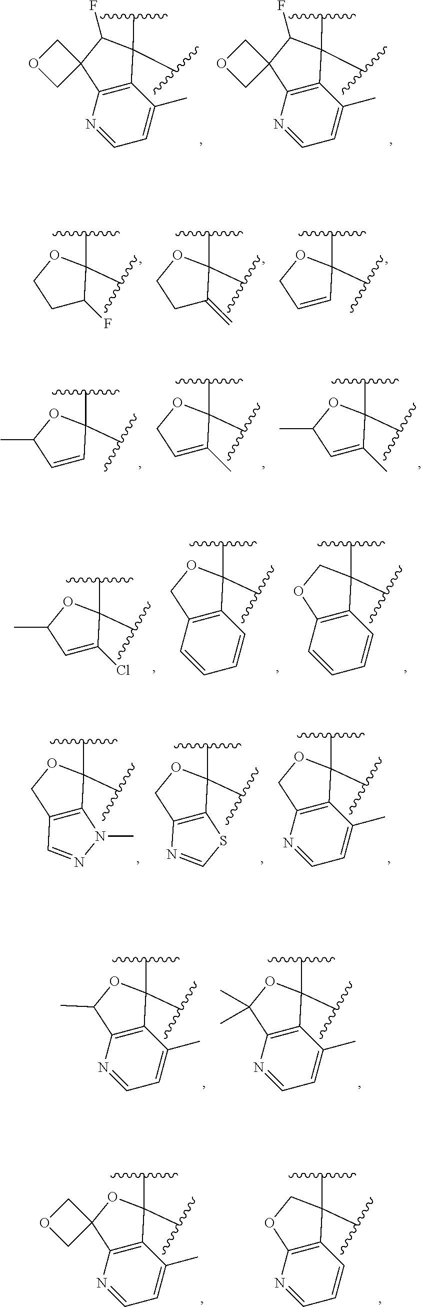

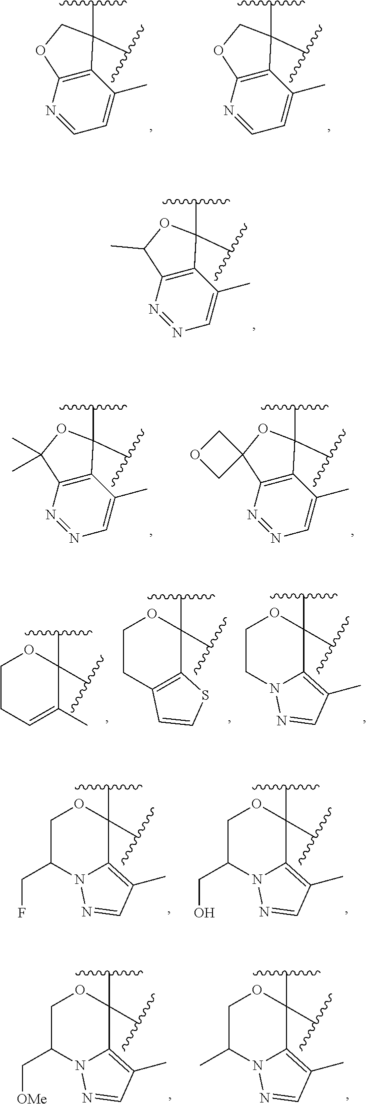

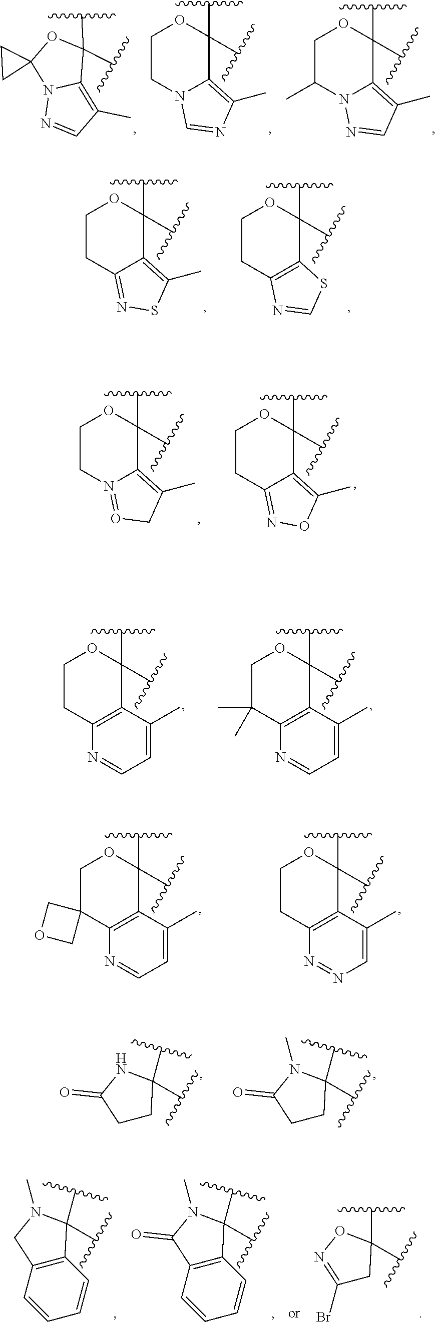

70. The compound or salt of claim 1, wherein: 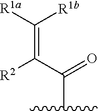
is 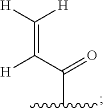 [SMILES: *C(=O)C=C] 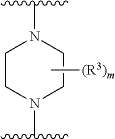
is 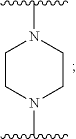 [SMILES: *N1CCN(*)CC1] 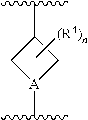
is 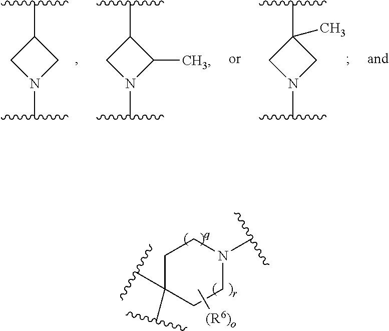
is

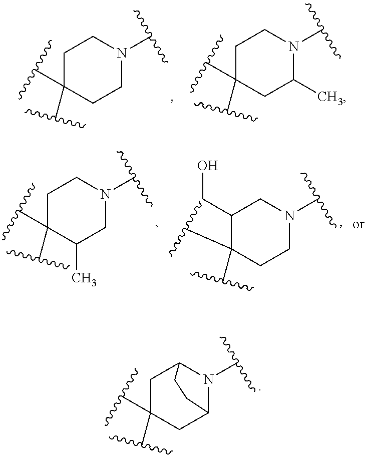

71. The compound of claim 70, wherein 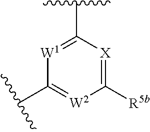
is

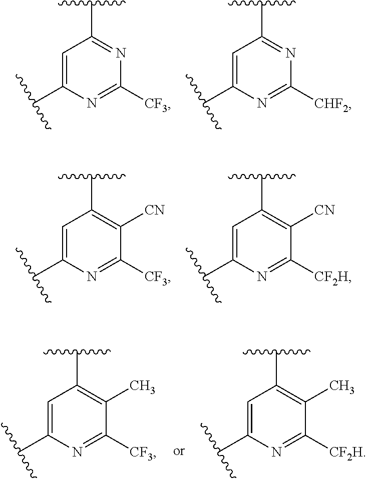

72. The compound or salt of claim 70 or 71, wherein 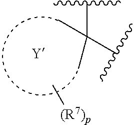
is

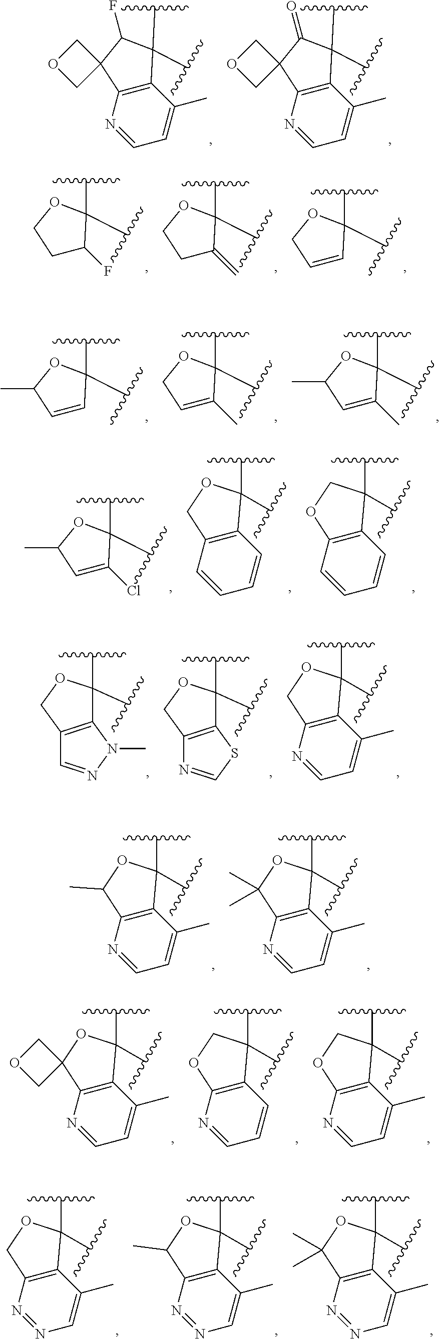

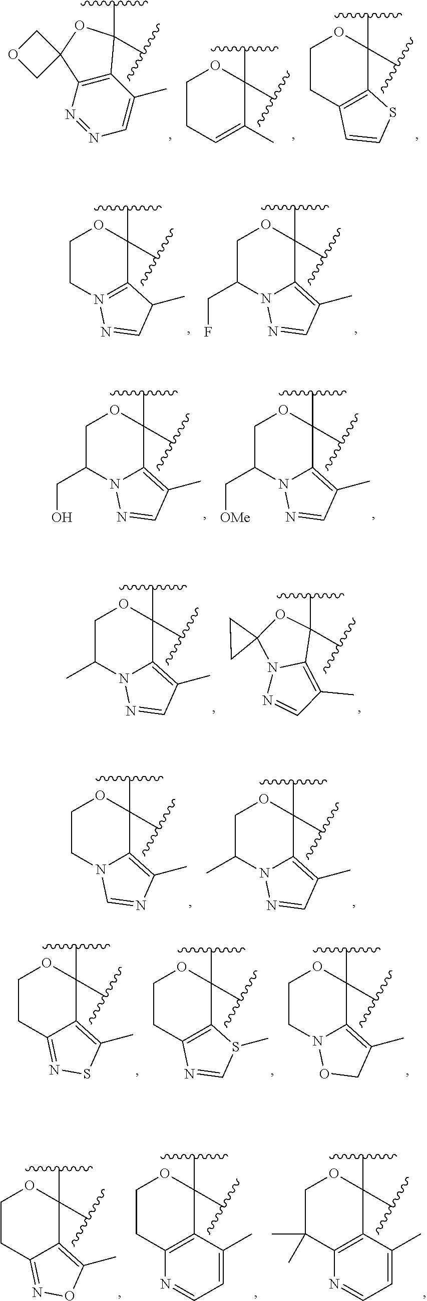

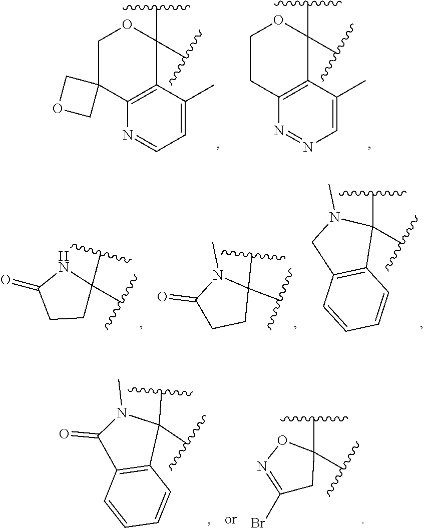

73. The compound or salt of any one of claims 1-30, wherein q is 1; r is 1; W¹ is CH, C-halo, C—CN, C—C₁₋₃alkyl, C—C₁₋₃haloalkyl, C—C₀₋₃alkyleneOH, or C—C₀₋₃alkylene-C₁₋₄alkoxy; W² is N; and X is N or C—R^{5a}.

74. The compound of claim 1, wherein the compound is a compound listed in Table A, or a pharmaceutically acceptable salt thereof.

75. The compound of claim 1, wherein the compound is a compound listed in Table C, or a pharmaceutically acceptable salt thereof.

76. A pharmaceutical composition comprising the compound or salt of any one of claims 1-75 and a pharmaceutically acceptable excipient.

77. A method of treating cancer in a subject in need of treatment, the method comprising administering to the subject a therapeutically effective amount of the compound or salt of any one of claims 1-76, or the composition of claim 77.

78. The method of claim 77, wherein one or more cancer cells express KRAS G12C mutant protein.

79. The method of claim 77 or 78, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

80. The method of claim 79, wherein the cancer is non-small cell lung cancer, colorectal cancer, pancreatic cancer, or solid tumor.

81. The method according to any one of claims 77-80, wherein the subject has a cancer that was determined to have one or more cells expressing the KRAS G12C mutant protein prior to administration of the compound, salt, or pharmaceutical composition.

82. The method according to any one of claims 77-81, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

83. The compound or salt of any one of claims 1-75, or the composition of claim 76 for use as a medicament.

84. The compound or salt of any one of claims 1-75, or the composition of claim 76 for use in treating cancer.

85. The compound or salt of any one of claims 1-75 or the pharmaceutical composition of claim 76 for use in treating cancer, wherein one or more cancer cells express KRAS G12C mutant protein.

86. The compound or salt of claim 84 or 85, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

87. The compound or salt of claim 86, wherein the cancer is non-small cell lung cancer, colorectal cancer, pancreatic cancer, or solid tumor.

88. Use of a compound or salt of any one of claims 1-75 or the pharmaceutical composition of claim 76 for the manufacture of a medicament for the treatment of cancer.

89. Use of a compound or salt of any one of claims 1-75 or the pharmaceutical composition of claim 76 in the preparation of a medicament for treating cancer, wherein one or more cancer cells express KRASG12C mutant protein.

90. The use of claim 88 or 89, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

91. The use of claim 90, wherein the cancer is non-small cell lung cancer, colorectal cancer, pancreatic cancer, or solid tumor.

92. A compound of Formula (B): 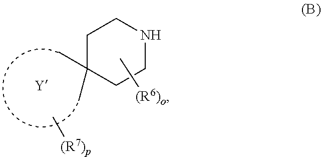
or a nitrogen-protected analog, or a pharmaceutically acceptable salt of any of the foregoing, wherein:
o is 0, 1, 2, 3, or 4;
p is 0, 1, 2, 3, 4, or 5;
each R⁶ s halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, C₁₋₄alkylene-N(R^{N1})₂, oxo, ═CH₂, C₃₋₇cycloalkyl, C₄₋₇cycloalkenyl, heterocycloalkyl having 4-7 total ring atoms and 1 or 2 heteroatoms independently selected from N, O and S, heterocycloalkenyl having 4-7 total ring atoms and 1 or 2 heteroatoms independently selected from N, O and S, spiro-C₃₋₇cycloalkyl, spiro-C₄₋₇cycloalkenyl, spiro-heterocycloalkyl having 4-7 total ring atoms and 1 or 2 heteroatoms independently selected from N, O and S, or spiro-heterocycloalkenyl having 4-7 total ring atoms and 1 or 2 heteroatoms independently selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms independently selected from N, O, and S, or fused-heterocycloalkenyl having 4-7 total ring atoms and 1 or 2 heteroatoms independently selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; wherein the cycloalkyl, cycloalkenyl, heterocycloalkyl or heterocycloalkenyl of each of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
each R⁷ independently is halo, C₀₋₂alkylene-CN, C₁₋₃alkyl, C₁₋₃haloalkyl, C₂₋₃alkenyl, C₀₋₃alkyleneOH, C₀₋₃alkylene-C₁₋₃alkoxy, C(═O)OC₁₋₃alkyl, oxo, ═CH₂, C₀₋₄alkylene-C₃₋₇cycloalkyl, C₀₋₄alkylene-heterocycloalkyl having 3-7 total ring atoms and 1-3 heteroatoms independently selected from N, O, and S; C₀₋₄alkylene-C₆₋₁₀aryl, C₀₋₄alkylene-heteroaryl having 4-7 total ring atoms and 1-3 heteroatoms independently selected from N, O, and S; spiro-C₃₋₇cycloalkyl, spiro-heterocycloalkyl having 4-7 total ring atoms and 1 or 2 heteroatoms independently selected from N, O and S; or two vicinal R⁷ together with the atoms to which they are attached form fused-C₃₋₇cycloalkyl, fused-heterocycloalkyl having 3-7 total ring atoms and 1-3 heteroatoms independently selected from N, O and S, fused-C₆₋₁₀aryl, or fused-heteroaryl having 5-7 total ring atoms and 1-3 heteroatoms independently selected from N, O, and S; wherein the cycloalkyl, heterocycloalkyl, aryl, or heteroaryl of each of the foregoing is unsubstituted or substituted with 1-3 substituents, and each substituent independently is halo, CN, oxo, or C₁₋₃alkyl; and
each R^{N1} independently is H or C₁₋₄alkyl.

93. A method of preparing the compound or salt of any one of claims 1-75, comprising converting a compound of claim 92, a protected analog of any of the foregoing, or a pharmaceutically acceptable salt of any of the foregoing, into a compound of any one of claims 1-75.
